(19) United States
(12) Patent Application Publication
(10) Pub. No.: US 2021/0169896 A1
(43) Pub. Date: Jun. 10, 2021

(54) A CROSS-LINKED PYRROLOBENZODIAZEPINE DIMER (PBD) DERIVATIVE AND ITS CONJUGATES

(71) Applicant: Hangzhou DAC Biotech Co., Ltd., Hangzhou, Zhejiang (CN)

(72) Inventors: Robert Yongxin ZHAO, Lexington, MA (US); Xiaotao ZHUO, Hangzhou, Zhejiang (CN); Qingliang YANG, Hangzhou, Zhejiang (CN); Linyao ZHAO, Hangzhou, Zhejiang (CN); Yuanyuan HUANG, Hangzhou, Zhejiang (CN); Hangbo YE, Hangzhou, Zhejiang (CN); Chengyu YANG, Hangzhou, Zhejiang (CN); Jun LEI, Hangzhou, Zhejiang (CN); Shun GAI, Hangzhou, Zhejiang (CN); Huihui GUO, Hangzhou, Zhejiang (CN); Junxiang JIA, Hangzhou, Zhejiang (CN); Lu BAI, Hangzhou, Zhejiang (CN); Hongsheng XIE, Hangzhou, Zhejiang (CN); Xiaomai ZHOU, Hangzhou, Zhejiang (CN); Zhixiang GUO, Hangzhou, Zhejiang (CN); Wenjun LI, Hangzhou, Zhejiang (CN); Mingjun CAO, Hangzhou, Zhejiang (CN); Jun ZHENG, Hangzhou, Zhejiang (CN); Zhicang YE, Hangzhou, Zhejiang (CN); Yanlei YANG, Hangzhou, Zhejiang (CN)

(73) Assignee: Hangzhou DAC Biotech Co., Ltd., Hangzhou, Zhejiang (CN)

(21) Appl. No.: 17/256,034

(22) PCT Filed: Jul. 5, 2018
(86) PCT No.: PCT/CN2018/094586

§ 371 Date: Dec. 24, 2020

(51) Int. Cl. A61K 31/5517; C07D 519/00; A61K 45/06; A61K 47/55; A61K 47/68; A61K 47/54; A61P 35/00

(52) U.S. Cl. CPC A61K 31/5517; C07D 519/00; A61K 45/06; A61P 35/00; A61K 47/6803; A61K 47/545; A61K 47/55

(57) ABSTRACT

The present invention relates to novel cross-linked cytotoxic agents, pyrrolobenzodiazepine dimer (PBD) derivatives, and their conjugates to a cell-binding molecule, a method for preparation of the conjugates and the therapeutic use of the conjugates.

### FIELD OF THE INVENTION

The present invention relates to novel cross-linked cytotoxic agents, pyrrolobenzodiazepine dimer (PBD) derivatives, and their conjugates to a cell-binding molecule, a method for preparation of the conjugates and the therapeutic use of the conjugates.

### BACKGROUND OF THE INVENTION

Antibody-drug conjugates (ADCs) have great promise as anti-cancer drugs, with four ADCs have been approved by FDA and over 100 others in clinical investigation by using several different classes of cytotoxic payloads such as maytansines (Zhao, Robert Y, et al 2011 J. Med. Chem. 54, 3606; Widdison, W.; et al 2006 J. Med. Chem., 49, 4392-4408); auristatins and its parent compound dolastatin (Doronina, S. O. et al, Nat. Biotechnol. 2003, 21 (7), 778-784; Maderna, A., 2014 J. Med. Chem., 57(24), 10527-10543.); calicheamycins (Ricart, A. D. 2011 Clin Cancer Res 2011; 17:6417-6427; Ricart A. D, et al Nat Clin Pract Oncol. 2007; 4:245-255), duocarmycins and its analog CC-1065 (Elgersma, R. C. 2015 Mol. Pharmaceutics, 12 (6), 1813-1835; Zhao, Robert Y. et al, 2012 J. Med. Chem 55, 766-782), tubulysins (Li, J. Y. et al, 2016 Cancer Cell. 29(1):117-29; Tumey, L. N. et al 2016 ACS Med Chem Lett. 7(11):977-982; Huang Y. Y., Zhao, Robert Y. et al, Med Chem. #44, 249^{th} ACS National Meeting, Denver, Colo., Mar. 22˜26, 2015; WO2014009774), Eribulins (US20170252458); camptothecin and its analog SN-38 (Cardillo. T. M, et al, 2015 Bioconjug Chem. 26(5):919-31; Cardillo. T. M, et al, 2011 Clin Cancer Res. 17(10): 3157-69; Doi T, et al, 2017 Lancet Oncol. 18(11):1512-1522; PCT/JP2013/006069; US20160333112); amanitin (Moldenhauer G, et al, J. Natl. Cancer Inst. 2012; 104(8):622-34; Zhao, Robert Y. et al, PCT/IB2016/052246); vinblastine (Laguzza, B. C. et al, 1989 J. Med. Chem., 32, 548-555; Starling, J. J., et al 1992 Bioconj. Chem., 3, 315-322); doxorubicin (Yang, H. M. and Reisfeld, R. A., 1988 Proc. Natl. Acad. Sci. USA, 85, 1189-93; Sapra, P., et al 2005 Clin Cancer Res 11, 5257-64; Geretti, E. et al, 2015 Mol Cancer Ther 14, 2060-71); Cryptophycin (Verma, V. A. et al, 2015 Bioorg. Med. Chem. Lett., 25 (4), 864-8); Eribulin (Furuuchi, K. et al, poster MORAb-202, 8^{th} World ADC, Sep. 19, 2017; US2017252458); and pyrrolobenzodiazepine dimers (PBDs) (Mantaj, J. et al 2016 Angew. Chem. Int. Ed. 55, 2-29; D. Antonow and D. Thurston, Chem. Rev. 2011, 111, 2815-2864). In recent years, there has been increasing applications of the PBD class of cytotoxic payloads as ADC therapeutics (Kung Sutherland, M. S., et al 2013 Blood 122, 1455-63; Jeffrey, S. C., 2013, Bioconjug Chem. 1256-63); Saunders, L. R. et al 2015 Sci Transl Med., 7(302):302ra136; Flynn, M. J. et al 2016 Mol Cancer Ther., 15(11), 2709-272; Zhao, Robert Y., WO2015028850; Donnell, A. F. et al 2017 Bioorg Med Chem Lett 27, 5267-5271) due to PBDs' extreme potency against most of the US National Cancer Institute's (NCI's) NCI 60-tumor-cell line panels along with unique mechanism of antitumor antibiotics activity (Mantaj, J. et al 2016 Angew. Chem. Int. Ed. 55, 2-29).

As shown in structures below, all PBDs are usually conjugated to an antibody with one conditionally stable linker, because it is normally believed that linkers of ADCs must be stable while the ADC is circulating in the blood to limit off-target toxicity, but allow for release of the drug once it is inside the target cancer cells (Alain Beck 2017 Nature Reviews Drug Discovery, doi:10.1038/nrd.2016.268). Unfortunately the most advanced in clinic evaluation of Vadastuxi-mab talirine (CD33 antibody-PBD conjugate having a conditionally stable linker) (Stein, E. M., et al, 2018 Blood 131, 387-96) experienced a set-back because of liver toxicity that apparently killed four patients in the early stage trials (www.xconomy.com/seattle/2017/06/19).

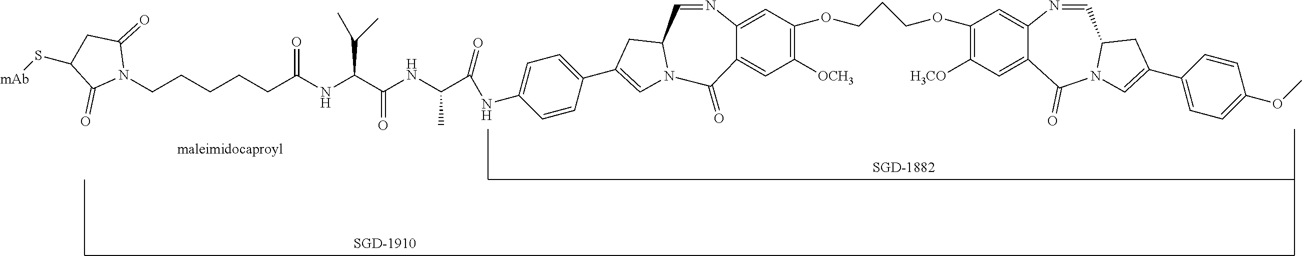

The SGD-1882 conjugate, studied by Seattle Genetics,

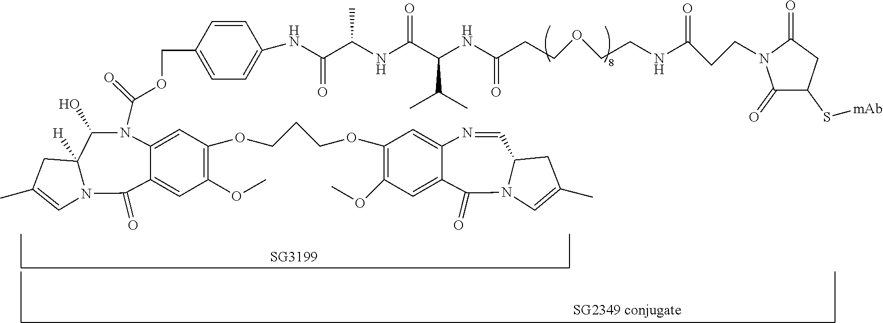

The SG3249 conjugate studied by ADC Therapeutics Ltd and by MedImmune/AstraZeneca.

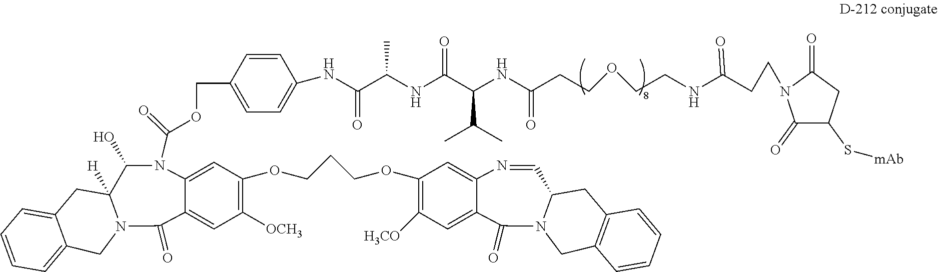

The D-212 conjugate studied by Cellerant Therapeutics,

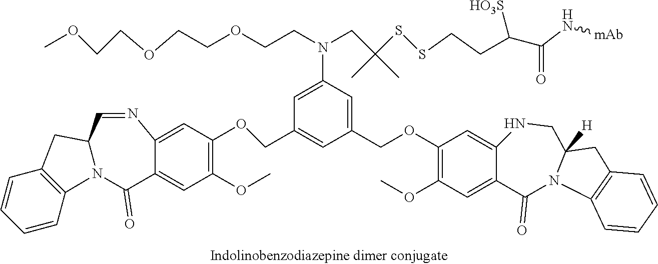

An IGN conjugate, studied by ImmunoGen, Inc.,

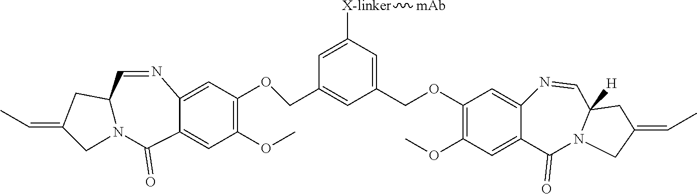

A Tomaymysin dimer (a PBD) conjugates for Sanofi,

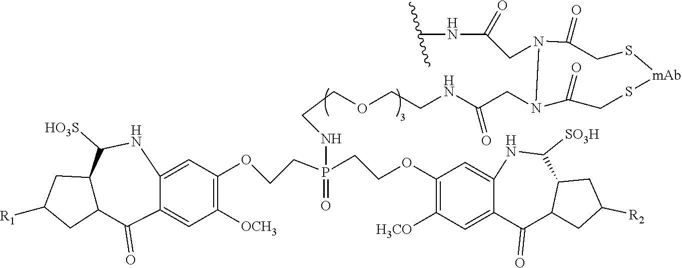

A PBD dimer conjugate, studied by Hangzhou DAC Biotech Co., Ltd,

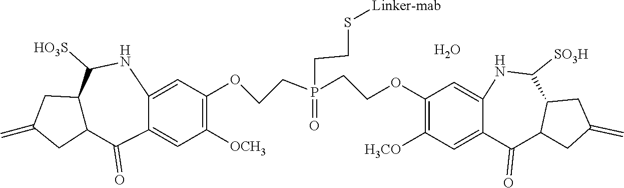

A PBD dimer conjugate, studied by Hangzhou DAC Biotech Co., Ltd,

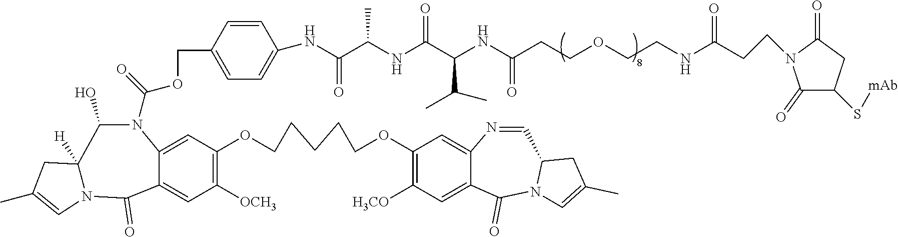

The SG-3199 conjugate, studied by Stemcentrx/Abbvie and by ADC Therapeutics Ltd.

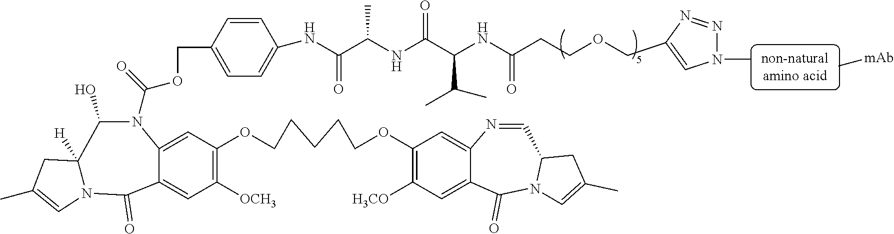

The SG-3199 conjugate, studied by Allozyne, Inc.

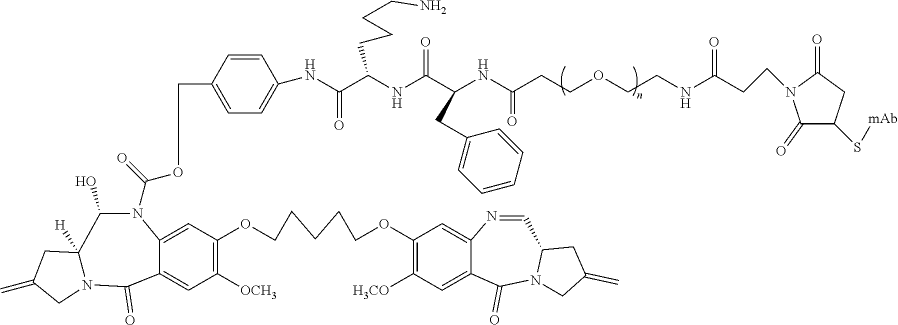

The SG2057 conjugate, used by Genentech Inc.

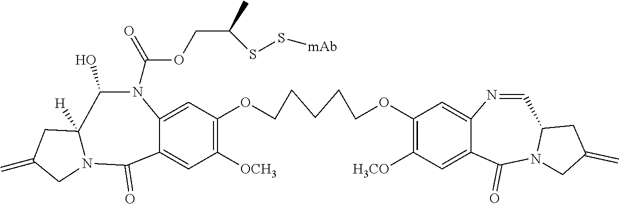

A PBD conjugate via a traceless linker by Genentech Inc

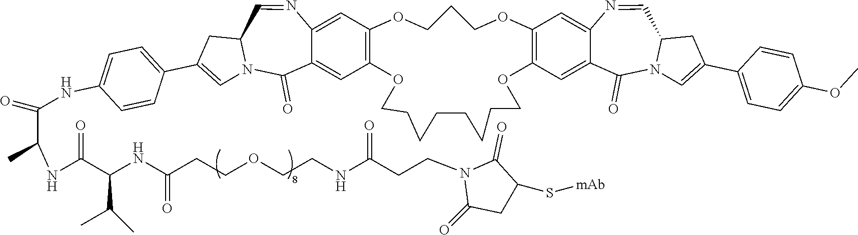

A PBD dimer conjugate, studied by Bristol-Myers Squibb.

Here we are disclosing the conjugation of PBD dimer derivatives through a dual cross linker that attached both N10 positions of the PBD dimer, thus DNA alkylation sites of the imine warheads are in the forms of prodrugs wherein the conjugate linkers have to be decomposed prior to converting back the potency. Indeed, the application of the dual cross-linking prodrug strategy of PBD conjugation demonstrated much wider therapeutic windows than the single linkage of PBD conjugates both in vitro and in vivo. It therefore may have much potentially better antitumor antibiotic activities for PBD conjugates in clinic applications.

### SUMMARY OF THE INVENTION

The first embodiment of this invention is to disclose a dual linkage of conjugation of PBDs via attachment of both N10 positions of pyrrolo[2,1-c][1,4]benzodiazepine derivatives to a cell binding molecule, as shown in Formula (I), for targeted treatment of cell proliferation.

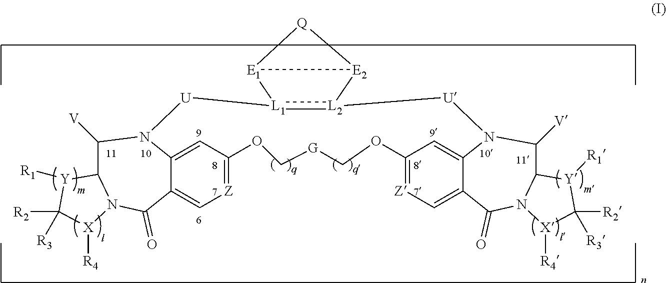

or their pharmaceutically acceptable salts, hydrates, or hydrated salts, or the polymorphic crystalline structures of these compounds or their optical isomers, racemates, diastereomers or enantiomers;

wherein:

----- represents an optional single bond or can be absent;

[symbol] represents an optional single bond or a double bond;

V and V′, the same or different, are independently selected from the group consisting of H, OH, —NHOH; OR₅ (an ether); OCOR₅ (an ester); OCOOR₅ (a carbonate); NR₅R₅′, NR₅COR₅′, or NR₅NR₅′NR₅″ (an amine); OCONR₅R₅′ (a carbamate); NR₅(C═NH)NR₅′R₅″ (a guanidinum); NR₅CONR₅′R₅″ (a urea); OCSNHR₅ (a thiocarbamate); —SH (a thiol); —SR₅ (a sulfide); SOR₅ a sulphoxide (a sulphoxide); SOOR₅ (a sulfone); SO₃, HSO₃, HSO₂, or a salt of HSO³⁻, SO₃²⁻ or —HSO₂⁻ (a sulphite); OSO₃ (a bisulphite); NR₅SOOR₅′ (a sulfonamide); H₂S₂O₅ or a salt of S₂O₅²⁻ (a metabisulfite); PO₃SH₃, PO₂S₂H₂, POS₃H₂, PS₄H₂ or a salt of PO₃S³⁻, PO₂S₂³⁻, POS₃³⁻, PS₄³⁻ (mono-, di-, tri-, and tetra-thiophosphate); (R₅O)₂POSR₅′ (a thiophosphate ester); HS₂O₃ or a salt of S₂O₃²⁻ (a thiosulfate); HS₂O₄ or a salt of S₂O₄²⁻ (a dithionite); P(═S)(OR₅)(S)(OH) (a phosphorodithioate) or a salt thereof form with a cation; —NR₅OR₅′ (a hydroxylamine derivative); R₅C(═O)NOH (a hydroxamic acid) or a salt formed with a cation; HOCH₂SO₂⁻, or its salts (a formaldehyde sulfoxylate); NR₅COR_{5′} (an amide); N₃ (an azido); CN (a cyano); X (a halo); C(R₅)(R_{5′})(R_{5″}) (a trialkyl), OP(O)(OR₅)(NHR_{5′}) or OP(O)(NHR₅)(NHR_{5′}) (a phosphoramidate (phosphoramidic acid), or P(R₅)(R_{5′})(R_{5″}) triarylphosphonium; Aa (an amino acid), or NR₅CO(Aa)ₜ (a peptide), wherein Aa is an amino acid or a polypeptide containing between t=1˜100 amino acid units; an aminoacid-derived group, such as an α-, β-, γ-, or ω-aminoacid, or an unnatural aminoacid; where R₅, R₅′ and R₅″ are described below;

l, m, q, l′, m′ and q′ are independently a number of 0, 1, 2, 3, 4, or 5; n is 1˜30;

X, X′, Y and Y′ the same or different, independently, represent N, O, S, an alkyl, such as CH₂ or CHR₅, an alkene, such as ═CH— or ═CR₅—, an ether, such as —C(OR₅)H—;

Z and Z′ the same or different, independently, represent N, CH, CR₅, COH, CNH₂, CNHR₅, or COR₅, or Z and Z′ link together with —COR₅OC—. R₅ is independently selected from C₁˜C₈ alkyl and aryl;

R₁, R₂, R₃, R₄, R₁′, R₂′, R₃′, and R₄′ are the same or different and independently chosen from —H, an optionally substituted linear, branched or cyclic alkyl, alkenyl or alkynyl having from 1 to 10 carbon atoms, —(OCH₂CH₂)ₜR₅ (a polyethylene glycol unit), halogen, NH(C═NH)NH₂ (a guanidinium), —OR₅, —NR₅R₅′, —NO₂, —NCO, —NR₅COR₅′, —SR₅, —SOR₅ (a sulfoxide), —SO₂R (a sulfone), —SO₃⁻M⁺ or —SO₃H (a sulfonate), —OSO₃⁻M⁺ or OSO₃H (a sulfate), —SO₂NR₅R₅′ (a sulfonamide), CN (a cyano), N₃ (an azido), —COR₅, —OCOR₅, —OCONR₅R₅′, CF₃, OR₅, Aryl, heterocycle, or P(O)R₅R₅′R₅″ and the linking group (L″) with the reactive group or a cell binding agent bonded thereto when Q, Q′ and T are absent;

R₅, R₅′ and R₅″ are independently selected from H, C₁˜C₈ of alkyl, alkenyl, alkinyl, heteroalkyl, aryl, arylalkyl, carbonyl, or pharmaceutical salts;

In addition, R₁ and R₂ join together, or R₁′ and R₂′ join together form a ═O (ketone), ═S, ═NR, —C(═O)R, or a double bond containing group ═CR₅R₅′; and R₁ and R₂ join together, or R₁′ and R₂′ join together, or R₃ and R₄ join together, or R₃′ and R₄′ join together form a C₃-C₁₂ aromatic, heterocyclic, or heteroaryl ring;

G is —CH₂—, O, —N(R₅)—, S, —P(O)(OR₅)—, —P(O)(NR₅R_{5′})—,

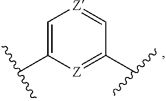

wherein Z and Z′ are defined above;

U and U′ are independently C(O), C(O)O, C(O)NH, C(O)N(R₅), C(═NH), C(═NH)O, C(═NH)NH, C(═NH)N(R₅), —C═N—, C(═S), C(O)S, C(S)NH, C(S)N(R₅), S(O), S(O)O, S(O)NH, S(O)(OR₅), S(O)(N(R₅)), S(O₂), S(O₂)O, P(O)(OR₅), P(O)(OR₅)O, P(O)(NH₂), P(O)(NR₅R_{5′}), P(O)(OR₅)NH—, P(O)(OR₅)NR_{5′}—, P(O)(N(R₅R_{5′})(N(R₅), P(S)(OR₅), P(S)(OR₅)O, P(S)(NH₂), P(S)(NR₅R_{5′}), P(S)(OR₅)NH—, P(S)(OR₅)NR_{5′}—, P(S)(N(R₅R_{5′})N(R₅), R₅, R₅O;

E₁ and E₂ are independently S, R₅S, C(O)S, C(O)NH, C(O)O, C(O)R₅S, C(═NH)NH, C(═NH)N(R₅), C(═NH)S, —C═N—, C(═S)S, C(O)S, C(═S)NH, C(═S)N(R₅), Ar—S, NC(O)CH₂S, ArC(O)CH₂S, S—S,

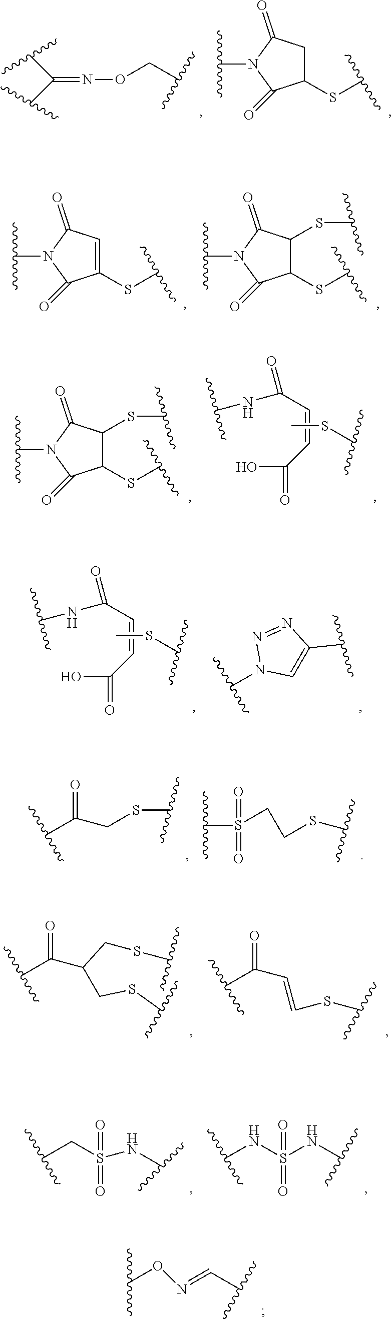

wherein the chemical bond in the middle of two atoms means it can link either adjoining two atoms;

L₁, L₂ and L′ are independently a linker, or a linker which has a functional group on the linker that enables reaction with a cell-binding agent (CBA), Q. L₁, L₂ and L′ are independently preferred a releasable linker, which has the formula of -Ww-(Aa)r-Tt-; or -Ww-(Aa)r-Tt-Q; or Q-Ww-(Aa)r-Tt-; wherein: —W— is a Stretcher unit; w is O or 1; -Aa- is independently an Amino Acid unit; r is independently an integer ranging from 0 to 100; -T- is a Spacer unit, which can be a linear alkyl or branched alkyl, or polyethylene glycol spacer; and t is 0, or 1˜100. The Stretcher unit W may independently contain a self-immolative spacer, peptidyl units, a hydrazone bond, a disulfide, an ester, or a thioether bond; w is 1 or 2 or 3; Preferably Li and L₂ are independently selected from O, NH, N, S, P, NNH, NHNH, N(R₃), N(R₃)N(R_{3′}), CH, CO, C(O)NH, C(O)O, NHC(O)NH, NHC(O)O, polyethyleneoxy unit of formula (OCH₂CH₂)ₚOR₃, or (OCH₂CH—(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚ—COOR₃, wherein p and p′ are independently an integer selected from 0 to about 1000, or combination thereof; C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or (Aa)ᵣ, r=1-12 (one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;

Q is a cell binding molecule, or a functional group that enables reaction with a cell-binding agent, or a functional group capable of reacting with a linker attached on a cell binding agent. The function group is chosen from a thiol, an amine, a hydrazine, an alkoxylamino, a disulfide substituent, a maleimido, a haloacetyl group, an N-hydroxy succinimide ester, a keton, an ester, an aldehyde, an alkynyl, an alkenyl, or protected thiol or disulfide group, such as SAc, SSR₁ or SSAr. Ar is aromatic group or hetero aromatic group. Q is preferably a cell-binding agent/molecule, selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and a binding peptide, protein, or small molecule attached on albumin, a polymer, a dendrimer, a liposome, a nanoparticle, a vesicle, or a (viral) capsid.

In a second embodiment, the present invention discloses mono-linkage of conjugation of PBDs derivatives to a cell binding molecule, as shown in Formula (II), (III) and (IV), for targeted treatment of cell proliferation.

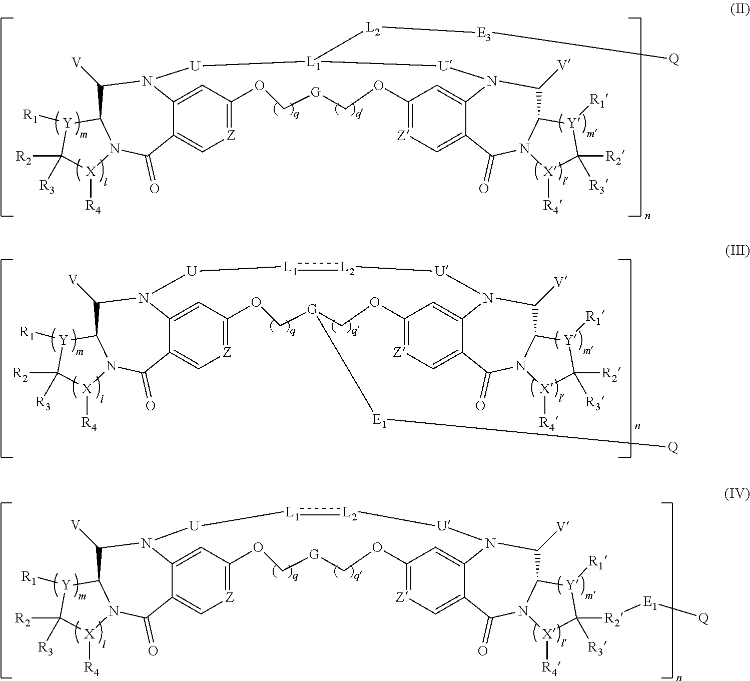

wherein [symbol] , -----, X, X′, Y, Y′, Z, Z′, l, l′, m, m′, n, q, q′, R₁, R₁′, R₂, R₂′, R₃, R₃′, R₄, R₄′, V, V′, U, U′ L₁, L₂, G, Q, E₁, and E₂ are the same defined as in Formula (I).

In a third embodiment, the present invention discloses a therapeutic composition comprising: (1) an effective amount of one or more of the pyrrolo[2,1-c][1,4]benzodiazepine derivatives linked to a cell binding agent as the conjugate structure of Formula (I), (II), (III) or (IV); and (2) a pharmaceutically acceptable carrier, diluent, or excipient, of Formula (I)˜(IV) of the application, to kill target cells or tissues containing target cells.

### BRIEF DESCRIPTION OF THE DRAWINGS

FIG. 1 shows the synthesis of cross-linked benzodiazepine dimers.

FIG. 2 shows the synthesis of an antibody conjugate of cross-linked benzodiazepine dimers.

FIG. 3 shows the synthesis of cross-linked benzodiazepine dimers.

FIG. 4 shows the synthesis of cross-linked benzodiazepine dimers and a conjugate.

FIG. 5 shows the synthesis of an antibody conjugate of cross-linked benzodiazepine dimers.

FIG. 6 shows the synthesis of an antibody conjugate of cross-linked benzodiazepine dimers.

FIG. 7 shows the synthesis of benzodiazepine dimers.

FIG. 8 shows the synthesis of cross-linked benzodiazepine dimers and a conjugate.

FIG. 9 shows the synthesis of benzodiazepine dimers.

FIG. 10 shows the synthesis of an antibody conjugate of cross-linked benzodiazepine dimers.

FIG. 11 shows the synthesis of benzodiazepine dimers.

FIG. 12 shows the synthesis of conjugates of benzodiazepine dimers and their linkers.

FIG. 13 shows the synthesis of the cross-linkers for conjugates of benzodiazepine dimers.

FIG. 14 shows the synthesis of the cross-linkers for conjugates of benzodiazepine dimers.

FIG. 15 shows the synthesis of the cross-linkers for conjugates of benzodiazepine dimers.

FIG. 16 shows the synthesis of conjugates of cross-linked benzodiazepine dimers.

FIG. 17 shows the synthesis of the intermediates for conjugates of benzodiazepine dimers.

FIG. 18 shows the synthesis of conjugates of cross-linked benzodiazepine dimers.

FIG. 19 shows the synthesis of conjugates of cross-linked benzodiazepine dimers.

FIG. 20 shows the synthesis of conjugates of cross-linked benzodiazepine dimers.

FIG. 21 shows the synthesis of conjugates of cross-linked benzodiazepine dimers.

FIG. 22 shows the synthesis of the intermediates for conjugates of benzodiazepine dimers.

FIG. 23 shows the synthesis of cross-linked benzodiazepine dimers.

FIG. 24 shows the synthesis of conjugates of cross-linked benzodiazepine dimers.

FIG. 25 shows the synthesis of cross-linked benzodiazepine dimers.

FIG. 26 shows the synthesis of cross-linked benzodiazepine dimers.

FIG. 27 shows the synthesis of cross-linked benzodiazepine dimers.

FIG. 28 shows the synthesis of the intermediates for conjugates of benzodiazepine dimers.

FIG. 29 shows the synthesis of conjugates of cross-linked benzodiazepine dimers.

FIG. 30 shows the synthesis of the intermediates for conjugates of benzodiazepine dimers.

FIG. 31 shows the synthesis of the intermediates for conjugates of benzodiazepine dimers.

FIG. 32 shows the synthesis of conjugates of benzodiazepine dimers.

FIG. 33 shows the in vivo activity of conjugates of cross-linked benzodiazepine dimers.

FIG. 34 shows the hematoxylin and eosin stainings of mouse liver tissues after dosing the conjugates CC-4, CC-29, T-DM1 and PBS buffer in ICR mice on day 5.

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having C₁-C₈ (1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methyl-hexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N₃, —NH₂, —NH(R′), —N(R′)₂ and —CN; where each R′ is independently selected from —C₁-C₈ alkyl and aryl.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4,5], [5,5], [5,6] or [6,6] system, or 9 or 10 ring atoms arranged as a bicycle [5,6] or [6,6] system. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′, —S(O)₂R′, —OH, -halogen, —N₃, —NH₂, —NH(R′), —N(R′)₂ and —CN; where each R′ is independently selected from —C₁-C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexylynyl, heptynyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or Ar refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″,—NO₂, —S(O)R′, —S(O)₂R′, —S(O)₂OR′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1,2,4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” includes, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate.

“Leaving group” refers to a functional group that can be substituted by another functional group. Such leaving groups are well known in the art, and examples include, a halide (e.g., chloride, bromide, and iodide), methanesulfonyl (mesyl), p-toluenesulfonyl (tosyl), trifluoromethylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CDI, 1,1′-carbonyldiimidazole; DCC, dicyclohexylcarbodiimide; DCE, dichloroethane; DCM, dichloromethane; DEAD is diethylazodicarboxylate, DIAD, diisopropylazodicarboxylate; DIBAL-H, diisobutyl-aluminium hydride; DIPEA or DEA, diisopropylethylamine; DEPC, diethyl phosphorocyanidate; DMA, N,N-dimethyl acetamide; DMAP, 4-(N, N-dimethylamino)pyridine; DMF, N,N-dimethylformamide; DMSO, dimethylsulfoxide; DTPA is diethylenetriaminepentaacetic acid; DTT, dithiothreitol; EDC, 1-(3-dimethylaminopropyl)-3-ethylcarbodiimide hydrochloride; ESI-MS, electrospray mass spectrometry; EtOAc is ethyl acetate; Fmoc is N-(9-fluorenylmethoxycarbonyl); HATU, O-(7-azabenzotriazol-1-yl)-N, N, N′, N′-tetramethyluronium hexafluorophosphate; HOBt, 1-hydroxybenzotriazole; HPLC, high pressure liquid chromatography; NHS, N-Hydroxysuc-cinimide; MeCN is acetonitrile; MeOH is methanol; MMP, 4-methylmorpholine; PAB, p-aminobenzyl; PBS, phosphate-buffered saline (pH 7.0-7.5); Ph is phenyl; phe is L-phenylalanine; PyBrop is bromo-tris-pyrrolidino-phosphonium hexafluorophosphate; PEG, polyethylene glycol; SEC, size-exclusion chromatography; TCEP, tris(2-carboxyethyl)phosphine; TFA, trifluoroacetic acid; THF, tetrahydrofuran; Val, valine; TLC is thin layer chromatography; UV is ultraviolet.

The “amino acid(s)” can be natural and/or unnatural amino acids, preferably alpha-amino acids. Natural amino acids are those encoded by the genetic code, which are alanine, arginine, asparagine, aspartic acid, cysteine, glutamic acid, glutamine, glycine, histidine, isoleucine, leucine, lysine, methionine, phenylalanine, proline, serine, threonine, tyrosine. tryptophan and valine. The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3,4-dihydroxyphenylalanine (DOPA), and O-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form. When 1-8 amino acids are used in this patent application, amino acid sequence is then preferably a cleavage recognition sequence for a protease. Many cleavage recognition sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference. In particular, the sequence is selected from the group consisting of Val-Cit, Ala-Val, Ala-Ala, Val-Val, Val-Ala-Val, Lys-Lys, Ala-Asn-Val, Val-Leu-Lys, Cit-Cit, Val-Lys, Ala-Ala-Asn, Asp-Lys, Asp-Glu, Glu-Lys, Lys, Cit, Ser, and Glu.

The “glycoside” is a molecule in which a sugar group is bonded through its anomeric carbon to another group via a glycosidic bond. Glycosides can be linked by an O- (an O-glycoside), N- (a glycosylamine), S- (a thioglycoside), or C- (a C-glycoside) glycosidic bond. Its core the empirical formula is Cₘ(H₂O)ₙ (where m could be different from n, and m and n are <36), Glycoside herein includes glucose (dextrose), fructose (levulose) allose, altrose, mannose, gulose, iodose, galactose, talose, galactosamine, glucosamine, sialic acid, N-acetylglucosamine, sulfoquinovose (6-deoxy-6-sulfo-D-glucopyranose), ribose, arabinose, xylose, lyxose, sorbitol, mannitol, sucrose, lactose, maltose, trehalose, maltodextrins, raffinose, Glucuronic acid (glucuronide), and stachyose. It can be in D form or L form, 5 atoms cyclic furanose forms, 6 atoms cyclic pyranose forms, or acyclic form, α-isomer (the —OH of the anomeric carbon below the plane of the carbon atoms of Haworth projection), or a β-isomer (the —OH of the anomeric carbon above the plane of Haworth projection). It is used herein as a monosaccharide, disaccharide, polyols, or oligosaccharides containing 3-6 sugar units.

The term “antibody,” as used herein, refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g. IgG, IgE, IgM, IgD, IgA and IgY), class (e.g., IgG1, IgG2, IgG3, IgG4, IgA1 and IgA2) or subclass of immunoglobulin molecule. The immunoglobulins can be derived from any species. Preferably, however, the immunoglobulin is of human, murine, or rabbit origin. Antibodies useful in the invention are preferably monoclonal, and include, but are not limited to, polyclonal, monoclonal, bispecific, human, humanized or chimeric antibodies, single chain antibodies, Fv, Fab fragments, F(ab′) fragments, F(ab′)₂ fragments, fragments produced by a Fab expression library, anti-idiotypic (anti-Id) antibodies, CDR's, and epitope-binding fragments of any of the above which immunospecifically bind to cancer cell antigens, viral antigens or microbial antigens.

An “enantiomer”, also known as an “optical isomer”, is one of two stereoisomers that are mirror images of each other that are non-superposable (not identical), much as one's left and right hands are the same except for being reversed along one axis (the hands cannot be made to appear identical simply by reorientation). A single chiral atom or similar structural feature in a compound causes that compound to have two possible structures which are non-superposable, each a mirror image of the other. The presence of multiple chiral features in a given compound increases the number of geometric forms possible, though there may be some perfect-mirror-image pairs. Enantiopure compounds refer to samples having, within the limits of detection, molecules of only one chirality. When present in a symmetric environment, enantiomers have identical chemical and physical properties except for their ability to rotate plane-polarized light (+/−) by equal amounts but in opposite directions (although the polarized light can be considered an asymmetric medium). They are sometimes called optical isomers for this reason. A mixture of equal parts of an optically active isomer and its enantiomer is termed racemic and has zero net rotation of plane-polarized light because the positive rotation of each (+) form is exactly counteracted by the negative rotation of a (−) one. Enantiomer members often have different chemical reactions with other enantiomer substances. Since many biological molecules are enantiomers, there is sometimes a marked difference in the effects of two enantiomers on biological organisms. In drugs, for example, often only one of a drug's enantiomers is responsible for the desired physiologic effects, while the other enantiomer is less active, inactive, or sometimes even productive of adverse effects. Owing to this discovery, drugs composed of only one enantiomer (“enantiopure”) can be developed to enhance the pharmacological efficacy and sometimes eliminate some side effects.

Isotopes are variants of a particular chemical element which differs in neutron number. All isotopes of a given element have the same number of protons in each atom. Each atomic number identifies a specific element, but not the isotope; an atom of a given element may have a wide range in its number of neutrons. The number of nucleons (both protons and neutrons) in the nucleus is the atom's mass number, and each isotope of a given element has a different mass number. For example, carbon-12, carbon-13 and carbon-14 are three isotopes of the element carbon with mass numbers 12, 13 and 14 respectively. The atomic number of carbon is 6, which means that every carbon atom has 6 protons, so that the neutron numbers of these isotopes are 6, 7 and 8 respectively. Hydrogen atom has three isotopes of protium (¹H), deuterium (²H), and tritium (³H), which deuterium has twice the mass of protium and tritium has three times the mass of protium. Isotopic substitution can be used to determine the mechanism of a chemical reaction and via the kinetic isotope effect. Isotopic substitution can be used to study how the body affects a specific xenobiotic/chemical after administration through the mechanisms of absorption and distribution, as well as the metabolic changes of the substance in the body (e.g. by metabolic enzymes such as cytochrome P450 or glucuronosyltransferase enzymes), and the effects and routes of excretion of the metabolites of the drug. This study is called pharmacokinetics (PK). Isotopic substitution can be used to study of the biochemical and physiologic effects of drugs. The effects can include those manifested within animals (including humans), microorganisms, or combinations of organisms (for example, infection). This study is called pharmacodynamics (PD). The effects can include those manifested within animals (including humans), microorganisms, or combinations of organisms (for example, infection). Both together influence dosing, benefit, and adverse effects of the drug. isotopes can contain a stable (non-radioactive) or an unstable element. Isotopic substitution of a drug may have a different therapeutical efficacy of the original drug.

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

“Therapeutically effective amount” means an amount of a compound/medicament according to the present invention effective in preventing or treating the herein referred pathological condition.

The term “patient”, or “patient in need thereof”, is intended for an animal or a human being affected or likely to be affected with the herein referred pathological condition. Preferably, the patient is human.

In the context of cancer, the term “treating” includes any or all of: preventing growth of tumor cells or cancer cells, preventing replication of tumor cells or cancer cells, lessening of overall tumor burden and ameliorating one or more symptoms associated with the disease.

In the context of an autoimmune disease, the term “treating” includes any or all of: preventing replication of cells associated with an autoimmune disease state including, but not limited to, cells capable of producing an autoimmune antibody, lessening the autoimmune-antibody burden and ameliorating one or more symptoms of an autoimmune disease.

In the context of an infectious disease, the term “treating” includes any or all of: preventing the growth, multiplication or replication of the pathogen that causes the infectious disease and ameliorating one or more symptoms of an infectious disease.

Examples of a “mammal” or “animal” include, but are not limited to, a human, rat, mouse, guinea pig, monkey, pig, goat, cow, horse, dog, cat, bird and fowl.

The term “compound”, “cytotoxic agent”, “cytotoxic compound,” “cytotoxic dimer” and “cytotoxic dimer compound” are used interchangeably. They are intended to include compounds for which a structure or formula or any derivative thereof has been disclosed in the present invention or a structure or formula or any derivative thereof that has been incorporated by reference. The term also includes, stereoisomers, geometric isomers, tautomers, solvates, metabolites, salts (e.g., pharmaceutically acceptable salts) and prodrugs, and prodrug salts of a compound of all the formulae disclosed in the present invention. The term also includes any solvates, hydrates, and polymorphs of any of the foregoing. The specific recitation of “stereoisomers,” “geometric isomers,” “tautomers,” “solvates,” “metabolites,” “salt” “prodrug,” “prodrug salt,” “conjugates,” “conjugates salt,” “solvate,” “hydrate,” or “polymorph” in certain aspects of the invention described in this application shall not be interpreted as an intended omission of these forms in other aspects of the invention where the term “compound” is used without recitation of these other forms.

The term “imine reactive reagent” refers to a reagent that is capable of reacting with an imine group. Examples of imine reactive reagent includes, but is not limited to, sulfites (H₂SO₃, H₂SO₂ or a salt of HSO₃⁻, SO₃²⁻ or HSO₂⁻ formed with a cation), metabisulfite (H₂S₂O₅ or a salt of S₂O₅²⁻ formed with a cation), mono, di, tri, and tetra-thiophosphates (PO₃SH₃, PO₂S₂H₃, POS₃H₃, PS₄H₃ or a salt of PO₃S³⁻, PO₂S₂³⁻, POS₃³⁻ or PS₄³⁻ formed with a cation), thio phosphate esters ((R₅O)₂PS(OR₅), R₅SH, R₅SOH, R₅SO₂H, R₅SO₃H), various amines (hydroxyl amine (NH₂OH), hydrazine (NH₂NH₂), NH₂OR₅, R₅NHR_{5′}, NH₂R₅), NH₂—CO—NH₂, NH2-C(═S)—NH₂), thiosulfate (H₂S₂O₃ or a salt of S₂O₃²⁻ formed with a cation), dithionite (H₂S₂O₄ or a salt of S₂O₄²⁻ formed with a cation), phosphorodithioate (P(═S)(OR₅)(SH)(OH) or a salt thereof formed with a cation), hydroxamic acid (R₅C(═O)NHOH or a salt formed with a cation), hydrazide (R₅CONHNH₂), formaldehyde sulfoxylate (HOCH₂SO₂H or a salt of HOCH₂SO₂⁻ formed with a cation, such as HOCH₂SO₂⁻Na⁺), glycated nucleotide (such as GDP-mannose), fludarabine or a mixture thereof, wherein R₅ and R_{5′} are each independently a linear or branched alkyl having 1 to 8 carbon atoms and are substituted with at least one substituent selected from —N(R₅)(R_{5′}), —CO₂H, —SO₃H, and —PO₃H; R₅ and R_{5′} can be further optionally substituted with a substituent for an alkyl described herein; Preferably, the cation is a monovalent cation, such as Na⁺ or K⁺. Preferably, the imine reactive reagent is selected from sulfites, hydroxyl amine, urea and hydrazine. More preferably, the imine reactive reagent is NaHSO₃ or KHSO₃.

“Cell binding agents” or “Cell binding molecules” may be of any kind presently known, or that become known, and include peptides and non-peptides. Generally, these can be antibodies (especially monoclonal antibodies) or a fragment of an antibody that contains at least one binding site, lymphokines, hormones, growth factors, nutrient-transport molecules (such as transferrin), or any other cell binding molecule or substance (such as vitamins).

More specific examples of cell binding agents that can be used include: monoclonal antibodies; single chain antibodies; fragments of antibodies such as Fab, Fab′, F(ab′)₂, F, {Parham, 131 J. Immunol. 2895-2902 (1983); Spring et al, 113 J. Immunol. 470-478 (1974); Nisonoff et al, 89 Arch. Biochem. Biophys. 230-244 (1960)}, fragments produced by a Fab expression library, anti-idiotypic (anti-Id) antibodies, CDR's, and epitope-binding fragments of any of the above which immunospecifically bind to cancer cell antigens, viral antigens or microbial antigens; interferons; peptides; lymphokines such as IL-2, IL-3, IL-4, IL-6; hormones such as insulin, TRH (thyrotropin releasing hormones), MSH (melanocyte-stimulating hormone), steroid hormones, such as androgens and estrogens; growth factors and colony-stimulating factors such as EGF, TGFα, insulin like growth factor (IGF-I, IGF-II) G-CSF, M-CSF and GM-CSF {Burgess, 5 Immunology Today 155-158 (1984)}; vitamins, such as folate and; transferrin {O'Keefe et al, 260 J. Biol. Chem. 932-937 (1985)}.

Monoclonal antibody technology permits the production of extremely selective cell binding agents in the form of specific monoclonal antibodies. Particularly well known in the art are techniques for creating monoclonal antibodies produced by immunizing mice, rats, hamsters or any other mammal with the antigen of interest such as the intact target cell, antigens isolated from the target cell, whole virus, attenuated whole virus, and viral proteins such as viral coat proteins. Selection of the appropriate cell binding agent is a matter of choice that depends upon the particular cell population that is to be targeted, but in general monoclonal antibodies are preferred if an appropriate one is available.

Cross-Linked PBDs and their Conjugates.

The cross-linked PBD dimer derivatives that have conjugated or can be conjugated to a cell-binding molecule for targeted treatment of cell proliferation, have the structure of Formula (I) as shown below:

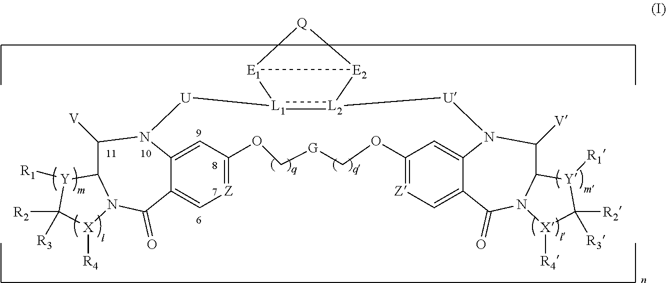

or their pharmaceutically acceptable salts, hydrates, or hydrated salts, or the polymorphic crystalline structures of these compounds or their optical isomers, racemates, diastereomers or enantiomers;

wherein:

----- represents an optional single bond or can be absent;

[symbol] represents an optional single bond or a double bond;

V and V′, the same or different, are independently selected from the group consisting of H, OH, —NHOH; OR₅ (an ether); OCOR₅ (an ester); OCOOR₅ (a carbonate); NR₅R₅′, NR₅COR₅′, or NR₅NR₅′NR₅″ (an amine); OCONR₅R₅′ (a carbamate); NR₅(C═NH)NR₅′R₅″ (a guanidinum); NR₅CONR₅′R₅″ (a urea); OCSNHR₅ (a thiocarbamate); —SH (a thiol); —SR₅ (a sulfide); SOR₅ a sulphoxide (a sulphoxide); SOOR₅ (a sulfone); SO₃, HSO₃, HS₂, or a salt of HSO³⁻, SO₃²⁻ or —HSO₂⁻ (a sulphite); OSO₃ (a bisulphite); NR₅SOOR₅′ (a sulfonamide); H₂S₂O₅ or a salt of S₂O₅²⁻ (a metabisulfite); PO₃SH₃, PO₂S₂H₂, POS₃H₂, PS₄H₂ or a salt of PO₃S³⁻, PO₂S₂³⁻, POS₃³⁻, PS₄³⁻ (mono-, di-, tri-, and tetra-thiophosphate); (R₅O)₂POSR₅′ (a thiophosphate ester); HS₂O₃ or a salt of S₂O₃²⁻ (a thiosulfate); HS₂O₄ or a salt of S₂O₄²⁻ (a dithionite); P(═S)(OR₅)(S)(OH) (a phosphorodithioate) or a salt thereof form with a cation; —NR₅OR₅′ (a hydroxylamine derivative); R₅C(═O)NOH (a hydroxamic acid) or a salt formed with a cation; HOCH₂SO₂⁻, or its salts (a formaldehyde sulfoxylate); NR₅COR_{5′} (an amide); O-glycoside; N₃ (an azido); CN (a cyano); X (a halo); C(R₅)(R_{5′})(R_{5″}) (a trialkyl), OP(O)(OR₅)(NHR_{5′}) or OP(O)(NHR₅)(NHR_{5′}) (a phosphoramidate (phosphoramidic acid), or P(R₅)(R_{5′})(R_{5″}) triarylphosphonium; Aa (an amino acid), or NR₅CO(Aa)ₜ (a peptide), wherein Aa is an amino acid or a polypeptide containing between t=1˜100 amino acid units; an aminoacid-derived group, such as an α-, β-, γ-, or ω-aminoacid, or an unnatural aminoacid; where R₅, R₅′ and R₅″ are described below;

l, m, q, l′, m′ and q′ are independently a number of 0, 1, 2, 3, 4, or 5; n is 1˜30;

X, X′, Y and Y′ the same or different, independently, represent N, O, S, an alkyl, such as CH₂ or CHR₅, an alkene, such as ═CH— or ═CR₅—, an ether, such as —C(OR₅)H—;

Z and Z′ the same or different, independently, represent N, CH, CR₅, COH, CNH₂, CNHR₅, or COR₅, or Z and Z′ link together with —COR₅OC—; wherein R₅ is independently selected from C₁˜C₈ alkyl and aryl;

G is —CH₂—, O, —N(R₅)—, S, —P(O)(OR₅)—, —P(O)(NR₅R_{5′})—,

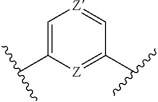

wherein Z and Z′ are defined above;

U and U′ are independently C(O), C(O)O, C(O)NH, C(O)N(R₅), C(═NH), C(═NH)O, C(═NH)NH, C(═NH)N(R₅), —C═N—, C(═S), C(O)S, C(S)NH, C(S)N(R₅), S(O), S(O)O, S(O)NH, S(O)(OR₅), S(O)(N(R₅)), S(O₂), S(O₂)O, P(O)(OR₅), P(O)(OR₅)O, P(O)(NH₂), P(O)(NR₅R_{5′}), P(O)(OR₅)NH—, P(O)(OR₅)NR_{5′}—, P(O)(N(R₅R_{5′})(N(R₅), P(S)(OR₅), P(S)(OR₅)O, P(S)(NH₂), P(S)(NR₅R_{5′}), P(S)(OR₅)NH—, P(S)(OR₅)NR_{5′}—, P(S)(N(R₅R_{5′})N(R₅), R₅, R₅O;

E₁ and E₂ are independently S, R₅S, C(O)S, C(O)NH, C(O)O, C(O)R₅S, C(═NH)NH, C(═NH)N(R₅), C(═NH)S, —C═N—, C(═S)S, C(O)S, C(═S)NH, C(═S)N(R₅), Ar—S, NC(O)CH₂S, ArC(O)CH₂S, S—S,

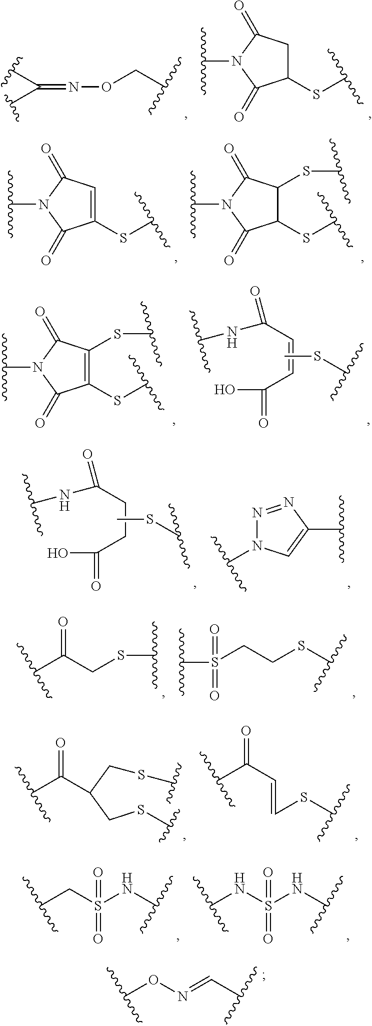

wherein the chemical bond in the middle of two atoms means it can link either adjoining two atoms;

L₁, and L₂ are independently a linker, or a linker which has a functional group on the linker that enables reaction with a cell-binding agent (CBA), Q. L₁, and L₂ are independently preferred a releasable linker, which has the formula of: -Ww-(Aa)r-Tt-; or -Ww-(Aa)r-Tt-Q; or Q-Ww-(Aa)r-Tt-; wherein: —W— is a Stretcher unit; w is 0 or 1; -Aa- is independently an Amino Acid unit; r is independently an integer ranging from 0 to 100; -T- is a Spacer unit, which can be a linear alkyl or branched alkyl, or polyethylene glycol spacer; and t is 0, or 1-100. The Stretcher unit W may independently contain a self-immolative spacer, peptidyl units, a hydrazone bond, a disulfide, an ester, or a thioether bond; w is 1 or 2 or 3; Preferably L₁, and L₂ are independently selected from O, NH, N, S, P, NNH, NHNH, N(R₃), N(R₃)N(R_{3′}), CH, CO, C(O)NH, C(O)O, NHC(O)NH, NHC(O)O, polyethyleneoxy unit of formula (OCH₂CH₂)ₚOR₃, or (OCH₂CH—(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 1000, or combination thereof; C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or (Aa)ᵣ, r=1-12 (one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;

R₁, R₂, R₃, R₄, R₁′, R₂′, R₃′, and R₄′ are the same or different and independently chosen from —H, an optionally substituted linear, branched or cyclic alkyl, alkenyl or alkynyl having from 1 to 10 carbon atoms, —(OCH₂CH₂)ₜR₅ (a polyethylene glycol unit), halogen, NH(C═NH)NH₂ (a guanidinium), —OR₅, —NR₅R₅′, —NO₂, —NCO, —NR₅COR₅′, —SR₅, —SOR₅ (a sulfoxide), —SO₂R₅ (a sulfone), —SO₃⁻M⁺ or —SO₃H (a sulfonate), —OSO₃⁻M⁺ or OSO₃H (a sulfate), —SO₂NR₅R₅′ (a sulfonamide), CN (a cyano), N₃ (an azido), —COR₅, —OCOR₅, —OCONR₅R₅′, CF₃, OR₅, Aryl, heterocycle, or P(O)R₅R₅′R₅″ and the linking group (L″) with the reactive group or a cell binding agent bonded thereto when Q, Q′ and T are absent;

R₅, R₅′ and R₅″ are independently selected from H, C₁˜C₈ of alkyl, alkenyl, alkinyl, heteroalkyl, aryl, arylalkyl, carbonyl, or pharmaceutical salts;

In addition, R₁ and R₂ join together, or R₁′ and R₂′ join together form a ═O (ketone), ═S, ═NR, —C(═O)R, or a double bond containing group ═CR₅R₅′; and R₁ and R₂ join together, or R₁′ and R₂′ join together, or R₃ and R₄ join together, or R₃′ and R₄′ join together form a C₃-C₁₂ aromatic, heterocyclic, or heteroaryl ring;

Q is a cell binding molecule, or a functional group that enables reaction with a cell-binding agent, or a functional group capable of reacting with a linker attached on a cell binding agent. The function group is chosen from a thiol, an amine, a hydrazine, an alkoxylamino, a disulfide substituent, a maleimido, a haloacetyl group, an N-hydroxy succinimide ester, a keton, an ester, an aldehyde, an alkynyl, an alkenyl, or protected thiol or disulfide group, such as SAc, SSR₁ or SSAr. Ar is aromatic group or hetero aromatic group. Q is preferably a cell-binding agent/molecule, selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and a binding peptide, protein, or small molecule attached on albumin, a polymer, a dendrimer, a liposome, a nanoparticle, a vesicle, or a (viral) capsid.

The term releasable linker refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with a intracellular thiol, such as the amillimolar range of abundant of glutathione inside the malignant cells.

The Stretcher unit (—W—), when present, may link a targeted binding molecular unit (CBA) to an amino acid unit (--Aa--), or links T when an Aa is not present. The Stretcher unit W may independently contain a self-immolative spacer, peptidyl units, a hydrazone bond, disulfide or thiolether bonds. In this regard a binding molecular (CBA) has a functional group that can form a bond with a functional group of a Stretcher. Useful functional groups that can be present on a binding molecule, either naturally or via chemical manipulation include, but are not limited to, sulfhydryl (—SH), amino, hydroxyl, oxyamino, alkynyl, heteroaromatic, carbonyl, the anomeric hydroxyl group of a carbohydrate, and carboxyl. Preferred functional groups are sulfhydryl, carboxy and amino. Sulfhydryl groups can be generated by reduction of an intramolecular disulfide bond of a Ligand (such as a protein or an antibody). Alternatively, sulfhydryl groups can be generated by reaction of an amino group of a lysine moiety of a cell-binding molecule using 2-iminothiolane (Traut's reagent) or thiolactone or another sulfhydryl generating reagent, such as modifies the cell-binding molecule with a disulfide bond linker, or a thiol ester following by reduction or hydrolysis respectively.

Preferably L₁, and L₂ are independently a chain of atoms selected from C, N, O, S, Si, and P, having 0˜500 atoms. The atoms used in forming the L₁, and L₂ may be combined in all chemically relevant ways, preferably are C₁-C₂₀ alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof. More preferably L₁, and L₂ are, the same or different, independently selected from O, NH, S, NHNH, N(R₃), N(R₃)N(R_{3′}), C₁-C₈ alkyl, amide, amines, imines, hydrazines, hydrazones; C₂-C₈ heteroalkyl, alkylcycloalkyl, ethers, esters, hydrazones, ureas, semicarbazides, carbazides, alkoxyamines, alkoxylamines, urethanes, amino acids, peptides, acyloxylamines, hydroxamic acids, or heterocycloalkyl; C₃-C₈ aryl, Ar-alkyl, heterocyclic, carbocyclic, cycloalkyl, heteroalkylcycloalkyl, alkylcarbonyl, or heteroaryl; polyethyleneoxy unit of formula (OCH₂CH₂)ₚOR₃, or (OCH₂CH(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 5000, or combination thereof; wherein R₃ and R_{3′} are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcyclo-alkyl, alkylcarbonyl, or heteroaryl; or C₂-C₈ esters, ether, or amide; or 1˜8 amino acids; or polyethyleneoxy having formula (OCH₂CH₂)ₚ or (OCH₂CH(CH₃))ₚ, wherein p is an integer from 0 to about 5000, or combination above thereof;

Optionally L₁ and L₂ may independently be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1˜8 natural or unnatural amino acid unites. The natural aminoacid is preferably selected from aspartic acid, glutamic acid, arginine, histidine, lysine, serine, threonine, asparagine, glutamine, cysteine, selenocysteine, tyrosine, phenylalanine, glycine, proline, tryptophan, alanine;

L₁ and L₂ may also independently contain a self-immolative or a non-self-immolative component, peptidic units, a hydrazone bond, a disulfide, an ester, an oxime, an amide, or a thioether bond. The self-immolative unit includes, but is not limited to, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals;

Preferably, the self-immolative linker component has one of the following structures:

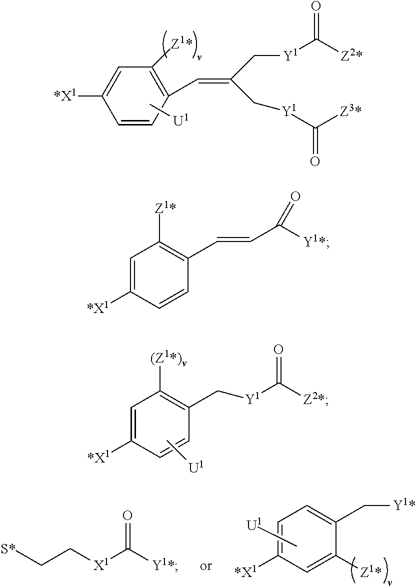

wherein the (*) atom is the point of attachment of additional spacer or releasable linker units, or the cytotoxic agent, and/or the binding molecule (CBA); X¹, Y¹, Z² and Z³ are independently NH, O, or S; Z¹ is H, OH, NHR₁, OR₁, SR₁, COX₁R₁, wherein X₁ and R₁ are defined above; v is 0 or 1; U¹ is independently H, OH, C₁˜C₆ alkyl, (OCH₂CH₂)ₙ, F, Cl, Br, I, OR₅, SR₅, NR₅R₅′, N═NR₅, N═R₅, NR₅R₅′, NO₂, SOR₅R₅′, SO₂R, SO₃R, OSO₃R₅, PR₅R₅′, POR₅R₅′, PO₂R₅R₅′, OPO(OR₅)(OR₅′), or OCH₂PO(OR₅(OR₅′), wherein R₅ and R₅′ are independently selected from H, C₁˜C₈ alkyl; C₂˜C₈ alkenyl, alkynyl, heteroalkyl, or amino acid; C₃˜C₈ aryl, heterocyclic, carbocyclic, cycloalkyl, heterocycloalkyl, heteroaralkyl, alkylcarbonyl, or glycoside; or pharmaceutical cation salts;

The non-self-immolative linker component is one of the following structures:

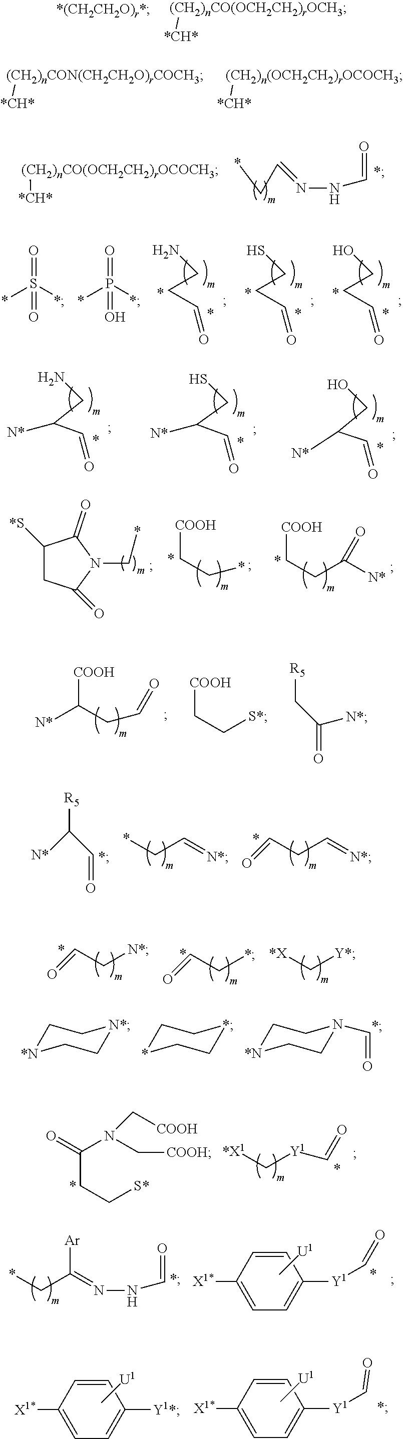

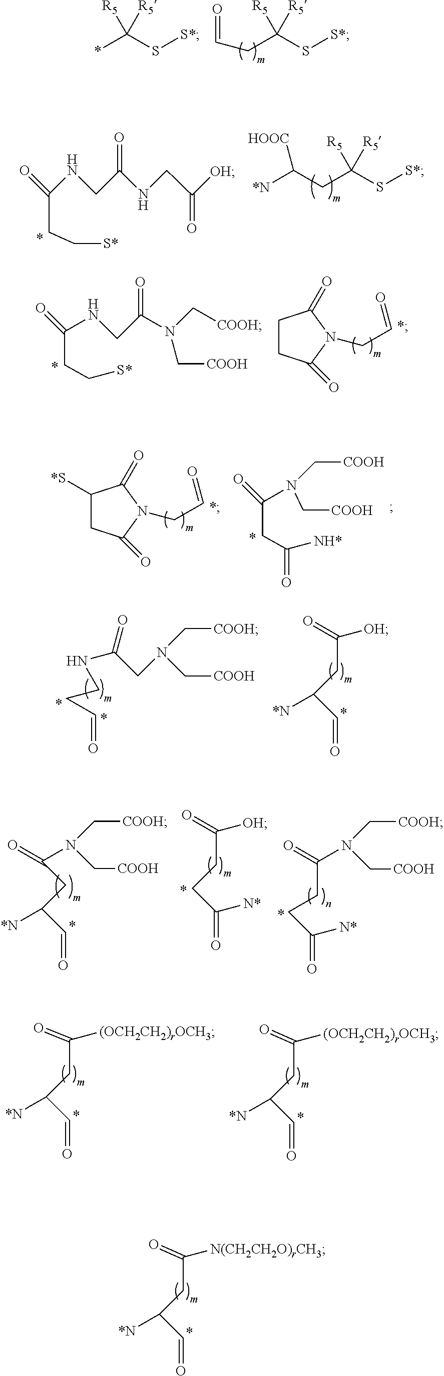

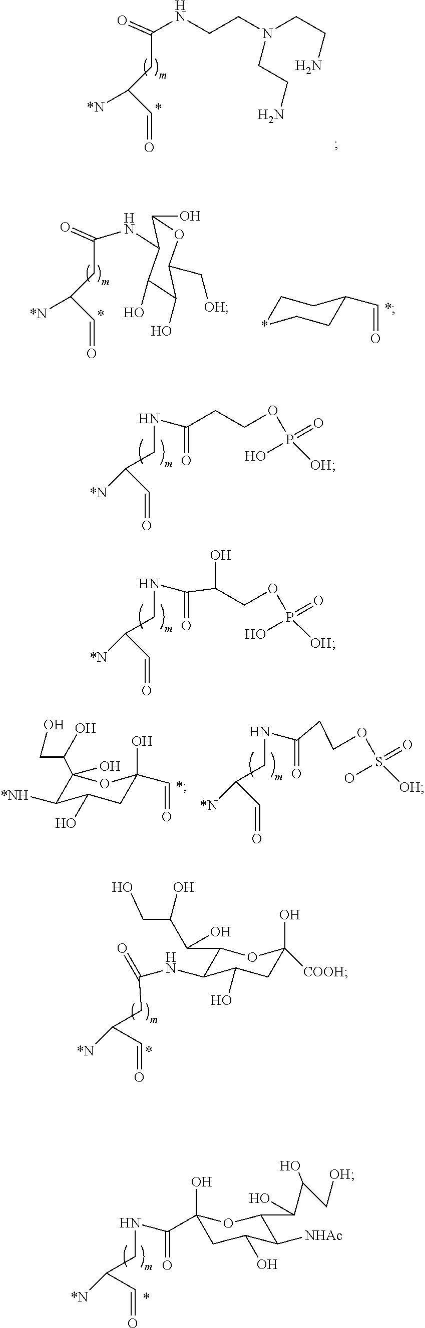

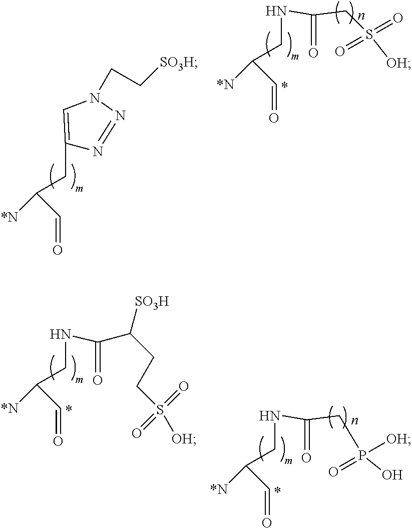

wherein the (*) atom is the point of attachment of additional spacer or releasable linkers, the cytotoxic agents, and/or the binding molecules; X¹, Y¹, U¹, R₅, R₅′ are defined as above; r is 0˜100; m and n are 0˜6 independently;

Further preferably, L₁ and L₂ may independently be a releasable linker. The term releasable linker refers to a linker that includes at least one bond that can be broken under physiological conditions, such as a pH-labile, acid-labile, base-labile, oxidatively labile, metabolically labile, biochemically labile or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with a intracellular thiol, such as a millimolar range of abundant of glutathione inside the malignant cells;

Examples of the releasable linkers L₁ or L₂ include, but not limited: —(CR₅R(O)ₘ(Aa)ᵣ(CR₇R₈)ₙ(OCH₂CH₂)ₜ—, —(CR₅R₆)ₘ(CR₇R₈)ₙ(Aa)ᵣ(OCH₂CH₂)ₜ—, -(Aa)ᵣ-(CR₅R₆)ₘ(CR₇R₈)ₙ(OCH₂CH₂)ₜ—, —(CR₅R₆)ₘ(CR₇R₈)ₙ(OCH₂CH₂)ᵣ(Aa)ₜ-, —(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)ₜ(NR₁₁CO)(CR₉R₁₀)ₙ(OCH₂CH₂)ᵣ—,—(CR₅R₆)ₘ(OCO)(Aa)ₜ(CR₉R₁₀)ₙ—(OCH₂CH₂)ᵣ—, —(CR₅R(O)ₘ(OCNR₇)(Aa)ₜ(CR₉R₁₀)ₙ(OCH₂CH₂)ᵣ—, —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ—(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ(CR₉R₁₀)ₙ—(OCH₂CH₂)ᵣ—, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)ₜ(CR₇R₈)ₙ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)ₜ-(CR₇R₈)ₙ—, —(CR₅R₆)ₜ—N-methylpiperazin-CO(Aa)ₜ-(CR₇R₈)ₙ—, —(CR₅R)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ₜ-, —(CR₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)ᵣ(CR₇R₈)ₙ(OCH₂CH₂)ₜ—, —K(CR₅R₆)ₘ(CR₇R₈)ₙ(Aa)ᵣ(OCH₂CH₂)ₜ—, —K(Aa)ᵣ-(CR₅R₆)ₘ(CR₇R₈)ₙ(OCH₂CH₂)ₜ—, —K(CR₅R₆)ₘ(CR₇R₈)ₙ(OCH₂CH₂)ᵣ(Aa)ₜ-, —K(CR₅R₆)ₘ—(CR₇═CR₈)(CR₉R₁₀)ₙ(Aa)ₜ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R(O)ₘ(Aa)ₜ(NR₁₁CO)(CR₉R₁₀)ₙ(OCH₂CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OCH₂CH₂)ᵣ—, —K(CR5R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)ₜimidazolyl-CO—(CR₇R₈)ₙ—, —K(CR₅R₆)ₜmorpholino-CO(Aa)ₜ(CR₇R₈)ₙ—, —K(CR₅R₆)ₜpiperazino-CO(Aa)ₜ-(CR₇R₈)ₙ—, —K(CR₅R₆)ₜ—N-methylpiperazinCO(Aa)ₜ(CR₇R₈)ₙ—, —K(CR₅R)ₘ(Aa)ₜphenyl, —K—(CR₅R₆)ₘ-(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ₜ-; wherein m, Aa, m, and n are described above; t and r are 0-100 independently; R₃, R₄, R₅, R₆, R₇, and R₈ are independently chosen from H; halide; C₁˜C₈ alkyl; C₂˜C₈ aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—N—, O, S, Se, B, Het (heterocyclic or heteroaromatic ring having C₃-C₈), or peptides containing 1-20 of the same or different amino acids;

Additionally U, U′, E, E′, L₁ and L₂ may independently contain one or more units of the following hydrophilic structures:

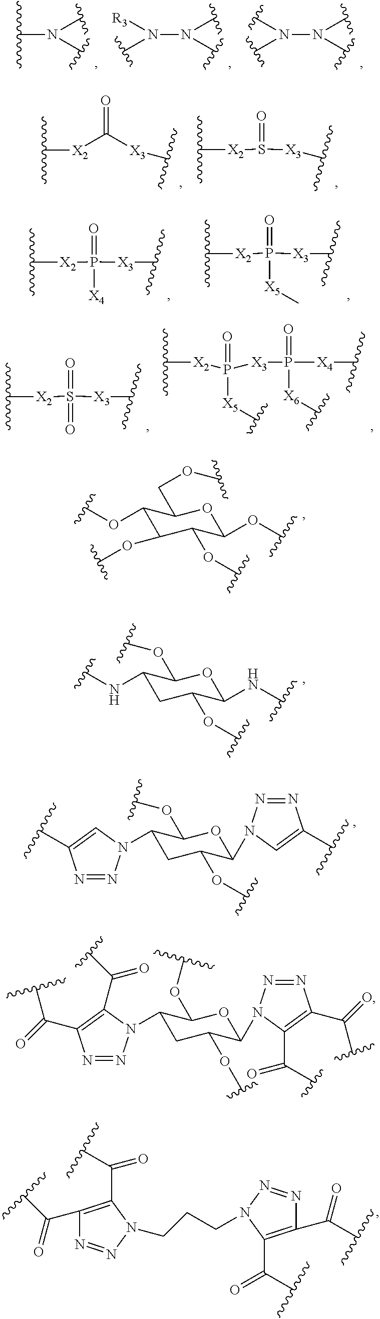

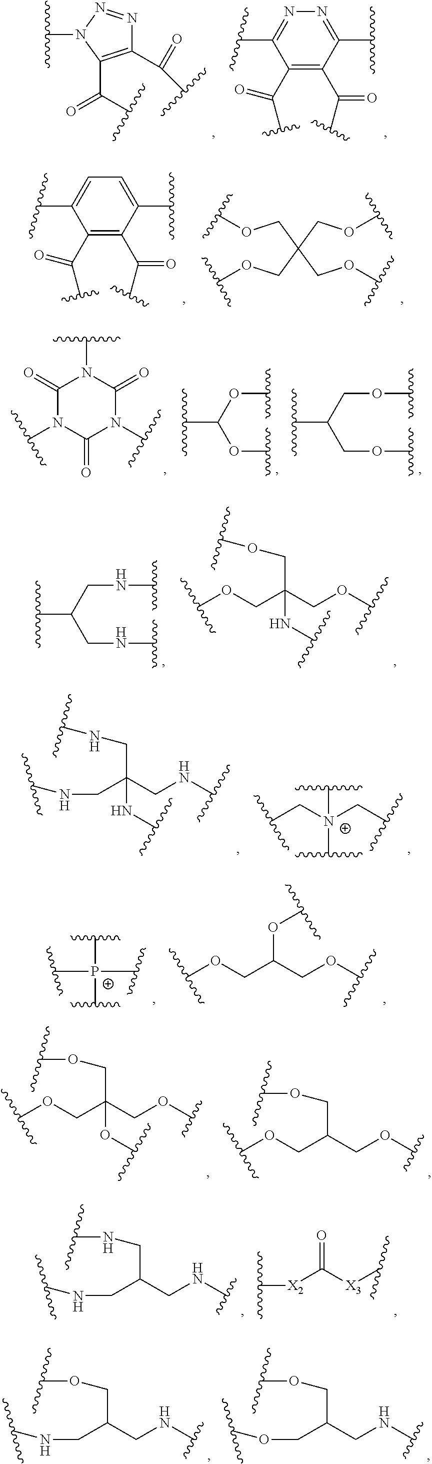

wherein [symbol] is the site of linkage; X₂, X₃, X₄, X₅, or X₆, are independently selected from NH; NHNH; N(R₃); N(R₃)N(R_{3′}); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or 1˜8 amino acids; wherein R₃ and R_{3′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ esters, ether, or amide; or polyethyleneoxy unit having formula (OCH₂CH₂)ₚ or (OCH₂CH(CH₃))ₚ, wherein p is an integer from 0 to about 5000, or combination above thereof;

More preferably, L₁, or L₂, are independently linear alkyl having from 1-6 carbon atoms, or polyethyleneoxy unit having formula (OCH₂CH₂)ₚ, p=1˜5000, or a peptide containing 1˜4 units of aminoacids (L or D form), or combination above.

In addition, U, U′, L₁, L₂, L′, E₁ or E₂ may independently be composed of one or more following components as shown below:

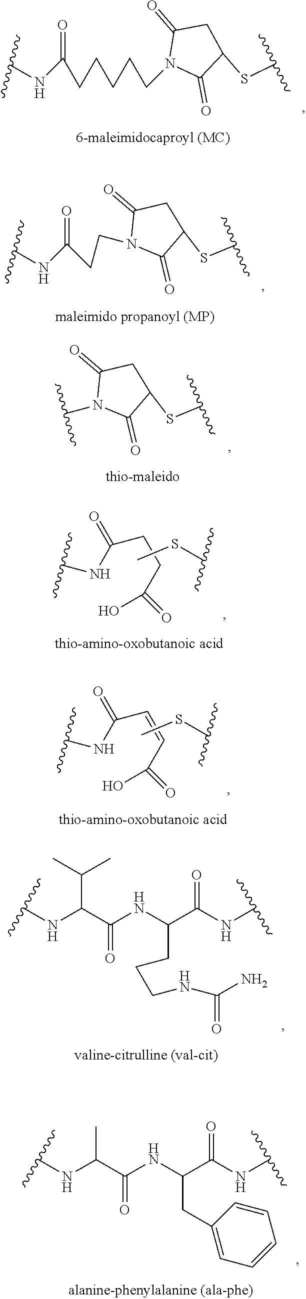

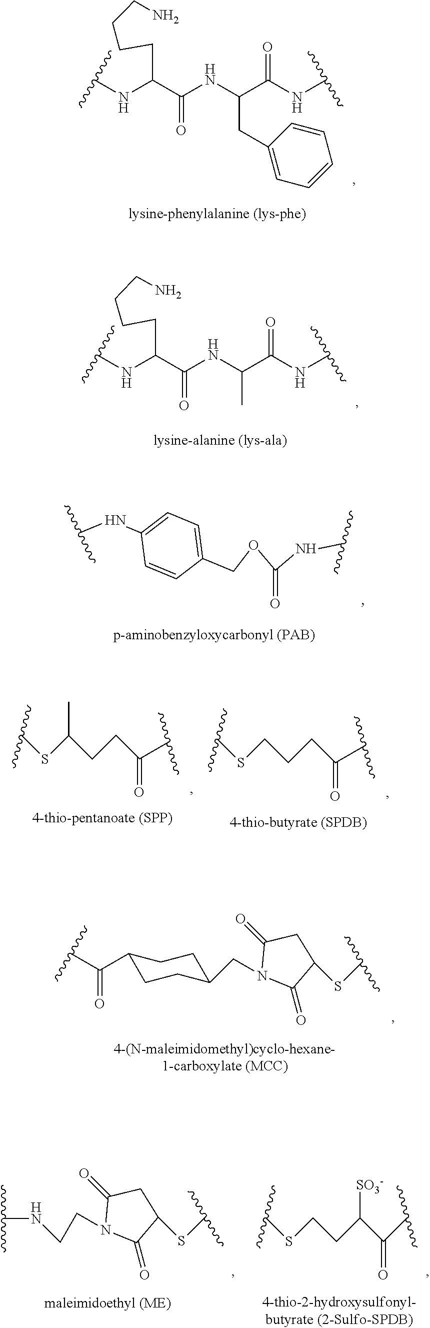

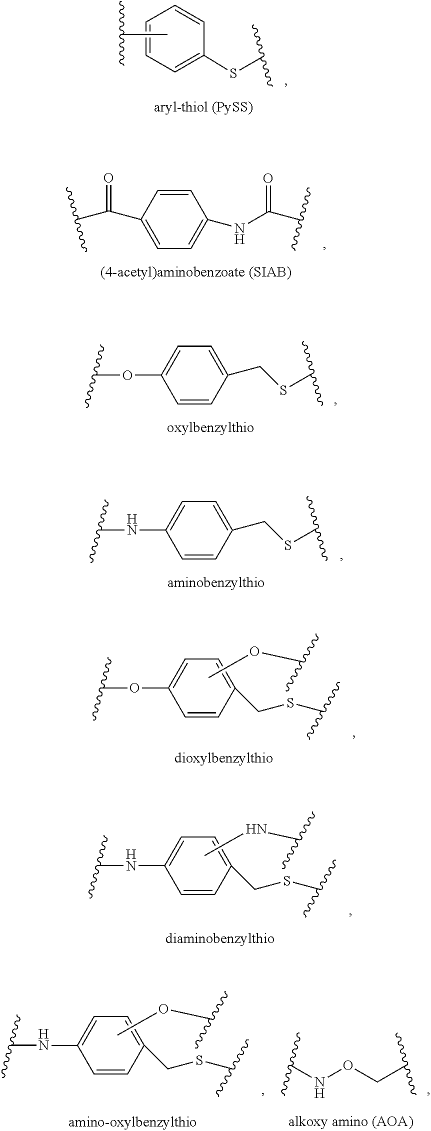

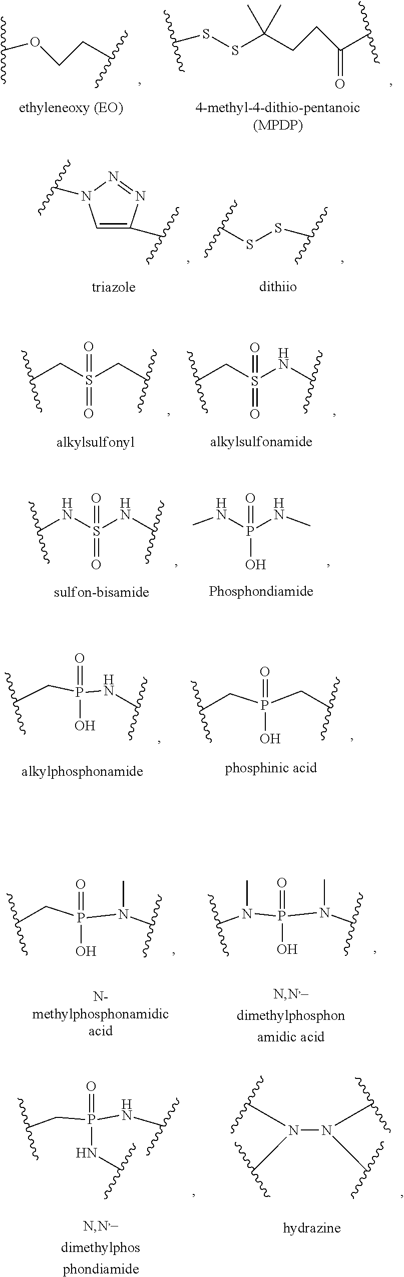

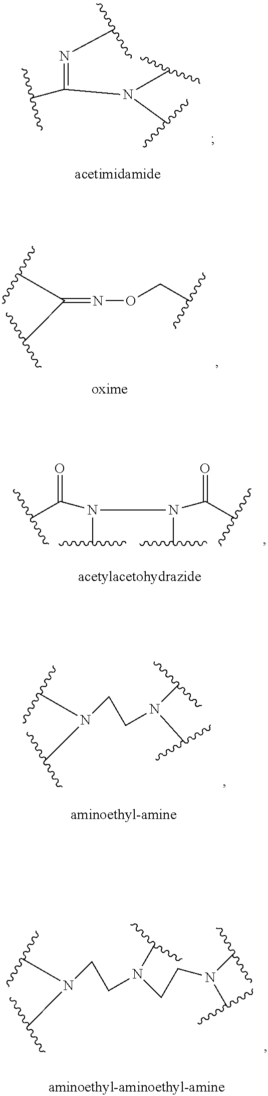 [SMILES: */N=C(/*)N(*)* ; *CON=C(*)* ; *C(=O)N(*)N(*)C(*)=O ; *N(*)CCN(*)* ; *N(*)CCN(*)CCN(*)*]

L- or D-, natural or unnatural peptides containing 1-20 amino acids; wherein a connecting bond in the middle of atoms means that it can connect either neighbor carbon atom bonds; wavery line is the site wherein another bond can be connected to;

Alternatively, U, U′, E₁, or E₂, can be independently absent.

The compounds of the general Formula (I) having geometrical and stereoisomers are also a part of the invention.

A preferred stereoisomer of the Formula (I) is presented by the following Formula (Ia), (Ib), (Ic), (Ie):

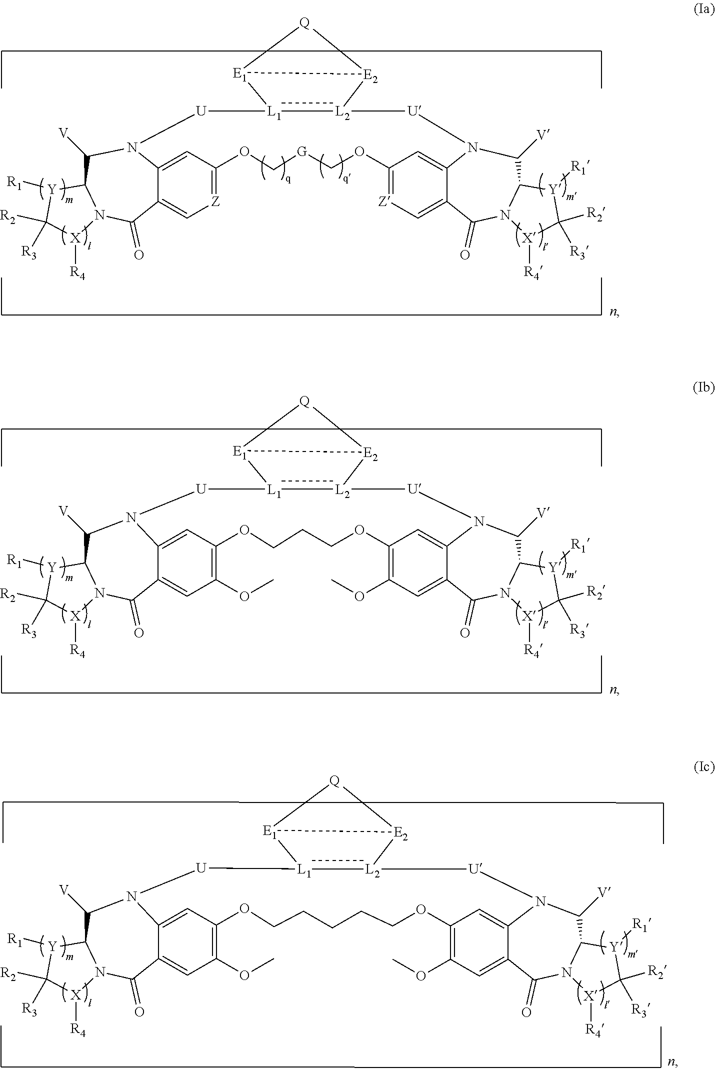

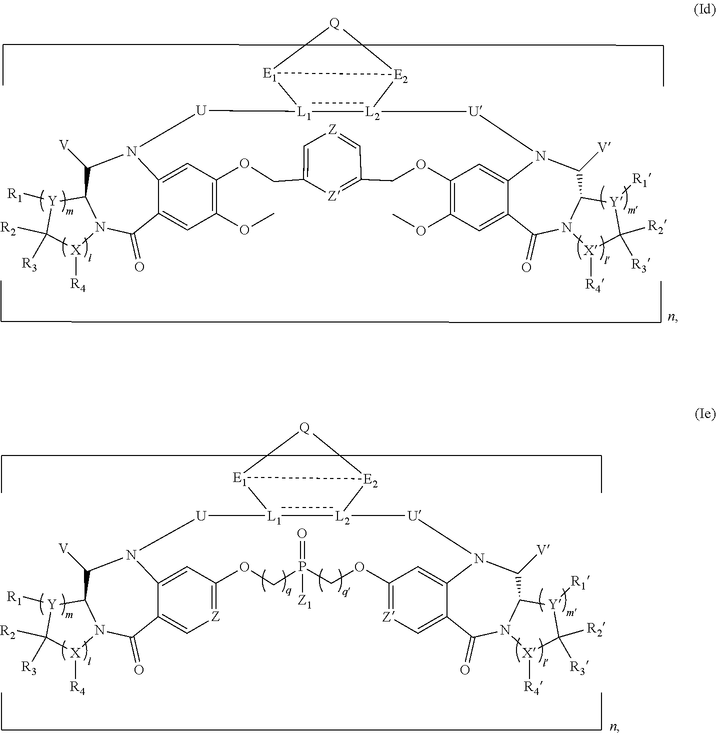

wherein Z₁ is OH, NH₂, OR₁, NHR₁, NR₁R₂, SR₁, NHR₁COX₁R₁, OR₁COX₁R₁, or N(R₂)R₁COX₁R₁; [symbol] , -----, X, X′, Y, Y′, Z, Z′, l, l′, m, m′, n, q, q′, R₁, R₁′, R₂, R₂′, R₃, R₃′, R₄, R₄′, V, V′, U, U′ L₁c, L₂, E₁, E₂ and Q are the same as in Formula (I).

In more preferred embodiment according to Formula (I), the conjugate of cross-linked PBD dimer derivatives of the invention have the Formula (I-01)˜(I-18) below:

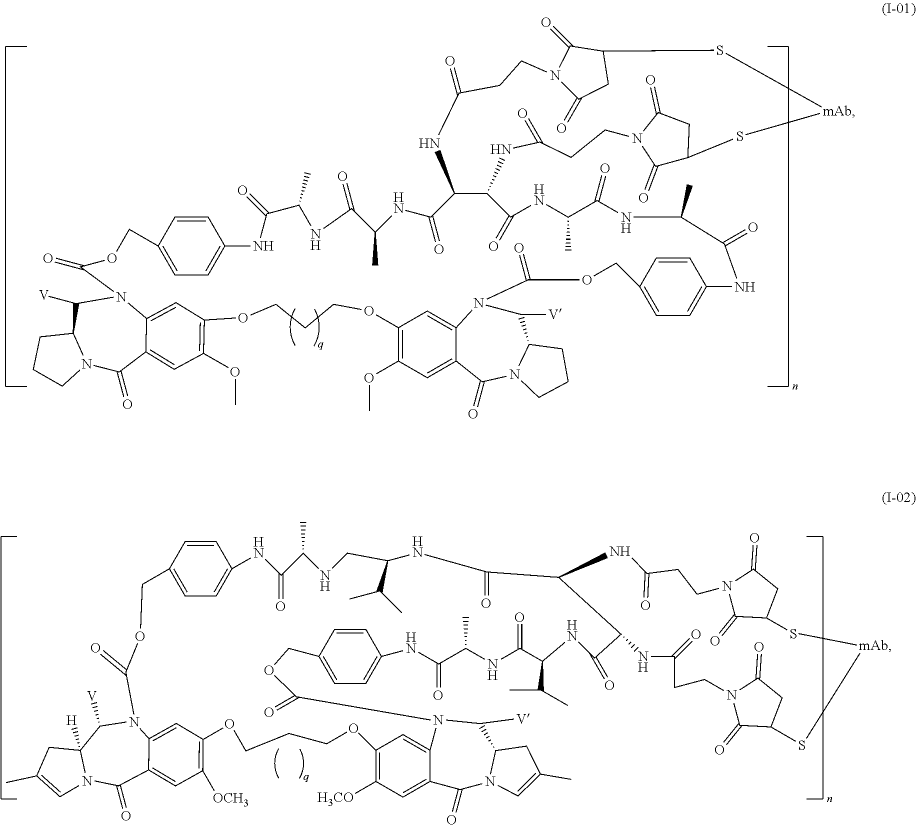

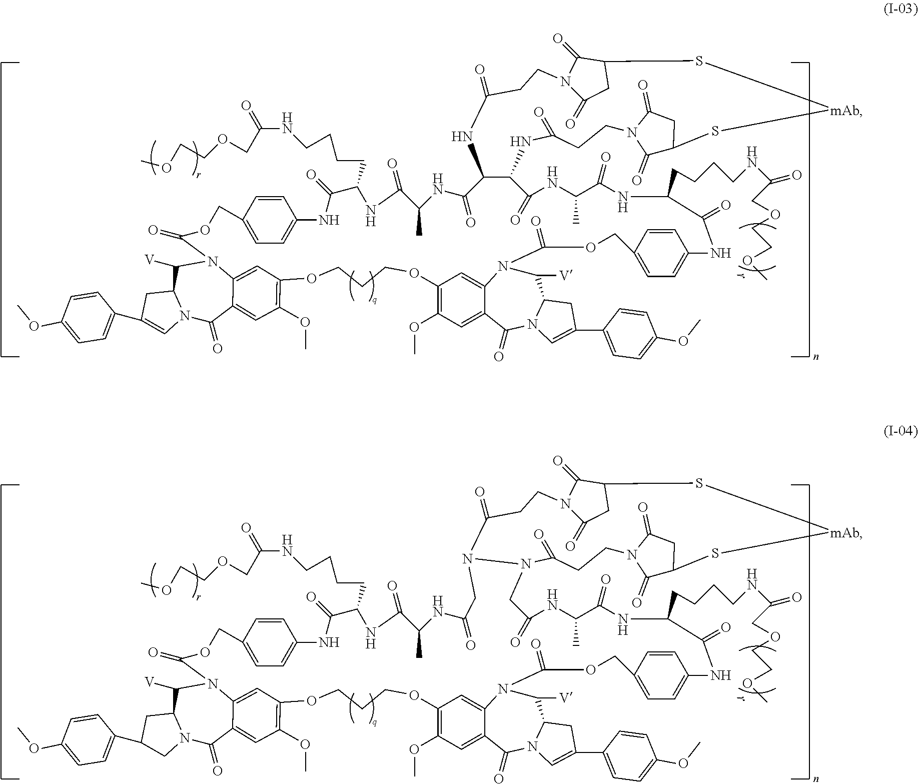

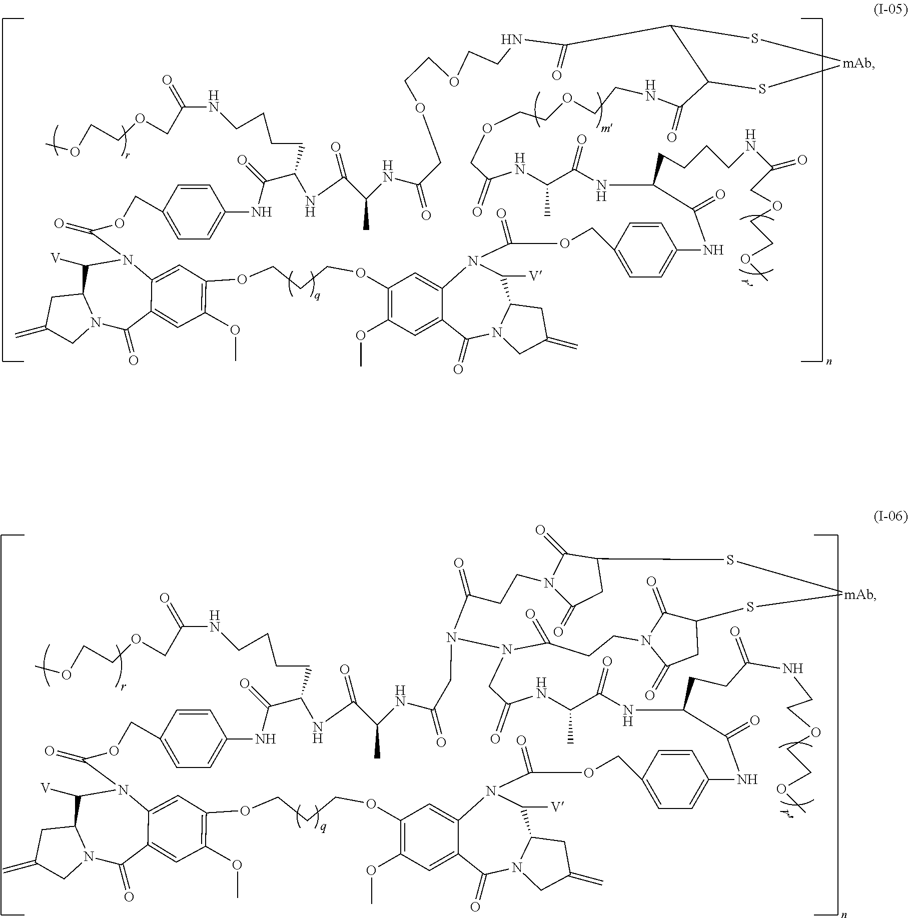

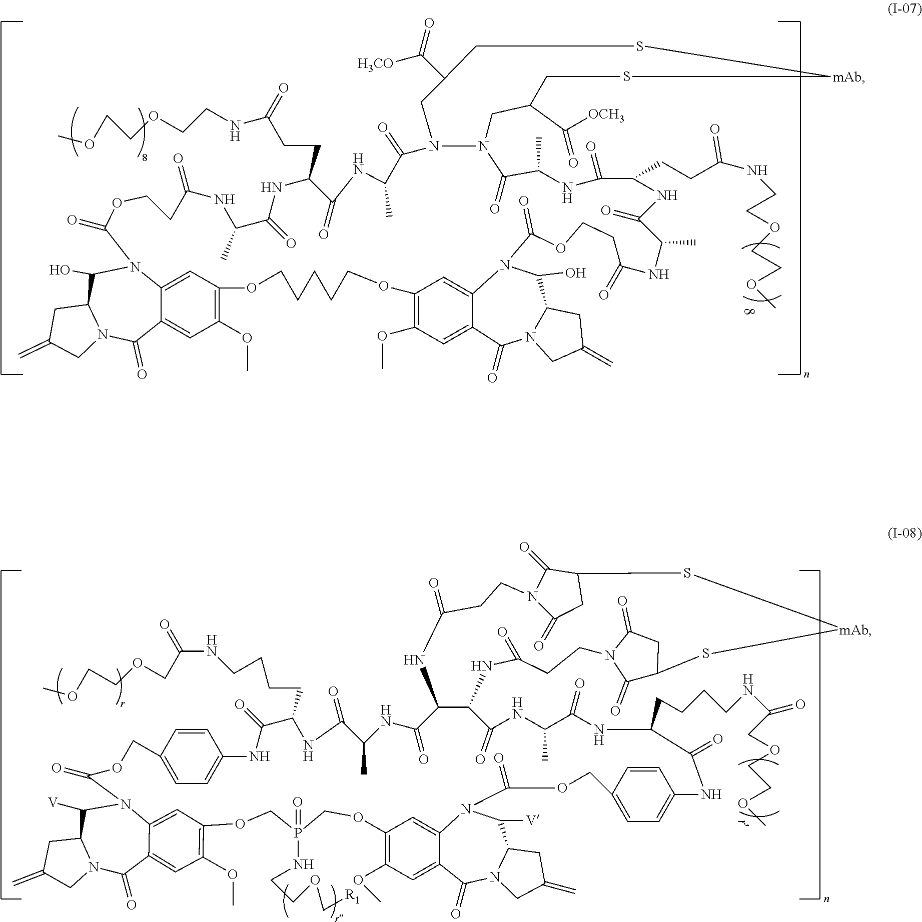

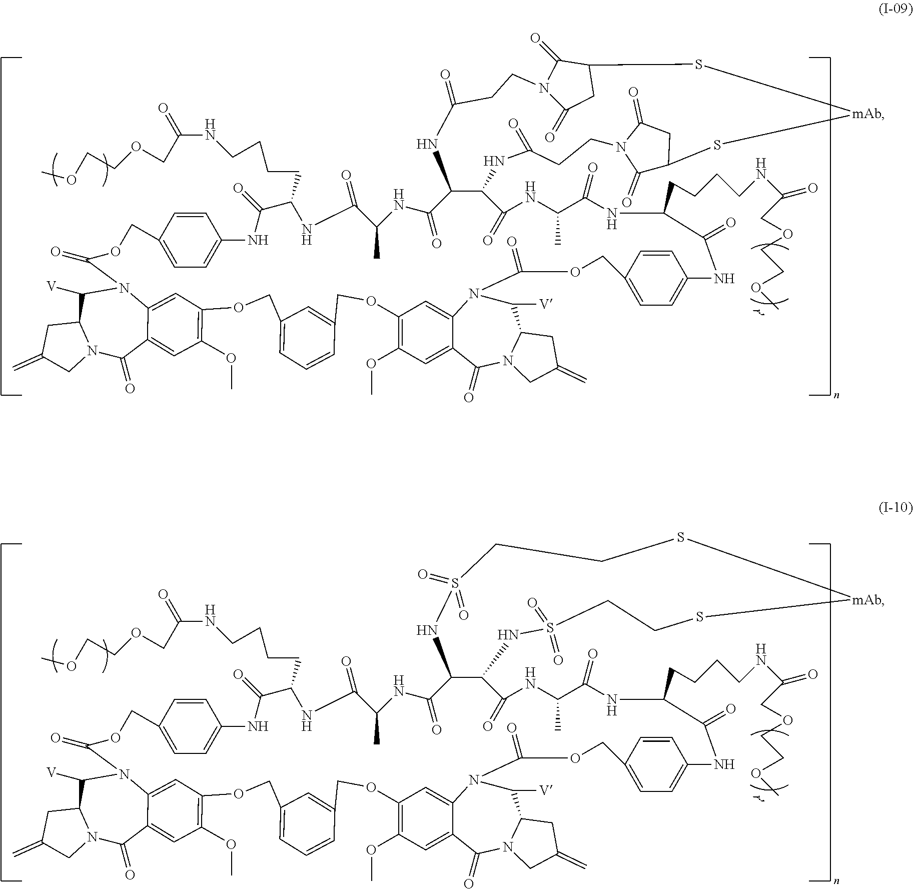

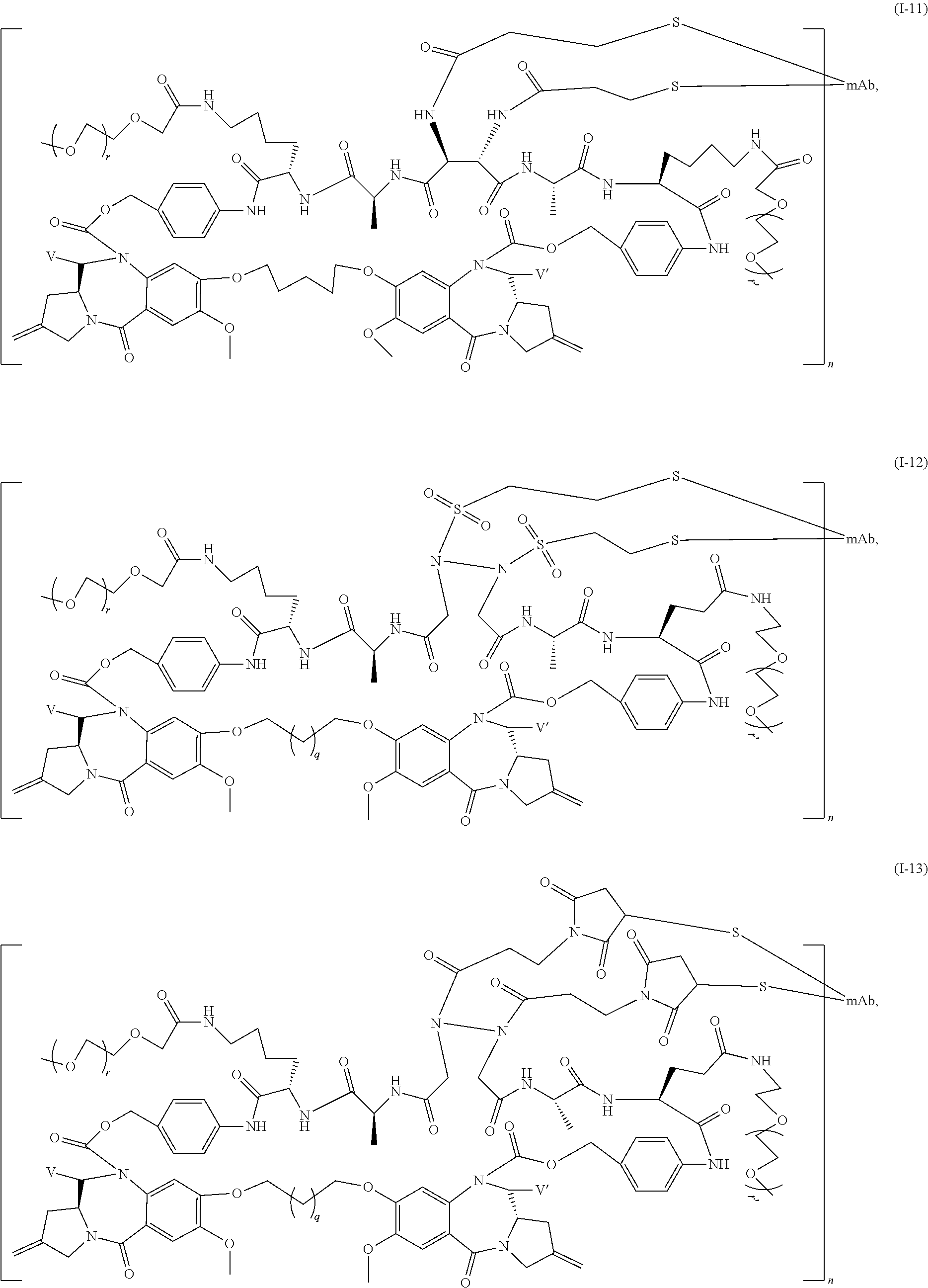

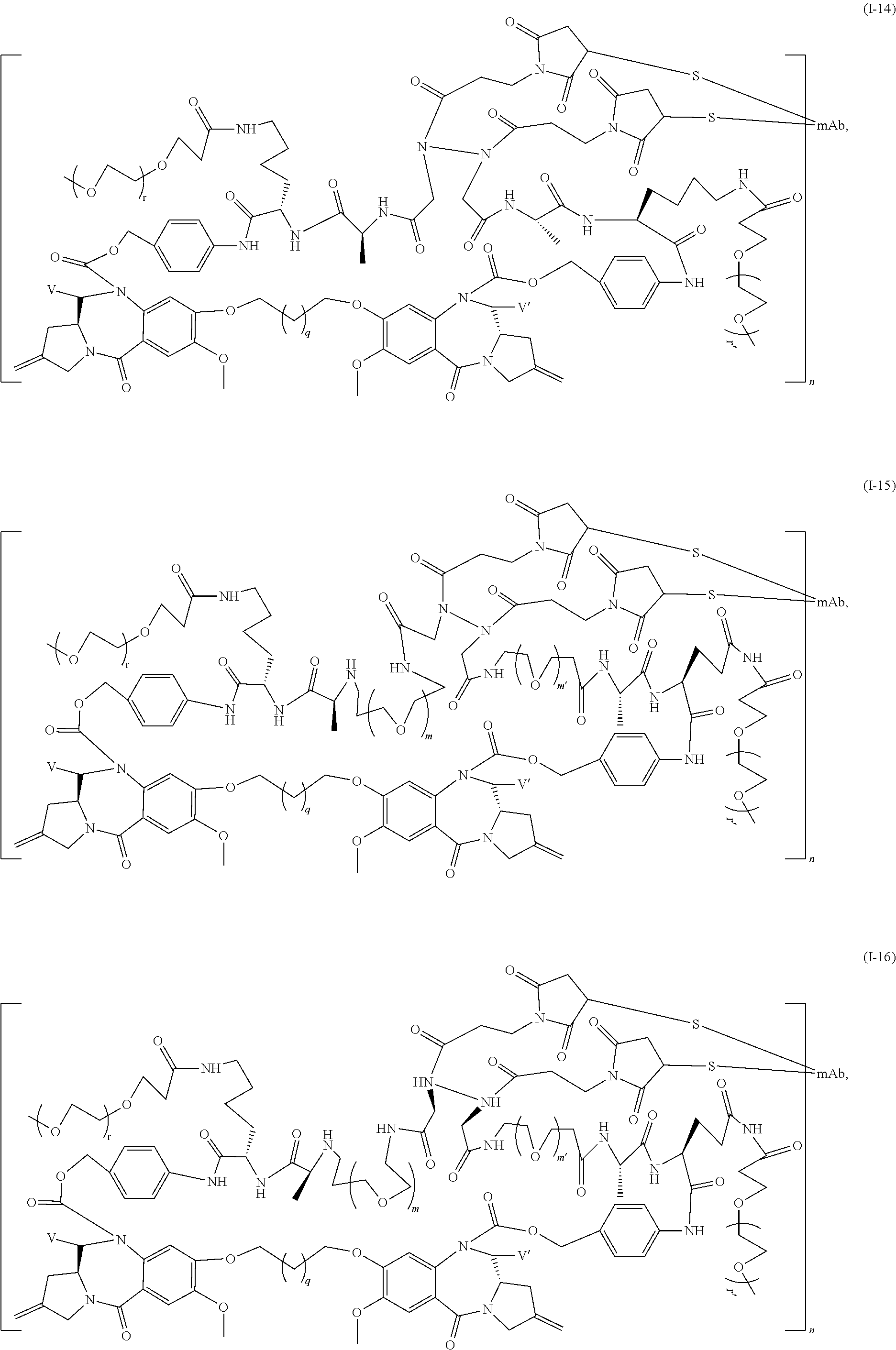

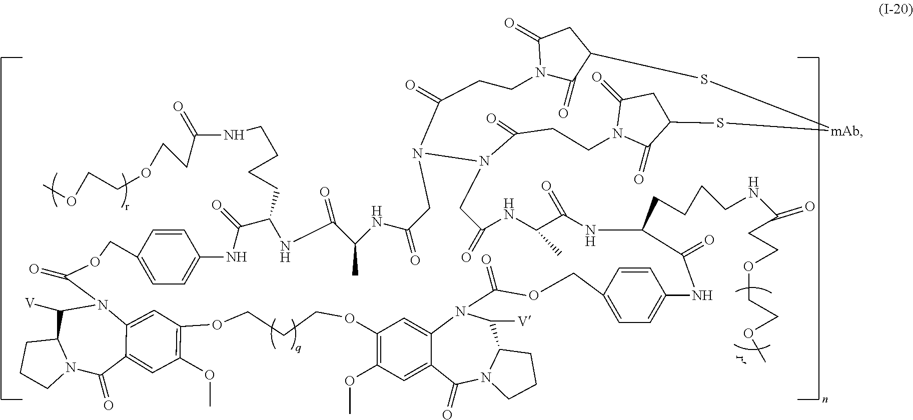

wherein V, V′, n and q are defined the same above; mAb is a cell-binding molecule, preferably an antibody; r, r′ and r″ are independently 0-200.

In another embodiment, the present invention discloses mono-linkage of conjugation of PBDs derivatives to a cell binding molecule, as shown in Formula (II), (III) and (IV), for targeted treatment of cell proliferation:

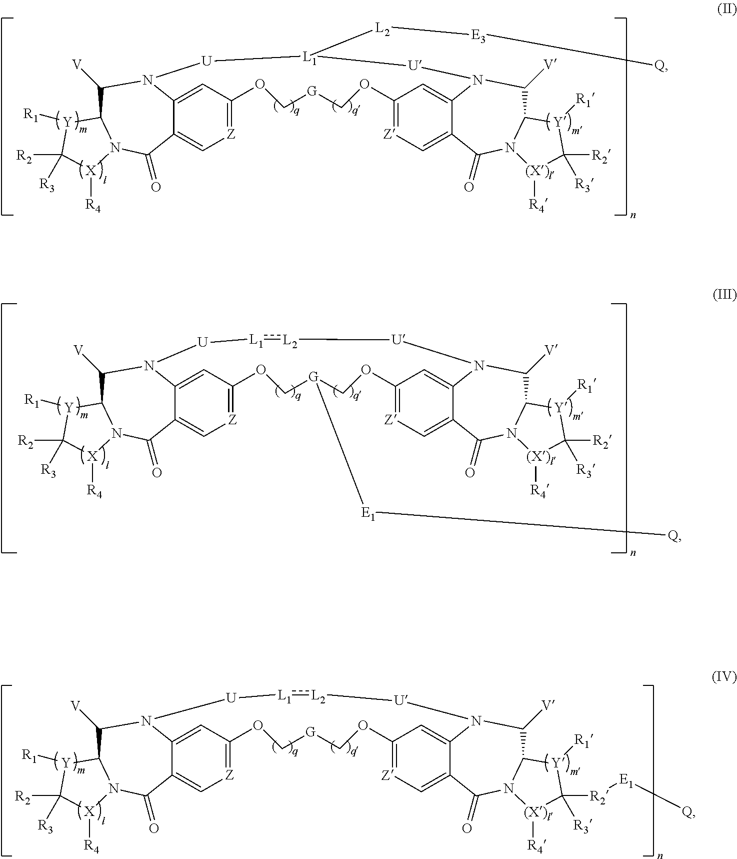

wherein [symbol] , ------, X, X′, Y, Y′, Z, Z′, l, l′, m, m′, n, q, q′, R₁, R₁′, R₂, R₂′, R₃, R₃′, R₄, R₄′, V, V′, U, U′ L₁, L₂, G, Q, E₁, and E₂ are the same defined as in Formula (I).

In preferred embodiment according to Formula (II), (III) and (IV), the conjugates of PBD derivatives of the invention having the Formula (II-01)˜(II-11), (III-01)˜(III-06) and (IV-01)˜(IV-11) below:

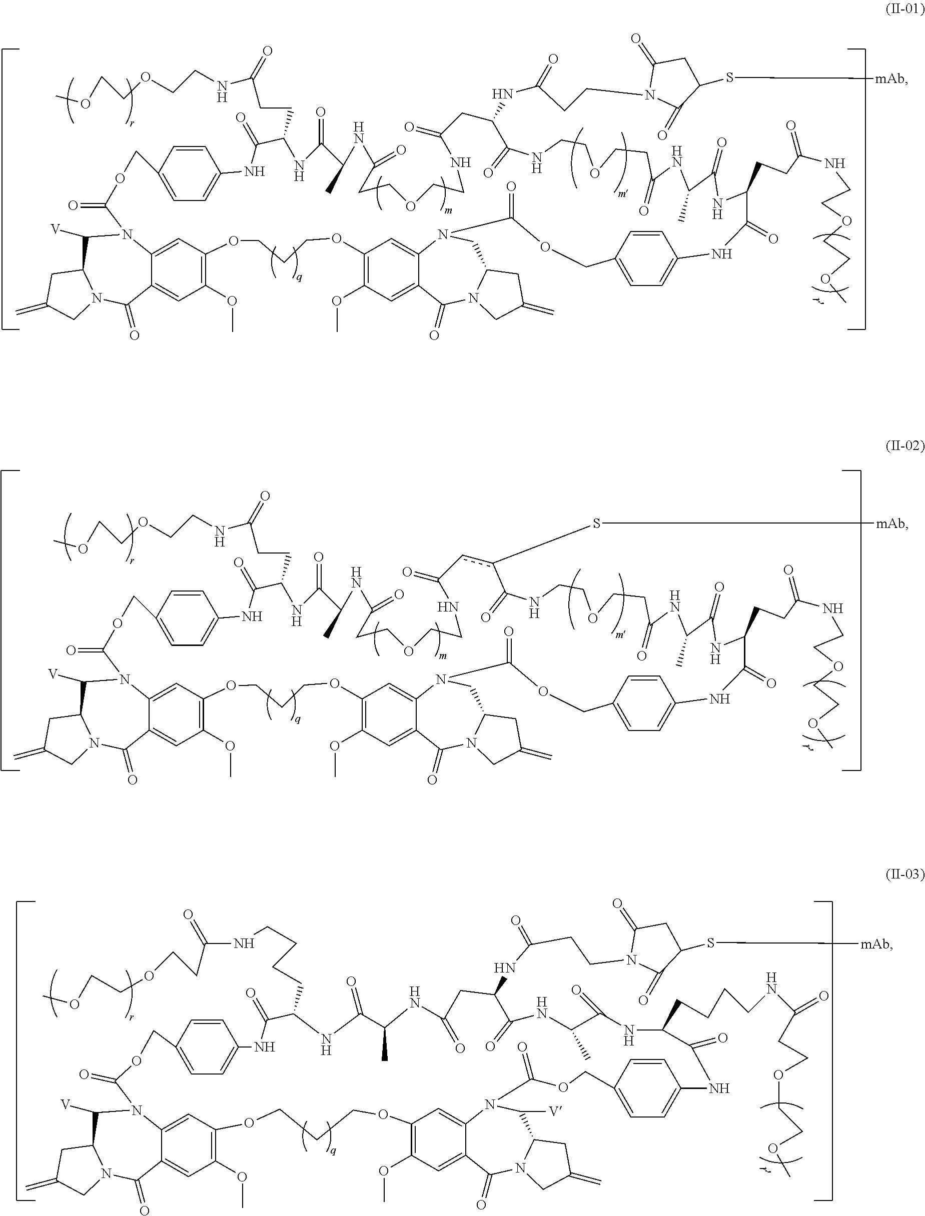

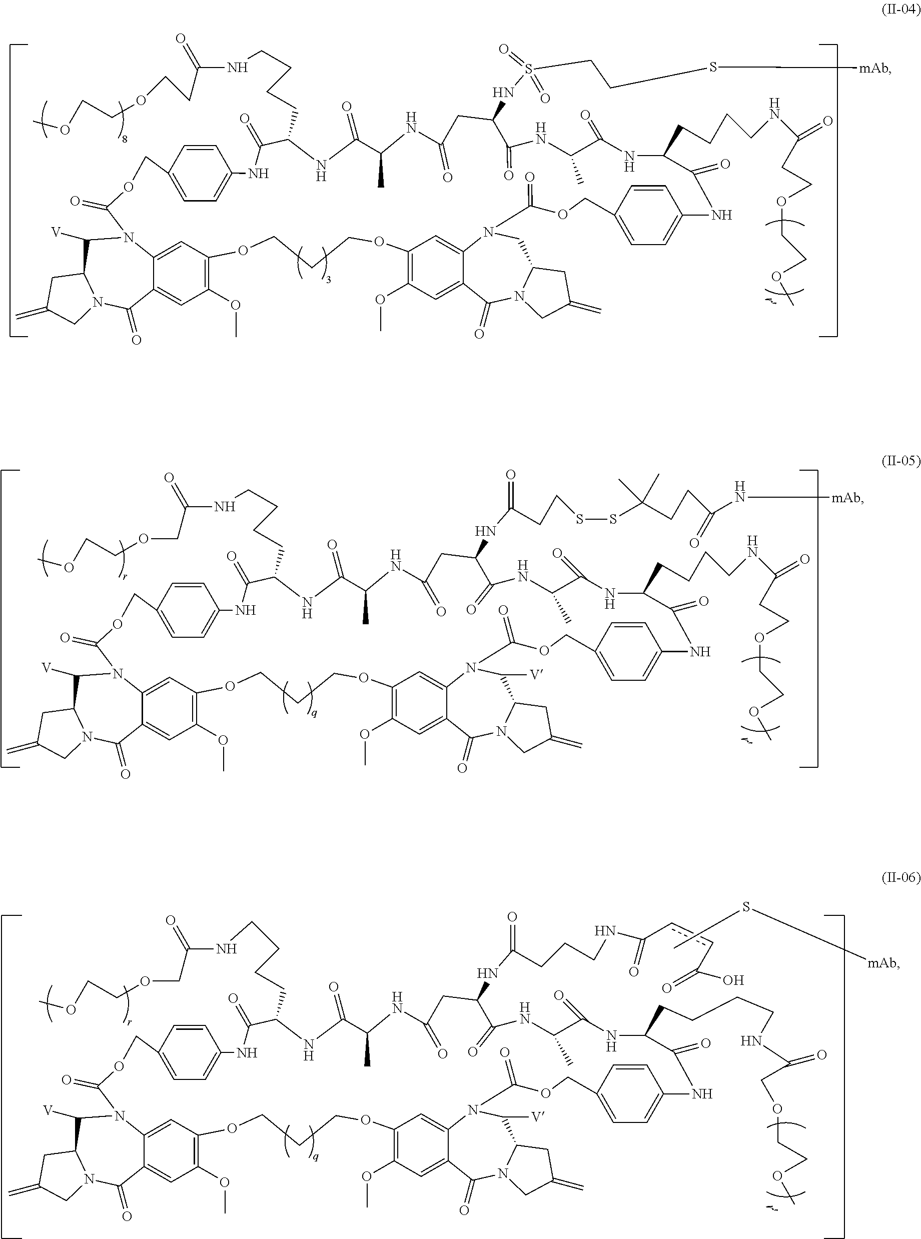

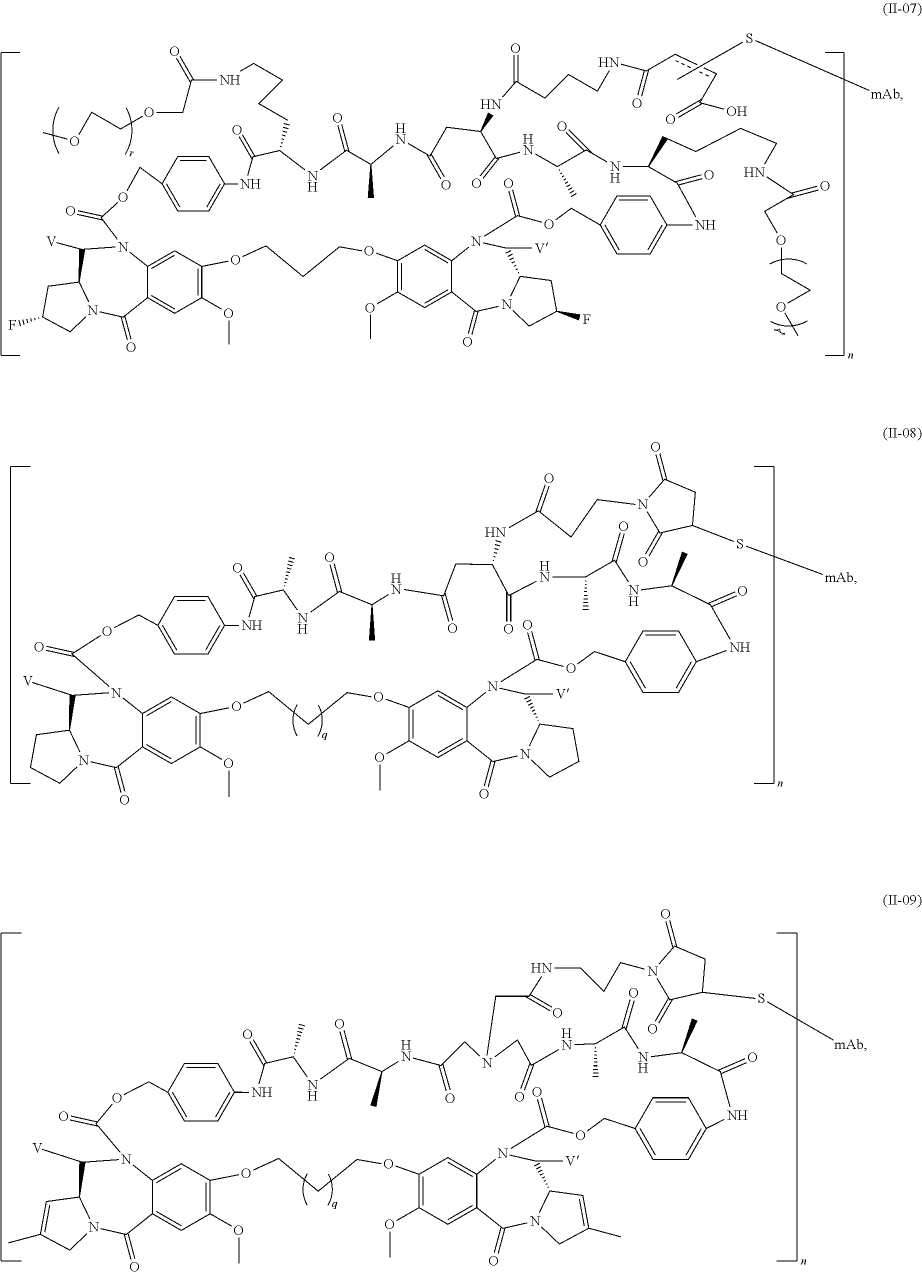

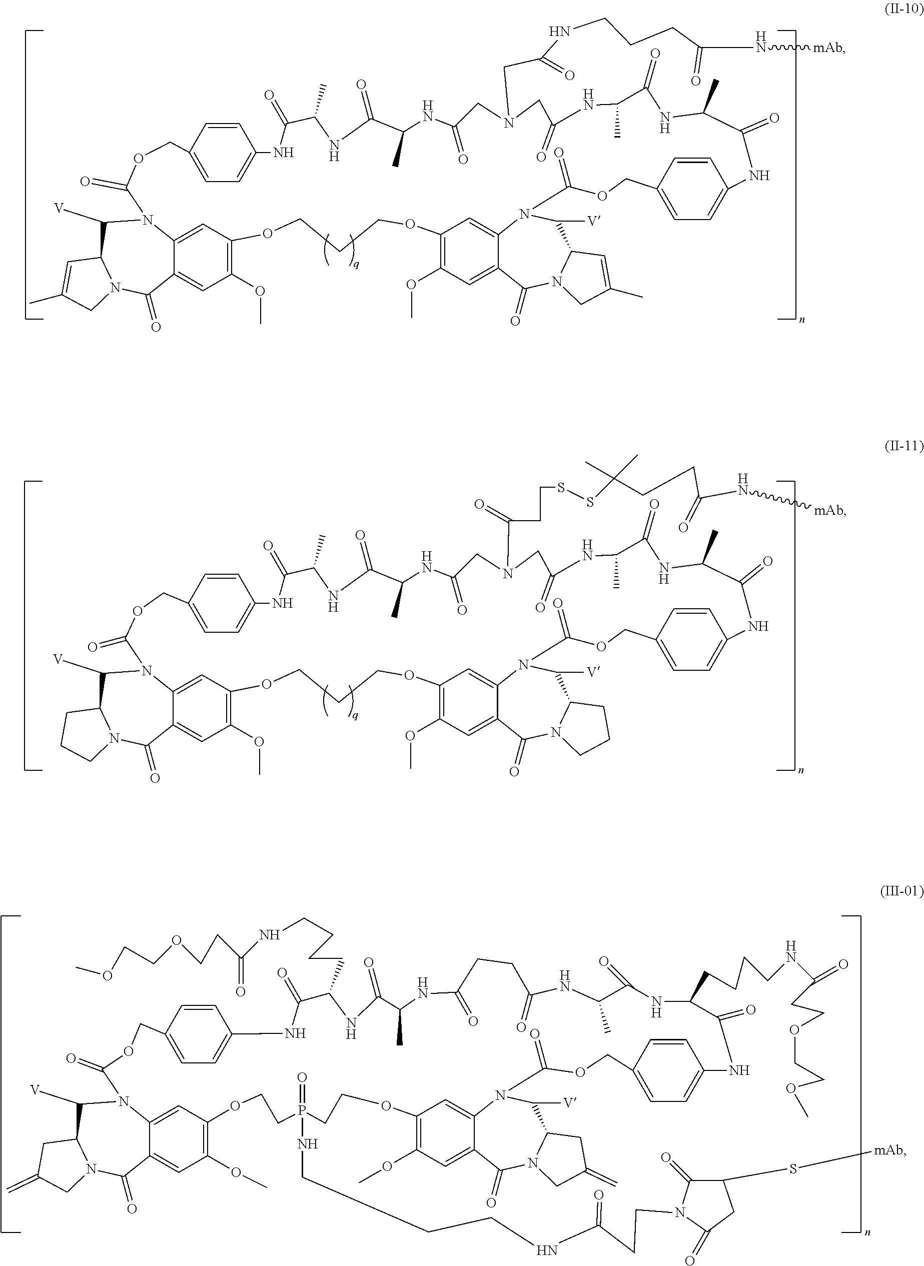

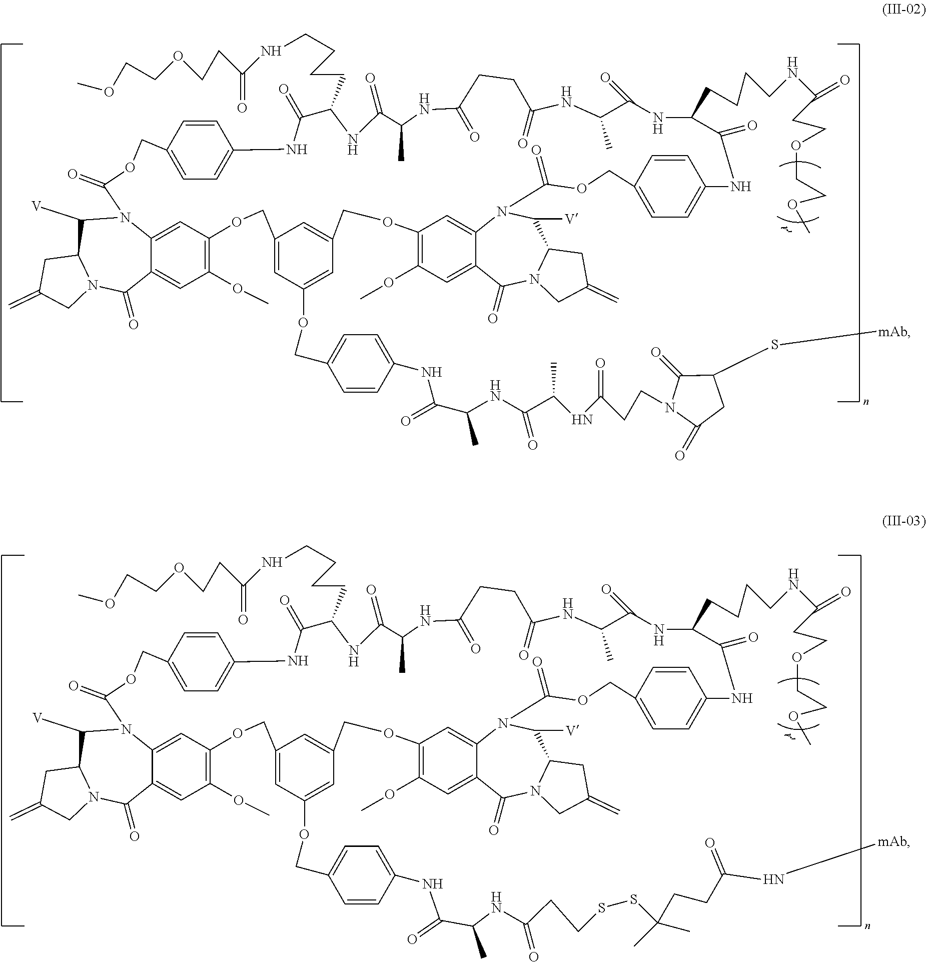

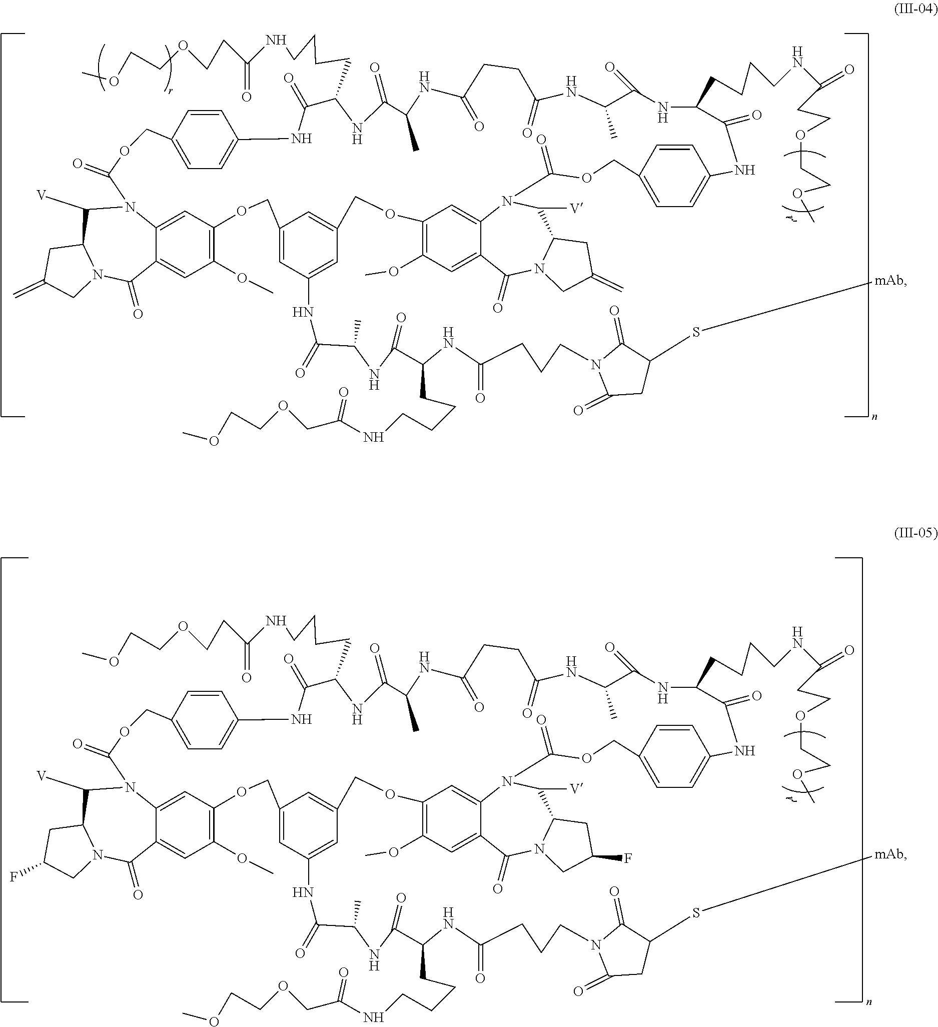

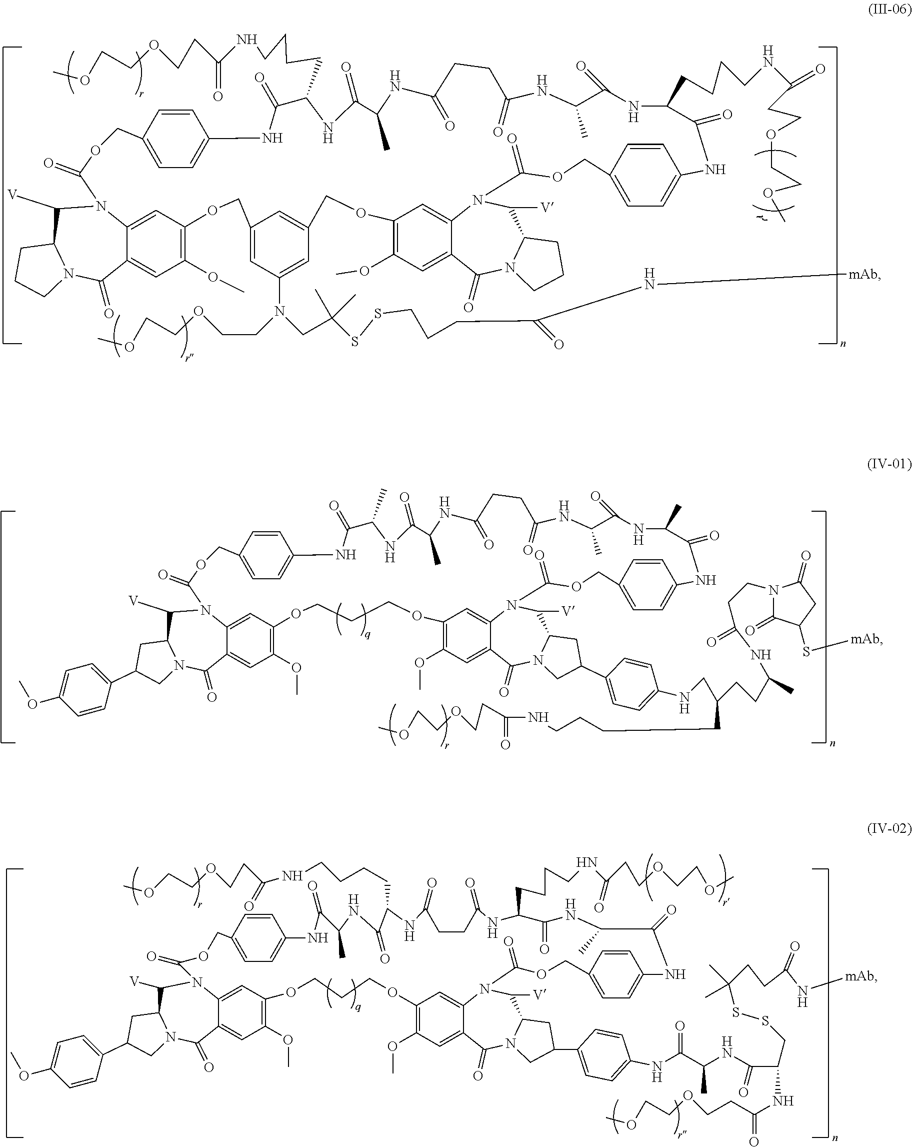

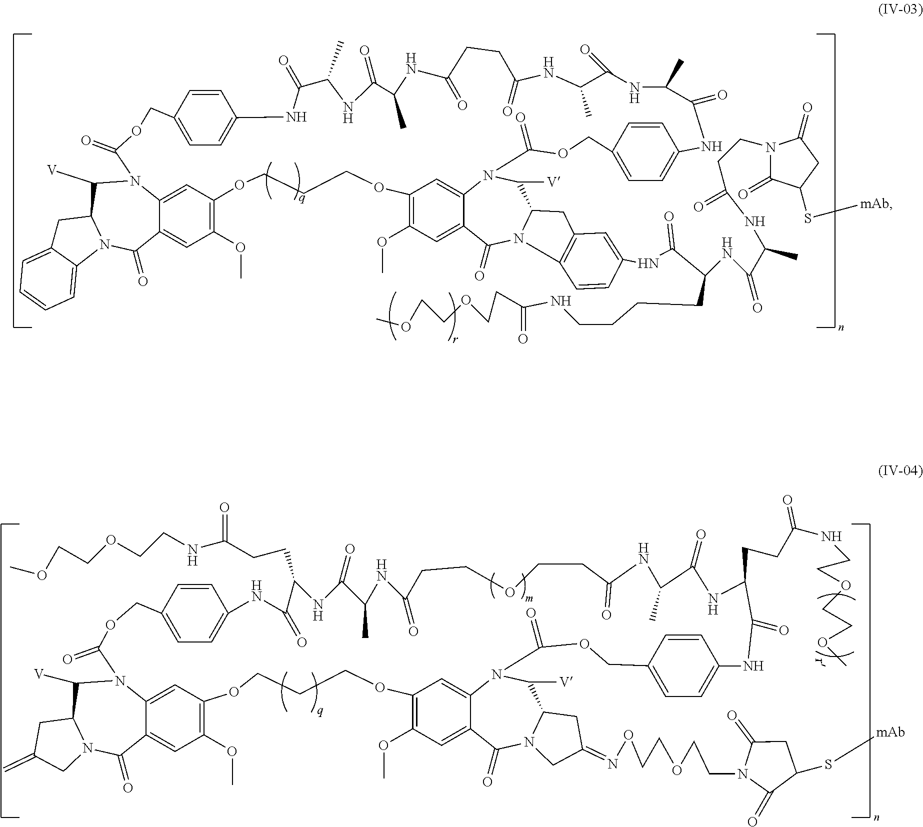

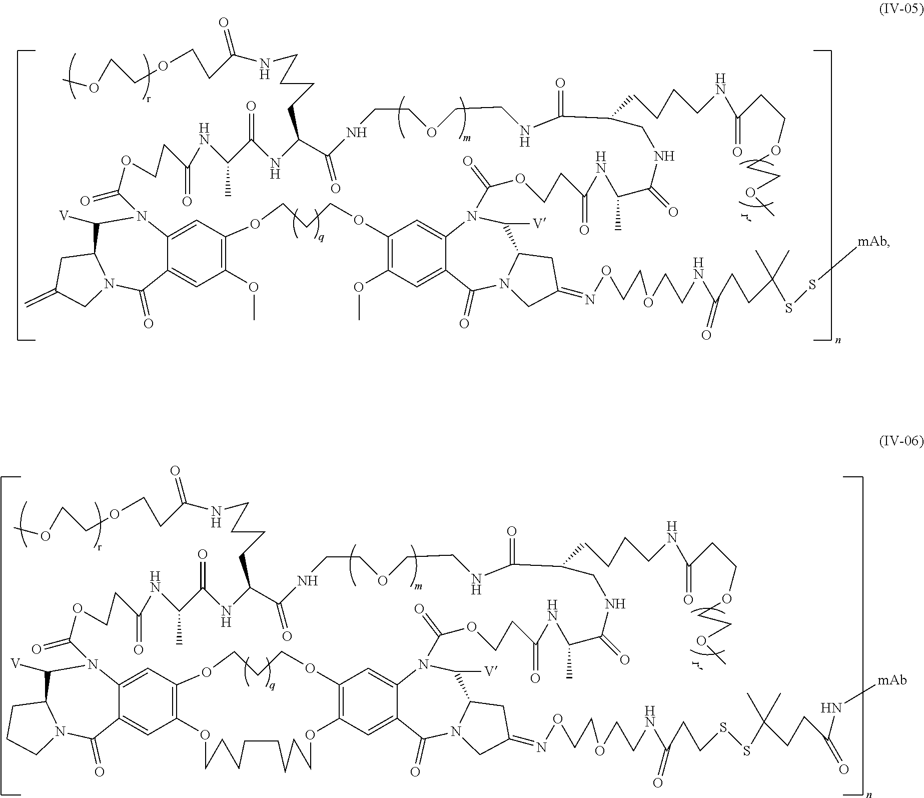

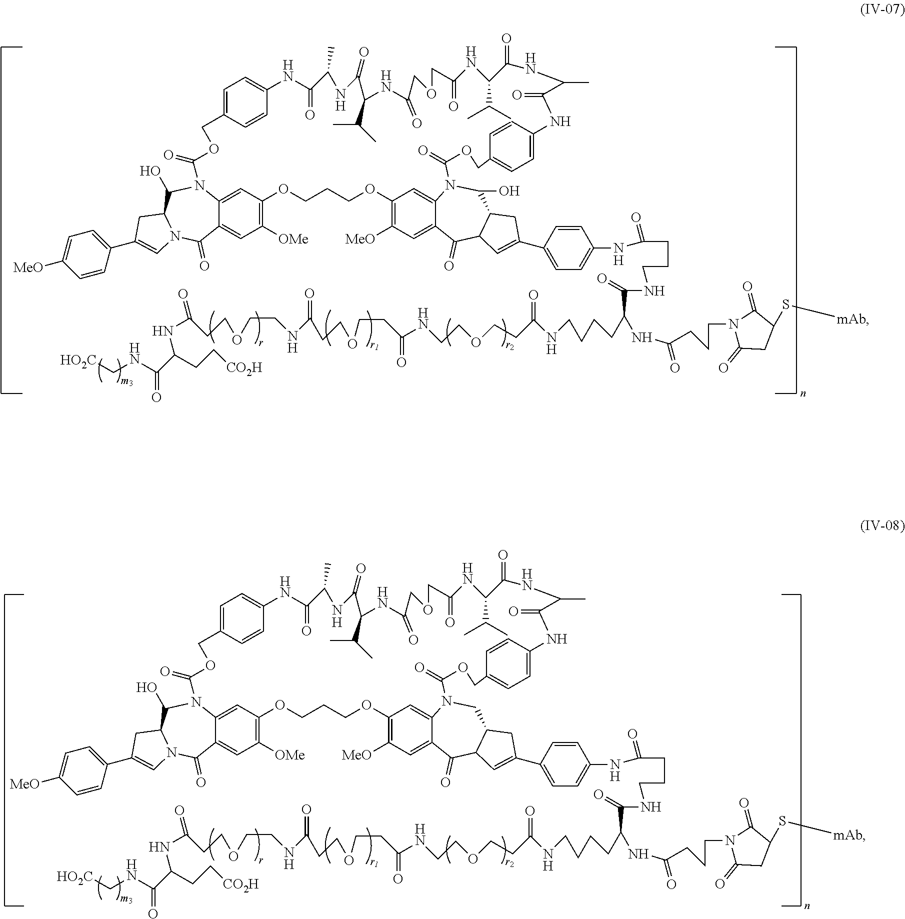

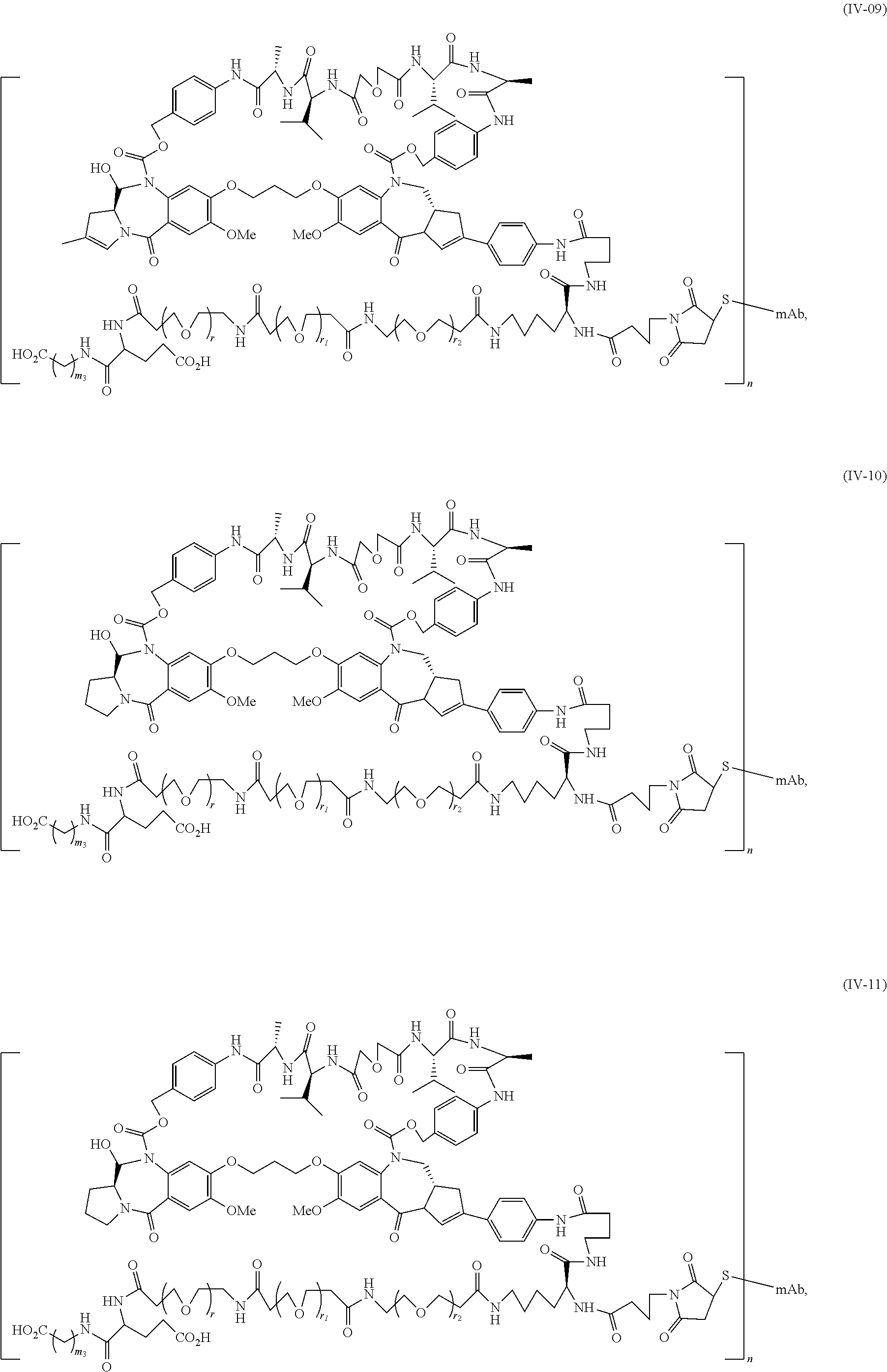

wherein [symbol] , -----, m, m′, n, q, and q′ are the same defined as in Formula (I); r, r′, and r″ are independently 0-200, m₃ is 0-30.

In another preferred embodiment, the conjugates of Formula (I), (II) (III), (IV) and (V) are prepared from coupling of a cell-binding molecule with a PBD dimer derivative having the Formula (V) (VI), (VII), and (VIII) accordingly as shown below

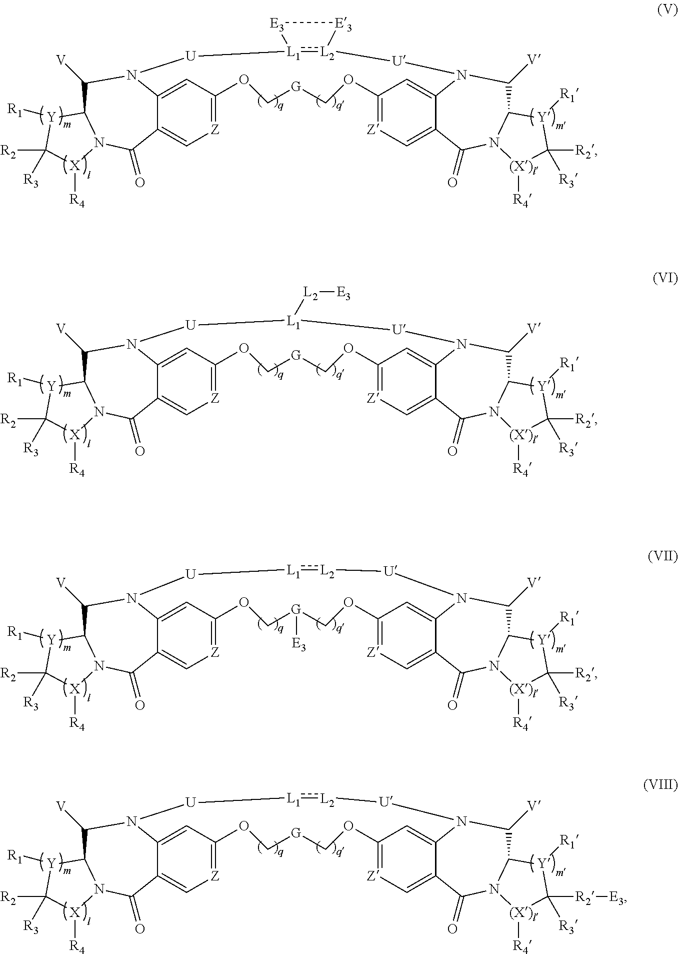

wherein [symbol] , -----, X, X′, Y, Y′, Z, Z′, l, l′, m, m′, n, q, q′, R₁, R₁′, R₂, R₂′, R₃, R₃′, R₄, R₄′, V, V′, U, U′ L₁, L₂, E₁, and E₂ are the same defined as in Formula (I);

wherein E₃ and E′₃ are independently selected from:

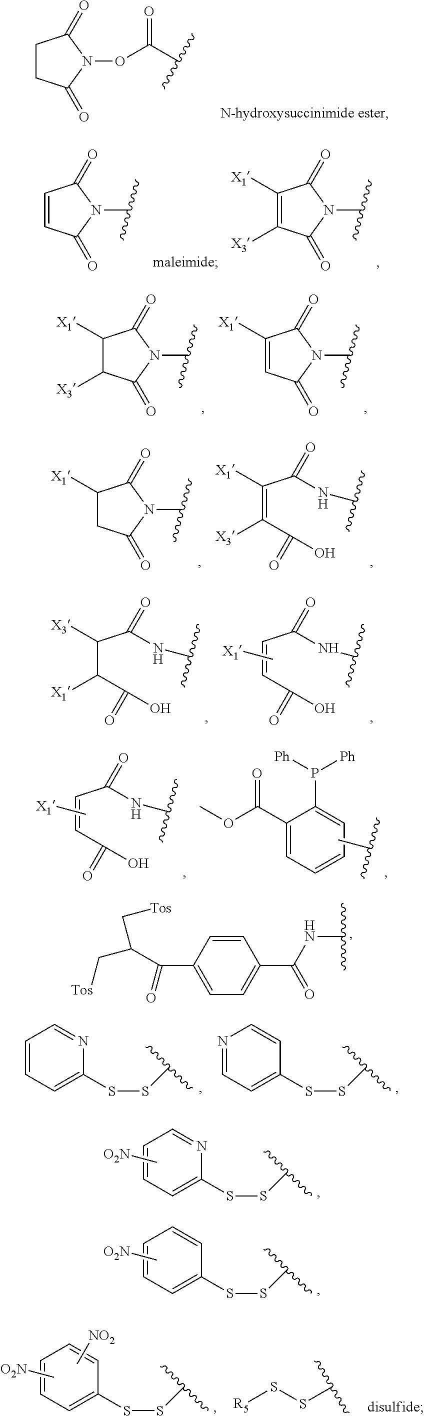

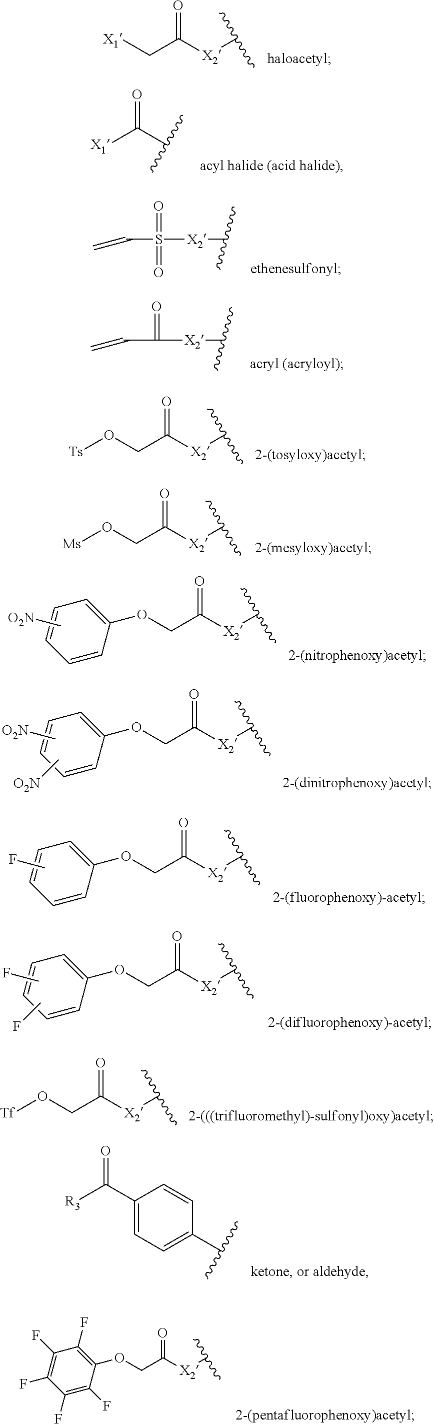

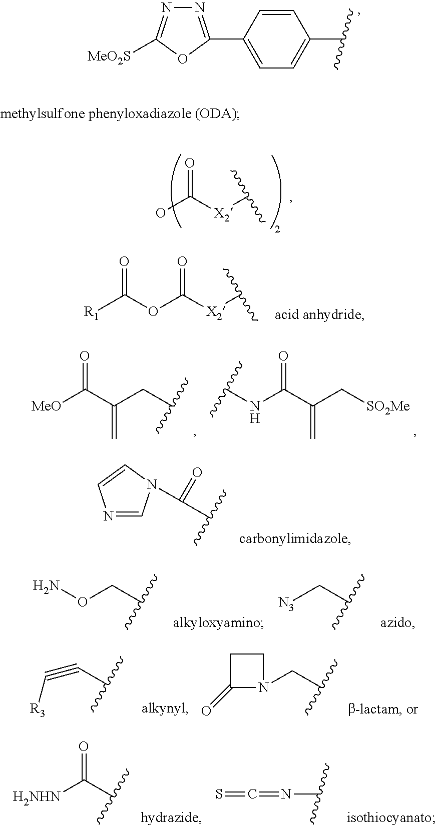

wherein X₁′ and X₃′ are independently F, Cl, Br, I or Lv₃; X₂′ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from methanesulfonyl (mesyl), toluenesulfonyl (tosyl), trifluoromethyl-sulfonyl (triflate), trifluoromethylsulfonate, nitrophenoxyl, phenylthio, pyridinylthio, N-succinimidyloxyl (NHS), phenoxyl; dinitrophenoxyl; pentafluorophenoxyl, tetrafluoro-phenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-yl, phenyloxadiazol-yl (ODA), oxadiazol-yl, or an intermediate molecule generated with a condensation reagent for Mitsunobu reactions, wherein R₁ and R₂ are defined above;

More preferably, E₃ and E′₃ are independently selected from —SH, —S—SCH₃, —S—SAc, —S—S-Pyridine, —S—S—Ar(—NO₂), —S-cell binding agent, or any one of the following formulas:

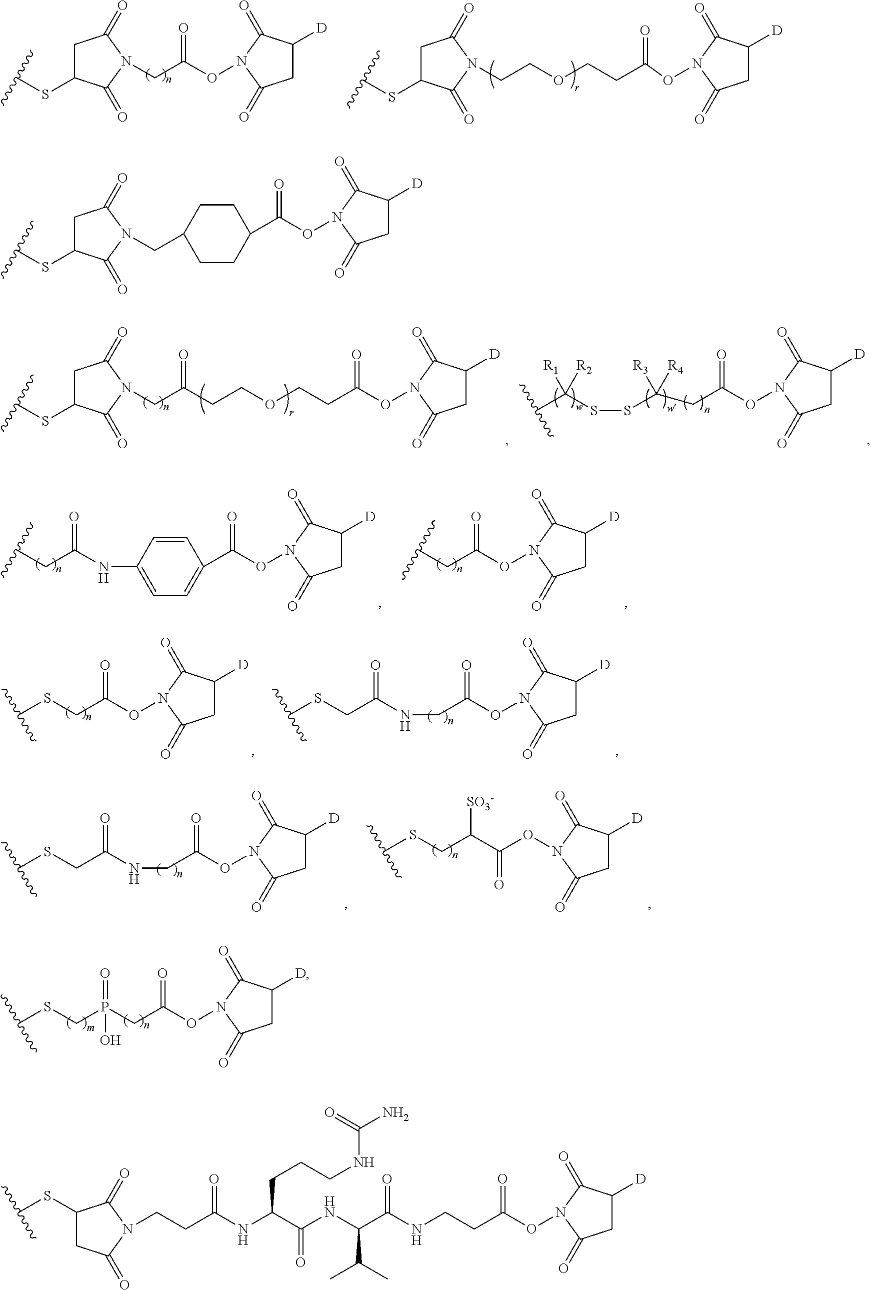

wherein D is H, —NO₂, SO³H, or F; wherein R₁, R₂, R₃, R₄, r, m and n are defined above; w and w′ are independently 0, 1 or 2;

wherein R₅ and R₅′ are independently selected from C₁˜C₆ alkyl, aryl, cyclic, cyclohetero, H, or M (wherein M is Na, K, Ca, ammonium or the other pharmaceutically acceptable salt);

In certain embodiments, the PBD derivatives of Formula (V), (VI), (VII), and (VIII) are represented by the following Formulas (V-01)˜(V-20), (VI-01)˜(VI-05), (VII-01)˜(VII-06), (VIII-01)˜(VIII-06) accordingly below:

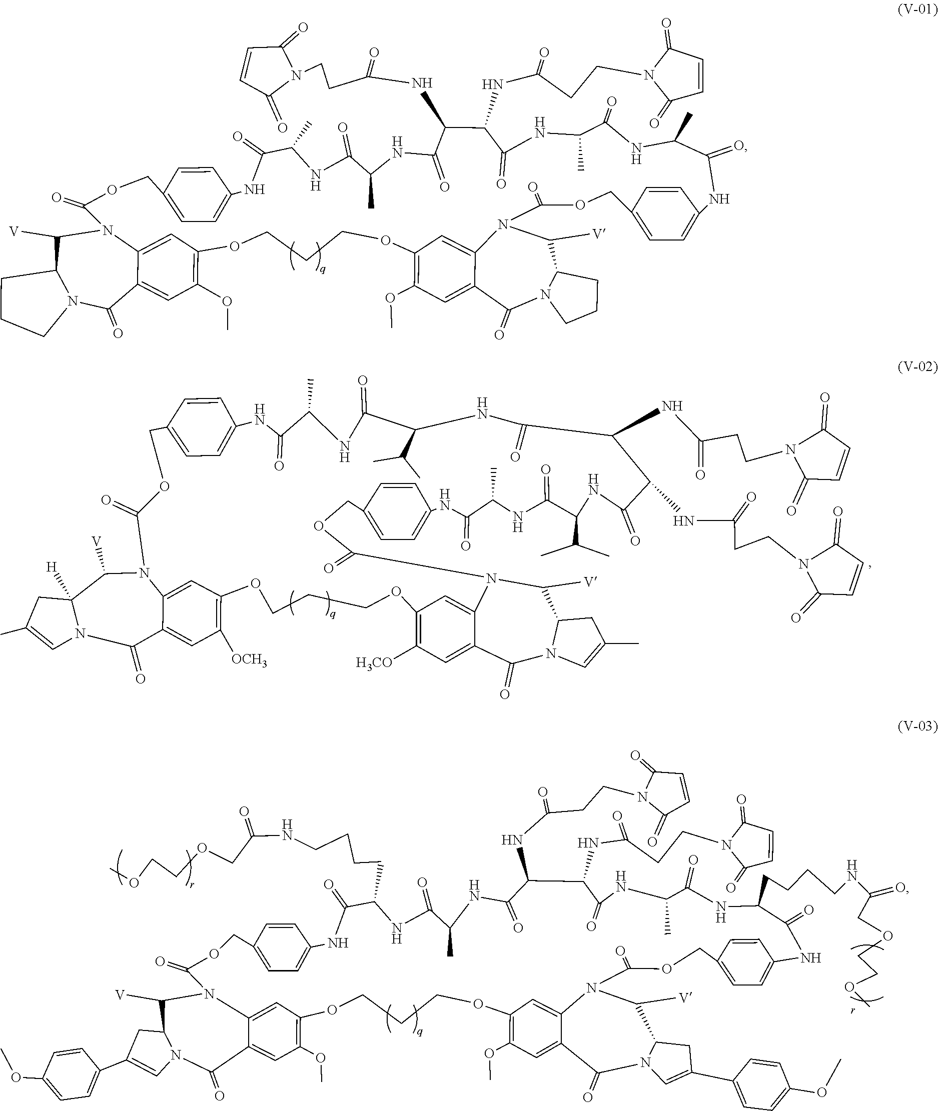

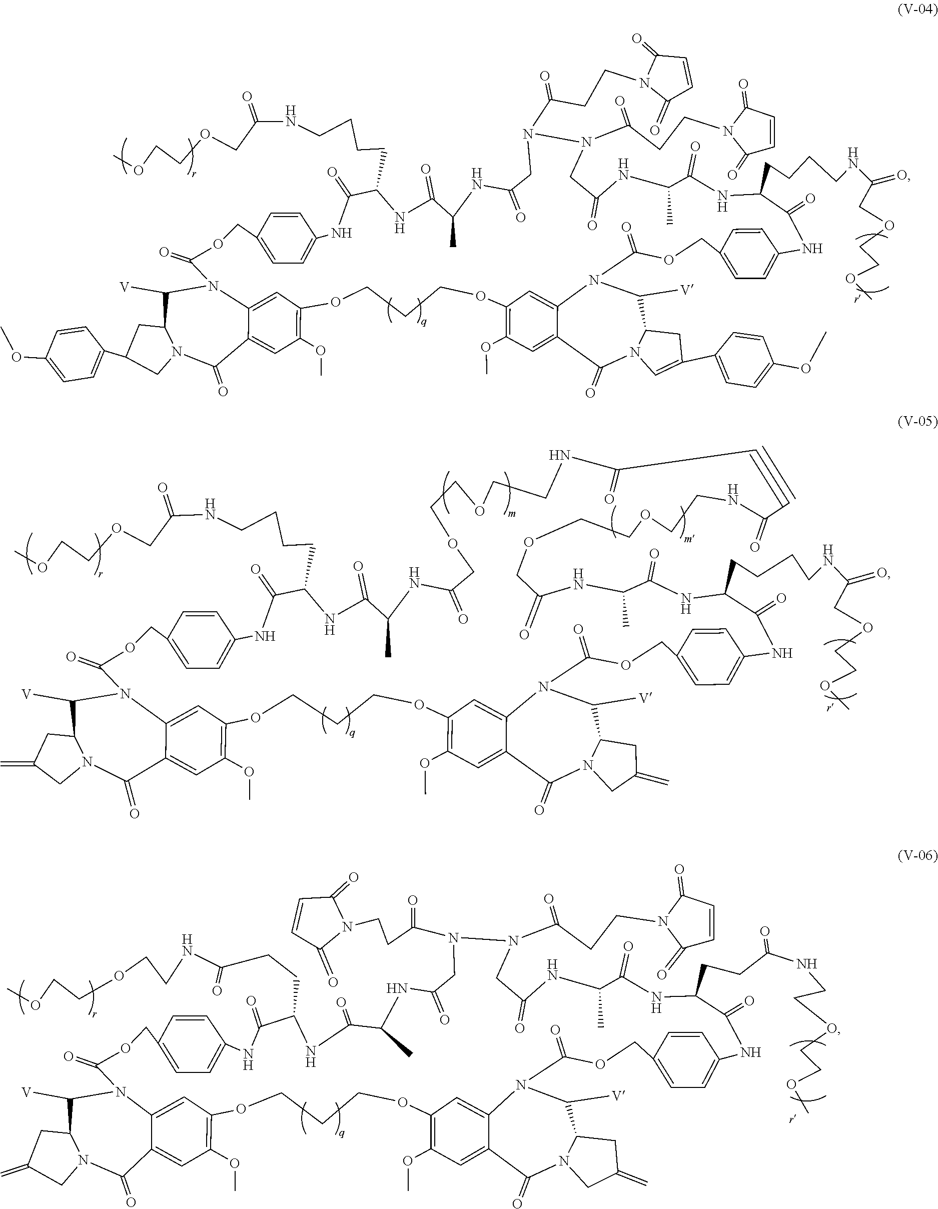

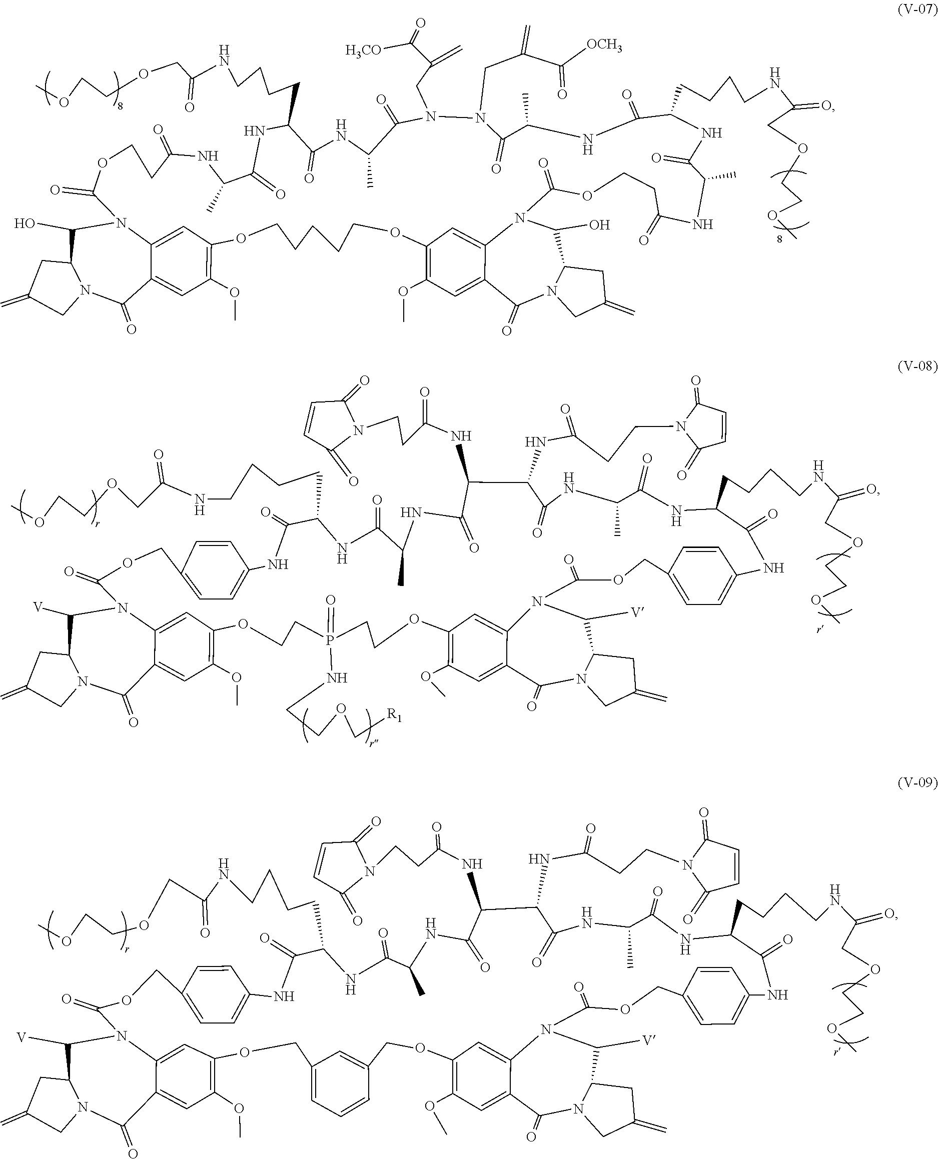

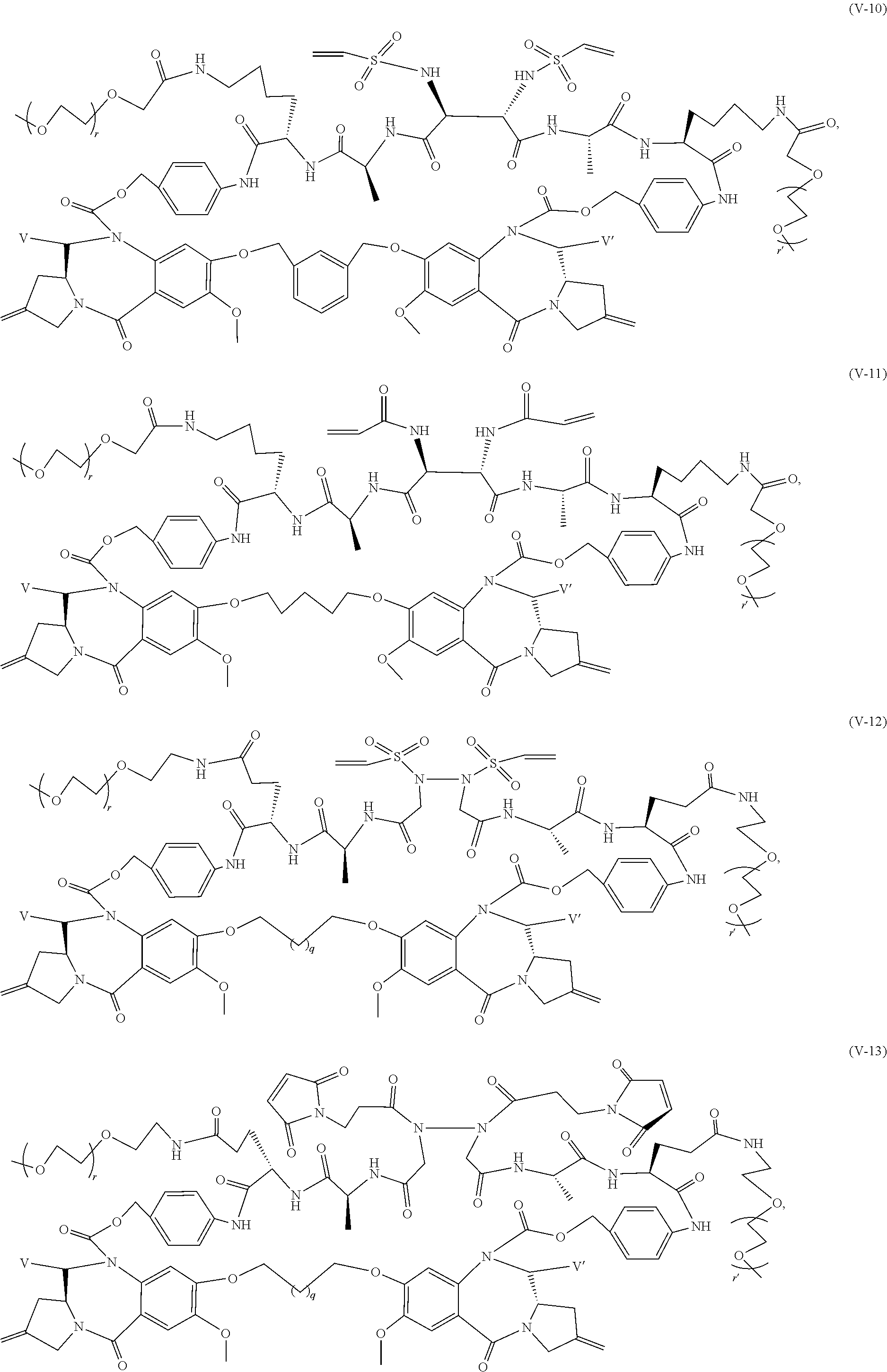

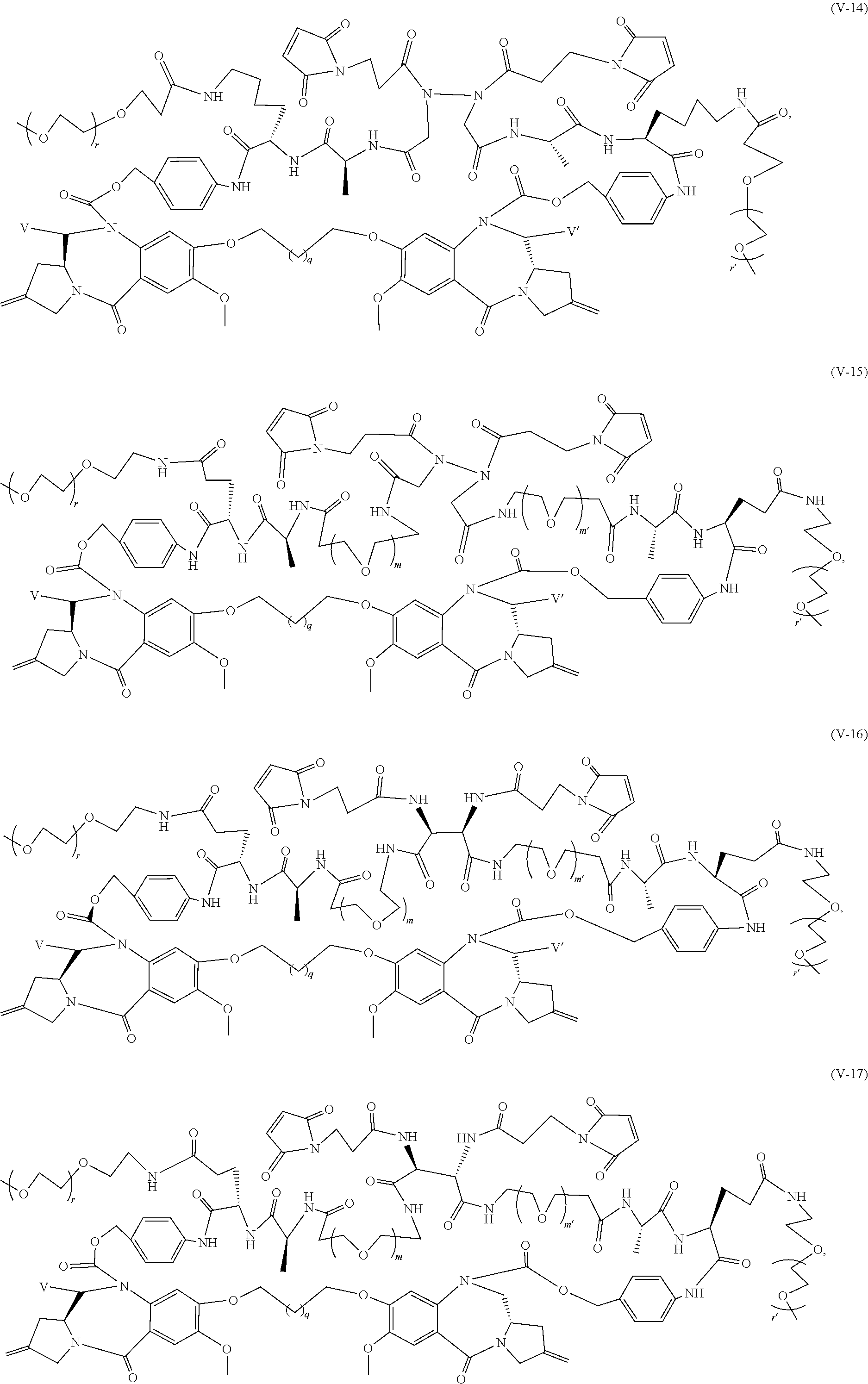

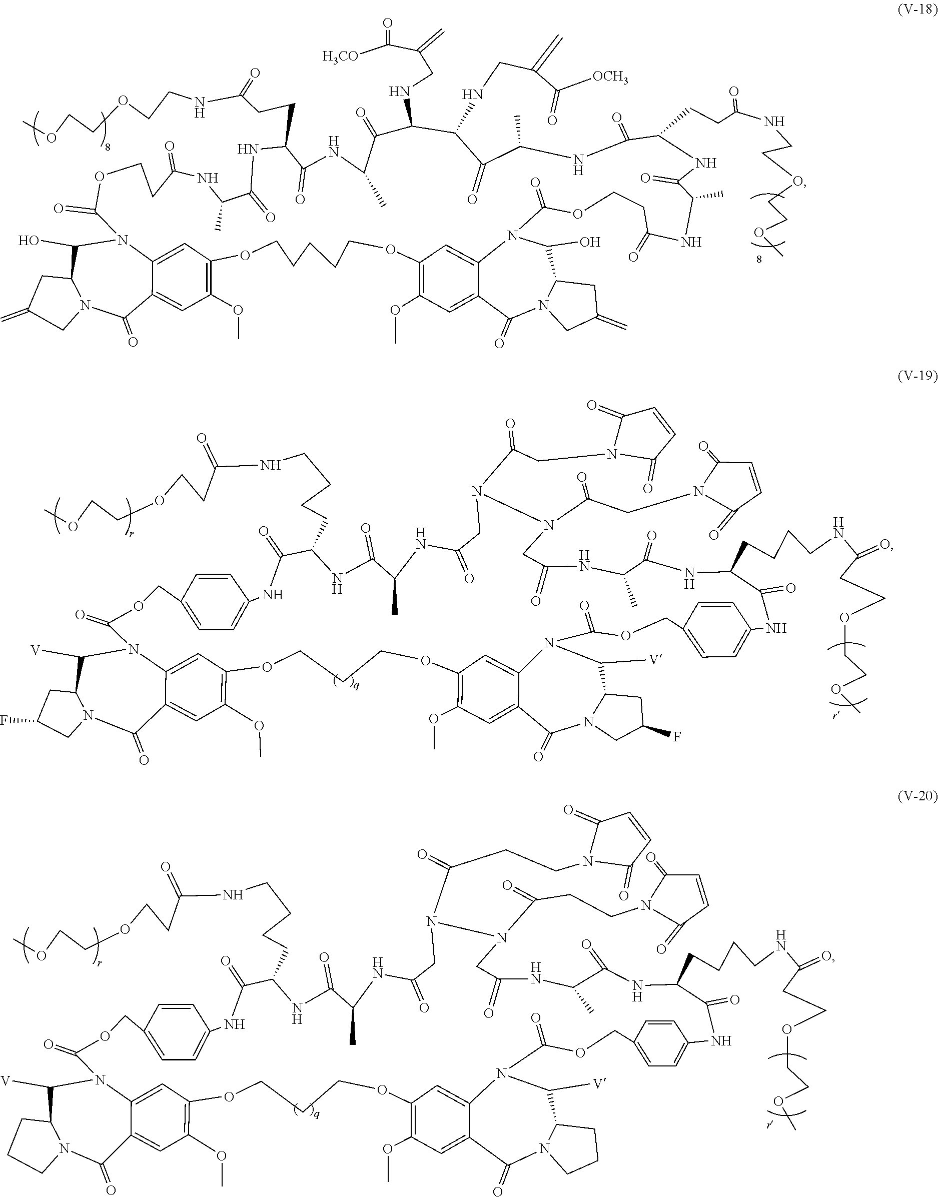

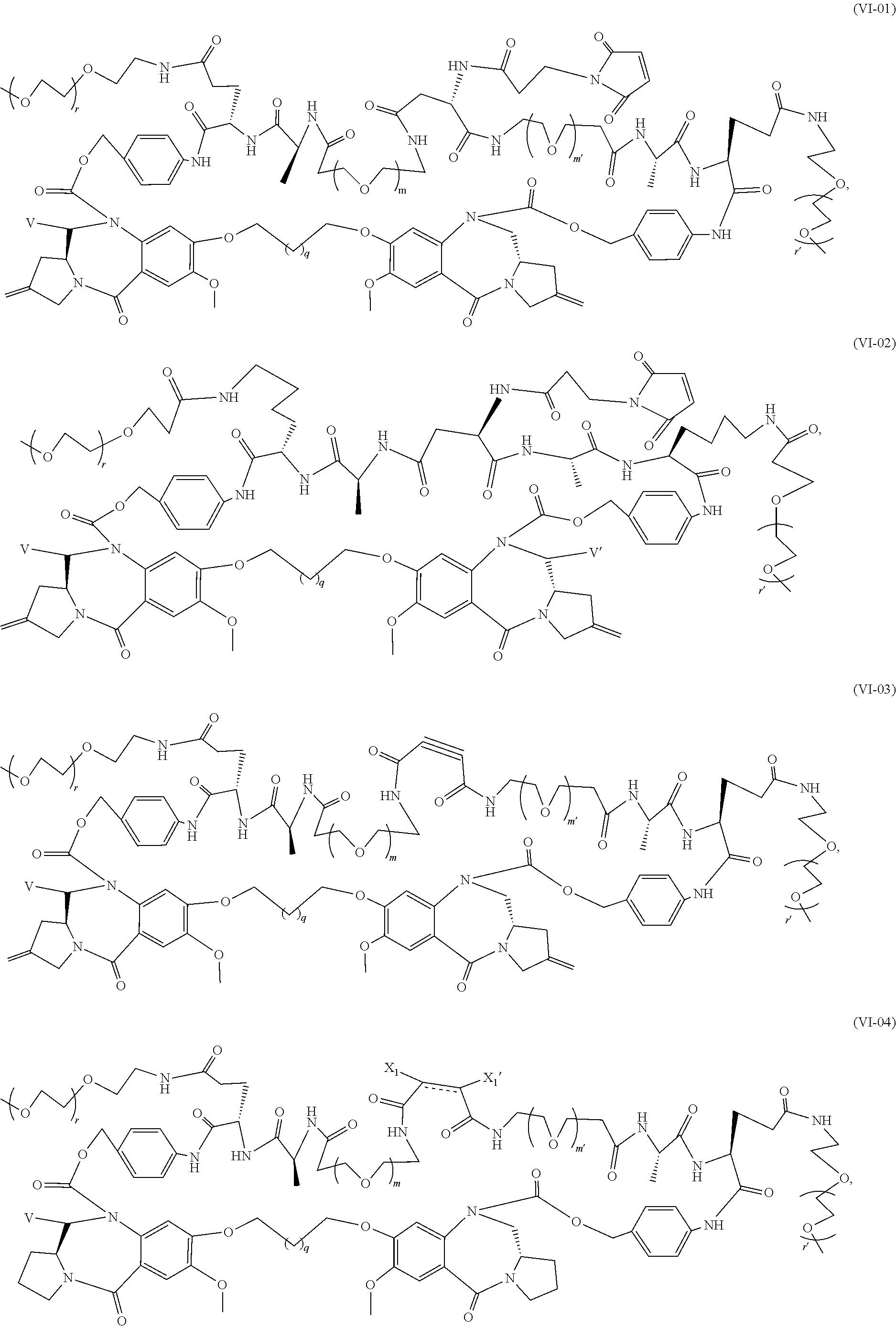

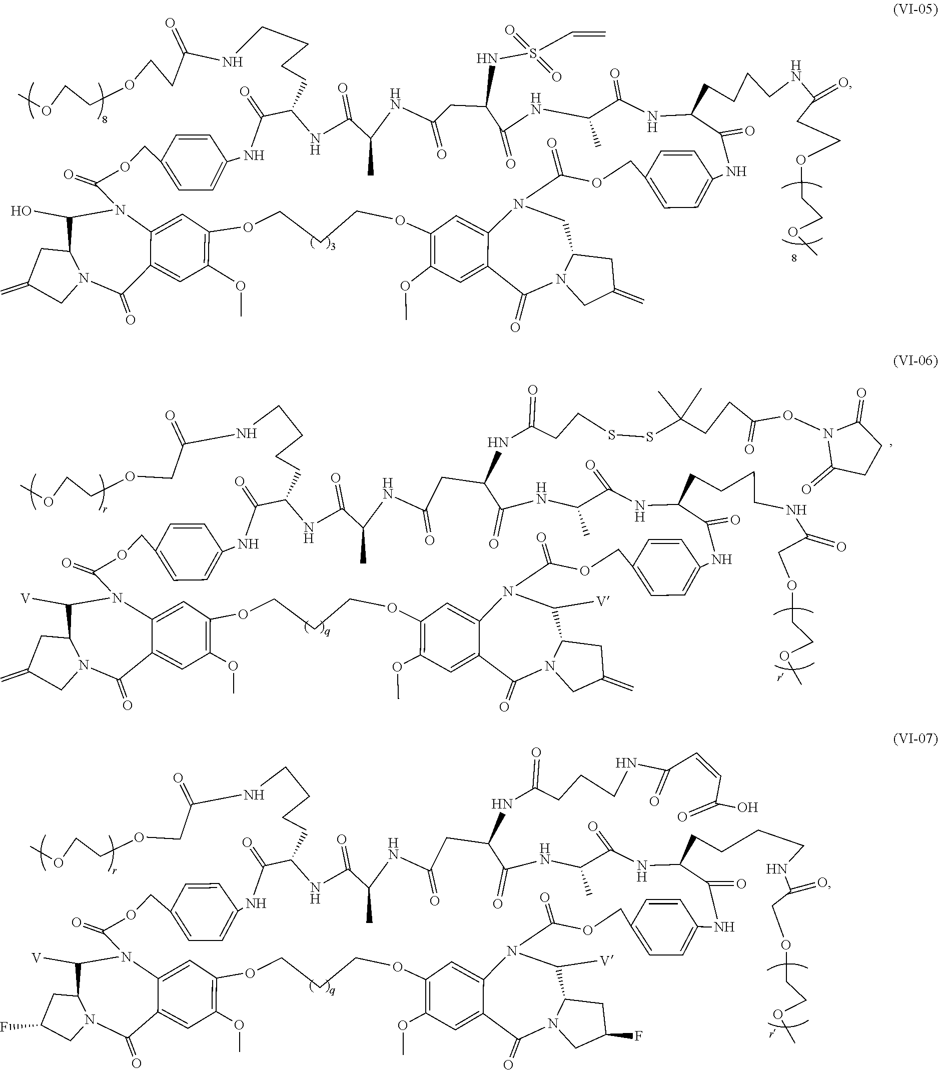

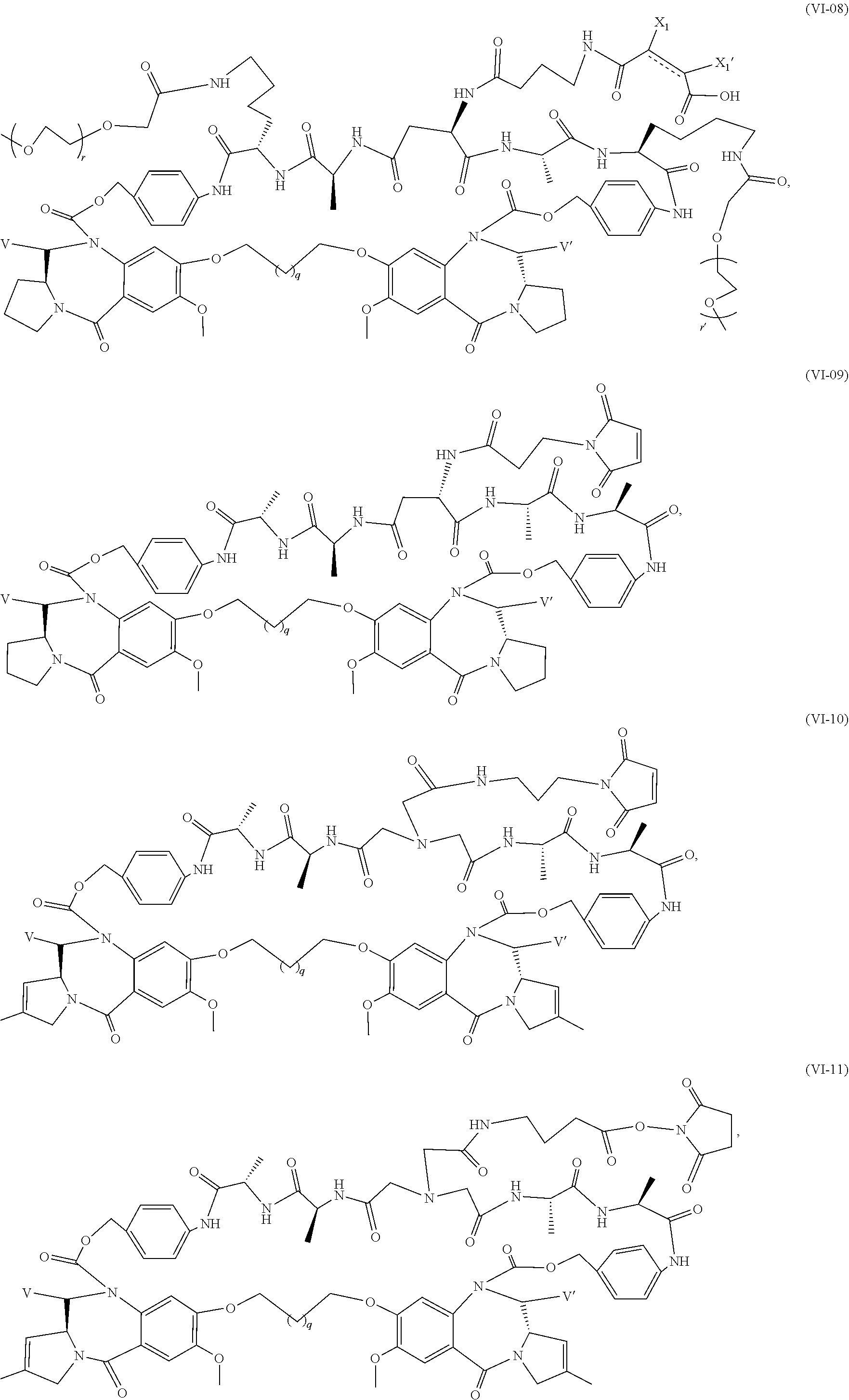

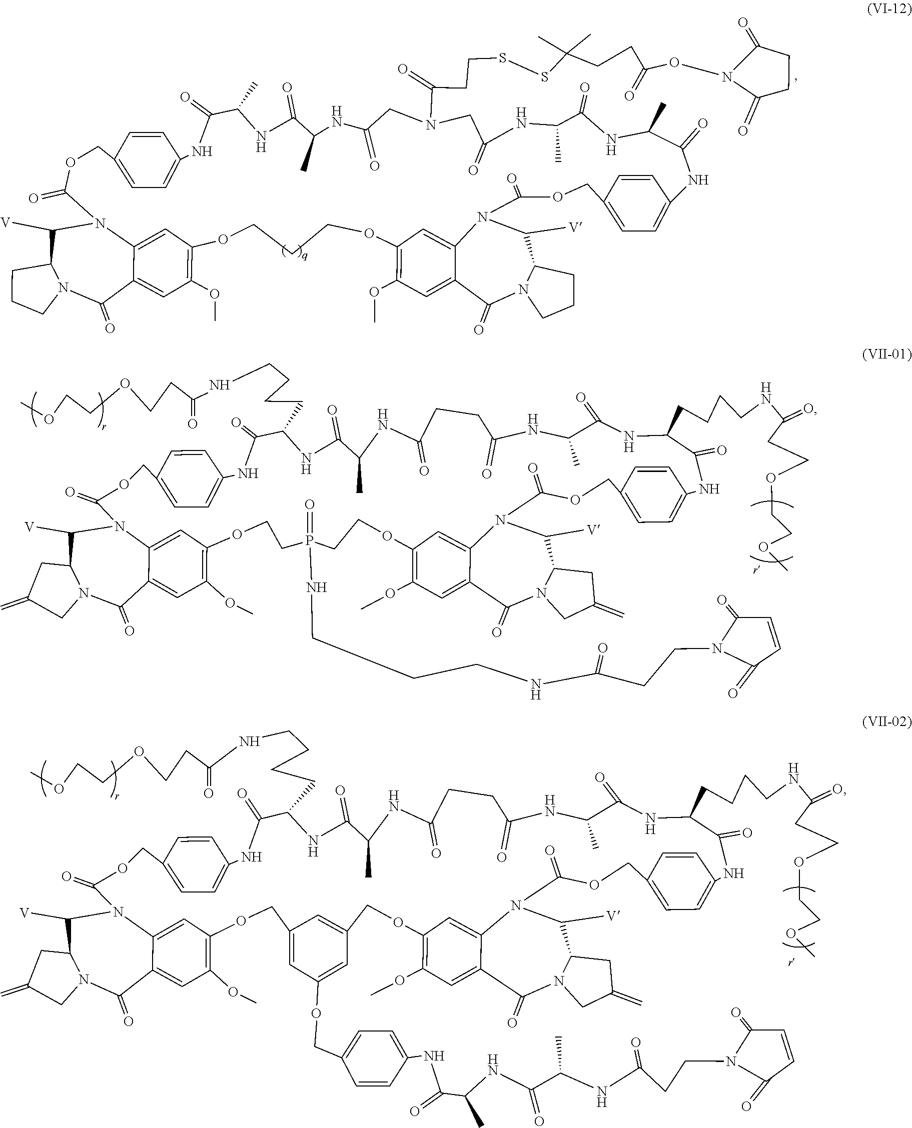

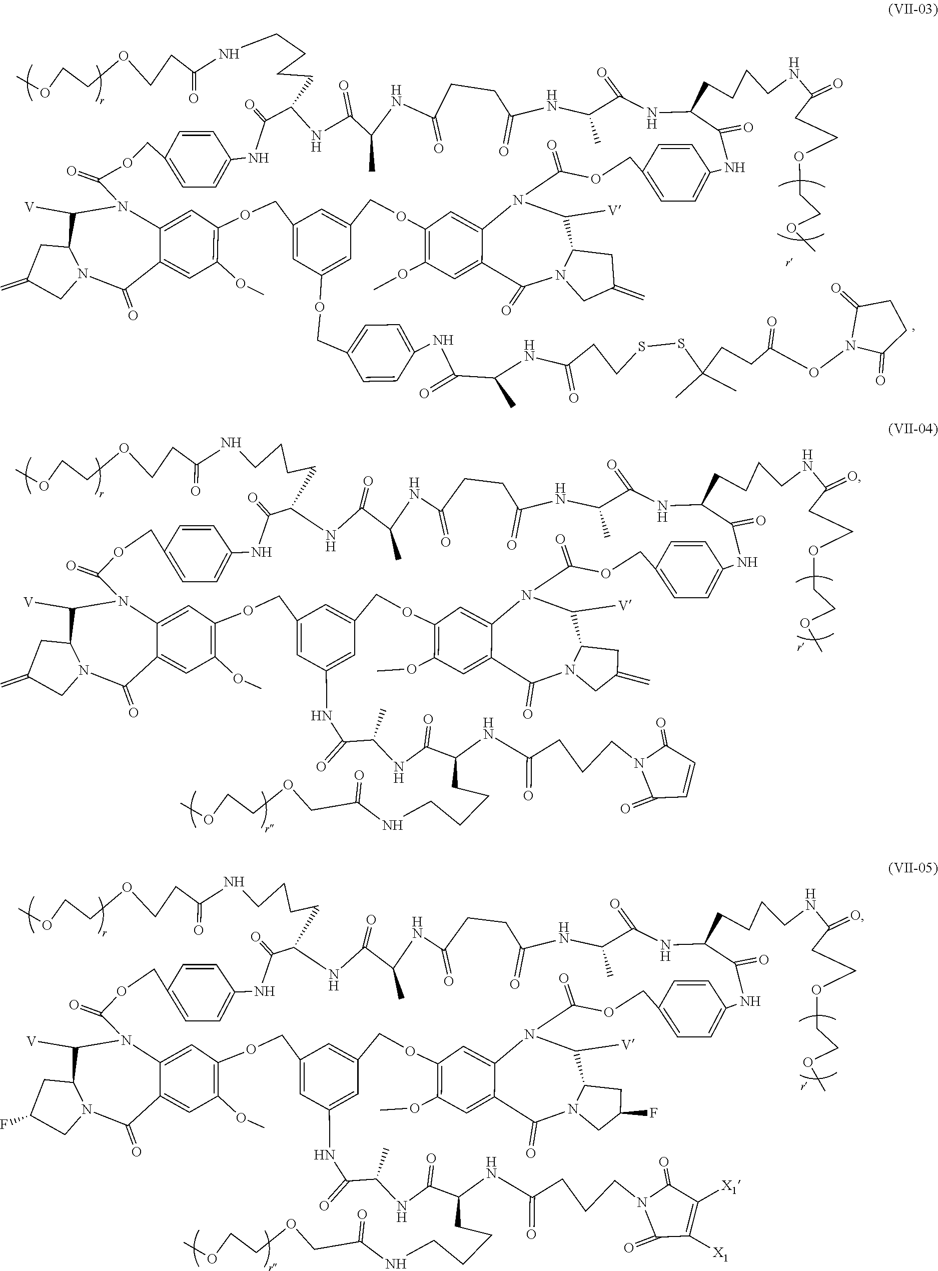

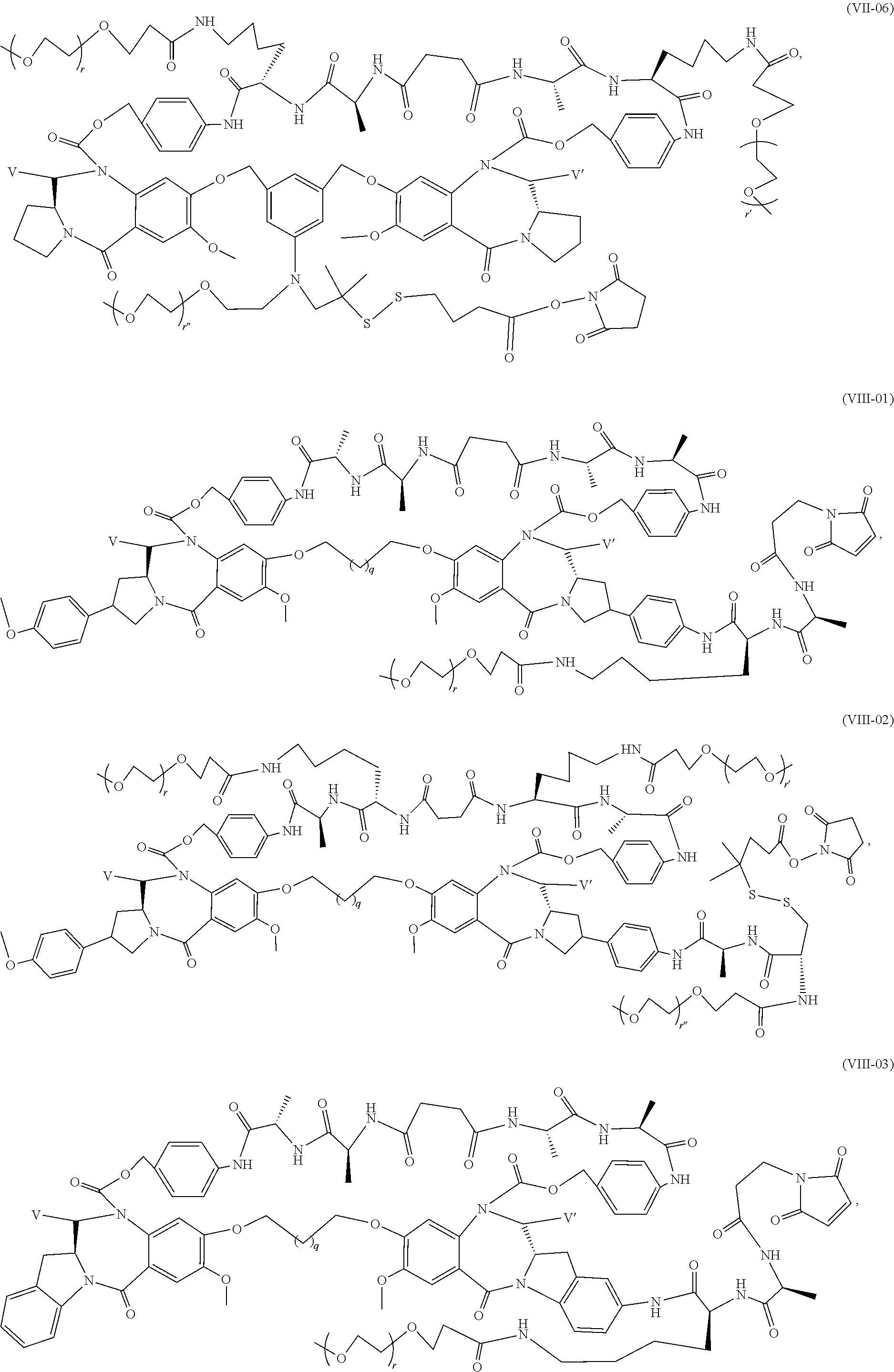

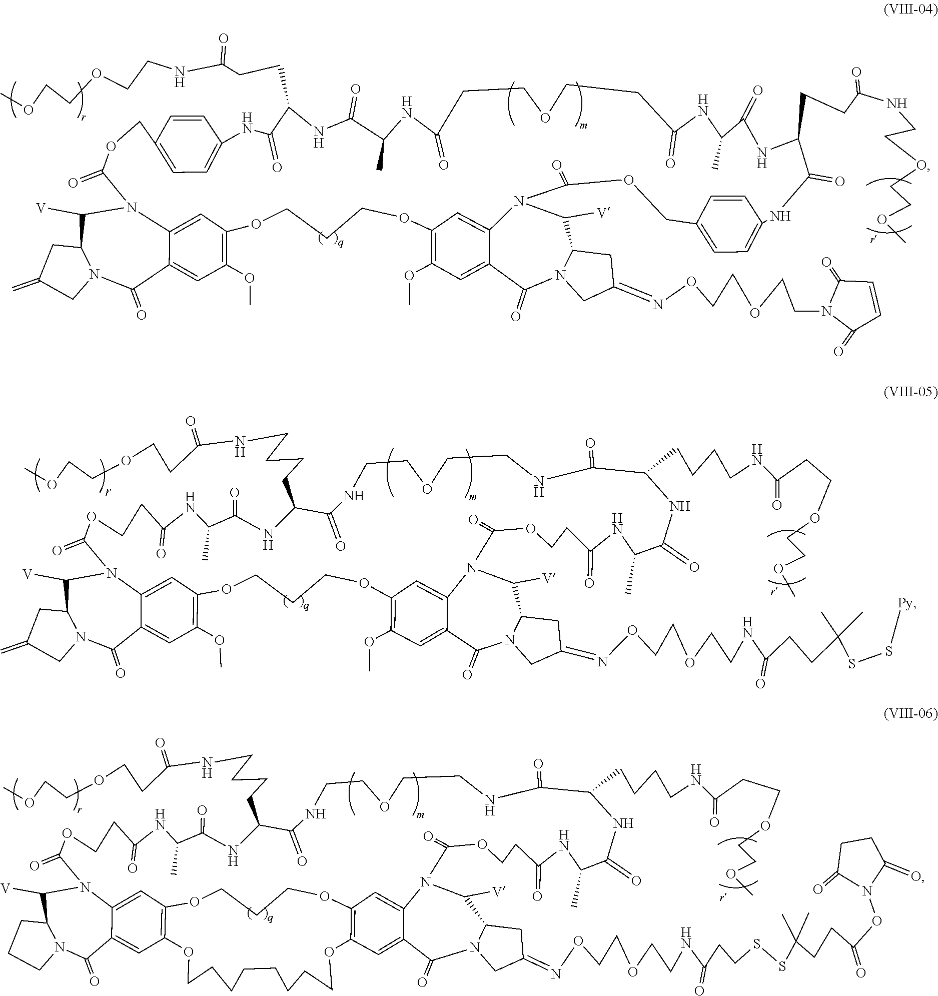

wherein U, U′, V, V′, n, n′, and L are defined the same as in Claim; R₆ and R₆′ are independently selected from C₁˜C₆ alkyl, aryl, cyclic, cyclohetero, halogen, haloalkyl, alkoxy, haloalkoxy alkylamino,—N₂, —CN or H; X₁ and X₁′ are independently H, F, Cl, Br, I, OTs (tosylate), OMs(mesylate), nitrophenol OAr(N₂), OAr(N₂)₂ dinitrophenol, OAr(F) monofluorophenol, OAr(F)₅ pentafluorophenol, OAr(F)₂ difluorophenol, N-hydroxysuccinimide (NHS), phenol, tetrafluorophenol, pentachlorophenol, triflate, imidazole, dichlorophenol, tetrachlorophenol, 1-hydroxybenzotriazole, or 2-ethyl-5-phenylisoxazolium-3′-sulfonate.

Synthesis of the Cross-Linked PBD Dimer Derivatives of Formula (V), (VI), (VII) and (VIII) as Cytotoxic Agents.

The compounds and process of the present invention can be prepared in a number of ways well known to those skilled in the art. The compounds can be synthesized, for example, by application or adaptation of the methods described in the examples, or variations thereon as appreciated by the skilled artisan. The appropriate modifications and substitutions will be readily apparent and well known or readily obtainable from the scientific literature to those skilled in the art. In particular, such methods can be found in Richard C. Larock, Comprehensive Organic Transformations, A Guide to Functional Group Preparations, Two Volume Set, 2nd Edition, Wiley Publishers, 2010.

Because the cytotoxic agents of the present invention may contain one or more asymmetrically substituted carbon atoms, and may be isolated in optically active or racemic forms, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

The cytotoxic agents of the present invention may be prepared by a variety of synthetic routes. The reagents and starting materials are commercially available, or readily synthesized by well-known techniques by one of ordinary skill in the arts. All substituents, unless otherwise indicated, are as previously defined.

In the synthetic reactions of the cytotoxic agents of the present invention, it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see Peter G. M. Wuts, Theodora W. Greene in Greene's Protective Groups in Organic Synthesis, 4th Edition, John Wiley and Sons, 2006; Ian T. Harrison, Shuyen Harrison in Compendium of Organic Synthetic Methods, Vol 1,2 Vols. 1& 2 By Ian T. Harrison & Shuyen Harrison, Vols 3˜5 by Louis S. Hegedus, Leroy Wade Vols 6-Vol 12 by Michael B. Smith, John Wiley and Sons, 2006˜2012.

Normally the synthetic reactions are carried out in suitable solvents, temperatures and time. A variety of solvents which have no adverse effect on the reaction or on the reagents involved can be used in a synthetic reaction of the cytotoxic agent. Examples of suitable solvents include: hydrocarbons, which may be aromatic, aliphatic or cycloaliphatic hydrocarbons, such as hexane, cyclohexane, benzene, toluene and xylene; hydrocarbons containing halogens, such as chloroform, dichloromethane, dichloroethane; amides, such as dimethylactamide or dimethylformamide; alcohols such as ethanol and methanol and ethers, such as diethyl ether and tetrahydrofuran. The reactions can take place over a wide range of temperatures, from −100° C.˜300° C. More preferably from 0° C. to 150° C. The time required for the synthetic reaction may also vary widely, depending on many factors, notably the reaction temperature and the nature of the reagents and can be from 5 second to 4 weeks, more preferably from 10 min to 24 hours. In addition, the cytotoxic agents prepared may be isolated or purified from the reaction mixture by conventional means, such as evaporating or distilling off the solvent from the reaction mixture, or after distilling off the solvent from the reaction mixture, pouring the residue into water followed by extraction with a water-immiscible organic solvent and then distilling off the solvent from the extract. It may also involve various well known techniques, such as re-crystallization, re-precipitation or the various chromatography techniques, notably column chromatography, preparative thin layer chromatography, or high performance liquid chromatography.

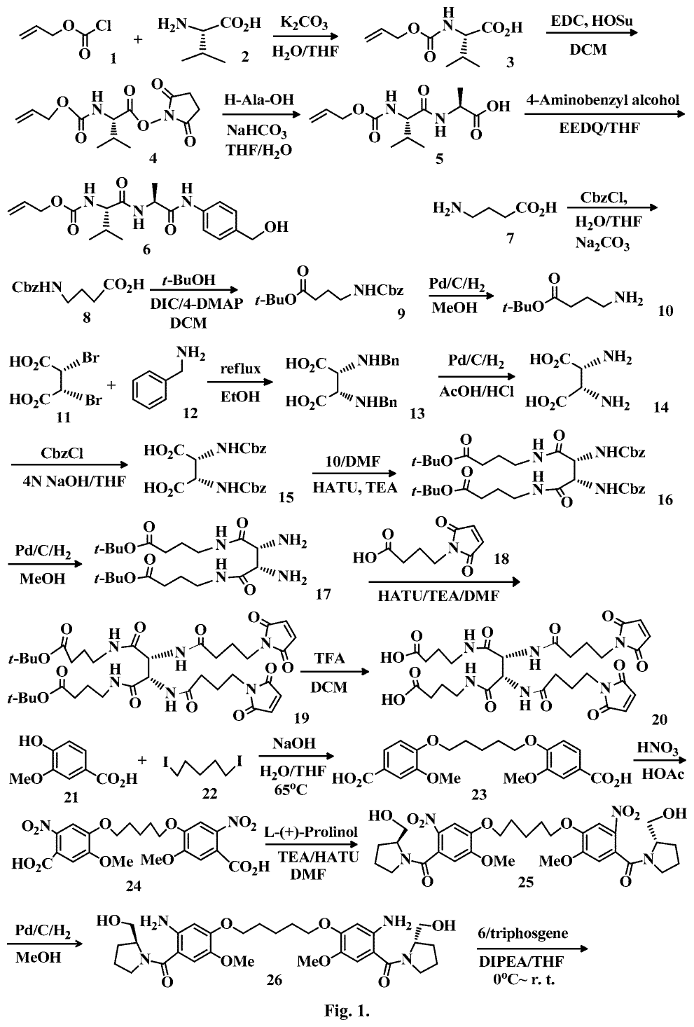

Some of the synthetic reactions of the cytotoxic agents and their conjugates to a cell binding agent are further exampled but not restricted in the FIGS. 1˜32 and in the examples of the following description.

The Conjugates of Cross-Linked PBD Dimers to a Cell-Binding Molecule.

The present invention provides a conjugate molecule comprising at least one PBD derivative covalently linked to a cell-binding agent (Q) through a linking group of a conjugate linker. Preferably said conjugate comprises one to twenty molecules of cross-linked PBD dimer derivatives according to the invention covalently linked to a cell-binding agent through a linking group of a linker on a cross-linked PBD dimer derivative.

As stated above, the conjugates of a cell surface binding molecule-cross-linked PBD dimer derivatives are illustrated in the Formula (I), (II), (III) and (IV) above.

Drug loading may range from 1 to 30 drug moieties (D) per antibody and is preferred the average number of 2˜8 drug moieties per antibody in a molecule of Formula (I)˜(IV). The average number of drug moieties per antibody in preparations of ADC from conjugation reactions may be characterized by conventional means such as mass spectroscopy, ELISA assay, and HPLC. The quantitative distribution of the conjugates in terms of the drug loading may also be determined. In some instances, separation, purification, and characterization of homogeneous the conjugates where Drug loading is a certain value from the conjugates with the drug loadings may be achieved by means such as reverse phase HPLC or electrophoresis.

The Cell binding agents (CBA) may be of any kind and include peptides and non-peptides. Generally, the cell binding agents include, but are not limited to, large molecular weight proteins such as, for example, full-length antibodies (polyconal and monoclonal antibodies); single chain antibodies; fragments of antibodies such as Fab, Fab′, F(ab′)₂, Fᵥ, [Parham, J. Immunol. 131, 2895-2902 (1983)], fragments produced by a Fab expression library, anti-idiotypic (anti-Id) antibodies, CDR's, and epitope-binding fragments of any of the above which immunospecifically bind to cancer cell antigens, viral antigens or microbial antigens; antibody mimetic, such as an affibody; domain antibodies (dAb); nanobodies; unibodies; DARPins; anticalins; versabodies; duocalins; lipocalins; vimers; interferons (such as type I, II, III); peptides; lymphokines such as IL-2, IL-3, IL-4, IL-6, IL-8, IL-10, IL-12, IL-13, GM-CSF, interferon-gamma (IFN-γ); hormones such as insulin, TRH (thyrotropin releasing hormones), MSH (melanocyte-stimulating hormone), steroid hormones, such as androgens and estrogens, melanocyte-stimulating hormone (MSH); growth factors and colony-stimulating factors such as epidermal growth factors (EGF), granulocyte-macrophage colony-stimulating factor (GM-CSF), transforming growth factors (TGF), such as TGFα, TGFβ, insulin and insulin like growth factors (IGF-I, IGF-II) G-CSF, M-CSF and GM-CSF [Burgess, Immunology Today, 5, 155-158 (1984)]; vaccinia growth factors (VGF); fibroblast growth factors (FGFs); smaller molecular weight proteins, poly-peptide, peptides and peptide hormones, such as bombesin, gastrin, gastrin-releasing peptide; platelet-derived growth factors; interleukin and cytokines, such as interleukin-2 (IL-2), interleukin-6 (IL-6), leukemia inhibitory factors, granulocyte-macrophage colony-stimulating factor (GM-CSF); vitamins, such as folate; apoproteins and glycoproteins, such as transferrin {O'Keefe et al, 260 J. Biol. Chem. 932-937 (1985)}; sugar-binding proteins or lipoproteins, such as lectins; cell nutrient-transport molecules; and small molecular inhibitors, such as prostate-specific membrane antigen (PSMA) inhibitors and small molecular tyrosine kinase inhibitors (TKI), non-peptides or any other cell binding molecule or substance, such as bioactive polymers (Dhar, et al, Proc. Natl. Acad. Sci. 2008, 105, 17356-61); dendrimers (Lee, et al, Nat. Biotechnol. 2005, 23, 1517-26; Almutairi, et al; Proc. Natl. Acad. Sci. 2009, 106, 685-90); nanoparticles (Liong, et al, ACS Nano, 2008, 19, 1309-12; Medarova, et al, Nat. Med. 2007, 13, 372-7; Javier, et al, Bioconjugate Chem. 2008, 19, 1309-12); liposomes (Medinai, et al, Curr. Phar. Des. 2004, 10, 2981-9); viral capsides (Flenniken, et al, Viruses Nanotechnol. 2009, 327, 71-93). In general monoclonal antibodies are preferred as a cell-surface binding agent if an appropriate one is available.

The linker used for the conjugation of this invention includes, but not limited to, a disulfide linker, a thioether linker, an amide bonded linker, a peptidase-labile linker, a photolabile linker, an acid-labile linkers (such as hydrazone liner), an esterase-labile linker, an oxidatively labile linker, a metabolically labile linker, a biochemically labile linker.

Preferably, the linker is linked to the cell binding agent via a function reactive towards for instance thiol and amino functions of the cell binding agent coming from reduced disulfide bonds and lysine residues respectively. More particularly, said derivative is linked through the —CO— group to the amino function of the lysine residue of said cell binding agent, so as to form an amide bond.

In addition, the linker may be composed of one or more linker components. Exemplary linker components include 6-maleimidocaproyl (“MC”), maleimidopropanoyl (“MP”), valine-citrulline (“val-cit” or “vc”), alanine-phenylalanine (“ala-phe” or “af”), glycine-glycine, p-aminobenzyloxycarbonyl (“PAB”), N-succinimidyl 4-(2-pyridylthio)pentanoate (“SPP”), N-succinimidyl 4-(N-maleimidomethyl)cyclohexane-1 carboxylate (“SMCC”), N-Succinimidyl (4-iodo-acetyl)aminobenzoate (“SIAB”), ethyleneoxy (—CH₂CH₂O—) as one or more repeating units (“EO” or “PEO”). The linker may be a “cleavable linker,” facilitating release of a drug in the cell. Additional linker components are known in the art and some are illustrated below:

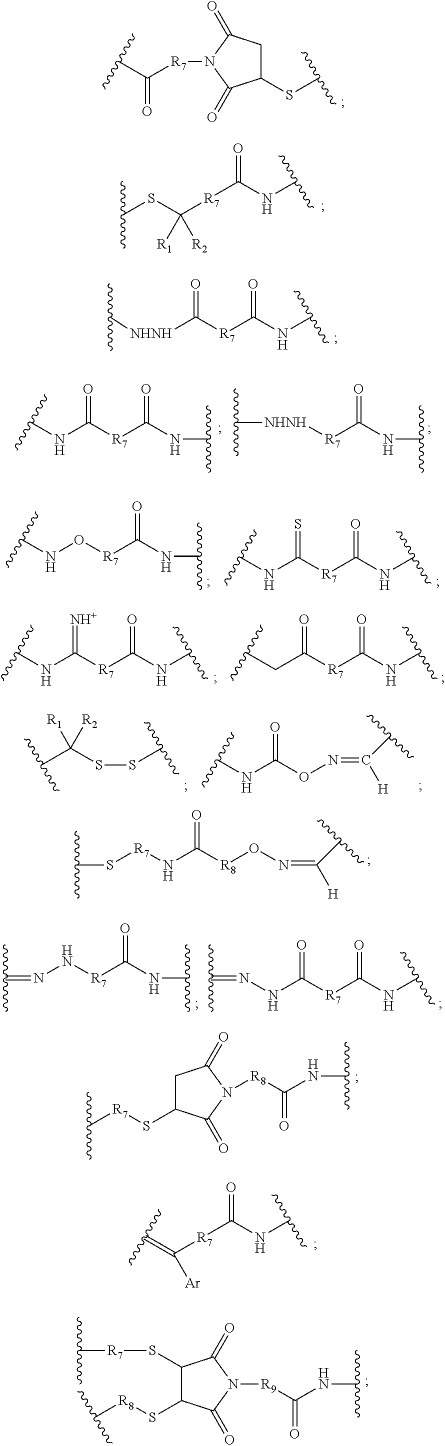

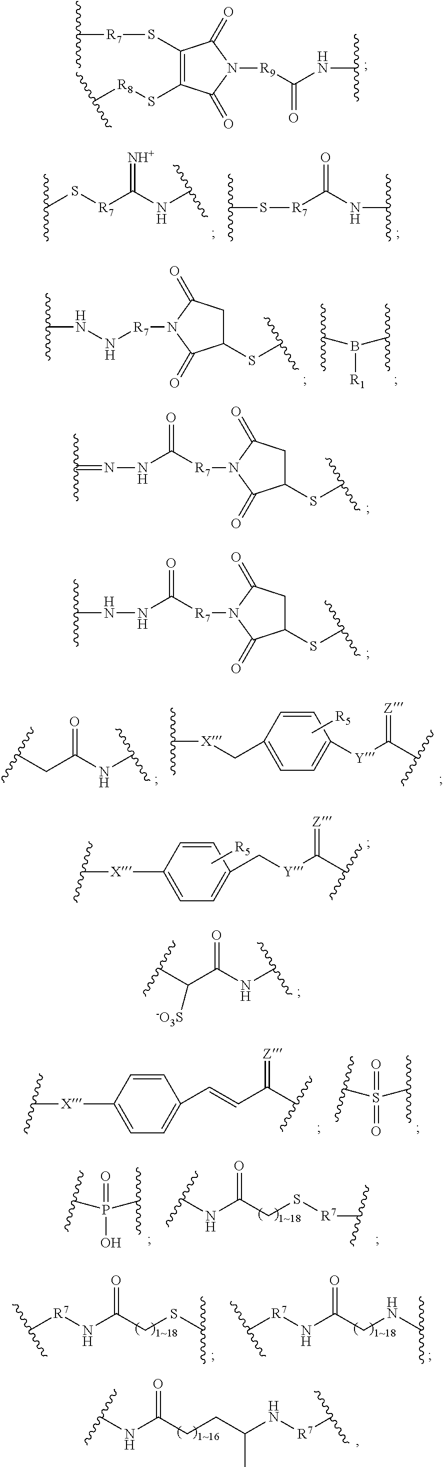

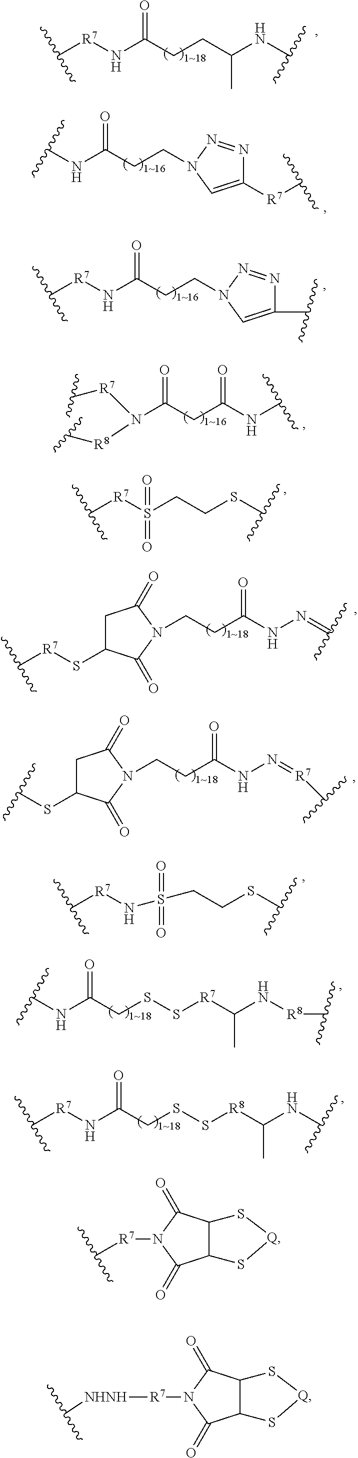

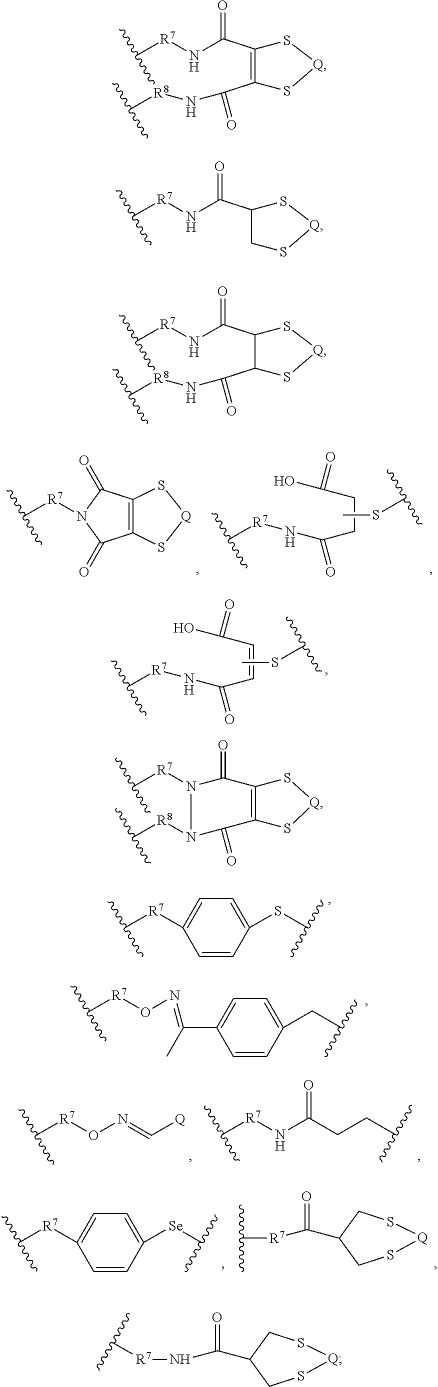

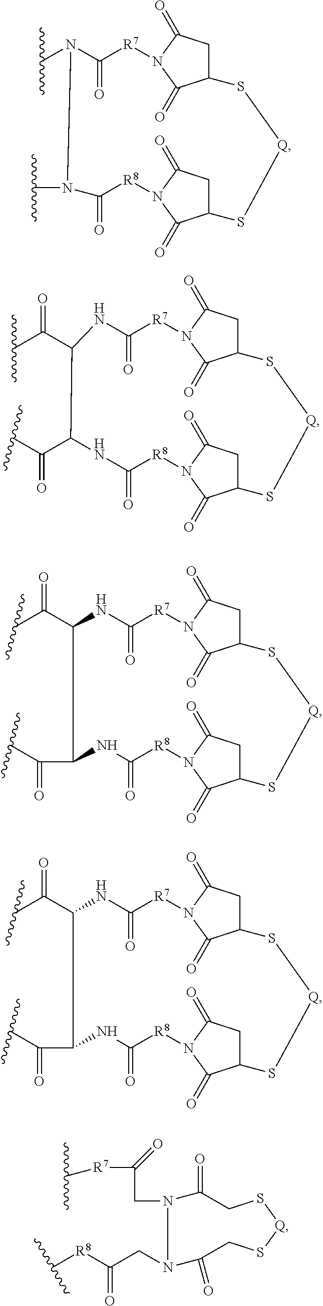

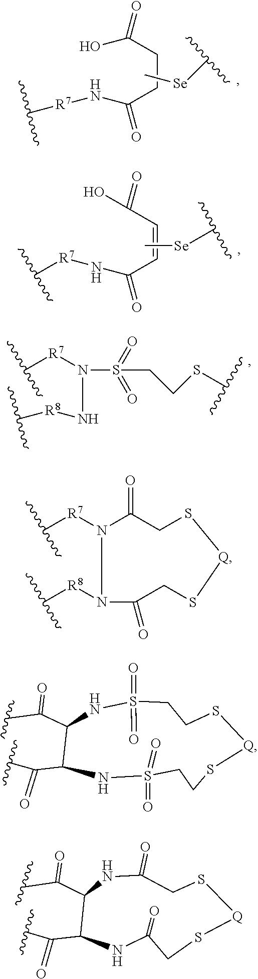

wherein R₇, R₈ and R₉ are independently selected from —C₁˜C₈ alkylene-, —C₁˜C₇ carbocyclo-, —O—(C₁˜C₈ alkyl)-, -arylene-, —C₁˜C₈ alkylene-arylene-, -arylene, —C₁˜C₈ alkylene-, —C₁˜C₈ alkylene-(C₁˜C₈ carbocyclo)-, —(C₃˜C₇ carbocyclo)-C₁˜C₈ alkylene-, —C₃˜C₈ heterocyclo-, —C₁˜C₈ alkylene-(C₃˜C₈ heterocyclo)-, —(C₃˜C₈ heterocyclo)-C₁˜C₉ alkylene-, —(CH₂CH₂O)ₖ—, —(CH(CH₃)CH₂O)ₖ—, and —(CH₂CH₂O)ₖ—CH₂—; k is an integer ranging from 1-30; X′″, Y′″ and Z′″ are independently selected from NH, O or S; Q, R₁ and R₂ are described above.

In a preferred embodiment, conjugates of the invention are antibody/cytotoxic agent, antibody fragment/cytotoxic agent, diabody/cytotoxic agent, tri(a)body/cytotoxic agent, epidermal growth factor (EGF)/cytotoxic agent, prostate specific membrane antigen (PSMA) inhibitor/cytotoxic agent, melanocyte stimulating hormone (MSH)/cytotoxic agent, thyroid stimulating hormone (TSH)/cytotoxic agent, polyclonal antibody/cytotoxic agent, somatostatin/cytotoxic agent, folate/cytotoxic agent, matriptase inhibitor/cytotoxic agent, estrogen/cytotoxic agent, estrogen analogue/cytotoxic agent, designed ankyrin repeat proteins (DARPins)/cytotoxic agent, androgen/cytotoxic agent, and androgen analogue/cytotoxic agent.

In a more preferred embodiment, cell binding molecule of the invention are monoclonal antibody. Examples of antibodies used for conjugation of cyotoxic agents in this prevention include, but are not limited to, 3F8 (anti-GD2), Abagovomab (anti CA-125), Abciximab (anti CD41 (integrin alpha-IIb), Adalimumab (anti-TNF-α), Adecatumumab (anti-EpCAM, CD326), Afelimomab (anti-TNF-α); Afutuzumab (anti-CD20), Alacizumab pegol (anti-VEGFR₂), ALD518 (anti-IL-6), Alemtuzumab (Campath, MabCampath, anti-CD52), Altumomab (anti-CEA), Anatumomab (anti-TAG-72), Anrukinzumab (IMA-638, anti-IL-13), Apolizumab (anti-HLA-DR), Arcitumomab (anti-CEA), Aselizumab (anti-L-selectin (CD62L), Atlizumab (tocilizumab, Actemra, RoActemra, anti-IL-6 receptor), Atorolimumab (anti-Rhesus factor), Bapineuzumab (anti-beta amyloid), Basiliximab (Simulect, antiCD25 (α chain of IL-2 receptor), Bavituximab (anti-phosphatidylserine), Bectumomab (LymphoScan, anti-CD22), Belimumab (Benlysta, LymphoStat-B, anti-BAFF), Benralizumab (anti-CD125), Bertilimumab (anti-CCL11 (eotaxin-1)), Besilesomab (Scintimun, anti-CEA-related antigen), Bevacizumab (Avastin, anti-VEGF-A), Biciromab (FibriScint, anti-fibrin II beta chain), Bivatuzumab (anti-CD44 v6), Blinatumomab (BiTE, anti-CD19), Brentuximab (cAC10, anti-CD30 TNFRSF8), Briakinumab (anti-IL-12, IL-23) Canakinumab (Ilaris, anti-IL-1), Cantuzumab (C242, anti-CanAg), Capromab, Catumaxomab (Removab, anti-EpCAM, anti-CD3), CC49 (anti-TAG-72), Cedelizumab (anti-CD4), Certolizumab pegol (Cimzia anti-TNF-α), Cetuximab (Erbitux, IMC-C225, anti-EGFR), Citatuzumab bogatox (anti-EpCAM), Cixutumumab (anti-IGF-1), Clenoliximab (anti-CD4), Clivatuzumab (anti-MUC1), Conatumumab (anti-TRAIL-R₂), CR6261 (anti-Influenza A hemagglutinin), Dacetuzumab (anti-CD40), Daclizumab (Zenapax, anti-CD25 (α chain of IL-2 receptor)), Daratumumab (anti-CD38 (cyclic ADP ribose hydrolase), Denosumab (Prolia, anti-RANKL), Detumomab (anti-B-lymphoma cell), Dorlimomab, Dorlixizumab, Ecromeximab (anti-GD3 ganglioside), Eculizumab (Soliris, anti-C5), Edobacomab (anti-endotoxin), Edrecolomab (Panorex, MAb17-1A, anti-EpCAM), Efalizumab (Raptiva, anti-LFA-1 (CD11a), Efungumab (Mycograb, anti-Hsp90), Elotuzumab (anti-SLAMF7), Elsilimomab (anti-IL-6), Enlimomab pegol (anti-ICAM-1 (CD54)), Epitumomab (anti-episialin), Epratuzumab (anti-CD22), Erlizumab (anti-ITGB2 (CD18)), Ertumaxomab (Rexomun, anti-HER2/neu, CD3), Etaracizumab (Abegrin, anti-integrin αᵥβ₃), Exbivirumab (anti-hepatitis B surface antigen), Fanolesomab (NeutroSpec, anti-CD15), Faralimomab (anti-interferon receptor), Farletuzumab (anti-folate receptor 1), Felvizumab (anti-respiratory syncytial virus), Fezakinumab (anti-IL-22), Figitumumab (anti-IGF-1 receptor), Fontolizumab (anti-IFN-γ), Foravirumab (anti-rabies virus glycoprotein), Fresolimumab (anti-TGF-β), Galiximab (anti-CD80), Gantenerumab (anti-beta amyloid), Gavilimomab (anti-CD147 (basigin)), Gemtuzumab (anti-CD33), Girentuximab (anti-carbonic anhydrase 9), Glembatumumab (CR011, anti-GPNMB), Golimumab (Simponi, anti-TNF-α), Gomiliximab (anti-CD23 (IgE receptor)), Ibalizumab (anti-CD4), Ibritumomab (anti-CD20), Igovomab (Indimacis-125, anti-CA-125), Imciromab (Myoscint, anti-cardiac myosin), Infliximab (Remicade, anti-TNF-α), Intetumumab (anti-CD51), Inolimomab (anti-CD25 (a chain of IL-2 receptor)), Inotuzumab (anti-CD22), Ipilimumab (anti-CD152), Iratumumab (anti-CD30 (TNFRSF8)), Keliximab (anti-CD4), Labetuzumab (CEA-Cide, anti-CEA), Lebrikizumab (anti-IL-β), Lemalesomab (anti-NCA-90 (granulocyte antigen)), Lerdelimumab (anti-TGF beta 2), Lexatumumab (anti-TRAIL-R₂), Libivirumab (anti-hepatitis B surface antigen), Lintuzumab (anti-CD33), Lucatumumab (anti-CD40), Lumiliximab (anti-CD23 (IgE receptor), Mapatumumab (anti-TRAIL-R₁), Maslimomab (anti-T-cell receptor), Matuzumab (anti-EGFR), Mepolizumab (Bosatria, anti-IL-5), Metelimumab (anti-TGF beta 1), Milatuzumab (anti-CD74), Minretumomab (anti-TAG-72), Mitumomab (BEC-2, anti-GD3 ganglioside), Morolimumab (anti-Rhesus factor), Motavizumab (Numax, anti-respiratory syncytial virus), Muromonab-CD3 (Orthoclone OKT3, anti-CD3), Nacolomab (anti-C242), Naptumomab (anti-5T4), Natalizumab (Tysabri, anti-integrin α₄), Nebacumab (anti-endotoxin), Necitumumab (anti-EGFR), Nerelimomab (anti-TNF-α), Nimotuzumab (Theracim, Theraloc, anti-EGFR), Nofetumomab, Ocrelizumab (anti-CD20), Odulimomab (Afolimomab, anti-LFA-1 (CD11a)), Ofatumumab (Arzerra, anti-CD20), Olaratumab (anti-PDGF-Rα), Omalizumab (Xolair, anti-IgE Fc region), Oportuzumab (anti-EpCAM), Oregovomab (OvaRex, anti-CA-125), Otelixizumab (anti-CD3), Pagibaximab (anti-lipoteichoic acid), Palivizumab (Synagis, Abbosynagis, anti-respiratory syncytial virus), Panitumumab (Vectibix, ABX-EGF, anti-EGFR), Panobacumab (anti-Pseudomonas aeruginosa), Pascolizumab (anti-IL-4), Pemtumomab (Theragyn, anti-MUC1), Pertuzumab (Omnitarg, 2C4, anti-HER2/neu), Pexelizumab (anti-C5), Pintumomab (anti-adenocarcinoma antigen), Priliximab (anti-CD4), Pritumumab (anti-vimentin), PRO 140 (anti-CCR5), Racotumomab (1E10, anti-(N-glycolylneuraminic acid (NeuGc, NGNA)-gangliosides GM3)), Rafivirumab (anti-rabies virus glycoprotein), Ramucirumab (anti-VEGFR2), Ranibizumab (Lucentis, anti-VEGF-A), Raxibacumab (anti-anthrax toxin, protective antigen), Regavirumab (anti-cytomegalovirus glycoprotein B), Reslizumab (anti-IL-5), Rilotumumab (anti-HGF), Rituximab (MabThera, Rituxanmab, anti-CD20), Robatumumab (anti-IGF-1 receptor), Rontalizumab (anti-IFN-α), Rovelizumab (LeukArrest, anti-CD11, CD18), Ruplizumab (Antova, anti-CD154 (CD40L)), Satumomab (anti-TAG-72), Sevirumab (anti-cytomegalovirus), Sibrotuzumab (anti-FAP), Sifalimumab (anti-IFN-α), Siltuximab (anti-IL-6), Siplizumab (anti-CD2), (Smart) MI95 (anti-CD33), Solanezumab (anti-beta amyloid), Sonepcizumab (anti-sphingosine-1-phosphate), Sontuzumab (anti-episialin), Stamulumab (anti-myostatin), Sulesomab (LeukoScan, (anti-NCA-90 (granulocyte antigen), Tacatuzumab (anti-alpha-fetoprotein), Tadocizumab (anti-integrin α_{IIb}β₃), Talizumab (anti-IgE), Tanezumab (anti-NGF), Taplitumomab (anti-CD19), Tefibazumab (Aurexis, (anti-clumping factor A), Telimomab, Tenatumomab (anti-tenascin C), Teneliximab (anti-CD40), Teplizumab (anti-CD3), TGN1412 (anti-CD28), Ticilimumab (Tremelimumab, (anti-CTLA-4), Tigatuzumab (anti-TRAIL-R2), TNX-650 (anti-IL-13), Tocilizumab (Atlizumab, Actemra, RoActemra, (anti-IL-6 receptor), Toralizumab (anti-CD154 (CD40L)), Tositumomab (anti-CD20), Trastuzumab (Herceptin, (anti-HER2/neu), Tremelimumab (anti-CTLA-4), Tucotuzumab celmoleukin (anti-EpCAM), Tuvirumab (anti-hepatitis B virus), Urtoxazumab (anti-Escherichia coli), Ustekinumab (Stelara, anti-IL-12, IL-23), Vapaliximab (anti-AOC3 (VAP-1)), Vedolizumab, (anti-integrin α₄β₇), Veltuzumab (anti-CD20), Vepalimomab (anti-AOC3 (VAP-1), Visilizumab (Nuvion, anti-CD3), Vitaxin (anti-vascular integrin avb3), Volociximab (anti-integrin α₅β₁), Votumumab (HumaSPECT, anti-tumor antigen CTAA16.88), Zalutumumab (HuMax-EGFr, (anti-EGFR), Zanolimumab (HuMax-CD4, anti-CD4), Ziralimumab (anti-CD147 (basigin)), Zolimomab (anti-CD5), Etanercept (Enbrel®), Alefacept (Amevive®), Abatacept (Orencia®), Rilonacept (Arcalyst), 14F7 [anti-IRP-2 (Iron Regulatory Protein 2)], 14G2a (anti-GD2 ganglioside, from Nat. Cancer Inst. for melanoma and solid tumors), J591 (anti-PSMA, Weill Cornell Medical School for prostate cancers), 225.285 [anti-HMW-MAA (High molecular weight-melanoma-associated antigen), Sorin Radiofarmaci S.R.L. (Milan, Italy) for melanoma], COL-1 (anti-CEACAM3, CGM1, from Nat. Cancer Inst. USA for colorectal and gastric cancers), CYT-356 (Oncoltad®, for prostate cancers), HNK20 (OraVax Inc. for respiratory syncytial virus), ImmuRAIT (from Immunomedics for NHL), Lym-1 (anti-HLA-DR10, Peregrine Pharm. for Cancers), MAK-195F [anti-TNF (tumor necrosis factor; TNFA, TNF-alpha; TNFSF2), from Abbott/Knoll for Sepsis toxic shock], MEDI-500 [T10B9, anti-CD3, TRαβ (T cell receptor alpha/beta), complex, from MedImmune Inc for Graft-versus-host disease], RING SCAN [anti-TAG 72 (tumour associated glycoprotein 72), from Neoprobe Corp. for Breast, Colon and Rectal cancers], Avicidin (anti-EPCAM (epithelial cell adhesion molecule), anti-TACSTD1 (Tumor-associated calcium signal transducer 1), anti-GA733-2 (gastrointestinal tumor-associated protein 2), anti-EGP-2 (epithelial glycoprotein 2); anti-KSA; KS1/4 antigen; M4S; tumor antigen 17-1A; CD326, from NeoRx Corp. for Colon, Ovarian, Prostate cancers and NHL]; LymphoCide, Smart ID10, Oncolym, Allomune, anti-VEGF, CEAcide, IMC-1C11, and Cetuximab.

Other antibodies as binding ligands include, but are not limited to, are antibodies against the following antigens: Aminopeptidase N (CD13), Annexin A1, B7-H3 (CD276, various cancers), CA125 (ovarian), CA15-3 (carcinomas), CA19-9 (carcinomas), L6 (carcinomas), Lewis Y (carcinomas), Lewis X (carcinomas), alpha fetoprotein (carcinomas), CA242 (colorectal), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (Metastatic melanoma, sarcoma), CD52 (leukemia), CD56 (small cell lung cancers, ovarian cancer, Merkel cell carcinoma, and the liquid tumor, multiple myeloma), CD66e (cancers), CD70 (metastatic renal cell carcinoma and non-Hodgkin lymphoma), CD74 (multiple myeloma), CD80 (lymphoma), CD98 (cancers), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (carcinoembryonic antigen; CEA, CD66e) (breast, colorectal and lung cancers), DLL4 (Δ-like-4), EGFR (Epidermal Growth Factor Receptor, various cancers), CTLA4 (melanoma), CXCR4 (CD184, Heme-oncology, solid tumors), Endoglin (CD105, solid tumors), EPCAM (epithelial cell adhesion molecule, bladder, head, neck, colon, NHL prostate, and ovarian cancers), ERBB2 (Epidermal Growth Factor Receptor 2; lung, breast, prostate cancers), FCGR1 (autoimmune diseases), FOLR (folate receptor, ovarian cancers), GD2 ganglioside (cancers), G-28 (a cell surface antigen glyvolipid, melanoma), GD3 idiotype (cancers), Heat shock proteins (cancers), HER1 (lung, stomach cancers), HER2 (breast, lung and ovarian cancers), HLA-DR10 (NHL), HLA-DRB (NHL, B cell leukemia), human chorionic gonadotropin (carcinoma), IGF1R (insulin-like growth factor 1 receptor, solid tumors, blood cancers), IL-2 receptor (interleukin 2 receptor, T-cell leukemia and lymphomas), IL-6R (interleukin 6 receptor, multiple myeloma, RA, Castleman's disease, IL6 dependent tumors), Integrins (αvβ3, α5β1, α6β4, αllβ3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 or MUC1-KLH (breast, ovarian, cervix, bronchus and gastrointestinal cancer), MUC16 (CA125) (Ovarian cancers), CEA (colorectal), gp100 (melanoma), MART1 (melanoma), MPG (melanoma), MS4A1 (membrane-spanning 4-domains subfamily A, small cell lung cancers, NHL), Nucleolin, Neu oncogene product (carcinomas), P21 (carcinomas), Paratope of anti-(N-glycolylneuraminic acid, Breast, Melanoma cancers), PLAP-like testicular alkaline phosphatase (ovarian, testicular cancers), PSMA (prostate tumors), PSA (prostate), ROBO4, TAG 72 (tumour associated glycoprotein 72, AML, gastric, colorectal, ovarian cancers), T cell transmembrane protein (cancers), Tie (CD202b), TNFRSF10B (tumor necrosis factor receptor superfamily member 10B, cancers), TNFRSF13B (tumor necrosis factor receptor superfamily member 13B, multiple myeloma, NHL, other cancers, RA and SLE), TPBG (trophoblast glycoprotein, Renal cell carcinoma), TRAIL-R1 (Tumor necrosis apoprosis Inducing ligand Receptor 1, lymphoma, NHL, colorectal, lung cancers), VCAM-1 (CD106, Melanoma), VEGF, VEGF-A, VEGF-2 (CD309) (various cancers). Some other tumor associated antigens recognized by antibodies have been reviewed (Gerber, et al, mAbs 1:3, 247-253 (2009); Novellino et al, Cancer Immunol Immunother. 54(3), 187-207 (2005). Franke, et al, Cancer Biother Radiopharm. 2000, 15, 459-76). Examples of these antigens that antibodies against are: Many other Cluster of Differentiations (CD2, CD2R, CD3, CD3gd, CD3e, CD4, CD5, CD6, CD7, CD8, CD8a, CD8b, CD9, CD10, CD11a, CD11b, CD11c, CD12, CD12w, CD13, CD14, CD15, CD15s, CD15u, CD16, CD16a, CD16b, CD17, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD42a, CD42b, CD42c, CD42d, CD43, CD44, CD44R, CD45, CD45RA, CD45RB, CD45RO, CD46, CD47, CD47R, CD48, CD49a, CD49b, CD49c, CD49e, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD74, CD75, CD75s, CD76, CD77, CD78, CD79, CD79a, CD79b, CD80, CD81, CD82, CD83, CD84, CDw84, CD85, CD86, CD87, CD88, CD89, CD90, CD91, CD92, CDw92, CD93, CD94, CD95, CD96, CD97, CD98, CD99, CD99R, CD100, CD101, CD102, CD103, CD104, CD105, CD106, CD107, CD107a, CD107b, CD108, CD109, CD110, CD111, CD112, CD113, CDw113, CD114, CD115, CD116, CD117, CD118, CD119, CDw119, CD120a, CD120b, CD121a, CD121b, CDw121b, CD122, CD123, CDw123, CD124, CD125, CDw125, CD126, CD127, CD128, CDw128, CD129, CD130, CD131, CDw131, CD132, CD133, CD134, CD135, CD136, CDw136, CD137, CDw137, CD138, CD139, CD140a, CD140b, CD141, CD142, CD143, CD144, CD145, CDw145, CD146, CD147, CD148, CD149, CD150, CD151, CD152, CD153, CD154, CD155, CD156a, CD156b, CDw156c, CD157, CD158a, CD158b, CD159a, CD159b, CD159c, CD160, CD161, CD162, CD162R, CD163, CD164, CD165, CD166, CD167, CD167a, CD168, CD169, CD170, CD171, CD172a, CD172b, CD172g, CD173, CD174, CD175, CD175s, CD176, CD177, CD178, CD179, CD180, CD181, CD182, CD183, CD184, CD185, CD186, CDw186, CD187, CD188, CD189, CD190, Cd191, CD192, CD193, CD194, CD195, CD196, CD197, CD198, CDw98, CD199, CDw199, CD200, CD200a, CD200b, CD201, CD202, CD202b, CD203, CD203c, CD204, CD205, CD206, CD207, CD208, CD209, CD210, CDw210, CD212, CD213a1, CD213a2, CDw217, CDw218a, CDw218b, CD220, CD221, CD222, CD223, CD224, CD225, CD226, CD227, CD228, CD229, CD230, CD231, CD232, CD233, CD234, CD235a, CD235ab, CD235b, CD236, CD236R, CD238, CD239, CD240, CD240CE, CD240D, CD241, CD242, CD243, CD244, CD245, CD246, CD247, CD248, CD249, CD252, CD253, CD254, CD256, CD257, CD258, CD261, CD262, CD263, CD265, CD266, CD267, CD268, CD269, CD271, CD273, CD274, CD275, CD276 (B7-H3), CD277, CD278, CD279, CD280, CD281, CD282, CD283, CD284, CD289, CD292, CDw293, CD294, CD295, CD296, CD297, CD298, CD299, CD300a, CD300c, CD300e, CD301, CD302, CD303, CD304, CD305, CD306, CD309, CD312, CD314, CD315, CD316, CD317, CD318, CD319, CD320, CD321, CD322, CD324, CDw325, CD326, CDw327, CDw328, CDw329, CD331, CD332, CD333, CD334, CD335, CD336, CD337, CDw338, CD339), Annexin A1, Nucleolin, Endoglin (CD105), ROBO4, Amino-peptidase N, Δ-like-3 (DLL3), Δ-like-4 (DLL4), VEGFR-2 (CD309), CXCR4 9CD184), Tie2, B7-H3, WT1, MUC1, LMP2, HPV E6 E7, EGFRvIII, HER-2/neu, Idiotype, MAGE A3, p53 nonmutant, NY-ESO-1, GD2, CEA, MelanA/MART1, Ras mutant, gp100, p53 mutant, Proteinase3 (PR1), bcr-abl, Tyrosinase, Survivin, hTERT, Sarcoma translocation breakpoints, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, Fos-related antigen 1.

Production of antibodies used in the present invention involves in vivo or in vitro procedures or combinations thereof. Methods for producing polyclonal anti-receptor peptide antibodies are well-known in the art, such as in U.S. Pat. No. 4,493,795 (to Nestor et al). A monoclonal antibody is typically made by fusing myeloma cells with the spleen cells from a mouse that has been immunized with the desired antigen (Köhler, G.; Milstein, C. (1975). Nature 256: 495-497). The detailed procedures are described in “Antibodies—A Laboratory Manual”, Harlow and Lane, eds., Cold Spring Harbor Laboratory Press, New York (1988), which is incorporated herein by reference. Particularly monoclonal antibodies are produced by immunizing mice, rats, hamsters or any other mammal with the antigen of interest such as the intact target cell, antigens isolated from the target cell, whole virus, attenuated whole virus, and viral proteins. Splenocytes are typically fused with myeloma cells using polyethylene glycol (PEG) 6000. Fused hybrids are selected by their sensitivity to HAT (hypoxanthine-aminopterin-thymine). Hybridomas producing a monoclonal antibody useful in practicing this invention are identified by their ability to immunoreact specified receptors or inhibit receptor activity on target cells.

A monoclonal antibody used in the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques, such as using protein-A affinity chromatography; anion, cation, hydrophobic, or size exclusive chromatographies (particularly by affinity for the specific antigen after Protein A, and sizing column chromatography); centrifugation, differential solubility, or by any other standard technique for the purification of proteins.

Media useful for the preparation of these compositions are both well-known in the art and commercially available and include synthetic culture media. An exemplary synthetic medium is Dulbecco's minimal essential medium (DMEM; Dulbecco et al., Virol. 8:396 (1959)) supplemented with 4.5 gm/l glucose, 20 mm glutamine, 20% fetal calf serum and with an anti-foaming agent, such as polyoxyethylene-polyoxypropylene block copolymer.

In addition, antibody-producing cell lines can also be created by techniques other than fusion, such as direct transformation of B lymphocytes with oncogenic DNA, or transfection with an oncovirus, such as Epstein-Barr virus (EBV, also called human herpesvirus 4 (HHV-4)) or Kaposi's sarcoma-associated herpesvirus (KSHV). See, U.S. Pat. Nos. 4,341,761; 4,399,121; 4,427,783; 4,444,887; 4,451,570; 4,466,917; 4,472,500; 4,491,632; 4,493,890. A monoclonal antibody may also be produced via an anti-receptor peptide or peptides containing the carboxyl terminal as described well-known in the art. See Niman et al., Proc. Natl. Acad. Sci. USA, 80: 4949-4953 (1983); Geysen et al., Proc. Natl. Acad. Sci. USA, 82: 178-182 (1985); Lei et al. Biochemistry 34(20): 6675-6688, (1995). Typically, the anti-receptor peptide or a peptide analog is used either alone or conjugated to an immunogenic carrier, as the immunogen for producing anti-receptor peptide monoclonal antibodies.

There are also a number of other well-known techniques for making monoclonal antibodies as binding molecules in this invention. Particularly useful are methods of making fully human antibodies. One method is phage display technology which can be used to select a range of human antibodies binding specifically to the antigen using methods of affinity enrichment. Phage display has been thoroughly described in the literature and the construction and screening of phage display libraries are well known in the art, see, e.g., Dente et al, Gene. 148(1):7-13 (1994); Little et al, Biotechnol Adv. 12(3):539-55 (1994); Clackson et al., Nature 352:264-628 (1991); Huse et al., Science 246:1275-1281 (1989), Hoogenboom et al. in Methods in Molecular Biology 178:1-37 (2001) (O'Brien et al., ed., Human Press, Totowa, N.J.), and in certain embodiments, in Lee et al. J. Mol. Biol. 340:1073-1093 (2004).

Moncolonal antibodies derived by hybridoma technique from another species than human, such as mouse, can be humanized to avoid human anti-mouse antibodies when infused into humans. Among the more common methods of humanization of antibodies are complementarity-determining region grafting and resurfacing. These methods have been extensively described, see e.g. U.S. Pat. Nos. 5,859,205 and 6,797,492; Liu et al, Immunol Rev. 222:9-27 (2008); Almagro et al, Front Biosci. 1; 13:1619-33 (2008); Lazar et al, Mol Immunol. 44(8):1986-98 (2007); Li et al, Proc. Natl. Acad. Sci. USA. 103(10):3557-62 (2006) each incorporated herein by reference. Fully human antibodies can also be prepared by immunizing transgenic mice, rabbits, monkeys, or other mammals, carrying large portions of the human immunoglobulin heavy and light chains, with an immunogen. Examples of such mice are: the Xenomouse. (Abgenix, Inc.), the HuMAb-Mouse (Medarex/BMS), the VelociMouse (Regeneron), see also U.S. Pat. Nos. 6,596,541, 6,207,418, 6,150,584, 6,111,166, 6,075,181, 5,922,545, 5,661,016, 5,545,806, 5,436,149 and 5,569,825. In human therapy, murine variable regions and human constant regions can also be fused to construct called “chimeric antibodies” that are considerably less immunogenic in man than murine mAbs (Kipriyanov et al, Mol Biotechnol. 26:39-60 (2004); Houdebine, Curr Opin Biotechnol. 13:625-9 (2002) each incorporated herein by reference). In addition, site-directed mutagenesis in the variable region of an antibody can result in an antibody with higher affinity and specificity for its antigen (Brannigan et al, Nat Rev Mol Cell Biol. 3:964-70, (2002)); Adams et al, J Immunol Methods. 231:249-60 (1999)) and exchanging constant regions of a mAb can improve its ability to mediate effector functions of binding and cytotoxicity.

Antibodies immunospecific for a malignant cell antigen can also be obtained commercially or produced by any method known to one of skill in the art such as, e.g., chemical synthesis or recombinant expression techniques. The nucleotide sequence encoding antibodies immunospecific for a malignant cell antigen can be obtained commercially, e.g., from the GenBank database or a database like it, the literature publications, or by routine cloning and sequencing.

DNA encoding hybridoma-derived monoclonal antibodies or phage display Fv clones of the antibody can be readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells (Skerra et al., Curr. Opinion in Immunol., 5: 256 (1993) and Pluckthun, Immunol. Revs, 130: 151 (1992)). Antibodies can also be produced by using an expression system in which the quantitative ratio of expressed polypeptide components can be modulated in order to maximize the yield of secreted and properly assembled antibodies. Such modulation is accomplished at least in part by simultaneously modulating translational strengths for the polypeptide components. After the fermentation which is known in the art, the produced antibody protein is further purified to obtain preparations that are substantially homogeneous for further assays and uses. Standard protein purification methods known in the art can be employed. The exemplary purification procedures: fractionation on immunoaffinity (such as Protein A columns) or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

Apart from an antibody, a peptide or protein that bind/block/target or in some other way interact with the epitopes or corresponding receptors on a targeted cell can be used as a binding molecule. These peptides or proteins could be any random peptide or proteins that have an affinity for the epitopes or corresponding receptors and they don't necessarily have to be of the immunoglobulin family. These peptides can be isolated by similar techniques as for phage display antibodies (Szardenings, J Recept Signal Transduct Res. 2003; 23(4):307-49). The use of peptides from such random peptide libraries can be similar to antibodies and antibody fragments. The binding molecules of peptides or proteins may be conjugated on or linked to a large molecules or materials, such as, but is not limited, an albumin, a polymer, a liposome, a nano particle, as long as such attachment permits the peptide or protein to retain its antigen binding specificity.

The cell-binding molecule/ligands or cell receptor agonists can be Ig-based and non-Ig-based protein scaffold molecules. The Ig-Based scaffolds can be selected, but not limited, from Nanobody (a derivative of VHH (camelid Ig)) (Muyldermans S., 2013 Annu Rev Biochem. 82, 775-97); Domain antibodies (dAb, a derivative of VH or VL domain) (Holt, L. J, et al, 2003, Trends Biotechnol. 21, 484-90); Bispecific T cell Engager (BiTE, a bispecific diabody) (Baeuerle, P. A, et al, 2009, Curr. Opin. Mol. Ther. 11, 22-30); Dual Affinity ReTargeting (DART, a bispecific diabody) (Moore P. A. P, et al. 2011, Blood 117(17), 4542-51); Tetravalent tandem antibodies (TandAb, a dimerized bispecific diabody) (Cochlovius, B, et al. 2000, Cancer Res. 60(16):4336-4341). The Non-Ig scaffolds can be selected, but not limited, from Anticalin (a derivative of Lipocalins) (Skerra A. 2008, FEBS J., 275(11): 2677-83; Beste G, et al, 1999 Proc. Nat. Acad. USA. 96(5):1898-903; Skerra, A. 2000 Biochim Biophys Acta, 1482(1-2): 337-50; Skerra, A. 2007, Curr Opin Biotechnol. 18(4): 295-304; Skerra, A. 2008, FEBS J. 275(11):2677-83); Adnectins (10th FN3 (Fibronectin)) (Koide, A, et al, 1998 J. Mol. Biol., 284(4):1141-51; Batori V, 2002, Protein Eng. 15(12): 1015-20; Tolcher, A. W, 2011, Clin. Cancer Res. 17(2): 363-71; Hackel, B. J, 2010, Protein Eng. Des. Sel. 23(4): 211-19); Designed Ankyrin Repeat Proteins (DARPins) (a derivative of ankrin repeat (AR) proteins) (Boersma, Y. L, et al, 2011 Curr Opin Biotechnol. 22(6): 849-57), e.g. DARPin C9, DARPin Ec4 and DARPin E69_LZ3_E01 (Winkler J, et al, 2009 Mol Cancer Ther. 8(9), 2674-83; Patricia M-K. M., et al, Clin Cancer Res. 2011; 17(1):100-10; Boersma Y. L, et al, 2011 J. Biol. Chem. 286(48), 41273-85); Avimers (a domain A/low-density lipoprotein (LDL) receptor) (Boersma Y. L, 2011 J. Biol. Chem. 286(48): 41273-41285; Silverman J, et al, 2005 Nat. Biotechnol., 23(12):1556-61).

Examples of the small molecule structures of the cell-binding molecules/ligands or cell receptor agonists of the patent application are the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αᵥβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor,), LB23 (for an androgen receptor), LB24 (Cilengitide/cyclo(-RGDfV-) for an αᵥ intergrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a neucleoside analog), LB53 (Erlotinib analog) and LB54 (Lapatinib analog) which are shown in the following structures:

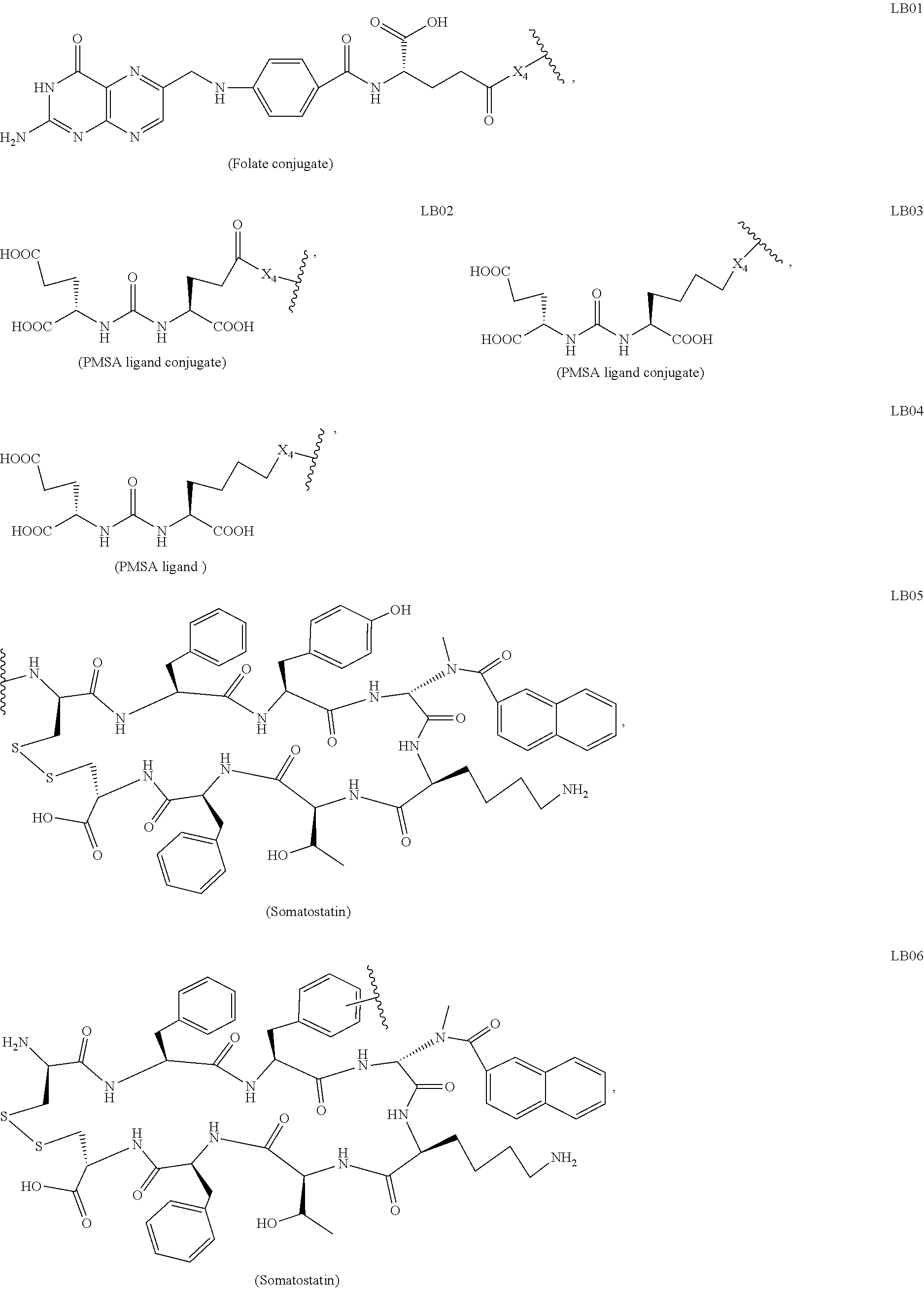

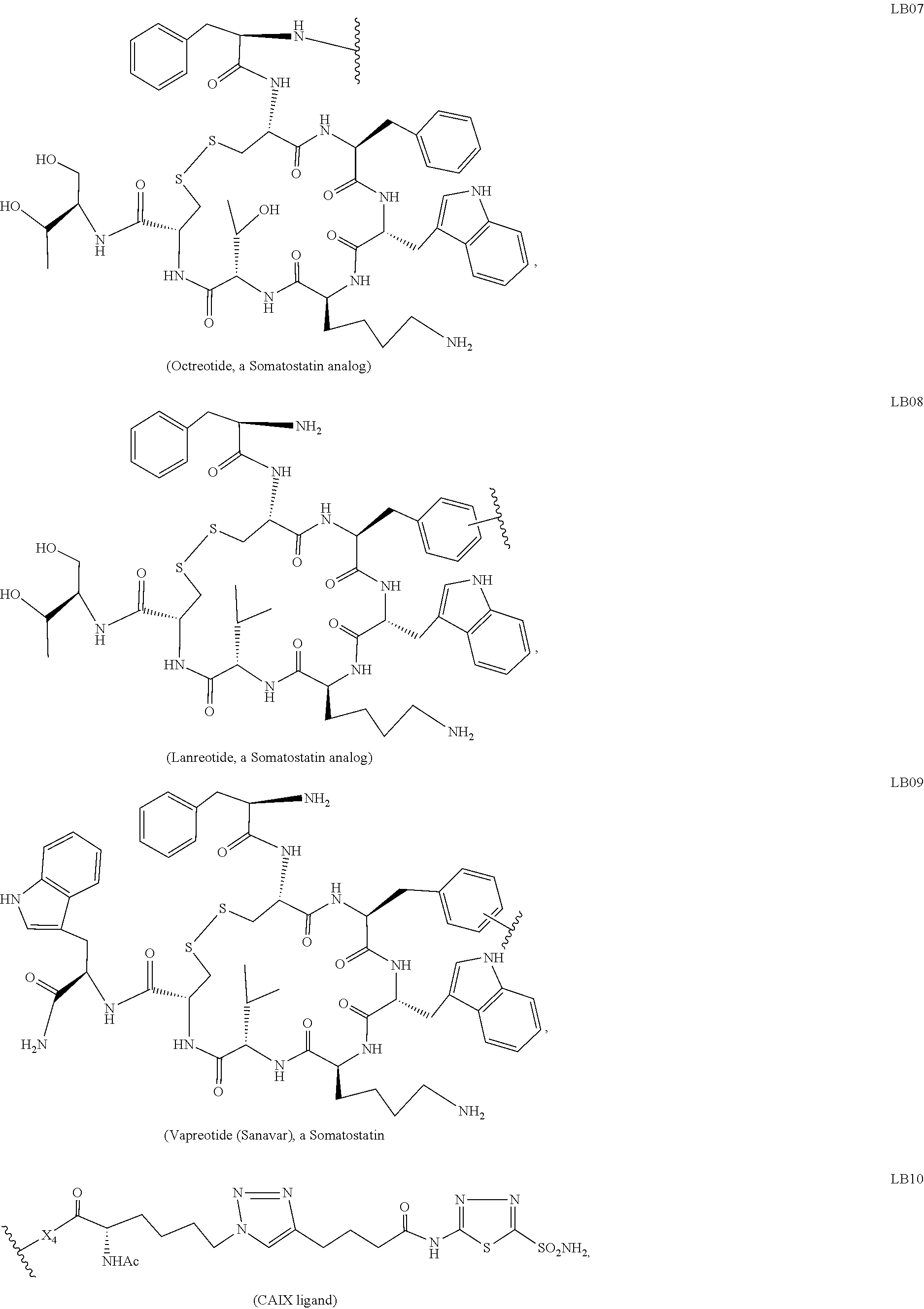

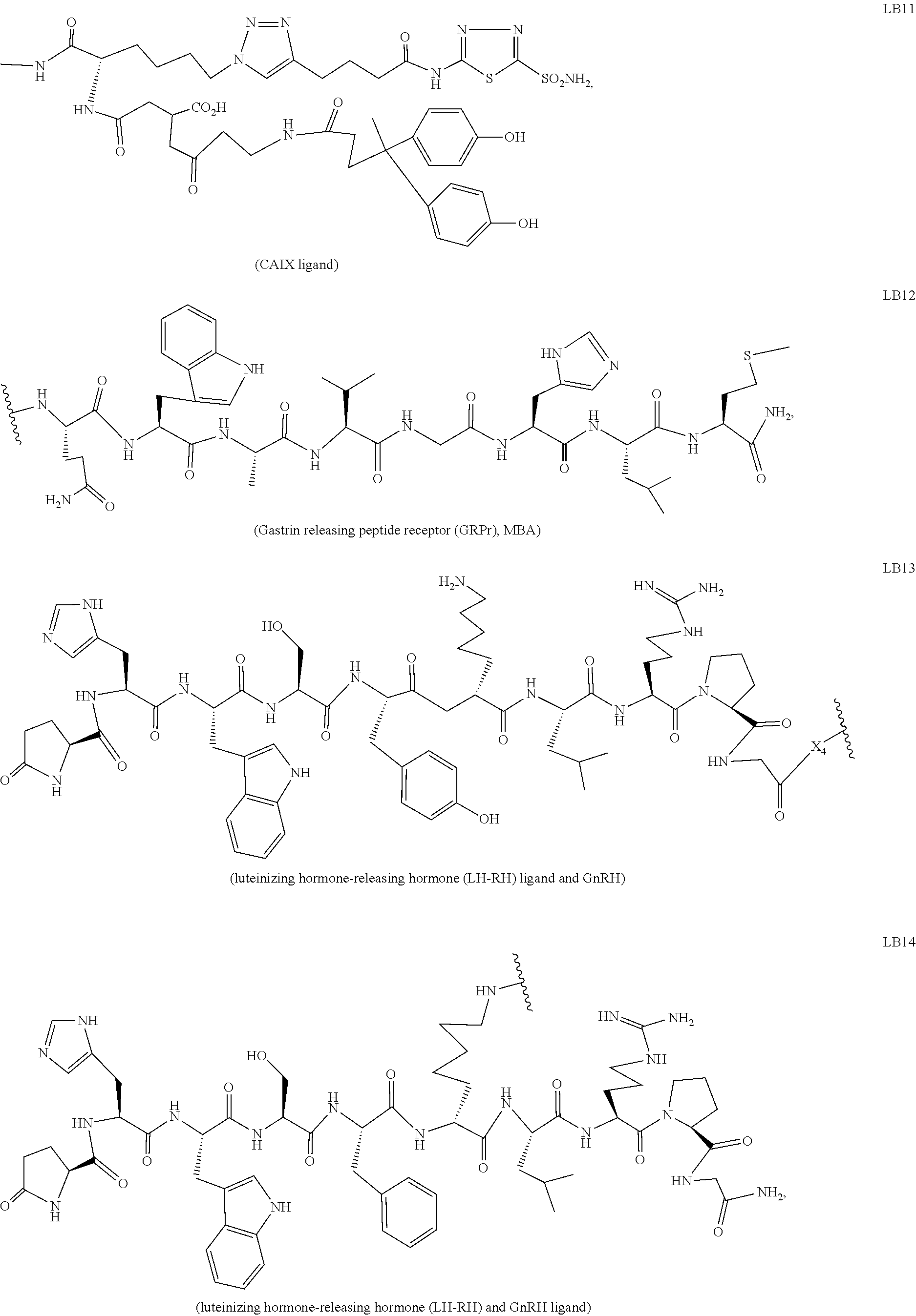

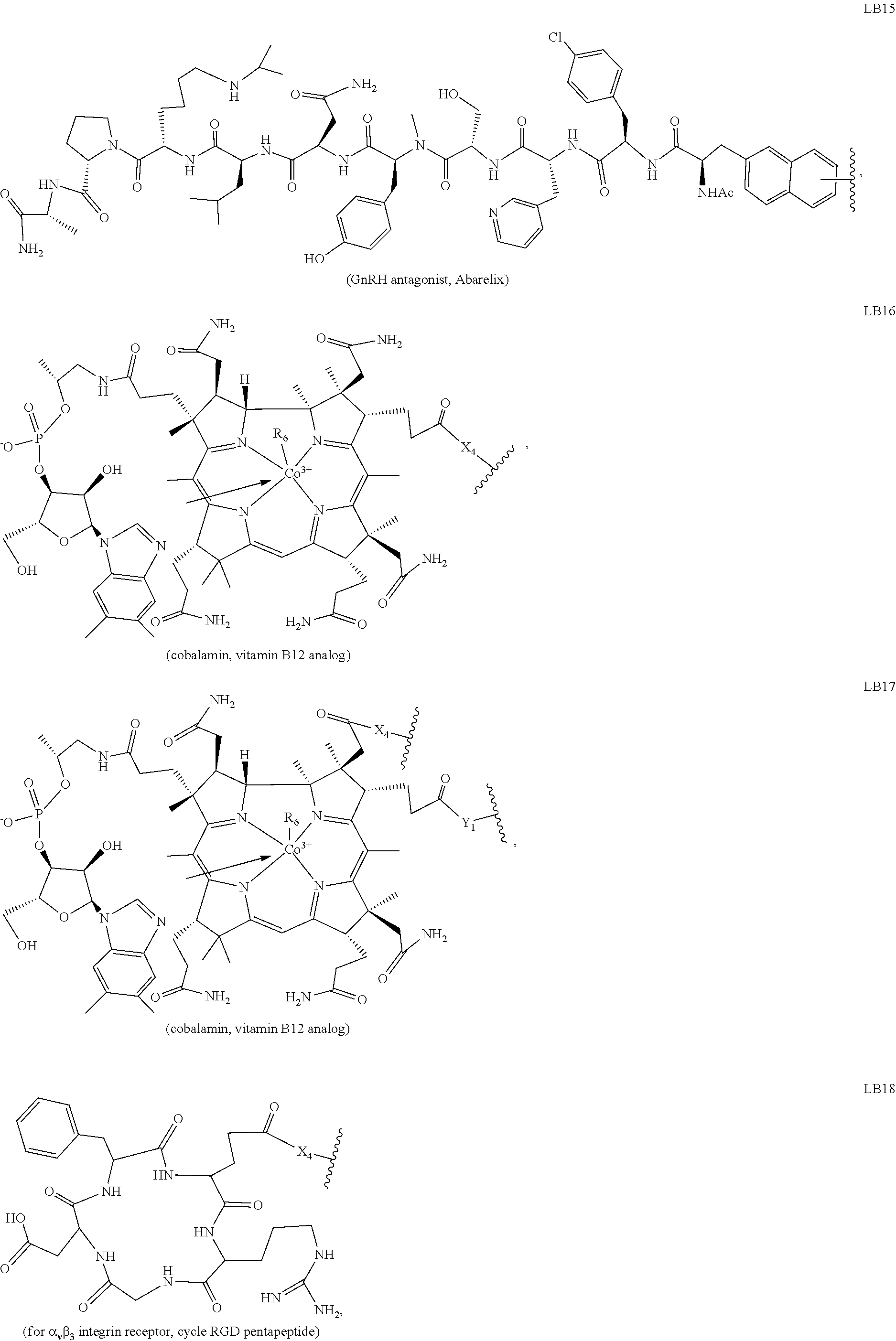

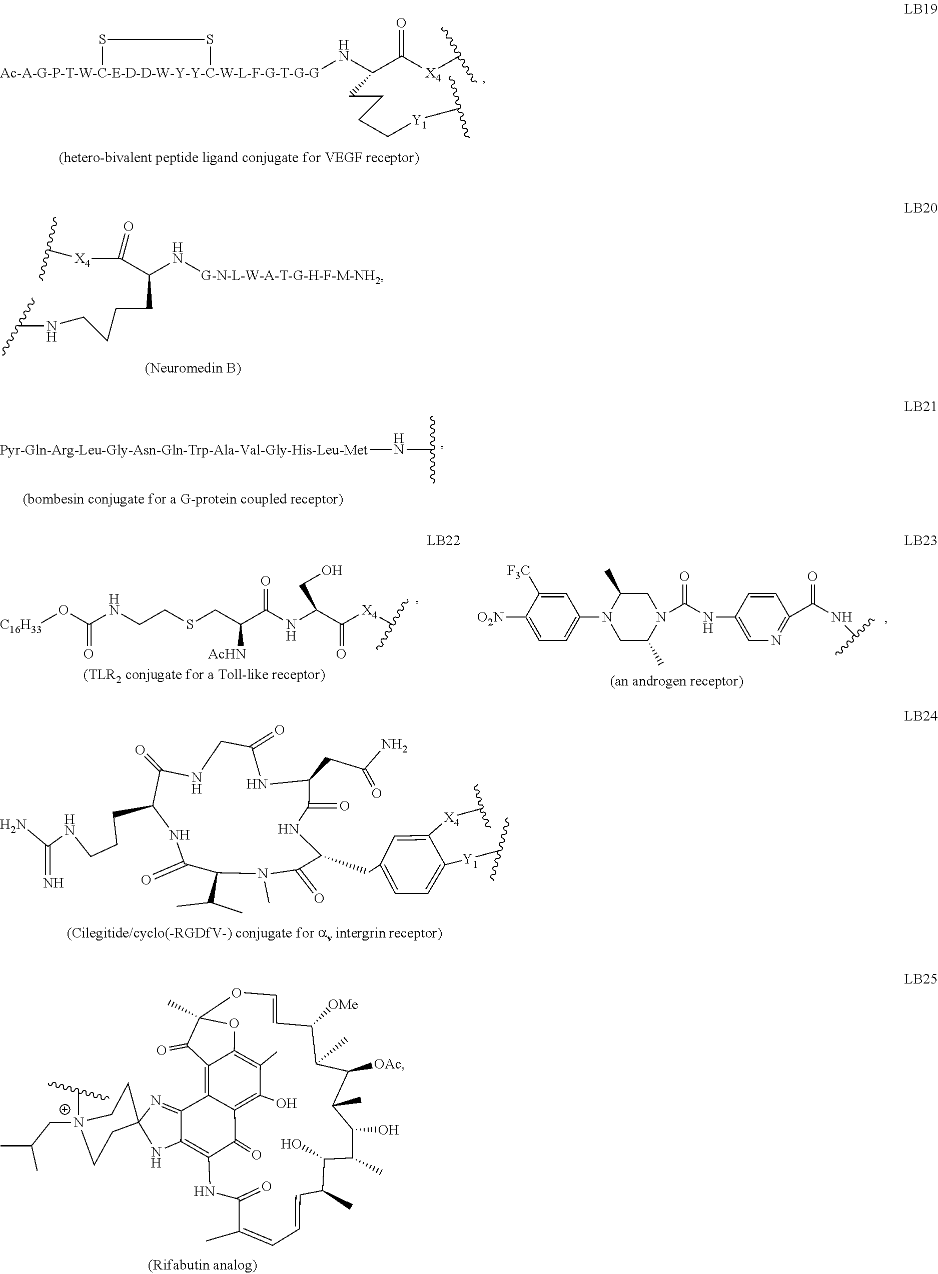

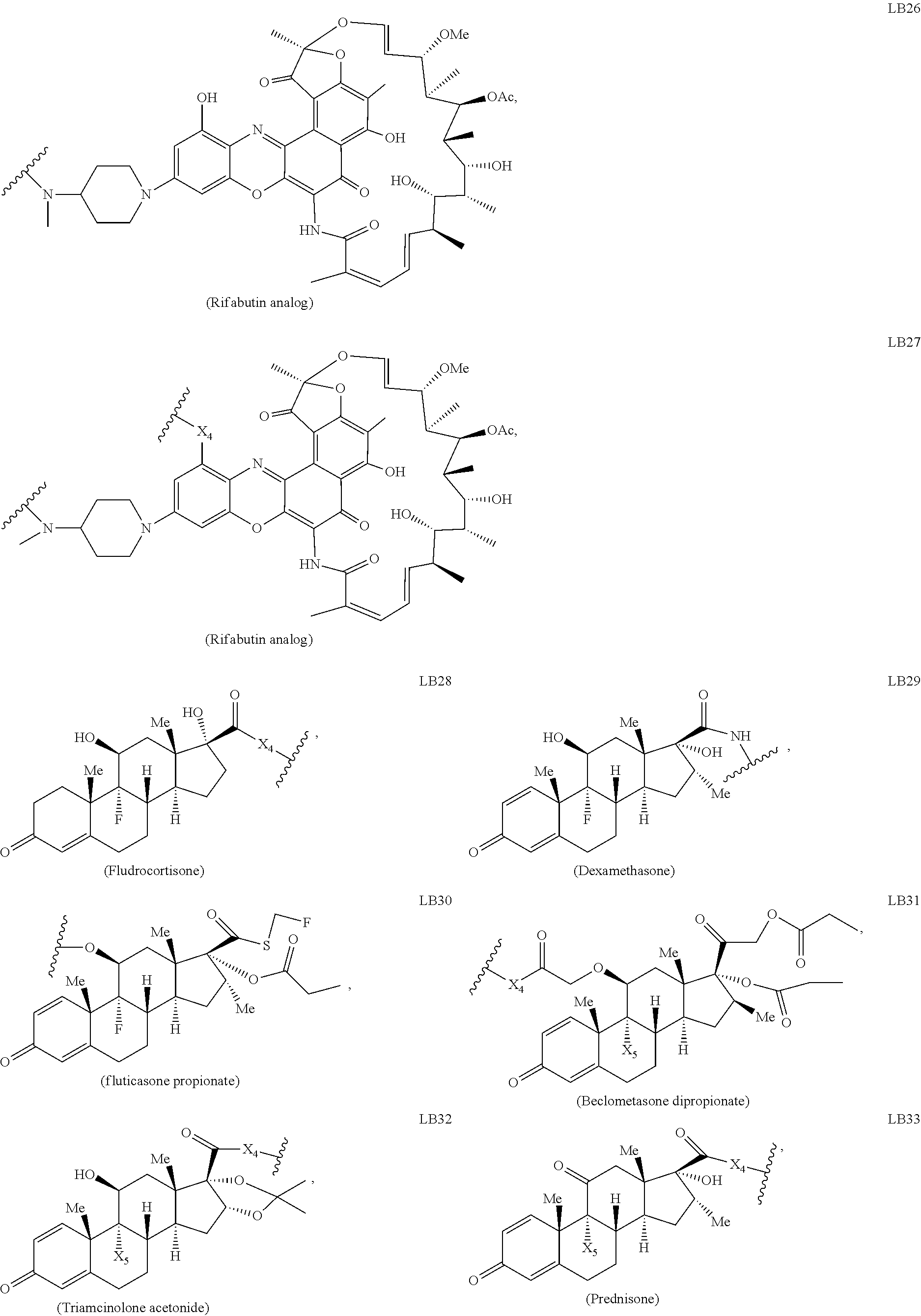

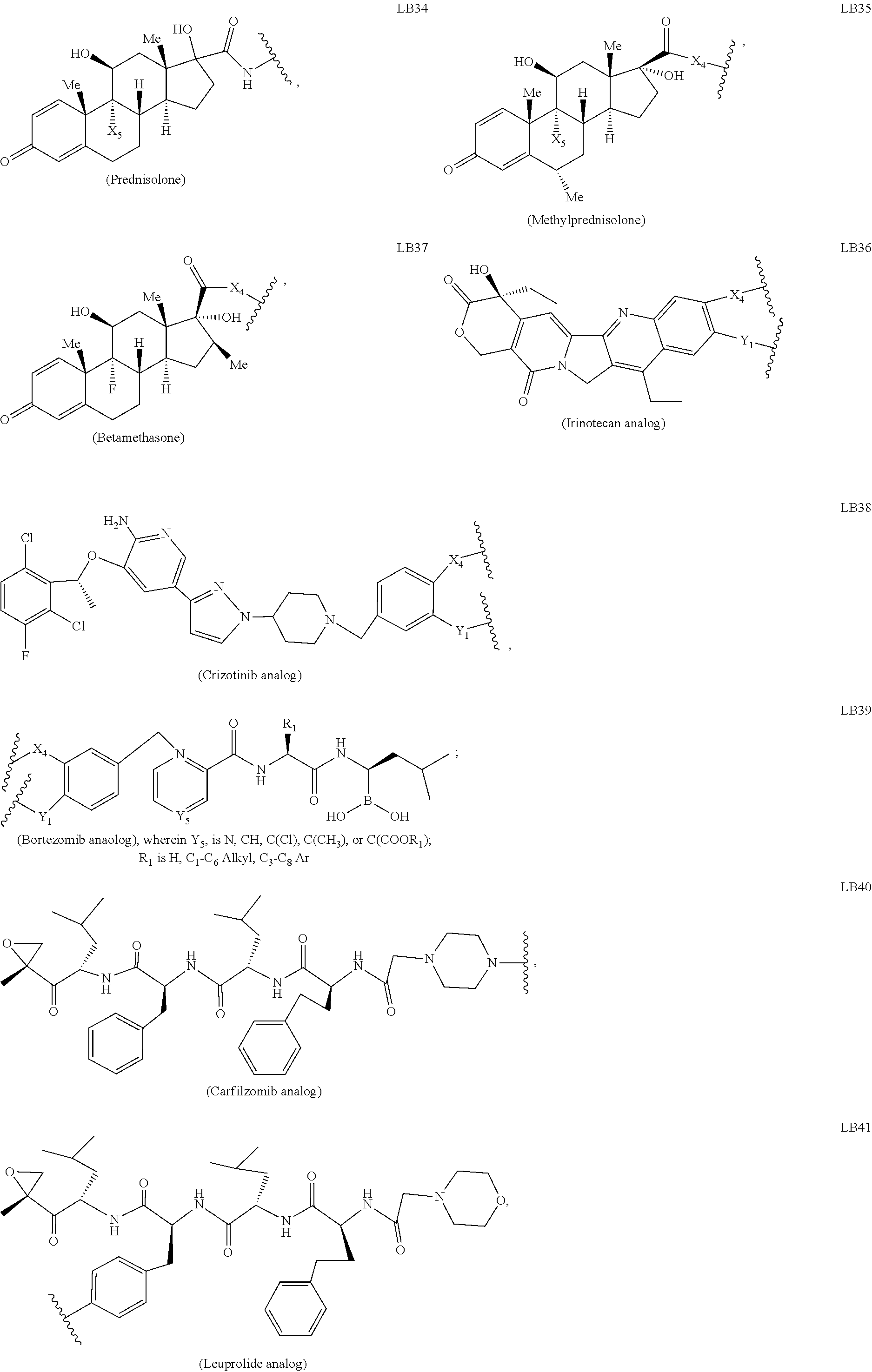

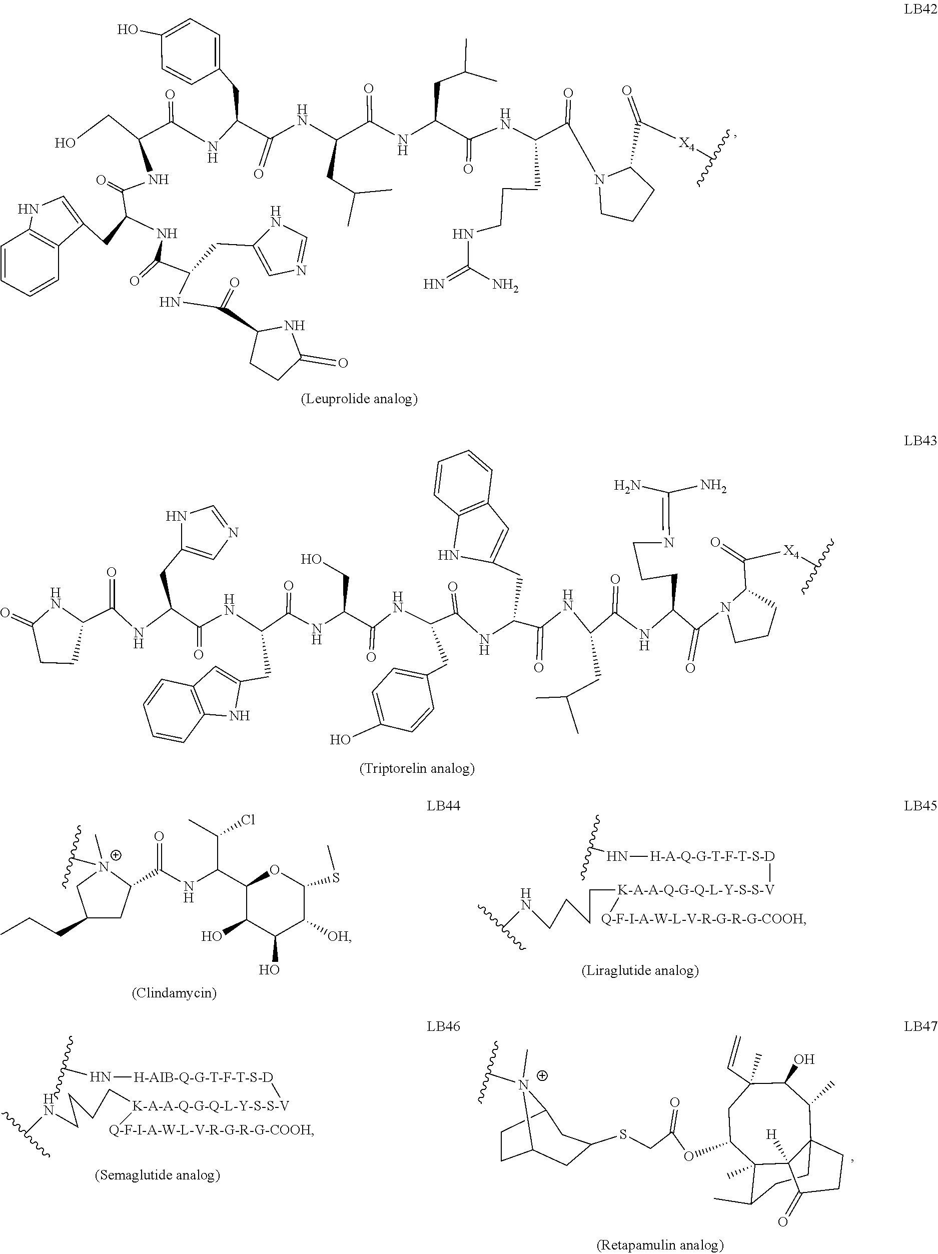

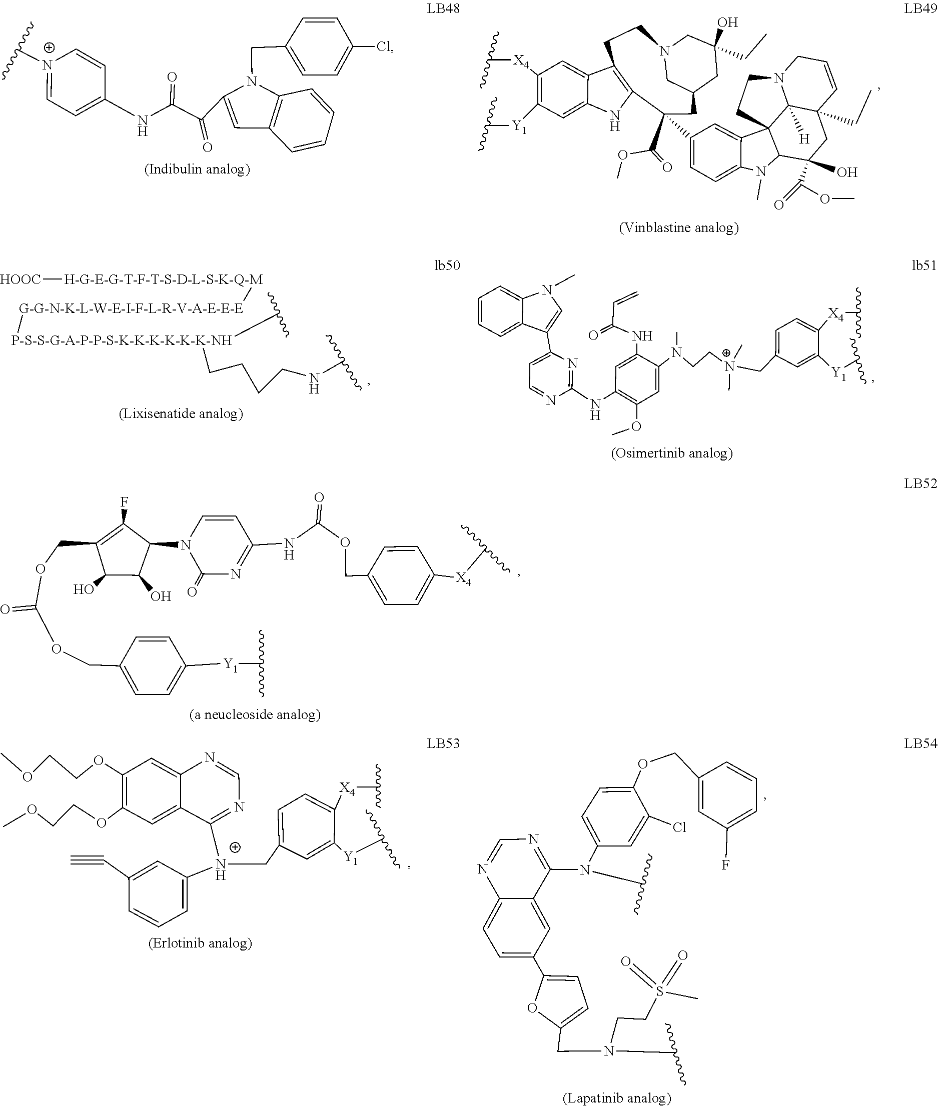

wherein [symbol] is the site to link the side chain linker of the present patent; X₄, and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁; X₁ is H, CH₂, OH, O, C(O), C(O)NH, C(O)N(R₁), R₁, NHR₁, NR₁, C(O)R₁ or C(O)O; X₅ is H, CH₃, F, or Cl; M₁ and M₂ are independently H, Na, K, Ca, Mg, NH₄, N(R₁R₂R₃R₄); R₁, R₂, R₃ and R₄ are defined in Formula (I);

Any one of several different reactive groups on a cell binding agent, preferably on an antibody, can be a conjugation site, such as F-amino groups in lysine residues, pendant carbohydrate moieties, carboxylic acid groups, disulfide groups, and thiol groups. For reviews on antibody reactive groups suitable for conjugation, see, e.g., Hermanson, G. T. (2008). Bioconjugate Techniques, Academic Press; Garnett, Adv. Drug Delivery Rev. 53 (2001), 171-216 and Dubowchik and Walker, Pharmacology & Therapeutics 83 (1999), 67-123, the disclosures of which are incorporated herein by reference.

The cytotoxic agents, cross-linked PBD dimers of this invention can be directly conjugated (linked) to a cell binding agent, or via a bifunctional linker or a cross-linking agent to a cell binding agent. The bifunctional linker possess two reactive groups; one of which is capable of reacting with a cell binding agent while the other one reacts with one or more molecules of cytotoxic agent of the invention. The bifunctional crosslinkers are well known in the art (see, for example, U.S. Pat. No. 5,208,020; Isalm and Dent in Bioconjugation chapter 5, p 218-363, Groves Dictionaries Inc. New York, 1999). Examples of bifunctional linker are: N-succinimidyl-3-(2-pyridyldithio)-propionate (SPDP), N-succinimidyl-4-(2-pyridyldithio)butyrate (SPDB), N-succinimidyl-4-(2-pyridyl-dithio)pentanoate (SPP), N-succinimidyl-3-(2-pyridyldithio)-butyrate (SDPB), 2-iminothiolane, N-succinimidyl-4-(5-nitro-2-pyridyldithio) butyrate (SNPB), N-succinimidyl 4-(5-nitro-2-pyridyldithio)-pentanoate (SNPP), N-sulfosuccinimidyl-4-(5-nitro-2-pyridyldithio) butyrate (SSNPB), N-succinimidyl-4-methyl-4-(5-nitro-2-pyridyldithio)-pentanoate (SMNP), N-sulfosuccinimidyl 4-(5-nitro-2-pyridyldithio)-pentanoate (SSNPP), 4-succinimidyl-oxycarbonyl-α-methyl-α-(2-pyridyldithio)-toluene (SMPT), N-sulfosuccinimidyl-4-methyl-4-(5-nitro-2-pyridyldithio)pentanoate (SSMNP); N-succinimidyl-4-methyl-4-(2-pyridyldithio)pentanoate (SMPDP), N-succinimidyl-4-(5-N,N-dimethyl-carboxamido-2-pyridyldithio) butyrate (SCPB), N-sulfosuccinimidyl-4-(5-N,N-dimethyl-carboxamido-2-pyridyldithio) butyrate (SSCPB), N-succinimidyl-4,4-dimethyl-4-(2-pyridyl-dithio)pentanoate (SDMPDP), succinimidyl-4-(N-maleimidomethyl)cyclohexane-1-carboxylate (SMCC), N-succinimidyl-4-(iodoacetyl)-aminobenzoate (SIAB), bis-maleimidopolyethyleneglycol (BMPEG), BM(PEG)_{1˜20}, N—(β-maleimidopropyloxy)-succinimide ester (BMPS), iminothiolane (IT), dimethyl adipimidate HCl or derivatives of imidoesters, active esters (such as disuccinimidyl suberate), aldehydes (such as glutaraldehyde), bis-azido compounds (such as bis(p-azidobenzoyl) hexanediamine), bis-diazonium derivatives (such as bis-(p-diazonium-benzoyl)-ethylenediamine), diisocyanates (such as toluene 2,6-diisocyanate), and bis-active fluorine compounds (such as 1,5-difluoro-2,4-dinitrobenzene), gamma-maleimidobutyric acid N-succinimidyl ester (GMBS), E-maleimido-caproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproate) (a “long chain” analog of SMCC (LC-SMCC)), m-maleimido-benzoyl-N-hydroxysuccinimide ester (MBS), 4-(4-N-maleimidophenyl)-butyric acid hydrazide or HCl salt (MPBH), N-succinimidyl 3-(bromo-acetamido)propionate (SBAP), N-succinimidyl iodoacetate (SIA), kappa-maleimidoundecanoic acid N-succinimidyl ester (KMUA), N-succinimidyl 4-(p-maleimidophenyl)-butyrate (SMPB), succinimidyl-6-(beta-maleimido-propionamido)-hexanoate (SMPH), succinimidyl-(4-vinyl-sulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo-MBS), N-(gamma-maleimidobutryloxy)-sulfosuccinimdeester (sulfo-GMBS), N-(epsilon-maleimidocaproyloxy)-sulfo-succimido ester (sulfo-EMCS), N-(kappa-maleimidoundecanoyloxy)-sulfosuccinimide ester (sulfo-KMUS), and sulfosuccinimidyl 4-(p-maleimidophenyl)butyrate (sulfo-SMPB); or the commercially available linkers (such as from Thermo Scientific's Pierce: Imidoester Crosslinkers: DMA (Dimethyl adipimidate.2 HCl), DMP (Dimethyl pimelimidate.2 HCl), DMS (Dimethyl Suberimidate.2 HCl), DTBP (Dimethyl3,3′-dithiobispropionimidate.2 HCl); NHS-ester Crosslinkers-Amine Reactive: BS(PEG)₅ (Bis(succinimidyl) penta(ethylence glycol), BS(PEG)₉(Bis(succinimidyl) nona(ethylence glycol), BS³ (Bis[sulfosuccinimidyl] suberate), BSOCOES (Bis[2-(succinimidooxycarbonyloxy)-ethyl]sulfone), DSG (Disuccinimidyl glutarate), DSP (Dithiobis[succinimidyl propionate]), DSS (Disuccinimidyl suberate), DST (Disuccinimidyl tartarate), DTSSP (3,3′-Dithiobis[sulfo-succinimidylpropionate]), EGS (Ethylene glycol bis[succinimidylsuccinate]), Sulfo-EGS (Ethylene glycol bis[sulfo-succinimidylsuccinate]), TSAT (Tris-succinimidyl aminotriacetate), DFDNB (1,5-Difluoro-2,4-dinitrobenzene); Amine-to-Sulfhydryl Crosslinkers: Sulfo-SIAB (Sulfosuccinimidyl (4-iodoacetyl)aminobenzoate), SIAB (Succinimidyl (4-iodoacetyl)aminobenzoate), SBAP (Succinimidyl 3-(bromoacetamido)-propionate), SIA (Succinimidyl iodoacetate), Sulfo-SMCC (Sulfosuccinimidyl-4-(N-maleimidomethyl)-cyclohexane-1-carboxylate), SM(PEG)n (NHS-PEG-Maleimide Crosslinkers: Succinimidyl-([N-maleimidopropionamido])-#ethyleneglycol) ester, #=1 2, 3, 4, 5, 6, 7, 8, 9, 10, 11, 12, 13, 14, 15, 16, 17, 18, 19, 20, 21, 22, 23, 24), LC-SMCC (Succinimidyl 4-(N-maleimidomethyl) cyclohexane-1-carboxy-(6-amidocaproate)), Sulfo-EMCS (N-epsilon-Maleimidocaproyl-oxysulfosuccinimide ester), EMCS (N-epsilon-Malemidocaproyl-oxysuccinimide ester), Sulfo-GMBS (N-gamma-Maleimidobutyryl-oxysulfosuccinimide ester), GMBS (N-gamma-Maleimidobutyryl-oxysuccinimide ester), Sulfo-KMUS (N-kappa-maleimidoundecanoyl-oxysulfosuccinimide ester), Sulfo-MBS (m-maleimido-benzoyl-N-hydroxysulfosuccinimide ester), MBS (m-Maleimidobenzoyl-N-hydroxysuccinimide ester), Sulfo-SMPB ((Sulfo-succinimidyl 4-(p-maleiimidophenyl)butyrate), SMPB(Succinimidyl 4-(p-maleimido-phenyl)butyrate), AMAS N-(α-Maleimidoacetoxy) succinimide ester), BMPS (N-beta-Maleimidopropyloxy-succinimide ester), SMPH (Succinimidyl 6-[(beta-maleimido-propionamido)hexanoate]), PEG12-SPDP (2-Pyridyldithiol-tetraoxaoctatriacontane-N-hydroxysuccinimide), PEG4-SPDP (2-Pyridyldithiol-tetraoxatetradecane-N-hydroxysuccinimide), Sulfo-LC-SPDP (Sulfosuccinimidyl 6-[3′-(2-pyridyldithio)propionamido]-hexanoate), LC-SPDP (Succinimidyl 6-[3-(2-pyridyldithio)-propionamido]hexanoate), SMPT (4-Succinimidyl-oxycarbonyl-alpha-methyl-alpha(2-pyridyldithio)toluene); Carboxyl-to-Amine Crosslinkers: DCC (Dicyclohexylcarbodiimide), EDC (1-Ethyl-3-(3-dimethylaminopropyl) carbodiimide); Photoreactive Crosslinkers: ANB-NOS (N-5-Azido-2-nitrobenzoyloxy-succinimide), NHS-Diazirine (SDA) Crosslinkers: SDA (NHS-Diazirine)(Succinimidyl4,4′-azipentanoate), LC-SDA (NHS-LC-Diazirine) (Succinimidyl 6-(4,4′-azipentanamido)-hexanoate), SDAD (NHS-SS-Diazirine) (Succinimidyl 2-([4,4′-azipentanamido]ethyl)-1,3′-dithiopropionate), Sulfo-SDA (Sulfo-NHS-Diazirine) (Sulfosuccinimidyl 4,4′-azipentanoate), Sulfo-LC-SDA (Sulfo-NHS-LC-Diazirine) (Sulfosuccinimidyl 6-(4,4′-azipentanamido)-hexanoate), Sulfo-SDAD (Sulfo-NHS-SS-Diazirine)(Sulfosuccinimidyl 2-([4,4′-azipentanamido]ethyl)-1,3′-dithiopropionate), Sulfo-SANPAH (Sulfosuccinimidyl 6-(4′-azido-2′-nitrophenylamino)-hexanoate), SPB (Succinimidyl-[4-(psoralen-8-yloxy)]-butyrate); Sulfhydryl-to-Carbohydrate Crosslinkers: BMPH (N-beta-Maleimidopropionic acid hydrazide-TFA), EMCH (N-epsilon-Maleimidocaproic acid hydrazide-TFA), KMUH (N-kappa-maleimidoundecanoic acid hydrazide-TFA), MPBH (4-(4-N-Maleimidophenyl)butyric acid hydrazide-HCl), PDPH (3-(2-Pyridyldithio)propionyl hydrazide); Sulfhydryl-to-Hydroxyl Crosslinkers: PMPI (p-Maleimidophenyl isocyanate); Sulfhydryl-to-Sulfhydryl Crosslinkers: BM(PEG)2 (1,8-Bismaleimido-diethyleneglycol), BM(PEG)3 (1,11-Bismaleimidotriethyleneglycol), BMB (1,4-Bismaleimidobutane), BMDB (1,4-Bismaleimidyl-2,3-dihydroxybutane), BMH (Bismaleimido-hexane), BMOE (Bismaleimidoethane), DTME (Dithiobismaleimido-ethane), TMEA (Tris(2-maleimidoethyl)amine) and SVSB (succinimidyl-(4-vinylsulfone)benzoate).

The bis-maleimide or bis-2-pyridyldithiol reagents allow the attachment of the thiol group of a thiol-containing cell binding agent (such as antibody) to a thiol-containing drug moiety, label, or linker intermediate, in a sequential or concurrent fashion. Other functional groups besides maleimide and pyridyldithiol, which are reactive with a thiol group of a cell binding agent, drug moiety, label, or linker intermediate include iodoacetamide, bromoacetamide, vinyl pyridine, disulfide, pyridyl disulfide, isocyanate, and isothiocyanate.

In additional embodiments, the linker may be composed of one or more linker components. The exemplary linker components are:

1. The self-immolative linker components:

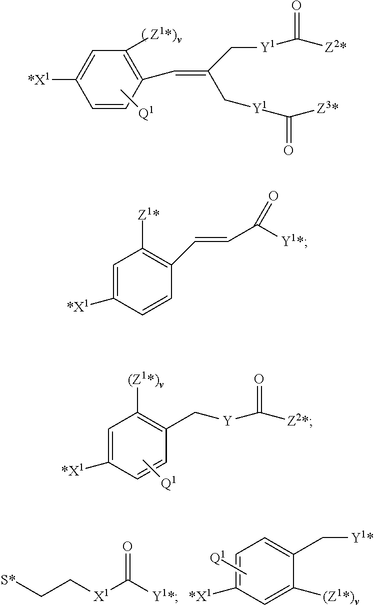

wherein the (*) atom is the point of attachment of additional spacer or releasable linker units, or the cytotoxic agent, and/or the binding molecule (CBA); X¹, Y¹, Z² and Z³ are independently NH, or O, or S; Z¹ is H, or NH, or O or S independently. v is O or 1; Q¹ is independently H, OH, C₁˜C₆ alkyl, (OCH₂CH₂)ₙ F, Cl, Br, I, OR₁, or SR₁, NR₁R₂, N═NR₁, N═R₁, NR₁R₂, NO₂, SOR₁R₂, SO₂R₁, SO₃R₁, OSO₃R₁, PR₁R₂, POR₁R₂, PO₂R₁R₂, OPO(OR₁)(OR₂), or OCH₂PO(OR₁(OR₂) wherein R₁ and R₂ are described above, preferably independently selected from H, C₁˜C₈ of alkyl; C₂˜C₈ of alkenyl, alkynyl, heteroalkyl; C₃˜C₈ of aryl, heterocyclic, carbocyclic, cycloalkyl, heterocycloalkyl, heteroaralkyl, alkylcarbonyl; or pharmaceutical cation salts

2. The examples of non-self-immolative linker components:

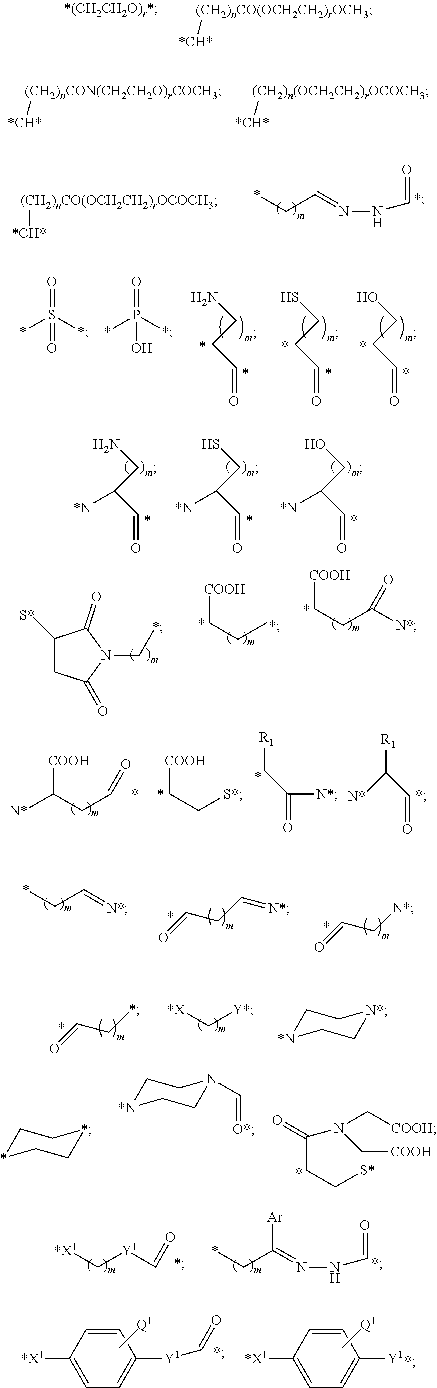

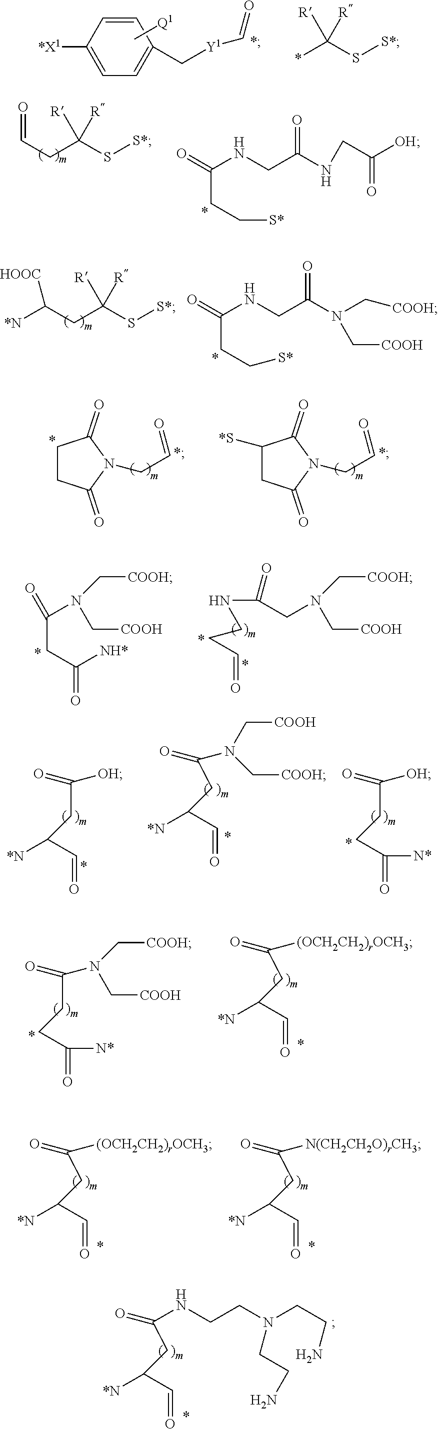

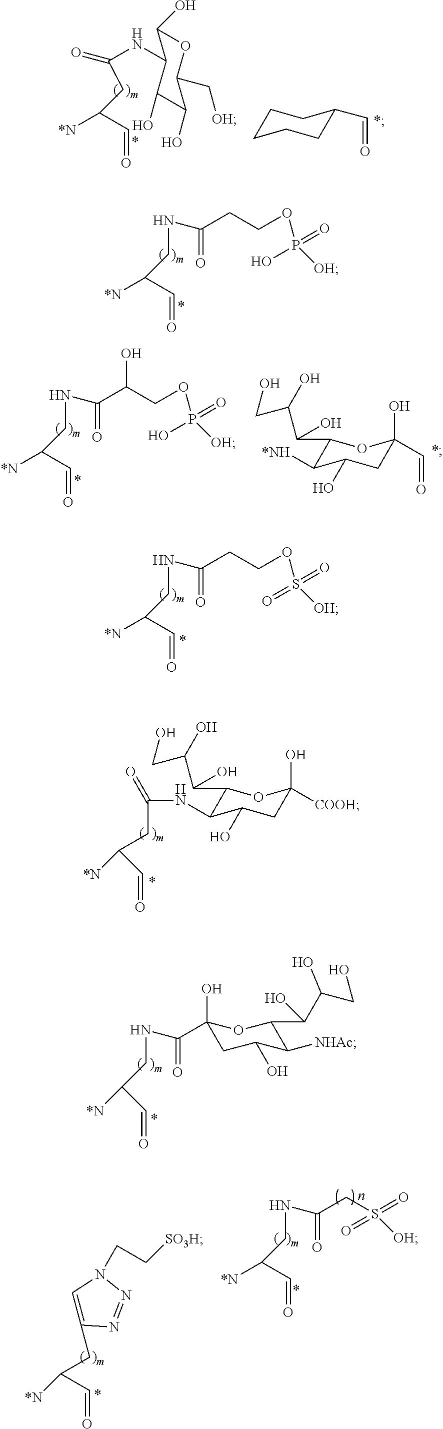

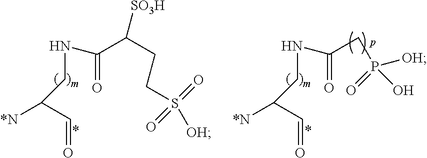

wherein the (*) atom is the point of attachment of additional spacer or releaseable linkers, the cytotoxic agents, and/or the binding molecules; X¹, Y¹, Q¹, R1, R′, R″ are defined above; r is 1˜20, m and n are 1˜6.

3. Exemplary linker components may include 6-maleimidocaproyl (“MC”), maleimidopropanoyl (“MP”), valine-citrulline (“val-cit” or “vc”), alanine-phenylalanine (“ala-phe” or “af”), p-aminobenzyloxy-carbonyl (“PAB”), N-succinimidyl 4-(2-pyridylthio)pentanoate (“SPP”), N-succinimidyl 4-(N-maleimidomethyl)cyclohexane-1 carboxylate (“SMCC”), N-Succinimidyl (4-iodo-acetyl)amino-benzoate (“SIAB”), ethyleneoxy (—CH₂CH₂O—) as one or more repeating units (“EO” or “PEO”). Additional linker components are known in the art and some are described through this patent application.

In additional embodiments, the linker may comprise amino acid residues. Exemplary amino acid linker components include a dipeptide, a tripeptide, a tetrapeptide or a pentapeptide. Exemplary dipeptides include: valine-citrulline (VC or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline. Amino acid linker components can be designed and optimized in their selectivity for enzymatic cleavage by particular enzymes, for example, a tumor-associated protease, cathepsin B, C and D, or a plasmin protease.

In the cell-binding agent-drug conjugates of the invention, cell-binding agent (Q) is conjugated to one or more drug moieties (Drug, or PBD derivatives), e.g. about 1 to about 20 drug moieties per cell-binding agent, through a bifunctional linker (L). The conjugate of Formula (I), (II), (III), and (IV) may be prepared by several routes, employing organic chemistry reactions, conditions, and reagents known to those skilled in the art, including: (1) reaction of a nucleophilic group of a cell-binding agent with a bivalent linker reagent, to form Q-L, via a covalent bond, followed by reaction with a drug moiety Drug; and (2) reaction of a nucleophilic group of a drug moiety with a bivalent linker reagent, to form Drug-L, via a covalent bond, followed by reaction with the nucleophilic group of a cell-binding agent.

To synthesize the conjugates of Formula (I), (II), (III) or/and (IV) in general, a function group E₃ or/and E₃′ on Formula (V), (VI), (VII) and (VIII) reacts one, two or more residues of a cell binding molecule at 0-60° C., pH 5˜9.5 aqueous media with or without addition of 0˜30% of water mixable (miscible) organic solvents, such as DMA, DMF, ethanol, methanol, acetone, acetonitrile, THF, isopropanol, dioxane, propylene glycol, or ethylene diol, following by dialysis or chromatographic purification to form a conjugate compound of Formula (I), (II), (III) or/and (IV). Some of the residue (reacting group for conjugation) of the cell-binding molecule can be obtained through protein engineering.

The thiol or amine groups on a cell-binding agents, such as an antibody, are nucleophilic and capable of reacting to form covalent bonds with electrophilic groups on linker reagents and drug-linker intermediates including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents.

Nucleophilic groups on antibodies or proteins can react to electrophilic groups on a function linker following by reaction with a cytotoxic agent, or directly react to a linker-cytotoxic agent moiety to form covalent bond conjugate of a cell binding agent-cytotoxic agent. Nucleophilic groups on antibodies or proteins include, but are not limited to: (i)N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker-cytotoxic agent moieties including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges which may be made reactive by treatment with a reducing agent such as DTT (dithiothreitol) or tricarbonylethyl-phosphine (TCEP) (Getz et al (1999) Anal. Biochem. Vol 273:73-80; Soltec Ventures, Beverly, Mass.), dithioerythritol (DTE), L-glutathione (GSH), 2-mercaptoethylamine (β-MEA), or/and beta mercaptoethanol (β-ME, 2-ME). Each cysteine bridge will thus form, theoretically, two reactive thiol nucleophiles. Alternatively, sulfhydryl groups can be introduced into antibodies through modification of lysine residues, e.g., by reacting lysine residues with 2-iminothiolane (Traut's reagent), resulting in conversion of an amine into a thiol. Reactive thiol groups may be introduced into an antibody by introducing one, two, three, four, or more cysteine residues (e.g., by preparing variant antibodies comprising one or more non-native cysteine amino acid residues). Thus free thiol on the cell binding agents can be conjugated to the thiol-reactive groups, such as, a maleimide, an iodoacetamide, a pyridyl disulfide, or other thiol-reactive groups on the cytotoxic agents, or linker-cytotoxic agent intermediates of the invention. Some unconjugated free thiols on the antibodies can be reoxidized to reform interchain and intrachain disulfide bonds.

Antibody-drug conjugates of the invention may also be produced by reaction between an electrophilic group on an antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an antibody is modified to introduce electrophilic moieties that are capable of reacting with nucleophilic substituents on the linker reagent or drug. In another embodiment, the sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody that can react with appropriate groups on the drug (Hermanson, Bioconjugate Techniques). In another embodiment, antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Examples of these kinds of two-step conjugations are depicted below:

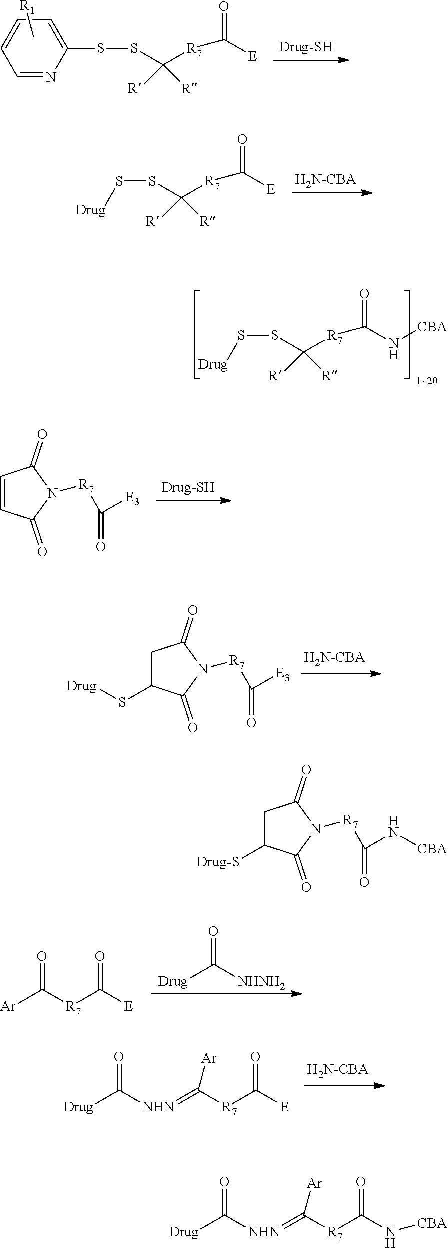

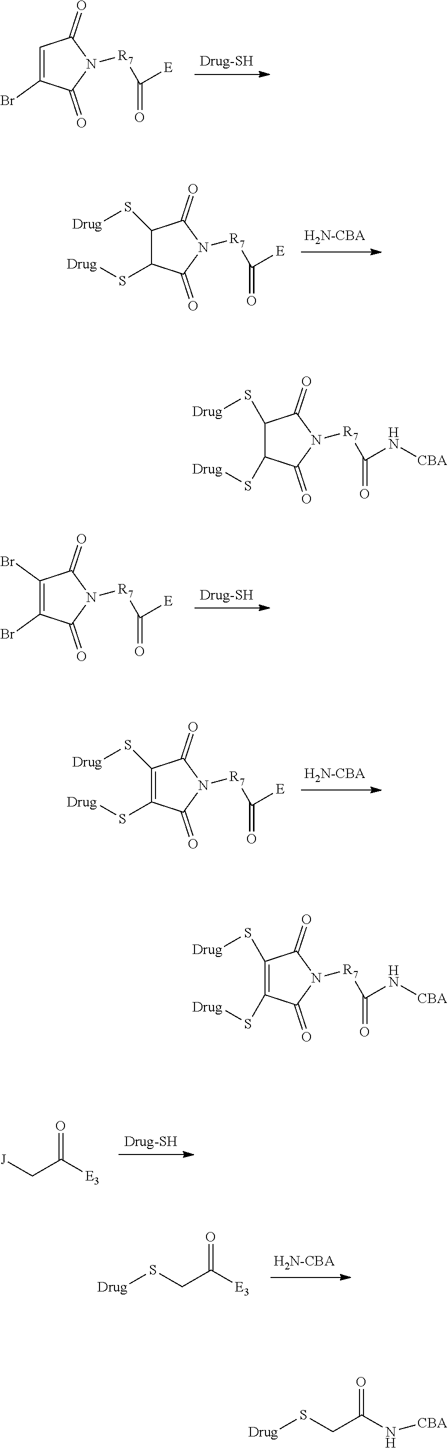

wherein E includes, but is not limited to, such as hydroxysuccinimidyl esters (NHS, Sulfo-NHS, etc), 4-nitrophenyl esters, pentafluorophenyl esters, tetrafluorophenyl (includes sulfo-tetrafluorophenyl) esters, anhydrides, acid chlorides, sulfonyl chlorides, isocyanates and isothiocyanates. R′ and R″ are independently H or CH₃, or C₂H₅; J is F, Cl, Br, I, tosylate (TsO), mesylate (MsO), nitrophenol, dinitrophenol, or pentaflourophenol.

It is to be understood that where more than one nucleophilic group on the cell binding agents, such as an antibody, reacts with a drug-linker intermediate or linker reagent followed by drug moiety reagent, then the resulting product is a mixture of the cell binding agent-cytoxic agent conjugates with a distribution of one or more drug moieties attached to an antibody. The average number of drugs per antibody may be calculated from the mixture by a dual ELISA antibody assay, which is specific for antibody and specific for the drug. Individual conjugate molecules may be identified in the mixture by mass spectroscopy and separated by HPLC, e.g. hydrophobic interaction chromatography. In certain embodiments, a homogeneous conjugate with a single loading value may be isolated from the conjugation mixture by electrophoresis or chromatography.

In the conjugation, The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of lysine or cysteine residues is modified for control of the number and/or position of linker-drug attachments (such as thioMab or thioFab).

The synthetic conjugate may be purified by standard biochemical means, such as gel filtration on a Sephadex G25 or Sephacryl S300 column, adsorption chromatography, and ion exchange or by dialysis. In some cases, a small molecule as a cell-binding agent (e.g. folic acid, melanocyte stimulating hormone, EGF etc) conjugated with a small molecular drugs can be purified by chromatography such as by HPLC, medium pressure column chromatography or ion exchange chromatography.

The aqueous solutions for the modification of cell-binding agents are buffered between pH 4 and 9, preferably between 6.0 and 7.5 and can contain any non-nucleophilic buffer salts useful for these pH ranges. Typical buffers include phosphate, acetate, triethanolamine HCl, HEPES, and MOPS buffers, which can contain additional components, such as cyclodextrins, hydroxypropyl-β-cyclodextrin, polyethylene glycols, sucrose and salts, for examples, NaCl and KCl. After the addition of the drug-linker of Formula (V), (VI), (VII) or Formula (VIII) into the solution containing the reduced cell-binding molecules, the reaction mixture is incubated at a temperature of from 4° C. to 55° C., preferably at 15° C.—ambient temperature. The progress of the reaction can be monitored by measuring the decrease in the absorption at a certain UV wavelength, such as at 252 nm, or increase in the absorption at a certain UV wavelength, such as 280 nm, or the other appropriate wavelength. After the reaction is complete, isolation of the modified cell-binding agent can be performed in a routine way, using for example a gel filtration chromatography, an ion exchange chromatography, an adsorptive chromatography or column chromatography over silica gel or alumina, crystallization, preparatory thin layer chromatography, ion exchange chromatography, or HPLC.

The extent of modification can be assessed by measuring the absorbance of the nitropyridine thione, dinitropyridine dithione, pyridine thione, carboxylamidopyridine dithione and dicarboxyl-amidopyridine dithione group released via UV spectra. For the conjugation without a chromophore group, the modification or conjugation reaction can be monitored by LC-MS, preferably by HPLC-MS/MS, UPLC-QTOF mass spectrometry, or Capilary electrophoresis-mass spectrometry (CE-MS). The side chain cross-linkers described herein have diverse functional groups that can react with any cell-binding molecules, particularly a modified cell-binding molecule that possess a suitable substituent. For examples, the modified cell-binding molecules bearing an amino or hydroxyl substituent can react with drugs bearing an N-hydroxysuccinimide (NHS) ester, the modified cell-binding molecules bearing a thiol substituent can react with drugs bearing a maleimido or haloacetyl group. Additionally, the modified cell-binding molecules bearing a carbonyl (ketone or aldehyde) substituent either through protein engineering, enzymatical reaction or chemical modification can react with drugs bearing a hydrazide or an alkoxyamine. One skilled in the art can readily determine which modified drug-linker to be used based on the known reactivity of the available functional group on the modified cell-binding molecules.

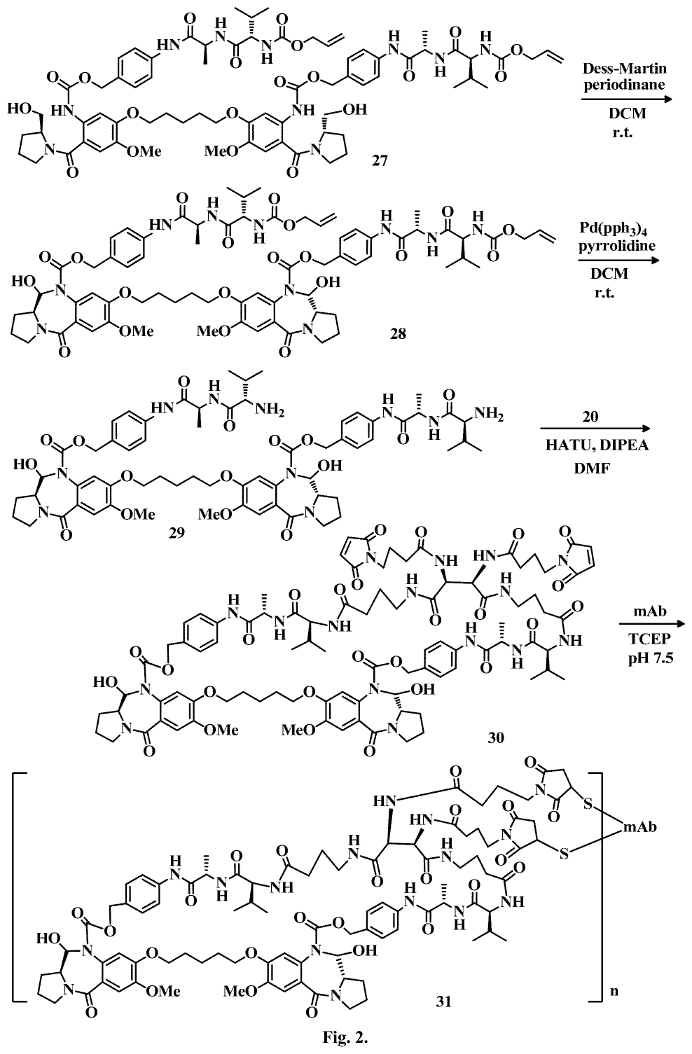

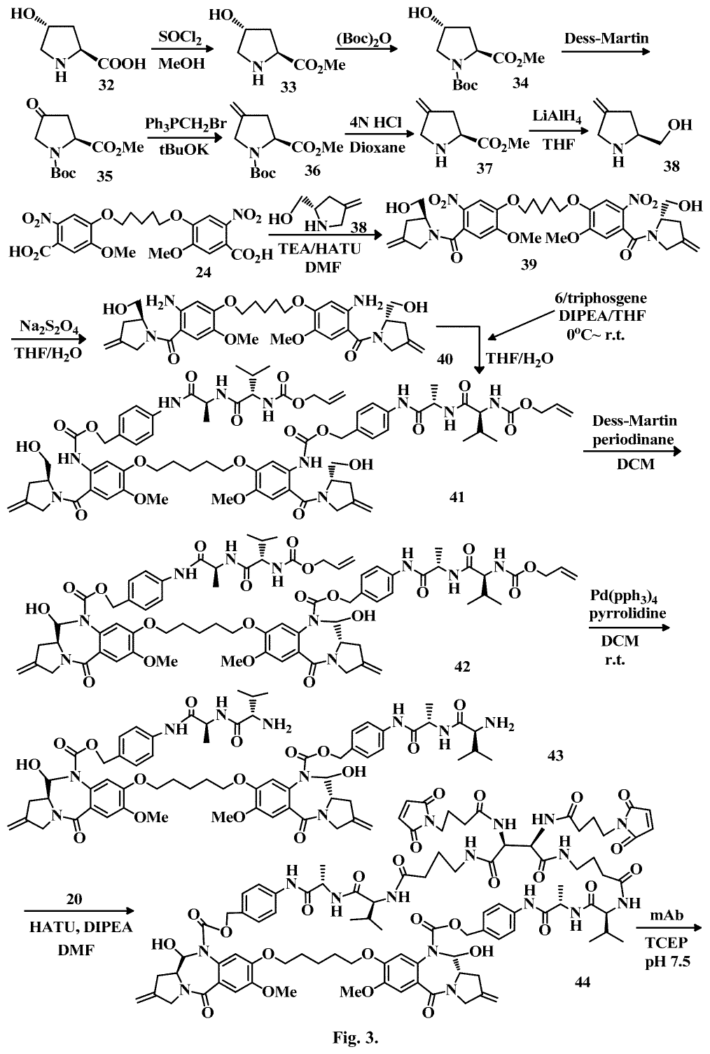

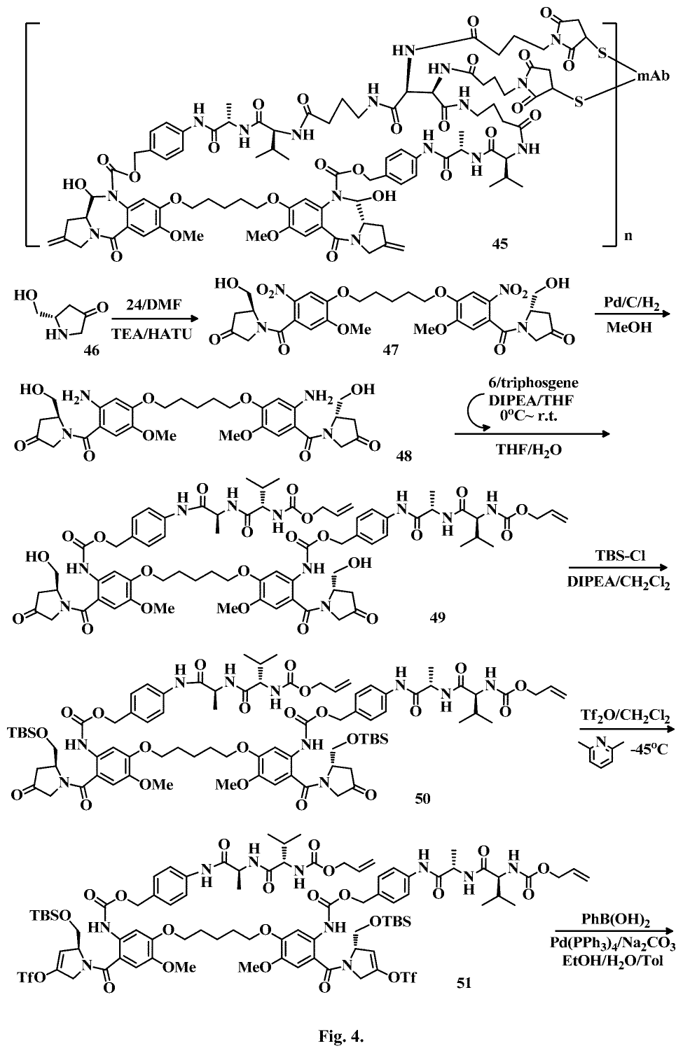

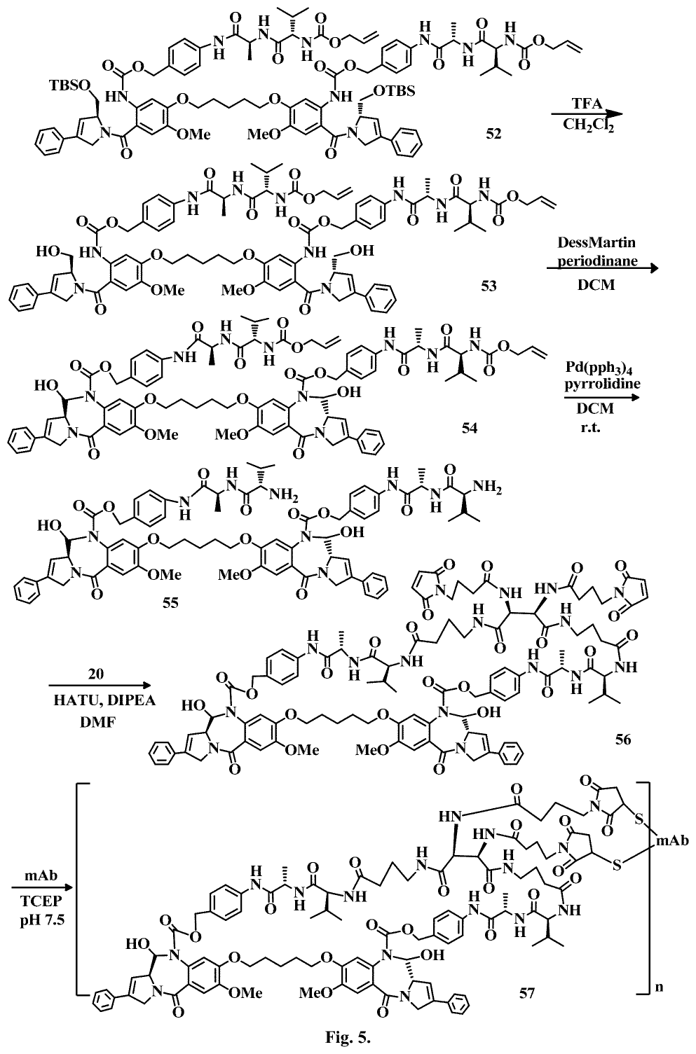

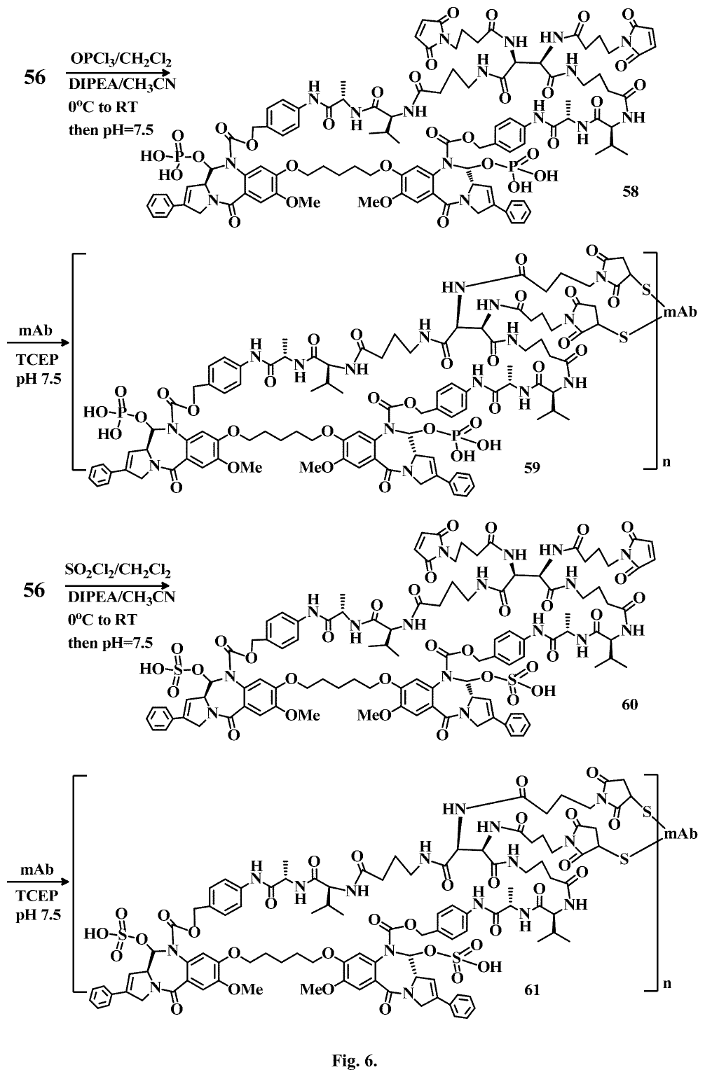

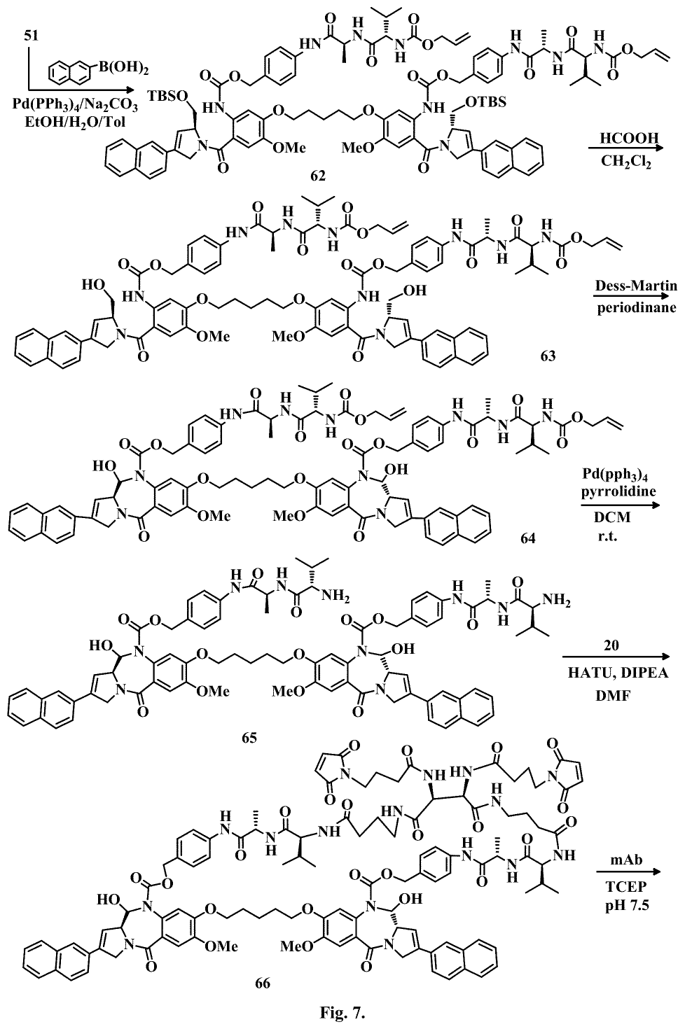

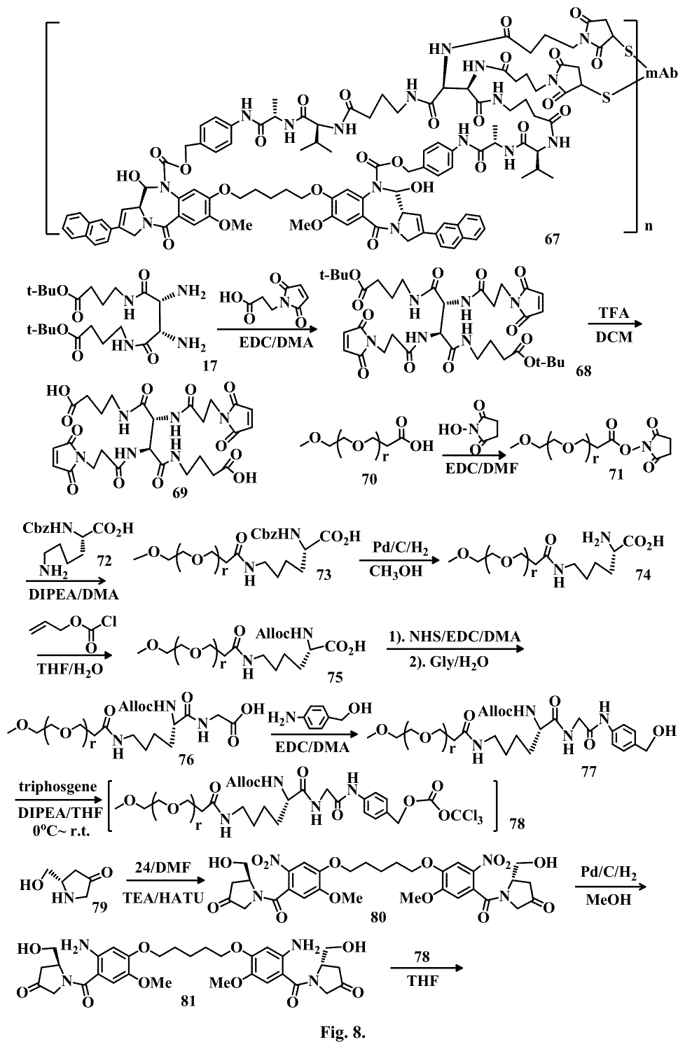

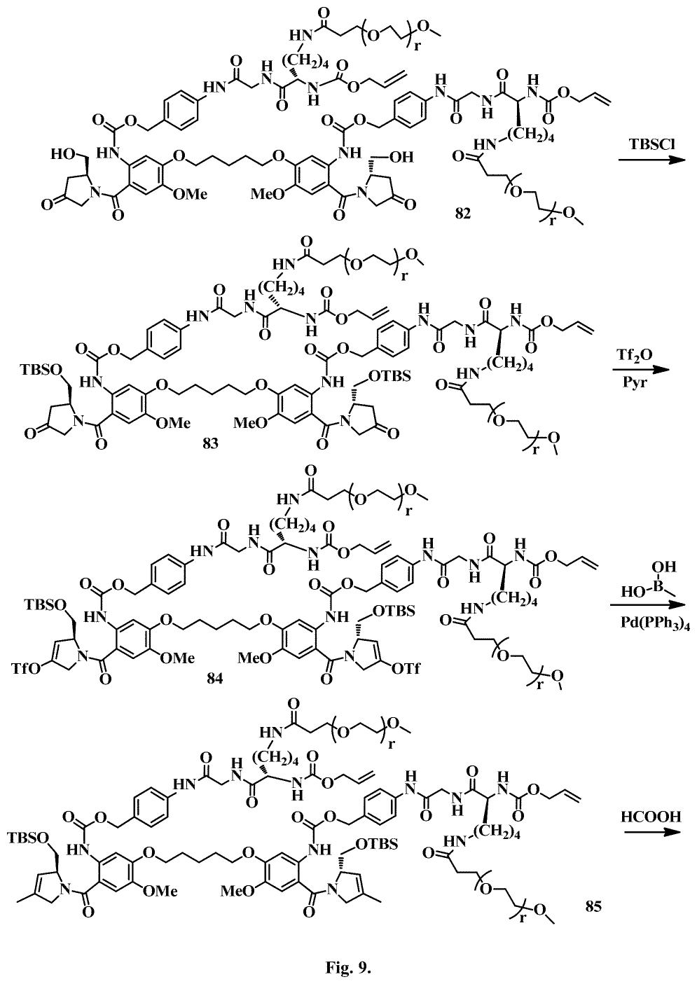

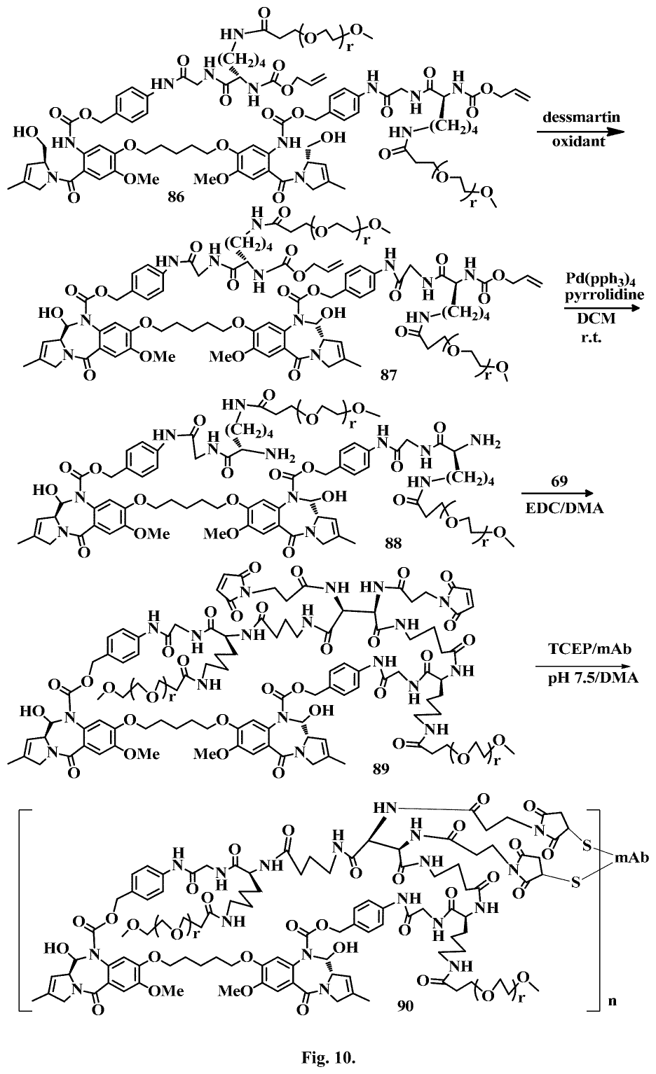

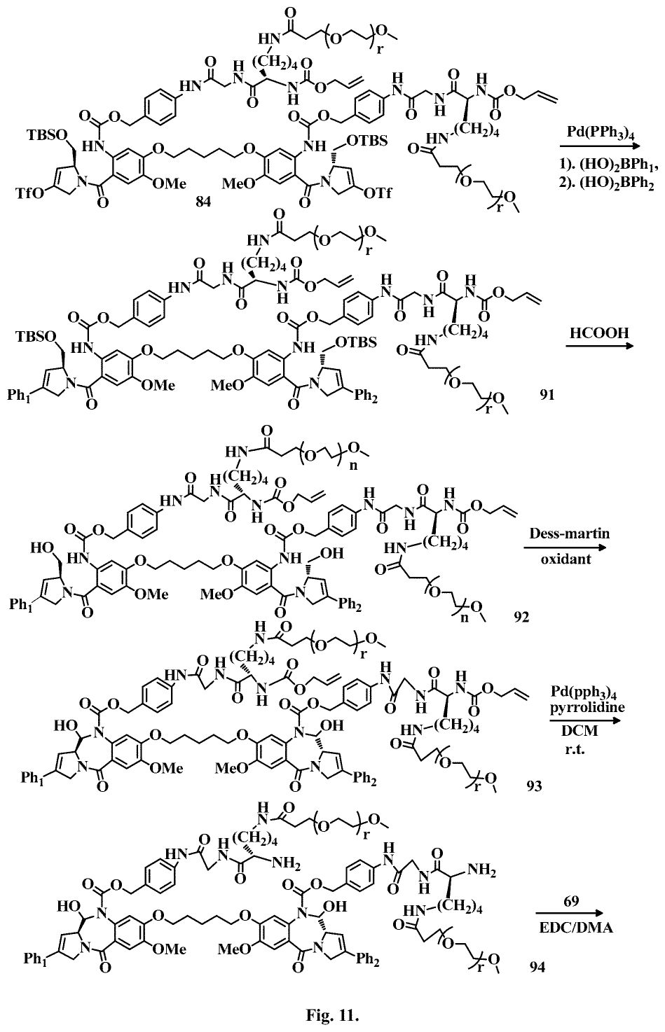

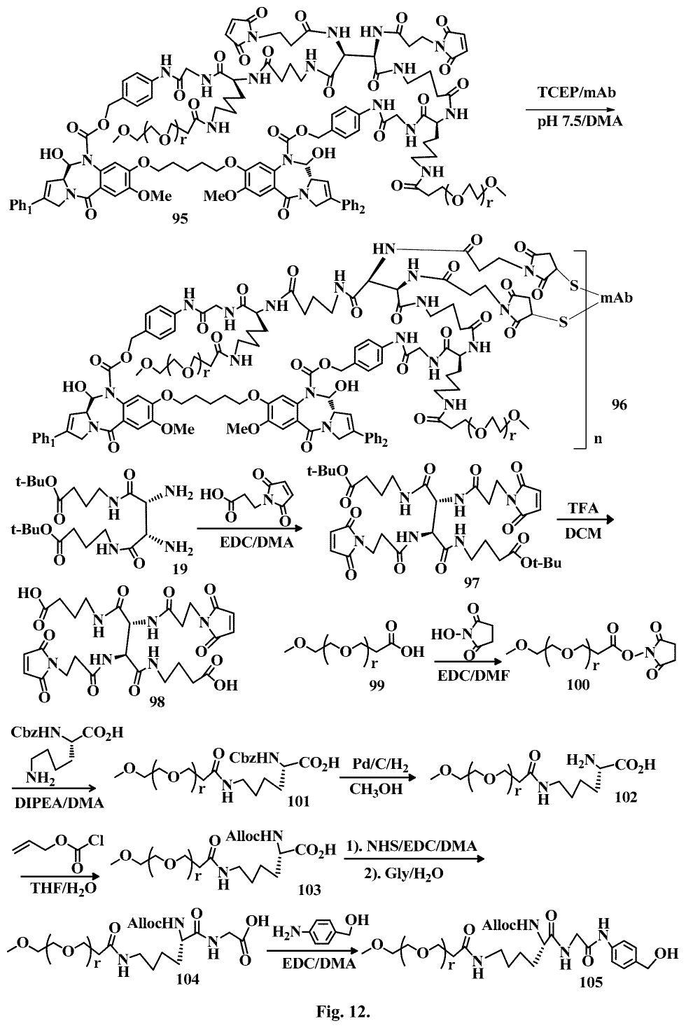

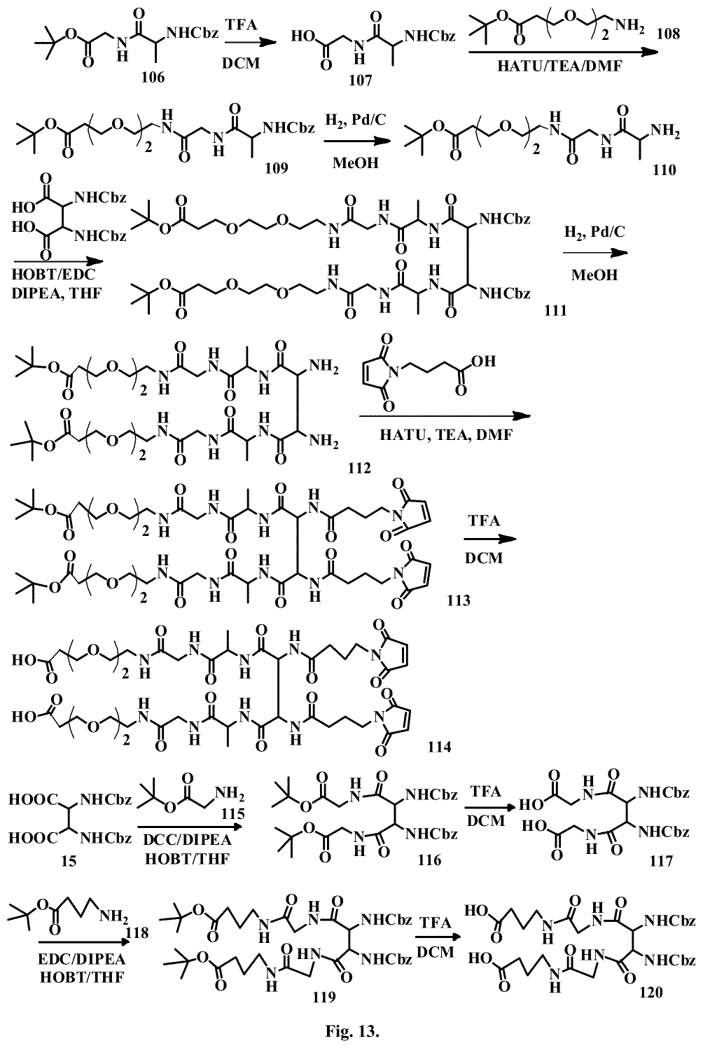

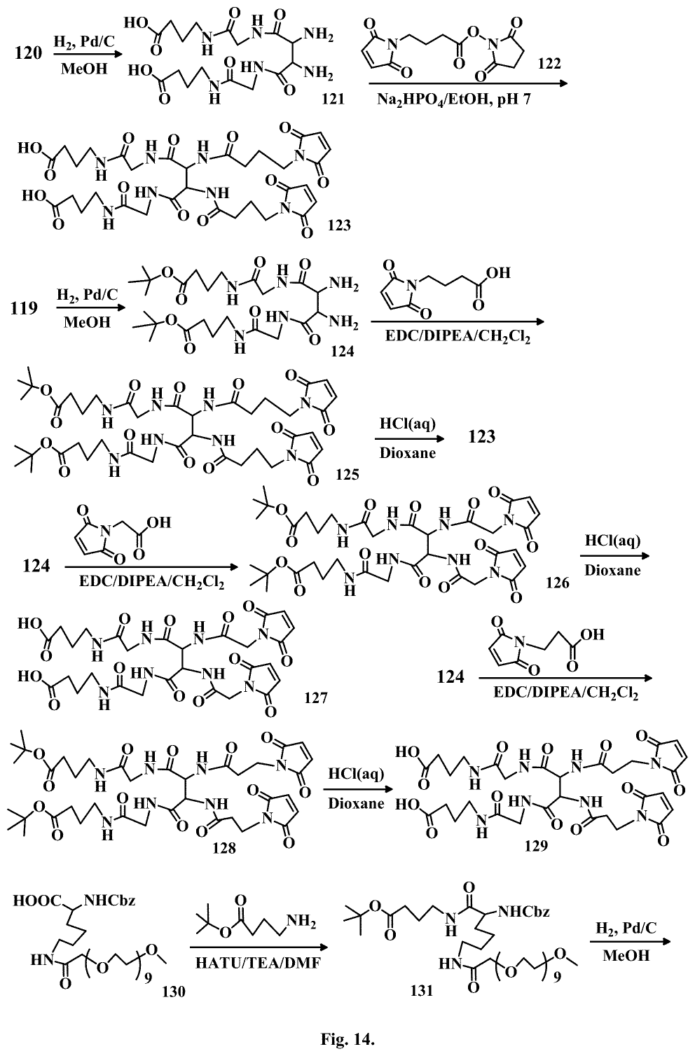

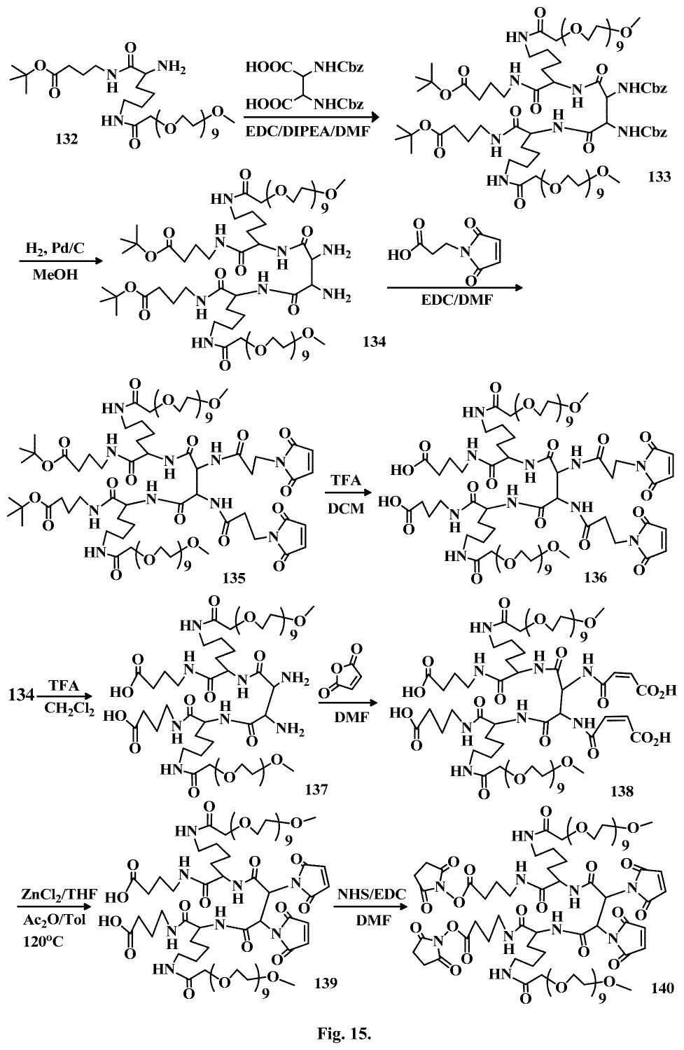

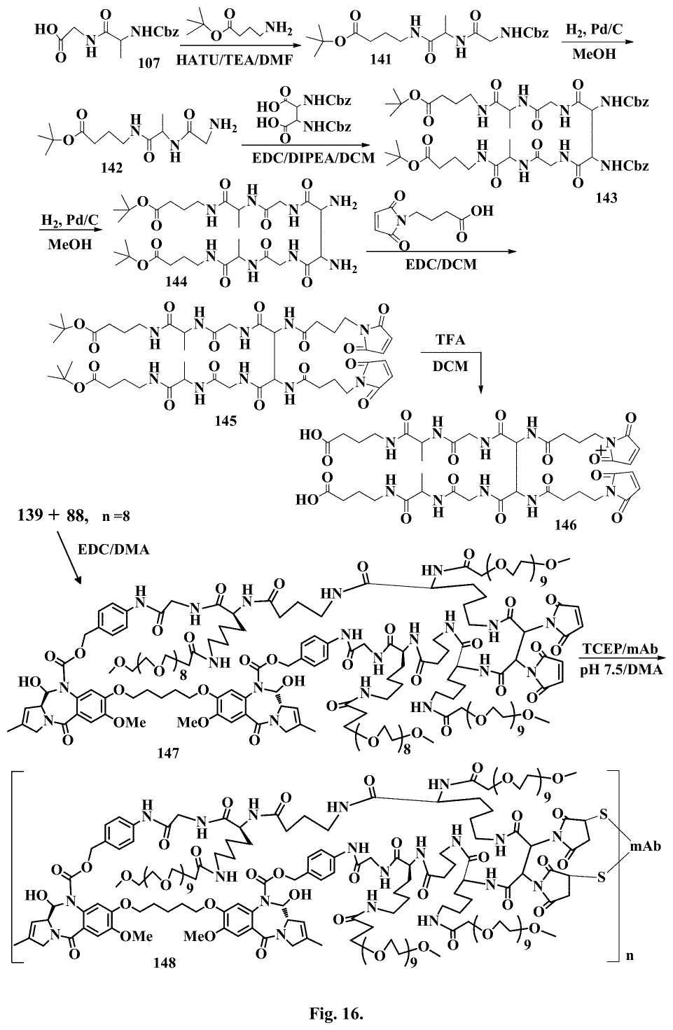

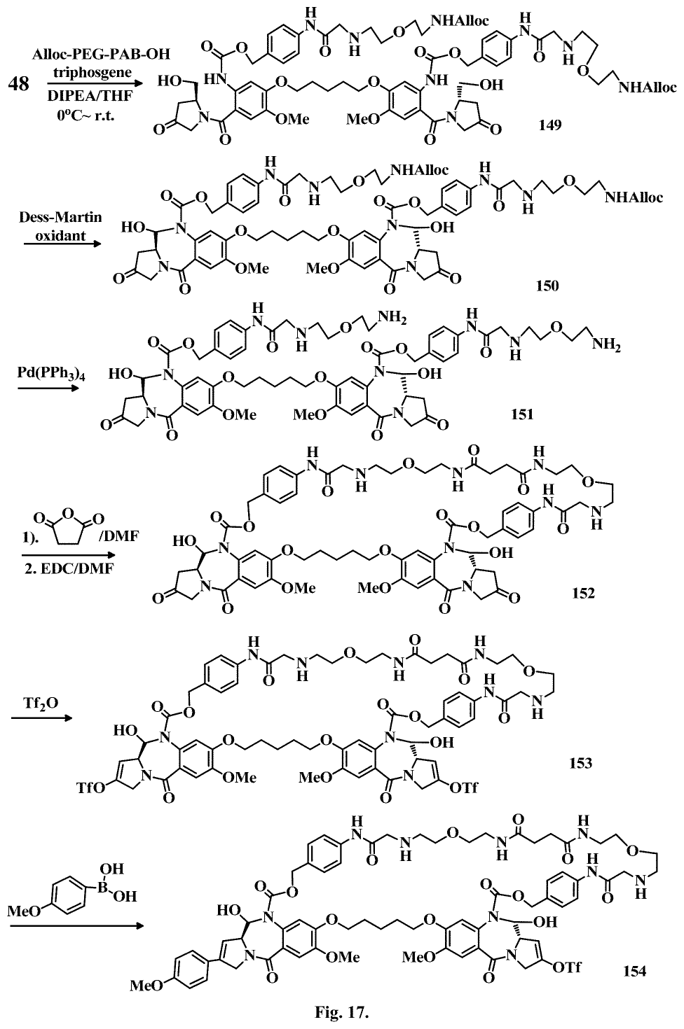

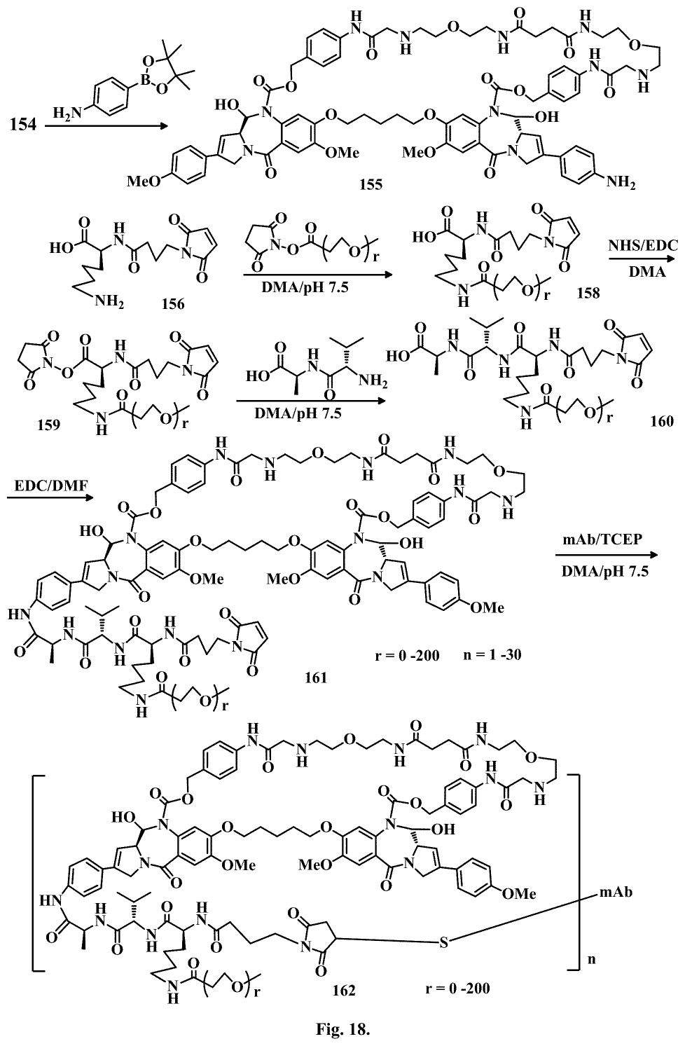

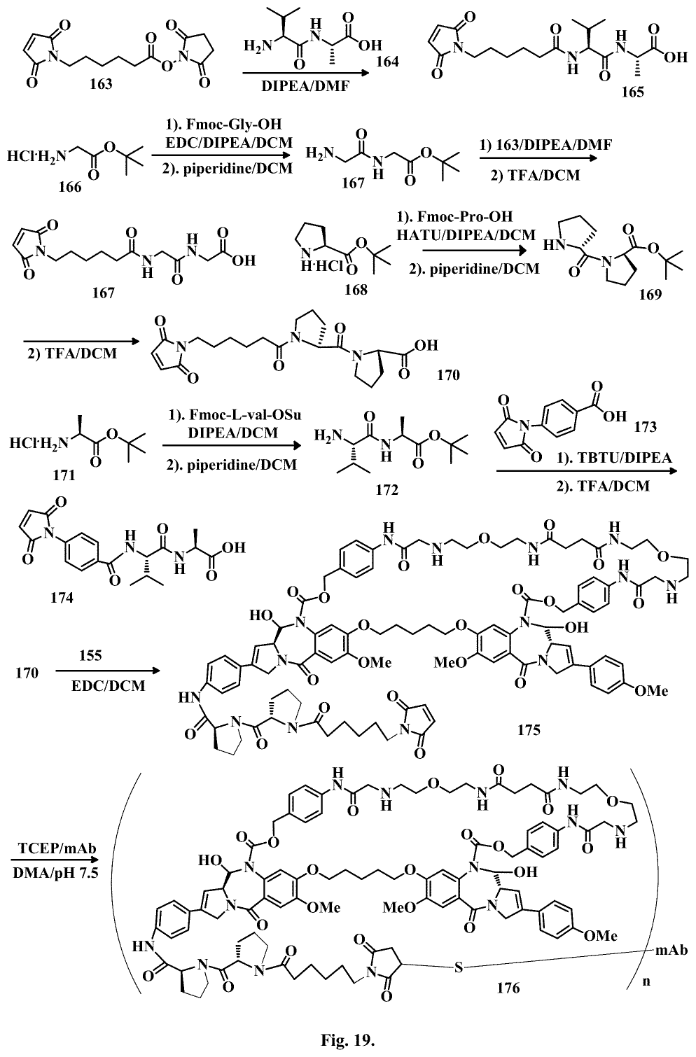

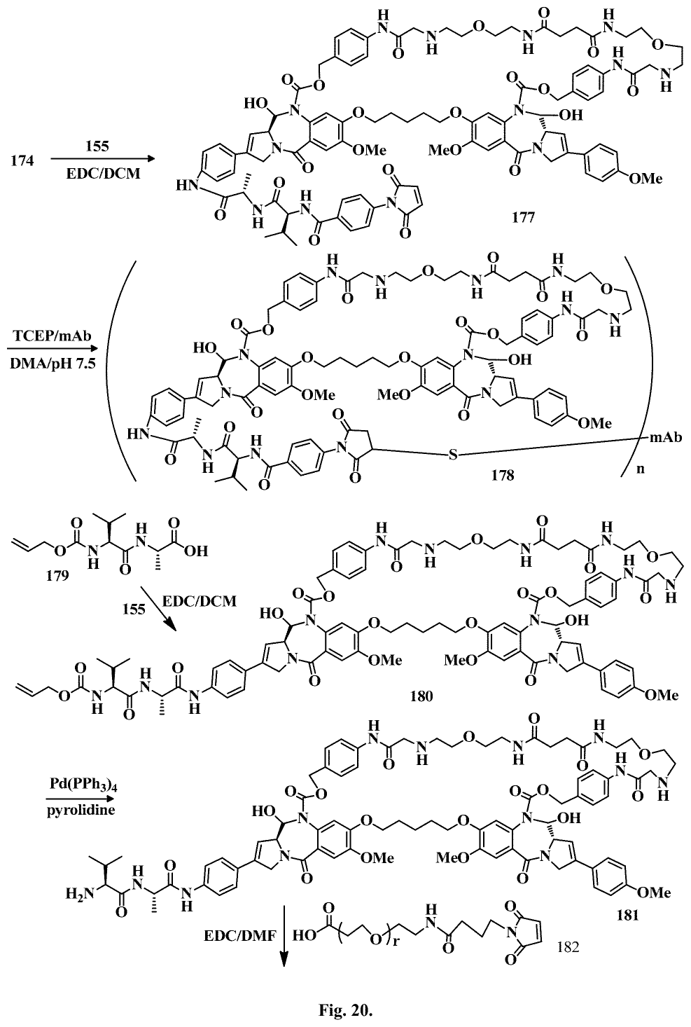

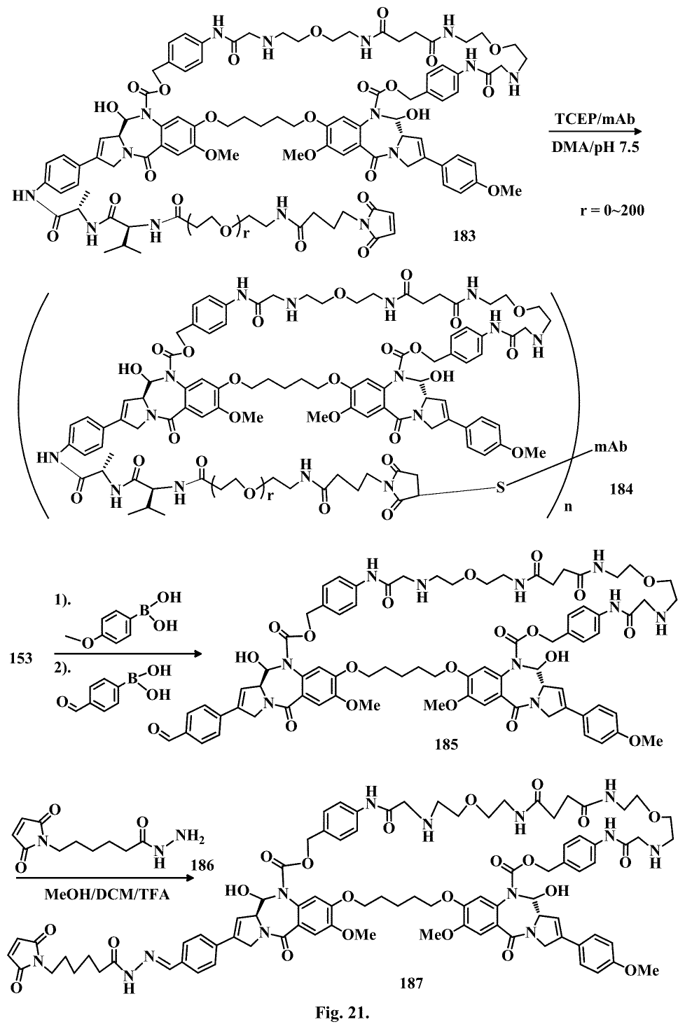

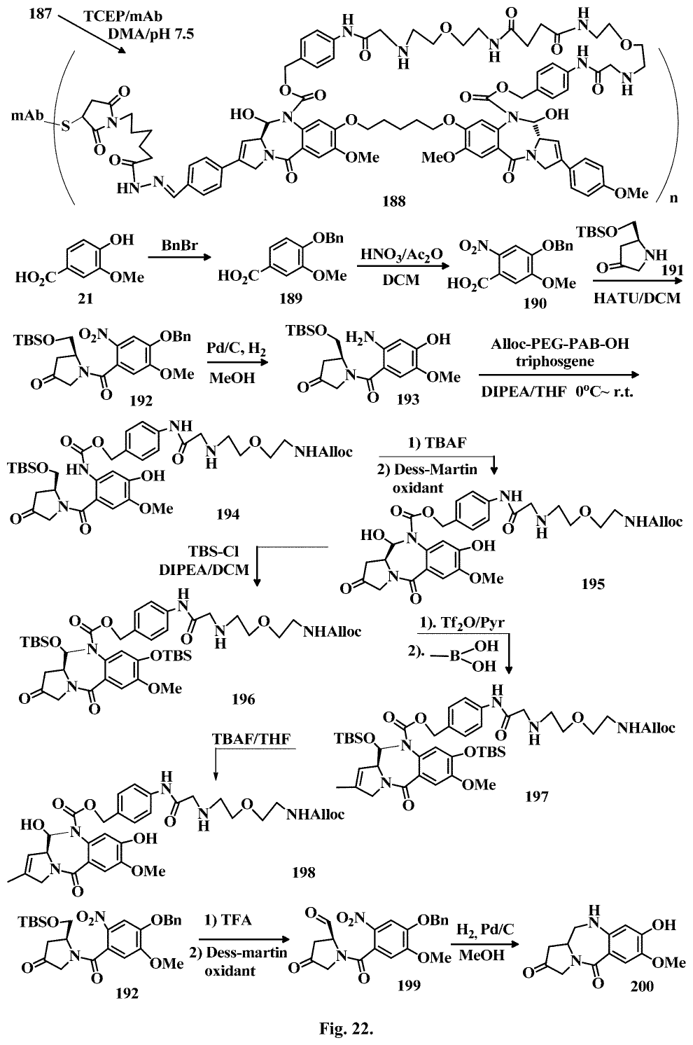

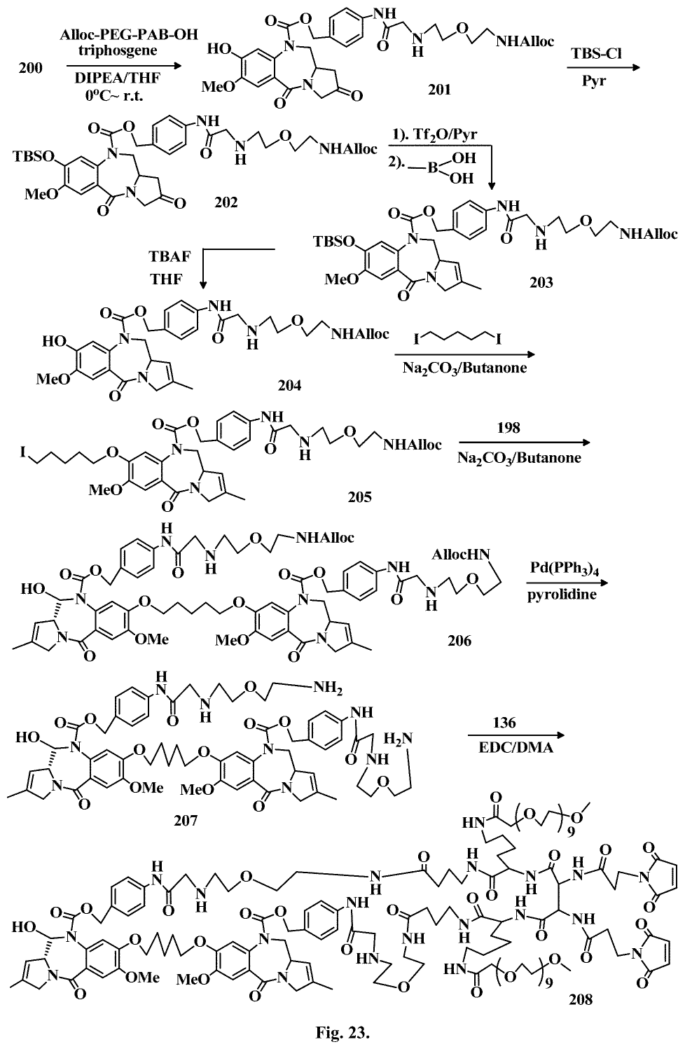

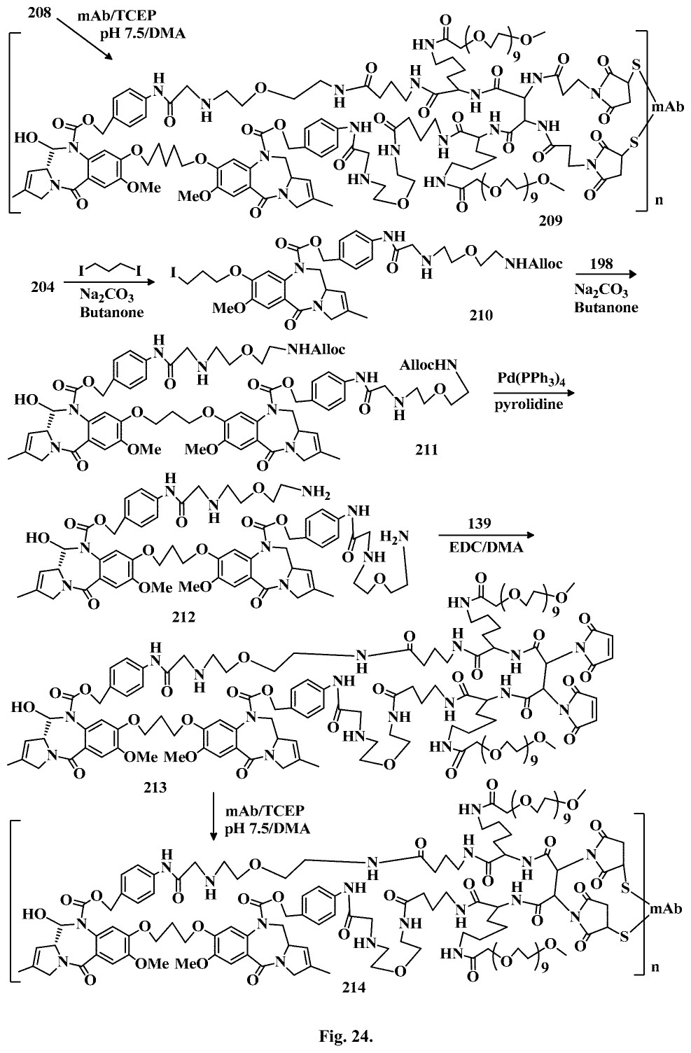

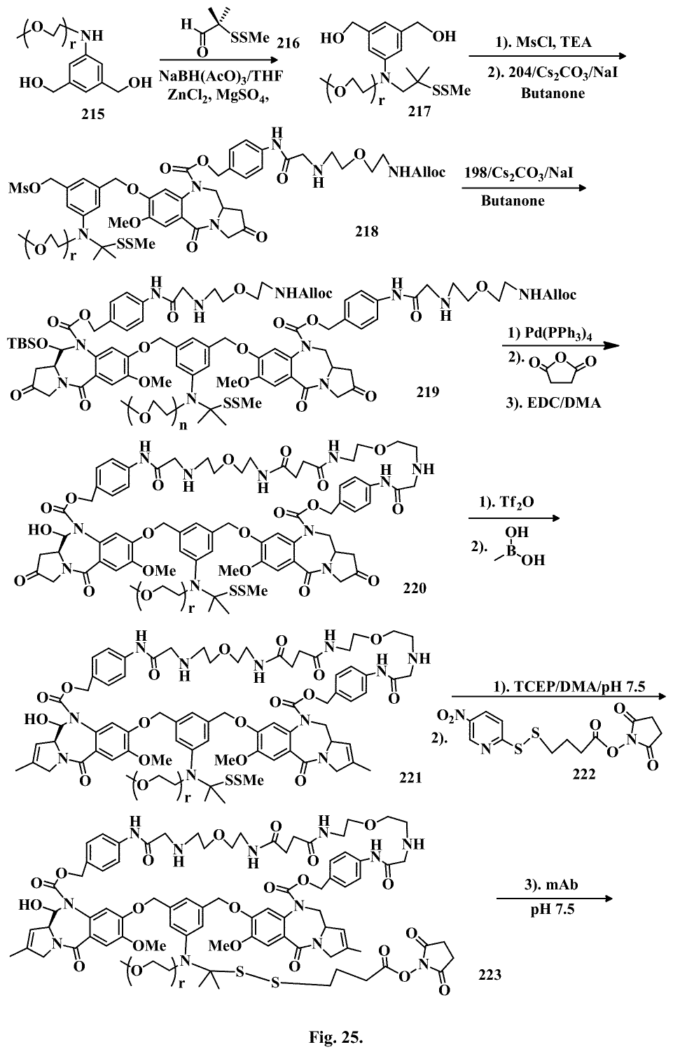

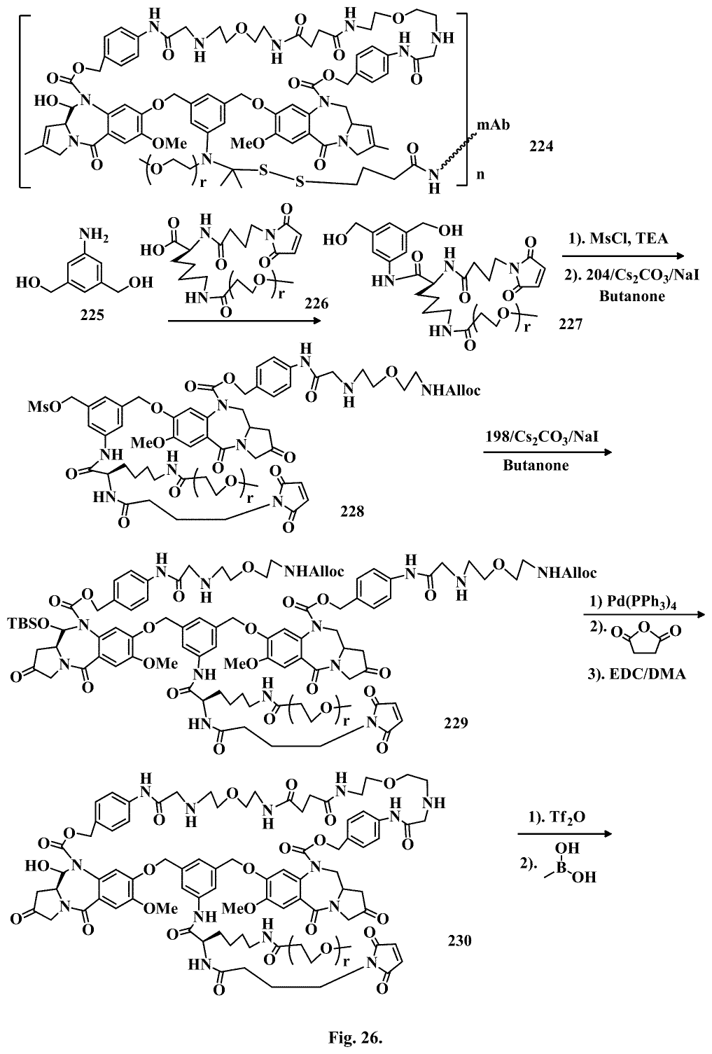

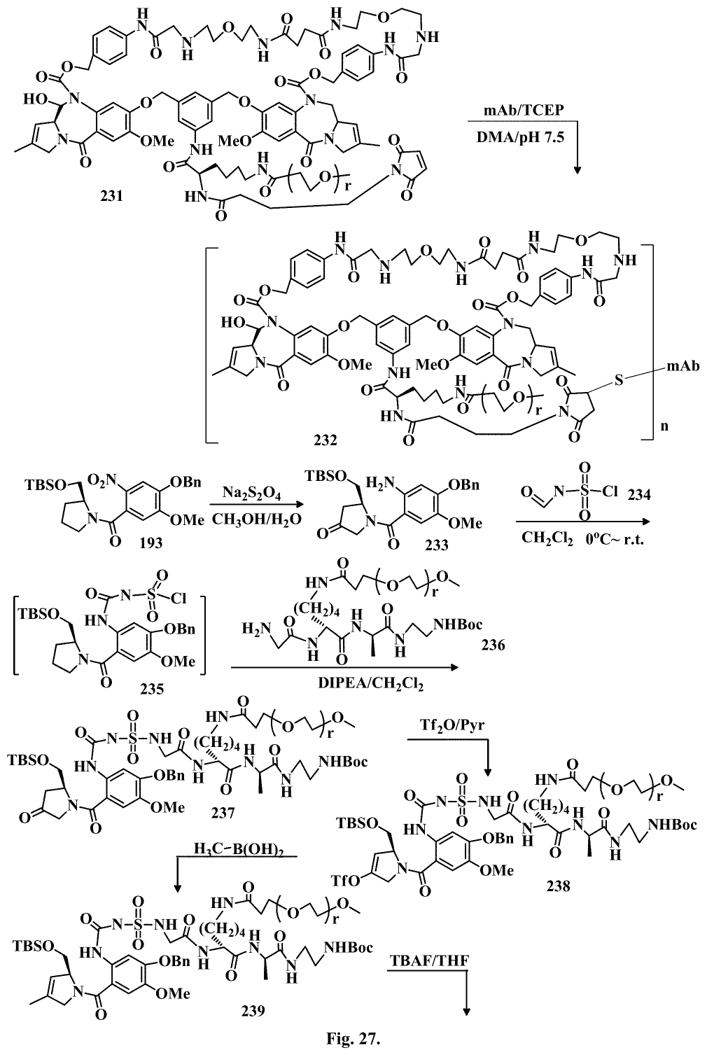

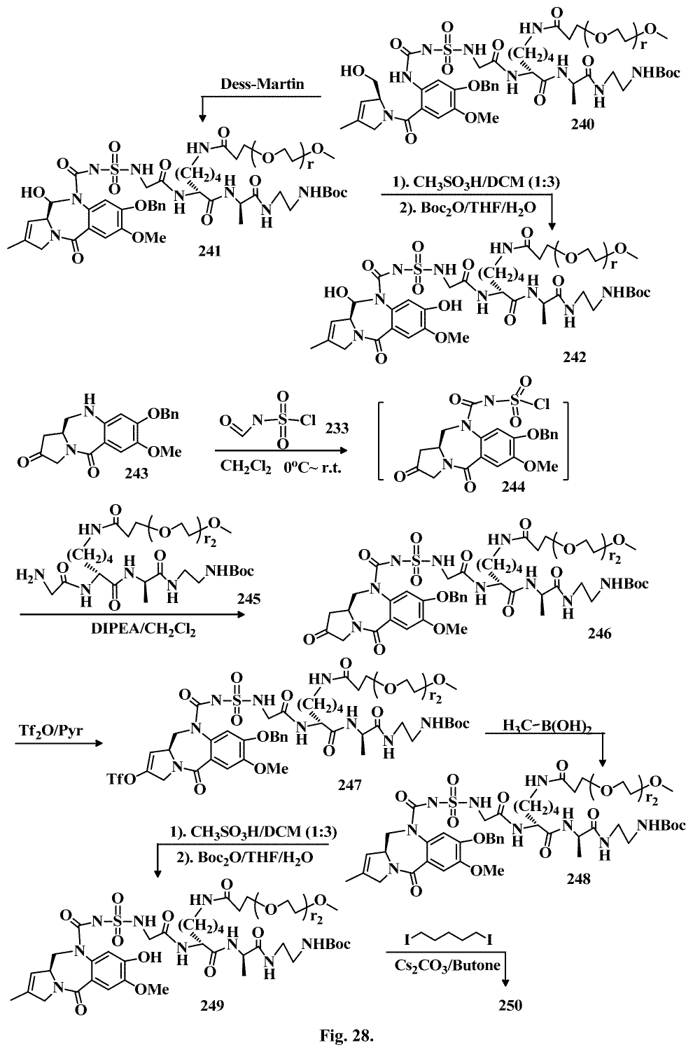

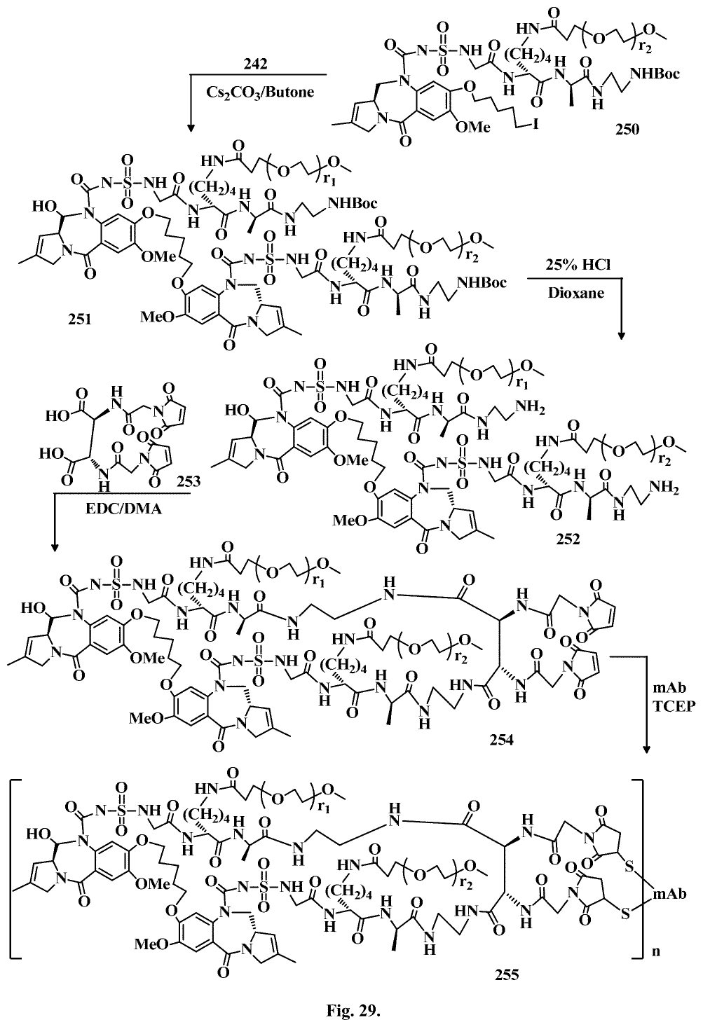

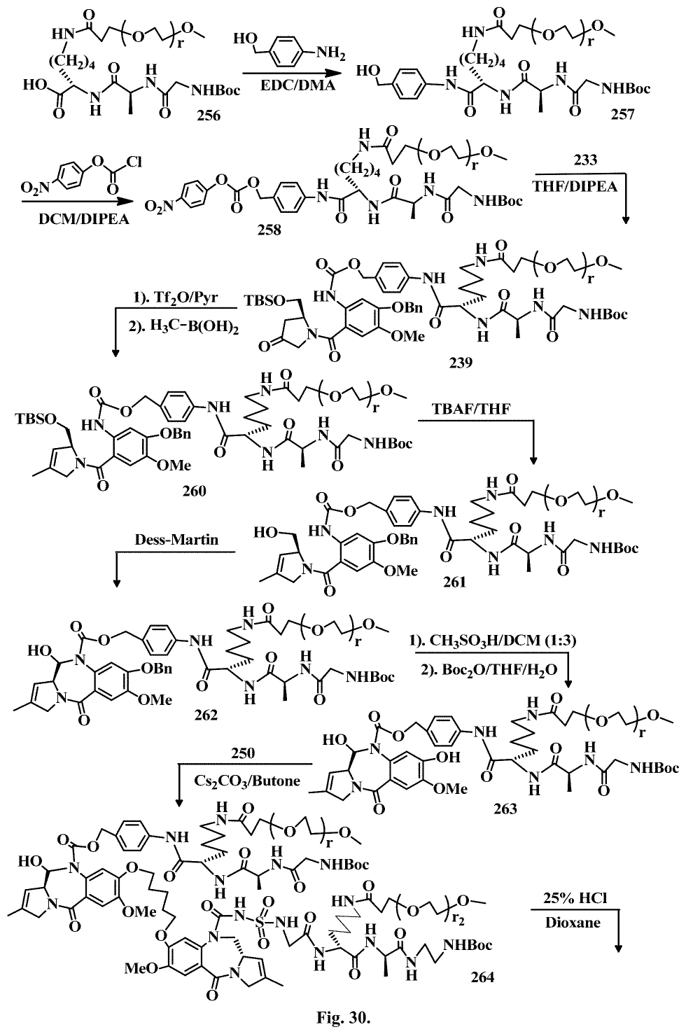

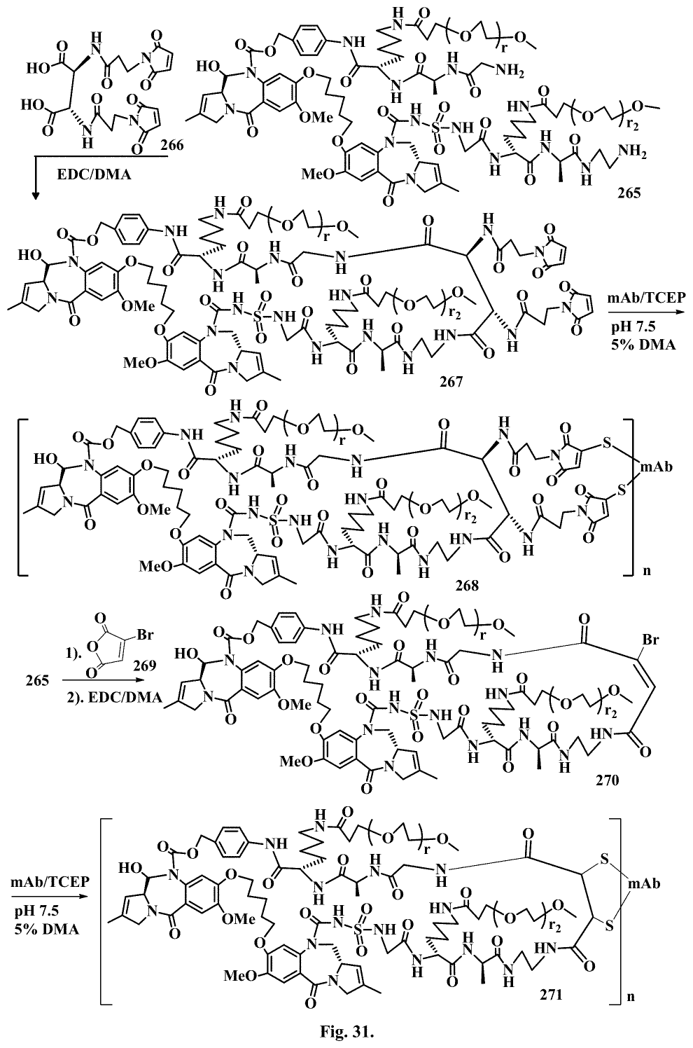

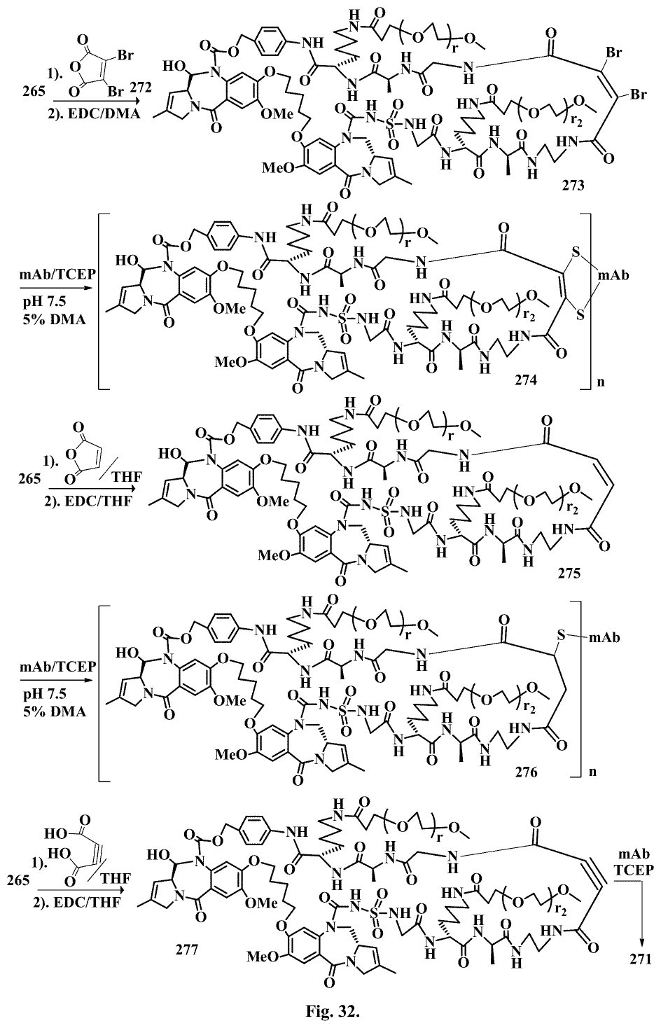

Other further exemplary methods for preparing ADC are described in FIGS. 1-32 and examples in the description of the patent.

Application of Conjugates of Cell-Binding Agent with Cross-Linked PBD Dimers.

The cell-binding agent-cross-linked PBD dimer conjugate, preferably antibody-cross-linked PBD dimer conjugates (PBD dimer ADC) of the present invention may be used to treat various diseases or disorders, e.g. characterized by the overexpression of a tumor antigen. Exemplary conditions or hyperproliferative disorders include benign or malignant tumors; leukemia and lymphoid malignancies. Others include neuronal, glial, astrocytal, hypothalamic, glandular, macrophagal, epithelial, stromal, blastocoelic, inflammatory, angiogenic and immunologic, including autoimmune, disorders.

In specific embodiment, the conjugates of the invention are used in accordance with the compositions and methods of the invention for the treatment of cancers. The cancers include, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproliferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor

In another specific embodiment, the compounds and the conjugates of the invention are used in accordance with the compositions and methods of the invention for the treatment or prevention of an autoimmune disease. The autoimmune diseases include, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaff s encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barré syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogammaglobulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Meniere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjögren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome

In another specific embodiment, a binding molecule used for the conjugate for the treatment or prevention of an autoimmune disease includes, but are not limited to, anti-elastin antibody; Abys against epithelial cells antibody; Anti-Basement Membrane Collagen Type IV Protein antibody; Anti-Nuclear Antibody; Anti ds DNA; Anti ss DNA, Anti Cardiolipin Antibody IgM, IgG; anti-celiac antibody; Anti Phospholipid Antibody IgK, IgG; Anti SM Antibody; Anti Mitochondrial Antibody; Thyroid Antibody; Microsomal Antibody, T-cells antibody; Thyroglobulin Antibody, Anti SCL-70; Anti-Jo; Anti-U.sub.1RNP; Anti-La/SSB; Anti SSA; Anti SSB; Anti Perital Cells Antibody; Anti Histones; Anti RNP; C-ANCA; P-ANCA; Anti centromere; Anti-Fibrillarin, and Anti GBM Antibody, Anti-ganglioside antibody; Anti-Desmogein 3 antibody; Anti-p62 antibody; Anti-sp100 antibody; Anti-Mitochondrial(M2) antibody; Rheumatoid factor antibody; Anti-MCV antibody; Anti-topoisomerase antibody; Anti-neutrophil cytoplasmic (cANCA) antibody.

In certain preferred embodiments, the binding molecule for the conjugate in the present invention, can bind to either a receptor or a receptor complex expressed on an activated lymphocyte which is associated with an autoimmune disease. The receptor or receptor complex can comprise an immunoglobulin gene superfamily member (e.g. CD2, CD3, CD4, CD8, CD19, CD20, CD22, CD28, CD30, CD33, CD37, CD38, CD56, CD79, CD79b, CD90, CD152/CTLA-4, PD-1, or ICOS), a TNF receptor superfamily member (e.g. CD27, CD40, CD95/Fas, CD134/OX40, CD137/4-1BB, INF-R1, TNFR-2, RANK, TACI, BCMA, osteoprotegerin, Apo2/TRAIL-R1, TRAIL-R2, TRAIL-R3, TRAIL-R4, and APO-3), an integrin, a cytokine receptor, a chemokine receptor, a major histocompatibility protein, a lectin (C-type, S-type, or I-type), or a complement control protein.

In another specific embodiment, useful binding ligands that are immunospecific for a viral or a microbial antigen are humanized or human monoclonal antibodies. As used herein, the term “viral antigen” includes, but is not limited to, any viral peptide, polypeptide protein (e.g. HIV gp120, HIV nef, RSV F glycoprotein, influenza virus neuramimidase, influenza virus hemagglutinin, HTLV tax, herpes simplex virus glycoprotein (e.g. gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., a bacterial, fungi, pathogenic protozoa, or yeast polypeptide including, e.g., LPS and capsular polysaccharide 5/8) that is capable of eliciting an immune response. Examples of antibodies available 1 for the viral or microbial infection include, but are not limited to, Palivizumab which is a humanized anti-respiratory syncytial virus monoclonal antibody for the treatment of RSV infection; PRO542 which is a CD4 fusion antibody for the treatment of HIV infection; Ostavir which is a human antibody for the treatment of hepatitis B virus; PROTVIR which is a humanized IgG.sub.1 antibody for the treatment of cytomegalovirus; and anti-LPS antibodies.

The binding molecules-cytotoxic agent conjugates of this invention can be used in the treatment of infectious diseases. These infectious diseases include, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcanobacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioidomycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome, Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsialpox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuberculosis infection, Yersiniosis, Yellow fever, Zygomycosis.

The binding molecules, more preferred antibodies described in this patent that are against pathogenic strains include, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4)—Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157.H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomyxoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthoramycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

Other antibodies as a binding ligand in this invention for treatment of viral disease include, but are not limited to, antibodies against antigens of pathogenic viruses, including as examples and not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytial virus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

Formulation and Application of the Conjugates.

The conjugates of the patent application are formulated to liquid, or suitable to be lyophilized and subsequently be reconstituted to a liquid formulation. A liquid formulation comprising 0.1 g/L˜300 g/L of concentration of a conjugate of the patent application as an active ingredient for delivery to a patient without high levels of antibody aggregation may include one or more polyols (e.g. sugars), a buffering agent with pH 4.5 to 7.5, a surfactant (e.g. polysorbate 20 or 80), an antioxidant (e.g. ascorbic acid and/or methionine), a tonicity agent (e.g. mannitol, sorbitol or NaCl), chelating agents such as EDTA; metal complexes (e.g. Zn-protein complexes); biodegradable polymers such as polyesters; a preservative (e.g. benzyl alcohol) and/or a free amino acid.

In a preferred embodiment, the conjugate of the invention in vivo clinical use will be supplied as solutions or as a lyophilized solid (such as powder) that can be redissolved in sterile water for injection. The conjugate in a liquid formula or in the formulated lyophilized powder may take up 0.01%-99% by weight as major gradient in the formulation. The rest of the formulation is excipients which are comprised of one or more of the following compounds: 0.5%˜25% of buffering reagents, 0%20% of polyols, 0%˜2.0% of surfactants, 0%˜5% of preservatives, 0%˜30% of Amino acids or bulky compounds, 0%˜5% of Antioxidants, 0%˜0.3% chelating agents.

Suitable buffering agents for use in the formulations include, but are not limited to, organic acid salts such as salts of citric acid, ascorbic acid, gluconic acid, carbonic acid, tartaric acid, succinic acid, acetic acid or phthalic acid; Tris, tromethamine (tris(hydroxymethyl)-aminomethane) hydrochloride, or phosphate buffer. In addition, amino acid components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-argine succinate, etc. The formulations of the buffers have a pH of 4.5 to pH 7.5, preferably from about 4.5 to about 6.5, more preferably from about 5.0 to about 6.2. In some embodiments, the concentration of the organic acid salts in the buffer is from about 10 mM to about 500 mM.

A “polyol” that may optionally be included in the formulation is a substance with multiple hydroxyl groups. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Polyols include sugars (reducing and nonreducing sugars), sugar alcohols and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and its metallic salts thereof. Preferably, a nonreducing sugar: sucrose or trehalose at a concentration of about from 0.01% to 15% is chosen in the formulation, wherein trehalose being preferred over sucrose, because of the solution stability of trehalose.

A surfactant optionally in the formulations is selected from polysorbate (polysorbate 20, polysorbate 40, polysorbate 65, polysorbate 80, polysorbate 81, polysorbate 85 and the like); poloxamer (e.g. poloxamer 188, poly(ethylene oxide)-poly(propylene oxide), poloxamer 407 or polyethylene-polypropylene glycol and the like); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-cocamidopropyl-, linoleamidopropyl-, myristamidopropyl-, palmidopropyl-, or isostearamido-propyl-betaine (e.g. lauroamidopropyl); myristamidopropyl-, palmidopropyl-, or isostearamido-propyl-dimethylamine; sodium methyl cocoyl-, or disodium methyl oleyl-taurate; dodecyl betaine, dodecyl dimethylamine oxide, cocamidopropyl betaine and coco ampho glycinate; and the MONAQUAT™ series (e.g. isostearyl ethylimidonium ethosulfate); polyethyl glycol, polypropyl glycol, and copolymers of ethylene and propylene glycol (e.g. Pluronics, PF68 etc.); etc. Preferred surfactants are polyoxyethylene sorbitan fatty acid esters e.g. polysorbate 20, 40, 60 or 80 (Tween 20, 40, 60 or 80). The concentration of a surfactant is range from 0.0001% to about 1.0%. In certain embodiments, the surfactant concentration is from about 0.01% to about 0.1%. In one embodiment, the surfactant concentration is about 0.02%.

A “preservative” optionally in the formulations is a compound that essentially reduces bacterial action therein. Examples of potential preservatives include octadecyldimethylbenzyl ammonium chloride, hexamethonium chloride, benzalkonium chloride (a mixture of alkylbenzyldimethyl-ammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The preservative is less than 5% in the formulation. Preferably 0.01% to 1%. In one embodiment, the preservative herein is benzyl alcohol.

Suitable free amino acids optionally for use in the formulation, but are not limited to, are arginine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as arginine-HCl. The concentration of an amino acid is range from 0.0001% to about 15.0%. Preferably 0.01% to 5%.

The formulations can optionally comprise methionine or ascorbic acid as an antioxidant at a concentration of about from 0.01 mg/ml to 5 mg/ml; The formulations can optionally comprise chelating agent, e.g., EDTA, EGTA, etc., at a concentration of about from 0.01 mM to 2 mM.

The final formulation may be adjusted to the preferred pH with an adjust agent (e.g. an acid, such as HCl, H₂SO₄, acetic acid, H₃PO₄, citric acid, etc., or a base, such as NaOH, KOH, NH₄OH, ethanolamine, diethanolamine or triethanol amine, sodium phosphate, potassium phosphate, trisodium citrate, tromethamine, etc.) and the formulation should be controlled “isotonic” which is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

Other excipients which may be useful in either a liquid or lyophilized formulation of the patent application include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol, phenol, polyhydric alcohols, or polyalcohols, hydrogenated forms of carbohydrate having a carbonyl group reduced to a primary or secondary hydroxyl group.

Other contemplated excipients, which may be utilized in the aqueous pharmaceutical compositions of the patent application include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4^{th} edition, Rowe et al., Eds., American Pharmaceuticals Association (2003); and Remington: the Science and Practice of Pharmacy, 21^{st} edition, Gennaro, Ed., Lippincott Williams & Wilkins (2005).

In order to reduce patient pain during the injection of the formulation, a local analgesic agent can be used along with or prior to the injection of the formulation. Commonly used analgesics are: benzyl alcohol (0.01%˜1%), Procaine hydrochloride (0.2%˜2.0%), lidocaine hydrochloride (0.2%˜2.0%), 2-tri-chloromethyl-2-propanol (0.3%˜0.5%), tramadol, morphine, morphine sulfate, hydromorphone, oxycodone hydrochloride, dobutamine, gabapentin, cyclobenzaprine, trazodone, clonidine, codeine.

The conjugates formulation of the patent application can be prepared as a pre-filled syringe solution, or a freezing-dry powder, or through high-efficient spray drying powder. A pharmaceutical container or a vessel is used to hold the pharmaceutical formulation of the conjugates. The vessel is a vial, bottle, pre-filled syringe, or pre-filled auto-injector syringe.

In a further embodiment, the invention provides a method for preparing a formulation comprising the steps of: (a) lyophilizing the formulation comprising the conjugates, excipients, and a buffer system to a powder; and (b) reconstituting the lyophilized mixture of step (a) in a reconstitution medium such that the reconstituted formulation is stable. The formulation of step (a) may further comprise a stabilizer and one or more excipients selected from a group comprising bulking agent, salt, surfactant and preservative as hereinabove described. As reconstitution media several diluted organic acids or water, i.e. sterile water, bacteriostatic water for injection (BWFI) or may be used. The reconstitution medium may be selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 10 to about 250 mM.

A liquid pharmaceutical formulation of the conjugates of the patent application should exhibit a variety of pre-defined characteristics. One of the major concerns in liquid drug products is stability, as proteins/antibodies tend to form soluble and insoluble aggregates during manufacturing and storage. In addition, various chemical reactions can occur in solution (deamidation, oxidation, clipping, isomerization etc.) leading to an increase in degradation product levels and/or loss of bioactivity. Preferably, a conjugate in either liquid or loyphilizate formulation should exhibit a shelf life of more than 18 months at 25° C. More preferred a conjugate in either liquid or loyphilizate formulation should exhibit a shelf life of more than 24 months at 25° C. Most preferred liquid formulation should exhibit a shelf life of about 24 to 36 months at 2-8° C. and the loyphilizate formulation should exhibit a shelf life of about preferably up to 60 months at 2-8° C. Both liquid and loyphilizate formulations should exhibit a shelf life for at least two years at −20° C., or −70° C.

In certain embodiments, the formulation is stable following freezing (e.g., −20° C., or −70° C.) and thawing of the formulation, for example following 1, 2 or 3 cycles of freezing and thawing. Stability can be evaluated qualitatively and/or quantitatively in a variety of different ways, including evaluation of drug/antibody(protein) ratio and aggregate formation (for example using UV, size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis, or matrix-assisted laser desorption ionization/time-of-flight mass spectrometry (MALDI/TOF MS), or HPLC-MS/MS; SDS-PAGE analysis to compare reduced and intact antibody; peptide map (for example tryptic or LYS—C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A stable conjugate should also “retains its biological activity” in a pharmaceutical formulation, if the biological activity of the conjugate at a given time, e.g. 12 month, within about 20%, preferably about 10% (within the errors of the assay) of the biological activity exhibited at the time the pharmaceutical formulation was prepared as determined in an antigen binding assay, and/or in vitro, cytotoxic assay, for example.

Examples of suitable protocols of conjugate administration are as follows. Conjugates are given daily, weekly, biweekly, triweekly, once every four weeks or monthly for 8˜54 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg to 20 mg/kg of body weight per week, i.v. (range of 10 μg to 200 mg/kg per injection). 4˜54 weeks after treatment, the patient may receive a second course of treatment. Specific clinical protocols with regard to route of administration, excipients, diluents, dosages, times, etc., can be determined by the skilled clinicians.

Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumor cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 30 minutes to about 48 hours at about 37° C. The exact conditions of concentration and time of incubation (=dose) are readily determined by the skilled clinicians. After incubation the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

Examples of medical conditions that can be treated according to the in vivo or ex vivo methods of killing selected cell populations include malignancy of any types of cancer, autoimmune diseases, graft rejections, and infections (viral, bacterial or parasite).

Examples of medical conditions that can be treated according to the in vivo or ex vivo methods of killing selected cell populations include malignancy of any type including, for example, cancer of the lung, breast, colon, prostate, kidney, pancreas, ovary, and lymphatic organs; melanomas; autoimmune diseases, such as systemic lupus, rheumatoid arthritis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, AIDS, etc.; bacterial infection; and parasite infections, such as giardiasis, amoebiasis, schistosomiasis, and others as determined by one skilled in the art.

The identification of those subjects who are in need of treatment of herein-described diseases and conditions is well within the ability and knowledge of one skilled in the art. A veterinarian or a physician skilled in the art can readily identify, by the use of clinical tests, physical examination, medical/family history or biological and diagnostic tests, those subjects who are in need of such treatment.

A therapeutically effective amount can be readily determined by the attending diagnostician, as one skilled in the art, by the use of conventional techniques and by observing results obtained under analogous circumstances. In determining the therapeutically effective amount, a number of factors are considered by the attending diagnostician, including, but not limited to: the species of subject; its size, age, and general health; the specific disease involved; the degree of involvement or the severity of the disease; the response of the individual subject; the particular compound administered; the mode of administration; the bioavailability characteristic of the preparation administered; the dose regimen selected; the use of concomitant medication; and other relevant circumstances.

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates of this invention may be provided in an aqueous physiological buffer solution containing 0.1 to 30% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 20 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

The cell binding agent-cytotoxic agent conjugates of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily dose ranges are from 0.01 to 100 mg/kg of body weight. By way of general guidance, unit doses for humans range from 1 mg to 3000 mg per day, weekly, biweekly, or triweekly. Preferably the unit dose range is from 1 to 500 mg administered one to two times weekly, biweekly, or triweekly. and even more preferably from 10 mg to 500 mg, biweekly, or triweekly. Conjugates provided herein can be formulated into pharmaceutical compositions by admixture with one or more pharmaceutically acceptable excipients. Such unit dose compositions may be prepared for use by I.V. or oral administration, particularly in the form of powders, tablets, simple capsules or soft gel capsules; or intranasally, particularly in the form of powders, nasal drops, or aerosols; or dermally, for example, topically in ointments, creams, lotions, gels or sprays, or via trans-dermal patches. The compositions may conveniently be administered in unit dosage form and may be prepared by any of the methods well known in the pharmaceutical art, for example, as described in Remington: The Science and Practice of Pharmacy, 21^{th} ed.; Lippincott Williams & Wilkins: Philadelphia, Pa., 2005. Preferred formulations include pharmaceutical compositions in which a compound of the present invention is formulated for oral or parenteral administration. For oral administration, tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal. Preferred tablets contain lactose, cornstarch, magnesium silicate, croscarmellose sodium, povidone, magnesium stearate, or talc in any combination. Liquid preparations for parenteral administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, vegetable oils such as olive oil, and organic esters such as ethyl oleate. Aqueous carriers include mixtures of alcohols and water, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like. Other potentially useful parenteral delivery systems for these active compounds include ethylene-vinyl acetate copolymer particles, osmotic pumps, implantable infusion systems, and liposomes.

Alternative modes of administration include formulations for inhalation, which include such means as dry powder, aerosol, or drops. They may be aqueous solutions containing, for example, polyoxyethylene-9-lauryl ether, glycocholate and deoxycholate, or oily solutions for administration in the form of nasal drops, or as a gel to be applied intranasally. Formulations for buccal administration include, for example, lozenges or pastilles and may also include a flavored base, such as sucrose or acacia, and other excipients such as glycocholate. Formulations suitable for rectal administration are preferably presented as unit-dose suppositories, with a solid based carrier, such as cocoa butter, and may include a salicylate. Formulations for topical application to the skin preferably take the form of an ointment, cream, lotion, paste, gel, spray, aerosol, or oil. Carriers which can be used include petroleum jelly, lanolin, polyethylene glycols, alcohols, or their combinations. Formulations suitable for transdermal administration can be presented as discrete patches and can be lipophilic emulsions or buffered, aqueous solutions, dissolved and/or dispersed in a polymer or an adhesive.

In a specific embodiment, the cell binding agent-cytotoxic agent conjugates of this invention are administered concurrently with the other known or will be known therapeutic agents such as the chemotherapeutic agent, the radiation therapy, immunotherapy agents, autoimmune disorder agents, anti-infectious agents or the other antibody-drug conjugates, resulting in a synergistic effect for effective treatment or prevention of a cancer, or an autoimmune disease, or an infectious disease. In another specific embodiment, the synergistic drugs or radiation therapy are administered prior or subsequent to administration of a conjugate, in one aspect at least an hour, 12 hours, a day, a week, two weeks, three weeks, a month, in further aspects several months, prior or subsequent to administration of a conjugate of the invention.

The synergistic agents are preferably selected from one or several of the following drugs:

1). Chemotherapeutic agents: a). Alkylating agents: such as [Nitrogen mustards: (chlorambucil, cyclophosphamide, ifosfamide, mechlorethamine, melphalan, trofosfamide); Nitrosoureas: (carmustine, lomustine); Alkylsulphonates: (busulfan, treosulfan); Triazenes: (dacarbazine); Platinum containing compounds: (carboplatin, cisplatin, oxaliplatin)]; b). Plant Alkaloids: such as [Vinca alkaloids: (vincristine, vinblastine, vindesine, vinorelbine); Taxoids: (paclitaxel, docetaxol)]; c). DNA Topoisomerase Inhibitors: such as [Epipodophyllins: (9-aminocamptothecin, camptothecin, crisnatol, etoposide, etoposide phosphate, irinotecan, teniposide, topotecan,); Mitomycins: (mitomycin C)]; d). Anti-metabolites: such as {[Anti-folate: DHFR inhibitors: (methotrexate, trimetrexate); IMP dehydrogenase Inhibitors: (mycophenolic acid, tiazofurin, ribavirin, EICAR); Ribonucleotide reductase Inhibitors: (hydroxyurea, deferoxamine)]; [Pyrimidine analogs: Uracil analogs: (5-Fluorouracil, doxifluridine, floxuridine, ratitrexed(Tomudex)); Cytosine analogs: (cytarabine, cytosine arabinoside, fludarabine); Purine analogs: (azathioprine, mercaptopurine, thioguanine)]}; e). Hormonal therapies: such as {Receptor antagonists: [Anti-estrogen: (megestrol, raloxifene, tamoxifen); LHRH agonists: (goscrclin, leuprolide acetate); Anti-androgens: (bicalutamide, flutamide)]; Retinoids/Deltoids: [Vitamin D3 analogs: (CB 1093, EB 1089 KH 1060, cholecalciferol, ergocalciferol); Photodynamic therapies: (verteporfin, phthalocyanine, photosensitizer Pc4, demethoxyhypocrellin A); Cytokines: (Interferon-alpha, Interferon-gamma, tumor necrosis factor (TNFs), human proteins containing a TNF domain)]}; f). Kinase inhibitors, such as BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR₂), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib; g). Others: such as gemcitabine, epoxomicins (e.g. carfilzomib), bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors (such as Lovastatin), Dopaminergic neurotoxins (such as 1-methyl-4-phenylpyridinium ion), Cell cycle inhibitors (such as staurosporine), Actinomycins (such as Actinomycin D, dactinomycin), Bleomycins (such as bleomycin A2, bleomycin B2, peplomycin), Anthracyclines (such as daunorubicin, doxorubicin (adriamycin), idarubicin, epirubicin, pirarubicin, zorubicin, mtoxantrone, MDR inhibitors (such as verapamil), Ca²⁺ ATPase inhibitors (such as thapsigargin), vismodegib, Histone deacetylase inhibitors (Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A. More detail lists of known and will be known anti-cancer drugs that can be used as a combination therapy (a synergistic effect) with the compounds and conjugates of the invention can be seen in National Cancer Institute (US) website (www.cancer.gov; www.cancer.gov/cancertopics/druginfo/alphalist), American Cancer Society (www.cancer.org/treatment/index) and Cancer Research UK (www.cancerrearchuk.org; (www.cancerresearchuk.org/cancer-help/about-cancer/treatment/cancer-drugs/)

2). An anti-autoimmune disease agent includes, but is not limited to, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.

3). An anti-infectious disease agent includes, but is not limited to, a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin; b). Amphenicols: azidamfenicol, chloramphenicol, florfenicol, thiamphenicol; c). Ansamycins: geldanamycin, herbimycin; d). Carbapenems: biapenem, doripenem, ertapenem, imipenem/cilastatin, meropenem, panipenem; e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef); f). Glycopeptides: bleomycin, vancomycin (oritavancin, telavancin), teicoplanin (dalbavancin), ramoplanin; g). Glycylcyclines: e.g. tigecycline; g). β-Lactamase inhibitors: penam (sulbactam, tazobactam), clavam (clavulanic acid); i). Lincosamides: clindamycin, lincomycin; j). Lipopeptides: daptomycin, A54145, calcium-dependent antibiotics (CDA); k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin; l). Monobactams: aztreonam, tigemonam; m). Oxazolidinones: linezolid; n). Penicillins: amoxicillin, ampicillin (pivampicillin, hetacillin, bacampicillin, metampicillin, talampicillin), azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin; o). Polypeptides: bacitracin, colistin, polymyxin B; p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin; q). Streptogramins: pristinamycin, quinupristin/dalfopristin); r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole); s). Steroid antibacterials: e.g. fusidic acid; t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (e.g. tigecycline); u). Other types of antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (e.g. fosfomycin), nitrofurantoin, paclitaxel, platensimycin, pyrazinamide, quinupristin/dalfopristin, rifampicin (rifampin), tazobactam tinidazole, uvaricin;

4). Anti-viral drugs: a). Entry/fusion inhibitors: aplaviroc, maraviroc, vicriviroc, gp41 (enfuvirtide), PRO 140, CD4 (ibalizumab); b). Integrase inhibitors: raltegravir, elvitegravir, globoidnan A; c). Maturation inhibitors: bevirimat, vivecon; d). Neuraminidase inhibitors: oseltamivir, zanamivir, peramivir; e). Nucleosides &_nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′, 3′-dideoxynucleoside analogues (e.g. 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (e.g. β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT); f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine; g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir; h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.

5). Other immunotherapy drugs: e.g. imiquimod, interferons (e.g. a, p), granulocyte colony-stimulating factors, cytokines, Interleukins (IL-1 IL-35), antibodies (e.g. trastuzumab, pertuzumab, bevacizumab, cetuximab, panitumumab, infliximab, adalimumab, basiliximab, daclizumab, omalizumab), Protein-bound drugs (e.g., Abraxane), an antibody conjugated with drugs selected from calicheamicin derivative, of maytansine derivatives (DM1 and DM4), CC-1065 and duocarmycin minor groove binders, potent taxol derivatives, doxorubicin, auristatin antimitotic drugs (e.g. Trastuzumab-DM1, Inotuzumab ozogamicin, Brentuximab vedotin, Glembatumumab vedotin, lorvotuzumab mertansine, AN-152 LMB2, TP-38, VB4-845, Cantuzumab mertansine, AVE9633, SAR3419, CAT-8015 (anti-CD22), IMGN388, milatuzumab-doxorubicin, SGN-75 (anti-CD70), Anti-CD22-MCC-DM1, IMGN853, Anti-CD22-MMAE, Anti-CD22-MMAF, Anti-CD22-calicheamicin.

In a further embodiment, the synergistic agents are selected from one or several of the following drugs: Abatacept, Abiraterone acetate, Abraxane, Acetaminophen/hydrocodone, Acalabrutinib, aducanumab, Adalimumab, ADXS31-142, ADXS-HER2, afatinib dimaleate, aldesleukin, alectinib, alemtuzumab, Alitretinoin, ado-trastuzumab emtansine, Amphetamine/dextroamphetamine, anastrozole, Aripiprazole, anthracyclines, Aripiprazole, Atazanavir, Atezolizumab, Atorvastatin, Avelumab, Axicabtagene ciloleucel, axitinib, belinostat, BCG Live, Bevacizumab, bexarotene, blinatumomab, Bortezomib, bosutinib, brentuximab vedotin, brigatinib, Budesonide, Budesonide/formoterol, Buprenorphine, Cabazitaxel, Cabozantinib, capmatinib, Capecitabine, carfilzomib, chimeric antigen receptor-engineered T (CAR-T) cells, Celecoxib, ceritinib, Cetuximab, Chidamide, Ciclosporin, Cinacalcet, crizotinib, Cobimetinib, Cosentyx, crizotinib, CTL019, Dabigatran, dabrafenib, dacarbazine, daclizumab, dacomotinib, daptomycin, Daratumumab, Darbepoetin alfa, Darunavir, dasatinib, denileukin diftitox, Denosumab, Depakote, Dexlansoprazole, Dexmethylphenidate, Dexamethasone, DigniCap Cooling System, Dinutuximab, Doxycycline, Duloxetine, Duvelisib, durvalumab, elotuzumab, Emtricitabine/Rilpivirine/Tenofovir, disoproxil fumarate, Emtricitbine/tenofovir/efavirenz, Enoxaparin, ensartinib, Enzalutamide, Epoetin alfa, erlotinib, Esomeprazole, Eszopiclone, Etanercept, Everolimus, exemestane, everolimus, exenatide ER, Ezetimibe, Ezetimibe/simvastatin, Fenofibrate, Filgrastim, fingolimod, Fluticasone propionate, Fluticasone/salmeterol, fulvestrant, gazyva, gefitinib, Glatiramer, Goserelin acetate, Icotinib, Imatinib, Ibritumomab tiuxetan, ibrutinib, idelalisib, ifosfamide, Infliximab, imiquimod, ImmuCyst, Immuno BCG, iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144, lorlatinib, Memantine, Methylphenidate, Metoprolol, Mekinist, mericitabine/Rilpivirine/Tenofovir, Modafinil, Mometasone, Mycidac-C, Necitumumab, neratinib, Nilotinib, niraparib, Nivolumab, ofatumumab, obinutuzumab, olaparib, Olmesartan, Olmesartan/hydrochlorothiazide, Omalizumab, Omega-3 fatty acid ethyl esters, Oncorine, Oseltamivir, Osimertinib, Oxycodone, palbociclib, Palivizumab, panitumumab, panobinostat, pazopanib, pembrolizumab, PD-1 antibody, PD-L1 antibody, Pemetrexed, pertuzumab, Pneumococcal conjugate vaccine, pomalidomide, Pregabalin, ProscaVax, Propranolol, Quetiapine, Rabeprazole, radium 223 chloride, Raloxifene, Raltegravir, ramucirumab, Ranibizumab, regorafenib, Rituximab, Rivaroxaban, romidepsin, Rosuvastatin, ruxolitinib phosphate, Salbutamol, savolitinib, semaglutide, Sevelamer, Sildenafil, siltuximab, Sipuleucel-T, Sitagliptin, Sitagliptin/metformin, Solifenacin, solanezumab, Sonidegib, Sorafenib, Sunitinib, tacrolimus, tacrimus, Tadalafil, tamoxifen, Tafinlar, Talimogene laherparepvec, talazoparib, Telaprevir, talazoparib, Temozolomide, temsirolimus, Tenofovir/emtricitabine, tenofovir disoproxil fumarate, Testosterone gel, Thalidomide, TICE BCG, Tiotropium bromide, Tisagenlecleucel, toremifene, trametinib, Trastuzumab, Trabectedin (ecteinascidin 743), trametinib, tremelimumab, Trifluridine/tipiracil, Tretinoin, Uro-BCG, Ustekinumab, Valsartan, veliparib, vandetanib, vemurafenib, venetoclax, vorinostat, ziv-aflibercept, Zostavax, and their analogs, derivatives, pharmaceutically acceptable salts, carriers, diluents, or excipients thereof, or a combination above thereof.

The invention is further illustrated but not restricted by the description in the following examples.

### EXAMPLES

The invention is further described in the following examples, which are not intended to limit the scope of the invention. Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ), or The Shanghai Cell Culture Institute of Chinese Acadmy of Science, unless otherwise specified. Cell culture reagents were obtained from Invitrogen Corp., unless otherwise specified. All anhydrous solvents were commercially obtained and stored in Sure-seal bottles under nitrogen. All other reagents and solvents were purchased as the highest grade available and used without further purification. The preparative HPLC separations were performed with Varain PreStar HPLC. NMR spectra were recorded on Bruker 500 MHz Instrument. Chemical shifts (delta) are reported in parts per million (ppm) referenced to tetramethylsilane at 0.00 and coupling constants (J) are reported in Hz. The mass spectral data were acquired on a Waters Xevo QTOF mass spectrum equipped with Waters Acquity UPLC separations module and Acquity TUV detector.

### Example 1. Synthesis of Di-tert-butyl 1,2-bis(2-(tert-butoxy)-2-oxoethyl)hydrazine-1,2-dicarboxylate

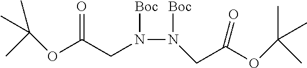 [SMILES: CC(C)(C)OC(=O)CN(C(=O)OC(C)(C)C)N(CC(=O)OC(C)(C)C)C(=O)OC(C)(C)C]

To di-tert-butyl hydrazine-1,2-dicarboxylate (8.01 g, 34.4 mmol) in DMF (150 ml) was added NaH (60% in oil, 2.76 g, 68.8 mmol). After stirred at RT for 30 min, tert-butyl 2-bromoacetate (14.01 g, 72.1 mmol) was added. The mixture was stirred overnight, quenched with addition of methanol (3 ml), concentrated, diluted with EtOAc (100 ml) and water (100 ml), separated, and the aqueous layer was extracted with EtOAc (2×50 ml). The organic layers were combined, dried over MgSO₄, filtered, evaporated, and purified by SiO₂ column chromatography (EtOAc/Hexane 1:5 to 1:3) to afforded the title compound (12.98 g, 82% yield) as a colorless oil. MS ESI m/z calcd for C₂₂H₄₁N₂O₈ [M+H]⁺ 461.28, found 461.40.

### Example 2. Synthesis of 2,2′-(hydrazine-1,2-diyl)diacetic Acid

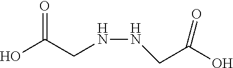

Di-tert-butyl 1,2-bis(2-(tert-butoxy)-2-oxoethyl)hydrazine-1,2-dicarboxylate (6.51 g, 14.14 mmol) in 1,4-dioxane (40 ml) was added HCl (12 M, 10 ml). The mixture was stirred for 30 min, diluted with dioxane (20 ml) and toluene (40 ml), evaporated and co-evaporated with dioxane (20 ml) and toluene (40 ml) to dryness to afford the crude title product for the next step without further production (2.15 g, 103% yield, ˜93% pure). MS ESI m/z calcd for C₄H₉N₂O₄ [M+H]⁺ 149.05, found 149.40.

### Example 3. Synthesis of 2,2′-(1,2-bis((benzyloxy)carbonyl)hydrazine-1,2-diyl)diacetic Acid

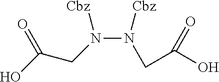 [SMILES: O=C(O)CN(C(=O)OCc1ccccc1)N(CC(=O)O)C(=O)OCc1ccccc1]

To a solution of 2,2′-(hydrazine-1,2-diyl)diacetic acid (1.10 g, 7.43 mmol) in the mixture of THE (200 ml) and NaH₂PO₄ (0.1 M, 250 ml, pH 8.0) was added benzyl carbonochloridate (5.01 g, 29.47 mmol) in 4 portions in 2 h. The mixture was stirred for another 6 h, concentrated and purified on SiO₂ column eluted with H₂O/CH₃CN (1:9) containing 1% formic acid to afford the title compound (2.26 g, 73% yield, ˜95% pure). MS ESI m/z calcd for C₂₀H₂₁N₂O₈ [M+H]⁺ 417.12, found 417.40.

### Example 4. Synthesis of Dibenzyl 1,2-bis(2-chloro-2-oxoethyl)hydrazine-1,2-dicarboxylate

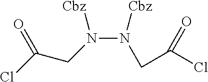 [SMILES: O=C(Cl)CN(C(=O)OCc1ccccc1)N(CC(=O)Cl)C(=O)OCc1ccccc1]

2,2′-(1,2-bis((benzyloxy)carbonyl)hydrazine-1,2-diyl)diacetic acid (350 mg, 0.841 mmol) in dichloroethane (30 ml) was added (COCl)₂ (905 mg, 7.13 mmol), followed by addition of 0.030 ml of DMF. After stirred at RT for 2 h, the mixture was diluted with toluene, concentrated and co-evaporated with dichloroethane (2×20 ml) and toluene (2×15 ml) to dryness to afford the title crude product (which is not stable) for the next step without further purification (365 mg, 96% yield). MS ESI m/z calcd for C₂₀H₁₉C₁₂N₂O₆ [M+H]⁺ 453.05, found 453.50.

### Example 5. Synthesis of Di-tert-butyl 1,2-bis(2-(tert-butoxy)-2-oxoethyl)hydrazine-1,2-dicarboxylate

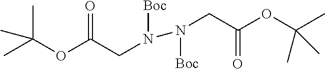 [SMILES: CC(C)(C)OC(=O)CN(C(=O)OC(C)(C)C)N(CC(=O)OC(C)(C)C)C(=O)OC(C)(C)C]

To a suspension of NaH (0.259 g, 6.48 mmol, 3.0 eq.) in anhydrous DMF (2 mL) at room temperature was added di-tert-butyl hydrazine-1,2-dicarboxylate (0.50 g, 2.16 mmol, 1.0 eq.) in anhydrous DMF (8 mL) in 10 minutes under nitrogen. The mixture was stirred at room temperature for 10 minutes and then cooled to 0° C. To which tert-butyl 2-bromoacetate (1.4 mL, 8.61 mmol, 4.0 eq.) was added dropwise. The resulting mixture was allowed to warm to room temperature and stirred overnight. Saturated ammonium chloride solution (100 mL) was added. The organic layer was separated and the aqueous layer was extracted with EtOAc (3×50 mL). The combined organic solution was washed with water and brine, dried over anhydrous Na₂SO₄, concentrated and purified by SiO₂ column chromatography (10:1 hexanes/EtOAc) to give the title compound as a colourless oil (0.94 g, 99.6% yield). ESI MS m/z [M+Na]⁺ 483.4.

### Example 6. Synthesis of Compound 2,2′-(hydrazine-1,2-diyl)diacetic Acid

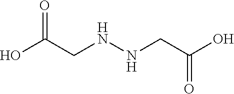

To a solution of di-tert-butyl 1,2-bis(2-(tert-butoxy)-2-oxoethyl)hydrazine-1,2-dicarboxylate (0.94 g, 2.04 mmol) in DCM (4 mL) at 0° C. was added TFA (4 mL). The reaction was stirred for 30 minutes and then warmed to room temperature and stirred overnight. The mixture was concentrated, diluted with DCM, and concentrated. This operation was repeated for three times to give a white solid. Trituration with DCM and a white solid was collected by filtration (0.232 g, 76.8% yield). ESI MS m/z [M+H]⁺ 149.2.

### Example 7. Synthesis of 2,2′-(1,2-bis(2-chloroacetyl)hydrazine-1,2-diyl)diacetic Acid

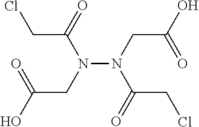 [SMILES: O=C(O)CN(C(=O)CCl)N(CC(=O)O)C(=O)CCl]

To a solution of 2,2′-(hydrazine-1,2-diyl)diacetic acid (0.232 g, 1.57 mmol, 1.0 eq.) in anhydrous THF (10 mL) at 0° C. was added 2-chloroacetyl chloride (0.38 mL, 4.70 mmol, 3.0 eq.) in 10 minutes. The reaction was warmed to room temperature and stirred overnight and concentrated. The residue was co-evaporated with THF for three times to give a white solid (0.472 g, theoretical yield). ESI MS m/z [M+H]⁺ 301.1.

### Example 8. Synthesis of Tert-butyl 2,8-dioxo-1,5-oxazocane-5-carboxylate

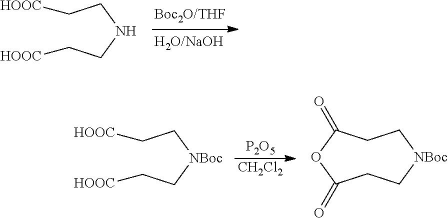

To a solution of 3,3′-azanediyldipropanoic acid (10.00 g, 62.08 mmol) in 1.0 M NaOH (300 ml) at 4° C. was added di-tert-butyl dicarbonate (22.10 g, 101.3 mmol) in 200 ml THF in 1 h. After addition, the mixture was kept to stirring for 2 h at 4° C. The mixture was carefully acidified to pH ˜4 with 0.2 M H₃PO₄, concentrated in vacuo, extracted with CH2Cl2, dried over Na2SO4, evaporated and purified with flash SiO2 chromatography eluted with AcOH/MeOH/CH₂Cl₂ (0.01:1:5) to afford 3,3′-((tert-butoxycarbonyl)azanediyl)dipropanoic acid (13.62 g, 84% yield). ESI MS m/z C₁₁H₁₉NO₆ [M+H]⁺, cacld. 262.27, found 262.40.

To a solution of 3,3′-((tert-butoxycarbonyl)azanediyl)dipropanoic acid (8.0 g, 30.6 mmol) in CH₂Cl₂ (500 ml) at 0° C. was added phosphorus pentoxide (8.70 g, 61.30 mmol). The mixture was stirred at 0° C. for 2 h and then r.t. for 1 h, filtered through short SiO₂ column, and rinsed the column with EtOAc/CH₂Cl₂ (1:6). The filtrate was concentrated and triturated with EtOAc/hexane to afford the title compound (5.64 g, 74% yield). ESI MS m/z C₁₁H₁₇NO₅ [M+H]⁺, cacld. 244.11, found 244.30.

### Example 9. Synthesis of Tert-Butyl 3-((benzyloxy)amino)propanoate

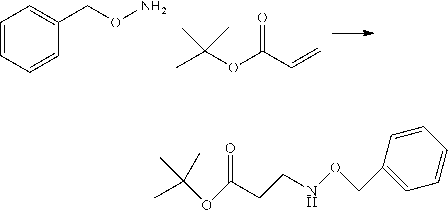

O-benzylhydroxylamine hydrochloride salt (10.0 g, 62.7 mmol) in THF (100 ml) was added Et₃N (15 ml) and tert-butyl acrylate (12.1 g, 94.5 mmol). The mixture was refluxed for overnight, concentrated and purified on SiO₂ column eluted with EtOAc/Hexane (1:4) to afford the title compound 3 (13.08 g, 83% yield). 1H NMR (CDCl₃) 7.49˜7.25 (m, 5H), 4.75 (s, 2H), 3.20 (t, J=6.4 Hz, 2H), 2.54 (t, J=6.4 Hz, 2H), 1.49 (s, 9H); ESI MS m/z+ C₁₄H₂₁NNaO₃ (M+Na), cacld. 274.15, found 274.20.

### Example 10. Synthesis of Tert-Butyl 3-(hydroxyamino)propanoate

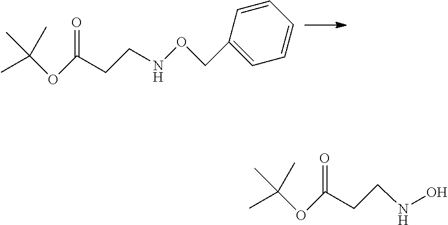 [SMILES: CC(C)(C)OC(=O)CCNOCc1ccccc1 ; CC(C)(C)OC(=O)CCNO]

tert-Butyl 3-((benzyloxy)amino)propanoate (13.0 g, 51.76 mmol) in methanol (100 ml) was added Pd/C (0.85 g, 10% Pd, 50% wet) in a hydrogenation vessel. After the system was evacuated under vacuum and placed under 2 atm of hydrogen gas, the reaction mixture was stirred overnight at room temperature. The crude reaction was passed through a short pad of Celite rinsing with ethanol, concentrated and purified on SiO₂ column eluted with MeOH/DCM (1:10˜1:5) to afford the title compound (7.25 g, 87% yield). 1H NMR (CDCl₃) 3.22 (t, J=6.4 Hz, 2H), 2.55 (t, J=6.4 Hz, 2H), 1.49 (s, 9H); ESI MS m/z+ C₇H₁₅NNaO₃ (M+Na), cacld. 184.10, found 184.30.

### Example 11. Synthesis of Tert-Butyl 3-((tosyloxy)amino)propanoate

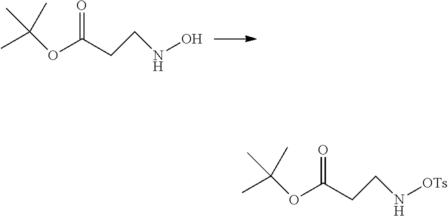 [SMILES: CC(C)(C)OC(=O)CCNO ; Cc1ccc(S(=O)(=O)ONCCC(=O)OC(C)(C)C)cc1]

Tert-butyl 3-(hydroxyamino)propanoate (5.10 g, 31.65 mmol) in the mixture of DCM (50 ml) and pyridine (20 ml) was added tosylate chloride (12.05 g, 63.42) at 4° C. After addition, the mixture was stirred at room temperature overnight, concentrated and purified on SiO₂ column eluted with EtOAc/DCM (1:10˜1:6) to afford the title compound (8.58 g, 86% yield). 1H NMR (CDCl₃) 7.81 (s, 2H), 7.46 (s, 2H), 3.22 (t, J=6.4 Hz, 2H), 2.55 (t, J=6.4 Hz, 2H), 2.41 (s, 3H), 1.49 (s, 9H); ESI MS m/z+ C₁₄H₂₁NNaO₅S (M+Na), cacld. 338.11, found 338.30.

### Example 12. Synthesis of Di-tert-Butyl 3,3′-(hydrazine-1,2-diyl)dipropanoate

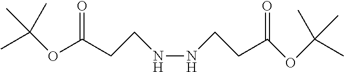 [SMILES: CC(C)(C)OC(=O)CCNNCCC(=O)OC(C)(C)C]

Tert-butyl 3-aminopropanoate (3.05 g, 21.01 mmol) in THE (80 ml) was added tert-Butyl 3-((tosyloxy)amino)propanoate (5.10 g, 16.18 mmol). The mixture was stirred at room temperature for 1 h and then 45° C. for 6 h. The mixture was concentrated and purified on SiO₂ column eluted with CH₃OH/DCM/Et₃N (1:12:0.01˜1:8:0.01) to afford the title compound (2.89 g, 62% yield). ESI MS m/z+ C₁₄H₂₈N₂NaO₄ (M+Na), cacld. 311.20, found 311.40.

### Example 13. Synthesis of Di-tert-Butyl 3,3′-(1,2-bis(3-(2,5-dioxo-2,5-dihydro-1H-pyrrol-1-yl)propanoyl)hydrazine-1,2-diyl)dipropanoate

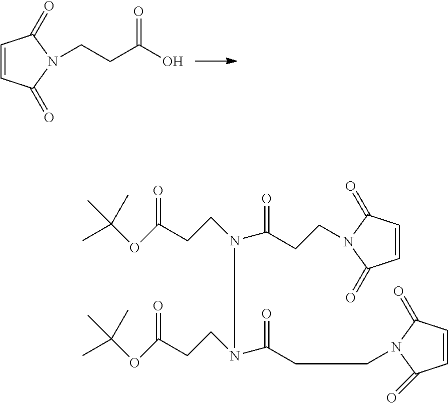 [SMILES: O=C(O)CCN1C(=O)C=CC1=O ; CC(C)(C)OC(=O)CCN(C(=O)CCN1C(=O)C=CC1=O)N(CCC(=O)OC(C)(C)C)C(=O)CCN1C(=O)C=CC1=O]

3-Maleido-propanoic acid (1.00 g, 5.91 mmol) in DCM (50 ml) was added oxalyl dichloride (2.70 g, 21.25 mmol) and DMF (50 μL). The mixture was stirred at room temperature for 2 h, evaporated, and co-evaporated with DCM/toluene to obtain crude 3-maleido-propanoic acid chloride. To the compound di-tert-Butyl 3,3′-(hydrazine-1,2-diyl)dipropanoate (0.51 g, 1.76 mmol) in the mixture of DCM (35 ml) was added the crude 3-maleido-propanoic acid chloride. The mixture was stirred for overnight, evaporated, concentrated and purified on SiO₂ column eluted with EtOAc/DCM (1:15˜1:8) to afford the title compound (738 mg, 71% yield). ESI MS m/z+ C₂₈H₃₈N₄NaO₁₀ (M+Na), cacld. 613.26, found 613.40.

### Example 14. Synthesis of 3,3′-(1,2-bis(3-(2,5-dioxo-2,5-dihydro-1H-pyrrol-1-yl)propanoyl)-hydrazine-1,2-diyl)dipropanoic Acid

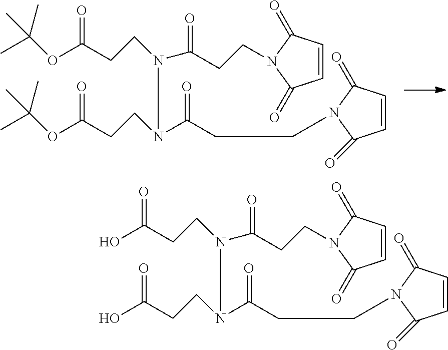

Compound 14 (700 mg, 1.18 mmol) in dioxane (4 ml) was added HCl (conc. 1 ml). The mixture was stirred for 30 min, diluted with EtOH (10 mL) and toluene (10 ml), evaporated and coevaporated with EtOH (10 ml) and toluene (10 ml) to afford the crude title product (560 mg) for next step without further purification. ESI MS m/z− C₂₀H₂₁N₄O₁₀ (M−H), cacld. 477.13, found 477.20.

### Example 15. Synthesis of Bis(2,5-dioxopyrrolidin-1-yl)-3,3′-(1,2-bis(3-(2,5-dioxo-2,5-dihydro-1H-pyrrol-1-yl)propanoyl)hydrazine-1,2-diyl)dipropanoate

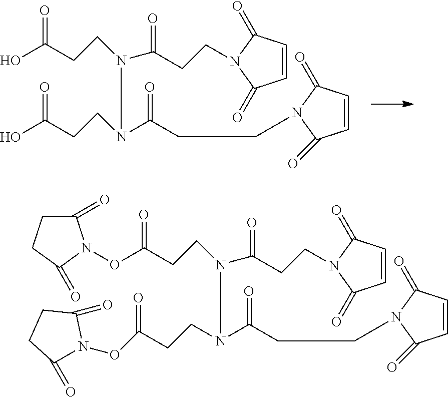 [SMILES: O=C(O)CCN(C(=O)CCN1C(=O)C=CC1=O)N(CCC(=O)O)C(=O)CCN1C(=O)C=CC1=O ; O=C(CCN(C(=O)CCN1C(=O)C=CC1=O)N(CCC(=O)ON1C(=O)CCC1=O)C(=O)CCN1C(=O)C=CC1=O)ON1C(=O)CCC1=O]

To the crude compound 3,3′-(1,2-bis(3-(2,5-dioxo-2,5-dihydro-1H-pyrrol-1-yl)propanoyl)-hydrazine-1,2-diyl)dipropanoic acid (˜560 mg, ˜1.17 mmol) in DMA (8 ml) was added NHS (400 mg, 3.47 mmol) and EDC (1.01 g, 5.26 mmol). The mixture was stirred for overnight, evaporated, concentrated and purified on SiO₂ column eluted with EtOAc/DCM (1:12˜1:7) to afford the title compound (520 mg, 65% yield in 2 steps). ESI MS m/z+ C₂₈H₂₈N₆NaO₁₄ (M+Na), cacld. 695.17, found 695.40.

### Example 16. Synthesis of Tert-Butyl 3-(2-(2-(2-hydroxyethoxy)ethoxy)ethoxy)propanoate

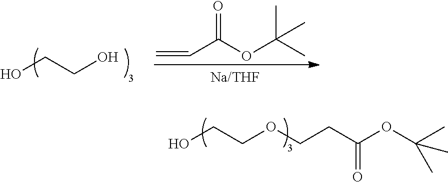

To 350 mL of anhydrous THF was added 80 mg (0.0025 mol) of sodium metal and triethylene glycol 150.1 g, 1.00 mol) with stirring. After the sodium had completely dissolved, tert-butyl acrylate (24 mL, 0.33 mol) was added. The solution was stirred for 20 h at room temperature and neutralized with 8 mL of 1.0 M HCl. The solvent was removed in vacuo and the residue was suspended in brine (250 mL) and extracted with ethyl acetate (3×125 mL). The combined organic layers were washed with brine (100 mL) then water (100 mL), dried over sodium sulfate, and the solvent was removed. The resulting colorless oil was dried under vacuum to give 69.78 g (76% yields) of the title product. ¹H NMR: 1.41 (s, 9H), 2.49 (t, 2H, J=6.4 Hz), 3.59-3.72 (m, 14H). ESI MS m/z− C₁₃H₂₅O₆ (M−H), cacld. 277.17, found 277.20.

### Example 17. Synthesis of Tert-Butyl 3-(2-(2-(2-(tosyloxy)ethoxy)ethoxy)ethoxy)propanoate

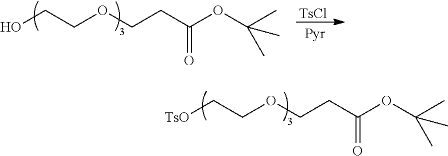

A solution of tert-Butyl 3-(2-(2-(2-hydroxyethoxy)ethoxy)ethoxy)propanoate (10.0 g, 35.95 mmol) in acetonitrile (50.0 mL) was treated with pyridine (20.0 mL). A solution of tosyl chloride (7.12 g, 37.3 mmol) in 50 mL acetonitrile was added dropwise via an addition funnel over 30 minutes. After 5 h TLC analysis revealed that the reaction was complete. The pyridine hydrochloride that had formed was filtered off and the solvent was removed. The residue was purified on silica gel by eluting from with 20% ethyl acetate in hexane to with neat ethyl acetate to give 11.2 g (76% yield) of the title compound. ¹H NMR: 1.40 (s, 9H), 2.40 (s, 3H), 2.45 (t, 2H, J=6.4 Hz), 3.52-3.68 (m, 14H), 4.11 (t, 2H, J=4.8 Hz), 7.30 (d, 2H, J=8.0 Hz), 7.75 (d, 2H, J=8.0 Hz); ESI MS m/z+ C₂₀H₃₃O₈S (M+H), cacld. 433.18, found 433.30.

### Example 18. Synthesis of Tert-Butyl 3-(2-(2-(2-azidoethoxy)ethoxy)ethoxy)propanoate

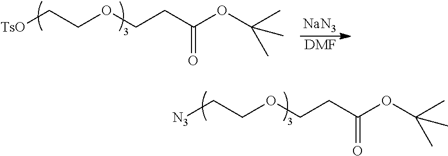

To 50 mL of DMF was added tert-butyl 3-(2-(2-(2-(tosyloxy)ethoxy)ethoxy)ethoxy)-propanoate (4.0 g, 9.25 mmol) and sodium azide (0.737 g, 11.3 mmol) with stirring. The reaction was heated to 80° C. After 4 h TLC analysis revealed that the reaction was complete. The reaction was cooled to room temperature and quenched with water (25 mL). The aqueous layer was separated and extracted into ethyl acetate (3×35 mL). The combined organic layers were dried over anhydrous magnesium sulfate, filtered, and the solvent removed in vacuo. The crude azide product (2.24 g, 98% yield, about 93% pure by HPLC) was used for next step without further purification. ¹H NMR (CDCl₃): 1.40 (s, 9H), 2.45 (t, 2H, J=6.4 Hz), 3.33 (t, 2H, J=5.2 Hz), 3.53-3.66 (m, 12H). ESI MS m/z+ C₁₃H₂₆N₃O₈ (M+H), cacld. 304.18, found 304.20.

### Example 19. Synthesis of 3-(2-(2-(2-azidoethoxy)ethoxy)ethoxy)propanoic Acid

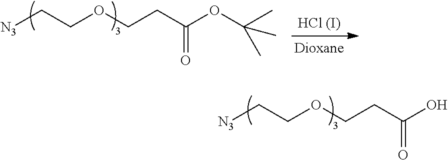

Tert-butyl 3-(2-(2-(2-azidoethoxy)ethoxy)ethoxy)propanoate (2.20 g, 7.25 mmol) in 1,4-dioxane (40 ml) was added HCl (12 M, 10 ml). The mixture was stirred for 40 min, diluted with dioxane (20 ml) and toluene (40 ml), evaporated and co-evaporated with dioxane (20 ml) and toluene (40 ml) to dryness to afford the crude title product for the next step without further production (1.88 g, 105% yield, ˜92% pure by HPLC). MS ESI m/z calcd for C₉H₁₈N₃O₅ [M+H]⁺ 248.12, found 248.40.

### Example 20. Synthesis of 13-Amino-4,7,10-trioxadodecanoic Acid Tert-butyl Ester, and 13-Amino-bis(4,7,10-trioxadodecanoic Acid Tert-Butyl Ester)

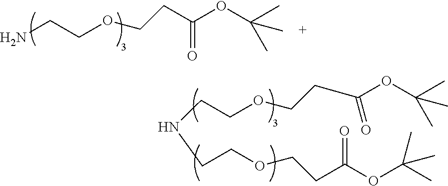

The crude azide material 3-(2-(2-(2-azidoethoxy)ethoxy)ethoxy)propanoic acid (5.0 g, ˜14.84 mmol) was dissolved in ethanol (80 mL) and 300 mg of 10% Pd/C was added. The system was evacuated under vacuum and placed under 2 atm of hydrogen gas via hydrogenation reactor with vigorous stirring. The reaction was then stirred overnight at room temperature and TLC showed that the starting materials disappeared. The crude reaction was passed through a short pad of Celite rinsing with ethanol. The solvent was removed and the amine purified on silica gel using a mixture of methanol (from 5% to 15%) and 1% triethylamine in methylene chloride as the eluant to give 13-amino-4,7,10-trioxadodecanoic acid tert-butyl ester (1.83 g, 44% yield, ESI MS m/z+ C₁₃H₂₇NO₅ (M+H), cacld. 278.19, found 278.30) and 13-amino-bis(4,7,10-trioxadodecanoic acid tert-butyl ester) (2.58 g, 32% yield, ESI MS m/z+ C₂₆H₅₂NO₁₀ (M+H), cacld. 538.35, found 538.40).

### Example 21. Synthesis of 3-(2-(2-(2-Aminoethoxy)ethoxy)ethoxy)propanoic Acid, HCl Salt

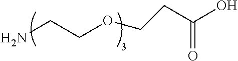

To 13-amino-4,7,10-trioxadodecanoic acid tert-butyl ester (0.80 g, 2.89 mmol) in 30 mL of dioxane was 10 ml of HCl (36%) with stirring. After 0.5 h TLC analysis revealed that the reaction was complete, the reaction mixture was evaporated, and co-evaporated with EtOH and EtOH/Toluene to form the title product in HCl salt (>90% pure, 0.640 g, 86% yield) without further purification. ESI MS m/z+ C₉H₂₀NO₅ (M+H), cacld. 222.12, found 222.20.

### Example 22. 13-Amino-bis(4,7,10-trioxadodecanoic Acid, HCl Salt

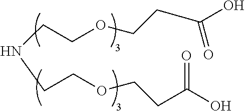

To 13-amino-bis(4,7,10-trioxadodecanoic acid tert-butyl ester) (1.00 g, 1.85 mmol) in 30 mL of dioxane was 10 ml of HCl (36%) with stirring. After 0.5 h TLC analysis revealed that the reaction was complete, the reaction mixture was evaporated, and co-evaporated with EtOH and EtOH/Toluene to form the title product in HCl salt (>90% pure, 0.71 g, 91% yield) without further purification. ESI MS m/z+ C₁₈H₃₆NO₁₀ (M+H), cacld. 426.22, found 426.20.

### Example 23. Synthesis of Tert-butyl 3-(2-(2-(2-hydroxyethoxy)ethoxy)ethoxy) propanoate

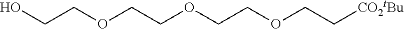 [SMILES: CC(C)(C)OC(=O)CCOCCOCCOCCO]

To a solution of 2,2′-(ethane-1,2-diylbis(oxy))diethanol (55.0 mL, 410.75 mmol, 3.0 eq.) in anhydrous THE (200 mL) was added sodium (0.1 g). The mixture was stirred until Na disappeared and then tert-butyl acrylate (20.0 mL, 137.79 mmol, 1.0 eq.) was added dropwise. The mixture was stirred overnight and then quenched by HCl solution (20.0 mL, 1N) at 0° C. THE was removed by rotary evaporation, brine (300 mL) was added and the resulting mixture was extracted with EtOAc (3×100 mL). The organic layers were washed with brine (3×300 mL), dried over anhydrous Na₂SO₄, filtered and concentrated to afford a colourless oil (30.20 g, 79.0% yield), which was used without further purification. MS ESI m/z calcd for C₁₃H₂₇O₆ [M+H]⁺ 278.1729, found 278.1730.

### Example 24. Synthesis of Tert-butyl 3-(2-(2-(2-(tosyloxy)ethoxy)ethoxy)ethoxy) Propanoate

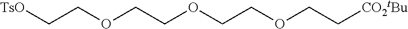 [SMILES: Cc1ccc(S(=O)(=O)OCCOCCOCCOCCC(=O)OC(C)(C)C)cc1]

To a solution of tert-butyl 3-(2-(2-(2-hydroxyethoxy)ethoxy)ethoxy) propanoate (30.20 g, 108.5 mmol, 1.0 eq.) and TsCl (41.37 g, 217.0 mmol, 2.0 eq.) in anhydrous DCM (220 mL) at 0° C. was added TEA (30.0 mL, 217.0 mmol, 2.0 eq.). The mixture was stirred at room temperature overnight, and then washed with water (3×300 mL) and brine (300 mL), dried over anhydrous Na₂SO₄, filtered, concentrated and purified by SiO₂ column chromatography (3:1 hexanes/EtOAc) to give a colourless oil (39.4 g, 84.0% yield). MS ESI m/z calcd for C₂₀H₃₃O₈S [M+H]⁺ 433.1818, found 433.2838.

### Example 25. Synthesis of Tert-butyl 3-(2-(2-(2-azidoethoxy)ethoxy)ethoxy) Propanoate

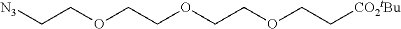 [SMILES: CC(C)(C)OC(=O)CCOCCOCCOCCN=[N+]=[N-]]

To a solution of tert-butyl 3-(2-(2-(2-(tosyloxy)ethoxy)ethoxy)ethoxy) propanoate (39.4 g, 91.1 mmol, 1.0 eq.) in anhydrous DMF (100 mL) was added NaN₃ (20.67 g, 316.6 mmol, 3.5 eq.). The mixture was stirred at room temperature overnight. Water (500 mL) was added and extracted with EtOAc (3×300 mL). The combined organic layers were washed with water (3×900 mL) and brine (900 mL), dried over anhydrous Na₂SO₄, filtered, concentrated and purified by SiO₂ column chromatography (5:1 hexanes/EtOAc) to give a light yellow oil (23.8 g, 85.53% yield). MS ESI m/z calcd for C₁₃H₂₅O₃N₅Na [M+Na]⁺ 326.2, found 326.2.

### Example 26. Synthesis of Tert-butyl 3-(2-(2-(2-aminoethoxy)ethoxy)ethoxy) Propanoate

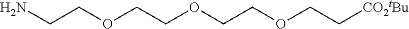 [SMILES: CC(C)(C)OC(=O)CCOCCOCCOCCN]

Raney-Ni (7.5 g, suspended in water) was washed with water (three times) and isopropyl alcohol (three times) and mixed with tert-butyl 3-(2-(2-(2-azidoethoxy)ethoxy)ethoxy) propanoate (5.0 g, 16.5 mmol) in isopropyl alcohol. The mixture was stirred under a H2 balloon at r.t. for 16 h and then filtered over a Celite pad, with washing of the pad with isopropyl alcohol. The filtrate was concentrated and purified by column chromatography (5-25% MeOH/DCM) to give a light yellow oil (2.60 g, 57% yield). MS ESI m/z calcd for C₁₃H₂₈NO₅ [M+H]⁺ 279.19; found 279.19.

### Example 27. Synthesis of 2-(2-(dibenzylamino)ethoxy)ethanol

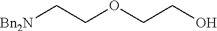

2-(2-aminoethoxy)ethanol (21.00 g, 200 mmol, 1.0 eq.) and K₂CO₃ (83.00 g, 600 mmol, 3.0 eq.) in acetonitrile (350 mL) was added BnBr (57.0 mL, 480 mmol, 2.4 eq.). The mixture was refluxed overnight. Water (1 L) was added and extracted with EtOAc (3×300 mL). The combined organic layers were washed with brine (1000 mL), dried over anhydrous Na₂SO₄, filtered, concentrated and purified by SiO₂ column chromatography (4:1 hexanes/EtOAc) to give a colourless oil (50.97 g, 89.2% yield). MS ESI m/z calcd for C₁₈H₂₃NO₂Na [M+Na]⁺ 309.1729, found 309.1967.

### Example 28. Synthesis of Tert-butyl 3-(2-(2-(dibenzylamino)ethoxy)ethoxy) Propanoate

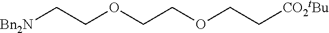

To a mixture of 2-(2-(dibenzylamino)ethoxy)ethanol (47.17 g, 165.3 mmol, 1.0 eq.), tert-butyl acrylate (72.0 mL, 495.9 mmol, 3.0 eq.) and n-Bu₄NI (6.10 g, 16.53 mmol, 0.1 eq.) in DCM (560 mL) was added sodium hydroxide solution (300 mL, 50%). The mixture was stirred overnight. The organic layer was separated and the water layer was extracted with EtOAc (3×100 mL). The organic layers were washed with water (3×300 mL) and brine (300 mL), dried over anhydrous Na₂SO₄, filtered, concentrated and purified by SiO₂ column chromatography (7:1 hexanes/EtOAc) to give a colourless oil (61.08 g, 89.4% yield). MS ESI m/z calcd for C₂₅H₃₆NO₄ 4 [M+H]⁺ 414.2566, found 414.2384.

### Example 29. Synthesis of Tert-butyl 3-(2-(2-aminoethoxy)ethoxy)propanoate

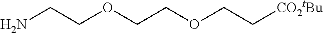 [SMILES: CC(C)(C)OC(=O)CCOCCOCCN]

To a solution of tert-butyl 3-(2-(2-(dibenzylamino)ethoxy)ethoxy) propanoate (20.00 g, 48.36 mmol, 1.0 eq.) in THE (30 mL) and MeOH (60 mL) was added Pd/C (2.00 g, 10 wt %, 50% wet) in a hydrogenation bottle. The mixture was shaken at 1 atom pressure H2 overnight, filtered through Celite (filter aid), and the filtrate was concentrated to afford a colourless oil (10.58 g, 93.8% yield). MS ESI m/z calcd for C₁₁H₂₄NO₄ [M+H]⁺ 234.1627, found 234.1810.

### Example 30. Synthesis of Tert-butyl 3-(2-(2-hydroxyethoxy)ethoxy)propanoate

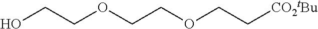 [SMILES: CC(C)(C)OC(=O)CCOCCOCCO]

To a solution of 2,2′-oxydiethanol (19.7 mL, 206.7 mmol, 3.0 eq.) in anhydrous THE (100 mL) was added sodium (0.1 g). The mixture was stirred until Na disappeared and then tert-butyl acrylate (10.0 mL, 68.9 mmol, 1.0 eq.) was added dropwise. The mixture was stirred overnight, and brine (200 mL) was added and extracted with EtOAc (3×100 mL). The organic layers were washed with brine (3×300 mL), dried over anhydrous Na₂SO₄, filtered, concentrated and purified by SiO₂ column chromatography (1:1 hexanes/EtOAc) to give to a colourless oil (8.10 g, 49.4% yield). MS ESI m/z calcd for C₁₁H₂₃O₅ [M+H]⁺ 235.1467, found 235.1667.

### Example 31. Synthesis of Tert-butyl 3-(2-(2-(tosyloxy)ethoxy)ethoxy)propanoate

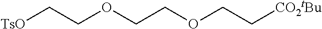 [SMILES: Cc1ccc(S(=O)(=O)OCCOCCOCCC(=O)OC(C)(C)C)cc1]

To a solution of tert-butyl 3-(2-(2-hydroxyethoxy)ethoxy)propanoate (6.24 g, 26.63 mmol, 1.0 eq.) and TsCl (10.15 g, 53.27 mmol, 2.0 eq.) in anhydrous DCM (50 mL) at 0° C. was added pyridine (4.3 mL, 53.27 mmol, 2.0 eq.). The mixture was stirred at room temperature overnight, and then washed with water (100 mL) and the water layer was extracted with DCM (3×50 mL). The combined organic layers were washed with brine (300 mL), dried over anhydrous Na₂SO₄, filtered, concentrated and purified by SiO₂ column chromatography (5:1 hexanes/EtOAc) to give a colourless oil (6.33 g, 61.3% yield). MS ESI m/z calcd for C₁₈H₂₇O₇S [M+H]⁺ 389.1556, found 389.2809.

### Example 32. Synthesis of Tert-butyl 3(2-(2-azidoethoxy)ethoxy)propanoate

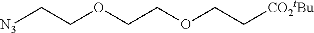 [SMILES: CC(C)(C)OC(=O)CCOCCOCCN=[N+]=[N-]]

To a solution of tert-butyl 3-(2-(2-(tosyloxy)ethoxy)ethoxy)propanoate (5.80 g, 14.93 mmol, 1.0 eq.) in anhydrous DMF (20 mL) was added NaN₃ (5.02 g, 77.22 mmol, 5.0 eq.). The mixture was stirred at room temperature overnight. Water (120 mL) was added and extracted with EtOAc (3×50 mL). The combined organic layers were washed with water (3×150 mL) and brine (150 mL), dried over anhydrous Na₂SO₄, filtered, concentrated and purified by SiO₂ column chromatography (5:1 hexanes/EtOAc) to give a colourless oil (3.73 g, 69.6% yield). MS ESI m/z calcd for C₁₁H₂₂O₃N₄Na[M+H]⁺ 260.1532, found 260.2259.

### Example 33. Synthesis of Tert-butyl 3-(2-(2-aminoethoxy)ethoxy)propanoate

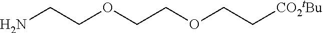 [SMILES: CC(C)(C)OC(=O)CCOCCOCCN]

tert-Butyl 3-(2-(2-azidoethoxy)ethoxy)propanoate (0.18 g, 0.69 mmol) was dissolved in MeOH (3.0 mL, with 60 μL concentrated HCl) and hydrogenated with Pd/C (10 wt %, 20 mg) under a H2 balloon for 30 min. The catalyst was filtered through a Celite pad, with washing of the pad with MeOH. The filtrate was concentrated to give a colorless oil (0.15 g, 93% yield). MS ESI m/z calcd for C₁₁H₂₄NO₄ [M+H]⁺ 234.16; found 234.14.

### Example 34. Synthesis of 3-(2-(2-azidoethoxy)ethoxy)propanoic Acid

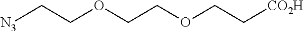 [SMILES: [N-]=[N+]=NCCOCCOCCC(=O)O]

tert-Butyl 3-(2-(2-azidoethoxy)ethoxy)propanoate (2.51 g, 9.68 mmol) dissolved in 1,4-dioxane (30 mL) was treated with 10 ml of HCl (conc.) at r.t. The mixture was stirred for 35 min, diluted with EtOH (30 ml) and toluene (30 ml) and concentrated under vacuum. The crude mixture was purified on silica gel using a mixture of methanol (from 5% to 10%) and 1% formic acid in methylene chloride as the eluant to give title compound (1.63 g, 83% yield), ESI MS m/z C₇H₁₂N₃O₄ [M−H]⁻, cacld. 202.06, found 202.30.

### Example 35. Synthesis of 2,5-dioxopyrrolidin-1-yl 3-(2-(2-azidoethoxy)ethoxy)propanoate

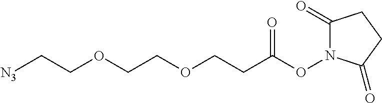 [SMILES: [N-]=[N+]=NCCOCCOCCC(=O)ON1C(=O)CCC1=O]

To 3-(2-(2-azidoethoxy)ethoxy)propanoic acid (1.60 g, 7.87 mmol) in 30 mL of dichloromethane was added NHS (1.08 g, 9.39 mmol) and EDC (3.60 g, 18.75 mmol) with stirring. After 8 h TLC analysis revealed that the reaction was complete, the reaction mixture was concentrated and purified on silica gel using a mixture of ethyl acetate (from 5% to 10%) in methylene chloride as the eluant to give title compound (1.93 g, 82% yield). ESI MS m/z C₁₁H₁₇N₄O₆ [M+H]⁺, cacld. 301.11, found 301.20.

### Example 36. Synthesis of 2,5-dioxopyrrolidin-1-yl 3-(2-(2-(2-azidoethoxy)ethoxy)ethoxy)propanoate

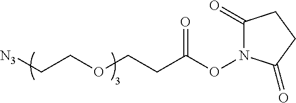

To 3-(2-(2-(2-azidoethoxy)ethoxy)ethoxy)propanoic acid (4.50 g, 18.21 mmol) in 80 mL of dichloromethane was added NHS (3.0 g, 26.08 mmol) and EDC (7.60 g, 39.58 mmol) with stirring. After 8 h TLC analysis revealed that the reaction was complete, the reaction mixture was concentrated and purified on silica gel using a mixture of ethyl acetate (from 5% to 10%) in methylene chloride as the eluant to give title compound (5.38 g, 86% yield). ESI MS m/z C₁₃H₂₀N₄O₇ [M+H]⁺, cacld. 345.13, found 345.30.

### Example 37. Synthesis of (14S,17S)-1-azido-17-(2-(tert-butoxy)-2-oxoethyl)-14-(4-((tert-butoxycarbonyl)-amino)butyl)-12,15-dioxo-3,6,9-trioxa-13,16-diazaoctadecan-18-oic Acid

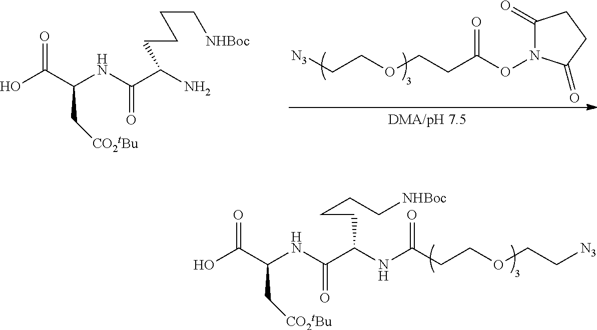

To a solution of (S)-2-((S)-2-amino-6-((tert-butoxycarbonyl)amino)hexanamido)-4-(tert-butoxy)-4-oxobutanoic acid (2.81 g, 6.73 mmol) in the mixture of DMA (70 ml) and 0.1 M NaH₂PO₄ (50 ml, pH 7.5) was added 2,5-dioxopyrrolidin-1-yl 3-(2-(2-(2-azidoethoxy)ethoxy)-ethoxy)propanoate (3.50 g, 10.17). The mixture was stirred for 4 h, evaporated in vacuo, purified on silica gel using a mixture of methanol (from 5% to 15%) in methylene chloride containing 0.5% acetic acid as the eluant to give title compound (3.35 g, 77% yield). ESI MS m/z C₂₈H₅₁N₆O₁₁ [M+H]⁺, cacld. 647.35, found 647.80.

### Example 38. Synthesis of (14S,17S)-tert-butyl 1-azido-14-(4-((tert-butoxycarbonyl)-amino)butyl)-17-((4-(hydroxymethyl)phenyl)carbamoyl)-12,15-dioxo-3,6,9-trioxa-13,16-diazanonadecan-19-oate

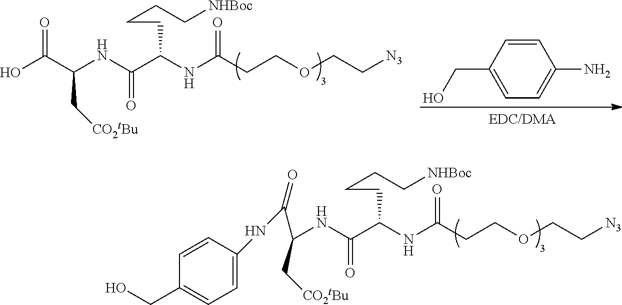

(14S,17S)-1-azido-17-(2-(tert-butoxy)-2-oxoethyl)-14-(4-((tert-butoxycarbonyl)-amino)butyl)-12,15-dioxo-3,6,9-trioxa-13,16-diazaoctadecan-18-oic acid (3.30 g, 5.10 mmol) and (4-aminophenyl)methanol (0.75 g, 6.09) in DMA (25 ml) was added EDC (2.30 g, 11.97 mmol). The mixture was stirred for overnight, evaporated in vacuo, purified on silica gel using a mixture of methanol (from 5% to 8%) in methylene chloride containing as the eluant to give title compound (3.18 g, 83% yield). ESI MS m/z C₃₅H₅₈N₇O₁₁ [M+H]⁺, cacld. 752.41, found 752.85.

### Example 39. Synthesis of (14S,17S)-tert-butyl 1-amino-4-(4-((tert-butoxycarbonyl)amino)-butyl)-17-((4-(hydroxymethyl)phenyl)carbamoyl)-12,15-dioxo-3,6,9-trioxa-13,16-diazanonadecan-19-oate

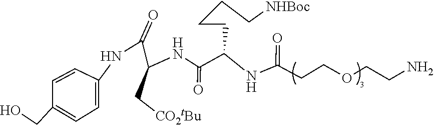

To a solution of (14S,17S)-tert-butyl 1-azido-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate (1.50 g, 1.99 mmol) in THE (35 mL) was added Pd/C (200 mg, 10% Pd, 50% wet) in a hydrogenation bottle. The mixture was shaken at 1 atom pressure H₂ overnight, filtered through Celite (filter aid), and the filtrate was concentrated to afford the title compound (1.43 g, 99% yield) which was used immediately for the next step without further purification. ESI MS m/z C₃₅H₆₀N₅O₁₁ [M+H]⁺, cacld. 726.42, found 726.70.

### Example 40. Synthesis of (S)-15-azido-5-isopropyl-4,7-dioxo-10,13-dioxa-3,6-diazapentadecan-1-oic Acid

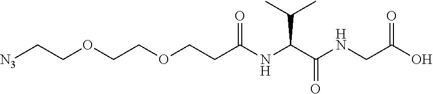 [SMILES: CC(C)[C@H](NC(=O)CCOCCOCCN=[N+]=[N-])C(=O)NCC(=O)O]

To a solution of (S)-2-(2-amino-3-methylbutanamido)acetic acid (Val-Gly) (1.01 g, 5.80 mmol) in the mixture of DMA (50 ml) and 0.1 M NaH₂PO₄ (50 ml, pH 7.5) was added 2,5-dioxopyrrolidin-1-yl 3-(2-(2-azidoethoxy)ethoxy)propanoate (1.90 g, 6.33). The mixture was stirred for 4 h, evaporated in vacuo, purified on silica gel using a mixture of methanol (from 5% to 15%) in methylene chloride containing 0.5% acetic acid as the eluant to give title compound (1.52 g, 73% yield). ESI MS m/z C₁₄H₂₆N₅O₆ [M+H]⁺, cacld. 360.18, found 360.40.

### Example 41. Synthesis of (S)-2,5-dioxopyrrolidin-1-yl 15-azido-5-isopropyl-4,7-dioxo-10,13-dioxa-3,6-diazapentadecan-1-oate

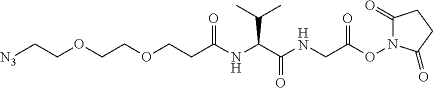 [SMILES: CC(C)[C@H](NC(=O)CCOCCOCCN=[N+]=[N-])C(=O)NCC(=O)ON1C(=O)CCC1=O]

To a solution of (S)-15-azido-5-isopropyl-4,7-dioxo-10,13-dioxa-3,6-diazapentadecan-1-oic acid (1.50 g, 4.17 mmol) in 40 mL of dichloromethane was added NHS (0.88 g, 7.65 mmol) and EDC (2.60 g, 13.54 mmol) with stirring. After 8 h TLC analysis revealed that the reaction was complete, the reaction mixture was concentrated and purified on silica gel using a mixture of ethyl acetate (from 5% to 20%) in methylene chloride as the eluant to give title compound (1.48 g, 78% yield). ESI MS m/z C₁₈H₂₉N₆O₈ [M+H]⁺, cacld. 457.20, found 457.50.

### Example 42. Synthesis of 4-(((benzyloxy)carbonyl)amino)butanoic Acid

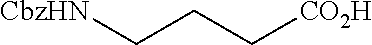 [SMILES: O=C(O)CCCNC(=O)OCc1ccccc1]

A solution of 4-aminobutyric acid (7.5 g, 75 mmol) and NaOH (6 g, 150 mmol) in H₂O (40 mL) was cooled to 0° C. and treated with a solution of CbzCl (16.1 g, 95 mmol) in THE (32 ml) dropwise. After 1 h, the reaction was allowed to warm to r.t. and stirred for 3 h. THE was removed under vacuum, the pH of the aqueous solution was adjusted to 1.5 by addition of 6 N HCl. Extracted with ethyl acetate, and the organic layer was washed with brine, dried and concentrated to give the title compound (16.4 g, 92% yield). MS ESI m/z calcd for C₁₂H₁₆NO₅ [M+H]⁺ 238.10, found 238.08.

### Example 43. Synthesis of Tert-butyl 4-(((benzyloxy)carbonyl)amino)butanoate

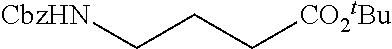 [SMILES: CC(C)(C)OC(=O)CCCNC(=O)OCc1ccccc1]

DMAP (0.8 g, 6.56 mmol) and DCC (17.1 g, 83 mmol) were added to a solution of 4-(((benzyloxy)carbonyl)amino)butanoic acid (16.4 g, 69.2 mmol) and t-BuOH (15.4 g, 208 mmol) in DCM (100 mL). After stirring at r.t. overnight, the reaction was filtered and filtrate concentrated. The residue was dissolved in ethyl acetate and the washed with 1N HCl, brine and dried over Na₂SO₄. Concentration and purification by column chromatography (10 to 50% EtOAc/hexanes) yielded the title compound (7.5 g, 37% yield). MS ESI m/z calcd for C₁₆H₂₃NO₄Na [M+Na]⁺316.16, found 316.13.

### Example 44. Synthesis of Tert-butyl 4-aminobutanoate

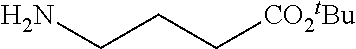 [SMILES: CC(C)(C)OC(=O)CCCN]

tert-Butyl 4-(((benzyloxy)carbonyl)amino)butanoate (560 mg, 1.91 mmol) was dissolved in MeOH (50 mL), and mixed with Pd/C catalyst (10 wt %, 100 mg) then hydrogenated (1 atm) at room temperature for 3 h. The catalyst was filtered off and all volatiles were removed under vacuum to afford the title compound (272 mg, 90% yield). MS ESI m/z calcd for C₈H₁₈NO₂ [M+H]⁺ 160.13, found 160.13.

### Example 45. Synthesis of di-tert-butyl 3,3′-(benzylazanediyl)dipropanoate

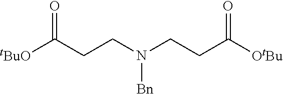 [SMILES: CC(C)(C)OC(=O)CCN(CCC(=O)OC(C)(C)C)Cc1ccccc1]

A mixture of phenylmethanamine (2.0 mL, 18.29 mmol, 1.0 eq) and tert-butyl acrylate (13.3 mL, 91.46 mmol, 5.0 eq) was refluxed at 80° C. overnight and then concentrated. The crude product was purified by SiO₂ column chromatography (20:1 hexanes/EtOAc) to give the title compound as colourless oil (5.10 g, 77% yield). ESI MS m/z: calcd for C₂₁H₃₄NO₄ [M+H]⁺ 364.2, found 364.2. ¹H NMR (400 MHz, CDCl₃) δ 7.38-7.21 (m, 5H), 3.58 (s, 2H), 2.76 (t, J=7.0 Hz, 4H), 2.38 (t, J=7.0 Hz, 4H), 1.43 (s, 17H).

### Example 46. Synthesis of di-tert-butyl 3,3′-azanediyldipropanoate

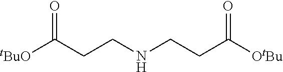 [SMILES: CC(C)(C)OC(=O)CCNCCC(=O)OC(C)(C)C]

To a solution of di-tert-butyl 3,3′-(benzylazanediyl)dipropanoate (1.37 g, 3.77 mmol, 1.0 equiv) in MeOH (10 mL) was added Pd/C (0.20 g, 10% Pd/C, 50% wet) in a hydrogenation bottle. The mixture was shaken overnight under H2 atmosphere and then filtered through a Celite pad. The filtrate was concentrated to give the title compound as colourless oil (1.22 g, 89% yield). ESI MS m/z: calcd for C₁₄H₂₈NO₄ [M+H]⁺ 274.19, found 274.20.

### Example 47. Synthesis of Tert-butyl 4-(2-(((benzyloxy)carbonyl)amino)propan amido)-butanoate

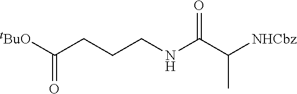 [SMILES: CC(NC(=O)OCc1ccccc1)C(=O)NCCCC(=O)OC(C)(C)C]

To a solution of tert-butyl 4-aminobutanoate (1.00 g, 6.28 mmol, 1.0 eq.) and Z-L-alaine (2.10 g, 9.42 mmol, 1.5 eq.) in anhydrous DCM (50 mL) at 0° C. were added HATU (3.10 g, 8.164 mmol, 1.3 eq.) and TEA (2.6 mL, 18.8 mmol, 3.0 eq.). The reaction was stirred at 0° C. for 10 min., then warmed to room temperature and stirred overnight. The mixture was diluted with DCM and washed with water and brine, dried over anhydrous Na₂SO₄, concentrated and purified by SiO₂ column chromatography (10:3 petroleum ether/ethyl acetate) to give the title compound as a colorless oil (1.39 g, 61% yield). ESI MS m/z: calcd for C₁₉H₂₉N₂O₅Na [M+H]⁺ 387.2, found 387.2.

### Example 48. Synthesis of Tert-butyl 4-(2-aminopropanamido)butanoate

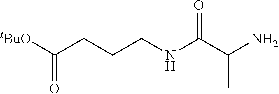 [SMILES: CC(N)C(=O)NCCCC(=O)OC(C)(C)C]

To a solution of tert-butyl 4-(2-(((benzyloxy)carbonyl)amino)propanamido) butanoate (1.39 g, 3.808 mmol, 1.0 eq.) in MeOH (12 mL) was added Pd/C (0.20 g, 10 wt %, 10% wet) in a hydrogenation bottle. The mixture was shaken for 2 h and then filtered through Celite (filter aid), concentrated to give the title compound as a light yellow oil (0.838 g, 95% yield). ESI MS m/z: calcd. for C₁₁H₂₃N₂O₃ [M+H]⁺ 231.16, found 231.15.

### Example 49. Synthesis of 3-(2-(2-(dibenzylamino)ethoxy)ethoxy)propanoic Acid

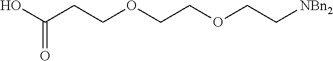

To a solution of tert-butyl 3-(2-(2-(dibenzylamino)ethoxy)ethoxy)propanoate (2.3 g, 5.59 mmol, 1.0 eq) in DCM (10 mL) at room temperature was added TFA (5 mL). After stirring for 90 min., the reaction mixture was diluted with anhydrous toluene and concentrated, this operation was repeated for three times to give the title compound as a light yellow oil (2.0 g, theoretical yield), which was directly used in the next step. ESI MS m/z calcd. for C₂₁H₂₈NO₄ [M+H]⁺358.19, found 358.19.

### Example 50. Synthesis of perfluorophenyl 3-(2-(2-(dibenzylamino)ethoxy) Ethoxy)-propanoate

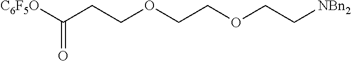

To a solution of 3-(2-(2-(dibenzylamino)ethoxy)ethoxy)propanoic acid (2.00 g, 5.59 mmol, 1.0 eq.) in anhydrous DCM (30 mL) at 0° C. was added DIPEA until pH was neutral, and then PFP (1.54 g, 8.38 mmol, 1.5 eq.) and DIC (1.04 mL, 6.70 mmol, 1.2 eq.) were added. After 10 min. the reaction was warmed to room temperature and stirred overnight. The mixture was filtered, concentrated and purified by SiO₂ column chromatography (15:1 petroleum ether/ethyl acetate) to give the title compound as colourless oil (2.10 g, 72% yield). ESI MS m/z: calcd. for C₂₇H₂₇F₅NO₄ [M+H]⁺ 524.2, found 524.2.

### Example 51. Synthesis of Tert-butyl 2-benzyl-13-methyl-11,14-dioxo-1-phenyl-5,8-dioxa-2,12,15-triazanonadecan-19-oate

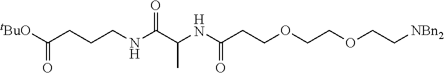

To a solution of tert-butyl 4-(2-aminopropanamido)butanoate (0.736 g, 3.2 mmol, 1.0 eq.) and perfluorophenyl 3-(2-(2-(dibenzylamino)ethoxy) ethoxy)propanoate (2.01 g, 3.84 mmol, 1.2 eq.) in anhydrous DMA (20 mL) at 0° C. was added DIPEA (1.7 mL, 9.6 mmol, 3.0 eq.). After stirring at 0° C. for 10 min. the reaction was warmed to room temperature and stirred overnight. Water (100 mL) was added and the mixture was extracted with EtOAc (3×100 mL). The combined organic layers were washed with water (3×200 mL) and brine (200 mL), dried over Na₂SO₄, filtered, concentrated and purified by SiO₂ column chromatography (25:2 DCM/MeOH) to give the title compound as a colourless oil (1.46 g, 80% yield). ESI MS m/z: calcd. for C₃₂H₄₈N₃O₆ [M+H]⁺ 570.34, found 570.33.

### Example 52. Synthesis of 2-benzyl-13-methyl-11,14-dioxo-1-phenyl-5,8-dioxa-2,12,15-triazanonadecan-19-oic Acid

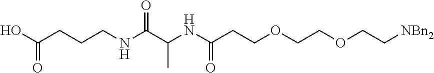

To a solution of tert-butyl 2-benzyl-13-methyl-11,14-dioxo-1-phenyl-5,8-dioxa-2,12,15-triazanonadecan-19-oate (0.057 g, 0.101 mmol, 1.0 eq) in DCM (3 mL) at room temperature was added TFA (1 mL) and stirred for 40 min. The reaction was diluted with anhydrous toluene and then concentrated. This operation was repeated three times to give the title compound as a colourless oil (0.052 g, theoretical yield), which was used directly in the next step. ESI MS m/z: calcd for C₂₈H₄₀N₃O₆ [M+H]⁺ 514.28, found 514.28.

### Example 53. Synthesis of 4-(((benzyloxy)carbonyl)amino)butanoic Acid

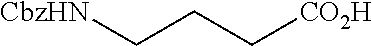 [SMILES: O=C(O)CCCNC(=O)OCc1ccccc1]

A solution of 4-aminobutyric acid (7.5 g, 75 mmol) and NaOH (6 g, 150 mmol) in H₂O (40 mL) was cooled to 0° C. and treated with a solution of CbzCl (16.1 g, 95 mmol) in THE (32 ml) dropwise. After 1 h, the reaction was allowed to warm to r.t. and stirred for 3 h. THE was removed under vacuum, the pH of the aqueous solution was adjusted to 1.5 by addition of 6 N HCl. Extracted with ethyl acetate, and the organic layer was washed with brine, dried and concentrated to give the title compound (16.4 g, 92% yield). MS ESI m/z calcd for C12H16NO5 [M+H]+ 238.10, found 238.08.

### Example 54. Synthesis of Tert-butyl 4-(((benzyloxy)carbonyl)amino)butanoate

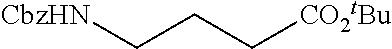 [SMILES: CC(C)(C)OC(=O)CCCNC(=O)OCc1ccccc1]

DMAP (0.8 g, 6.56 mmol) and DCC (17.1 g, 83 mmol) were added to a solution of 4-(((benzyloxy)carbonyl)amino)butanoic acid (16.4 g, 69.2 mmol) and t-BuOH (15.4 g, 208 mmol) in DCM (100 mL). After stirring at r.t. overnight, the reaction was filtered and filtrate concentrated. The residue was dissolved in ethyl acetate and the washed with 1N HCl, brine and dried over Na₂SO₄. Concentration and purification by column chromatography (10 to 50% EtOAc/hexanes) yielded the title compound (7.5 g, 37% yield). MS ESI m/z calcd for C₁₆H₂₃NO₄Na [M+Na]⁺ 316.16, found 316.13.

### Example 55. Synthesis of Tert-butyl 4-aminobutanoate

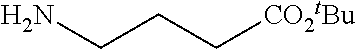 [SMILES: CC(C)(C)OC(=O)CCCN]

tert-Butyl 4-(((benzyloxy)carbonyl)amino)butanoate (560 mg, 1.91 mmol) was dissolved in MeOH (50 mL), and mixed with Pd/C catalyst (10 wt %, 100 mg) then hydrogenated (1 atm) at room temperature for 3 h. The catalyst was filtered off and all volatiles were removed under vacuum to afford the title compound (272 mg, 90% yield). MS ESI m/z calcd for C₈H₁₈NO₂ [M+H]⁺ 160.13, found 160.13.

### Example 56. Synthesis of Tert-butyl 2-(2-(((benzyloxy)carbonyl)amino)propanamido)acetate

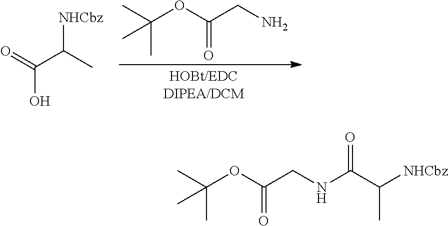

2-(((Benzyloxy)carbonyl)amino)propanoic acid (0.84 g, 5 mmol), tert-butyl 2-aminoacetate (0.66 g, 5 mmol), HOBt (0.68 g, 5 mmol), EDC (1.44 g, 7.5 mmol) were dissolved in DCM (20 ml), followed by addition of DIPEA (1.7 ml, 10 mmol). The reaction mixture was stirred at RT overnight, washed with H₂O (100 ml), and the aqueous layer was extracted with EtOAc. The organic layers were combined, dried over MgSO₄, filtered, evaporated under reduced pressure and the residue was purified on SiO₂ column to give the title product 1 (0.87 g, 52%). ESI: m/z: calcd for C₁₇H₂₅N₂O₅ [M+H]⁺: 337.17, found 337.17.

### Example 57. Synthesis of 2-(2-(((benzyloxy)carbonyl)amino)propanamido)acetic Acid

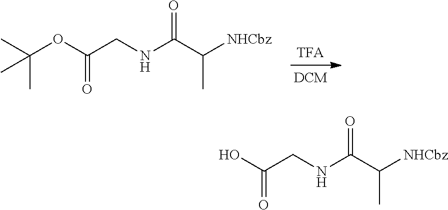 [SMILES: CC(NC(=O)OCc1ccccc1)C(=O)NCC(=O)OC(C)(C)C ; CC(NC(=O)OCc1ccccc1)C(=O)NCC(=O)O]

Tert-butyl 2-(2-(((benzyloxy)carbonyl)amino)propanamido)acetate (0.25 g, 0.74 mmol) was dissolved in DCM (30 ml), followed by addition of TFA (10 ml). The mixture was stirred at RT overnight, concentrated to afford the title compound used for the next step without further purification. ESI: m/z: calcd for C₁₃H₁₇N₂O₅ [M+H]⁺: 281.11, found 281.60.

### Example 58. Synthesis of Di-tert-butyl 14,17-dioxo-4,7,10,21,24,27-hexaoxa-13,18-diazatriacont-15-yne-1,30-dioate

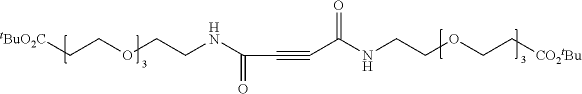

Acetylenedicarboxylic acid (0.35 g, 3.09 mmol, 1.0 eq.) was dissolved in NMP (10 mL) and cooled to 0° C., to which compound tert-butyl 3-(2-(2-(2-aminoethoxy)ethoxy)ethoxy)-propanoate (2.06 g, 7.43 mmol, 2.4 eq.) was added, followed by DMTMM (2.39 g, 8.65 mmol, 2.8 eq.) in portions. The reaction was stirred at 0° C. for 6 h and then diluted with ethyl acetate and washed with water and brine. The organic solution was concentrated and triturated with a mixture solvent of ethyl acetate and petroleum ether. The solid was filtered off and the filtrate was concentrated and purified by column chromatography (80-90% EA/PE) to give a light yellow oil (2.26 g, >100% yield), which was used without further purification. MS ESI m/z [M+H]⁺ 633.30.

### Example 59. Synthesis of 14,17-dioxo-4,7,10,21,24,27-hexaoxa-13,18-diaza Triacont-15-yne-1,30-dioic Acid

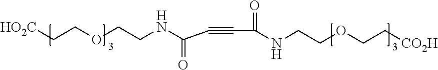

Compound di-tert-butyl 14,17-dioxo-4,7,10,21,24,27-hexaoxa-13,18-diazatriacont-15-yne-1,30-dioate (2.26 g) was dissolved in dichloromethane (15 mL) and cooled to 0° C. then treated with TFA (15 mL). The reaction was warmed to r.t. and stirred for 45 min, and then the solvent and residual TFA was removed on rotovap. The crude product was purified by column chromatography (0-15% MeOH/DCM) to give a light yellow oil (1.39 g, 86% yield for two steps). MS ESI m/z [M+H]⁺ 521.24.

### Example 60. Synthesis of Di-tert-butyl 2,5,38,41-tetramethyl-4,7,20,23,36,39-hexaoxo-10,13,16,27,30,33-hexaoxa-3,6,19,24,37,40-hexaazadotetracont-21-yne-1,42-dioate

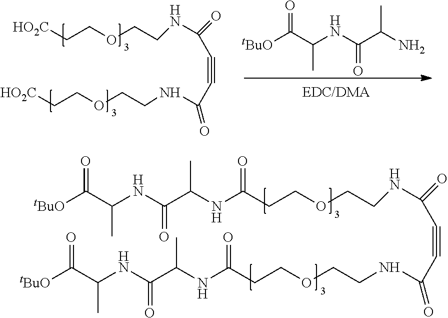

To a solution of 14,17-dioxo-4,7,10,21,24,27-hexaoxa-13,18-diaza triacont-15-yne-1,30-dioic acid (1.38 g, 2.65 mmol), tert-butyl 2-(2-aminopropanamido)propanoate (0.75 g, 3.47 mmol) in the mixture of DMA (40 ml) was added EDC (2.05 g, 10.67 mmol). The mixture was stirred for overnight, concentrated and purified on SiO₂ column eluted with EtOAc/CH₂Cl₂ (1:5 to 1:1) to afford the title compound (2.01 g, 82% yield, ˜95% pure by HPLC). MS ESI m/z calcd for C₄₂H₇₃N₆O₁₆ [M+H]⁺ 917.50, found 917.90.

### Example 61. Synthesis of 2,5,38,41-tetramethyl-4,7,20,23,36,39-hexaoxo-10,13,16,27,30,33-hexaoxa-3,6,19,24,37,40-hexaazadotetracont-21-yne-1,42-dioic Acid

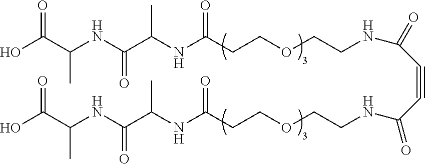

Di-di-tert-butyl 2,5,38,41-tetramethyl-4,7,20,23,36,39-hexaoxo-10,13,16,27,30,33-hexaoxa-3,6,19,24,37,40-hexaazadotetracont-21-yne-1,42-dioate (1.50 g, 1.63 mmol) was dissolved in the mixture of CH₂Cl₂ (10 ml) and TFA (10 ml). The mixture was stirred for overnight, diluted with toluene (20 ml), concentrated to afford the title compound (1.33 g, 101% yield, ˜92% pure by HPLC) which was used for the next step without further purification. MS ESI m/z calcd for C₃₄H₅₆N₆O₁₆ [M+H]⁺ 805.37, found 805.85.

### Example 62. Synthesis of bis(2,5-dioxopyrrolidin-1-yl) 2,5,38,41-tetramethyl-4,7,20,23,36,39-hexaoxo-10,13,16,27,30,33-hexaoxa-3,6,19,24,37,40-hexaazadotetracont-21-yne-1,42-dioate

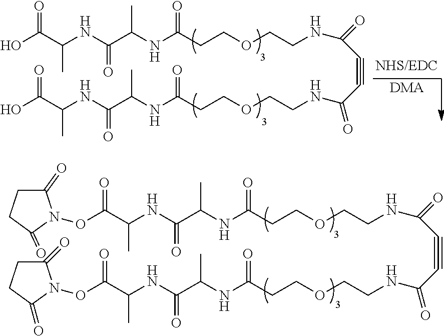

To a solution of 2,5,38,41-tetramethyl-4,7,20,23,36,39-hexaoxo-10,13,16,27,30,33-hexaoxa-3,6,19,24,37,40-hexaazadotetracont-21-yne-1,42-dioic acid (1.30 g, 1.61 mmol) in the mixture of DMA (10 ml) was added NHS (0.60 g, 5.21 mmol) and EDC (1.95 g, 10.15 mmol). The mixture was stirred for overnight, concentrated and purified on SiO₂ column eluted with EtOAc/CH₂Cl₂ (1:4 to 2:1) to afford the title compound (1.33 g, 83% yield, ˜95% pure by HPLC). MS ESI m/z calcd for C₄₂H₆₃N₈O₂₀ [M+H]⁺ 999.40, found 999.95.

### Example 63. Synthesis of 2,3-bis(2-bromoacetamido)succinyl Dichloride

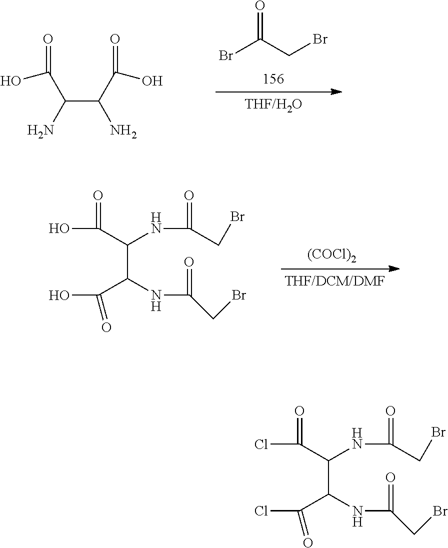 [SMILES: NC(C(=O)O)C(N)C(=O)O ; O=C(Br)CBr ; O=C(CBr)NC(C(=O)O)C(NC(=O)CBr)C(=O)O ; "(COCl)2" ; O=C(CBr)NC(C(=O)Cl)C(NC(=O)CBr)C(=O)Cl]

2,3-Diaminosuccinic acid (5.00 g, 33.77 mmol) in the mixture of THF/H₂O/DIPEA (125 ml/125 ml/8 ml) was added 2-bromoacetyl bromide (25.0 g, 125.09 mmol). The mixture was stirred for overnight, evaporated and purified by SiO₂ column chromatography (H₂O/CH₃CN 5:95) to afforded 2,3-bis(2-bromoacetamido)succinic acid (9.95 g, 76% yield) as light yellow oil. MS ESI m/z calcd for C₈H₁₁Br₂N₂O₆ [M+H]⁺ 388.89, found 388.68.

2,3-bis(2-bromoacetamido)succinic acid (3.50 g, 9.02 mmol) in dichloromethane (80 ml) was added oxalyl dichloride (5.80 g, 46.05 mmol) and DMF (0.01 ml). The mixture was stirred for 2.5 h, diluted with toluene, concentrated and co-evaporated with dichloroethane (2×20 ml) and toluene (2×15 ml) to dryness to afford 2,3-bis(2-bromoacetamido)succinyl dichloride as crude product (which is not stable) for the next step without further purification (3.90 g, 102% yield). MS ESI m/z calcd for CH₉Br₂Cl₂N₂O₄ [M+H]⁺ 424.82, found 424.90.

### Example 64. Synthesis of 2,3-bis(((benzyloxy)carbonyl)amino)succinic Acid

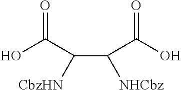 [SMILES: O=C(NC(C(=O)O)C(NC(=O)OCc1ccccc1)C(=O)O)OCc1ccccc1]

To a solution of 2,3-diaminosuccinic acid (4.05 g, 27.35 mmol) in the mixture of THF (250 ml) and NaH₂PO₄ (0.1 M, 250 ml, pH 8.0) was added benzyl carbonochloridate (15.0 g, 88.23 mmol) in 4 portions in 2 h. The mixture was stirred for another 6 h, concentrated and purified on SiO₂ column eluted with H₂O/CH₃CN (1:9) containing 1% formic acid to afford the title compound (8.65 g, 76% yield, ˜95% pure). MS ESI m/z calcd for C₂₀H₂₁N₂O₈ [M+H]⁺ 417.12, found 417.60.

### Example 65. Synthesis of bis(2,5-dioxopyrrolidin-1-yl) 2,3-bis(((benzyloxy)carbonyl)-amino)succinate

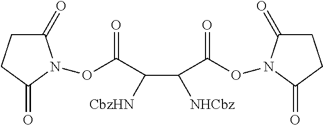 [SMILES: O=C(NC(C(=O)ON1C(=O)CCC1=O)C(NC(=O)OCc1ccccc1)C(=O)ON1C(=O)CCC1=O)OCc1ccccc1]

To a solution of 2,3-bis(((benzyloxy)carbonyl)amino)succinic acid (4.25 g, 10.21 mmol) in the mixture of DMA (70 ml) was added NHS (3.60 g, 31.30 mmol) and EDC (7.05 g, 36.72 mmol). The mixture was stirred for overnight, concentrated and purified on SiO₂ column eluted with EtOAc/CH₂Cl₂ (1:6) to afford the title compound (5.42 g, 87% yield, ˜95% pure). MS ESI m/z calcd for C₂₈H₂₇N₄O₁₂ [M+H]⁺ 611.15, found 611.60.

### Example 66. Synthesis of 2,3-bis(2,5-dioxo-2,5-dihydro-H-pyrrol-1-yl)succinic Acid

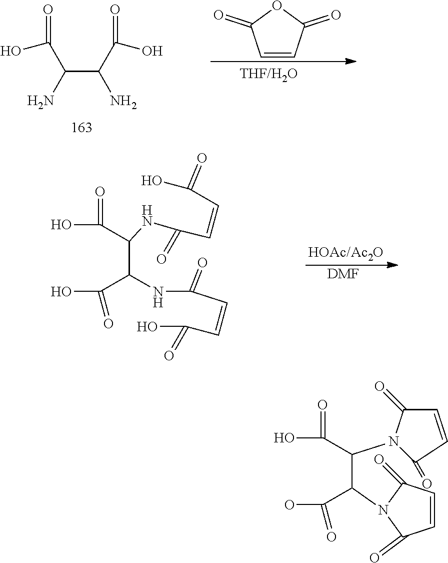

2,3-Diaminosuccinic acid (5.00 g, 33.77 mmol) in the mixture of THF/H₂O/DIPEA (125 ml/125 ml/2 ml) was added maleic anhydride (6.68 g, 68.21 mmol). The mixture was stirred for overnight, evaporated to afforded 2,3-bis((Z)-3-carboxyacrylamido)succinic acid (11.05 g, 99% yield) as a white solid. MS ESI m/z calcd for C₁₂H₁₃N₂O₁₀ [M+H]⁺ 345.05, found 345.35.

2,3-bis((Z)-3-carboxyacrylamido)succinic acid (11.05 g, 33.43 mmol) in a mixture solution of HOAc (70 ml), DMF (10 ml) and toluene (50 ml) was added acetic anhydride (30 ml). The mixture was stirred for 2 h, reflux with Dean-Stark Trap at 100° C. for 6 h, concentrated, co-evaporated with EtOH (2×40 ml) and toluene (2×40 ml), and purified on SiO₂ column eluted with H₂O/CH₃CN (1:10) to afford the title compound (7.90 g, 76% yield, ˜95% pure). MS ESI m/z calcd for C₁₂H₉N₂O₈ [M+H]⁺ 309.03, found 309.30.

### Example 67. Synthesis of Bis(2,5-dioxopyrrolidin-1-yl) 2,3-bis(2,5-dioxo-2,5-dihydro-1H-pyrrol-1-yl)succinate

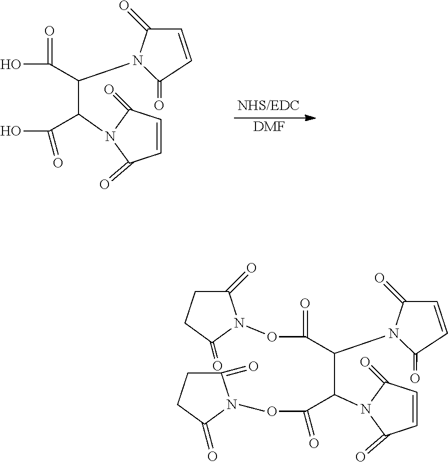

To a solution of 2,3-bis(2,5-dioxo-2,5-dihydro-1H-pyrrol-1-yl)succinic acid (4.00 g, 12.98 mmol) in the mixture of DMF (70 ml) was added NHS (3.60 g, 31.30 mmol) and EDC (7.05 g, 36.72 mmol). The mixture was stirred for overnight, concentrated and purified on SiO₂ column eluted with EtOAc/CH₂Cl₂ (1:6) to afford the title compound (5.73 g, 88% yield, ˜96% pure by HPLC). MS ESI m/z calcd for C₂₀H₁₅N₄O₁₂ [M+H]⁺ 503.06, found 503.45.

### Example 68. Synthesis of (3S,6S,39S,42S)-di-tert-butyl 6,39-bis(4-((tert-butoxycarbonyl)amino)butyl)-22,23-bis(2,5-dioxo-2,5-dihydro-1H-pyrrol-1-yl)-3,42-bis((4-(hydroxymethyl)phenyl)carbamoyl)-5,8,21,24,37,40-hexaoxo-11,14,17,28,31,34-hexaoxa-4,7,20,25,38,41-hexaazatetratetracontane-1,44-dioate

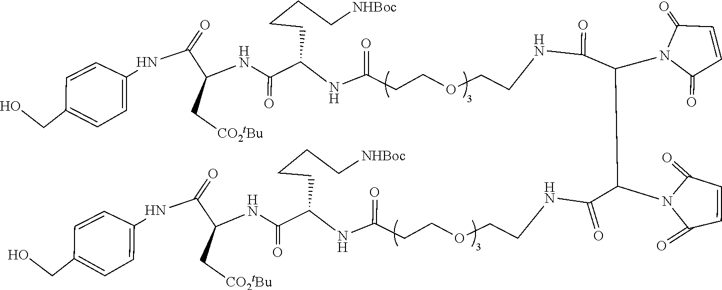

### (14S,17S)-tert-butyl 1-amino-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate

(1.43 g, 1.97 mmol) and 2,3-bis(2,5-dioxo-2,5-dihydro-1H-pyrrol-1-yl)succinic acid (0.30 g, 0.97 mmol) in DMA (25 ml) was added EDC (1.30 g, 6.77 mmol). The mixture was stirred for overnight, evaporated in vacuo, purified on silica gel using a mixture of methanol (from 5% to 8%) in methylene chloride containing as the eluant to give title compound (1.33 g, 80% yield). ESI MS m/z C₈₂H₁₂₃N₁₂O₂₈ [M+H]⁺, cacld. 1722.85, found 1722.98.

### Example 69. Synthesis of Tert-butyl 1-azido-14,17-dimethyl-12,15-dioxo-3,6,9-trioxa-13,16-diazaoctadecan-18-oate

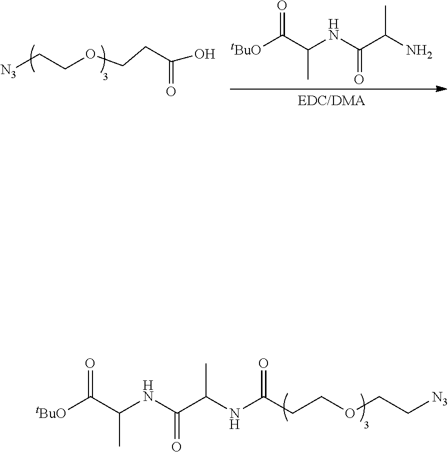

To a solution of 3-(2-(2-(2-azidoethoxy)ethoxy)ethoxy)propanoic acid (1.55 g, 6.27 mmol), tert-butyl 2-(2-aminopropanamido)propanoate (1.35 g, 6.27 mmol) in the mixture of DMA (60 ml) was added EDC (3.05 g, 15.88 mmol). The mixture was stirred for overnight, concentrated and purified on SiO₂ column eluted with EtOAc/CH₂Cl₂ (1:3) to afford the title compound (2.42 g, 86% yield, ˜95% pure by HPLC). MS ESI m/z calcd for C₁₉H₃₆N₅O₇ [M+H]+ 446.25, found 446.60.

### Example 70. Synthesis of 1-azido-14,17-dimethyl-12,15-dioxo-3,6,9-trioxa-13,16-diazaoctadecan-18-oic Acid

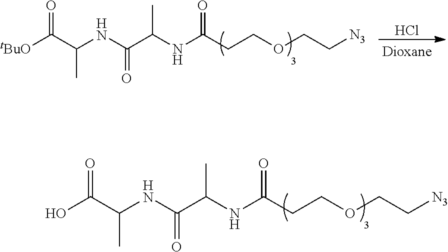

Tert-butyl 1-azido-14,17-dimethyl-12,15-dioxo-3,6,9-trioxa-13,16-diazaoctadecan-18-oate (2.20 g, 4.94 mmol) in 1,4-dioxane (40 ml) was added HCl (12 M, 10 ml). The mixture was stirred for 40 min, diluted with dioxane (20 ml) and toluene (40 ml), evaporated and co-evaporated with dioxane (20 ml) and toluene (40 ml) to dryness to afford the crude title product for the next step without further production (1.92 g, 100% yield, ˜94% pure by HPLC). MS ESI m/z calcd for C₁₅H₂₈N₅O₇ [M+H]⁺ 390.19, found 390.45.

### Example 71. Synthesis of 21,22-bis(2,5-dioxo-2,5-dihydro-H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioic Acid

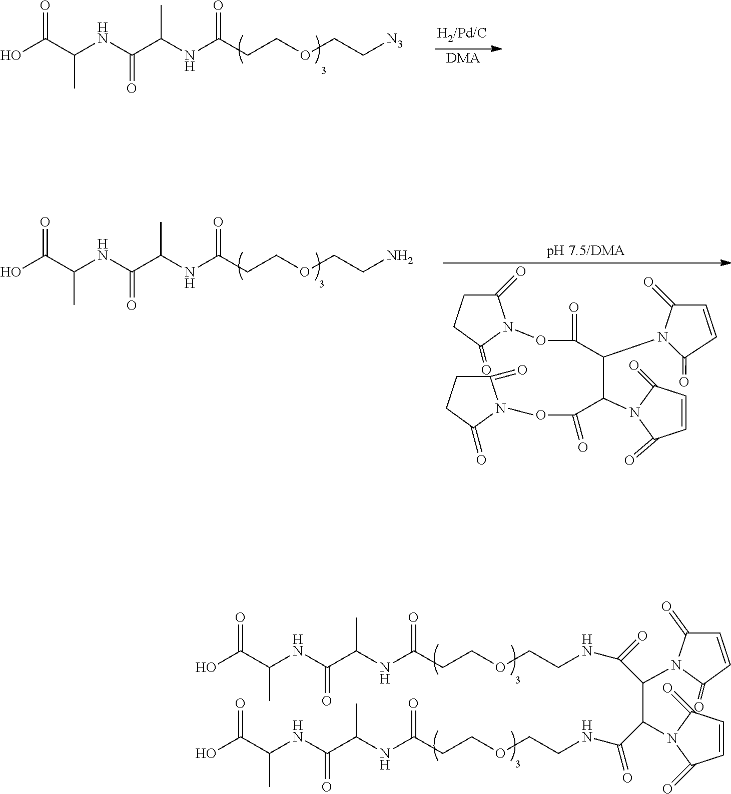

1-azido-14,17-dimethyl-12,15-dioxo-3,6,9-trioxa-13,16-diazaoctadecan-18-oic acid (1.90 g, 4.88 mmol) in DMA (40 ml) was added Pd/C (0.20 g, 50% wet). The system was evacuated under vacuum and placed under 2 atm of hydrogen gas via hydrogenation reactor with vigorous stirring. The reaction was then stirred for 6 h at room temperature and TLC showed that the starting materials disappeared. The crude reaction was passed through a short pad of Celite rinsing with ethanol. The solvent was concentrated under reduced pressure to afford the crude product, 1-amino-14,17-dimethyl-12,15-dioxo-3,6,9-trioxa-13,16-diazaoctadecan-18-oic acid in DMA which was used for the next step directly. ESI MS m/z+ C₁₅H₃₀N₃O₇ (M+H), cacld. 364.20, found 364.30.

To the amino compound in DMA (˜30 ml) was added 0.1 M NaH2PO4, pH 7.5 (20 ml), followed by addition of bis(2,5-dioxopyrrolidin-1-yl) 2,3-bis(2,5-dioxo-2,5-dihydro-1H-pyrrol-1-yl)succinate (1.30 g, 2.59 mmol). The mixture was stirred overnight, concentrated and purified on SiO₂ column eluted with 8% water on CH₃CN to afford the title compound (1.97 g, 81% yield). ESI MS m/z+ C₄₂H₆₃N₈O₂₀ (M+H), cacld. 999.41, found 999.95.

### Example 72. Synthesis of Bis(2,5-dioxopyrrolidin-1-yl) 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioate

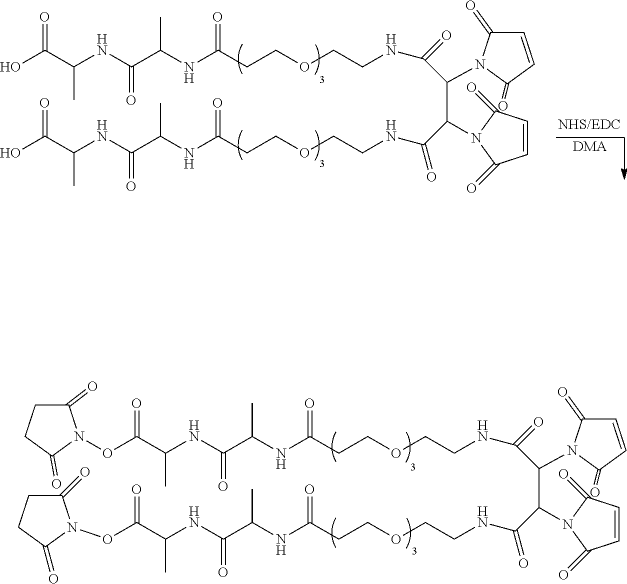

To a solution of 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioic acid (1.50 g, 1.50 mmol) in the mixture of DMA (10 ml) was added NHS (0.60 g, 5.21 mmol) and EDC (1.95 g, 10.15 mmol). The mixture was stirred for overnight, concentrated and purified on SiO₂ column eluted with EtOAc/CH₂Cl₂ (1:4 to 2:1) to afford the title compound (1.50 g, 83% yield, ˜95% pure by HPLC). MS ESI m/z calcd for C₅₀H₆₉N₁₀O₂₄ [M+H]⁺ 1193.44, found 1193.95.

### Example 73. Synthesis of (2S,4R)-methyl 4-hydroxypyrrolidine-2-carboxylate Hydrochloric

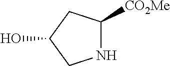

To a solution of trans-4-hydroxy-L-proline (15.0 g, 114.3 mmol) in dry methanol (250 mL) was added thionyl chloride (17 mL, 231 mmol) dropwise at 0 to 4° C. The resulting mixture was stirred for at r.t. overnight, concentrated, crystallized with EtOH/hexane to provide the title compound (18.0 g, 87% yield). ESI MS m/z 168.2 ([M+Na]⁺).

### Example 74. Synthesis of (2S,4R)-1-tert-butyl 2-methyl 4-hydroxypyrrolidine-1,2-dicarboxylate

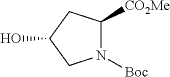 [SMILES: COC(=O)[C@@H]1C[C@@H](O)CN1C(=O)OC(C)(C)C]

To a solution of trans-4-hydroxy-L-proline methyl ester (18.0 g, 107.0 mmol) in the mixture of MeOH (150 ml) and sodium bicarbonate solution (2.0 M, 350 ml) was added Boc₂O (30.0 g, 137.6 mmol) in three portions in 4 h. After stirring for an additional 4 h, the reaction was concentrated to ˜350 ml and extracted with EtOAc (4×80 mL). The combined organic layers were washed with brine (100 mL), dried (MgSO₄), filtered, concentrated and purified by SiO₂ column chromatography (1:1 hexanes/EtOAc) to give the title compound (22.54 g, 86% yield). ESI MS m/z 268.2 ([M+Na]⁺).

### Example 75. Synthesis of (S)-1-tert-butyl 2-methyl 4-oxopyrrolidine-1,2-dicarboxylate

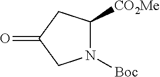 [SMILES: COC(=O)[C@@H]1CC(=O)CN1C(=O)OC(C)(C)C]

The title compound prepared through Dess-Martin oxidation was described in: Franco Manfre et al. J. Org. Chem. 1992, 57, 2060-2065. Alternatively Swern oxidation procedure is as following: To a solution of (COCl)₂ (13.0 ml, 74.38 mmol) in CH₂Cl₂ (350 ml) cooled to −78° C. was added dry DMSO (26.0 mL). The solution was stirred at −78° C. for 15 min and then (2S,4R)-1-tert-butyl 2-methyl 4-hydroxypyrrolidine-1,2-dicarboxylate (8.0 g, 32.63 mmol) in CH₂Cl₂ (100 ml) was added. After stirring at −78° C. for 2 h, triethylamine (50 ml, 180.3 mmol) was added dropwise, and the reaction solution was warmed to room temperature. The mixture was diluted with aq. NaH₂PO₄ solution (1.0 M, 400 ml) and phases separated. The aqueous layer was extracted with CH₂Cl₂ (2×60 ml). The organic layers were combined, dried over MgSO₄, filtered, concentrated and purified by SiO₂ column chromatography (7:3 hexanes/EtOAc) to give the title compound (6.73 g, 85% yield). ESI MS m/z 266.2 ([M+Na]⁺).

### Example 76. Synthesis of (S)-1-tert-butyl 2-methyl 4-methylenepyrrolidine-1,2-dicarboxylate

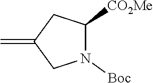 [SMILES: C=C1C[C@@H](C(=O)OC)N(C(=O)OC(C)(C)C)C1]

To a suspension of methyltriphenylphosphonium bromide (19.62 g, 55.11 mmol) in THE (150 mL) at 0° C. was added potassium-t-butoxide (6.20 g, 55.30 mmol) in anhydrous THF (80 mL). After stirring at 0° C. for 2 h, the resulting yellow ylide was added to a solution of (S)-1-tert-butyl 2-methyl 4-oxopyrrolidine-1,2-dicarboxylate (6.70 g, 27.55 mmol) in THE (40 mL). After stirring at r.t. for 1 h, the reaction mixture was concentrated, diluted with EtOAc (200 mL), washed with H₂O (150 mL), brine (150 mL), dried over MgSO₄, concentrated and purified on SiO₂ column chromatography (9:1 hexanes/EtOAc) to yield the title compound (5.77 g, 87% yield). EI MS m/z 264 ([M+Na]⁺).

### Example 77. Synthesis of (S)-methyl 4-methylenepyrrolidine-2-carboxylate Hydrochloride

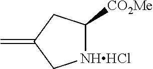

To a solution of (S)-1-tert-butyl 2-methyl 4-methylenepyrrolidine-1,2-dicarboxylate (5.70 g, 23.63 mmol) in EtOAc (40 ml) at 4° C. was added HCl (12 M, 10 ml). The mixture was stirred for 1 h, diluted with toluene (50 ml), concentrated, and crystallized with EtOH/hexane to yield the title compound as HCl salt (3.85 g, 92% yield). EI MS m/z 142.2 ([M+H]+).

### Example 78. Synthesis of (S)-tert-butyl 2-(hydroxymethyl)-4-methylenepyrrolidine-1-Carboxylate

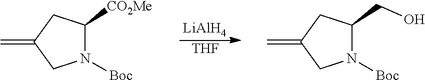 [SMILES: C=C1C[C@@H](C(=O)OC)N(C(=O)OC(C)(C)C)C1 ; "LiAlH4" ; C=C1C[C@@H](CO)N(C(=O)OC(C)(C)C)C1 ; "THF"]

To a solution of (S)-1-tert-butyl 2-methyl 4-methylenepyrrolidine-1,2-dicarboxylate. (5.20 g, 21.56 mmol) in anhydrous THE (100 mL) at 0° C. was added LiAlH₄ (15 ml, 2M in THF). After stirring at 0° C. for 4 h, the reaction was quenched by addition of methanol (5 ml) and water (20 ml). The reaction mixture was neutralized with 1 M HCl to pH 7, diluted with EtOAc (80 ml), filtered through Celite, separated and the aqueous layer was extracted with EtOAc. The organic layers were combined, dried over Na₂SO₄, concentrated and purified on SiO₂ column chromatography (1:5 EtOAc/DCM) to yield the title compound (3.77 g, 82% yield). EI MS m/z 236.40 ([M+Na]⁺).

### Example 79. Synthesis of (S)-(4-methylenepyrrolidin-2-yl)methanol, Hydrochloride Salt

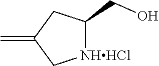

To a solution of (S)-tert-butyl 2-(hydroxymethyl)-4-methylenepyrrolidine-1-carboxylate (3.70 g, 17.36 mmol) in EtOAc (30 ml) at 4° C. was added HCl (12 M, 10 ml). The mixture was stirred for 1 h, diluted with toluene (50 ml), concentrated, and crystallized with EtOH/hexane to yield the title compound as HCl salt (2.43 g, 94% yield). EI MS m/z 115.1 ([M+H]⁺).

### Example 80. Synthesis of 4-(benzyloxy)-3-methoxybenzoic Acid

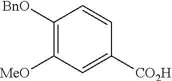 [SMILES: COc1cc(C(=O)O)ccc1OCc1ccccc1]

To a mixture of 4-hydroxy-3-methoxybenzoic acid (50.0 g, 297.5 mmol) in ethanol (350 ml) and aq. NaOH solution (2.0 M, 350 ml) was added BnBr (140.0 g, 823.5 mmol). The mixture was stirred at 65° C. for 8 h, concentrated, co-evaporated with water (2×400 ml) and concentrated to ˜400 ml, acidified to pH 3.0 with 6 N HCl. The solid was collected by filtration, crystallized with EtOH, dried at 45° C. under vacuum to afford the title compound (63.6 g, 83% yield). ESI MS m/z 281.2 ([M+Na]⁺).

### Example 81. Synthesis of 4-(benzyloxy)-5-methoxy-2-nitrobenzoic Acid

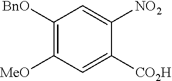 [SMILES: COc1cc(C(=O)O)c([N+](=O)[O-])cc1OCc1ccccc1]

To a solution of 4-(benzyloxy)-3-methoxybenzoic acid (63.5 g, 246.0 mmol) in CH₂Cl₂ (400 ml) and HOAc (100 ml) was added HNO₃ (fuming, 25.0 ml, 528.5 mmol). The mixture was stirred for 6 h, concentrated, crystallized with EtOH, dried at 40° C. under vacuum to afford the title compound (63.3 g, 85% yield). ESI MS m/z 326.1 ([M+Na]⁺).

### Example 82. Synthesis of (S)-(4-(benzyloxy)-5-methoxy-2-nitrophenyl)(2-(hydroxymethyl)-4-methylenepyrrolidin-1-yl)methanone

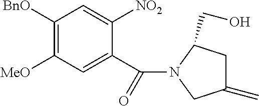 [SMILES: C=C1C[C@@H](CO)N(C(=O)c2cc(OC)c(OCc3ccccc3)cc2[N+](=O)[O-])C1]

A catalytic amount of DMF (30 μl) was added to a solution of 4-(benzyloxy)-5-methoxy-2-nitrobenzoic acid (2.70 g, 8.91 mmol) and oxalyl chloride (2.0 mL, 22.50 mmol) in anhydrous CH₂Cl₂ (70 mL) and the resulting mixture was stirred at room temperature for 2 h. Excess CH₂Cl₂ and oxalyl chloride was removed with rotavap. The acetyl chloride was re-suspended in fresh CH₂Cl₂ (70 mL) and was added slowly to a pre-mixed solution of (S)-(4-methylenepyrrolidin-2-yl)methanol, hydrochloride salt (1.32 g, 8.91 mmol) and Et₃N (6 mL) in CH₂Cl₂ at 0° C. under N2 atmosphere. The reaction mixture was allowed to warm to r.t. and stirring was continued for 8 h. After removal of CH₂Cl₂ and Et₃N, the residue was partitioned between H₂O and EtOAc (70/70 mL). The aqueous layer was further extracted with EtOAc (2×60 mL). The combined organic layers were washed with brine (40 mL), dried (MgSO₄) and concentrated. Purification of the residue with flash chromatography (silica gel, 2:8 hexanes/EtOAc) yielded the title compound (2.80 g, 79% yield). EI MS m/z 421.2 ([M+Na]+).

### Example 83. Synthesis of (S)-(4-(benzyloxy)-5-methoxy-2-nitrophenyl)(2-(((tert-butyldimethylsilyl)oxy)methyl)-4-methylenepyrrolidin-1-yl)methanone

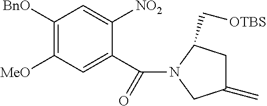 [SMILES: C=C1C[C@@H](CO[Si](C)(C)C(C)(C)C)N(C(=O)c2cc(OC)c(OCc3ccccc3)cc2[N+](=O)[O-])C1]

(S)-(4-(Benzyloxy)-5-methoxy-2-nitrophenyl)(2-(hydroxymethyl)-4-methylenepyrrolidin-1-yl)methanone (2.78 g, 8.52 mmol) in the mixture of DCM (10 ml) and pyridine (10 ml) was added tert-butylchlorodimethylsilane (2.50 g, 16.66 mmol). The mixture was stirred for overnight, concentrated and purified on SiO₂ column eluted with EtOAc/CH₂Cl₂ (1:6) to afford the title compound (3.62 g, 83% yield, ˜95% pure). MS ESI m/z calcd for C₂₇H₃₇N₂O₆Si [M+H]⁺ 513.23, found 513.65.

### Example 84. Synthesis of (S)-(4-hydroxy-5-methoxy-2-nitrophenyl)(2-(hydroxymethyl)-4-methylenepyrrolidin-1-yl)methanone

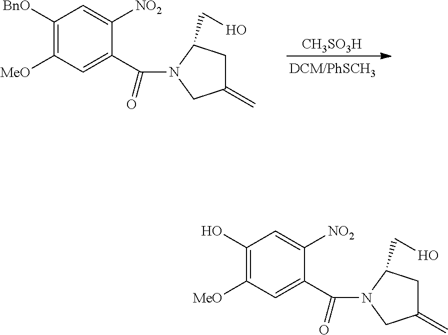

(S)-(4-(Benzyloxy)-5-methoxy-2-nitrophenyl)(2-(hydroxymethyl)-4-methylenepyrrolidin-1-yl)methanone (2.80 g, 7.03 mmol) in the mixture of DCM (30 ml) and CH₃SO₃H (8 ml) was added PhSCH₃ (2.00 g, 14.06 mmol). The mixture was stirred for 0.5 h, diluted with DCM (40 ml), neutralized with carefully addition of 0.1 M Na₂CO₃ solution. The mixture was separated and the aqueous solution was extracted with DCM (2×10 ml). The organic layers were combined, dried over Na₂SO₄, concentrated and purified on SiO₂ column eluted with MeOH/CH₂Cl₂ (1:15 to 1:6) to afford the title compound (1.84 g, 85% yield, ˜95% pure). MS ESI m/z calcd for C₁₄H₁₇N₂O₆ [M+H]⁺ 309.10, found 309.30.

### Example 85. Synthesis of (S)-((pentane-1,5-diylbis(oxy))bis(5-methoxy-2-nitro-4,1-phenylene))bis(((S)-2-(hydroxymethyl)-4-methylenepyrrolidin-1-yl)methanone)

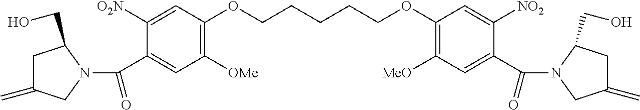

(S)-(4-hydroxy-5-methoxy-2-nitrophenyl)(2-(hydroxymethyl)-4-methylenepyrrolidin-1-yl)methanone (0.801 g, 2.60 mmol) in butanone (10 ml) was added Cs₂CO₃, (2.50 g, 7.67 mmol), followed by addition of 1,5-diiodopentane (415 mmol, 1.28 mmol). The mixture was stirred for 26 h, concentrated and purified on SiO₂ column eluted with MeOH/CH₂Cl₂ (1:15 to 1:5) to afford the title compound (0.675 g, 77% yield, ˜95% pure). MS ESI m/z calcd for C₃₃H₄₁N₄O₁₂ [M+H]+ 685.26, found 685.60.

### Example 86. Synthesis of (S)-((pentane-1,5-diylbis(oxy))bis(2-amino-5-methoxy-4,1-phenylene))bis(((S)-2-(hydroxymethyl)-4-methylenepyrrolidin-1-yl)methanone)

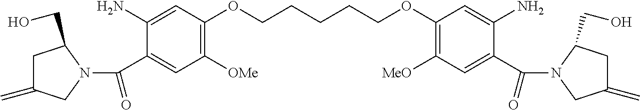

(S)-((pentane-1,5-diylbis(oxy))bis(5-methoxy-2-nitro-4,1-phenylene))bis(((S)-2-(hydroxymethyl)-4-methylenepyrrolidin-1-yl)methanone) (0.670 g, 0.98 mmol) in CH₃OH (10 ml) was added Na₂S₂O₄ (1.01 g, 5.80 mmol) in H₂O (8 ml). The mixture was stirred at room temperature for 30 h. The reaction mixture was evaporated and co-evaporated with DMA (2×10 mL) and EtOH (2×10 ml)under high vacuum to dryness to afford the title compound (total weight 1.63 g) containing inorganic salts which was used directly for the next step reaction (without further separation). EIMS m/z 647.32 ([M+Na]⁺).

### Example 87. Synthesis of C-1 (a PBD Dimer Analog Having a Bis-Linker)

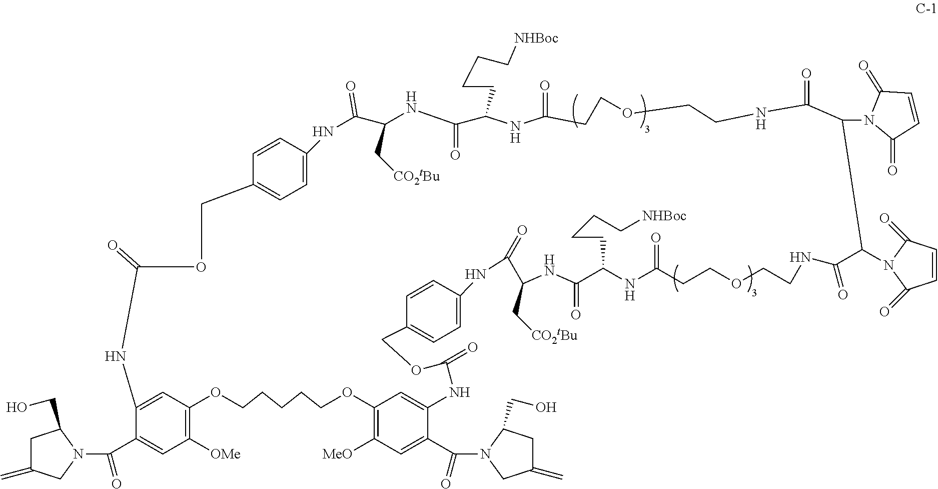

(3 S,6S,39S,42S)-di-tert-butyl 6,39-bis(4-((tert-butoxycarbonyl)amino)butyl)-22,23-bis(2,5-dioxo-2,5-dihydro-1H-pyrrol-1-yl)-3,42-bis((4-(hydroxymethyl)phenyl)carbamoyl)-5,8,21,24,37,40-hexaoxo-11,14,17,28,31,34-hexaoxa-4,7,20,25,38,41-hexaazatetratetracontane-1,44-dioate (0.840 g, 0.488 mmol) in THE (8 mL) containing pyridine (0.100 ml, 1.24 mmol) at 0° C. was added dropwise of a solution of triphosgene (0.290 mg, 0.977 mmol) in THE (3.0 mL). The reaction mixture was stirred at 0° C. for 15 min then was used directly in the next step.

(S)-((pentane-1,5-diylbis(oxy))bis(2-amino-5-methoxy-4,1-phenylene))bis(((S)-2-(hydroxymethyl)-4-methylenepyrrolidin-1-yl)methanone) containing inorganic salts (0.842 mg, ˜0.49 mmol) was suspended in EtOH (10 ml) at 0° C. was added the trichloride in THE prepared above. The mixture was stirred at 0° C. for 4 h, then warmed to RT for 1 h, concentrated, and purified by reverse phase HPLC (250 (L) mm x 10(d) mm, C₁₈ column, 10-80% acetonitrile/water in 40 min, v=8 ml/min) to afford the title compound (561.1 mg, 48% yield in three steps). ESI MS m/z: calcd for C₁₁₇H₁₆₃N₁₆O₃₈ [M+H]⁺ 2400.12, found 2400.90.

### Example 88. Synthesis of C-2 (a PBD Dimer Analog Having a Bis-Linker)

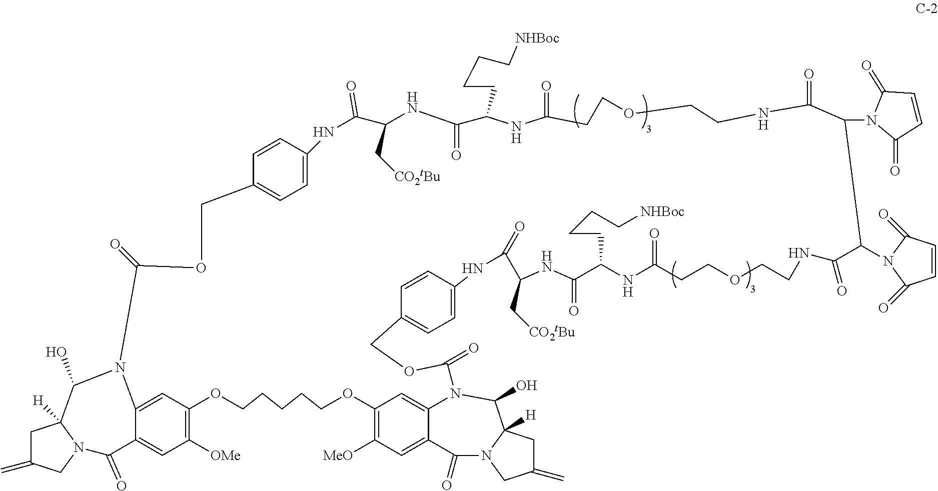

Dess-Martin periodinane (138.0 mg, 0.329 mmol) was added to a solution of compound C-1 (132.0 mg, 0.055 mmol) in DCM (5.0 mL) at 0° C. The reaction mixture was warmed to RT and was stirred for 2 h. A saturated solution of NaHCO₃/Na₂SO₃ (5.0 mL/5.0 mL) was then added and the mixture was extracted with DCM (3×25 mL). The combined organic layers were washed with NaHCO₃/Na₂SO₃ (5.0 mL/5.0 mL), brine (10 mL), dried over Na₂SO₄, filtered, concentrated and purified by reverse phase HPLC (250 (L) mm x 10(d) mm, C₁₈ column, 10-80% acetonitrile/water in 40 min, v=8 ml/min) to afford the title compound (103.1 mg, 78% yield) as a foam ESI MS m/z: calcd for C₁₁₇H₁₅₈N₁₆O₃₈ [M+H]⁺ 2396.09, found 2396.65.

### Example 89. Synthesis of C-3 (a PBD Dimer Analog Having a Bis-Linker)

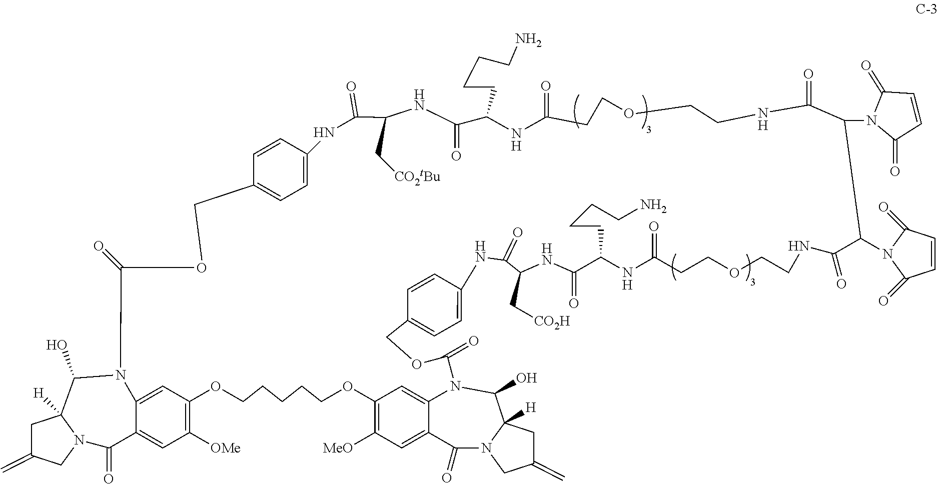

C-2 compound (55.0 mg, 0.023 mmol) was dissolved in DCM (3 ml), followed by addition of TFA (3 ml) at 4° C. The reaction mixture was then stirred at RT for 1 h, then concentrated, and co-evaporated with DCM/toluene to dryness to afford the crude product C-3 (48.0 mg, 100% yield, 92% pure by HPLC) which was further purified by reverse phase HPLC (250 (L) mm x 20(d) mm, C₁₈ column, 5-60% acetonitrile/water in 40 min, v=8 ml/min) to afford the pure product C-3 (42.1 mg, 88% yield, 96% pure) as a foam. ESI MS m/z: calcd for C₉₉H₁₂₆N₁₆O₃₄ [M+H]⁺ 2083.86, found 2084.35.

### Example 90. Synthesis of C-4 (a PBD Dimer Analog Having a Bis-Linker)

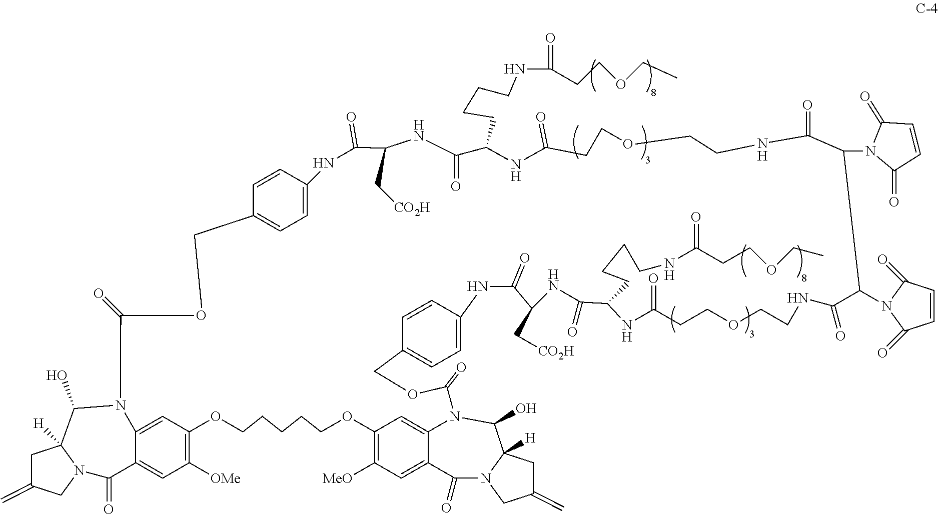

C-3 compound (35.0 mg, 0.017 mmol) was dissolved in a mixture solution of THF (3 ml) and 0.1 M, NaH₂PO₄ (3 ml), pH 7.5, followed by addition of N-succinimidyl 2,5,8,11,14,17,20,23-octaoxahexacosan-26-oate (43.0 mg, 0.084 mmol) in 4 portions in 2 h. The reaction mixture was then continued to stir at RT for 4 h, and co-evaporated with DMF (10 ml) to dryness to afford the crude product C-4 which was further purified by reverse phase HPLC (250 (L) mm x 20(d) mm, C₁₈ column, 20-60% acetonitrile/water in 40 min, v=8 ml/min) to afford the pure product C-4 (39.4 mg, 81% yield, 96% pure) as a foam. ESI MS m/z: calcd for C₁₃₅H₁₉₅N₁₆O₅₂ [M+H]⁺ 2872.30, found 2871.65.

### Example 91. Synthesis of C-5 (a PBD Dimer Analog Having a Bis-Linker)

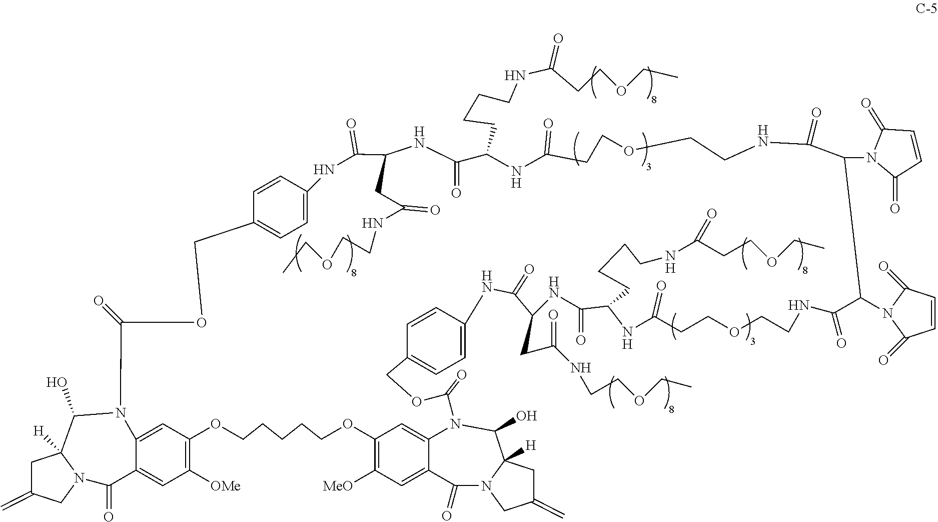

To a solution of C-4 compound (35.0 mg, 0.012 mmol) and 2,5,8,11,14,17,20,23-octaoxapentacosan-25-amine (15.1 mg, 0.0394 mmol) in dry DMA (2 ml) was added EDC (30.0 mg, 0.156 mmol). The reaction mixture was stirred at RT for 14 h, concentrated, purified by reverse phase HPLC (250 (L) mm x 20(d) mm, C₁₈ column, 20-60% acetonitrile/water in 40 min, v=8 ml/min) to afford the pure product C-5 (31.2 mg, 77% yield, 97% pure by HPLC) as a foam. ESI MS m/z: calcd for C₁₆₁H₂₄₉N₁₈O₆₂ [M+H]⁺ 3426.68, found 3427.21.

### Example 92. Synthesis of (S)-methyl 1-(4-(benzyloxy)-5-methoxy-2-nitrobenzoyl)-4-methylenepyrrolidine-2-carboxylate

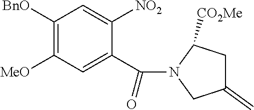 [SMILES: C=C1C[C@@H](C(=O)OC)N(C(=O)c2cc(OC)c(OCc3ccccc3)cc2[N+](=O)[O-])C1]

A catalytic amount of DMF (30 μl) was added to a solution of 4-(benzyloxy)-5-methoxy-2-nitrobenzoic acid (2.70 g, 8.91 mmol) and oxalyl chloride (2.0 mL, 22.50 mmol) in anhydrous CH₂Cl₂ (70 mL) and the resulting mixture was stirred at room temperature for 2 h. Excess CH₂Cl₂ and oxalyl chloride was removed with rotavap. The acetyl chloride was re-suspended in fresh CH₂Cl₂ (70 mL) and was added slowly to a pre-mixed solution of (S)-methyl 4-methylenepyrrolidine-2-carboxylate hydrochloride (1.58 g, 8.91 mmol) and Et₃N (6 mL) in CH₂Cl₂ at 0° C. under N2 atmosphere. The reaction mixture was allowed to warm to r.t. and stirring was continued for 8 h. After removal of CH₂Cl₂ and Et₃N, the residue was partitioned between H₂O and EtOAc (70/70 mL). The aqueous layer was further extracted with EtOAc (2×60 mL). The combined organic layers were washed with brine (40 mL), dried (MgSO₄) and concentrated. Purification of the residue with flash chromatography (silica gel, 2:8 hexanes/EtOAc) yielded the title compound (2.88 g, 76% yield). EI MS m/z 449.1 ([M+Na]⁺).

### Example 93. Synthesis of (S)-1-(4-(benzyloxy)-5-methoxy-2-nitrobenzoyl)-4-methylenepyrro-lidine-2-carbaldehyde

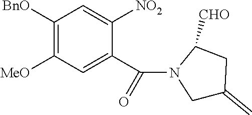 [SMILES: C=C1C[C@@H](C=O)N(C(=O)c2cc(OC)c(OCc3ccccc3)cc2[N+](=O)[O-])C1]

To a vigorously stirred solution of (S)-methyl 1-(4-(benzyloxy)-5-methoxy-2-nitro benzoyl)-4-methylenepyrrolidine-2-carboxylate (2.80 g, 6.57 mmol) in anhydrous CH₂Cl₂ (60 mL) was added DIBAL-H (1N in CH₂Cl₂, 10 mL) dropwise at −78° C. under N2 atmosphere. After the mixture was stirred for an additional 90 min, excess reagent was decomposed by addition of 2 ml of methanol, followed by 5% HCl (10 mL). The resulting mixture was allowed to warm to 0° C. Layers were separated and the aqueous layer was further extracted with CH₂Cl₂ (3×50 mL). Combined organic layers were washed with brine, dried (MgSO₄) and concentrated. Purification of the residue with flash chromatography (silica gel, 95:5 CHCl₃/MeOH) yielded the title compound (2.19 g, 84% yield). EIMS m/z 419.1 ([M+Na]⁺).

### Example 94. Synthesis of (S)-8-(benzyloxy)-7-methoxy-2-methylene-2,3-dihydro-1H-benzo[e]-pyrrolo[1,2-a]azepin-5(11aH)-one

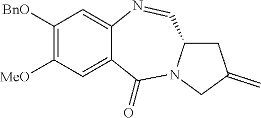 [SMILES: C=C1C[C@H]2C=Nc3cc(OCc4ccccc4)c(OC)cc3C(=O)N2C1]

A mixture of (S)-1-(4-(benzyloxy)-5-methoxy-2-nitrobenzoyl)-4-methylenepyrro-lidine-2-carbaldehyde (2.18 g, 5.50 mmol) and Na₂S₂O₄ (8.0 g, 45.97 mmol) in THE (60 ml) and H₂O (40 ml) was stirred at room temperature for 20 h. Solvents were removed under high vacuum. The residue was re-suspended in MeOH (60 mL), and HCl (6M) was added dropwise until pH 2 was reached. The resulting mixture was stirred at r.t. for 1 h. The reaction was worked-up by removing most of MeOH, then diluted with EtOAc (100 mL). The EtOAc solution was washed with sat. NaHCO₃, brine, dried (MgSO₄), and concentrated. Purification of the residue with flash chromatography (silica gel, 97:3 CHCl₃/MeOH) yielded the title compound (1.52 g, 80%). EIMS m/z 372.1 ([M+Na]⁺).

### Example 95. Synthesis of (S)-8-hydroxy-7-methoxy-2-methylene-2,3-dihydro-1H-benzo[e]-pyrrolo[1,2-a]azepin-5(11aH)-one

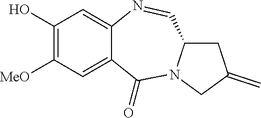 [SMILES: C=C1C[C@H]2C=Nc3cc(O)c(OC)cc3C(=O)N2C1]

To a solution of (S)-8-(benzyloxy)-7-methoxy-2-methylene-2,3-dihydro-1H-benzo[e]-pyrrolo[1,2-a]azepin-5(11aH)-one (1.50 g, 4.32 mmol) in 70 ml of CH₂Cl₂ was added 25 ml of CH₃SO₃H at 0° C. The mixture was stirred at 0° C. for 10 min then r.t. for 2 h, diluted with CH₂Cl₂, pH adjusted with cold 1.0 N NaHCO₃ to 4 and filtered. The aqueous layer was extracted with CH₂Cl₂ (3×60 ml). The organic layers were combined, dried over Na₂SO₄, filtered, evaporated and purified on SiO₂ column chromatography (CH₃OH/CH₂Cl₂ 1:15) to afford 811 mg (73% yield) of the title product. EIMS m/z 281.1 ([M+Na]⁺).

### Example 96. Synthesis of (11aS,11a′S)-8,8′-(pentane-1,5-diylbis(oxy))bis(7-methoxy-2-methylene-2,3-dihydro-1H-benzo[e]pyrrolo[1,2-a][1,4]diazepin-5(11aH)-one)

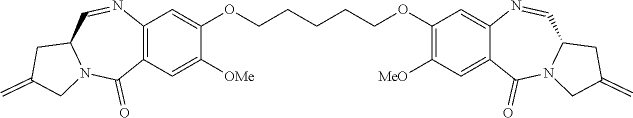

To a stirred suspended solution of Cs₂CO₃ (0.761 g, 2.33 mmol) in butanone (8 ml) were added (S)-8-hydroxy-7-methoxy-2-methylene-2,3-dihydro-1H-benzo[e]-pyrrolo[1,2-a]azepin-5(11aH)-one (401 mg, 1.55 mmol) and 1,5-diiodopentane (240 mg, 0.740 mmol). The mixture was stirred at r.t. overnight, concentrated, and purified on SiO₂ chromatography (EtOAc/CH₂Cl₂ 1:10) to afford 337 mg (78% yield) of the title product. EIMS m/z 607.2 ([M+Na]⁺).

### Example 97. Synthesis of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one and (11aS,11a′S)-8,8′-(pentane-1,5-diylbis(oxy))bis(7-methoxy-2-methylene-2,3,11,11a-tetrahydro-1H-benzo[e]pyrrolo[1,2-a][1,4]diazepin-5(10H)-one)

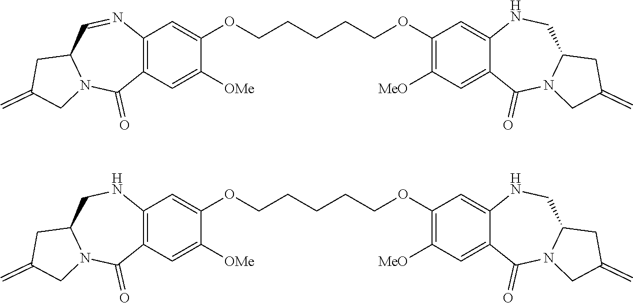

To a solution of (11aS,11a′S)-8,8′-(pentane-1,5-diylbis(oxy))bis(7-methoxy-2-methylene-2,3-dihydro-1H-benzo[e]pyrrolo[1,2-a][1,4]diazepin-5(11aH)-one) (150 mg, 0.256 mmol) in anhydrous dichloromethane (1 mL) and absolute ethanol (1.5 mL) was added sodium borohydride in methoxyethyl ether (85p, 0.5 M, 0.042 mmol) at 0° C. The ice bath was removed after 5 minutes and the mixture was stirred at room temperature for 3 hours, then cooled to 0° C., quenched with saturated ammonium chloride, diluted with dichloromethane, and phases separated. The organic layer was washed with brine, dried over anhydrous Na₂SO₄, filtered through Celite and concentrated. The residue was purified by reverse phase HPLC (C₁₈ column, acetonitrile/water). The corresponding fractions were extracted with dichloromethane and concentrated to afford the half reduced compound, (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one (64.7 mg, 43%), MS m/z 609.2 ([M+Na]⁺), 625.3 ([M+K]⁺) and 627.2 ([M+Na+H₂O]⁺); the fully reduced compound, (11aS,11a′S)-8,8′-(pentane-1,5-diylbis(oxy))bis(7-methoxy-2-methylene-2,3,11,11a-tetrahydro-1H-benzo[e]pyrrolo[1,2-a][1,4]diazepin-5(10H)-one) (16.5 mg, 11%), MS m/z 611.2 ([M+Na]⁺), 627.2 ([M+K]⁺), 629.2 ([M+Na+H₂O]⁺); and the unreacted starting material was also recovered (10.2 mg, 7%), MS m/z 607.2 ([M+Na]⁺), 625.2 ([M+Na+H₂O]⁺).

### Example 98. Synthesis of (S)-8-((5-(((S)-10-(3-(2-(2-azidoethoxy)ethoxy) propanoyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-2,3-dihydro-1H-benzo[e]pyrrolo[1,2-a][1,4]diazepin-5(11aH)-one

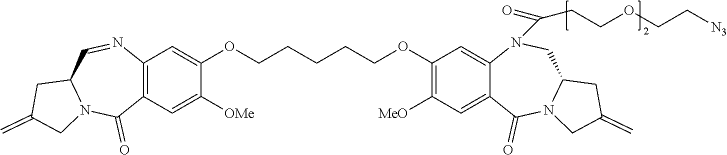

To the mixture of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one (60.0 mg, 0.102 mmol) and 2,5-dioxopyrrolidin-1-yl 3-(2-(2-azidoethoxy)ethoxy)propanoate (40.5 mg, 0.134 mmol) in dichloromethane (5 ml) was added EDC (100.5 mg, 0.520 mmol). The mixture was stirred at r.t. overnight, concentrated and purified on SiO₂ column chromatography (EtOAc/CH₂Cl₂, 1:6) to afford 63.1 mg (81% yield) of the title product. ESI MS m/z C₄₀H₅₀N₇O₉ [M+H]+, cacld. 772.36, found 772.30.

### Example 99. Synthesis of (S)-8-((5-(((S)-10-(3-(2-(2-aminoethoxy)ethoxy) Propanoyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-2,3-dihydro-1H-benzo[e]pyrrolo[1,2-a][1,4]diazepin-5(11aH)-one

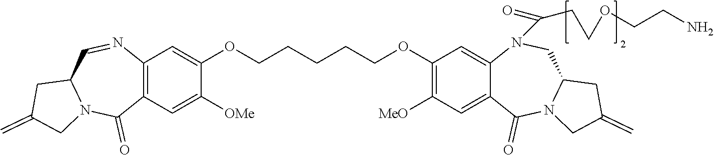

To a solution of (S)-8-((5-(((S)-10-(3-(2-(2-azidoethoxy)ethoxy) propanoyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-2,3-dihydro-1H-benzo[e]pyrrolo[1,2-a][1,4]diazepin-5(11aH)-one (60 mg, 0.078 mmol) in the mixture of THF (5 ml) and NaH₂PO₄ buffer solution (pH 7.5, 1.0 M, 0.7 ml) was added PPh₃ (70 mg, 0.267 mmol). The mixture was stirred at r.t. overnight, concentrated and purified on C₁₈ preparative HPLC, eluted with water/CH₃CN (from 90% water to 35% water in 35 min) to afford 45.1 mg (79% yield) of the title product after drying under high vacuum. ESI MS m/z C₄₀H₅₂NO₉ [M+H]⁺, cacld. 746.37, found 746.50.

### Example 100. Synthesis of(S)—N-(2-((S)-8-((5-(((11S,11aS)-10-((S)-15-azido-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)-oxy)-7-methoxy-2-methylene-5-oxo-2,3,11,11a-tetrahydro-1H-benzo[e]pyrrolo[1,2-a][1,4]diazepin-10(5H)-yl)-2-oxoethyl)-2-(3-(2-(2-azidoethoxy)ethoxy)propanamido)-3-methylbutanamide

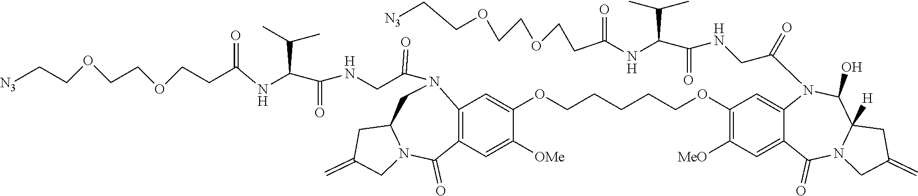 [SMILES: C=C1C[C@H]2CN(C(=O)CNC(=O)[C@@H](NC(=O)CCOCCOCCN=[N+]=[N-])C(C)C)c3cc(OCCCCCOc4cc5c(cc4OC)C(=O)N4CC(=C)C[C@H]4[C@H](O)N5C(=O)CNC(=O)[C@@H](NC(=O)CCOCCOCCN=[N+]=[N-])C(C)C)c(OC)cc3C(=O)N2C1]

To the mixture of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one (60.0 mg, 0.102 mmol) and (S)-15-azido-5-isopropyl-4,7-dioxo-10,13-dioxa-3,6-diazapentadecan-1-oic acid (90.2 mg, 0.25 mmol) in DMA (8 ml) was added BrOP (240.2 mg, 0.618 mmol). The mixture was stirred at 60° C. overnight, concentrated and purified on SiO₂ column chromatography (CH₃OH/CH₂Cl₂, 1:10 to 1:5) to afford 97.1 mg (74% yield) of the title product. ESI MS m/z C₆₁H₈₇N₁₄O₁₇ [M+H]⁺, cacld. 1287.63, found 1287.95.

### Example 101. Synthesis of (S)—N-(2-((S)-8-((5-(((11S,11aS)-10-((S)-15-amino-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,11,11a-tetrahydro-1H-benzo[e]-pyrrolo[1,2-a][1,4]diazepin-10(5H)-yl)-2-oxoethyl)-2-(3-(2-(2-aminoethoxy)ethoxy)-propanamido)-3-methylbutanamide (C-6)

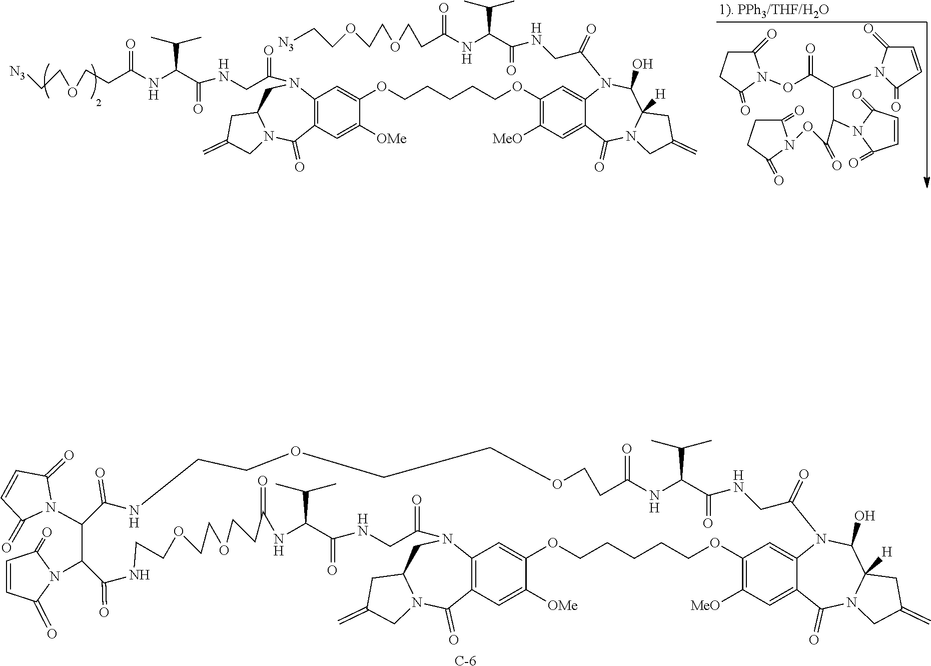

To a solution of (S)—N-(2-((S)-8-((5-(((11S,11aS)-10-((S)-15-azido-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)-oxy)-7-methoxy-2-methylene-5-oxo-2,3,11,11a-tetrahydro-1H-benzo[e]pyrrolo[1,2-a][1,4]diazepin-10(5H)-yl)-2-oxoethyl)-2-(3-(2-(2-azidoethoxy)ethoxy)propanamido)-3-methylbutanamide (85 mg, 0.066 mmol) in the mixture of THF (5 ml) was added PPh₃ (100 mg, 0.381 mmol). The mixture was stirred for 2 h, NaH₂PO₄ buffer solution (pH 7.5, 1.0 M, 0.7 ml) was then added and the mixture was stirred for 10 min. After confirmed by LC-MS to form (S)—N-(2-((S)-8-((5-(((11S,11aS)-10-((S)-15-amino-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,11,11a-tetrahydro-1H-benzo[e]pyrrolo[1,2-a][1,4]diazepin-10(5H)-yl)-2-oxoethyl)-2-(3-(2-(2-aminoethoxy)ethoxy)-propanamido)-3-methylbutanamide (ESI MS m/z C₆₁H₉₀N₁₀O₁₇ [M+Na]⁺, cacld. 1257.66, found 1257.90), bis(2,5-dioxopyrrolidin-1-yl) 2,3-bis(2,5-dioxo-2,5-dihydro-1H-pyrrol-1-yl)succinate (33 mg, 0.066 mmol) was added. The mixture was continued to stir for 4 h, concentrated and purified on C₁₈ preparative HPLC, eluted with water/CH₃CN (from 90% water to 30% water in 35 min) to afford 40.1 mg (40% yield) of the title product C-5 after drying under high vacuum. ESI MS m/z C₇₃H₉₅N₂O₂₃ [M+H]⁺, cacld. 1507.66, found 1507.90.

### Example 102. Synthesis of 4,4′-(pentane-1,5-diylbis(oxy))bis(3-methoxybenzoic Acid)

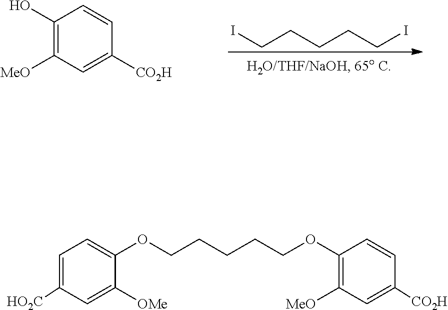 [SMILES: COc1cc(C(=O)O)ccc1O ; ICCCCCI ; COc1cc(C(=O)O)ccc1OCCCCCOc1ccc(C(=O)O)cc1OC]

A solution of diiodopropane (19.0 g, 58.6 mmol) in THE (75 mL) was added dropwise over a period of 4 hours to a vigorously stirred solution of vanilic acid (20.0 g, 119 mmol) in THE (150 mL) and aqueous NaOH (340 mL) at 65° C. in the absence of light (foil-wrapped flask). After heating at reflux for 48 hours in the dark, the solution was cooled and the THF removed by evaporation in vacuo. The residue was extracted with EA, The aqueous layer was separated and acidified to pH 2 with conc. HCl. The resultant precipitate collected by filtration, washed, dried and recrystallised from glacial acetic acid to afford the corresponding bis-carboxylic acid (14.0 g, 34.7 mmol). White solid, yield (60%).

### Example 103. Synthesis of 4,4′-(pentane-1,5-diylbis(oxy))bis(5-methoxy-2-nitrobenzoic Acid)

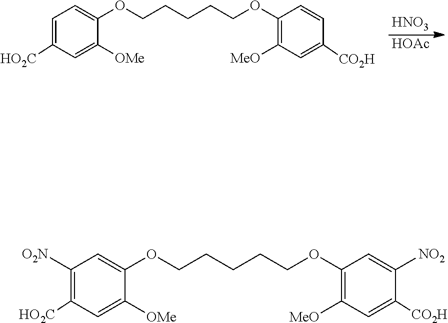 [SMILES: COc1cc(C(=O)O)ccc1OCCCCCOc1ccc(C(=O)O)cc1OC ; "HNO3" ; COc1cc(C(=O)O)c([N+](=O)[O-])cc1OCCCCCOc1cc([N+](=O)[O-])c(C(=O)O)cc1OC ; "HOAc"]

To a suspension of 4,4′-(pentane-1,5-diylbis(oxy))bis(3-methoxybenzoic acid) (18.0 g, 66.8 mmol) in HOAc (80 mL, 1800 mmol) was added HNO₃ (80 mL, 1778 mmol) dropwise at room temperature. After 2 h of stirring, the mixture was poured into 100 g ice and extracted with EA (2×200 mL). The organic layer was separated and washed with H₂O (2×100 mL), then 4N NaOH (400 mL) was added. After extracted with EA (2×100 mL), the basic aqueous layer was separated and acidified to pH 2 with conc. HCl. The mixture was extracted with EA (2×250 mL). The combined organic extract was washed with brine, dried, filtered and concentrated. The residue was purified by flash chromatography (DCM/MeOH=4/1) to give 4,4′-(pentane-1,5-diylbis(oxy))bis(5-methoxy-2-nitrobenzoic acid) (6.1 g, 12.3 mmol) as a pale yellow solid in 18% yield. R_{f} 0.3 (DCM/MeOH=3/1)

### Example 104. Synthesis of (S)-((pentane-1,5-diylbis(oxy))bis(5-methoxy-2-nitro-4,1-phenylene))bis(((S)-2-(hydroxymethyl)pyrrolidin-1-yl)methanone)

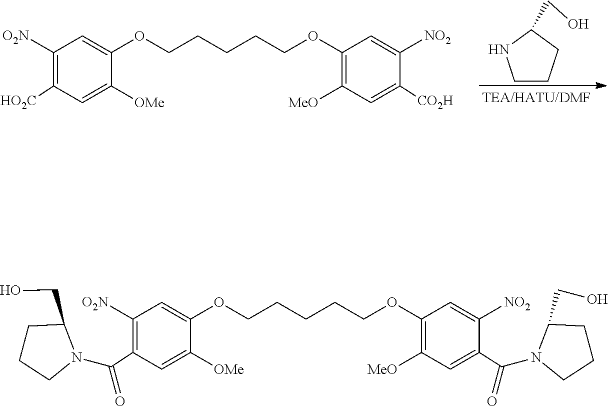 [SMILES: COc1cc(C(=O)O)c([N+](=O)[O-])cc1OCCCCCOc1cc([N+](=O)[O-])c(C(=O)O)cc1OC ; OC[C@@H]1CCCN1 ; COc1cc(C(=O)N2CCC[C@H]2CO)c([N+](=O)[O-])cc1OCCCCCOc1cc([N+](=O)[O-])c(C(=O)N2CCC[C@H]2CO)cc1OC]

To a solution of 4,4′-(pentane-1,5-diylbis(oxy))bis(5-methoxy-2-nitrobenzoic acid) (5.0 g, 10.0 mmol) and L-(+)-Prolinol (2.25 g, 22.3 mmol) in DMF (100 mL) was added TEA (4.0 g) at room temperature. After 10 min of stirring, HATU (10.77 g, 28.3 mmol) was added. The mixture was stirred at room temperature overnight. After completion of conversion, the mixture was diluted with H₂O (100 mL) and extracted with EA (2×100 mL) and DCM (2×50 mL), the combined organic extract was washed with brine, dried, filtered and concentrated. The residue was purified by chromatography (DCM/MeOH=15/1) to give (S)-((pentane-1,5-diylbis(oxy))bis(5-methoxy-2-nitro-4,1-phenylene))bis(((S)-2-(hydroxymethyl)pyrrolidin-1-yl)methanone) (6.0 g, 9.1 mmol) as a white foam in 91% yield.

### Example 105. Synthesis of (S)-((pentane-1,5-diylbis(oxy))bis(2-amino-5-methoxy-4,1-phenylene))bis(((S)-2-(hydroxymethyl)pyrrolidin-1-yl)methanone)

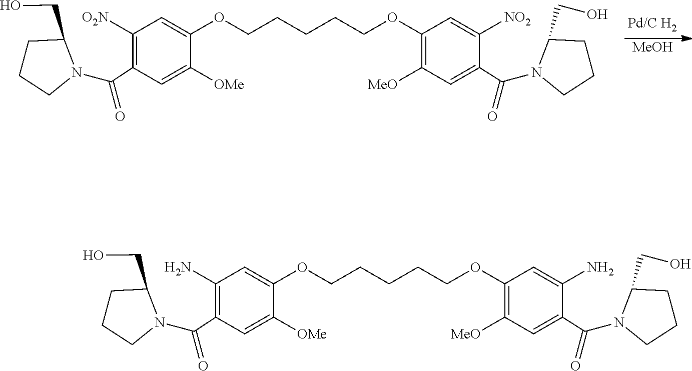 [SMILES: "MeOH" ; COc1cc(C(=O)N2CCC[C@H]2CO)c([N+](=O)[O-])cc1OCCCCCOc1cc([N+](=O)[O-])c(C(=O)N2CCC[C@H]2CO)cc1OC ; COc1cc(C(=O)N2CCC[C@H]2CO)c(N)cc1OCCCCCOc1cc(N)c(C(=O)N2CCC[C@H]2CO)cc1OC]

To a solution of (S)-((pentane-1,5-diylbis(oxy))bis(5-methoxy-2-nitro-4,1-phenylene))-bis(((S)-2-(hydroxymethyl)pyrrolidin-1-yl)methanone) (6.0 g, 9.1 mmol) in MeOH (100 mL) was added 10% Pd/C (2.4 g), the mixture was stirred under hydrogen atmosphere at room temperature overnight. After 14 h of stirring, the Pd/C was removed by filtration and washed with MeOH. The filtrate was concentrated and the residue was purified by chromatography (DCM/MeOH=10/1) to give (S)-((pentane-1,5-diylbis(oxy))bis(2-amino-5-methoxy-4,1-phenylene))bis(((S)-2-(hydroxymethyl)pyrrolidin-1-yl)methanone) (3.54 g, 5.9 mmol) as a white foam in 65% yield.

### Example 106. Synthesis of bis(4-((S)-2-((S)-2-(((allyloxy)carbonyl)amino)-3-methylbutanamido)propanamido)benzyl) ((S)-(pentane-1,5-diylbis(oxy))bis(2-((S)-2-(hydroxymethyl)pyrrolidine-1-carbonyl)-4-methoxy-5,1-phenylene))dicarbamate

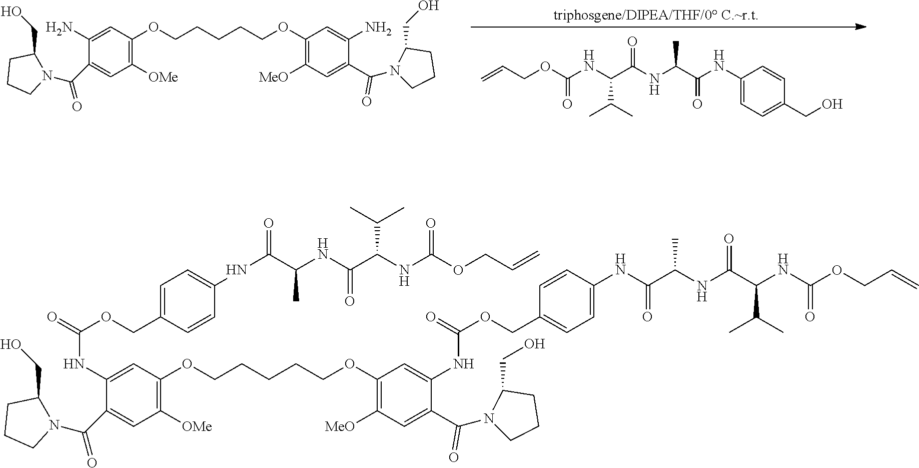 [SMILES: COc1cc(C(=O)N2CCC[C@H]2CO)c(N)cc1OCCCCCOc1cc(N)c(C(=O)N2CCC[C@H]2CO)cc1OC ; C=CCOC(=O)N[C@H](C(=O)N[C@@H](C)C(=O)Nc1ccc(CO)cc1)C(C)C ; C=CCOC(=O)N[C@H](C(=O)N[C@@H](C)C(=O)Nc1ccc(COC(=O)Nc2cc(OCCCCCOc3cc(NC(=O)OCc4ccc(NC(=O)[C@H](C)NC(=O)[C@@H](NC(=O)OCC=C)C(C)C)cc4)c(C(=O)N4CCC[C@H]4CO)cc3OC)c(OC)cc2C(=O)N2CCC[C@H]2CO)cc1)C(C)C]

To a solution of allyl ((S)-1-(((S)-1-((4-(hydroxymethyl)phenyl)amino)-1-oxopropan-2-yl)amino)-3-methyl-1-oxobutan-2-yl)carbamate (8.0 g, 21.3 mmol) in dry THF (300 mL) was added DIPEA (5.5 g, 40.3 mmol) and a solution of triphosgene (3.2 g, 10.8 mmol) in dry THF (50 mL) at 5° C. After 15 min of stirring, the solution was recooled to 5° C. and a mixture of (S)-((pentane-1,5-diylbis(oxy))bis(2-amino-5-methoxy-4,1-phenylene))bis (((S)-2-(hydroxymethyl)-pyrrolidin-1-yl)methanone) (3.2 g, 5.3 mmol) and DIPEA (2.75 g, 21.6 mmol) in dry THE (150 mL) was added. The resultant solution was allowed to warm to room temperature and stirred overnight. The THF removed by evaporation in vacuo. The residue was purified by chromatography (DCM/MeOH=20/1) to give bis(4-((S)-2-((S)-2-(((allyloxy)carbonyl)amino)-3-methylbutanamido) propanamido)-benzyl)((S)-(pentane-1,5-diylbis(oxy))bis(2-((S)-2-(hydroxymethyl)pyrrolidine-1-carbonyl)-4-methoxy-5,1-phenylene))dicarbamate (7.0 g, 4.97 mmol) as a yellow foam in 94% yield.

### Example 107. Synthesis of (11S,11aS,11′S,11a′S)-bis(4-((S)-2-((S)-2-(((allyloxy)carbonyl)-amino)-3-methylbutanamido)propanamido)benzyl) 8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate)

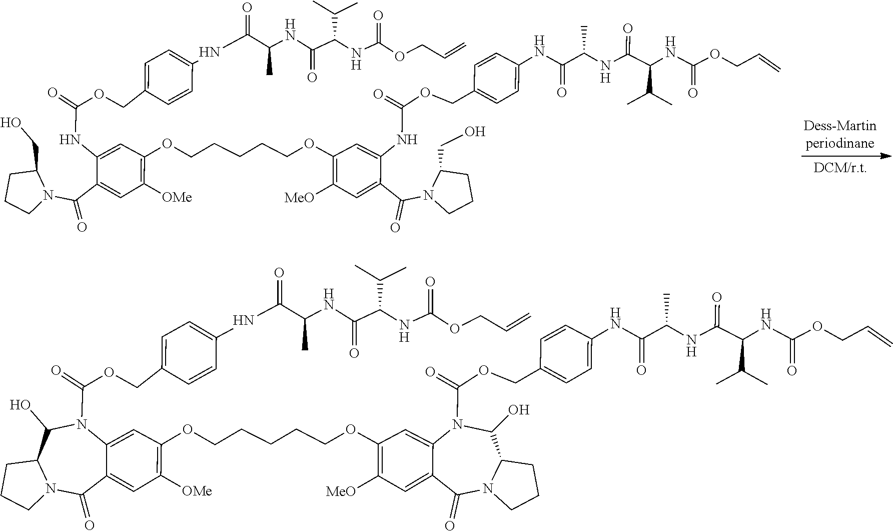

To a solution of bis(4-((S)-2-((S)-2-(((allyloxy)carbonyl)amino)-3-methyl butanamido) propanamido)benzyl)((S)-(pentane-1,5-diylbis(oxy))bis(2-((S)-2-(hydroxy-methyl)pyrrolidine-1-carbonyl)-4-methoxy-5,1-phenylene))dicarbamate (300 mg, 0.21 mmol) in dry DCM (15 mL) was added DMP (280 mg, 0.66 mmol) under nitrogen at room temperature. After completion of conversion, the reaction solution was added aqueous Na₂SO₃ and followed by aqueous NaHCO₃, the mixture was stirred for further 15 minutes and extracted with DCM (3×20 mL). The combined organic extract was washed with brine, dried, filtered and concentrated. The residue was purified by chromatography (DCM/MeOH=20/1) to give (11S,11aS,11′S,11a′S)-bis(4-((S)-2-((S)-2-(((allyloxy)carbonyl)amino)-3-methylbutanamido)propanamido)benzyl)8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate) (270 mg, 0.19 mmol) as a off-white foam in 92% yield.

### Example 108. Synthesis of (11S,11aS,11′S,11a′S)-bis(4-((S)-2-((S)-2-(((allyloxy)carbonyl)-amino)-3-methylbutanamido)propanamido)benzyl) 8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate)

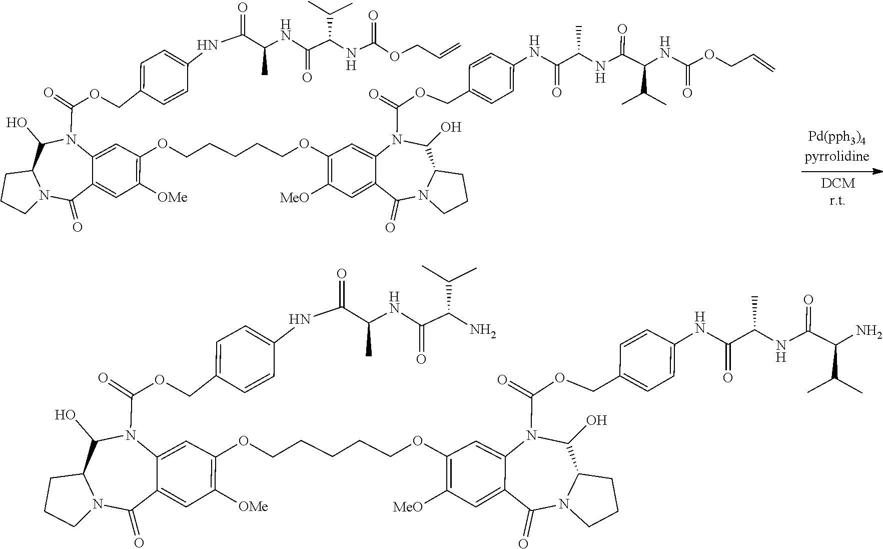

To a solution of (11S,11aS,11′S,11a′S)-bis(4-((S)-2-((S)-2-(((allyloxy)carbonyl) amino)-3-methylbutanamido)propanamido)benzyl)8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate) (774 mg, 0.55 mmol) and pyrrolidine (196 mg, 2.76 mmol) in dry DCM (8 mL) was added Pd(pph₃)₄ (76 mg, 0.066 mmol). The reaction was flushed with argon and stirred for 2 h at room temperature, after which the reaction was diluted with DCM and washed sequentially with saturated aqueous NH₄Cl and brine. The organic phase was dried over Na₂SO₄, filtered and concentrated. The residue was purified by chromatography (DCM/MeOH=6/1) to give (11S,11aS,11′S,11a′S)-bis(4-((S)-2-((S)-2-(((allyloxy) carbonyl)amino)-3-methylbutanamido)propanamido)benzyl)8,8′-(pentane-1,5-diylbis(oxy))bis(11-hydroxy-7-methoxy-5-oxo-2,3,11,11a-tetrahydro-1H-benzo[e]-pyrrolo[1,2-a][1,4]diazepine-10(5H)-carboxylate) (420 mg, 0.34 mmol) as an off-white solid in 62% yield.

### Example 109. Synthesis of (S)-2-(((allyloxy)carbonyl)amino)-3-methylbutanoic Acid

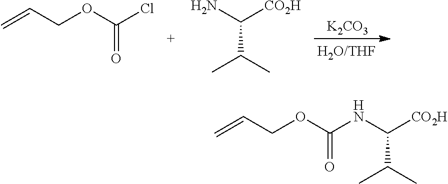

Allyl chloroformate (24.8 g, 205 mmol) was added dropwise to a stirred solution of L-valine (20 g, 171 mmol) and K₂CO₃ (35.4 g, 257 mmol) in H₂O (250 mL) and THF (250 mL). The reaction mixture was stirred at room temperature overnight, then the solvent was concentrated under reduced pressure and the remaining solution extracted with diethyl ether (100 mL). The aqueous portion was acidified to pH 2 with conc. HCl and extracted with DCM (3×200 mL). The combined organics were washed with brine, dried over Na₂SO₄, filtered and concentrated to afford the product (35 g, 174 mmol). White solid, yield (100%).

### Example 110. Synthesis of (S)-2,5-dioxopyrrolidin-1-yl 2-(((allyloxy)carbonyl)amino)-3-methylbutanoate

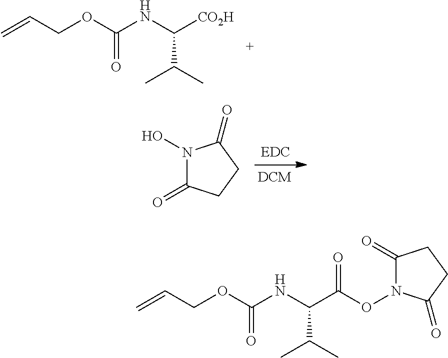

To a stirred solution of (S)-2-(((allyloxy)carbonyl)amino)-3-methylbutanoic acid (35 g, 174 mmol) in dry DCM (500 mL) was added EDC (66.9 g, 348 mmol) and N-hydroxysuccinimide (30 g, 261 mmol) at room temperature. After 14 h of stirring, the reaction was diluted with DCM and washed with water and brine. The organic phase was dried over Na₂SO₄, filtered and concentrated to afforded the product (54.5 g) which was used in the next step without further purification. Yield: (100%) viscous colourless oil. R_{f}=0.5 (PE/EA=2/1)

### Example 111. Synthesis of (S)-2-((S)-2-(((allyloxy)carbonyl)amino)-3-methylbutanamido)-propanoic Acid

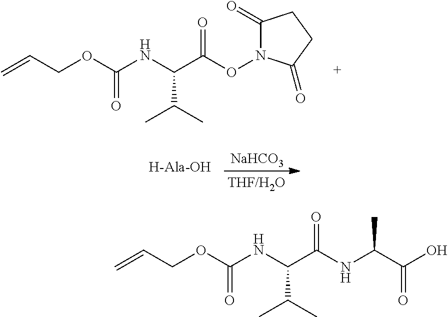

To a solution of H-Ala-OH (15.7 g, 176 mmol) and NaHCO₃(15.5 g, 185 mmol) in THF (200 mL) and H₂O (200 mL) was added a solution of (S)-2,5-dioxopyrrolidin-1-yl 2-(((allyloxy)-carbonyl)amino)-3-methylbutanoate (50 g, 168 mmol) in THF (100 mL) at room temperature. After 72 hours of stirring, the THF was evaporated under reduced pressure. The residue was acidified to pH 3 with citric acid and extracted with EA (3×350 mL), the combined extracts was washed with brine, dried, filtered and concentrated to give a white solid. Trituration with diethyl ether (excess) afforded the pure product as a white powder (25.2 g, 93 mmol, 55%).

### Example 112. Synthesis of Allyl ((S)-1-(((S)-1-((4-(hydroxymethyl)phenyl)amino)-1-oxopropan-2-yl)amino)-3-methyl-1-oxobutan-2-yl)carbamate

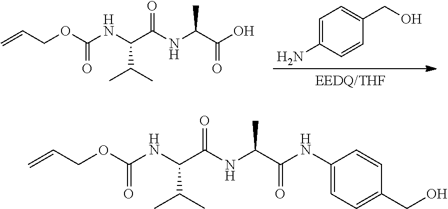 [SMILES: C=CCOC(=O)N[C@H](C(=O)N[C@@H](C)C(=O)O)C(C)C ; Nc1ccc(CO)cc1 ; C=CCOC(=O)N[C@H](C(=O)N[C@@H](C)C(=O)Nc1ccc(CO)cc1)C(C)C]

To a solution of (S)-2-((S)-2-(((allyloxy)carbonyl)amino)-3-methylbutanamido)-propanoic acid (25.2 g, 92.6 mmol) and p-aminobenzyl alcohol (12.0 g, 97.6 mmol) in THF (300 mL) was added EEDQ (24.0 g, 97.2 mmol) at room temperature. After 18 hours of stirring, the solvent was evaporated under reduced pressure to give a pale brown solid. The solid was triturated with diethyl ether and filtered, washing with an excess of diethyl ether. This afforded the product as a white solid (40 g, 106 mmol, 100%).

### Example 113. Synthesis of 4-(((benzyloxy)carbonyl)amino)butanoic Acid

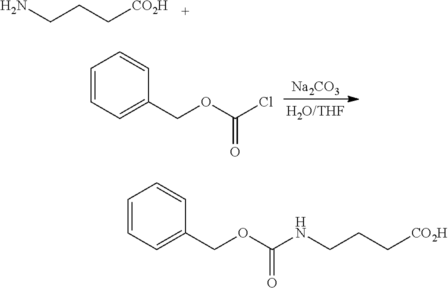

Na₂CO₃ (41.1 g, 387 mmol) was added to a solution of 4-aminobutanoic acid (20 g, 193 mmol) in H₂O (300 mL) at 5° C. After 10 min of stirring, a solution of CbzCl (33.2 mL, 232 mmol) in THF (100 mL) was added dropwise. The reaction was allowed to warm to room temperature and stirred overnight. After completion of conversion, the mixture was diluted with H₂O (100 mL) and extracted with EA (2×100 mL). The aqueous layer was acidified to pH 2 with conc. HCl and extracted with EA (3×100 mL). The combined organics were washed with brine, dried over Na₂SO₄, filtered and concentrated to give a white solid. Trituration with PE (excess) afforded the pure product as a white powder (31.6 g, 70%).

### Example 114. Synthesis of Tert-butyl 4-(((benzyloxy)carbonyl)amino)butanoate

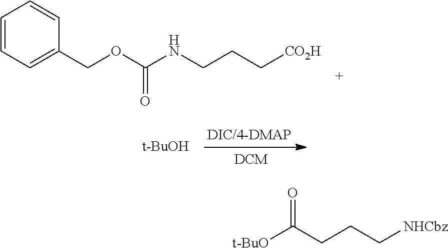 [SMILES: "t-BuOH" ; O=C(O)CCCNC(=O)OCc1ccccc1 ; CC(C)(C)OC(=O)CCCNC(=O)OCc1ccccc1]

To a stirred solution of 4-(((benzyloxy)carbonyl)amino)butanoic acid (5.9 g, 24.9 mmol) and tert-Butanol (14.7 g, 199 mmol) in dry DCM (250 mL) was added 4-DMAP (0.61 g, 5 mmol) and DIC (4.7 g, 37.3 mmol) at 0° C. After 16 h of stirring, the reaction was filtered and extracted with DCM (2×200 mL). The combined organic extract was washed with 1N HCl and brine, dried over Na₂SO₄, filtered and concentrated. The residue was purified by chromatography (100% DCM) to give tert-butyl 4-(((benzyloxy)carbonyl)amino)butanoate (4.26 g, 14.5 mmol, 58%) viscous colourless oil.

### Example 115. Synthesis of Tert-butyl 4-aminobutanoate

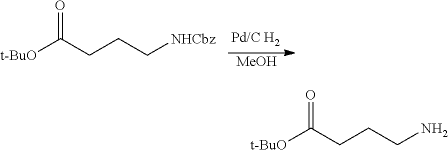 [SMILES: "MeOH" ; CC(C)(C)OC(=O)CCCNC(=O)OCc1ccccc1 ; CC(C)(C)OC(=O)CCCN]

To a solution of tert-butyl 4-(((benzyloxy)carbonyl)amino)butanoate (1.69 g, 5.77 mmol) in MeOH (40 mL) was added 10% Pd/C (400 mg), the mixture was stirred under hydrogen atmosphere at room temperature overnight. After 14 h of stirring, the Pd/C was removed by filtration and washed with MeOH. The filtrate was concentrated to afford the product which was used in the next step without further purification (897 mg, 5.64 mmol). colorless liquid, yield (98%).

### Example 116. Synthesis of (2R,3S)-2,3-bis(benzylamino)succinic Acid

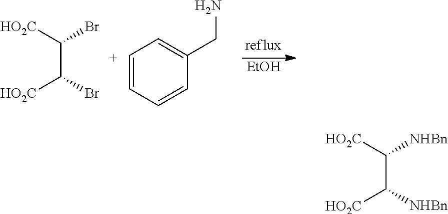

To a solution of meso-2,3-dibromosuccinic acid (50 g, 181 mmol) in EtOH (400 mL) was added benzylamine (150 mL) dropwise. After completion of addition, the mixture was heated to 90° C. and stirred overnight. The mixture was cooled to room temperature and diluted with H₂O. 6N HCl was added until pH 4 to give white precipitates. The precipitates were filtered, rinsed with H₂O and dried to give (2R,3S)-2,3-bis(benzylamino)succinic acid (50 g, 152 mmol, 84%).

### Example 117. Synthesis of (2R,3S)-2,3-diaminosuccinic Acid

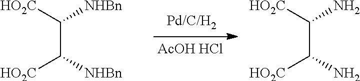

To a solution of (2R,3S)-2,3-bis(benzylamino)succinic acid (18 g, 55 mmol) in AcOH (100 mL) and HCl (100 mL) was added 10% Pd/C (3 g), the mixture was stirred under hydrogen atmosphere at 50° C. overnight. After 48 h of stirring, the Pd/C was removed by filtration and washed with H₂O. The filtrate was concentrated and the residue was dissolved in 1N NaOH (200 mL). AcOH was added until pH 5 to give white precipitates. The precipitates were filtered, rinsed with H₂O and dried to give (2R,3S)-2,3-diaminosuccinic acid (8.7 g, 58.8 g, 100%).

### Example 118. Synthesis of 2,3-bis(((benzyloxy)carbonyl)amino)succinic Acid

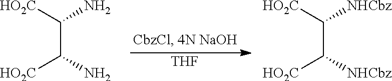 [SMILES: N[C@H](C(=O)O)[C@@H](N)C(=O)O ; O=C(N[C@H](C(=O)O)[C@@H](NC(=O)OCc1ccccc1)C(=O)O)OCc1ccccc1 ; "THF"]

To a solution of (2R,3S)-2,3-diaminosuccinic acid (31.74 g, 214 mmol) in THF (220 mL) and 4N NaOH (214 mL) was added CbzCl (61 mL, 428 mmol) dropwise at 0° C. After completion of addition, the mixture was allowed to warm to room temperature and stirred for 2 h. The reaction was diluted with H₂O (1600 mL) and extracted with EA (2×15600 mL). The aqueous layer was separated and acidified with conc. HCl until pH 2 was reached. The resultant solution was stirred for 1 h and stranded at 5° C. to give white precipitates. The precipitates were filtered, rinsed with H₂O and dried to give 2,3-bis(((benzyloxy)carbonyl)amino)succinic acid (52.2 g, 125 mmol, 59%).

### Example 119. Synthesis of Dibenzyl ((3R,4S)-2,5-dioxotetrahydrofuran-3,4-diyl)dicarbamate

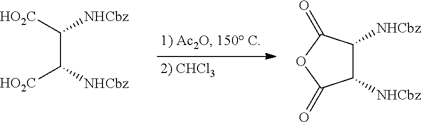 [SMILES: O=C(N[C@H](C(=O)O)[C@@H](NC(=O)OCc1ccccc1)C(=O)O)OCc1ccccc1 ; O=C(N[C@@H]1C(=O)OC(=O)[C@@H]1NC(=O)OCc1ccccc1)OCc1ccccc1]

The solution of 2,3-bis(((benzyloxy)carbonyl)amino)succinic acid (5.0 g, 12 mmol) in Ac₂O (37.5 mL) was refluxed for 20 min, cooled and concentrated to give resulting anhydride. The diastereomeric mixture was treat with CHCl₃ (37 mL), the insoluble meso-isomer was filtered and washed with PE to give crystals of dibenzyl ((3R,4S)-2,5-dioxotetrahydrofuran-3,4-diyl)dicarbamate (2.0 g, 5 mmol, 42%)

### Example 120. Synthesis of Di-tert-butyl 4,4′-(((2R,3S)-2,3-bis(((benzyloxy)carbonyl)-amino)succinyl)bis(azanediyl))dibutanoate

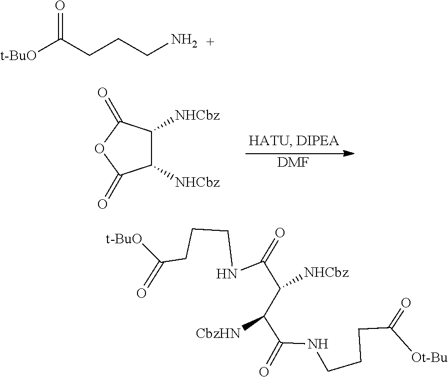

To a solution of dibenzyl ((3R,4S)-2,5-dioxotetrahydrofuran-3,4-diyl)dicarbamate (2.03 g, 5.1 mmol) and tert-butyl 4-aminobutanoate (1.79 g, 11.3 mmol) in DMF (45 mL) was added DIPEA (1.98 g, 15.3 mmol) at 0° C. After 5 min of stirring, HATU (4.66 g, 12.3 mmol) was added. The mixture was allowed to warm to room temperature and stirred for 2 h. After completion of conversion, the mixture was diluted with H₂O (90 mL) and extracted with EA (2×200 mL) and DCM (2×90 mL), the combined organic extract was washed with brine and dried over Na₂SO₄. The majority of solvent was removed under reduced pressure and a white solid was precipitated, which was collected and dried to give di-tert-butyl 4,4′-(((2R,3S)-2,3-bis (((benzyloxy)carbonyl)amino)succinyl)bis(azanediyl))dibutanoate (2.8 g, 4.0 mmol) as a white solid in 80% yield.

### Example 121. Synthesis of Di-tert-butyl 4,4′-(((2R,3S)-2,3-diaminosuccinyl)bis-(azanediyl))dibutanoate

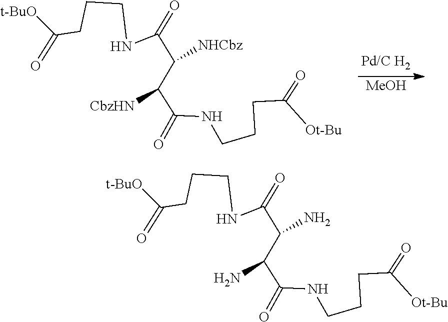

To a solution of 4,4′-(((2R,3S)-2,3-bis(((benzyloxy)carbonyl)amino)succinyl)bis-(azanediyl))dibutanoate (2.8 g, 4.0 mmol) in MeOH (100 mL) was added 10% Pd/C (1.1 g), the mixture was stirred under hydrogen atmosphere at room temperature overnight. After 18 h of stirring, the Pd/C was removed by filtration and washed with MeOH. The filtrate was concentrated to afford the product which was used in the next step without further purification (940 mg, 2.2 mmol). colorless liquid, yield (55%).

### Example 122. Synthesis of Di-tert-butyl 4,4′-(((2R,3S)-2,3-bis(4-(2,5-dioxo-2,5-dihydro-1H-pyrrol-1-yl)butanamido)succinyl)bis(azanediyl))dibutanoate

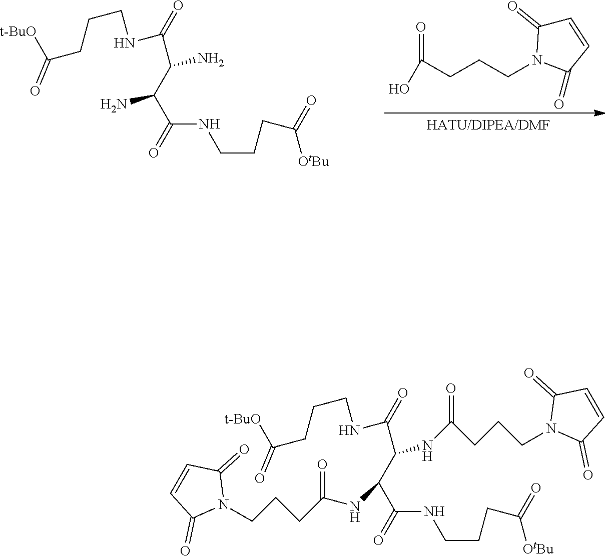 [SMILES: CC(C)(C)OC(=O)CCCNC(=O)[C@@H](N)[C@@H](N)C(=O)NCCCC(=O)OC(C)(C)C ; O=C(O)CCCN1C(=O)C=CC1=O ; CC(C)(C)OC(=O)CCCNC(=O)[C@@H](NC(=O)CCCN1C(=O)C=CC1=O)[C@@H](NC(=O)CCCN1C(=O)C=CC1=O)C(=O)NCCCC(=O)OC(C)(C)C]

To a solution of di-tert-butyl 4,4′-(((2R,3S)-2,3-diaminosuccinyl)bis(azanediyl))-dibutanoate (940 mg, 2.19 mmol) and 4-(2,5-dioxo-2,5-dihydro-1H-pyrrol-1-yl)butanoic acid (840 mg, 4.59 mmol) in DMF (25 mL) was added DIPEA (1.13 g, 8.76 mmol) at 0° C. After 5 min of stirring, HATU (1.74 g, 4.58 mmol) was added. The mixture was allowed to warm to room temperature and stirred for 1 h. After completion of conversion, the mixture was diluted with H₂O (50 mL) and extracted with EA (2×100 mL) and DCM (2×50 mL), the combined organic extracts were washed with brine and dried over Na₂SO₄. The majority of solvent was removed under reduced pressure and a white solid was precipitated, which was collected and dried to give di-tert-butyl 4,4′-(((2R,3S)-2,3-bis(4-(2,5-dioxo-2,5-dihydro-1H-pyrrol-1-yl)butanamido)succinyl)bis-(azanediyl))dibutanoate (1.36 g, 1.79 mmol) as a white solid in 82% yield.

### Example 123. Synthesis of 4,4′-(((2R,3S)-2,3-bis(4-(2,5-dioxo-2,5-dihydro-1H-pyrrol-1-yl)butanamido)succinyl) bis(azanediyl))dibutanoic Acid

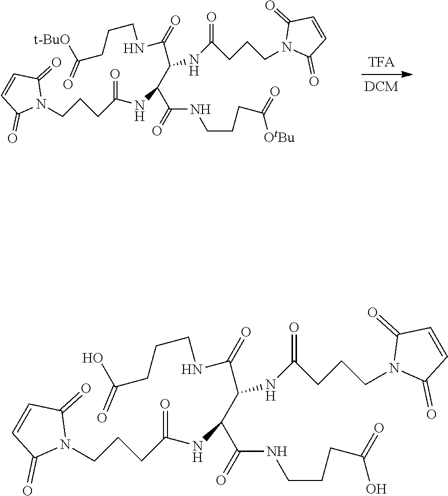 [SMILES: CC(C)(C)OC(=O)CCCNC(=O)[C@@H](NC(=O)CCCN1C(=O)C=CC1=O)[C@@H](NC(=O)CCCN1C(=O)C=CC1=O)C(=O)NCCCC(=O)OC(C)(C)C ; O=C(O)CCCNC(=O)[C@@H](NC(=O)CCCN1C(=O)C=CC1=O)[C@@H](NC(=O)CCCN1C(=O)C=CC1=O)C(=O)NCCCC(=O)O]

To a solution of di-tert-butyl 4,4′-(((2R,3S)-2,3-bis(4-(2,5-dioxo-2,5-dihydro-1H-pyrrol-1-yl)butanamido)succinyl)bis(azanediyl))dibutanoate (1.36 g, 1.79 mmol) in DCM (15 mL) was added TFA (30 mL) at room temperature 0° C. After 18 h of stirring, the reaction was concentrated and the residue was dissolved in dry toluene. The solvent was removed by evaporation in vacuo to give white precipitates which was used in the next step without further purification (1.3 mg, 2.0 mmol). yield (100%).

### Example 124. Synthesis of PBD Product C-7

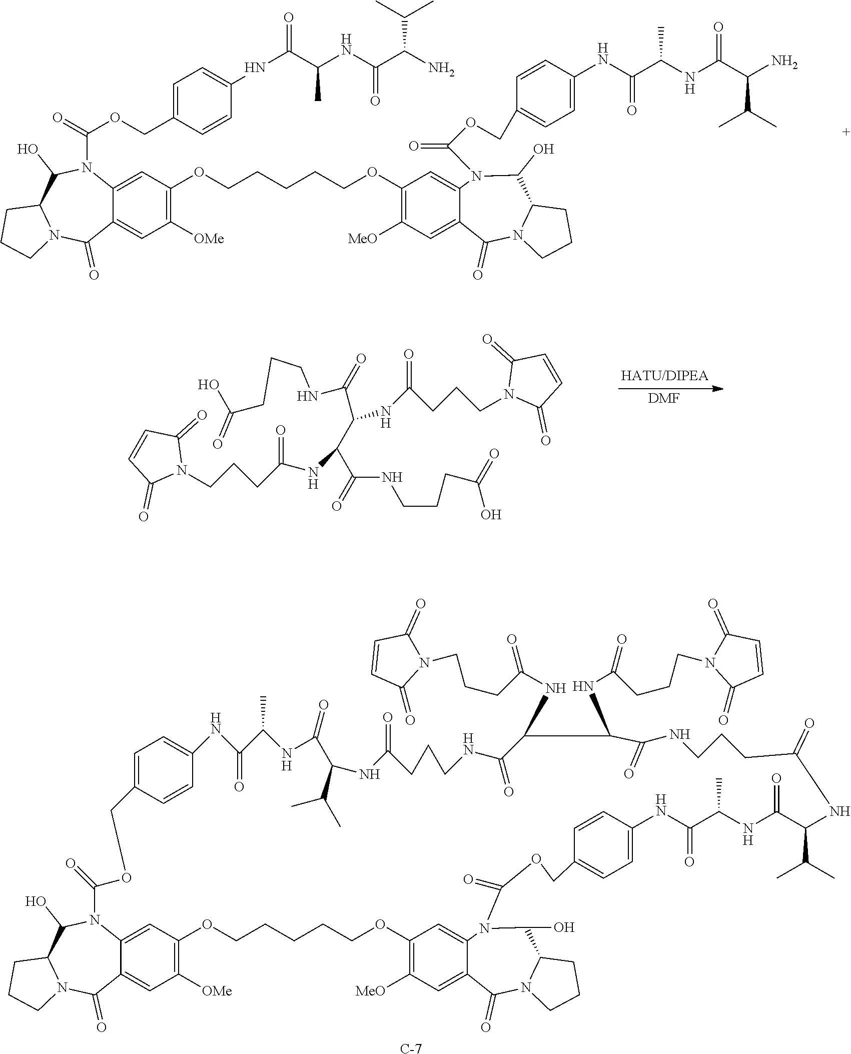

To a solution of (11S,11aS,11′S,11a′S)-bis(4-((S)-2-((S)-2-(((allyloxy)carbonyl) amino)-3-methylbutanamido)propanamido)benzyl)8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate) (215 mg, 0.17 mmol) and 4,4′-(((2R,3S)-2,3-bis(4-(2,5-dioxo-2,5-dihydro-1H-pyrrol-1-yl)butanamido)succinyl)bis(azanediyl))dibutanoic acid (115 mg, 0.18 mmol) in DMF (18 mL) was added DIPEA (90 mg, 0.70 mmol) at 0° C. After 5 min of stirring, HATU (132 mg, 0.35 mmol) was added. The mixture was allowed to warm to room temperature and stirred overnight. After completion of conversion, the mixture was diluted with H₂O (2 mL) and extracted with EA (2×40 mL) and DCM (2×20 mL), the combined organic extract was washed with brine, dried, filtered and concentrated. The residue was purified by pre-HPLC to give PBD product C-6 (10 mg) as a white powder.

### Example 125. Synthesis of Di-tert-butyl 4,4′-(((2R,3S)-2,3-bis(4-(2,5-dioxo-2,5-dihydro-1H-pyrrol-1-yl)butanamido)succinyl)bis(azanediyl))dibutanoate

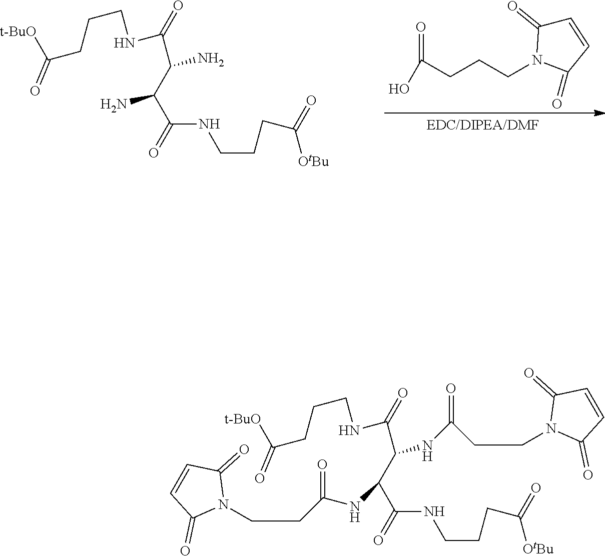 [SMILES: CC(C)(C)OC(=O)CCCNC(=O)[C@@H](N)[C@@H](N)C(=O)NCCCC(=O)OC(C)(C)C ; O=C(O)CCCN1C(=O)C=CC1=O ; CC(C)(C)OC(=O)CCCNC(=O)[C@@H](NC(=O)CCN1C(=O)C=CC1=O)[C@@H](NC(=O)CCN1C(=O)C=CC1=O)C(=O)NCCCC(=O)OC(C)(C)C]

To a solution of di-tert-butyl 4,4′-(((2R,3S)-2,3-diaminosuccinyl)bis(azanediyl))-dibutanoate (900 mg, 2.09 mmol) and 3-(2,5-dioxo-2,5-dihydro-1H-pyrrol-1-yl)propanoic acid (840 mg, 4.97 mmol) in DMF (25 mL) was added DIPEA (0.93 g, 7.21 mmol) at 0° C. After 5 min of stirring, EDC (1.74 g, 9.06 mmol) was added. The mixture was allowed to warm to room temperature and stirred for 1 h. After completion of conversion, the mixture was diluted with H₂O (50 mL) and extracted with EA (2×100 mL) and DCM (2×50 mL), the combined organic extracts were washed with brine and dried over Na₂SO₄. The majority of solvent was removed under reduced pressure and a white solid was precipitated, which was collected and dried to give di-tert-butyl 4,4′-(((2R,3S)-2,3-bis(3-(2,5-dioxo-2,5-dihydro-1H-pyrrol-1-yl)propanamido)succinyl)bis-(azanediyl))dibutanoate (1.27 g, 1.79 mmol) as a white solid in 83% yield. ESI MS m/z+ C₃₄H₄₉N₆O₁₂, cacld. 733.33 (M+H), found 733.55.

### Example 126. Synthesis of 4,4′-(((2R,3S)-2,3-bis(3-(2,5-dioxo-2,5-dihydro-1H-pyrrol-1-yl)propanamido)succinyl)bis(azanediyl))dibutanoic Acid

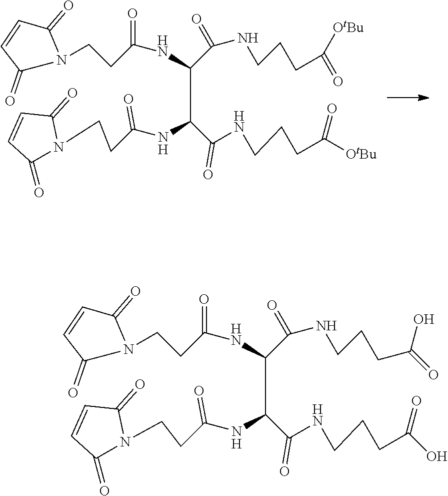 [SMILES: CC(C)(C)OC(=O)CCCNC(=O)[C@@H](NC(=O)CCN1C(=O)C=CC1=O)[C@@H](NC(=O)CCN1C(=O)C=CC1=O)C(=O)NCCCC(=O)OC(C)(C)C ; O=C(O)CCCNC(=O)[C@@H](NC(=O)CCN1C(=O)C=CC1=O)[C@@H](NC(=O)CCN1C(=O)C=CC1=O)C(=O)NCCCC(=O)O]

Di-tert-butyl 4,4′-(((2R,3S)-2,3-bis(4-(2,5-dioxo-2,5-dihydro-1H-pyrrol-1-yl)butanamido)succinyl)bis(azanediyl))dibutanoate (502.0 mg, 0.685 mmol) in 1,4-dioxane (8 ml) at 4° C. was added conc. HCl (3 ml). The mixture was then stirred at RT for 30 min, diluted with 1,4-dioxane (8 ml), concentrated, co-evaporated with dioxane/toluene (1:1, 2×10 ml) to dryness and crystallized with EtOH/Hexane to afford the title compound (289.0 g, 68% yield). ESI MS m/z+ C₂₆H₃₃N₆O₁₂, cacld. 621.21 (M+H), found 621.55.

### Example 127. Synthesis of Allyl ((S)-3-methyl-1-(((S)-1-((4-((((4-nitrophenoxy)carbonyl)-oxy)methyl)phenyl)amino)-1-oxopropan-2-yl)amino)-1-oxobutan-2-yl)carbamate

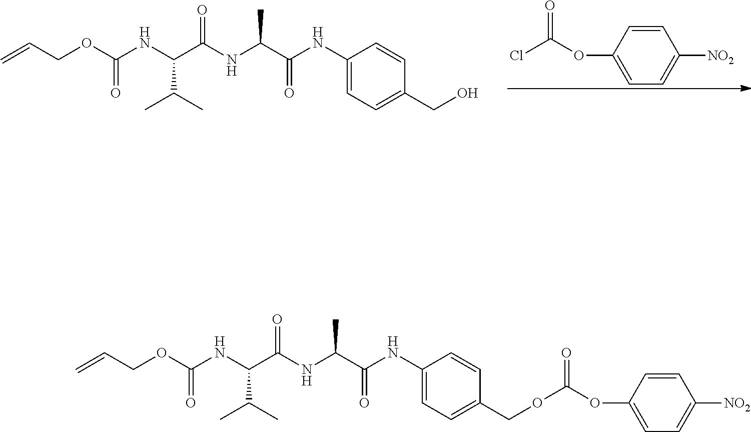 [SMILES: C=CCOC(=O)N[C@H](C(=O)N[C@@H](C)C(=O)Nc1ccc(CO)cc1)C(C)C ; O=C(Cl)Oc1ccc([N+](=O)[O-])cc1 ; C=CCOC(=O)N[C@H](C(=O)N[C@@H](C)C(=O)Nc1ccc(COC(=O)Oc2ccc([N+](=O)[O-])cc2)cc1)C(C)C]

Allyl((S)-1-(((S)-1-((4-(hydroxymethyl)phenyl)amino)-1-oxopropan-2-yl)amino)-3-methyl-1-oxobutan-2-yl)carbamate (2.21 g, 5.86 mmol) in the mixture of dry pyridine (5 ml) and CH₂Cl₂ (20 ml) was added 4-nitrophenyl carbonochloridate (1.82 g, 9.05 mmol). The mixture was stirred at RT for 8 hour, concentrated and purified on SiO₂ column eluted with EtOAc/CH₂Cl₂ (1:12) to afford the title compound (2.63 g, 83% yield). MS ESI m/z calcd for C₂₆H₃₁N₄O₉ [M+H]⁺ 543.21, found 543.60.

### Example 128. Synthesis of (11aS,11a′S)-bis(4-((S)-2-((S)-2-(((allyloxy)carbonyl)amino)-3-methylbutanamido)propanamido)benzyl) 8,8′-(pentane-1,5-diylbis(oxy))bis(7-methoxy-2-methylene-5-oxo-2,3,11,11a-tetrahydro-1H-benzo[e]pyrrolo[1,2-a][1,4]diazepine-10(5H)-carboxylate)

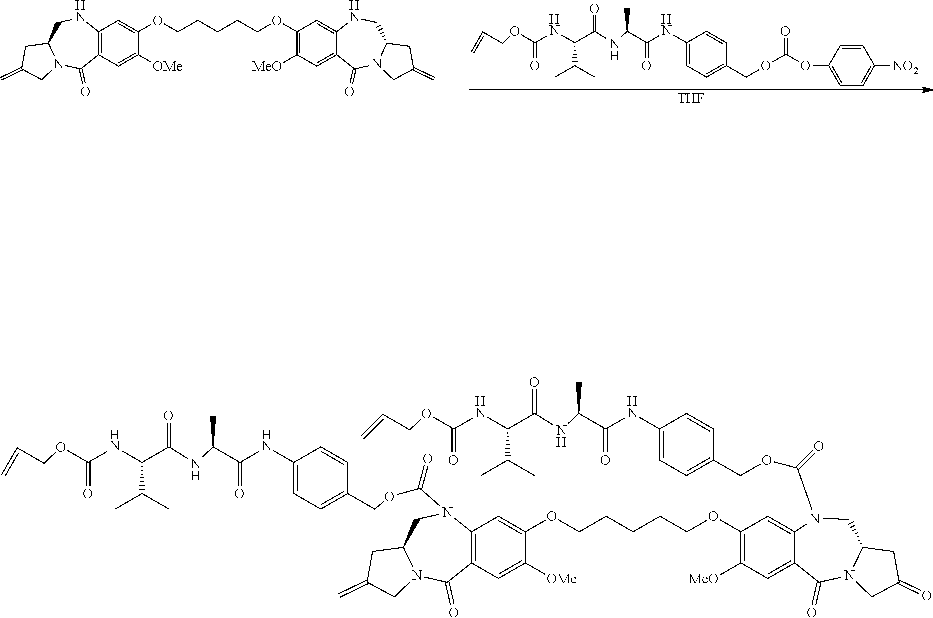

(11aS,11a′S)-8,8′-(Pentane-1,5-diylbis(oxy))bis(7-methoxy-2-methylene-2,3,11,11a-tetrahydro-1H-benzo[e]pyrrolo[1,2-a][1,4]diazepin-5(10H)-one) (288.2 mg, 0.490 mmol) in dry CH₃CN (5 ml) was added allyl ((S)-3-methyl-1-(((S)-1-((4-((((4-nitrophenoxy)carbonyl)oxy)-methyl)phenyl)amino)-1-oxopropan-2-yl)amino)-1-oxobutan-2-yl)carbamate (770.2 mg, 1.420 mmol) and DIPEA (2 ml). The mixture was stirred at 45° C. for 8 h, concentrated and purified on SiO₂ column eluted with EtOAc/CH₂Cl₂ (1:8) to afford the title compound (492.0 mg, 72% yield). MS ESI m/z calcd for C₇₃H₉₁N₁₀O₁₈ [M+H]⁺ 1395.64, found 1395.95.

### Example 129. Synthesis of (11aS,11a′S)-bis(4-((S)-2-((S)-2-amino-3-methylbutanamido)-propanamido)benzyl) 8,8′-(pentane-1,5-diylbis(oxy))bis(7-methoxy-2-methylene-5-oxo-2,3,11,11a-tetrahydro-1H-benzo[e]pyrrolo[1,2-a][1,4]diazepine-10(5H)-carboxylate)

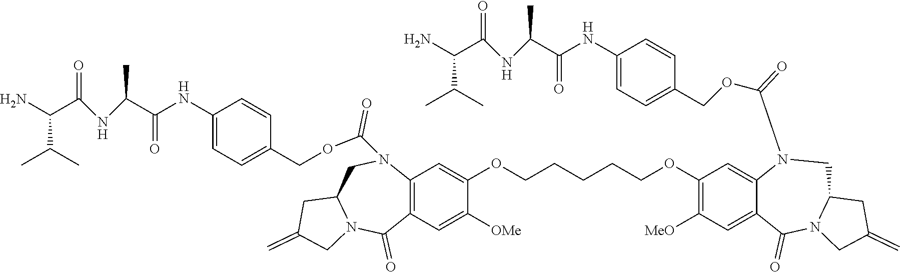 [SMILES: C=C1C[C@H]2CN(C(=O)OCc3ccc(NC(=O)[C@H](C)NC(=O)[C@@H](N)C(C)C)cc3)c3cc(OCCCCCOc4cc5c(cc4OC)C(=O)N4CC(=C)C[C@H]4CN5C(=O)OCc4ccc(NC(=O)[C@H](C)NC(=O)[C@@H](N)C(C)C)cc4)c(OC)cc3C(=O)N2C1]

To a solution of (11aS,11a′S)-bis(4-((S)-2-((S)-2-(((allyloxy)carbonyl)amino)-3-methylbutanamido)propanamido)benzyl) 8,8′-(pentane-1,5-diylbis(oxy))bis(7-methoxy-2-methylene-5-oxo-2,3,11,11a-tetrahydro-1H-benzo[e]pyrrolo[1,2-a][1,4]diazepine-10(5H)-carboxylate) (274.2 mg, 0.197 mmol) and pyrrolidine (49 mg, 6.90 mmol) in dry DCM (5 mL) was added Pd(pph₃)₄ (152.0 mg, 0.132 mmol). The reaction was flushed with argon and stirred for 2 h at room temperature, after which the reaction was diluted with DCM and washed sequentially with saturated aqueous NH₄Cl and brine. The organic phase was dried over Na₂SO₄, filtered and concentrated. The residue was purified by chromatography (DCM/MeOH/Et₃N=6/1/0.02) to give the title compound (166.7 mg, 69% yield) as an off-white solid. MS ESI m/z calcd for C₆₅H₈₃N₁₀O₁₄ [M+H]⁺ 1227.60, found 1227.93.

### Example 130. Synthesis of PBD Product C-8

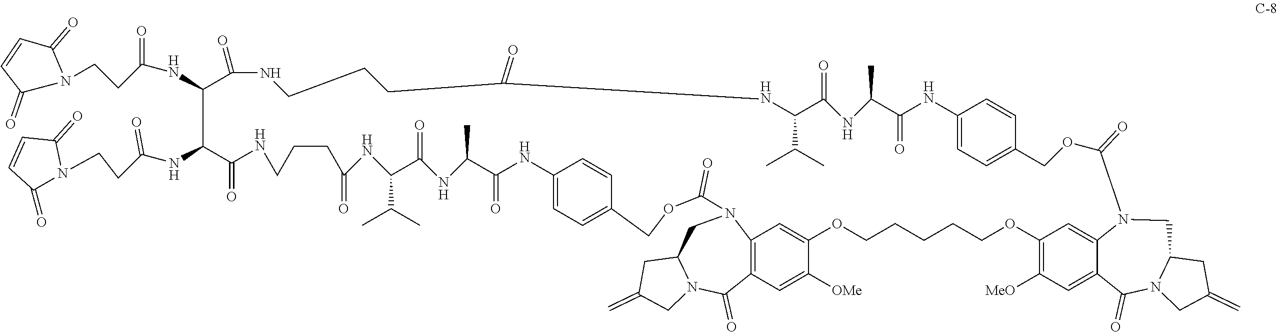 [SMILES: C=C1C[C@H]2CN3C(=O)OCc4ccc(cc4)NC(=O)[C@H](C)NC(=O)[C@H](C(C)C)NC(=O)CCCNC(=O)[C@@H](NC(=O)CCN4C(=O)C=CC4=O)[C@@H](NC(=O)CCN4C(=O)C=CC4=O)C(=O)NCCCC(=O)N[C@@H](C(C)C)C(=O)N[C@@H](C)C(=O)Nc4ccc(cc4)COC(=O)N4C[C@@H]5CC(=C)CN5C(=O)c5cc(OC)c(cc54)OCCCCCOc4cc3c(cc4OC)C(=O)N2C1]

(11aS,11a′S)-Bis(4-((S)-2-((S)-2-amino-3-methylbutanamido)-propanamido)benzyl) 8,8′-(pentane-1,5-diylbis(oxy))bis(7-methoxy-2-methylene-5-oxo-2,3,11,11a-tetrahydro-1H-benzo[e]pyrrolo[1,2-a][1,4]diazepine-10(5H)-carboxylate) (151.1 mg, 0.123 mmol) and 4,4′-(((2R,3S)-2,3-bis(3-(2,5-dioxo-2,5-dihydro-1H-pyrrol-1-yl)propanamido)succinyl)bis-(azanediyl))dibutanoic acid (77.1 mg, 0.124 mmol) in DMA (5 ml) was added EDC (95.2 mg, 0.496 mmol). The mixture was stirred at RT for 8 h, concentrated and purified on C-18 HPLC C18 3 μm column (25×4 cm) using gradient elution with a mixture of (A) acetonitrile and (B) water/0.1% formic acid (gradient: 15% A: 85% B up to 25% A: 75% B over 5 minutes, 35% A: 65% B for 15 minutes, 60% A: 40% B down to 50% A: 50% B over 15 minute, 15% A: 85% B for 5 minutes) with a 8 mL/minute flow rate. The fractions containing the title compound were pooled, evaporated and dried in a desiccator with P205 to afford the C-8 PBD compound (149.2 mg, 67% yield). MS ESI m/z calcd for C₉₁H₁₁₁N₁₆O₂₄ [M+H]⁺ 1811.79, found 1812.35.

### Example 131. Synthesis of (S)-(4-(benzyloxy)-5-methoxy-2-nitrophenyl)(2-(hydroxyl-ethyl)pyrrolidin-1-yl)methanone

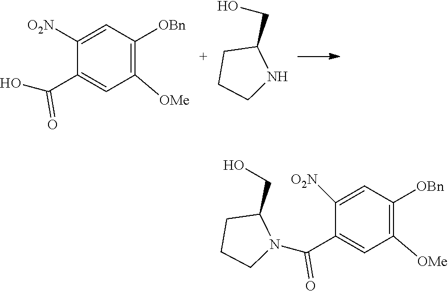 [SMILES: COc1cc(C(=O)O)c([N+](=O)[O-])cc1OCc1ccccc1 ; OC[C@@H]1CCCN1 ; COc1cc(C(=O)N2CCC[C@H]2CO)c([N+](=O)[O-])cc1OCc1ccccc1]

4-(benzyloxy)-5-methoxy-2-nitrobenzoic acid (10.20 g, 33.65 mmol) and (S)-pyrrolidin-2-ylmethanol (3.85 g, 38.09 mmol) in dry DMF (150 ml) was added EDC (19.50 g, 101.56 mmol). The mixture was stirred at RT for overnight, concentrated and purified on SiO₂ column eluted with EtOAc/CH₂Cl₂ (1:4) to afford the title compound (11.56 g, 89% yield). MS ESI m/z calcd for C₂₀H₂₃N₂O₆ [M+H]+ 387.15, found 387.65.

### Example 132. Synthesis of(S)-1-(4-(benzyloxy)-5-methoxy-2-nitrobenzoyl)pyrrolidine-2-carbaldehyde

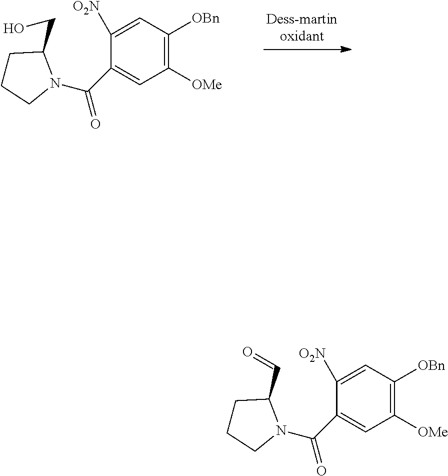 [SMILES: COc1cc(C(=O)N2CCC[C@H]2CO)c([N+](=O)[O-])cc1OCc1ccccc1 ; COc1cc(C(=O)N2CCC[C@H]2C=O)c([N+](=O)[O-])cc1OCc1ccccc1]

To a solution of (S)-(4-(benzyloxy)-5-methoxy-2-nitrophenyl)(2-(hydroxymethyl)-pyrrolidin-1-yl)methanone (3.80 g, 9.84 mmol) in dry DCM (15 mL) was added Dess-Martin periodinane (DMP) (5.80 g, 13.67 mmol) under nitrogen at room temperature. After completion of conversion, the reaction solution was added aqueous Na₂SO₃ and followed by aqueous NaHCO₃, the mixture was stirred for further 15 minutes and extracted with DCM (3×20 mL). The combined organic extract was washed with brine, dried, filtered and concentrated. The residue was purified by SiO₂ chromatography (DCM/EtOAc=4/1) to give the title compound (3.13 g, 83% yield) as an off-white foam. MS ESI m/z calcd for C₂₀H₂₁N₂O₆ [M+H]⁺ 385.13, found 385.60, 404.75 [M+H₂O+H]⁺.

### Example 133. Synthesis of 8-hydroxy-7-methoxy-2,3,11,11a-tetrahydro-1H-benzo[e]pyrrolo[1,2-a][1,4]diazepin-5(10H)-one

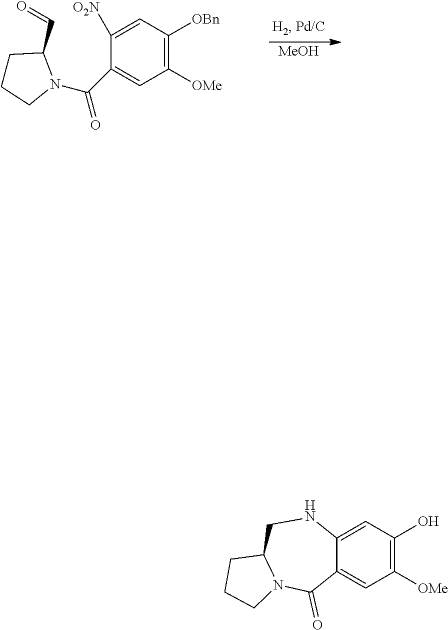 [SMILES: "MeOH" ; COc1cc(C(=O)N2CCC[C@H]2C=O)c([N+](=O)[O-])cc1OCc1ccccc1 ; COc1cc2c(cc1O)NC[C@@H]1CCCN1C2=O]

To a solution of S)-1-(4-(benzyloxy)-5-methoxy-2-nitrobenzoyl)pyrrolidine-2-carbaldehyde (3.00 g, 7.80 mmol) in methanol (75 mL) was Pd/C (10% Pd, 50% wet, 250 mg) in a hydrogenation shaker. After air in the shaker was vacuumed out, hydrogen (5 Psi) was conducted in. The reaction vessel was shaked overnight and filtered through Celite. The filtrant was concentrated and purified by SiO₂ chromatography (DCM/MeOH/Et₃N=4/1/0.05) to give the title compound (1.66 g, 86% yield) as a off-white foam. MS ESI m/z calcd for C₁₃H₁₇N₂O₃ [M+H]⁺ 249.12, found 249.50.

### Example 134. Synthesis of 4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl 8-hydroxy-7-methoxy-5-oxo-2,3,11,11a-tetrahydro-1H-benzo[e]pyrrolo[1,2-a][1,4]diazepine-10(5H)-carboxylate

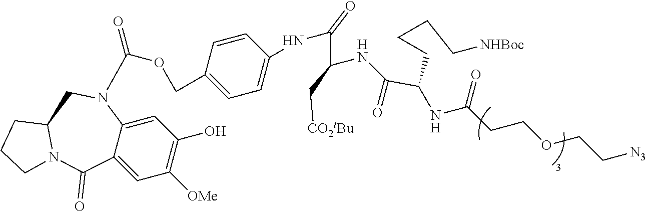

To a solution of (14S,17S)-tert-butyl 1-azido-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate (10.15 g, 13.50 mmol) in dry THF (300 mL) was added DIPEA (3.15 g, 24.41 mmol) and a solution of triphosgene (5.15 g, 17.36 mmol) in dry THF (50 mL) at 4-8° C. After 15 min of stirring, the solution was recooled to 4-8° C. and then added dropwise to a solution of 8-hydroxy-7-methoxy-2,3,11,11a-tetrahydro-1H-benzo[e]pyrrolo[1,2-a][1,4]diazepin-5(10H)-one (2.92 g, 11.76 mmol) in mixture of THF (100 mL) at 4-8° C. in 45 min. The resultant solution was allowed to warm to room temperature and stirred overnight. The mixture was diluted with toluene (50 ml), evaporated in vacuo and purified by SiO₂ chromatography (DCM/MeOH=15/1) to give the title compound (10.02 g, 82% yield) as a yellow foam. MS ESI m/z calcd for C₅₀H₇₄N₉O₁₅ [M+H]+ 1040.52, found 1040.90.

### Example 135. Synthesis of(S)-4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl 8-(3-iodopropoxy)-7-methoxy-5-oxo-2,3,11,11a-tetrahydro-1H-benzo[e]pyrrolo[1,2-a][1,4]diazepine-10(5H)-carboxylate

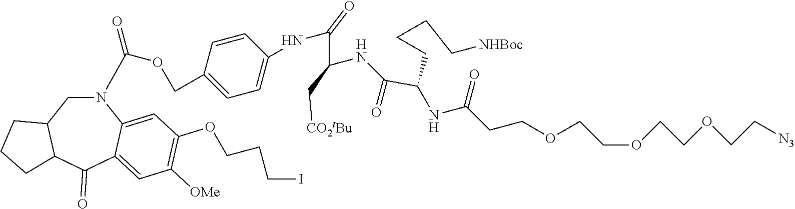 [SMILES: COc1cc2c(cc1OCCCI)N(C(=O)OCc1ccc(NC(=O)[C@H](CC(=O)OC(C)(C)C)NC(=O)[C@H](CCCCNC(=O)OC(C)(C)C)NC(=O)CCOCCOCCOCCN=[N+]=[N-])cc1)CC1CCCC1C2=O]

To a solution of 4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl 8-hydroxy-7-methoxy-5-oxo-2,3,11,11a-tetrahydro-1H-benzo[e]pyrrolo[1,2-a][1,4]diazepine-10(5H)-carboxylate (2.02 g, 1.94 mmol) in butanone (50 ml) was added Cs₂CO₃ (2.50 g, 7.67 mmol) and 1,3-diiodopropane (2.50 g, 8.45 mmol). The mixture was stirred at 45° C. under dark for 36 h, concentrated and purified on SiO₂ column eluted with EtOAc/CH₂Cl₂ (1:5) to afford the title compound (2.08 g, 90% yield). MS ESI m/z calcd for C₅₂H₇₇IN₉O₁₅ [M+H]⁺ 1194.45, found 1194.95.

### Example 136. Synthesis of (S)-2-((S)-1-azido-14-methyl-12-oxo-3,6,9-trioxa-13-azapentadecanamido)-N-(4-(hydroxymethyl)phenyl)propanamide

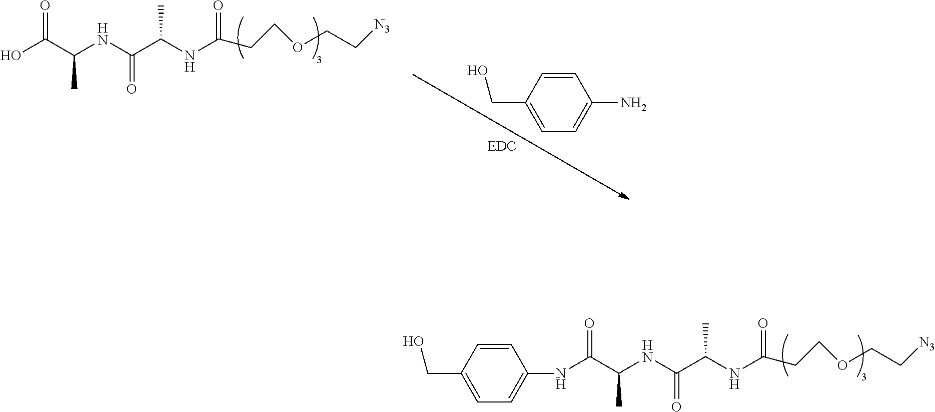

To a solution of (14S,17S)-1-azido-14,17-dimethyl-12,15-dioxo-3,6,9-trioxa-13,16-diazaoctadecan-18-oic acid (3.02 g, 7.75 mmol) and (4-aminophenyl)methanol (1.05 g, 8.53 mmol) in DMA was added EDC (4.90 g, 25.52 mmol). The mixture was stirred at RT for 14 h, concentrated and purified on SiO₂ column eluted with EtOAc/CH₂Cl₂ (1:8 to 1:3) to afford the title compound (3.52 g, 92% yield). MS ESI m/z calcd for C₂₂H₃₅IN₆O₇ [M+H]⁺ 495.25, found 495.60.

### Example 137. Synthesis of (11R,11aS)-4-((14S,17S)-1-azido-14,17-dimethyl-12,15-dioxo-3,6,9-trioxa-13,16-diazaoctadecanamido)benzyl 8-(benzyloxy)-11-hydroxy-7-methoxy-2-methylene-5-oxo-2,3,11,11a-tetrahydro-1H-benzo[e]pyrrolo[1,2-a][1,4]diazepine-10(5H)-carboxylate

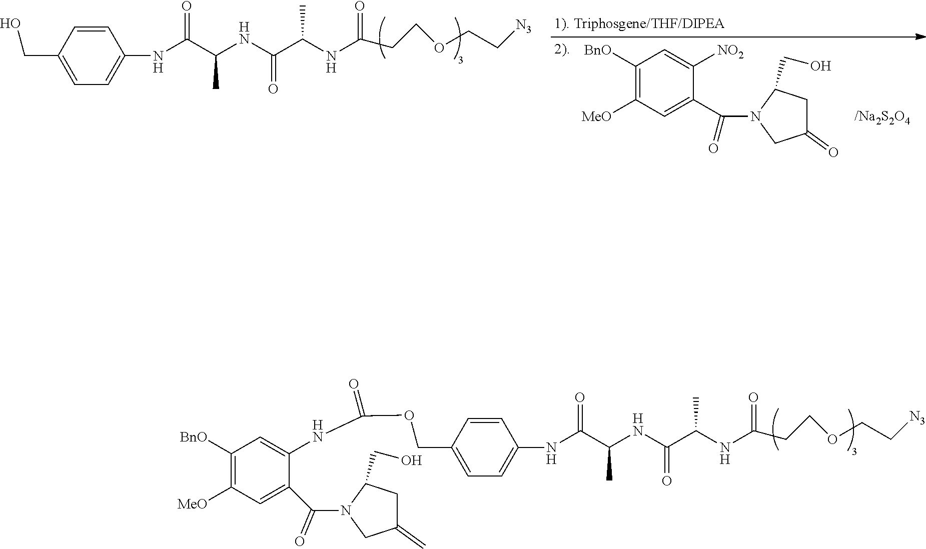

A mixture of (S)-(4-(benzyloxy)-5-methoxy-2-nitrophenyl)(2-(hydroxymethyl)-4-methylene-pyrrolidin-1-yl)methanone (3.90 g, 9.80 mmol) and Na₂S₂O₄ (6.0 g, 34.47 mmol) in THF (60 ml) and H₂O (40 ml) was stirred at room temperature for 20 h, adjusted pH to 10 with Na₂CO₃, concentrated, purified on C-18 short column eluted with H₂O/MeOH/Et₃N (from 99.4/0.5/0.2 to 50/49.8/0.2). The fractions containing the reduced amino product were pooled, concentrated, diluted with THF (50 ml), then cooled to 4-8° C. Separately to a solution of 2-(1-azido-14-methyl-12-oxo-3,6,9-trioxa-13-azapentadecanamido)-N-(4-(hydroxy-methyl)phenyl)-propanamide (6.70 g, 13.56 mmol) in dry THE (150 mL) was added DIPEA (3.50 g, 27.12 mmol) and a solution of triphosgene (4.10 g, 13.80 mmol) in dry THE (20 mL) at 4-8° C. After 15 min of stirring at 4-8° C., the solution was added dropwise to the above amino solution at 4-8° C. for 45 min. The mixture was warmed to RT and continued to stir for 2 h, concentrated, extracted with CH₂Cl₂ (3×30 ml), dried over Na₂SO₄, evaporated and and purified on SiO₂ column eluted with EtOAc/CH₂Cl₂ (1:10 to 1:5) to afford the title compound (7.23 g, 83% yield in two steps). MS ESI m/z calcd for C₄₅H₅₇IN₈O₁₂ [M+H]⁺ 889.40, found 889.90.

### Example 138. Synthesis of (11S,11aS)-4-((14S,17S)-1-azido-14,17-dimethyl-12,15-dioxo-3,6,9-trioxa-13,16-diazaoctadecanamido)benzyl 8-(benzyloxy)-11-hydroxy-7-methoxy-2-methylene-5-oxo-2,3,11,11a-tetrahydro-1H-benzo[e]pyrrolo[1,2-a][1,4]diazepine-10(5H)-carboxylate

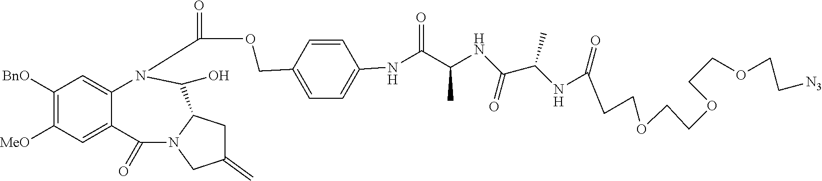

To a solution of (11R,11aS)-4-((14S,17S)-1-azido-14,17-dimethyl-12,15-dioxo-3,6,9-trioxa-13,16-diazaoctadecanamido)benzyl 8-(benzyloxy)-11-hydroxy-7-methoxy-2-methylene-5-oxo-2,3,11,11a-tetrahydro-1H-benzo[e]pyrrolo[1,2-a][1,4]diazepine-10(5H)-carboxylate (3.80 g, 4.27 mmol) in dry DCM (40 mL) was added Dess-Martin periodinane (DMP) (2.80 g, 6.60 mmol) under nitrogen at room temperature. After completion of conversion, the reaction solution was added aqueous Na₂SO₃ and followed by aqueous NaHCO₃, the mixture was stirred for further 15 minutes and extracted with DCM (3×20 mL). The combined organic extract was washed with brine, dried, filtered and concentrated. The residue was purified by SiO₂ chromatography (DCM/EtOAc=5/1 to 2:1) to give the title compound (2.99 g, 79% yield) as an off-white foam. MS ESI m/z calcd for C₄₄H₅₅N₈O₁₂ [M+H]⁺ 886.39, found 886.80.

### Example 139. Synthesis of (11S,11aS)-4-((14S,17S)-1-azido-14,17-dimethyl-12,15-dioxo-3,6,9-trioxa-13,16-diazaoctadecanamido)benzyl 8,11-dihydroxy-7-methoxy-2-methylene-5-oxo-2,3,11,11a-tetrahydro-1H-benzo[e]pyrrolo[1,2-a][1,4]diazepine-10(5H)-carboxylate

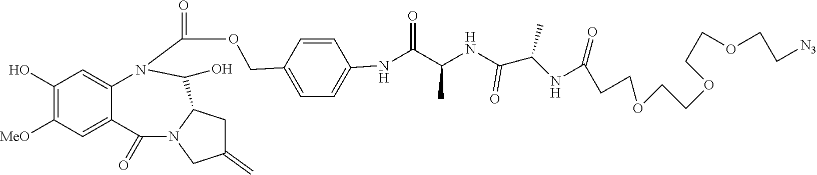

To a solution of (11S,11aS)-4-((14S,17S)-1-azido-14,17-dimethyl-12,15-dioxo-3,6,9-trioxa-13,16-diazaoctadecanamido)benzyl 8-(benzyloxy)-11-hydroxy-7-methoxy-2-methylene-5-oxo-2,3,11,11a-tetrahydro-1H-benzo[e]pyrrolo[1,2-a][1,4]diazepine-10(5H)-carboxylate (2.90 g, 3.27 mmol) in 40 ml of CH₂Cl₂ was added 15 ml of CH₃SO₃H at 0° C. The mixture was stirred at 0° C. for 10 min then r.t. for 1 h, diluted with CH₂Cl₂, pH adjusted with cold 1.0 N NaHCO₃ to 4 and filtered. The aqueous layer was extracted with CH₂Cl₂(3×60 ml). The organic layers were combined, dried over Na₂SO₄, filtered, evaporated and purified on SiO₂ column chromatography (CH₃OH/CH₂Cl₂ 1:15 to 1:5) to afford 1.95 g (75% yield) of the title product. MS ESI m/z calcd for C₃₇H₄₈IN₈O₁₂ [M+H]⁺ 797.34, found 797.90.

### Example 140. Synthesis of (11S,11aS)-4-((14S,17S)-1-azido-14,17-dimethyl-12,15-dioxo-3,6,9-trioxa-13,16-diazaoctadecanamido)benzyl 8-(3-(((S)-10-(((4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate

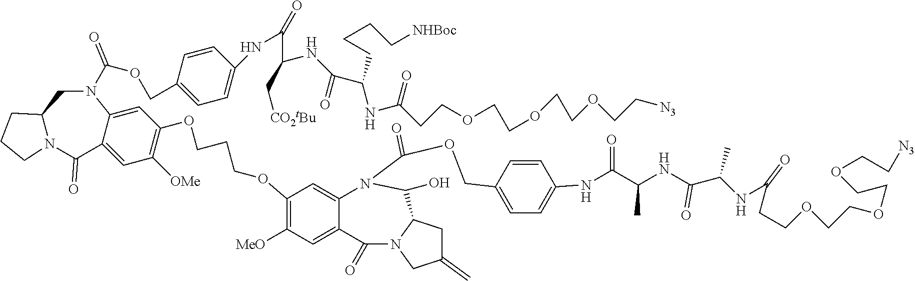

To a solution of (11S,11aS)-4-((14S,17S)-1-azido-14,17-dimethyl-12,15-dioxo-3,6,9-trioxa-13,16-diazaoctadecanamido)benzyl 8,11-dihydroxy-7-methoxy-2-methylene-5-oxo-2,3,11,11a-tetrahydro-1H-benzo[e]pyrrolo[1,2-a][1,4]diazepine-10(5H)-carboxylate (402 mg, 0.504 mmol) and (S)-4-((14S,17S)-1-azido-17-(2-(tert-butoxy)-2-oxoethyl)-14-(4-((tert-butoxycarbonyl)amino)-butyl)-12,15-dioxo-3,6,9-trioxa-13,16-diazaoctadecanamido)benzyl 8-(3-iodopropoxy)-7-methoxy-5-oxo-2,3,11,11a-tetrahydro-1H-benzo[e]pyrrolo[1,2-a][1,4]diazepine-10(5H)-carboxylate (650 mg, 0.544 mmol) in butanone (50 ml) was added Cs₂CO₃ (0.50 g, 1.53 mmol). The mixture was stirred at 45° C. under dark for 36 h, concentrated and purified on SiO₂ column eluted with EtOAc/CH₂Cl₂ (1:8 to 1:3) to afford the title compound (809 mg, 86% yield). MS ESI m/z calcd for C₈₉H₂₄N₁₇O₂₇ [M+H]⁺ 1862.89, found 1863.45.

### Example 141. Synthesis of (11S,11aS)-4-((14S,17S)-1-amino-14,17-dimethyl-12,15-dioxo-3,6,9-trioxa-13,16-diazaoctadecanamido)benzyl 8-(3-(((S)-10-(((4-((14S,17S)-1-amino-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate

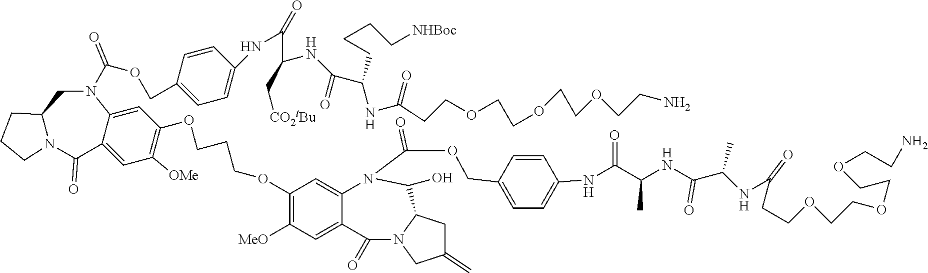

(11S,11aS)-4-((14S,17S)-1-Azido-14,17-dimethyl-12,15-dioxo-3,6,9-trioxa-13,16-diazaoctadecanamido)benzyl 8-(3-(((S)-10-(((4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)-benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate (750 mg, 0.402 mmol) in THE (8 ml) was added Me₃P (1.0 M in toluene, 2.0 ml, 2.0 mmol) at 0-4° C. under N2. After stirred for 5 min, the ice bath was removed and the reaction mixture was stirred at RT for 2 h. Then, water (1 ml) was added and the mixture was stirred for 10 min. The mixture was diluted with 1,4-dioxane (10 ml), concentrated and co-evaporated with dioxane/toluene to dryness to yield the crude amino product (725 mg, ˜99% yield) which was used directly for next step without further purification. MS ESI m/z calcd for C₈₉H₂₈N₁₃O₂₇ [M+H]⁺ 1810.90, found 1811.50.

### Example 142. Synthesis of an Asymmetrically Cross-Linked PBD Dimer C-9

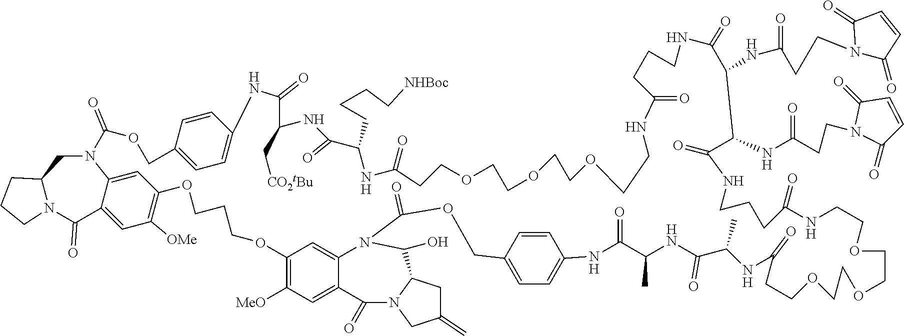

To the above crude amino compound ((11S,11aS)-4-((14S,17S)-1-amino-14,17-dimethyl-12,15-dioxo-3,6,9-trioxa-13,16-diazaoctadecanamido)benzyl 8-(3-(((S)-10-(((4-((14S,17S)-1-amino-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate) in dry DMA (8 ml) was added 4,4′-(((2R,3S)-2,3-bis(3-(2,5-dioxo-2,5-dihydro-1H-pyrrol-1-yl)propanamido)succinyl)bis(azanediyl))dibutanoic acid (248.0 mg, 0.400 mmol) and EDC (500.0 mg, 2.60 mmol). The mixture was a stirred for 24 h, concentrated and purified on C₁₈ preparative HPLC (17% C₁₈, 250 mm×50 mm), eluted with water/CH₃CN (from 80% water to 30% water in 40 min, 9 ml/min) to afford 488.1 mg (51% yield) of the C-9 product after drying under high vacuum. ESI MS m/z C₁₁₅H₁₅₆N₁₉O₃₇ [M+H]⁺, cacld. 2395.08, found 2395.90.

### Example 143. Synthesis of an Unsymmetrically Cross-Linked PBD Dimer C-10

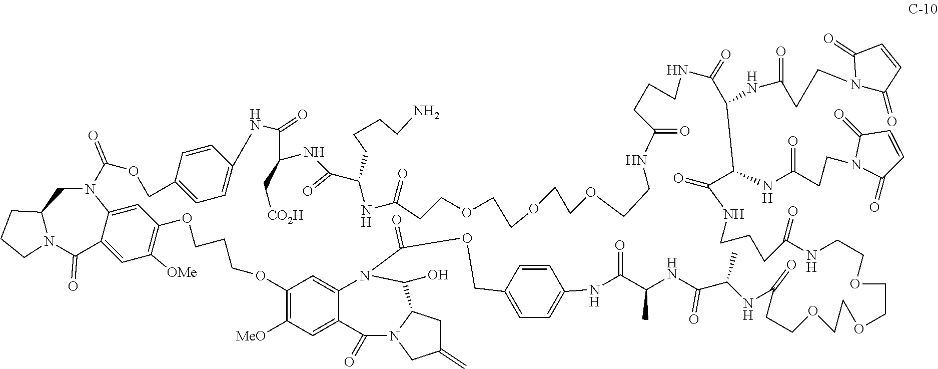

C-9 compound (465.0 mg, 0.194 mmol) was dissolved in DCM (4 ml), followed by addition of TFA (2 ml) at 0-4° C. The reaction mixture was then stirred at RT for 1 h, diluted with toluene (5 ml), then concentrated, and co-evaporated with DCM/toluene to dryness to afford the crude product C-3 (48.0 mg, 100% yield, 92% pure by HPLC) which was further purified by reverse phase HPLC (250 (L) mm x 20(d) mm, C₁₈ column, 5-60% acetonitrile/water in 40 min, v=8 ml/min) to afford the pure product C-10 (373.1 mg, 85% yield, 96% pure) as a foam. ESI MS m/z: calcd for C₁₀₆H₁₄₀N₁₉O₃₅ [M+H]+ 2238.97, found 2239.50.

### Example 144. Synthesis of an Asymmetrically Cross-Linked PBD Dimer C-11

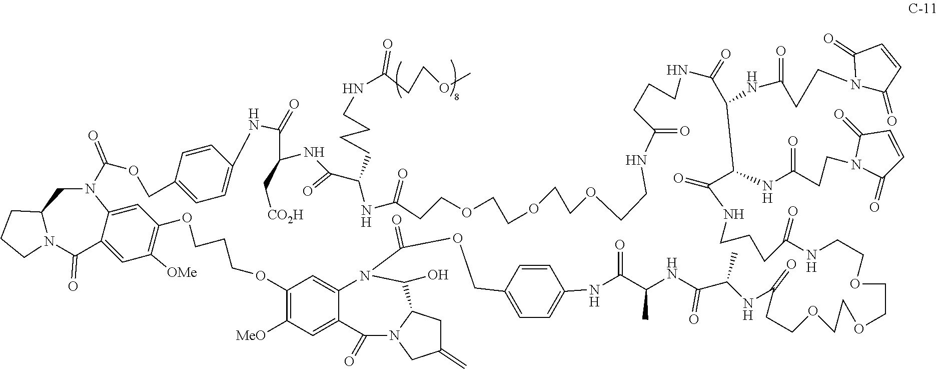

C-10 compound (235.0 mg, 0.105 mmol) was dissolved in a mixture solution of THF (3 ml) and 0.1 M, NaH₂PO₄ (3 ml), pH 7.5, followed by addition of N-succinimidyl 2,5,8,11,14,17,20,23-octaoxahexacosan-26-oate (43.0 mg, 0.084 mmol) in 4 portions in 2 h. The reaction mixture was then continued to stir at RT for 4 h, and co-evaporated with DMF (10 ml) to dryness to afford the crude product C-11 which was further purified by reverse phase HPLC (250 (L) mm x 50(d) mm, C₁₈ column, 20-60% acetonitrile/water in 40 min, v=8 ml/min) to afford the pure product C-11 (215.5 mg, 78% yield, 95% pure) as a foam. ESI MS m/z: calcd for C₁₂₄H₁₇₄N₁₉O₄₄ [M+H]⁺ 2633.20, found 2633.85.

### Example 145. Synthesis of an Asymmetrically Cross-Linked PBD Dimer C-12

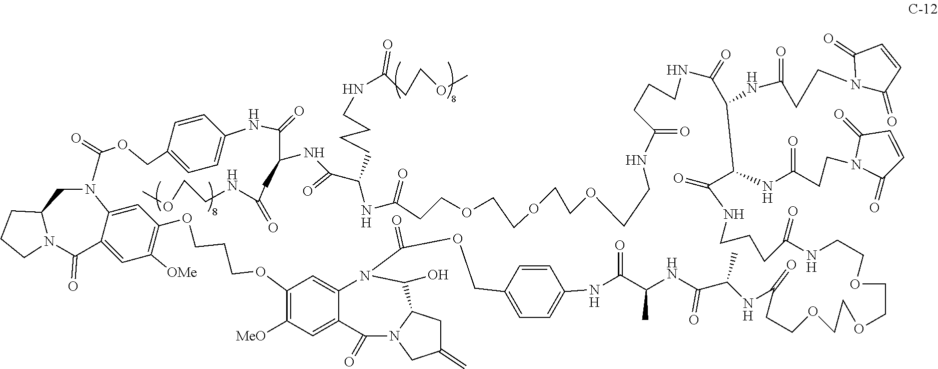

To a solution of C-11 compound (65.0 mg, 0.0246 mmol) and 2,5,8,11,14,17,20,23-octaoxapentacosan-25-amine (15.1 mg, 0.0394 mmol) in dry DMA (2 ml) was added EDC (30.0 mg, 0.156 mmol). The reaction mixture was stirred at RT for 15 h, concentrated, purified by reverse phase HPLC (250 (L) mm x 30 (d) mm, C₁₈ column, 20-60% acetonitrile/water in 40 min, v=8 ml/min) to afford the pure product C-12 (60.2 mg, 81% yield, 95% pure by HPLC) as a foam. ESI MS m/z: calcd for C₁₄₁H₂₀₉N₂₀O₅₁ [M+H]+ 2998.43, found 2999.40.

### Example 146. Synthesis of an Asymmetrically Cross-Linked PBD Dimer C-13

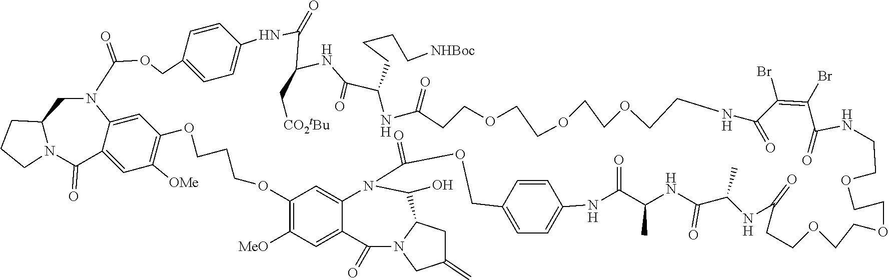

To the crude amino compound ((11S,11aS)-4-((14S,17S)-1-amino-14,17-dimethyl-12,15-dioxo-3,6,9-trioxa-13,16-diazaoctadecanamido)benzyl 8-(3-(((S)-10-(((4-((14S,17S)-1-amino-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate) (˜120 mg, ˜0.0662 mmol) in dry THE (10 ml) was added 3,4-dibromofuran-2,5-dione (16.8 mg, 0.0661 mmol). The mixture was stirred at RT for 4 h, then EDC (50.2 mg, 0.261 mmol). The mixture was continuously stirred for 12 h, concentrated, purified by SiO₂ column, eluted with MeOH/CH₂Cl₂ (1:12 to 1:6) to afford the pure product C-13 (112.2 mg, 83% yield) as a foam. ESI MS m/z: calcd for C₉₃H₁₂₆Br₂N₁₃O₂₉ [M+H]⁺ 2046.7073, found 2046.8260.

### Example 147. Synthesis of an Asymmetrically Cross-Linked PBD Dimer C-14

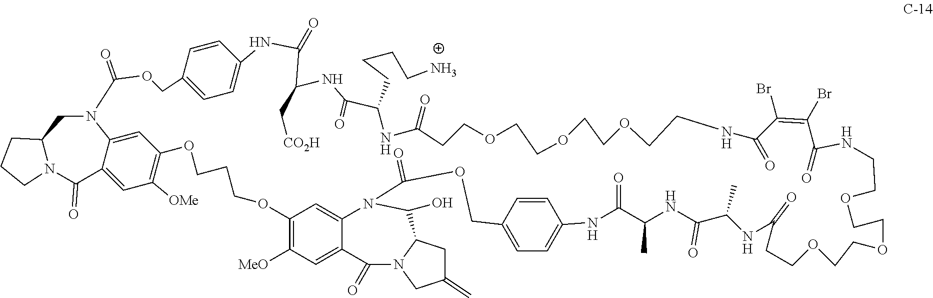

C-13 compound (100.2 mg, 0.0489 mmol) was dissolved in DCM (4 ml), followed by addition of TFA (2 ml) at 0-4° C. The reaction mixture was then stirred at RT for 1 h, diluted with toluene (5 ml), then concentrated, and co-evaporated with DCM/toluene to dryness to afford the crude product C-14 (94.3 mg, 102% yield, 93% pure by HPLC) which was further purified by reverse phase HPLC (250 (L) mm x 20(d) mm, C₁₈ column, 5-60% acetonitrile/water in 40 min, v=8 ml/min) to afford the pure product C-14 (76.6 mg, 83% yield, 96% pure) as a foam. ESI MS m/z: calcd for C₈₄H₁₀₉Br₂N₁₃O₂₇ [M+H]⁺ 1890.5995, found 1890.6250, 1893.6565 [M+H+2]⁺.

### Example 148. Synthesis of an Asymmetrically Cross-Linked PBD Dimer C-15

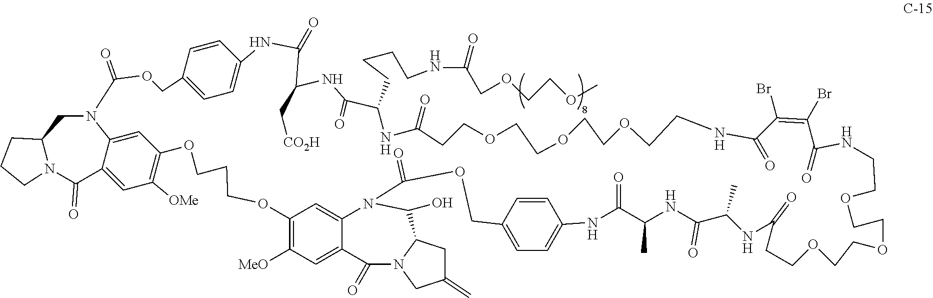

C-14 compound (55.0 mg, 0.0291 mmol) was dissolved in a mixture solution of THF (3 ml) and 0.1 M, NaH₂PO₄ (3 ml), pH 7.5, followed by addition of N-succinimidyl 2,5,8,11,14,17,20,23,26-nonaoxaoctacosan-28-oate (47.2 mg, 0.0875 mmol) in 4 portions in 2 h. The reaction mixture was then continued to stir at RT for 4 h, and co-evaporated with DMF (10 ml) to dryness to afford the crude product C-15 which was further purified by reverse phase HPLC (250 (L) mm x 50(d) mm, C₁₈ column, 20-60% acetonitrile/water in 40 min, v=8 ml/min) to afford the pure product C-15 (215.5 mg, 78% yield, 95% pure) as a foam. ESI MS m/z: calcd for C₁₀₃H₁₄₆Br₂N₁₃O₃₇ [M+H]⁺ 2314.8309, found 2314.8575, 2316.8705 [M+H+2]⁺, 2318.1445 [M+H+4]⁺.

### Example 149. Synthesis of an Asymmetrically Cross-Linked PBD Dimer C-16

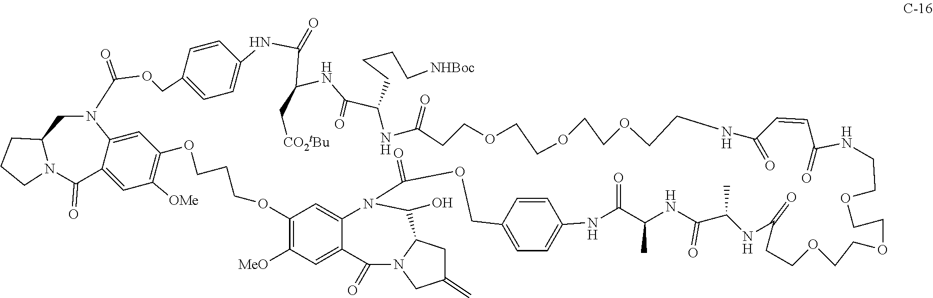

To the crude amino compound ((11S,11aS)-4-((14S,17S)-1-amino-14,17-dimethyl-12,15-dioxo-3,6,9-trioxa-13,16-diazaoctadecanamido)benzyl 8-(3-(((S)-10-(((4-((14S,17S)-1-amino-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate) (˜120 mg, ˜0.0662 mmol) in dry THE (10 ml) was added 3,4-furan-2,5-dione (6.5 mg, 0.0661 mmol). The mixture was stirred at RT for 4 h, then EDC (50.2 mg, 0.261 mmol). The mixture was continuously stirred for 12 h, concentrated, purified by SiO₂ column, eluted with MeOH/CH₂Cl₂ (1:12 to 1:6) to afford the pure product C-16 (107.3 mg, 86% yield) as a foam. ESI MS m/z: calcd for C₉₃H₂₈N₁₃O₂₉ [M+H]⁺ 1890.8941, found 1890.8990.

### Example 150. Synthesis of an Asymmetrically Cross-Linked PBD Dimer C-17

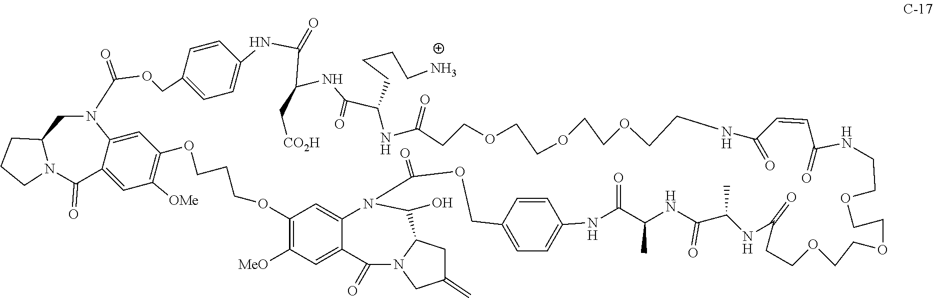

C-16 compound (85.5 mg, 0.0452 mmol) was dissolved in DCM (4 ml), followed by addition of TFA (2 ml) at 0-4° C. The reaction mixture was then stirred at RT for 1 h, diluted with toluene (5 ml), then concentrated, and co-evaporated with DCM/toluene to dryness to afford the crude product C-17 (81.3 mg, 104% yield, 92% pure by HPLC) which was further purified by reverse phase HPLC (250 (L) mm x 20(d) mm, C₁₈ column, 5-60% acetonitrile/water in 40 min, v=8 ml/min) to afford the pure product C-17 (67.4 mg, 86% yield, 96% pure) as a foam. ESI MS m/z: calcd for C₈₄H₁₁₂N₁₃O₂₇ [M+H]⁺ 1734.7785, found 1734.8285.

### Example 151. Synthesis of an Asymmetrically Cross-Linked PBD Dimer C-18

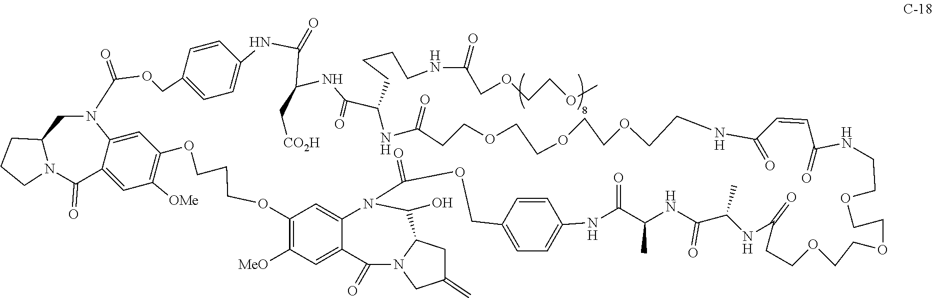

C-17 compound (53.0 mg, 0.0305 mmol) was dissolved in a mixture solution of THF (3 ml) and 0.1 M, NaH₂PO₄ (3 ml), pH 7.5, followed by addition of N-succinimidyl 2,5,8,11,14,17,20,23,26-nonaoxaoctacosan-28-oate (47.0 mg, 0.0874 mmol) in 4 portions in 2 h. The reaction mixture was then continued to stir at RT for 4 h, and co-evaporated with DMF (10 ml) to dryness to afford the crude product C-18 which was further purified by reverse phase HPLC (250 (L) mm x 50(d) mm, C₁₈ column, 20-60% acetonitrile/water in 40 min, v=8 ml/min) to afford the pure product C-18 (53.25 mg, 83% yield, 95% pure) as a foam. ESI MS m/z: calcd for C₁₀₃H₁₄₈N₁₃O₃₇ [M+H]⁺ 2159.0099, found 2159.0890.

### Example 152. Synthesis of an Asymmetrically Cross-Linked PBD Dimer C-19

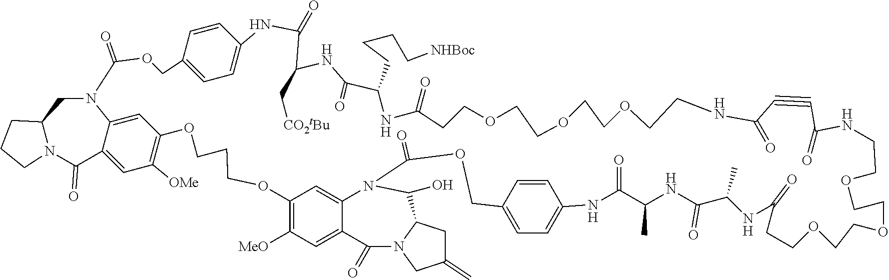

To the crude amino compound ((11S,11aS)-4-((14S,17S)-1-amino-14,17-dimethyl-12,15-dioxo-3,6,9-trioxa-13,16-diazaoctadecanamido)benzyl 8-(3-(((S)-10-(((4-((14S,17S)-1-amino-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate) (˜120 mg, ˜0.0662 mmol) in dry THE (10 ml) was added but-2-ynedioic acid (7.5 mg, 0.0661 mmol) and EDC (50.2 mg, 0.261 mmol). The mixture was stirred at RT for 12 h, concentrated, purified by SiO₂ column, eluted with MeOH/CH₂Cl₂ (1:12 to 1:6) to afford the pure product C-19 (86.3 mg, 69% yield) as a foam. ESI MS m/z: calcd for C₉₃H₂₆N₁₃O₂₉ [M+H]+ 1888.8784, found 1888.8895.

### Example 153. Synthesis of an Asymmetrically Cross-Linked PBD Dimer C-20

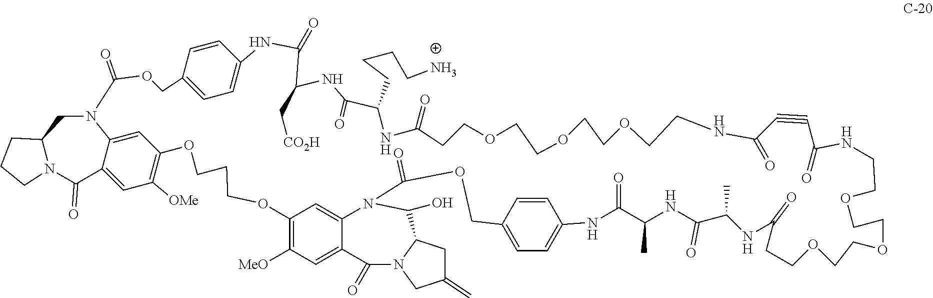

C-19 compound (75.5 mg, 0.0397 mmol) was dissolved in DCM (4 ml), followed by addition of TFA (2 ml) at 0-4° C. The reaction mixture was then stirred at RT for 1 h, diluted with toluene (5 ml), then concentrated, and co-evaporated with DCM/toluene to dryness to afford the crude product C-17 (72.2 mg, 105% yield, 91% pure by HPLC) which was further purified by reverse phase HPLC (250 (L) mm x 20(d) mm, C₁₈ column, 5-60% acetonitrile/water in 40 min, v=8 ml/min) to afford the pure product C-20 (55.7 mg, 81% yield, 95% pure) as a foam. ESI MS m/z: calcd for C₈₄H₁₁₀N₁₃O₂₇ [M+H]+ 1732.7629, found 1732.8025.

### Example 154. Synthesis of an Asymmetrically Cross-Linked PBD Dimer C-21

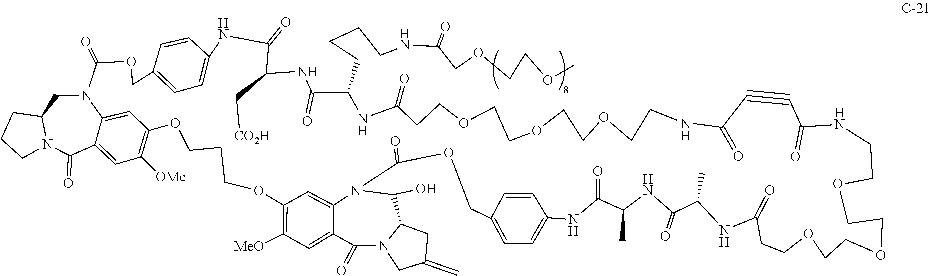

C-20 compound (45.0 mg, 0.026 mmol) was dissolved in a mixture solution of THF (3 ml) and 0.1 M, NaH₂PO₄ (3 ml), pH 7.5, followed by addition of N-succinimidyl 2,5,8,11,14,17,20,23,26-nonaoxaoctacosan-28-oate (47.0 mg, 0.0874 mmol) in 4 portions in 2 h. The reaction mixture was then continued to stir at RT for 4 h, and co-evaporated with DMF (10 ml) to dryness to afford the crude product C-18 which was further purified by reverse phase HPLC (250 (L) mm×50(d) mm, C₁₈ column, 206% acetonitrile/water in 40 min v=8 ml/min) to afford the pure product C-21 (45.3 mg, 81% yield, 95 pure) as a foam. ESI MS m/z.: calcd for C₁₀₃H₁₄₆N₁₃O₃₇ [M+H]⁺ 2156.9943, found 2157.1250.

### Example 155. Synthesis of a Cross-Linked PBD Dimer C-22

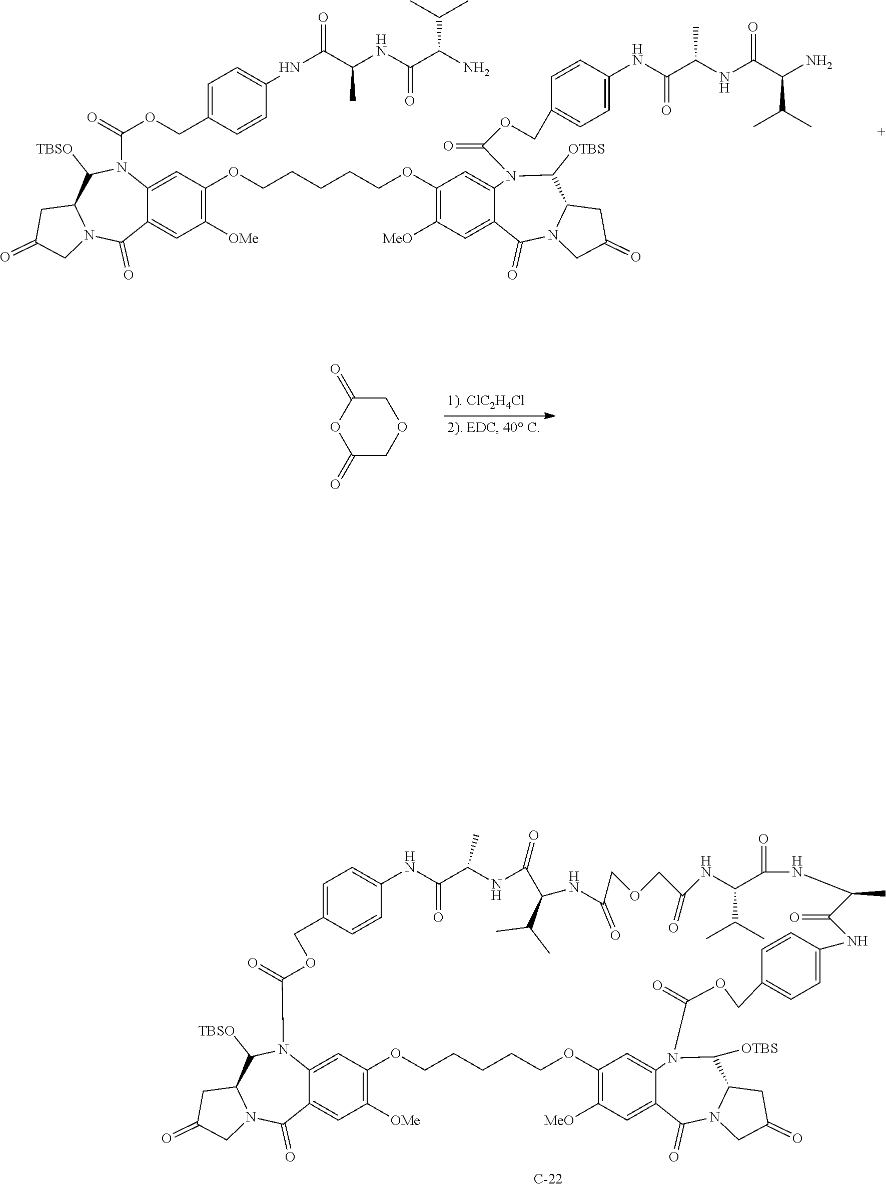

(11S,11aS,11′S,11a′S)-bis(4-((S)-2-((S)-2-amino-3-methylbutanamido)propanamido)benzyl) 8,8′-(pentane-1,5-diylbis(oxy))bis(11-((tert-butyldimethylsilyl)oxy)-7-methoxy-2,5-dioxo-2,3,11,11a-tetrahydro-1H-benzo[e]pyrrolo[1,2-a][1,4]diazepine-10(5H)-carboxylate) (2.26 g, 1.51 mmol) in dichloroethane (40 ml) was added 1,4-dioxane-2,6-dione (176 mg, 1.51 mmol). The mixture was stirred at RT for 4 h, then EDC (1.16 g, 6.04 mmol) and DIPEA (0.40 g, 3.10 mmol) were added in. The mixture was stirred at 40° C. for 24 h, evaporated and purified on SiO₂ column chromatography (CH₃OH/CH₂Cl₂ 1:15 to 1:5) to afford 1.99 g (83% yield) of the C-22 compound. MS ESI m/z calcd for C₇₉H₁₀₉N₁₀O₂₁Si₂ [M+H]⁺ 1589.7307, found 1589.9025.

### Example 156. Synthesis of a Cross-Linked PBD Dimer C-23

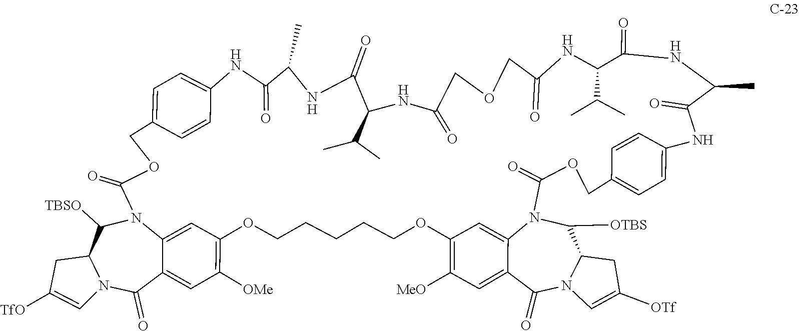

Compound C-22 (1.98 g, 1.24 mmol) in the mixture of anhydrous CH₂Cl₂ (30 ml) and 2,6-lutidine (2.0 ml, 17.16 mmol) at −45° C. under N2 was triflic anhydride (2.68 ml, 15.93 mmol). The mixture was stirred at −45° C. for 2 h, diluted with CH₂Cl₂ (30 ml), washed with water (50 ml), 5% acetic acid (2×80 ml), saturated NaHCO₃(2×80 ml), brine (80 ml) and dried over Na₂SO₄. Filtration and evaporation of the solvent in vacuo afford the crude product which was purified by SiO₂ column eluted with EtOAc/CH₂Cl₂ (1:10 to 1:6) to afford C-23 as a white foam (1.68 g, 74% yield). MS ESI m/z calcd for C₈₁H₁₀₇F₆N₁₀O₂₅S₂Si₂ [M+H]⁺ 1583.6293, found 1583.7055.

### Example 157. Synthesis of a Cross-Linked PBD Dimer C-24

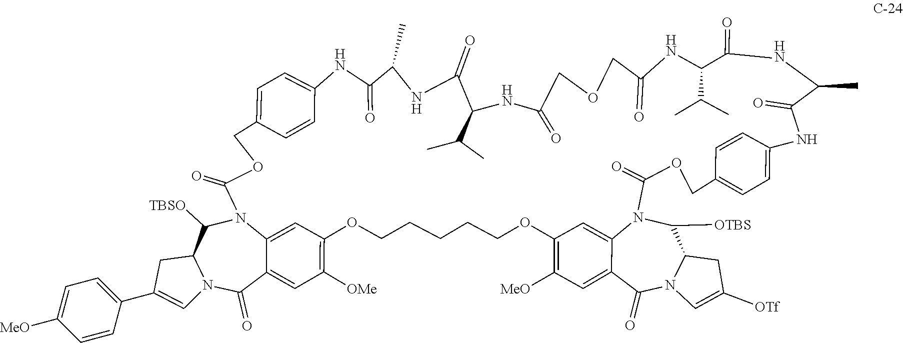

To the solution of C-23 (348.1 mg, 0.22 mmol) in the mixture of toluene (3 mL), EtOH (10 mL), in H₂O (1.5 mL) at room temperature were added solid Pd(PPh₃)₄ (10 mg, 8.69 .mmol), 4-methoxyphenyl boronic acid (40 mg, 0.26 mmol), Na₂CO₃ (37 mg, 0.35 mmol). The reaction mixture was allowed to stir under N2 for 24 h, at which point the reaction was deemed complete as judged by LC/MS and TLC (EtOAc). The solvent was removed in vacuo and the resulting residue partitioned between EtOAc (100 mL) and H₂O (100 mL). The aqueous phase was extracted with EtOAc (3×40 mL) and the combined organic layers washed with H₂O (40 mL), brine (40 mL), dried over Na₂SO₄, filtered and evaporated to provide the crude product which was purified by SiO₂ flash chromatography (eluted with EtOAc/CH₂Cl₂, 1:10 to 1:6) to provide compound C-24 as a white foam (286 mg, 72% yield). MS ESI m/z calcd for C₈₇H₁₁₄F₃N₁₀O₂₃Si₂ [M+H]⁺ 1811.7270, found 1811.7965.

### Example 158. Synthesis of a Cross-Linked PBD Dimer C-25

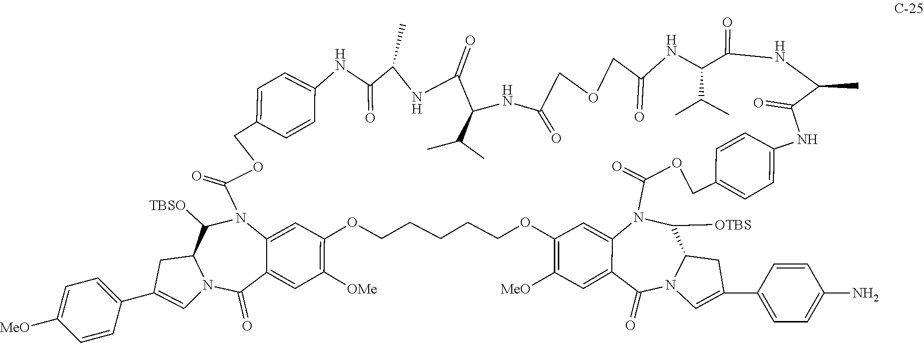

To the solution of C-24 (250.1 mg, 0.138 mmol) in the mixture of toluene (3 mL), EtOH (10 mL), in H₂O (1.5 mL) at room temperature were added solid Pd(PPh₃)₄ (10 mg, 8.69 .mmol), 4-(4,4,5,5-tetramethyl-1,3,2-dioxaboralane-2-yl)aniline (60 mg, 0.27 mmol), Na₂CO₃ (40 mg, 0.37 mmol). The reaction mixture was allowed to stir under N₂ for 24 h, at which point the reaction was deemed complete as judged by LC/MS and TLC (EtOAc). The solvent was removed in vacuo and the resulting residue partitioned between EtOAc (100 mL) and H₂O (100 mL). The aqueous phase was extracted with EtOAc (3×40 mL) and the combined organic layers washed with H₂O (40 mL), brine (40 mL), dried over Na₂SO₄, filtered and evaporated to provide the crude product which was purified by SiO₂ flash chromatography (eluted with EtOH/CH₂Cl₂, 1:15 to 1:8) to provide compound C-25 as a pale foam (142 mg, 59% yield). MS ESI m/z calcd for C₉₂H₁₂₀N₁₁O₂₀Si₂ [M+H]⁺ 1754.8250, found 1754.9830.

### Example 159. Synthesis of Tert-butyl 2,5,8,11,14,17,20,23,26,29-decaoxahentriacontan-31-oate

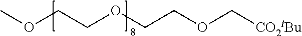

NaH (60%, 8.0 g, 200 mmol) was added to a solution of 2,5,8,11,14,17,20,23,26-nonaoxaoctacosan-28-ol (42.8 g, 100 mmol) in THF (1.0 L). After stirring at r.t. for 30 min, tert-butyl 2-bromoacetate (48.8 g, 250 mmol) was added to the mixture, and stirred at r.t. for 1 h. The mixture was then poured onto ice water, extracted with DCM, and the organic layer was washed with brine, dried over anhydrous Na₂SO₄. Purification by column chromatography (0% to 5% MeOH: DCM) yielded the title compound as a yellow oil (32 g, 59% yield). ESI MS 543.35 [M+H]⁺.

### Example 160. Synthesis of 2,5,8,11,14,17,20,23,26,29-decaoxahentriacontan-31-oic Acid

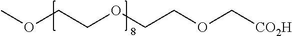

Tert-butyl 2,5,8,11,14,17,20,23,26,29-decaoxahentriacontan-31-oate (40.0 g, 73.8 mmol) was dissolved in DCM (400 mL), and then formic acid (600 mL) was added. The resulting solution was stirred at 25° C. overnight. All volatiles were removed under vacuum, which afforded the title product as a yellow oil (36.0 g, ˜100% yield). ESI m/z calcd for C₂₁H₄₃O₁₂ [M+H]⁺: 487.27, found 487.24.

### Example 161. Synthesis of 2,5,8,11,14,17,20,23,26,29-decaoxahentriacontan-31-oyl Chloride

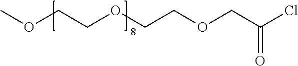

To the solution of 2,5,8,11,14,17,20,23,26,29-decaoxahentriacontan-31-oic acid (36.0 g, 73.8 mmol) dissolved in DCM (640 mL), (COCl)₂ (100 mL) and DMF (52 g, 0.74 mmol) were added. The resulting solution was stirred at r.t. for 4 h. All volatiles were removed under vacuum to yield the title product as a yellow oil.

### Example 162. Synthesis of (S)-37-(((benzyloxy)carbonyl)amino)-31-oxo-2,5,8,11,14,17,20,23,26,29-decaoxa-32-azaoctatriacontan-38-oic Acid

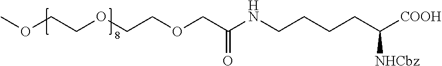

Z-L-Lys-OH (41.4 g, 147.6 mmol), Na₂CO₃ (23.4 g, 221.4 mmol) and NaOH (5.9 g, 147.6 mmol) were dissolved in water (720 mL). The mixture was cooled to 0° C., to which a solution of of 2,5,8,11,14,17,20,23,26,29-decaoxahentriacontan-31-oyl chloride. (37.2 g, 73.8 mmol) in THE (20 mL) was added. The resulting mixture was stirred at r.t. for 1 h. THE was removed under vacuum, and concentrated HCl was added to the aqueous solution until pH reached 3 under ice cooling. After extraction with DCM, the organic layer was washed with brine, dried over Na₂SO₄ and concentrated to give the title product as a yellow oil (55 g, 99% yield). ESI m/z calcd for C₃₅H₆₀N₂O₁₅ [M+H]⁺: 749.40, found 749.39.

### Example 163. Synthesis of (S)-tert-butyl 37-(((benzyloxy)carbonyl)amino)-31,38-dioxo-2,5,8,11,14,17,20,23,26,29-decaoxa-32,39-diazatritetracontan-43-oate

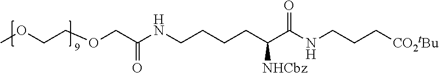

A mixture of tert-butyl 4-aminobutanoate (1.03 g, 6.12 mmol) and (S)-37-(((benzyloxy)-carbonyl)amino)-31-oxo-2,5,8,11,14,17,20,23,26,29-decaoxa-32-azaoctatriacontan-38-oic acid (4.16 g, 5.56 mmol) in DMF (18 mL) was cooled to 0° C. and HATU (2.32 g, 6.12 mmol) and TEA (1.2 mL, 8.34 mmol) were added in sequence. The reaction was stirred for 50 min, then diluted with water (300 mL), and extracted with EtOAc (3×250 mL). The EtOAc solution was washed with brine, dried over anhydrous Na₂SO₄, filtered, concentrated and purified by SiO₂ column chromatography (32:1 DCM/MeOH) to give the title compound (4.40 g, 89% yield). MS ESI m/z calcd for C₄₃H₇₅N₃O₁₆ [M+H]⁺ 890.51, found 891.09.

### Example 164. Synthesis of (S)-tert-butyl 37-amino-31,38-dioxo-2,5,8,11,14,17,20,23,26,29-decaoxa-32,39-diazatritetracontan-43-oate

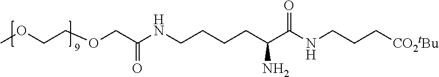

To a solution of (S)-tert-butyl 37-(((benzyloxy)carbonyl)amino)-31,38-dioxo-2,5,8,11,14,17,20,23,26,29-decaoxa-32,39-diazatritetracontan-43-oate (1 g, 1.13 mmol) in MeOH (50 mL) was added Pd/C (10 wt %, 0.10 g) in a hydrogenation bottle. The mixture was shaken for 2 h, filtered through Celite (filter aid), and the filtrate was concentrated to afford the title compound (1.0 g, 1.32 mmol, yield>100%) which was used directly for the next step without further purification. ESI: m/z: calcd for C₃₅H₇₀N₃O₁₄ [M+H]⁺: 756.48, found 756.47.

### Example 165. Synthesis of (S)-tert-butyl 37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-oate

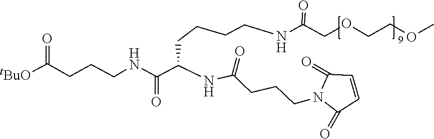

To a solution of (S)-tert-butyl 37-amino-31,38-dioxo-2,5,8,11,14,17,20,23,26,29-decaoxa-32,39-diazatritetracontan-43-oate (0.93 g, 1.23 mmol, 1.0 eq) and 4-(maleimidyl)butanoic acid (0.27 g, 1.47 mmol, 1.2 eq) in DMA (40 mL) at room temperature was added EDC (0.90 g, 4.68 mmol). The mixture was stirred overnight, then concentrated and diluted with water (50 mL) and extracted with DCM (80 mL×3), dried over anhydrous Na₂SO₄, filtered, concentrated and purified by SiO₂ column chromatography (DCM:MeOH=25:1) to give the title compound as a light yellow oil (1.01 g, 90%). ESI m/z: calcd for C₄₃H₇₇N₄O₁₇ [M+H]⁺: 921.5, found: 921.5.

### Example 166. Synthesis of (S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-oic Acid

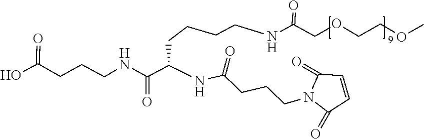

(S)-tert-butyl 37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-oate (0.90 g, 0.98 mmol) was dissolved in HCOOH (50 mL) and stirred at room temperature for 1 hour. The reaction mixture was concentrated and co-evaporated with toluene twice, and the residue was placed on a vacuum pump to the title compound (0.85 g, 0.98 mmol, crude product). ESI: m/z: calcd for C₃₉H₆₉N₄O₁₇ [M+H]⁺: 865.46, found 865.44.

### Example 167. Synthesis of (S)-2,5-dioxopyrrolidin-1-yl 37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-oate

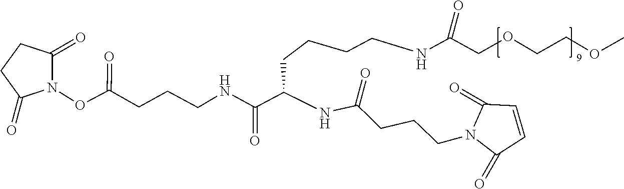

To a solution of (S)-37-(4-(2,5-dioxo-2,5-dihydro-H-pyrrol-1-yl)butanamido)-31,38-dioxo-2,5,8,11,14,17,20,23,26,29-decaoxa-32,39-diazatritetracontan-43-oic acid (0.80 g, 0.92 mmol, 1.0 eq) and 1-hydroxypyrrolidine-2,5-dione (NHS) (0.20 g, 1.73 mmol, 2.0 eq) in DMA (20 mL) at room temperature was added EDC (0.90 g, 4.68 mmol). The mixture was stirred overnight, then concentrated and purified by SiO₂ column chromatography (DCM:EtOAc=10:1 to 5:1) to give the title compound as a light yellow oil (0.803 g, 91%). ESI m/z: calcd for C₄₃H₇₂N₅O₁₉ [M+H]⁺: 962.47, found: 962.55.

### Example 168. Synthesis of a Cross-Linked PBD Dimer C-26

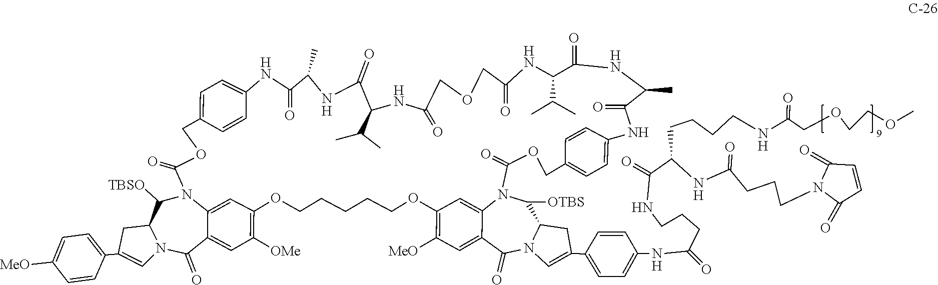

PBD dimer C-25 (120 mg, 0.068 mmol) and (S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-oic acid (70 mg, 0.0809 mmol) in DMA (3 ml) was added EDC (60 mg, 0.312 mmol). The mixture was stirred overnight, then concentrated and purified by SiO₂ column chromatography (DCM:EtOAc=10:1 to 5:1) to give the title compound as a foam (152 mg, 86%). ESI m/z: calcd for C₁₃₁H₁₈₆N₁₅O₃₆Si₂ [M+H]⁺: 2601.26, found: 2601.55.

### Example 168. Synthesis of a Cross-Linked PBD Dimer C-27

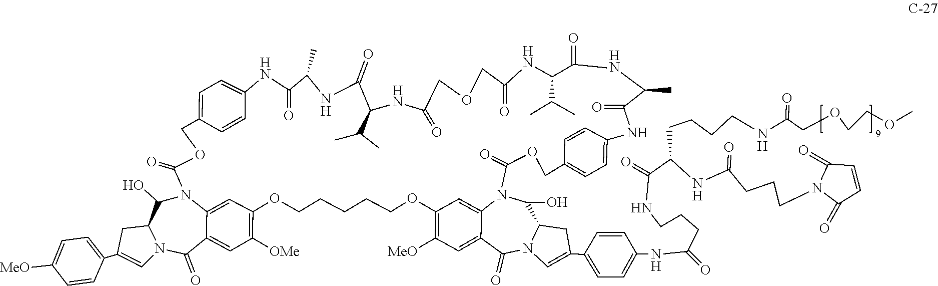

C-26 compound (75.5 mg, 0.0290 mmol) was dissolved in DCM (2 ml), followed by addition of TFA (2 ml) at 0-4° C. The reaction mixture was then stirred at RT for 1 h, diluted with toluene (5 ml), then concentrated, and co-evaporated with DCM/toluene to dryness to afford the crude product C-17 (72.2 mg, 105% yield, 91% pure by HPLC) which was further purified by reverse phase HPLC (250 (L) mm x 20(d) mm, C₁₈ column, 5-60% acetonitrile/water in 40 min, v=8 ml/min) to afford the pure product C-27 (55.2 mg, 80% yield, 95% pure) as a foam. ESI MS m/z: calcd for C₁₁₉H₁₅₈N₁₅O₃₆ [M+H]⁺ 2373.09, found 2373.90.

### Example 169. Synthesis of (S)-tert-butyl 13-(2-(((benzyloxy)carbonyl)amino)-5-(tert-butoxy)-5-oxopentanamido)tridecanoate

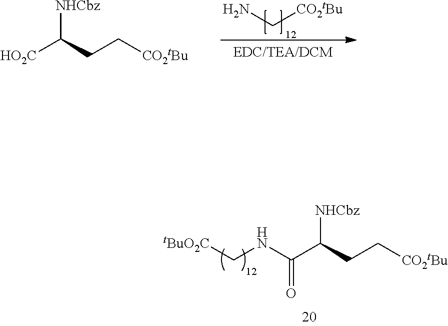

To the solution of (S)-2-(((benzyloxy)carbonyl)amino)-5-(tert-butoxy)-5-oxopentanoic acid (3.50 g, 10.38 mmol) and tert-butyl 13-aminotridecanoate (3.00 g, 10.51 mmol) in DMF (70 mL) was added EDC (10.00 g, 52.08 mmol) and TEA (1.60 mL, 11.16 mmol). The reaction was stirred at room temperature for 8 h, concentrated in vacuo, diluted with NaCl saturated water (80 ml) and EtOAc (100 ml), separated. The aqeuous layer was extracted with EtOAc (50 mL×3) and the combined organic phases were washed once with 100 mL of saturated brine, then dried over anhydrous Na₂SO₄, filtered and concentrated. The residue was purified by SiO₂ column chromatography (EtOAc/DCM, 1:15) to afford the title compound (5.45 g, 87% yield), ESI: m/z: calcd for C₃₄H₅₇N₂O₇ [M+H]⁺: 605.41, found 605.38.

### Example 170. Synthesis of (S)-tert-butyl 13-(2-amino-5-(tert-butoxy)-5-oxopentanamido)tridecanoate

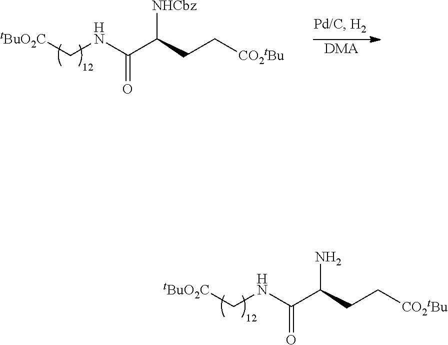

To a solution of S)-tert-butyl 13-(2-(((benzyloxy)carbonyl)amino)-5-(tert-butoxy)-5-oxopentanamido)tridecanoate (2.80 g, 4.63 mmol) in DMA (100 mL) was added 10% Pd/C (0.41 g), the mixture was stirred under hydrogen atmosphere at room temperature for 18 h. Then the Pd/C was removed by filtration through celite and the filter bed was washed with DMA. The filtrate was concentrated to afford the product as yellow foam which was used in the next step without further purification (2.19 g, 101% yield). ESI: m/z: calcd for C₂₆H₅₁N₂O₅[M+H]⁺: 471.37, found 471.80.

### Example 171. Synthesis of 2,2-dimethyl-4,17-dioxo-3,7,10,13,20,23,26-heptaoxa-16-azanonacosan-29-oic Acid

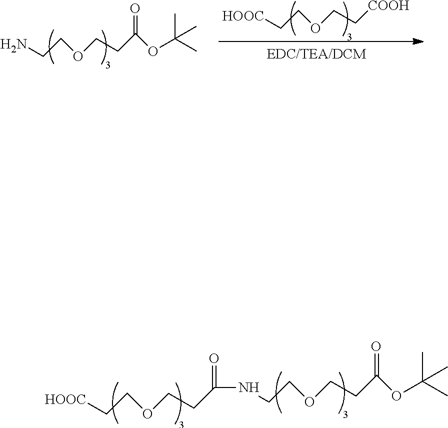

In a solution of tert-butyl 3-(2-(2-(2-aminoethoxy)ethoxy)ethoxy)propanoate (6.00 g, 21.64 mmol) and 3,3′-((oxybis(ethane-2,1-diyl))bis(oxy))dipropanoic acid (21.01 g, 84.00 mmol) in DMA (200 ml) were added EDC (18.00 g, 93.75 mmol) and DIPEA (5.00 g, 38.75 mmol). The mixture was stirred overnight, then concentrated and purified by SiO₂ column chromatography (MeOH:CH₂Cl₂=1:12 to 1:5) to give the title compound as a white oil (9.15 g, 86% yield). ESI m/z: calcd for C₂₃H₄₄NO₁₁ [M+H]⁺: 510.28, found: 510.55.

### Example 172. Synthesis of 1-benzyl 39-tert-butyl 14,26-dioxo-4,7,10,17,20,23,30,33,36-nonaoxa-13,27-diazanonatriacontane-1,39-dioate

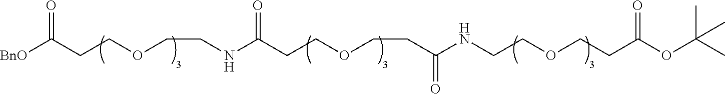

In a solution of (S)-tert-butyl 13-(2-amino-5-(tert-butoxy)-5-oxopentanamido)tridecanoate (5.11 g, 10.03 mmol) and benzyl 3-(2-(2-(2-aminoethoxy)ethoxy)ethoxy)propanoate (3.21 g, 10.31 mmol) in DMA (100 ml) were added EDC (8.02 g, 41.77 mmol) and DIPEA (3.00 g, 23.25 mmol). The mixture was stirred overnight, then concentrated and purified by SiO₂ column chromatography (EtOAc:CH₂Cl₂=1:8 to 1:3) to give the title compound as a white oil (7.01 g, 87% yield). ESI m/z: calcd for C₃₉H₆₇N₂O₁₅ [M+H]⁺: 803.44, found: 803.80.

### Example 173. Synthesis of 3,16,28-trioxo-1-phenyl-2,6,9,12,19,22,25,32,35,38-decaoxa-15,29-diazahentetracontan-41-oic Acid

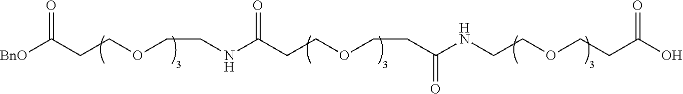

1-benzyl 39-tert-butyl 14,26-dioxo-4,7,10,17,20,23,30,33,36-nonaoxa-13,27-diazanonatriacontane-1,39-dioate (6.90 g, 8.60 mmol) was dissolved in HCOOH (50 mL) and stirred at 0-4° C. for 1 hour. The reaction mixture was diluted with toluene (50 ml), concentrated and co-evaporated with toluene twice, and the residue was placed on a vacuum pump to the title compound (6.45 g, ˜101% yield, crude product). ESI: m/z: calcd for C₃₅H₅₉N₂O₁₅ [M+H]⁺: 747.38, found 747.50.

### Example 174. Synthesis of 1-benzyl 39-(2,5-dioxopyrrolidin-1-yl) 14,26-dioxo-4,7,10,17,20,23,30,33,36-nonaoxa-13,27-diazanonatriacontane-1,39-dioate

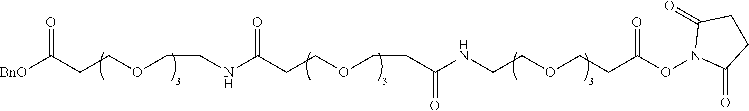

In a solution of 3,16,28-trioxo-1-phenyl-2,6,9,12,19,22,25,32,35,38-decaoxa-15,29-diazahentetracontan-41-oic acid (4.01 g, 5.37 mmol) and NHS (N-hydroxysuccinimde) (0.68 g, 5.91 mmol) in DMA (100 ml) were added EDC (1.52 g, 7.92 mmol) and DIPEA (0.50 g, 3.87 mmol). The mixture was stirred overnight, then concentrated and purified by SiO₂ column chromatography (EtOAc:CH₂Cl₂=1:8 to 1:4) to give the title compound as a white foam (4.17 g, 92% yield). ESI m/z: calcd for C₃₉H₆₂N₃O₁₇ [M+H]⁺: 844.40, found: 844.85.

### Example 175. Synthesis of (S)-47-(((benzyloxy)carbonyl)amino)-3,16,28,41-tetraoxo-1-phenyl-2,6,9,12,19,22,25,32,35,38-decaoxa-15,29,42-triazaoctatetracontan-48-oic Acid

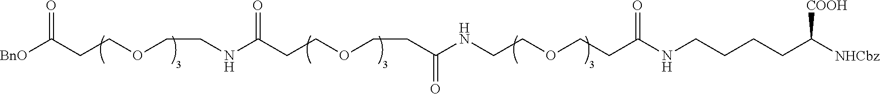

In a solution of (S)-6-amino-2-(((benzyloxy)carbonyl)amino)hexanoic acid (1.38 g, 4.92 mmol) in DMA (30 ml) and 100 mM NaH₂PO₄, pH 7.5 buffer (40 ml) was added 1-benzyl 39-(2,5-dioxopyrrolidin-1-yl) 14,26-dioxo-4,7,10,17,20,23,30,33,36-nonaoxa-13,27-diazanonatriacontane-1,39-dioate (4.15 g, 4.92 mmol) in 4 portions in 2 h. The mixture was stirred for 4 h, then concentrated and purified by SiO₂ column chromatography (MeOH:CH₂Cl₂=1:7 to 1:4) to give the title compound as a white foam (4.07 g, 82% yield). ESI m/z: calcd for C₄₉H₇₇N₄O₁₈ [M+H]⁺: 1009.51, found: 1009.90.

### Example 176. Synthesis of (S)-1-benzyl 51-(2-(trimethylsilyl)ethyl) 45-(((benzyloxy)-carbonyl)amino)-14,26,39,46-tetraoxo-4,7,10,17,20,23,30,33,36-nonaoxa-13,27,40,47-tetraazahenpentacontane-1,51-dioate

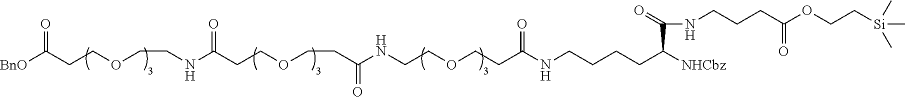

In a solution of (S)-47-(((benzyloxy)carbonyl)amino)-3,16,28,41-tetraoxo-1-phenyl-2,6,9,12,19,22,25,32,35,38-decaoxa-15,29,42-triazaoctatetracontan-48-oic acid (4.00 g, 3.96 mmol) and 2-(trimethylsilyl)ethyl 4-aminobutanoate (0.90 g, 4.43 mmol) in DMA (25 ml) was added EDC (2.03 g, 10.57 mmol). The mixture was stirred for 6 h, then concentrated and purified by SiO₂ column chromatography (MeOH:CH₂Cl₂=1:15 to 1:8) to give the title compound as a white foam (3.97 g, 84% yield). ESI m/z: calcd for C₅₈H₉₆N₅O₁₉Si [M+H]⁺: 1194.64, found: 1194.90.

### Example 177. Synthesis of 12-amino-2,2-dimethyl-6,11,18,31,43-pentaoxo-5,21,24,27,34,37,40,47,50,53-decaoxa-10,17,30,44-tetraaza-2-silahexapentacontan-56-oic Acid

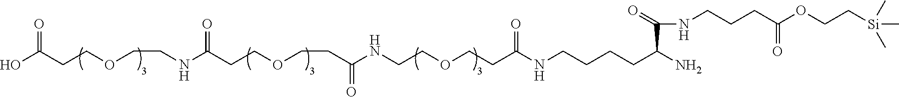

To a solution of (S)-1-benzyl 51-(2-(trimethylsilyl)ethyl) 45-(((benzyloxy)-carbonyl)amino)-14,26,39,46-tetraoxo-4,7,10,17,20,23,30,33,36-nonaoxa-13,27,40,47-tetraazahenpentacontane-1,51-dioate (3.90 g, 3.33 mmol) in MeOH (40 mL) was added Pd/C (10 wt %, 0.20 g) in a hydrogenation bottle. The mixture was shaken at 40 psi of H2 for 2 h, filtered through Celite (filter aid), and the filtrate was concentrated to afford the title compound (3.16 g, 98% yield) which was used directly for the next step without further purification. ESI: m/z: calcd for C₄₃H₈₃N₅O₁₇Si [M+H]⁺: 970.55, found 970.70.

### Example 178. Synthesis of 2,5-dioxopyrrolidin-1-yl 4-((3aR,7R,7aS)-1,3-dioxo-3a,4,7,7a-tetrahydro-1H-4,7-epoxyisoindol-2(3H)-yl)butanoate

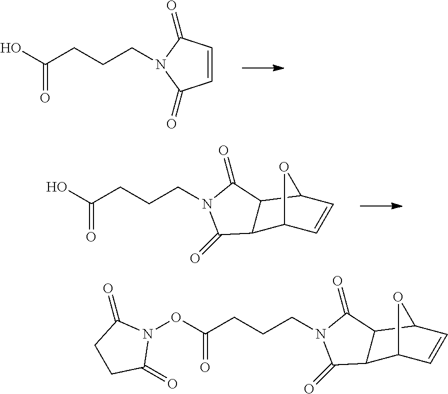 [SMILES: O=C(O)CCCN1C(=O)C=CC1=O ; O=C(O)CCCN1C(=O)C2C3C=CC(O3)C2C1=O ; O=C(CCCN1C(=O)C2C3C=CC(O3)C2C1=O)ON1C(=O)CCC1=O]

4-(2,5-dioxo-2,5-dihydro-1H-pyrrol-1-yl)butanoic acid (10.0 g, 54.62 mmol) and furan (5 ml, 68.74 mmol) in ether (90 ml) in a pressure vessel was heated at 170° C. for 6 h. Then the solution was cooled down to room temperature, concentrated in vacuo and crystalized in EtOH/Hexane to afford 4-((3aR,7R,7aS)-1,3-dioxo-3a,4,7,7a-tetrahydro-1H-4,7-epoxyisoindol-2(3H)-yl)butanoic acid (11.24 g, 44.76 mmol, 82% yield). Then the yield acid compound redissolved in CH₂Cl₂ (100 ml) was added NHS (7.00 g, 60.86 mmol) and EDC (25.00 g, 130.20 mmol). The mixture was stirred for 6 h, then concentrated and purified by SiO₂ column chromatography (EtOAc:CH₂Cl₂=1:8 to 1:5) to give the title compound as a white foam (13.57 g, 87% yield). ESI m/z: calcd for C₁₆H₁₇N₂O₇ [M+H]⁺: 349.09, found: 349.55.

### Example 179. Synthesis of (12S)-12-(4-((3aR,4S,7R)-1,3-dioxo-3a,4,7,7a-tetrahydro-1H-4,7-epoxyisoindol-2(3H)-yl)butanamido)-2,2-dimethyl-6,11,18,31,43-pentaoxo-5,21,24,27,34,37,40,47,50,53-decaoxa-10,17,30,44-tetraaza-2-silahexapentacontan-56-oic Acid

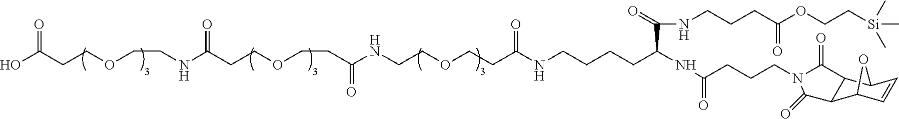

In a mixture of 12-amino-2,2-dimethyl-6,11,18,31,43-pentaoxo-5,21,24,27,34,37,40,47,50, 53-decaoxa-10,17,30,44-tetraaza-2-silahexapentacontan-56-oic acid (3.10 g, 3.19 mmol) in DMA (20 ml) and 100 mM NaH₂PO₄, pH 7.5 (20 ml) at 15° C. was added 2,5-dioxopyrrolidin-1-yl 4-((3aR,4S,7R)-1,3-dioxo-3a,4,7,7a-tetrahydro-1H-4,7-epoxyisoindol-2(3H)-yl)butanoate (1.60 g, 4.60 mmol) in DMA (10 ml). The mixture was stirred for 6 h, then concentrated and purified by SiO₂ column chromatography (MeOH:CH₂Cl₂=1:7 to 1:4) to give the title compound as a white foam (3.07 g, 80% yield). ESI m/z: calcd for C₅₅H₉₅N₆O₂₁Si [M+H]⁺: 1203.63, found: 1203.84.

### Example 180. Synthesis of (7S,53S)-68-tert-butyl 1-(2-(trimethylsilyl)ethyl) 53-(3-(tert-butoxy)-3-oxopropyl)-7-(4-((3aR,4S,7R)-1,3-dioxo-3a,4,7,7a-tetrahydro-1H-4,7-epoxyisoindol-2(3H)-yl)butanamido)-6,13,26,38,51,54-hexaoxo-16,19,22,29,32,35,42,45,48-nonaoxa-5,12,25,39,52,55-hexaazaoctahexacontane-1,68-dioate

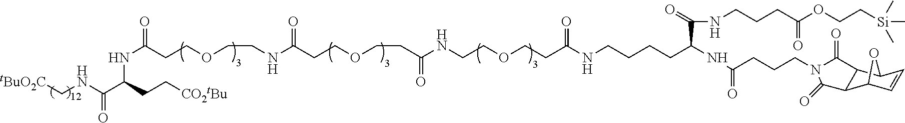

In a solution of (12S)-12-(4-((3aR,4S,7R)-1,3-dioxo-3a,4,7,7a-tetrahydro-1H-4,7-epoxyisoindol-2(3H)-yl)butanamido)-2,2-dimethyl-6,11,18,31,43-pentaoxo-5,21,24,27,34,37,40, 47,50,53-decaoxa-10,17,30,44-tetraaza-2-silahexapentacontan-56-oic acid (3.00 g, 2.49 mmol) and (S)-tert-butyl 13-(2-amino-5-(tert-butoxy)-5-oxopentanamido)tridecanoate (1.18 g, ˜2.49 mmol) in DMA (40 ml) was added EDC (2.03 g, 10.57 mmol). The mixture was stirred for 6 h, then concentrated and purified by SiO₂ column chromatography (EtOAc:CH₂Cl₂=1:10 to 1:4) to give the title compound as a white foam (3.50 g, 85% yield). ESI m/z: calcd for C₈₁H₁₄₃N₈O₂₅Si [M+H]⁺: 1655.98, found: 1655.90.

### Example 181. Synthesis of (19S,65S)-19-(3-(tert-butoxy)-3-oxopropyl)-65-(4-((3aR,4S,7R)-1,3-dioxo-3a,4,7,7a-tetrahydro-1H-4,7-epoxyisoindol-2(3H)-yl)butanamido)-2,2-dimethyl-4,18,21,34,46,59,66-heptaoxo-3,24,27,30,37,40,43,50,53,56-decaoxa-17,20,33,47,60,67-hexaazahenheptacontan-71-oic Acid

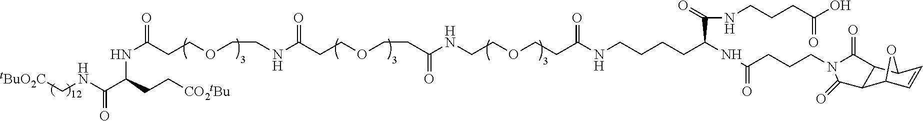

In a solution of (7S,53S)-68-tert-butyl 1-(2-(trimethylsilyl)ethyl) 53-(3-(tert-butoxy)-3-oxopropyl)-7-(4-((3aR,4S,7R)-1,3-dioxo-3a,4,7,7a-tetrahydro-1H-4,7-epoxyisoindol-2(3H)-yl)butanamido)-6,13,26,38,51,54-hexaoxo-16,19,22,29,32,35,42,45,48-nonaoxa-5,12,25,39,52,55-hexaazaoctahexacontane-1,68-dioate (3.40 g, 2.05 mmol) in THE (40 ml) was added TBAF (1.53 g, 5.74 mmol) in THE (10 ml). The mixture was stirred for 4 h, then concentrated and purified by SiO₂ column chromatography (MeOH:CH₂Cl₂=1:6 to 1:3) to give the title compound as a white foam (2.77 g, 87% yield). ESI m/z: calcd for C₇₆H₁₃₁N₈O₂₅Si [M+H]⁺: 1554.91, found: 1554.95.

### Example 182. Synthesis of Cross-Linked PBD Dimer C-28

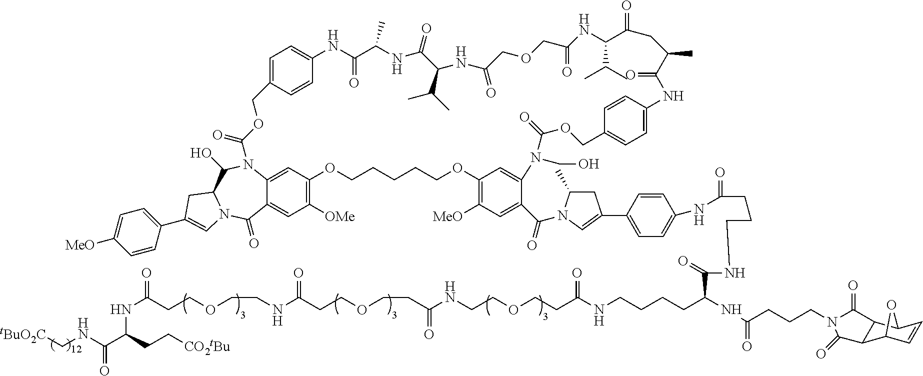

In a solution of (19S,65S)-19-(3-(tert-butoxy)-3-oxopropyl)-65-(4-((3aR,4S,7R)-1,3-dioxo-3a,4,7,7a-tetrahydro-1H-4,7-epoxyisoindol-2(3H)-yl)butanamido)-2,2-dimethyl-4,18,21,34,46,59,66-heptaoxo-3,24,27,30,37,40,43,50,53,56-decaoxa-17,20,33,47,60,67-hexaazahenheptacontan-71-oic acid (126 mg, 0.081 mmol) and PBD dimer C-25 (140 mg, 0.080 mmol) in DMA (10 ml) was added EDC (45 mg, 0.234 mmol). The mixture was stirred for 8 h, then concentrated and purified by SiO₂ column chromatography (EtOAc:CH₂Cl₂=1:8 to 1:3) to give the title compound as a white foam (195 mg, 79% yield). ESI m/z: calcd for C₁₅₆H₂₂₀N₁₉O₄₄ [M+H]⁺: 3063.55, found: 3063.90.

### Example 183. Synthesis of Cross-Linked PBD Dimer C-29

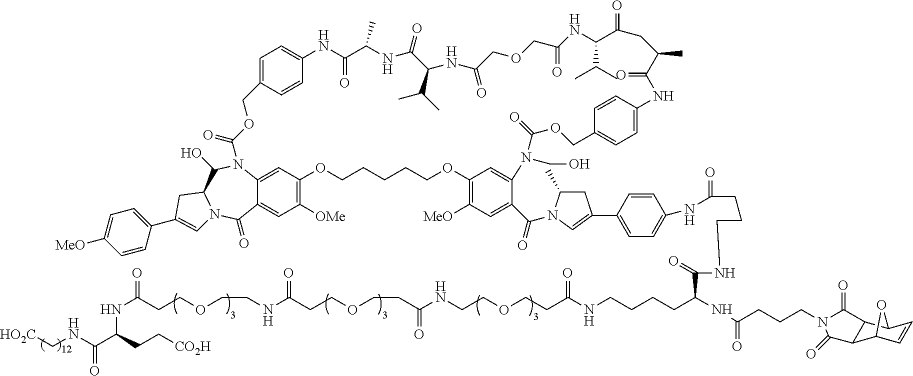

The cross-linked PBD dimer C-28 (180 mg, 0.0587 mmol) in the mixture solution of DMA (8 ml) and toluene (10 ml) was refluxed at 120° C. for 6 h, and LC-MS indicated that the furan was deprotected from the maleimide group. The solution was evaporated in vacuo, and redissolved in the mixture of 1,4-dioxane (6 ml) and 12 M HCl solution (2 ml). After stirred for 30 min, the mixture was concentrated and purified by reverse phase HPLC (250 (L) mm x 30(d) mm, C₁₈ column, 5-60% acetonitrile/water in 40 min, v=8 ml/min) to afford the pure product C-29 (143.2 mg, 83% yield, 95% pure) as a foam after lyophilized. ESI MS m/z: calcd for C₁₄₈H₂₀₈N₁₉O₄₃ [M+H]⁺ 2939.46, found 2939.90.

### Example 184. General Method of Preparation of Conjugates CC-3, CC-4, CC-5, CC-6, CC-7, CC-8, CC-10, CC-11, CC-12, CC-14, CC-15, CC-17, CC-18, CC-20, CC-21, CC-27, and CC-29 from the Cross-Linked PBD Dimer C-3, C-4, C-5, C-6, C-7, C-8, C-10, C-11, C-12, C-14, CC-1, C-17, C-18, C-20, C-21, C-27, and C-29 Respectively

To a mixture of 2.0 mL of 10 mg/ml Herceptin in pH 6.0-8.0, were added of 0.70˜2.0 mL PBS buffer of 100 mM NaH₂PO₄, pH 6.5-8.5 buffers, TCEP (14-35 μL, 20 mM in water) and the compound C-3, C-4, C-5, C-6, C-7, C-8, C-10, C-11, C-12, C-14, CC-1, C-17, C-18, C-20, C-21, C-27, or C-29 (14-28 μL, 20 mM in DMA, independently. The mixture was incubated at RT for 4-18 h, then DHAA (135 μL, 50 mM) was added in. After continuous incubation at RT overnight, the mixture was added maleimide (40 μL, 20 mM in DMA) and then continuously incubated for 2 more hours, purified on G-25 column eluted with 100 mM NaH₂PO₄, 50 mM NaCl pH 6.0-7.5 buffer to afford 12.8-18.1 mg of the conjugate compound CC-3, CC-4, CC-5, CC-6, CC-7, CC-8, CC-10, CC-11, C-12, C-14, CC-1, C-17, C-18, C-20, C-21, C-27, or C-29, (83%94% yield) accordingly in 13.6˜15.8 ml buffer. The PBD dimer drug/antibody ratios (DAR) were 3.6˜4.1 determined via UPLC-QTOF mass spectrum. The conjugates were 95˜99% monomer analyzed by SEC HPLC (Tosoh Bioscience, Tskgel G3000SW, 7.8 mm ID x 30 cm, 0.5 ml/min, 100 min) and a single band measured by SDS-PAGE gel. The conjugate structures are shown below:

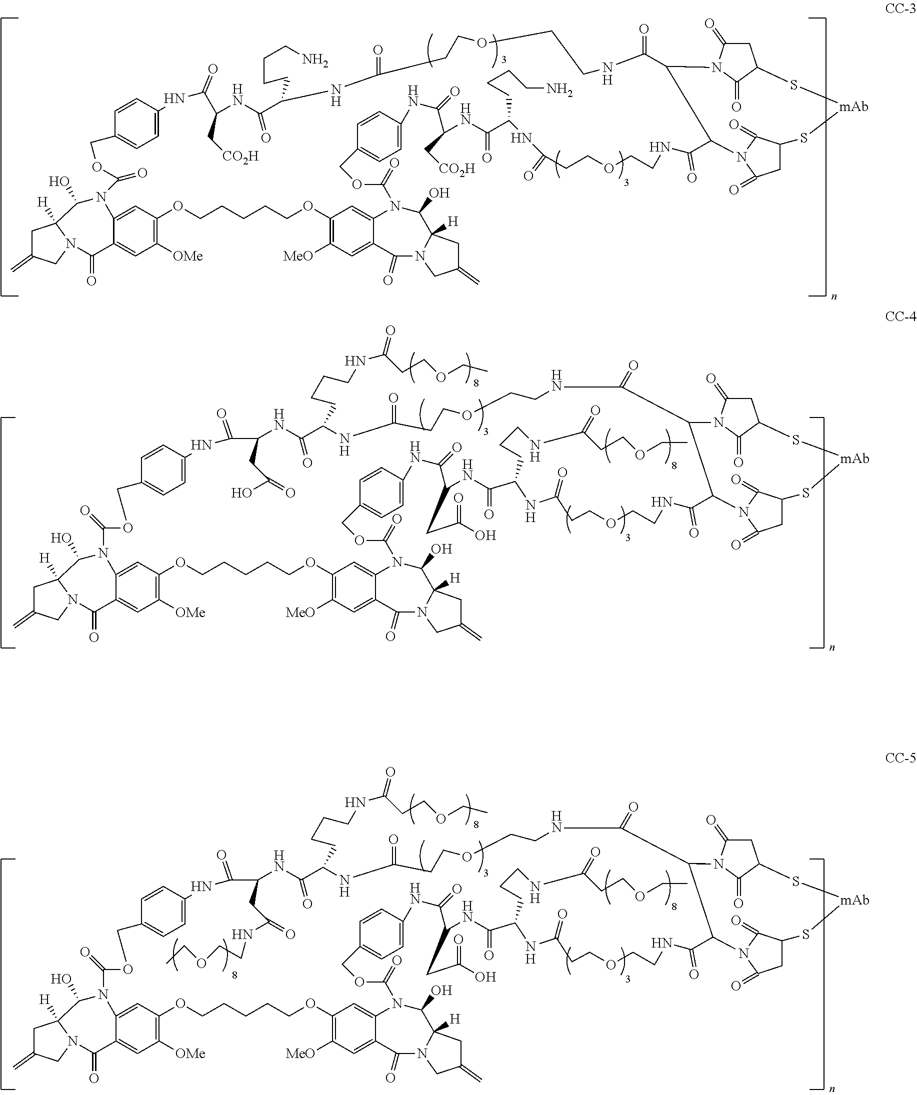

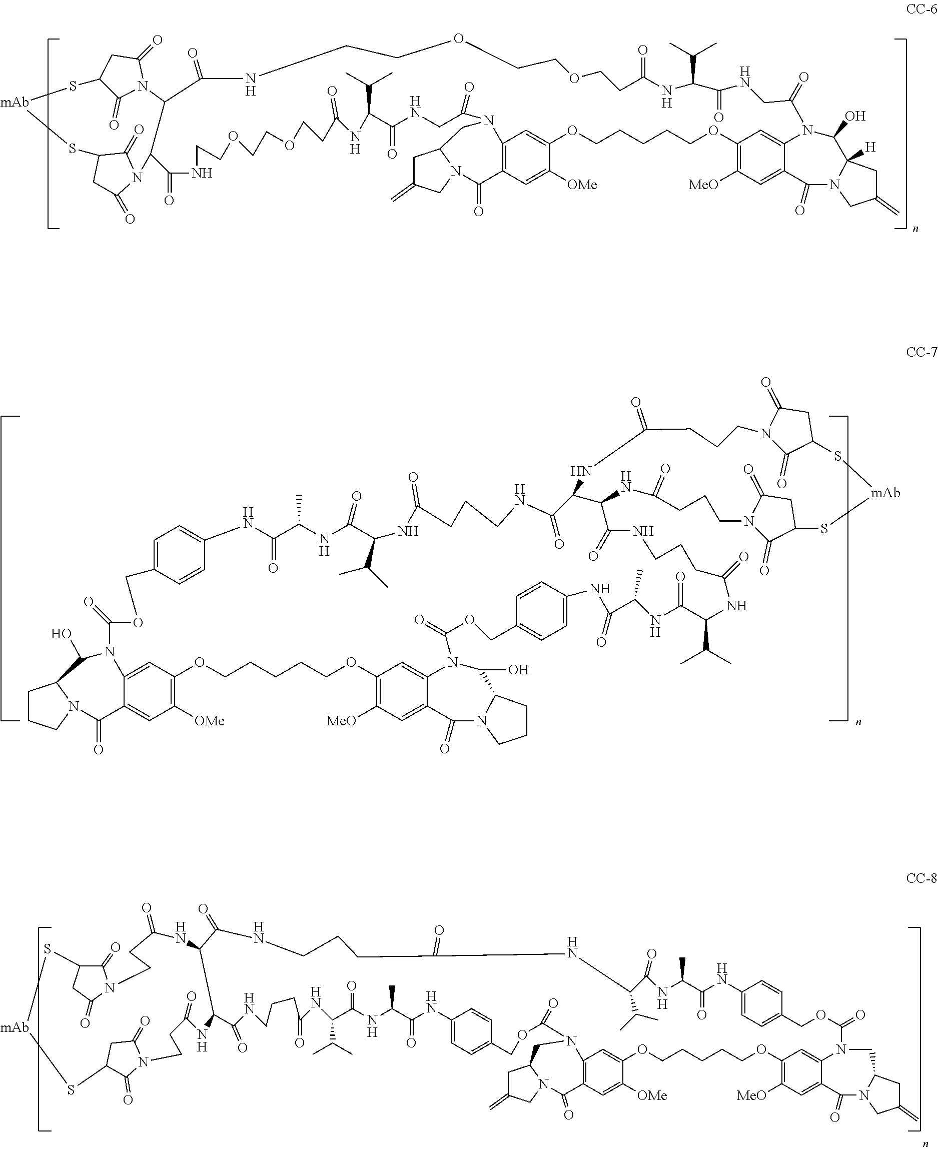

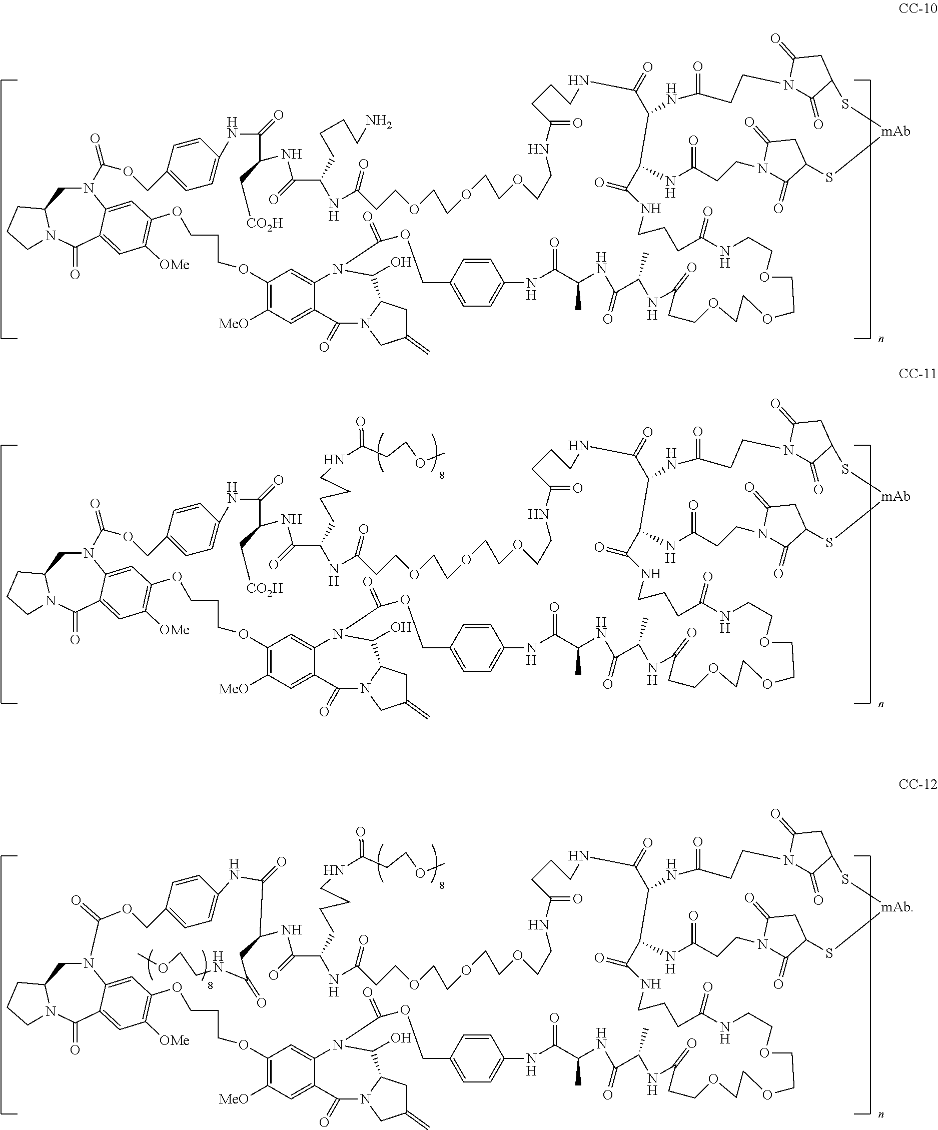

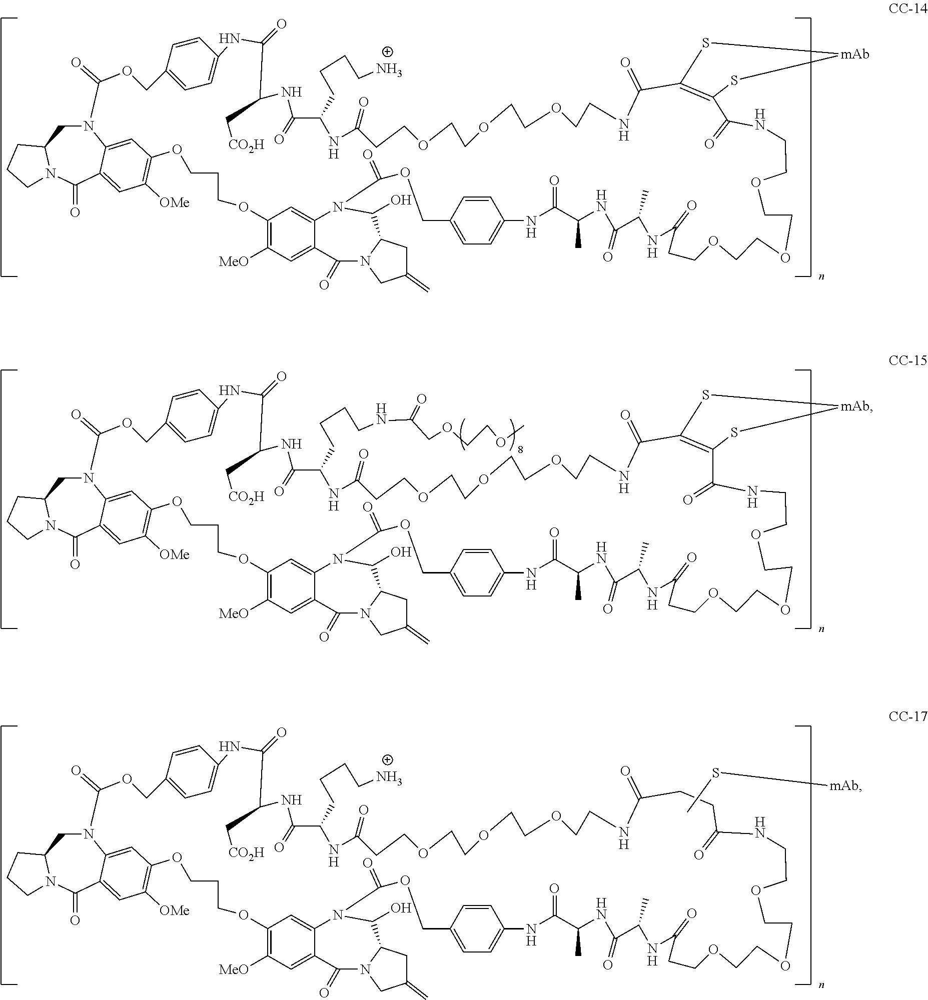

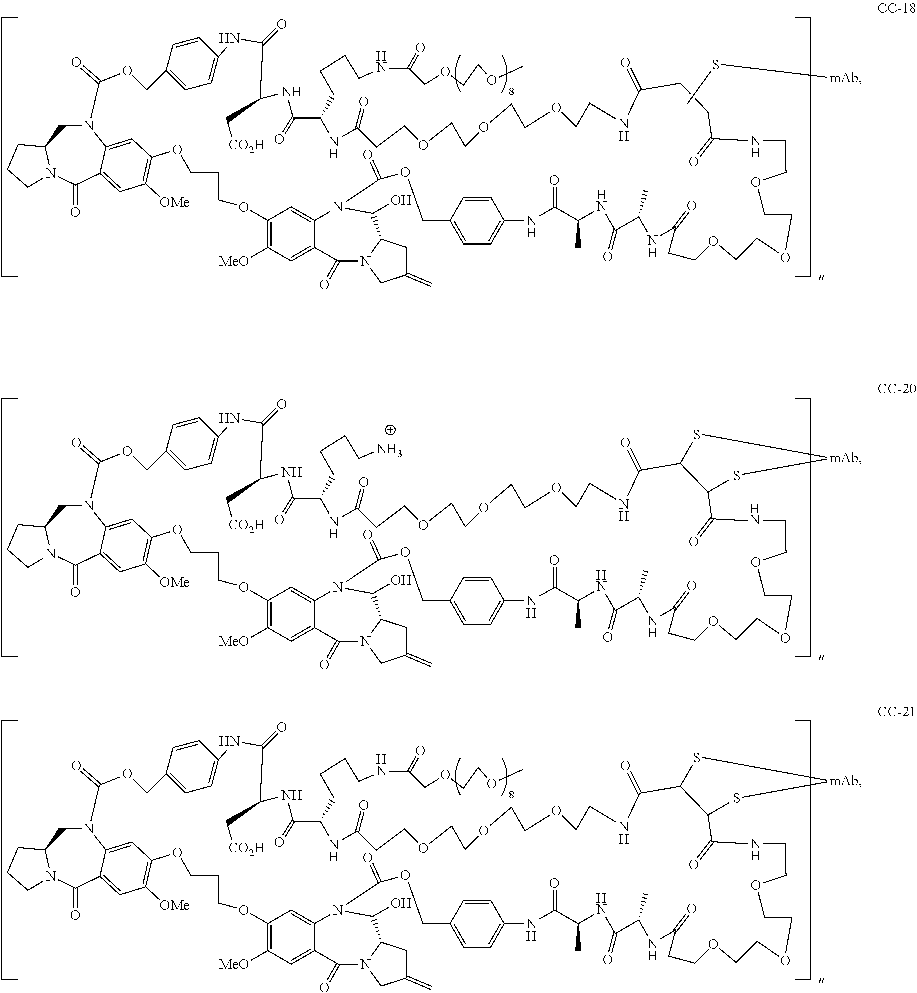

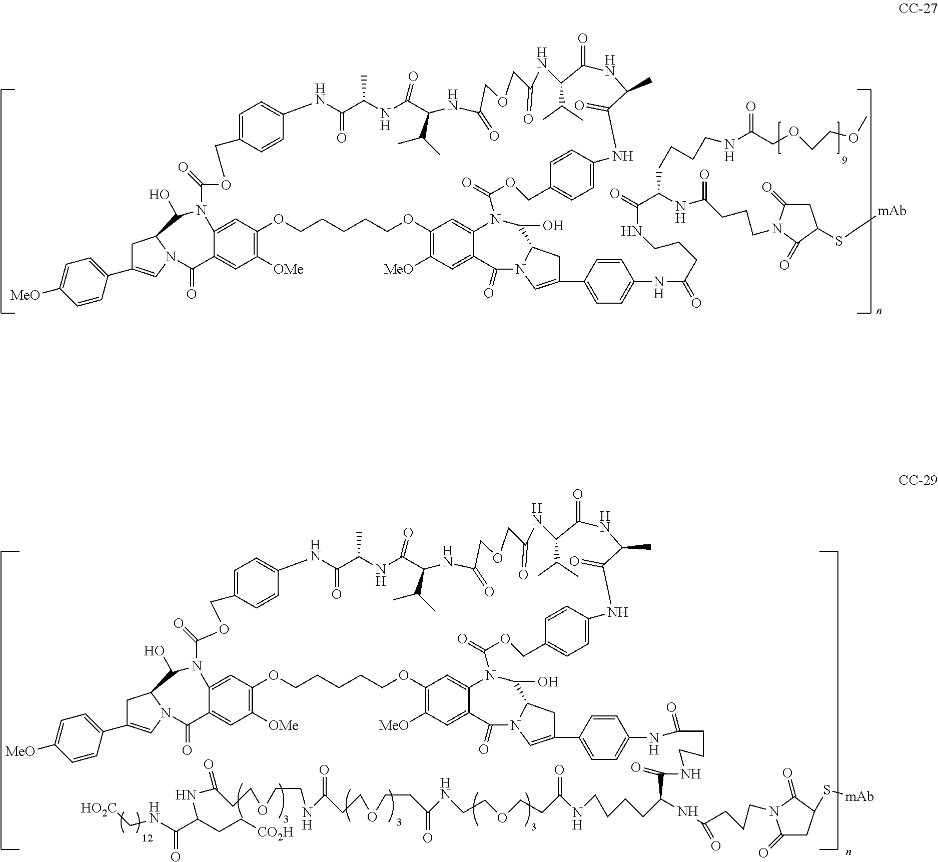

### Example 185. In Vitro Cytotoxicity Evaluation of Conjugates CC-3, CC-4, CC-5, CC-6, CC-7, CC-8, CC-10, CC-11, CC-12, CC-14, CC-15, CC-17, CC-18, CC-20, CC-21, CC-27, and CC-29, in Comparison with T-DM1

The cell line used in the cytotoxicity assays was NCI-N87, a human gastric carcinoma cell line; The cells were grown in RPMI-1640 with 10% FBS. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 5% CO₂. MTT (5 mg/ml) was then added to the wells (20 μl) and the plates were incubated for 1.5 hr at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation: inhibition %=[1−(assay-blank)/(control-blank)]×100.

The cytotoxicity results of IC₅₀:

| | DAR (drug/ | N87 cell (Ag+) |
|---|---|---|
| | mAb ratio) | IC₅₀ (nM) |
| Conjugate CC-3 | 4.1 | 0.0172 nM |
| Conjugate CC-4 | 3.8 | 0.0397 nM |
| Conjugate CC-5 | 4.0 | 0.104 nM |
| Conjugate CC-6 | 3.8 | 1.132 nM |
| Conjugate CC-7 | 3.8 | 0.0212 nM |
| Conjugate CC-8 | 3.8 | 61.36 nM |
| Conjugate CC-10 | 3.9 | 0.133 nM |
| Conjugate CC-11 | 3.8 | 0.431 nM |
| Conjugate CC-12 | 3.6 | 0.716 nM |
| Conjugate CC-14 | 3.9 | 0.997 nM |
| Conjugate CC-15 | 4.0 | 0.504 nM |
| Conjugate CC-17 | 3.5 | 1.231 nM |
| Conjugate CC-18 | 3.5 | 0.821 nM |
| Conjugate CC-20 | 3.9 | 2.036 nM |
| Conjugate CC-21 | 3.9 | 0.693 nM |
| Conjugate CC-27 | 3.6 | 0.731 nM |
| Conjugate CC-29 | 3.6 | 1.115 nM |
| T-DM1 | 3.5 | 0.165 nM |

### Example 186. Antitumor Activity In Vivo (Balb/c Nude Mice Bearing nci-n87 Xenograft Tumor)

The in vivo efficacy of conjugates CC-2, CC-3, CC-4, CC-5, CC-6, CC-7, CC-10, CC-11, CC-12, CC-18 and CC-29 along with T-DM1 were evaluated in a human gastric carcinoma N-87 cell line tumor xenograft models. Five-week-old female BALB/c Nude mice (78 animals) were inoculated subcutaneously in the area under the right shoulder with N-87 carcinoma cells (5×10⁶ cells/mouse) in 0.1 mL of serum-free medium. The tumors were grown for 8 days to an average size of 130 mm³. The animals were then randomly divided into 13 groups (6 animals per group). The first group of mice served as the control group and was treated with the phosphate-buffered saline (PBS) vehicle. 12 groups were treated with conjugates CC-2, CC-3, CC-4, CC-5, CC-6, CC-7, CC-10, CC-li, CC-12, CC-18 and CC-29 and T-DM1 respectively at dose of 3 mg/Kg administered intravenously. Three dimensions of the tumor were measured every 4 days and the tumor volumes were calculated using the formula tumor volume=½(length×width×height). The weight of the animals was also measured at the same time. A mouse was sacrificed when any one of the following criteria was met: (1) loss of body weight of more than 20% from pretreatment weight, (2) tumor volume larger than 2000 mm³, (3) too sick to reach food and water, or (4) skin necrosis. A mouse was considered to be tumor-free if no tumor was palpable.

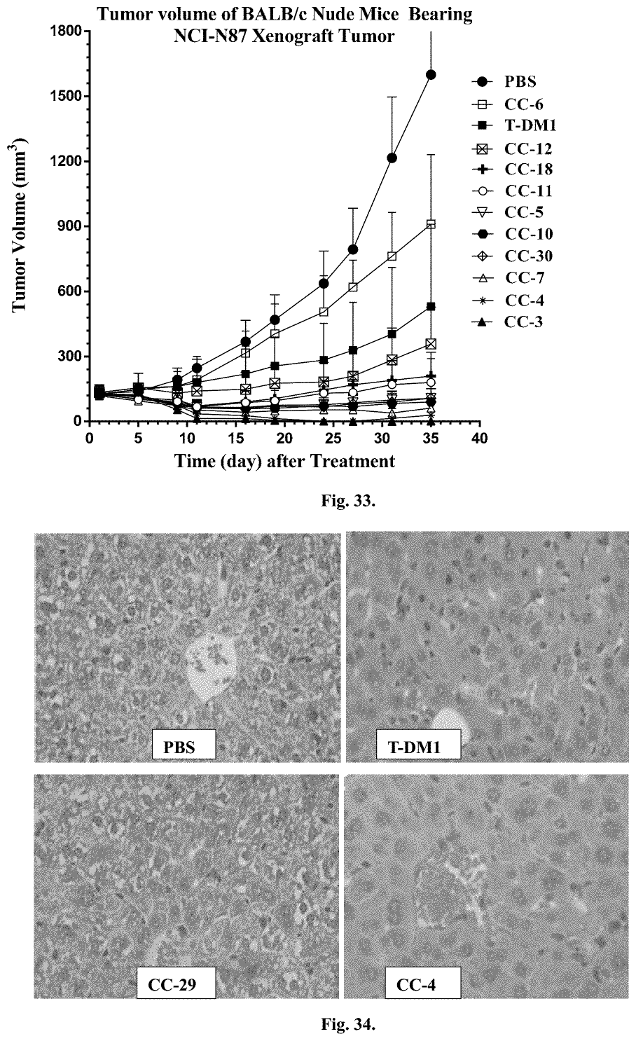

The results were plotted in FIG. 33. All the 12 conjugates did not cause the animal body weight loss. And the animals at control group were sacrificed at day 35 due to the tumor volume larger than 2000 mm³ and some of them were too sick. Here 9 conjugates except CC-6 tested demonstrated better anti-tumor activity than T-DM1. All 6/6 animals at the groups of compounds CC-4 and CC-3 had completely no tumor measurable at day 14 till day 30. In contrast T-DM1 at dose of 3 mg/Kg was not able to eliminate the tumors.

### Example 187. Animal Liver Toxicity Study of the Conjugate CC-4, CC-29 at a High Dose in Comparison with T-DM1 in ICR Mice

Twenty ICR mice were then randomly divided into 4 groups (5 animals per group) and each animal was given 75 mg/Kg of the conjugates (PBS, CC-4, CC-29 and T-DM1) in a single I.V. injection accordingly. Five days after doing, a 150 μl blood sample was collected from the retro-orbital orbital venous plexus (sinus) of each mouse. After centrifugation of the blood samples, the serum was taken to assay AST and ALT levels according to test kits from BioSino Bio-Technology and Science Inc (Beijing, China). The mean values of AST and ALT from 5 animals per group are listed below. After mice were sacrificed, their liver tissues were collected, fixed in 10% neutral formalin solution, dehydrated with a sequence of ethanol solutions, and embedded in paraffin. The sections (5 m-thick) were cut, transferred onto glass slides, and stained with hematoxylin and eosin (H&E) (Kiernan J A (2008) Histological and Histochemical Methods: Theory and Practice. 4th ed. Bloxham, UK: Scion; Gomori, Sheehan and Hrapchak in: Histotechnology A Self-Instructional Text, ASCP Press. American Society of Clinical Pathologists Chicago 1990). The stained samples were examined using a light microscope (Nikon Eclipse TE2000-U, Tokyo, Japan) and photographed at 200× magnification.

As shown below, both CC-4 and T-DM1 at dose of 75 mg/Kg elevated serum AST and ALT, to much higher levels than those in CC-29 group.

| | PBS | CC-4 | CC-29 | T-DM1 |
|---|---|---|---|---|
| AST | 27 ± 11 | 2550 ± 430 | 578 ± 83 | 1350 ± 460 |
| | (IU/L) | (IU/L) | (IU/L) | (IU/L) |
| ALT | 38 ± 16 | 383 ± 45 | 147 ± 28 | 305 ± 133 |
| | (IU/L) | (IU/L) | (IU/L) | (IU/L) |

In FIG. 34, both CC-4 and T-DM1 groups displayed hepatocyte hydrogenesis, Kupffer's cell hyperplasia and focal necrosis, but CC-29 group did not show obvious differences from the control PBS group. Thus both FIG. 33 and FIG. 34 demonstrated that CC-29 conjugate has much wider therapeutical window (with much higher anti-tumor activity and much lower side toxicity in vivo) than T-DM1.

1. A cross-linked PBD dimer derivative or its conjugate to a cell-binding molecule having the structure of Formula (I): 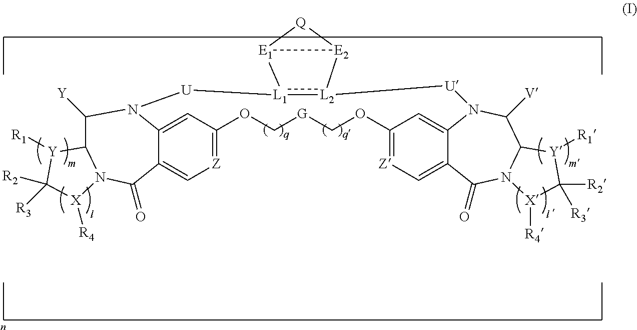
or their pharmaceutically acceptable salts, hydrates, or hydrated salts, or the polymorphic crystalline structures of these compounds or their optical isomers, racemates, diastereomers or enantiomers;
wherein:
----- represents an optional single bond or can be absent; [symbol]
represents an optional single bond or a double bond;
V and V′, are the same or different, and independently selected from the group consisting of H, OH, —NHOH; OR₅ (an ether); OCOR₅ (an ester); OCOOR₅ (a carbonate); NR₅R₅′, NR₅COR₅′, or NR₅NR₅′NR₅″ (an amine); OCONR₅R₅′ (a carbamate); NR₅(C═NH)NR₅′R₅″ (a guanidinum); NR₅CONR₅′R₅″ (a urea); OCSNHR₅ (a thiocarbamate); —SH (a thiol); —SR₅ (a sulfide); SOR₅ a sulphoxide (a sulphoxide); SOOR₅ (a sulfone); SO₃, HSO₃, HSO₂, or a salt of HSO³⁻, SO₃²⁻ or —HSO₂⁻ (a sulphite); OSO₃ (a bisulphite); NR₅SOOR₅′ (a sulfonamide); H₂S₂O₅ or a salt of S₂O₅²⁻ (a metabisulfite); PO₃SH₃, PO₂S₂H₂, POS₃H₂, PS₄H₂ or a salt of PO₃S³⁻, PO₂S₂³⁻, POS₃³⁻, PS₄³⁻ (mono-, di-, tri-, and tetra-thiophosphate); (R₅O)₂POSR₅′ (a thiophosphate ester); HS₂O₃ or a salt of S₂O₃²⁻ (a thiosulfate); HS₂O₄ or a salt of S₂O₄²⁻ (a dithionite); P(═S)(OR₅)(S)(OH) (a phosphorodithioate) or a salt thereof form with a cation; —NR₅OR₅′ (a hydroxylamine derivative); R₅C(═O)NOH (a hydroxamic acid) or a salt formed with a cation; HOCH₂SO₂—, or its salts (a formaldehyde sulfoxylate); NR₅COR₅, (an amide); O-glycoside; N₃ (an azido); CN (a cyano); X (a halo: F, Cl, Br, or I); C(R₅)(R_{5′})(R_{5″}) (a trialkyl), OP(O)(OR₅)(NHR_{5′}) or OP(O)(NHR₅)(NHR_{5′}) (a phosphoramidate (phosphoramidic acid), or P(R₅)(R_{5′})(R_{5″}) triarylphosphonium; Aa (an amino acid), or NR₅CO(Aa)ₜ (a peptide-), wherein Aa is an amino acid or a polypeptide containing between t=1-100 amino acid units; an aminoacid-derived group: an α-, β-, γ-, or ω-aminoacid, or an unnatural aminoacid; wherein R₅, R₅′ and R₅″ are described below;
l, m, q, l′, m′ and q′ are independently a number of 0, 1, 2, 3, 4, or 5; n is 1-30;
X, X′, Y and Y′ the same or different, independently, represent N, O, S, an alkyl, such as CH₂ or CHR₅, an alkene, such as ═CH— or ═CR₅—, an ether, such as —C(OR₅)H—;
Z and Z′ the same or different, independently, represent N, CH, CR₅, COH, CNH₂, CNHR₅, or COR₅, or Z and Z′ link together with —COR₅OC—; wherein R₅ is independently selected from C₁-C₈ alkyl and aryl;
G is —CH₂—, O, —N(R₅)—, S, —P(O)(OR₅)—, —P(O)(NR₅R_{5′})—, 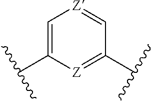
Z wherein Z and Z′ are defined above;
U and U′ are independently C(O), C(O)O, C(O)NH, C(O)N(R₅), C(═NH), C(═NH)O, C(═NH)NH, C(═NH)N(R₅), —C═N—, C(═S), C(O)S, C(S)NH, C(S)N(R₅), S(O), S(O)O, S(O)NH, S(O)(OR₅), S(O)(N(R₅)), S(O₂), S(O₂)O, P(O)(OR₅), P(O)(OR₅)O, P(O)(NH₂), P(O)(NR₅R_{5′}), P(O)(OR₅)NH—, P(O)(OR₅)NR_{5′}—, P(O)(N(R₅R_{5′})(N(R₅), P(S)(OR₅), P(S)(OR₅)O, P(S)(NH₂), P(S)(NR₅R_{5′}), P(S)(OR₅)NH—, P(S)(OR₅)NR_{5′}—, P(S)(N(R₅R_{5′})N(R₅), R₅, R₅O;
E₁ and E₂ are independently S, R₅S, C(O)S, C(O)NH, C(O)O, C(O)R₅S, C(═NH)NH, C(═NH)N(R₅), C(═NH)S, —C═N—, C(═S)S, C(O)S, C(═S)NH, C(═S)N(R₅), Ar—S, NC(O)CH₂S, ArC(O)CH₂S, S—S, 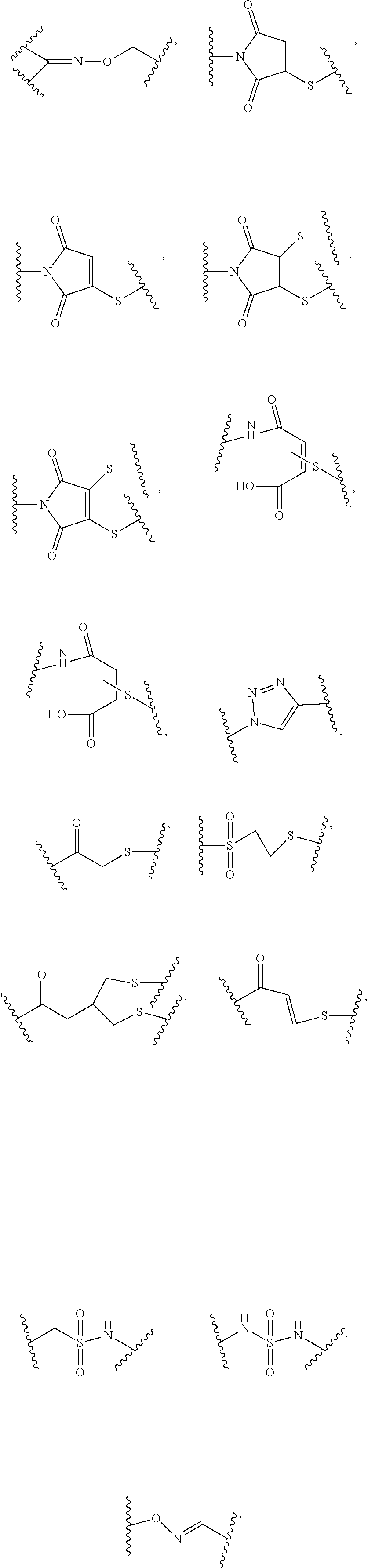
wherein the chemical bond in the middle of two atoms means it can link either of the adjoining two atoms; wavery line is a connecting site;
L₁, and L₂ are independently a releasable linker, or a linker which has a functional group on the linker that enables reaction with a cell-binding agent (CBA). L₁, and L₂ have independently the formula of: -Ww-(Aa)r-Tt-; or -Ww-(Aa)r-Tt-Q; or Q-Ww-(Aa)r-Tt-; wherein: —W— is a Stretcher unit; w is 0 or 1; -Aa- is independently an Amino Acid unit; r is independently an integer ranging from 0 to 100; -T- is a Spacer unit, which is a linear alkyl or branched alkyl, or polyethylene glycol spacer; and t is 0, or 1-100; the Stretcher unit W may independently contain a self-immolative spacer, peptidyl units, a hydrazone bond, a disulfide, an ester, or a thioether bond; w is 1 or 2 or 3; the Stretcher unit (—W—), when present, may link a targeted binding molecular unit (CBA) to an amino acid unit (--Aa--), or links T when an Aa is not present; the Stretcher unit W may independently contain a self-immolative spacer, peptidyl units, a hydrazone bond, disulfide or thiolether bonds; the cell-binding molecule (CBA) has a functional group that can form a bond with a functional group of a Stretcher; the functional group includes, sulfhydryl (—SH), amino, hydroxyl, carbonyl, oxyamino, alkynyl, heteroaromatic, an anomeric hydroxyl group of a carbohydrate, and carboxyl; L₁, and L₂ are independently selected from O, NH, N, S, P, NNH, NHNH, N(R₃), N(R₃)N(R_{3′}), CH, CO, C(O)NH, C(O)O, NHC(O)NH, NHC(O)O, polyethyleneoxy unit of formula (OCH₂CH₂)ₚOR₃, or (OCH₂CH(CH₃))ₚOR₃, or NH(CH₂CH₂O)PR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, -or CH₂CH₂(OCH₂CH₂)ₚCOOR₃, wherein p and p′ are independently an integer selected from 0-to about 1000, or combination thereof; C₁-C₈ alkyl, amide, amines, imines, hydrazines, hydrazones; C₂-C₈ heteroalkyl, alkylcycloalkyl, ethers, esters, hydrazones, ureas, semicarbazides, carbazides, alkoxyamines, alkoxylamines, urethanes, amino acids, peptides, acyloxylamines, hydroxamic acids, or heterocycloalkyl; C₃-C₈ aryl, Ar-alkyl, heterocyclic, carbocyclic, cycloalkyl, heteroalkylcycloalkyl, alkylcarbonyl, or heteroaryl; polyethyleneoxy unit of formula (OCH₂CH₂)ₚOR₃, or (OCH₂CH(CH₃))ₚOR₃, or NH(CH₂CH₂O)ₚR₃, or NH(CH₂CH(CH₃)O)ₚR₃, or N[(CH₂CH₂O)ₚR₃][(CH₂CH₂O)_{p′}R₃′], or (OCH₂CH₂)ₚCOOR₃, or CH₂CH₂(OCH₂CH₂)ₚCOOR₃, wherein p and p′ are independently an integer selected from 0 to about 5000, or combination thereof; wherein R₃ and R₃′ are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ esters, ether, or amide; or 1-8 amino acids; or polyethyleneoxy having formula (OCH₂CH₂)ₚ or (OCH₂CH(CH₃))ₚ, wherein p is an integer from 0 to about 5000, or combination above thereof; or (Aa)ᵣ, r=1-12 (one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit; the term releasable linker includes at least one bond that can be broken under physiological conditions: a pH-labile, acid-labile, base-labile, oxidatively labile, metabolically labile, biochemically labile, or enzyme-labile bond;
R₁, R₂, R₃, R₄, R₁′, R₂′, R₃′, and R₄′ are the same or different and independently selected from —H, substituted linear, branched or cyclic alkyl, alkenyl or alkynyl having from 1 to 10 carbon atoms, —(OCH₂CH₂)ₜR₅ (-a polyethylene glycol unit), -halogen, NH(C═NH)NH₂ (a guanidinium), —OR₅, —NR₅R₅′, —NO₂, —NCO, —NR₅COR₅′, —SR₅, —SOR₅ (a sulfoxide), —SO₂R₅ (a sulfone), —SO₃⁻M⁺ or —SO₃H (a sulfonate), —OSO₃⁻M⁺ or OSO₃H (a sulfate), —SO₂NR₅R₅′ (a sulfonamide), CN (a cyano), N₃ (an azido), —COR₅, —OCOR₅, —OCONR₅R₅′, CF₃, OR₅, Aryl, heterocycle, or P(O)R₅R₅′R₅″;
R₅, R₅′ and R₅″ are independently selected from H, C₁-C₈ of alkyl, alkenyl, alkinyl, heteroalkyl, aryl, arylalkyl, carbonyl, or pharmaceutical salts;
R₁ and R₂ optionally join together, or R₁′ and R₂′ join together to form a ═O (ketone), ═S, ═NR, —C(═O)R, or a double bond containing group ═CR₅R₅′; and R₁ and R₂ join together, or R₁′ and R₂′ join together, or R₃ and R₄ join together, or R₃′ and R₄′ join together to form a C₃-C₁₂ aromatic, heterocyclic, carbocyclic, or heteroaryl ring;
Q is a cell binding molecule (CBA), or a functional group that enables reaction with a cell-binding agent, or a functional group capable of reacting with a linker attached on a cell binding agent; the function group is chosen from a thiol, an amine, a hydrazine, an alkoxylamino, a disulfide substituent, a maleimido, a haloacetyl group, an N-hydroxy succinimide ester, a keton, an ester, an aldehyde, an alkynyl, an alkenyl, or protected thiol or disulfide group, SAc, SSR₁ or SSAr, wherein Ar is aromatic group or hetero aromatic group; the cell-binding molecule is selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), a binding peptide, protein, or small molecule attached on albumin, a polymer, a dendrimer, a liposome, a nanoparticle, a vesicle, and a (viral) capsid;
U, U′, L₁, L₂, L′, E₁ or E₂ may independently be composed of one or more following components as shown below: 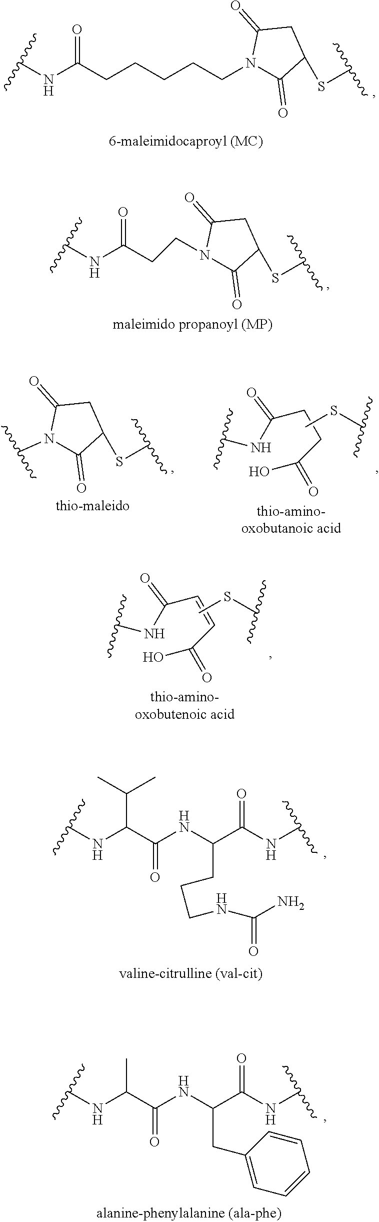 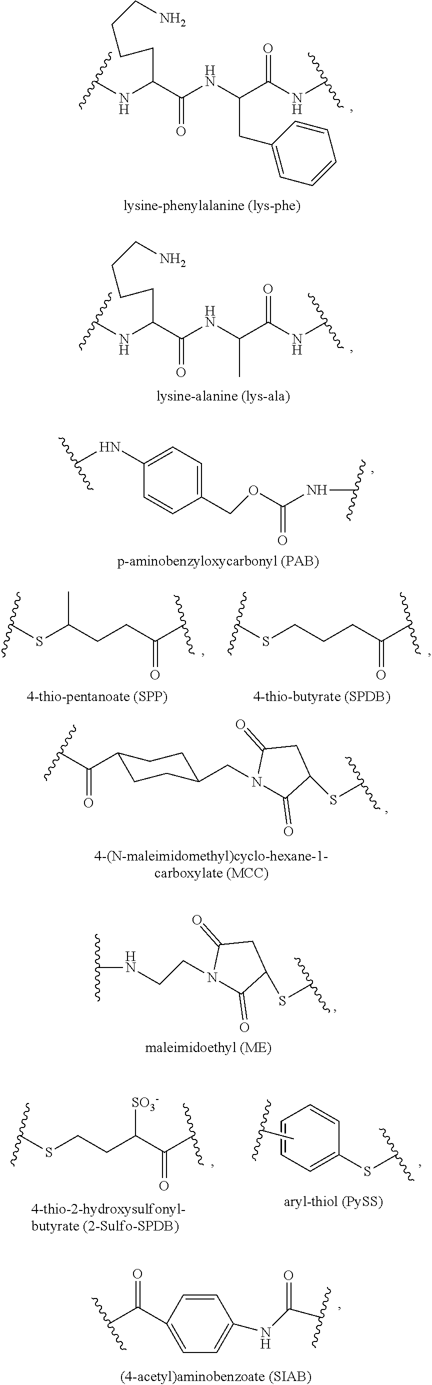 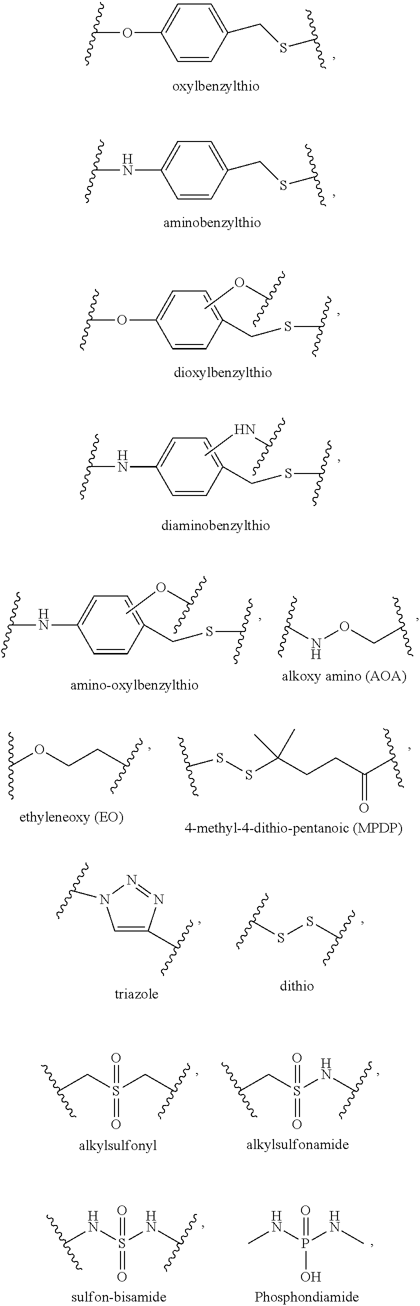 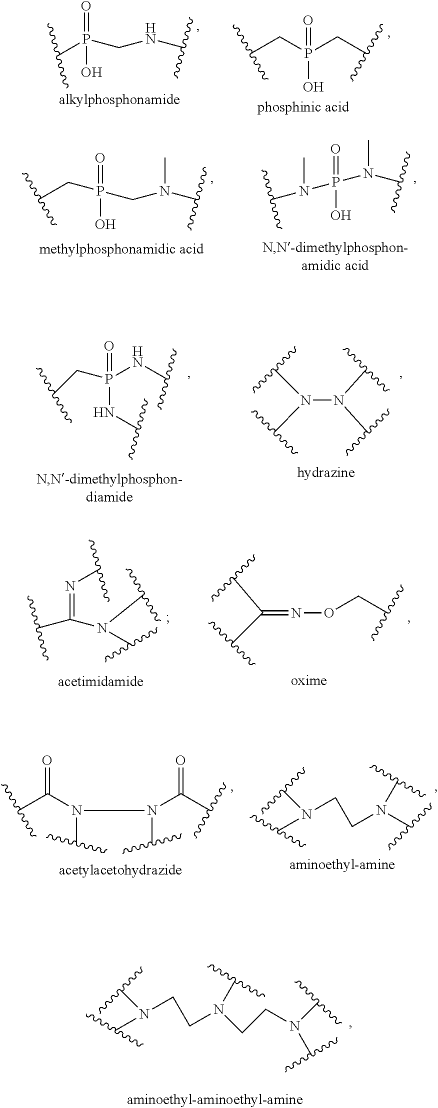
and L- or D-, natural or unnatural peptides containing 1-20 of the same or different amino acids; wherein a connecting bond in the middle of atoms means that it can connect either neighbor carbon atom bonds; wavery line is the site wherein another bond can be connected to;
or U, U′, E₁, or E₂, can be independently absent.

2. The cross-linked PBD dimer derivative or its conjugate according to claim 1 having the Formula (Ia), (Ib), (Ic), or (Ie): 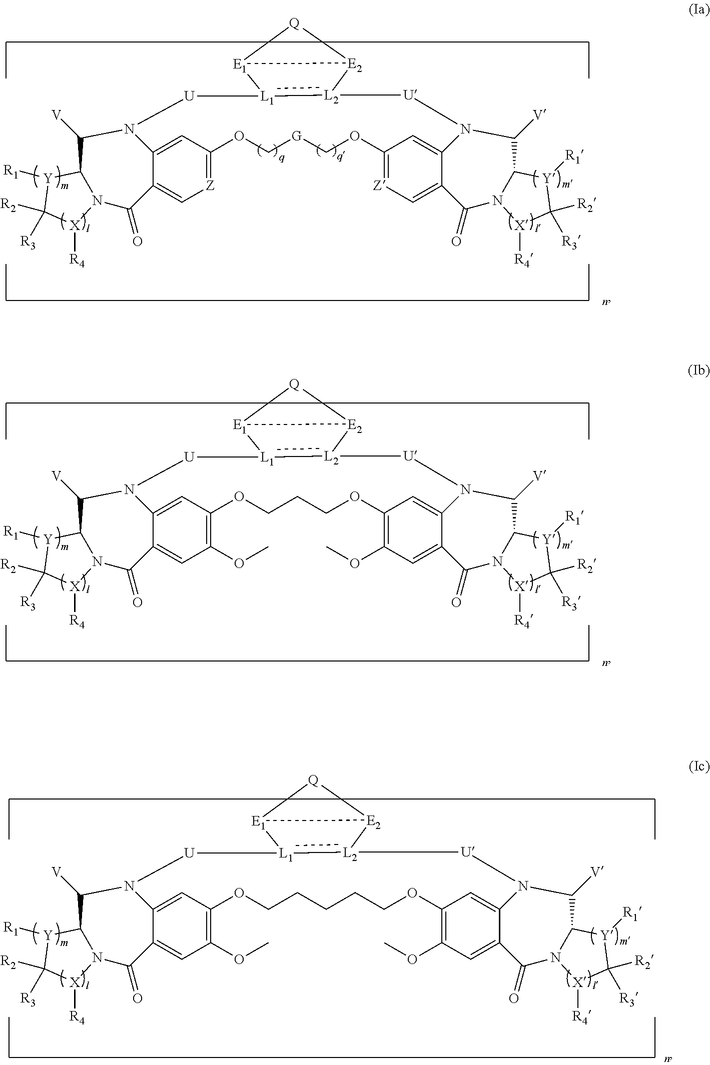 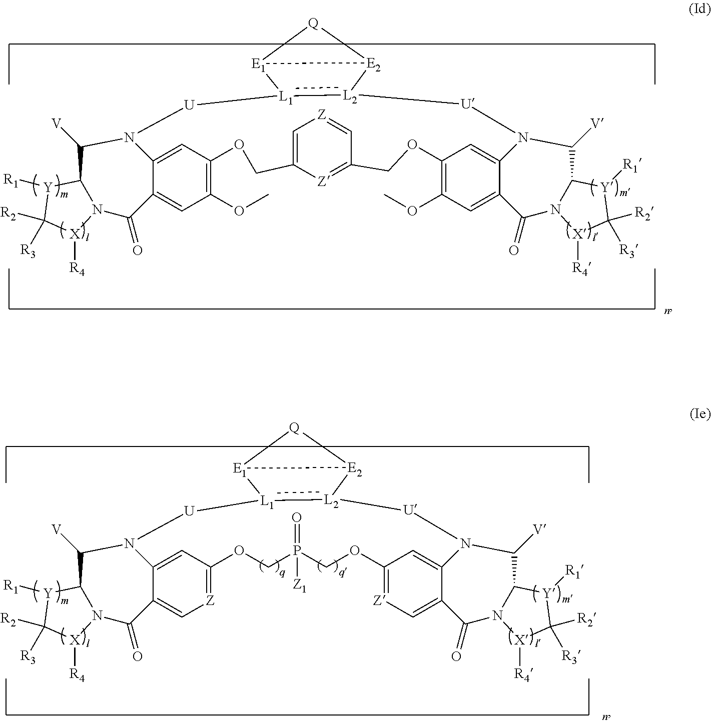
or their pharmaceutically acceptable salts, hydrates, or hydrated salts, or the polymorphic crystalline structures of these compounds or their optical isomers, racemates, diastereomers or enantiomers;
wherein Z₁ is OH, NH₂, OR₁, NHR₁, NR₁R₂, SR₁, NHR₁COX₁R₁, OR₁COX₁R₁, or N(R₂)R₁COX₁R₁; [symbol] , -----, X, X′, Y, Y′, Z, Z′, l, l′, m, m′, n, q, q′, R₁, R₁′, R₂, R₂′, R₃, R₃′, R₄, R₄′, V, V′, U, U′ L₁, L₂, E₁, E₂ and Q are defined the same as in claim 1.

3. The cross-linked PBD dimer derivative or its conjugate to a cell-binding molecule according to claim 1 having one of the Formula (I-01)-(I-19) below: 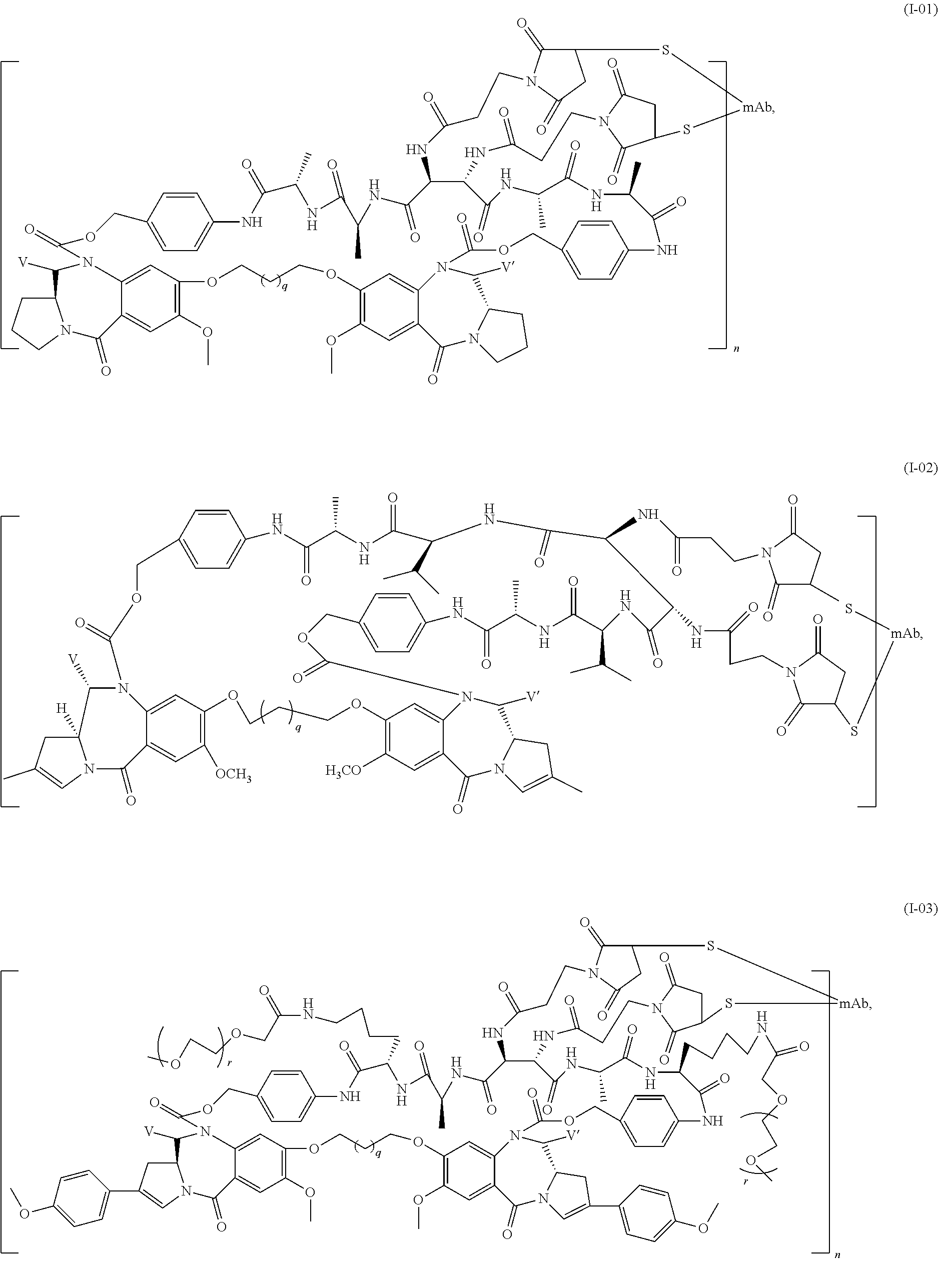 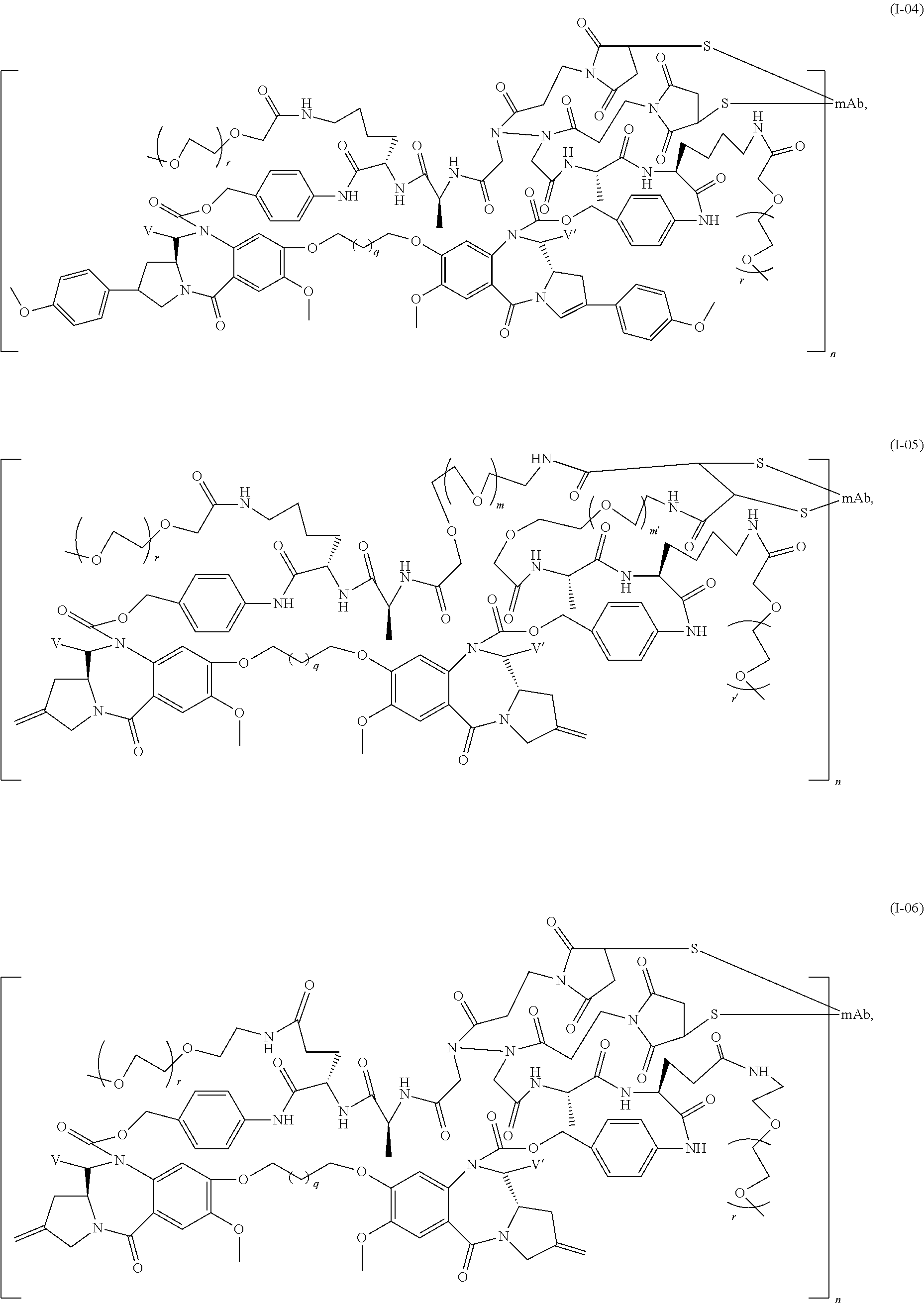 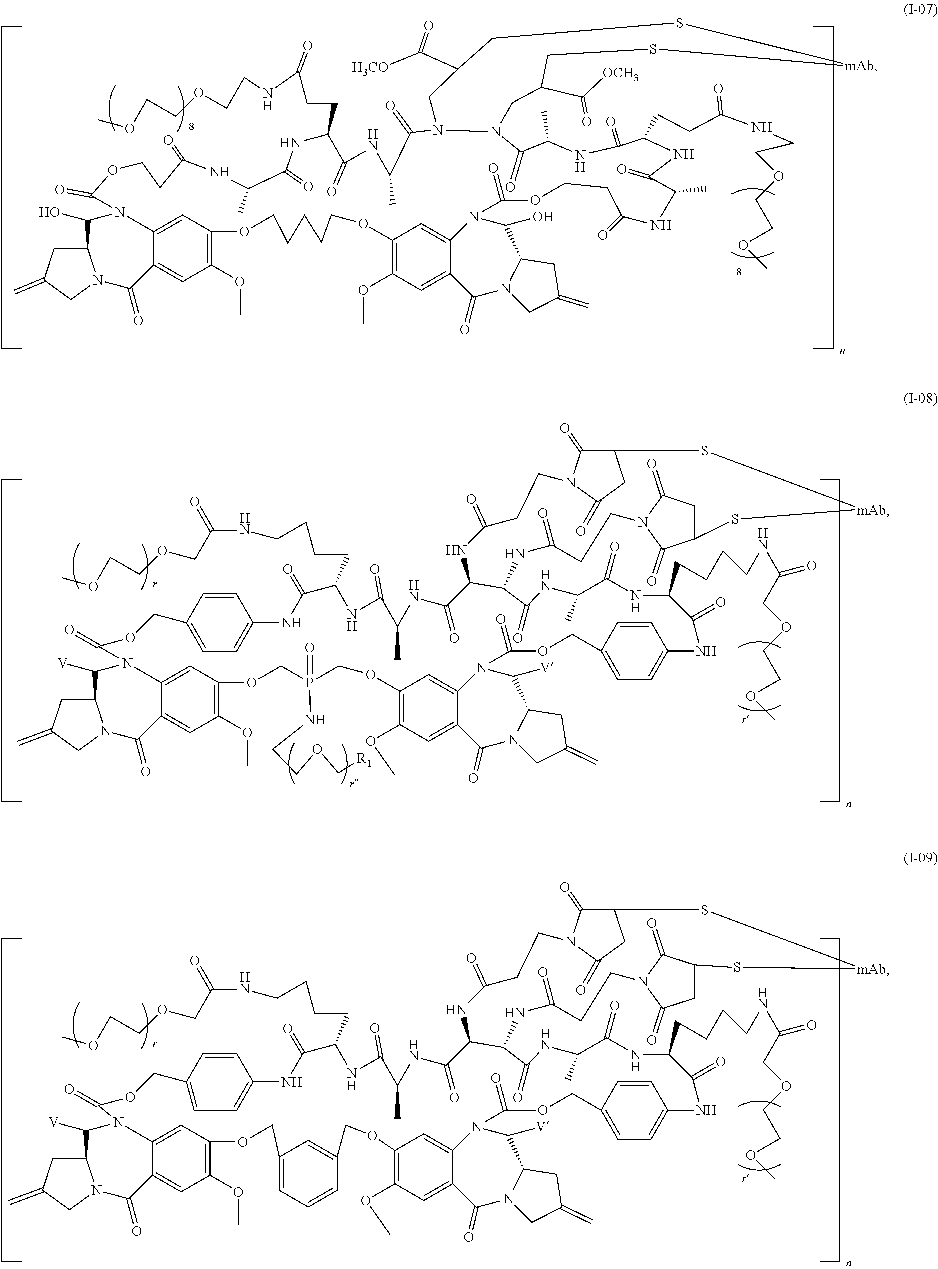 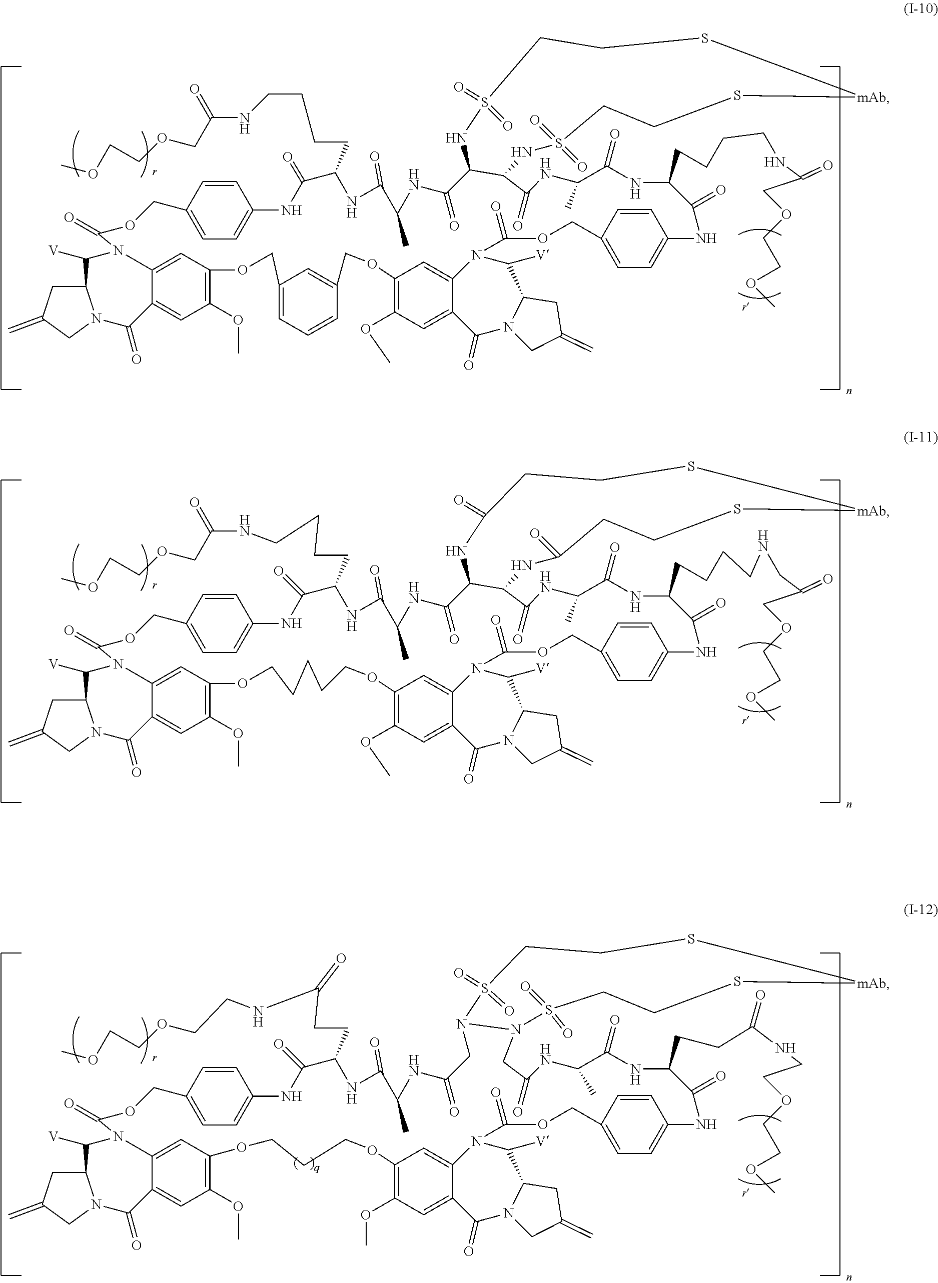 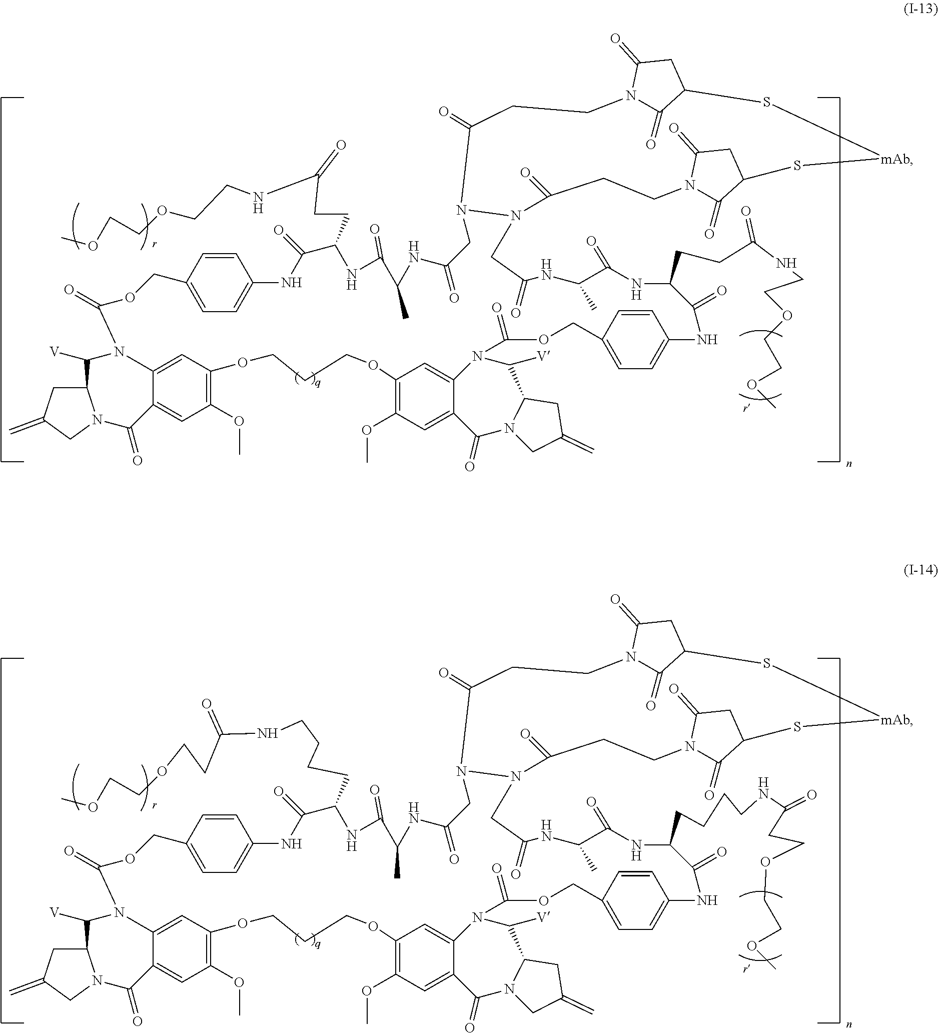 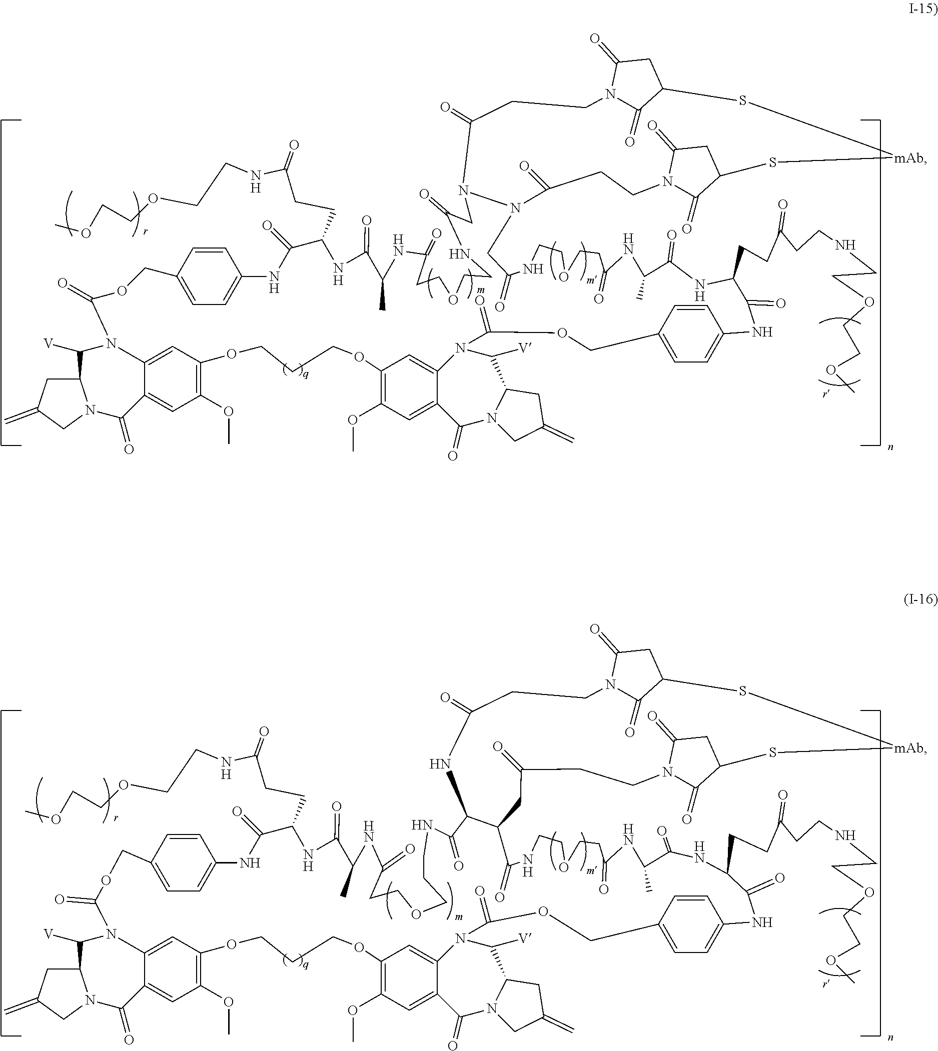 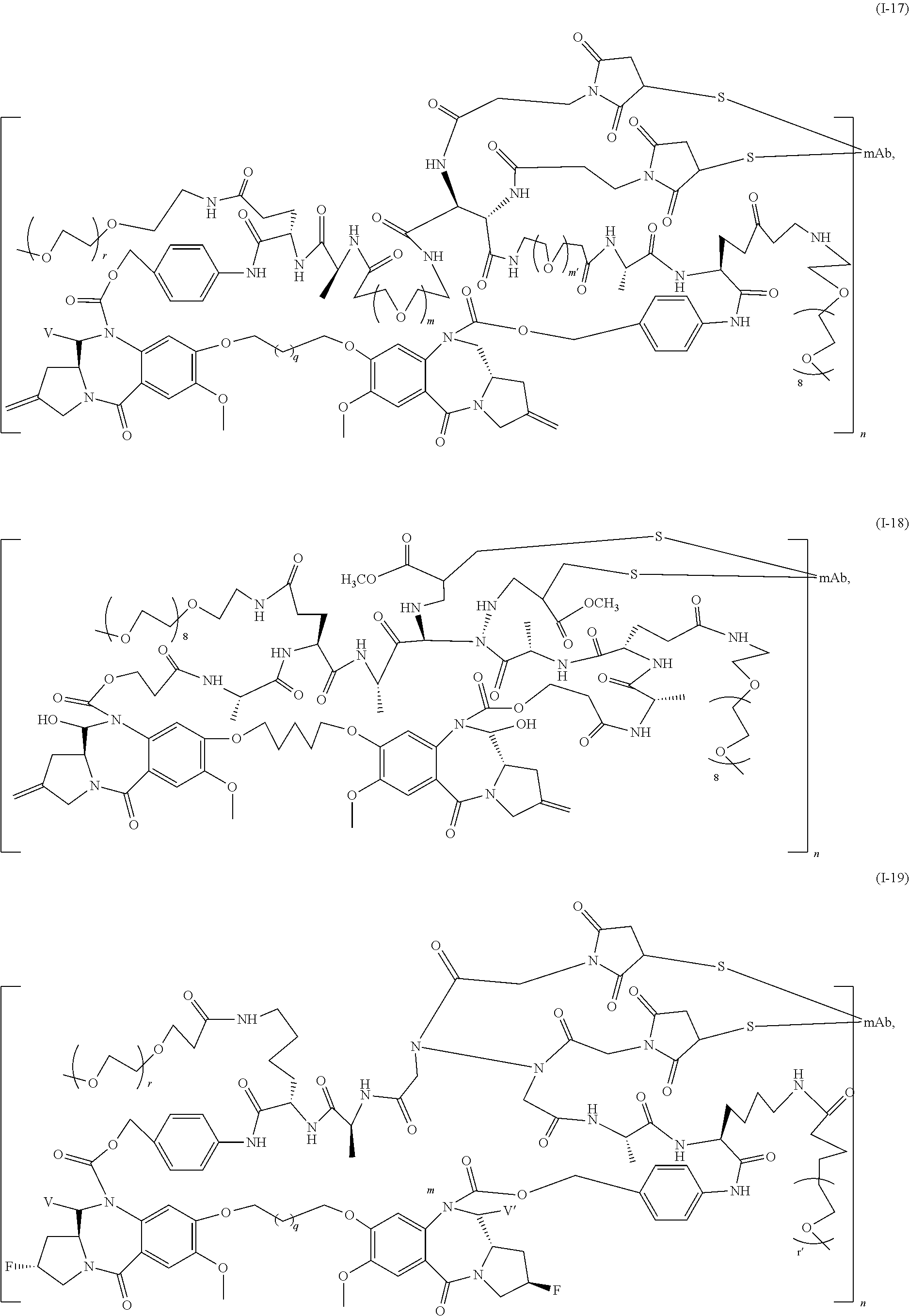 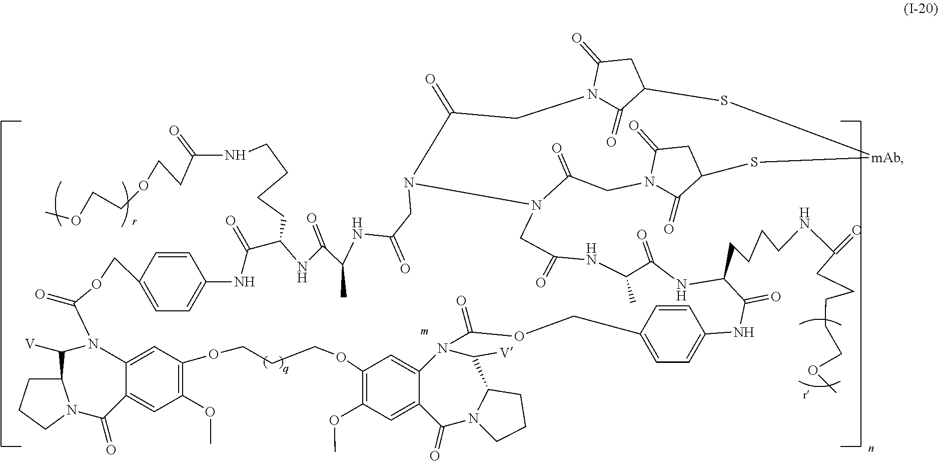
wherein V, V′, n and q are defined the same in claim 1; mAb is a cell-binding molecule; r, r′ and r″ are independently 0-200.

4. The cross-linked PBD dimer derivative or its conjugate to a cell-binding molecule having the structure of Formula (II), (III) or (IV): 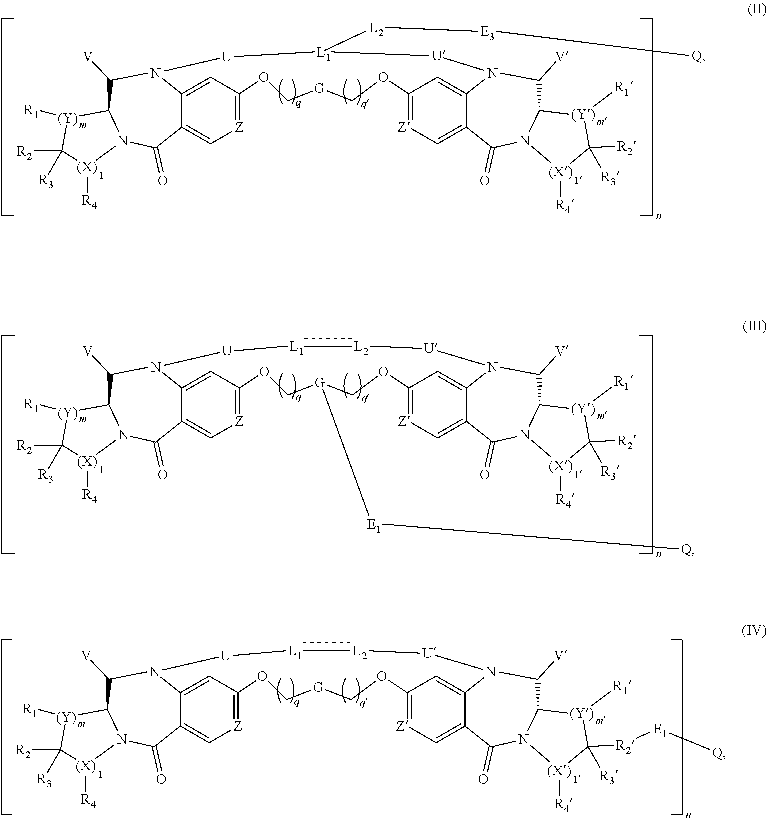
wherein [symbol] , -----, X, X′, Y, Y′, Z, Z′, l, l′, m, m′, n, q, q′, R₁, R₁′, R₂, R₂′, R₃, R₃′, R₄, R₄′, V, V′, U, U′ L₁, L₂, G, Q, E, and E₂ are the same defined as in claim 1.

5. The cross-linked PBD dimer derivative or its conjugate to a cell-binding molecule according to claim 4 having one of the Formula (II-01)-(II-08), (III-01)-(III-05) and (IV-01)-(IV-11) below: 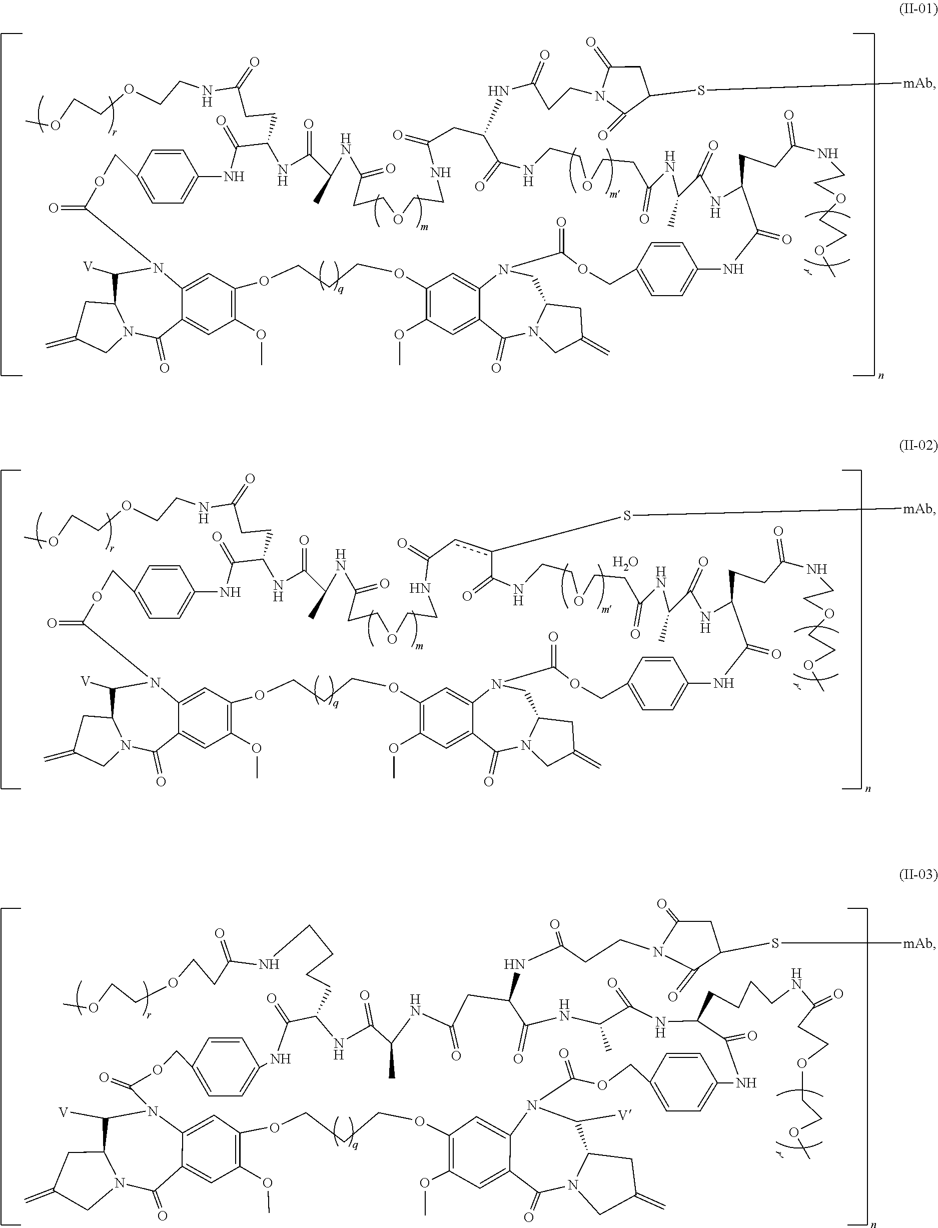 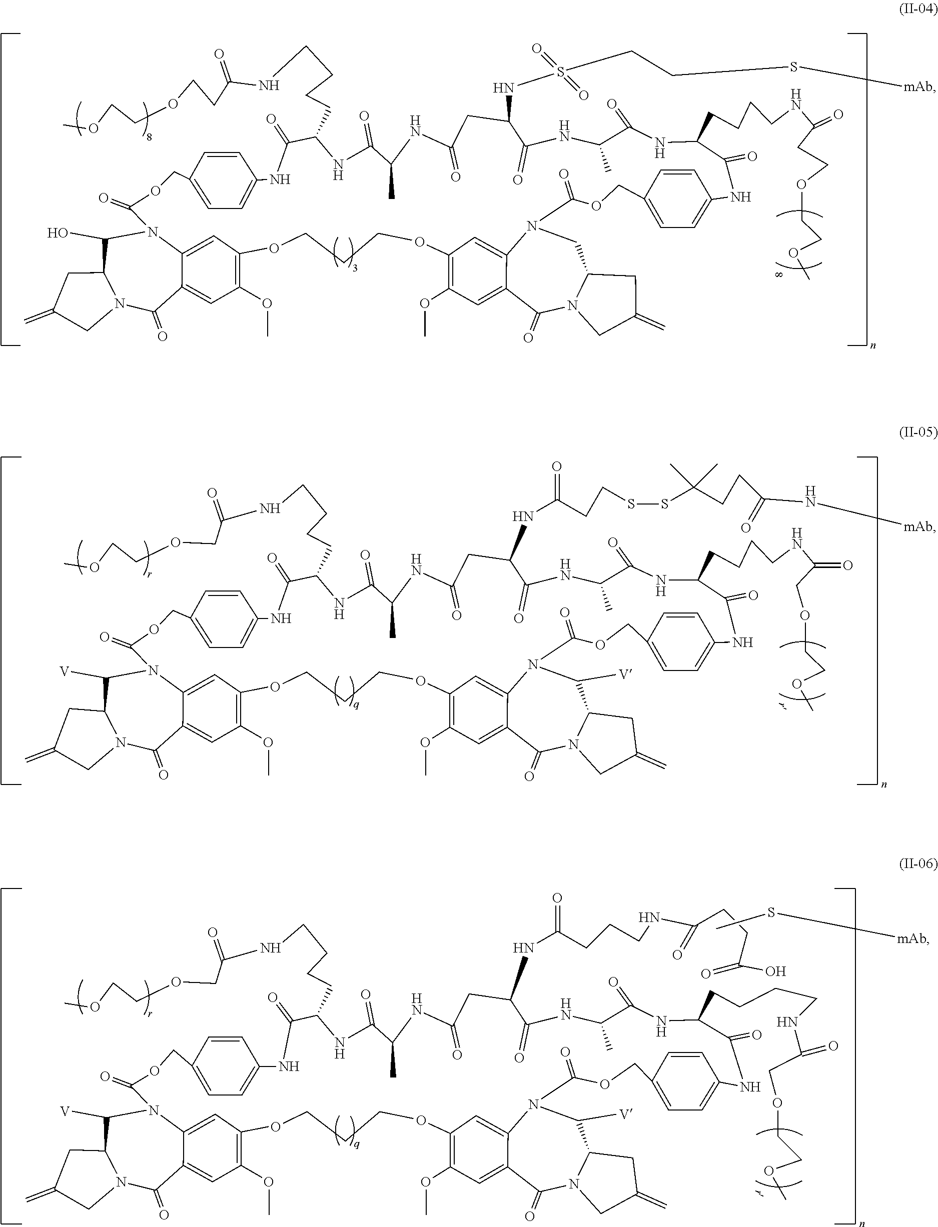 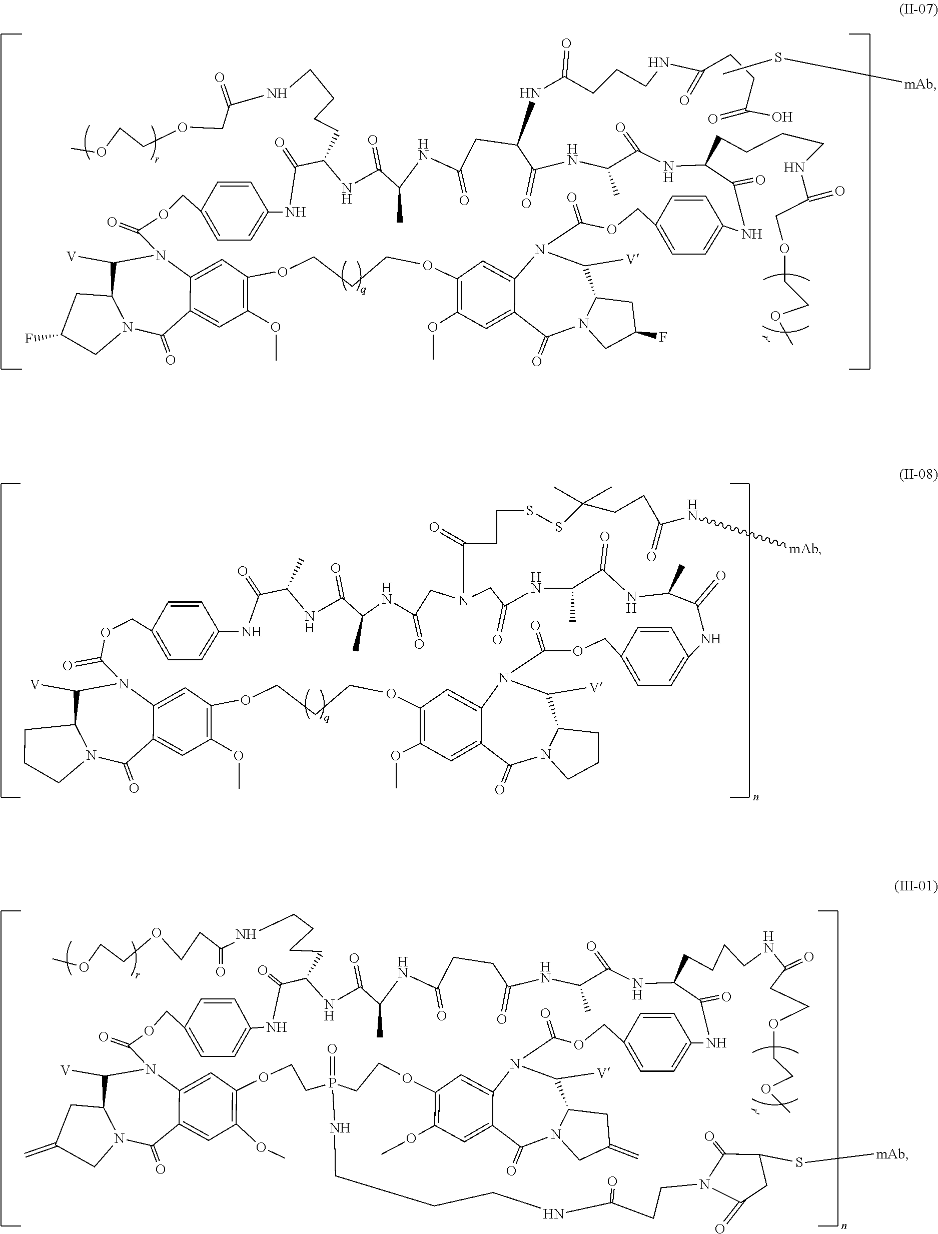 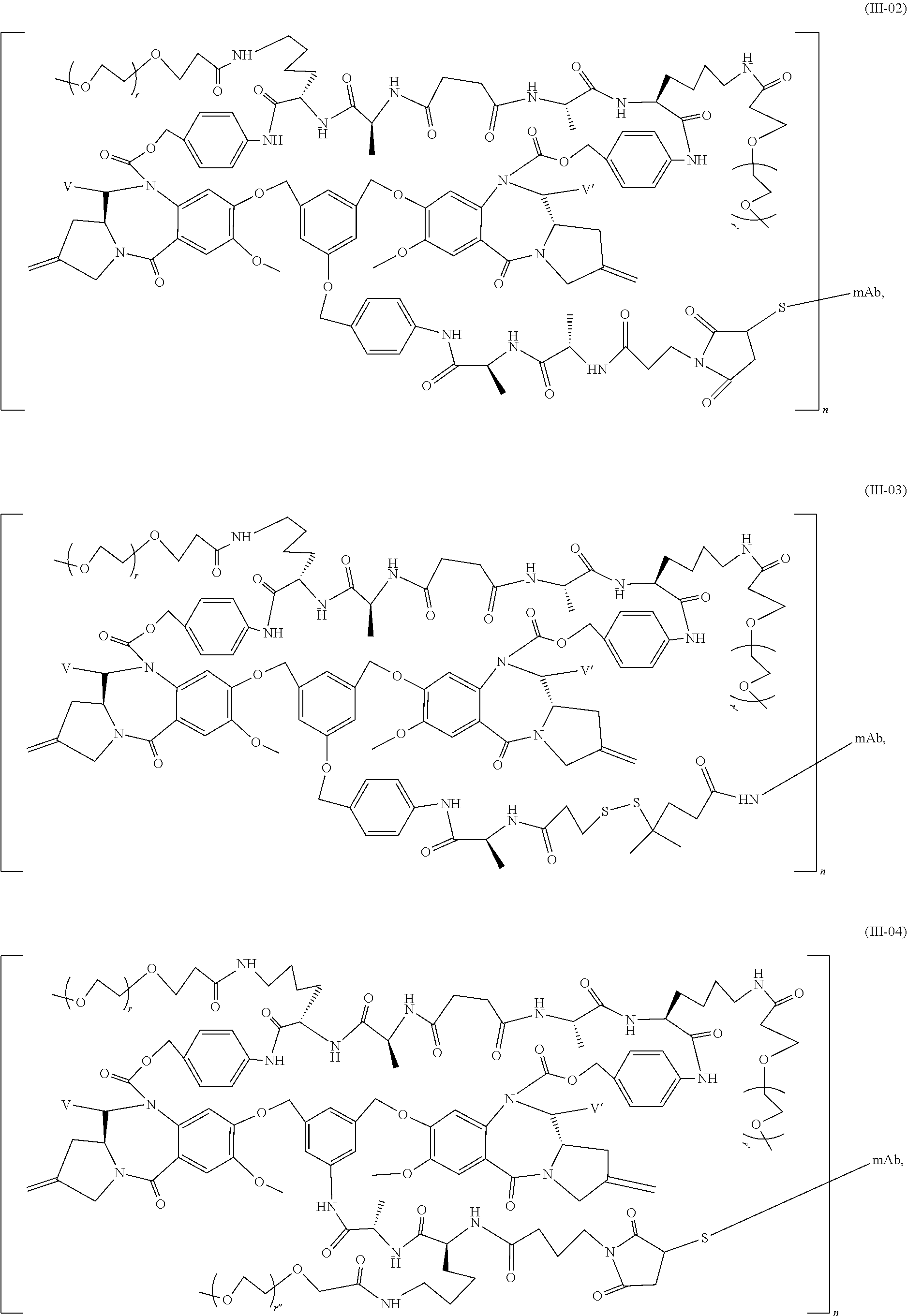 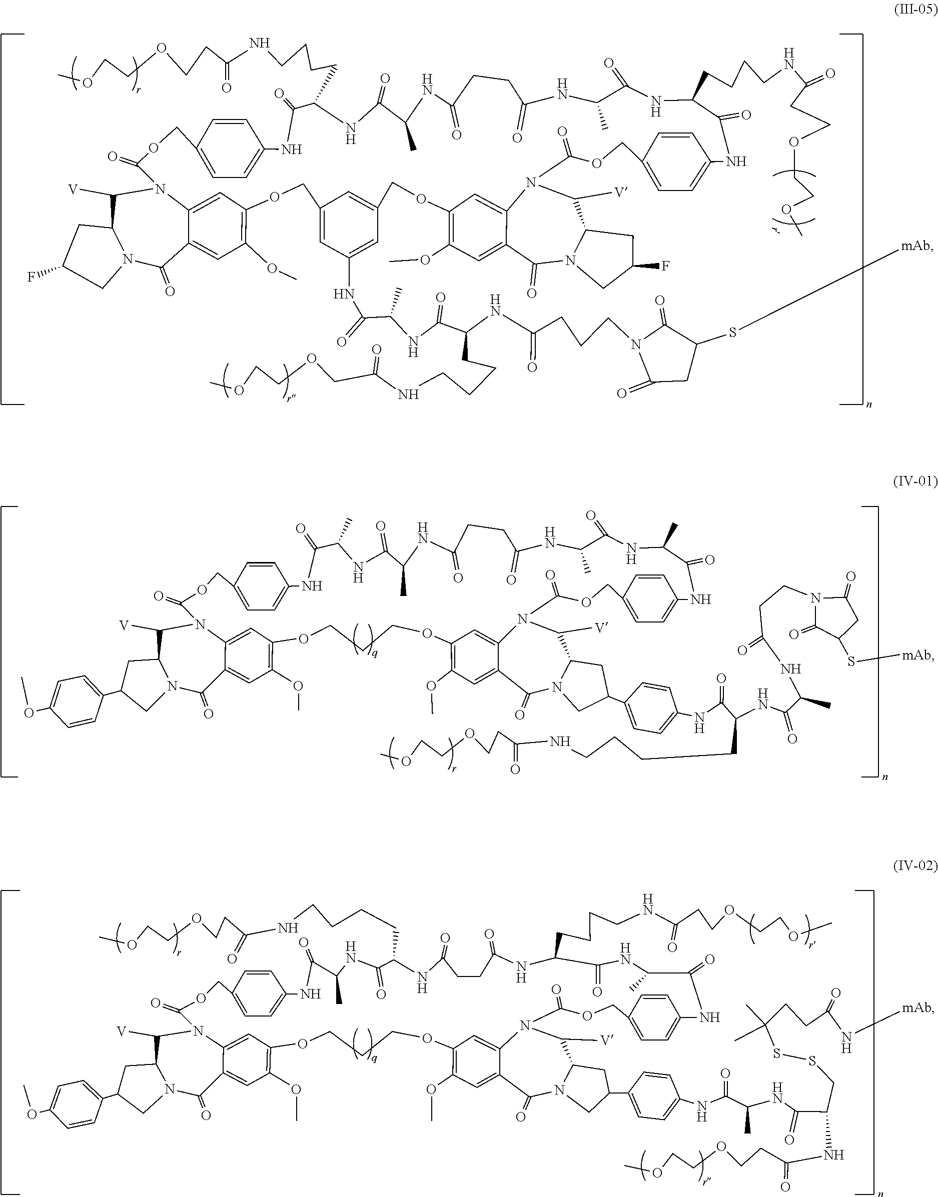 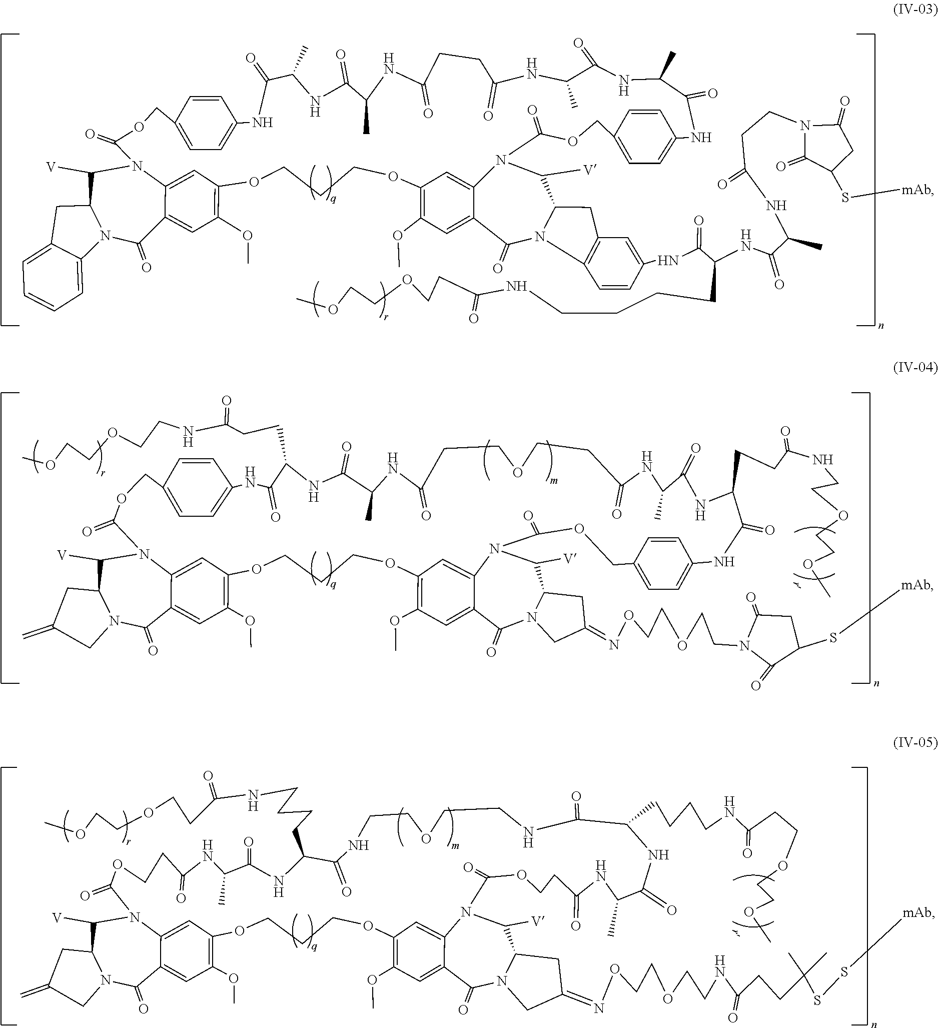 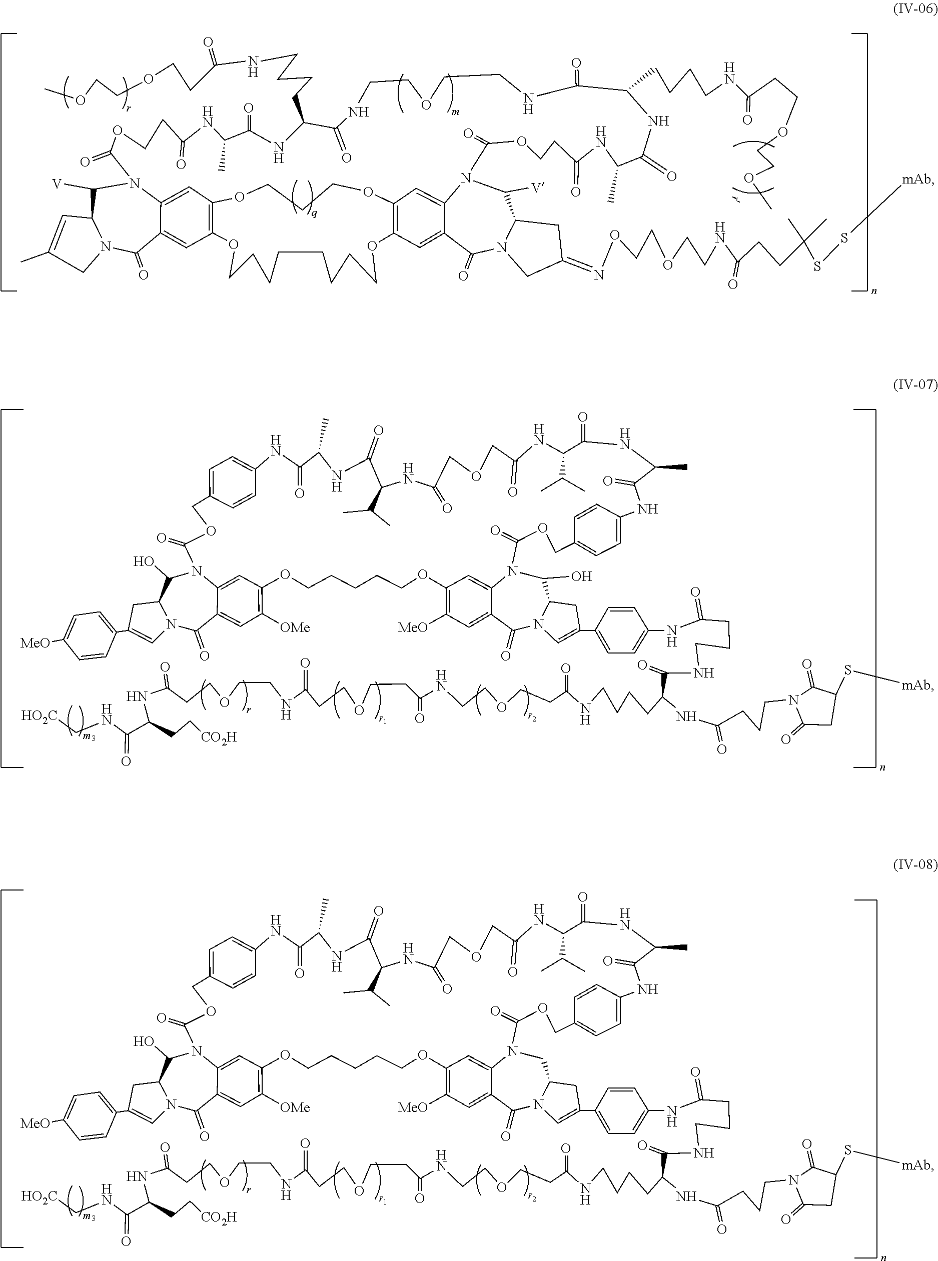 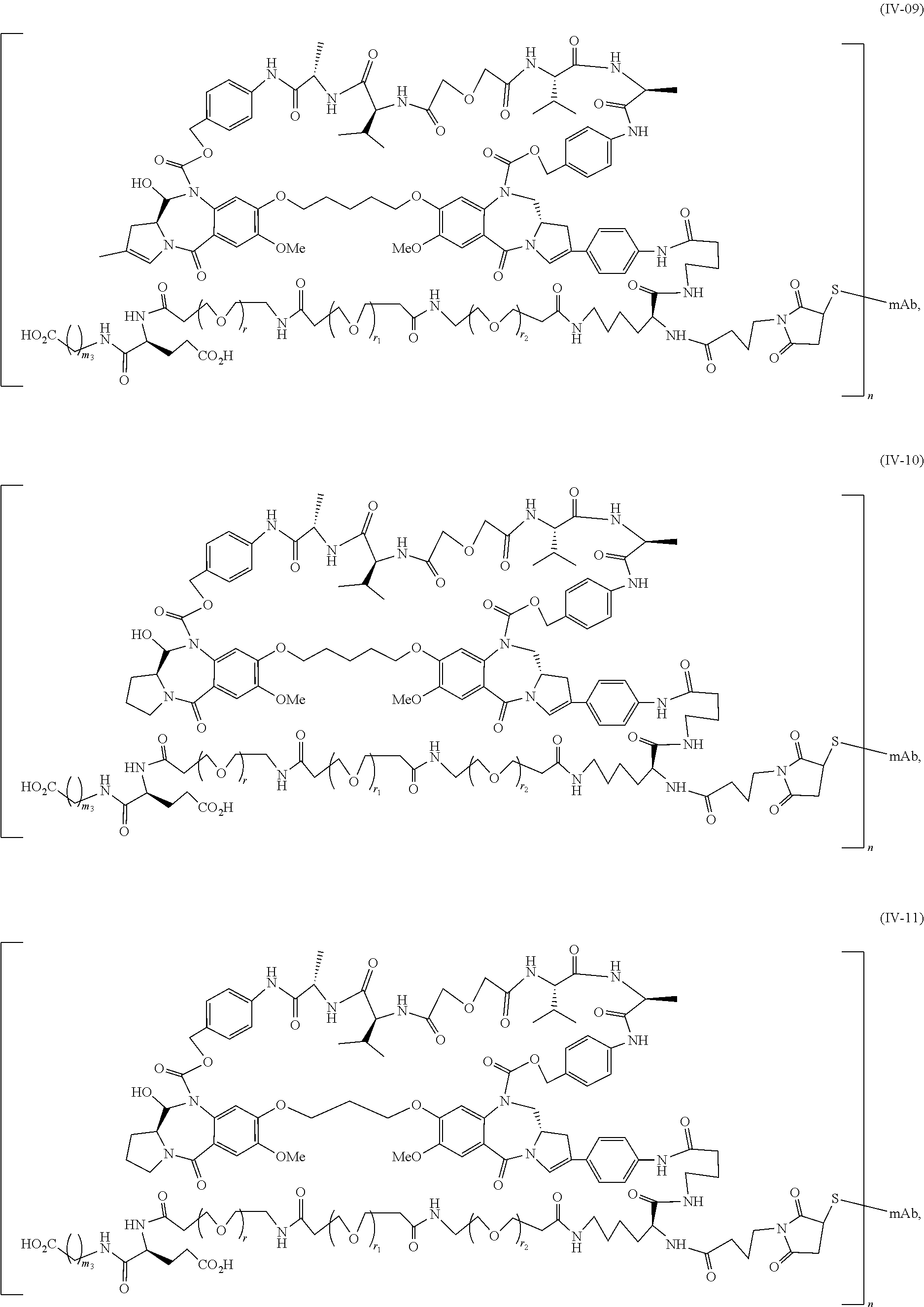
wherein [symbol] , -----, m, m′, n, q, and q′ are the same defined as in claim 4; r, r′, and r″ are independently 0-200, m₃ is 0-30.

6. A cross-linked PBD dimer derivative or its conjugate to a cell-binding molecule having the Formula (V) (VI), (VII), or (VIII): 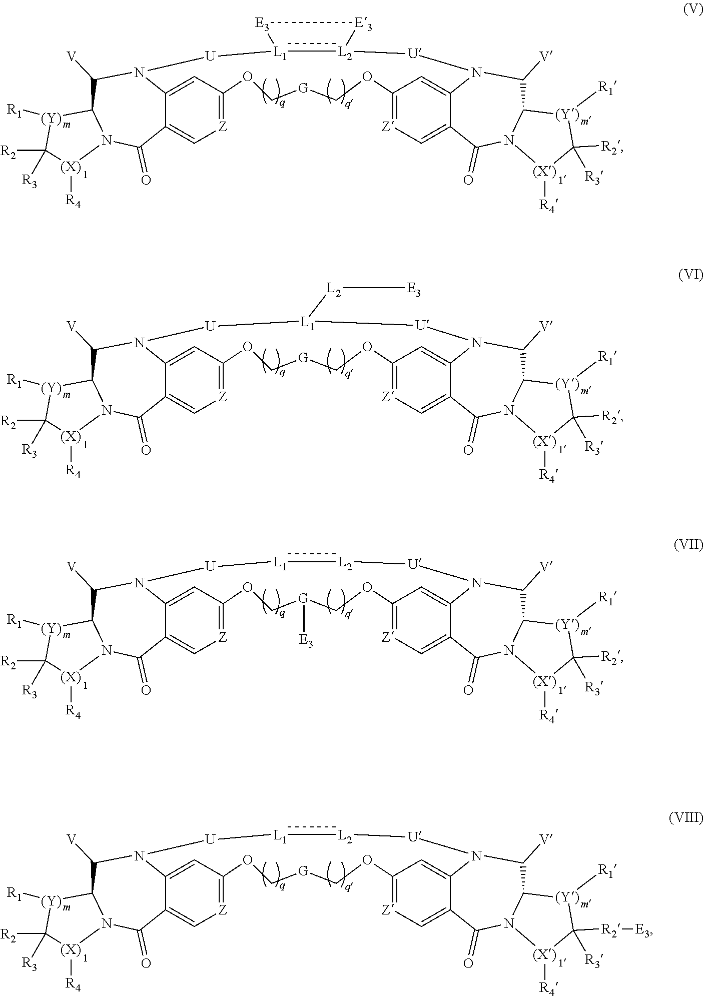
wherein [symbol] , -----, X, X′, Y, Y′, Z, Z′, l, l′, m, m′, n, q, q′, R₁, R₁′, R₂, R₂′, R₃, R₃′, R₄, R₄′, V, V′, U, U′ L₁, L₂, E₁, and E₂ are the same defined as in claim 1;
wherein E₃ and E′₃ are independently selected from: 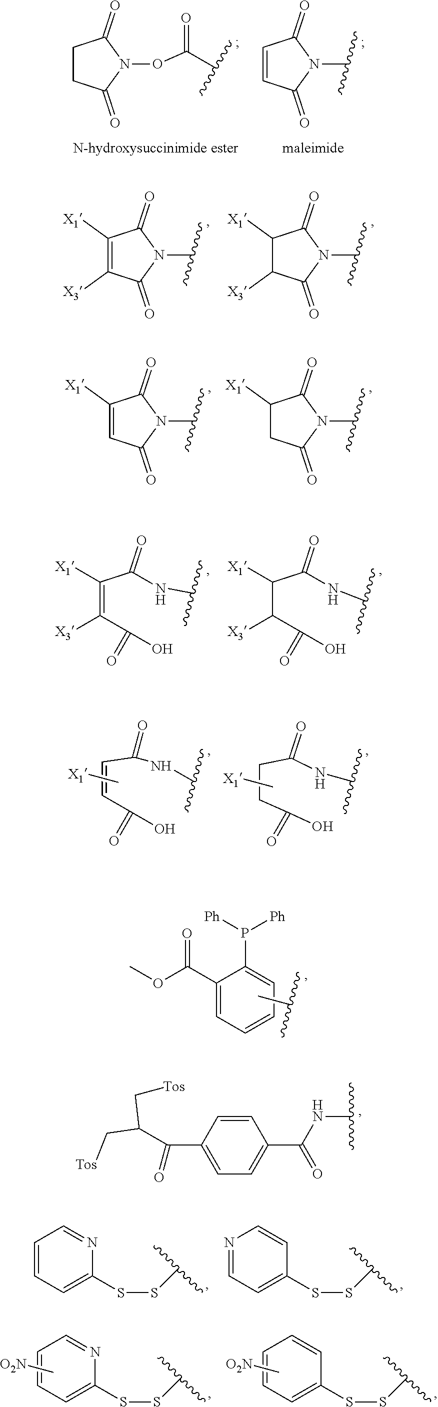 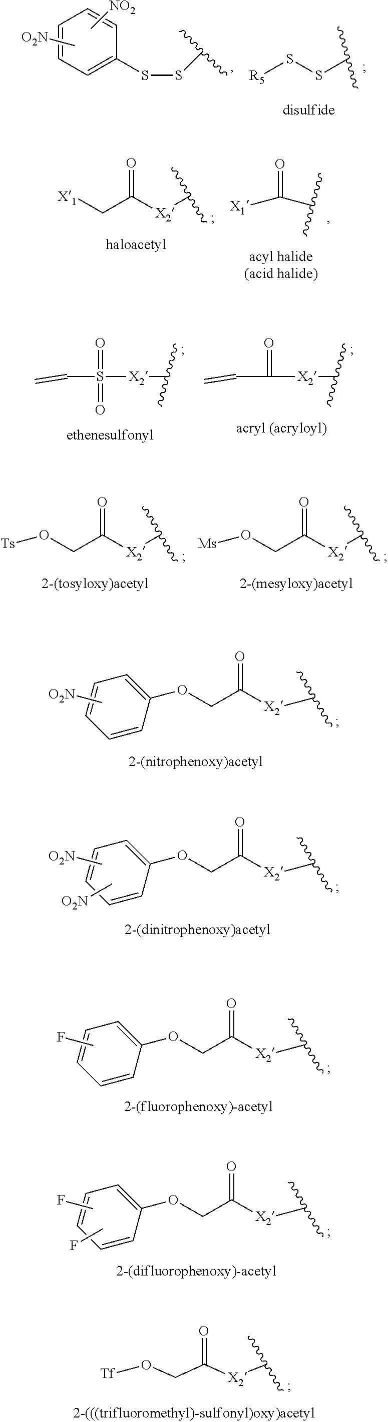 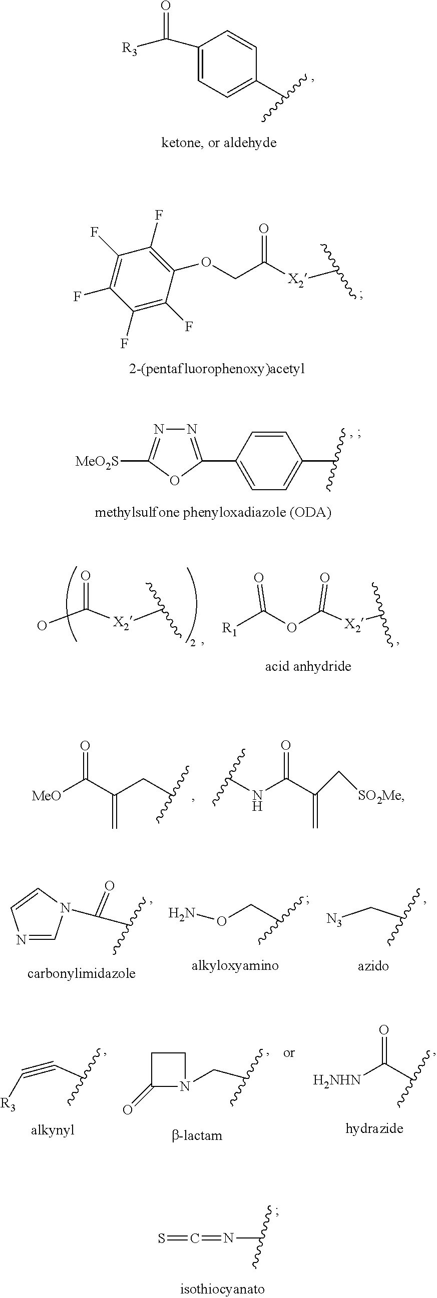
wherein X₁′ and X₃′ are independently F, Cl, Br, I or Lv₃; X₂′ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from methanesulfonyl (mesyl), toluenesulfonyl (tosyl), trifluoromethyl-sulfonyl (triflate), trifluoromethylsulfonate, nitrophenoxyl, phenylthio, pyridinylthio, N-succinimidyloxyl (NHS), phenoxyl; dinitrophenoxyl; pentafluorophenoxyl, tetrafluoro-phenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-yl, phenyloxadiazol-yl (ODA), oxadiazol-yl, or an intermediate molecule generated with a condensation reagent for Mitsunobu reactions, wherein R₁ and R₂ are defined above;
E₃ and E′₃ are independently selected from —SH, —S—SCH₃, —S—SAc, —S—S-pyridine, —S—S—Ar(—NO₂), —S-cell-binding agent, or any one of the following formulas: 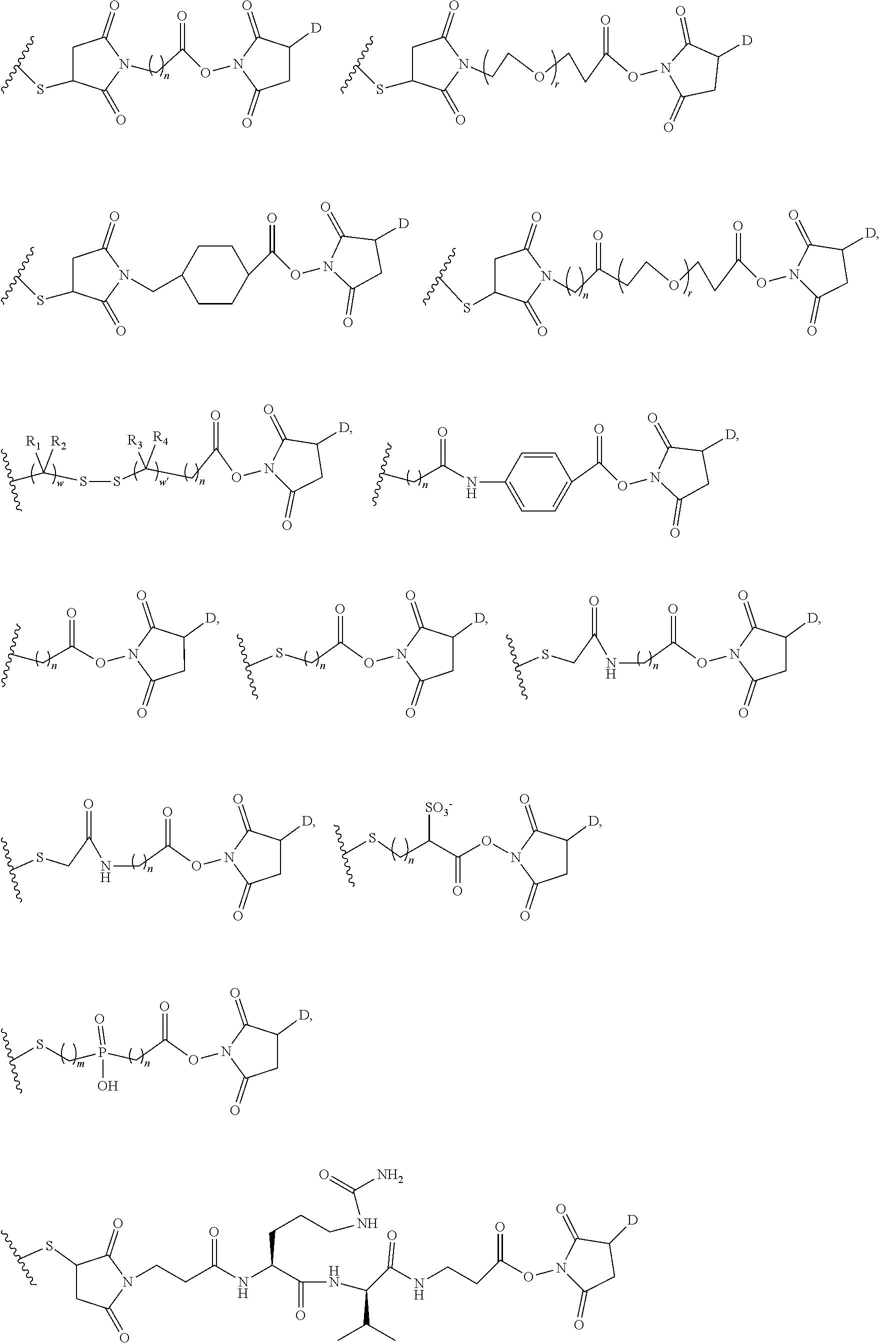
wherein D is H, —NO₂, SO³H, or F; wherein R₁, R₂, R₃, R₄, r, m and n are defined above; w and w′ are independently 0, 1 or 2;
wherein R₅ and R₅′ are independently selected from C₁-C₆ alkyl, aryl, cyclic, cyclohetero, H, or M, wherein M is Na, K, Ca, ammonium or the other pharmaceutically acceptable salt.

7. The cross-linked PBD dimer derivative or its conjugate to a cell-binding molecule according to claim 6 having one of Formulas (V-01)-(V-20), (VI-01)-(VI-12), (VII-01)-(VII-06), (VIII-01)-(VIII-06) below: 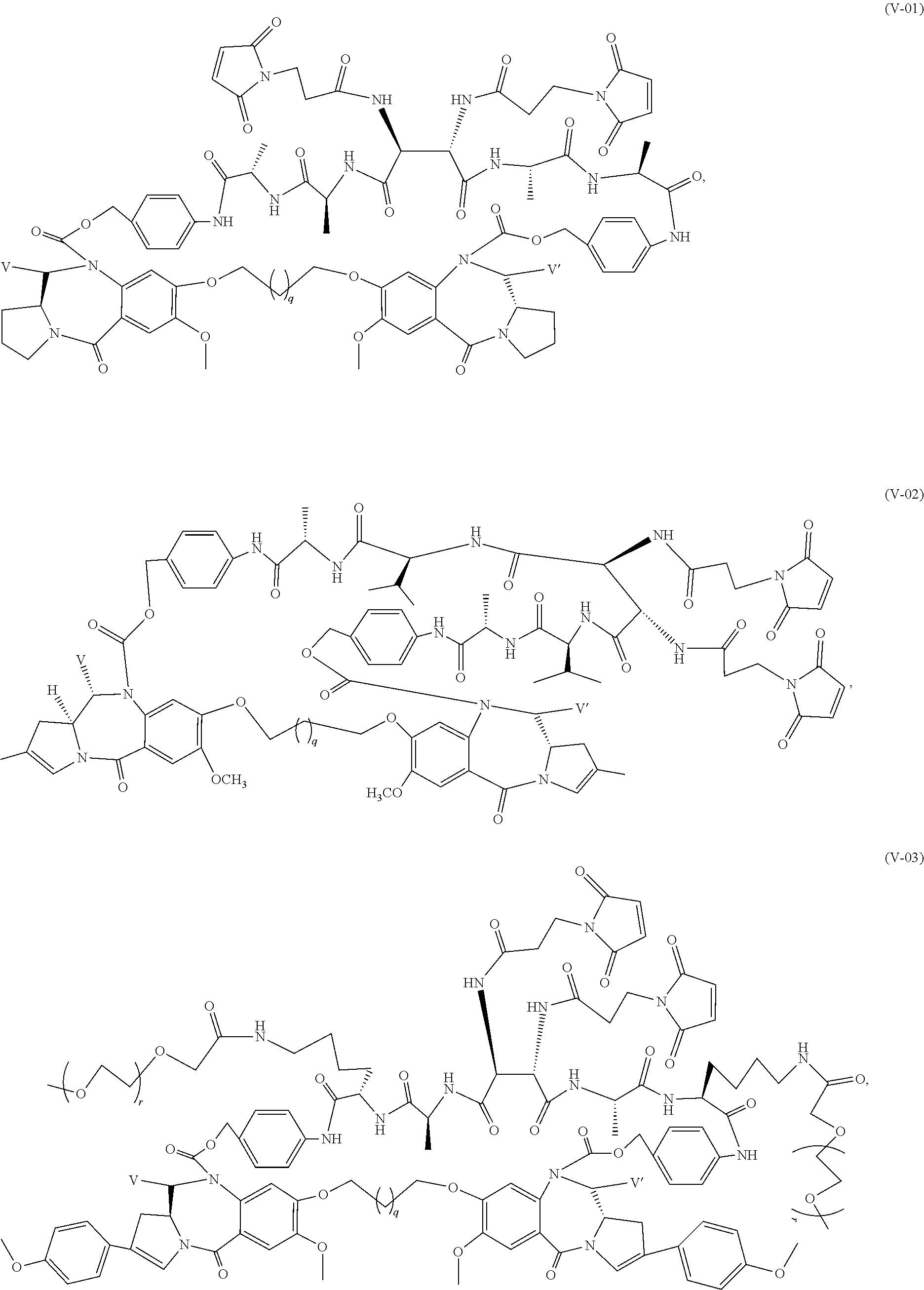 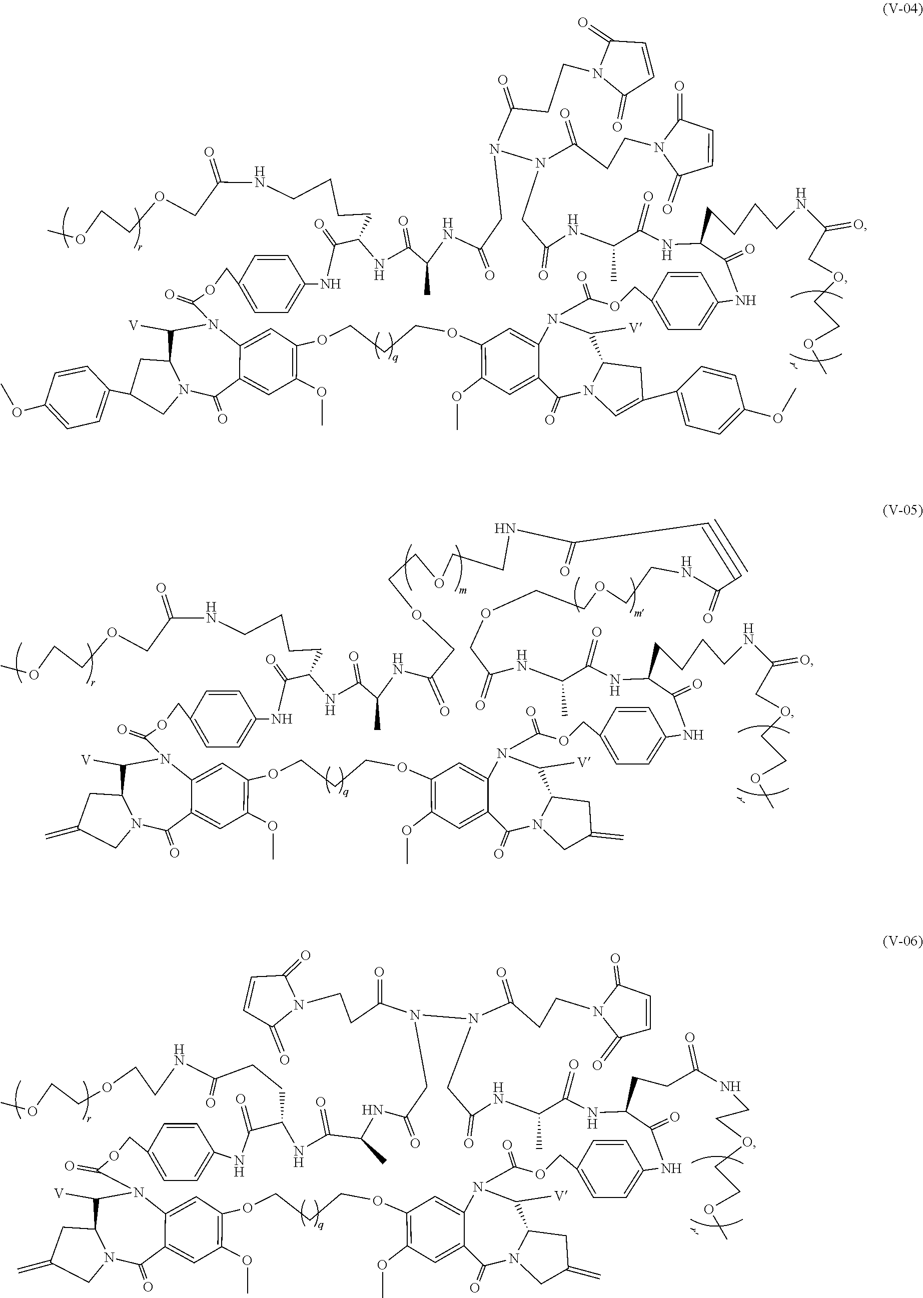 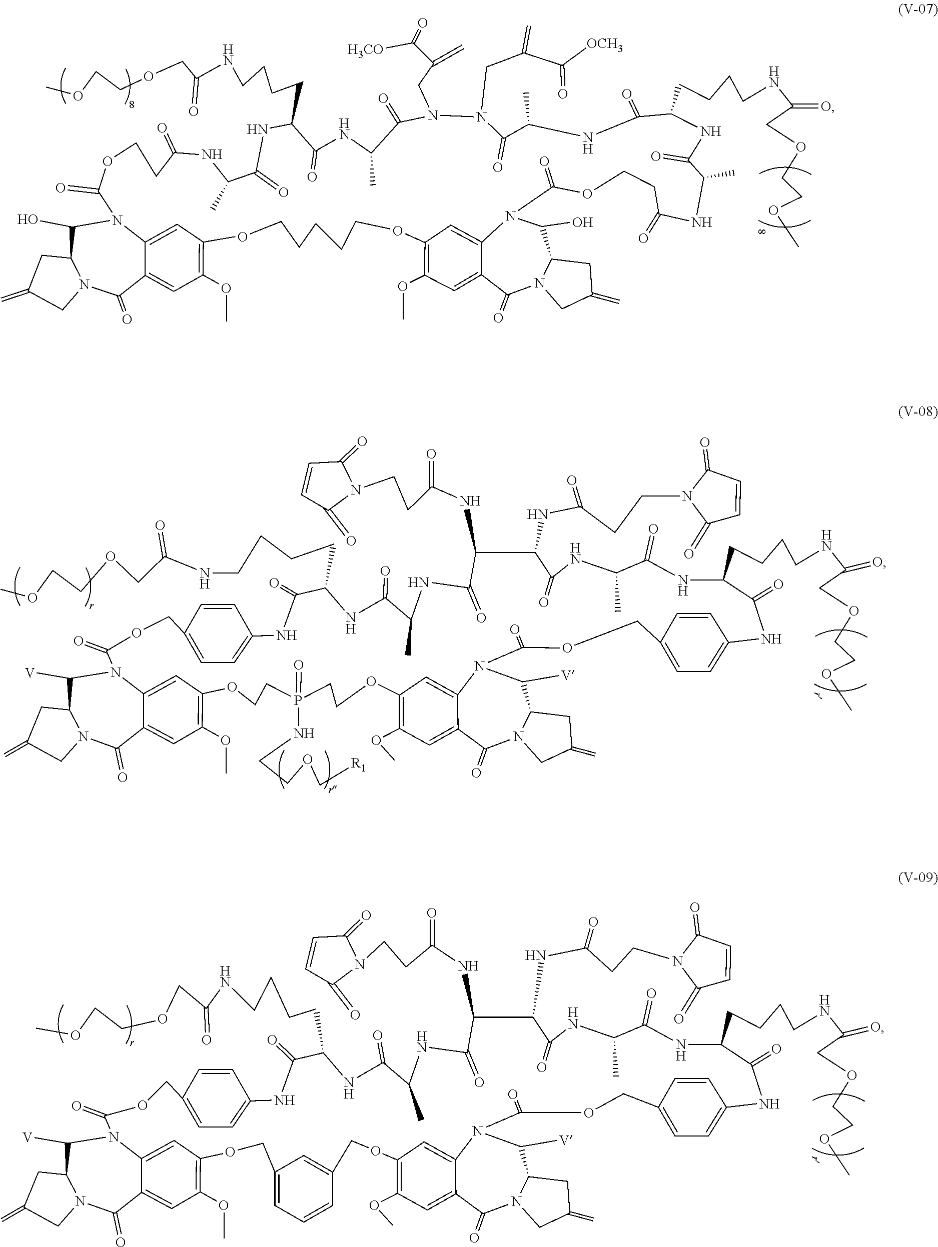 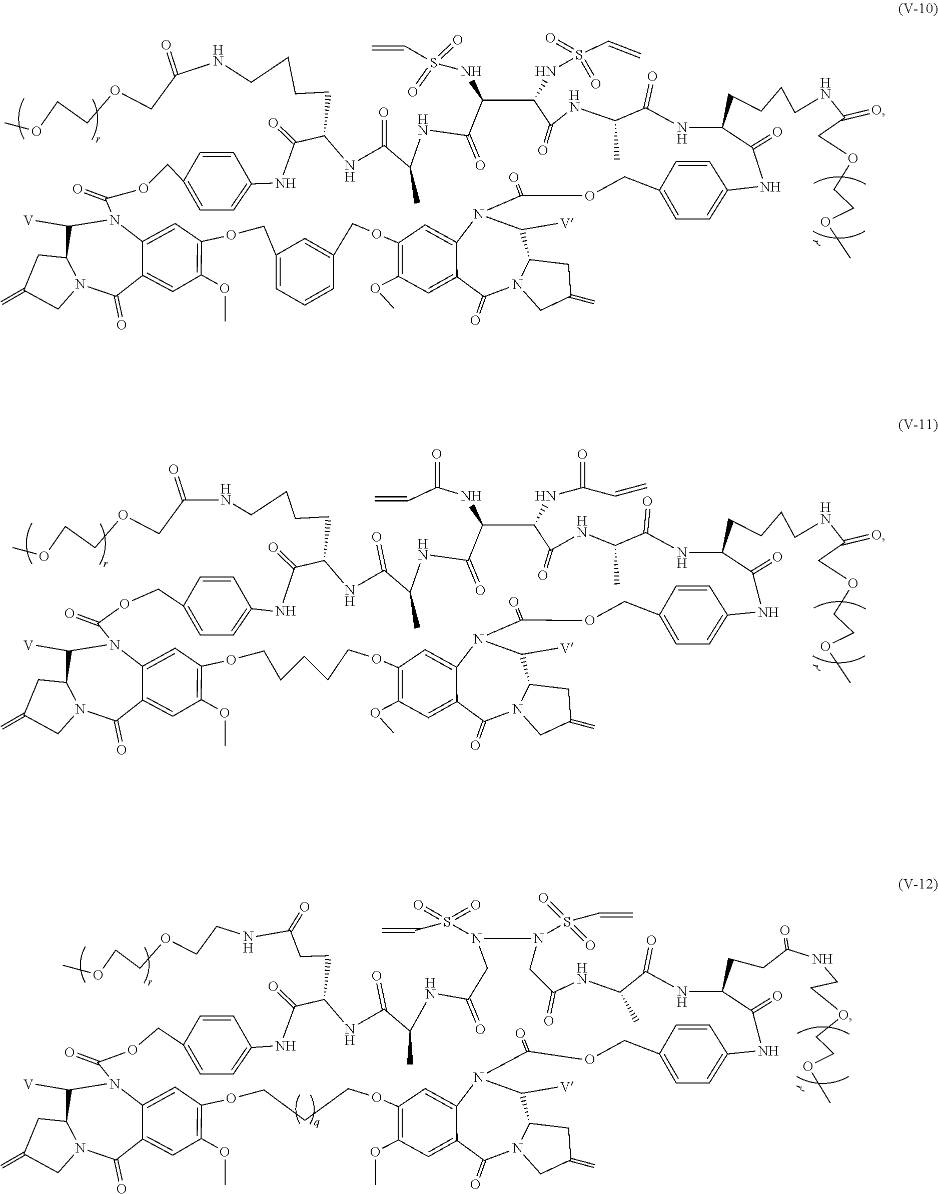 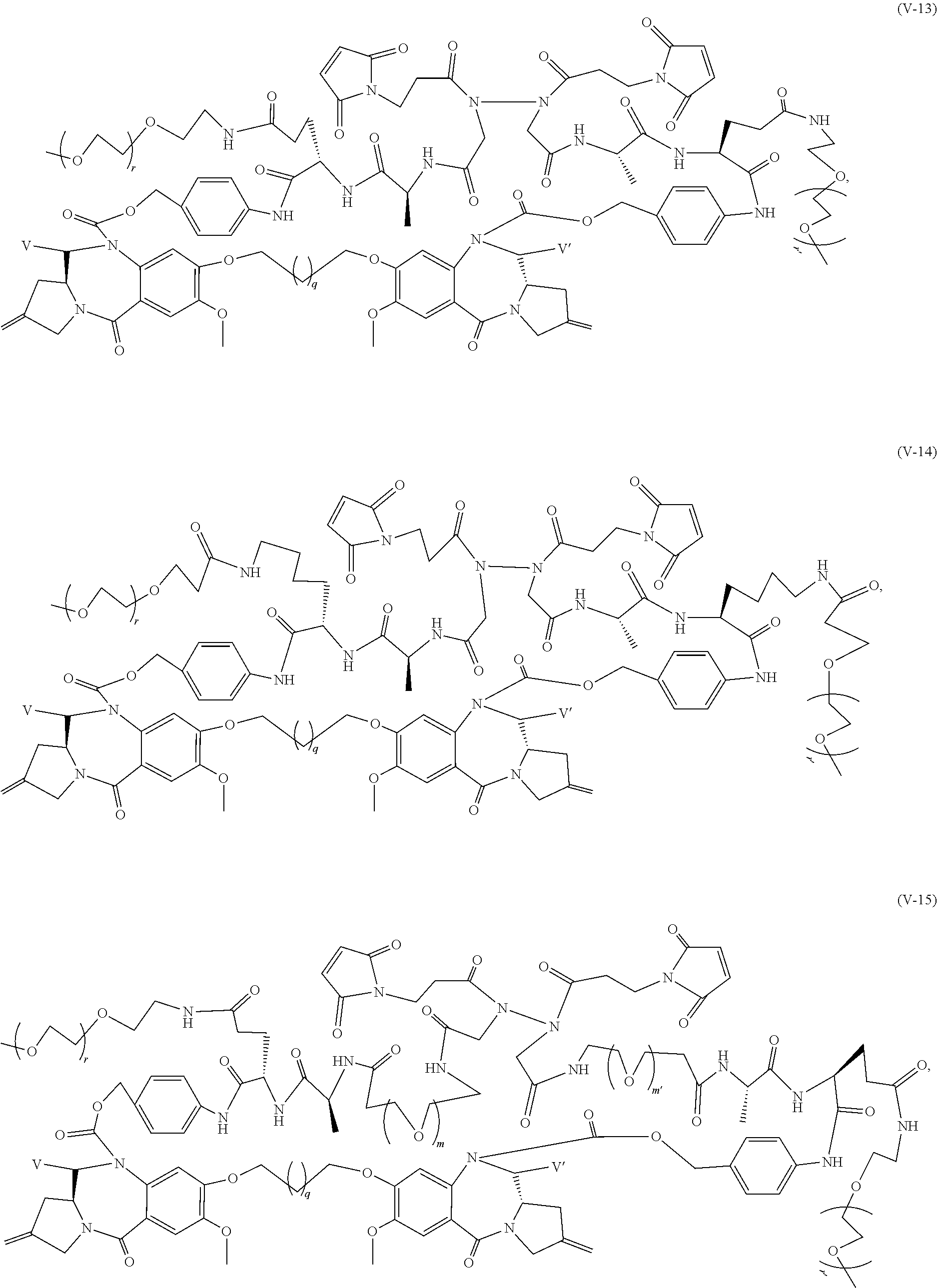 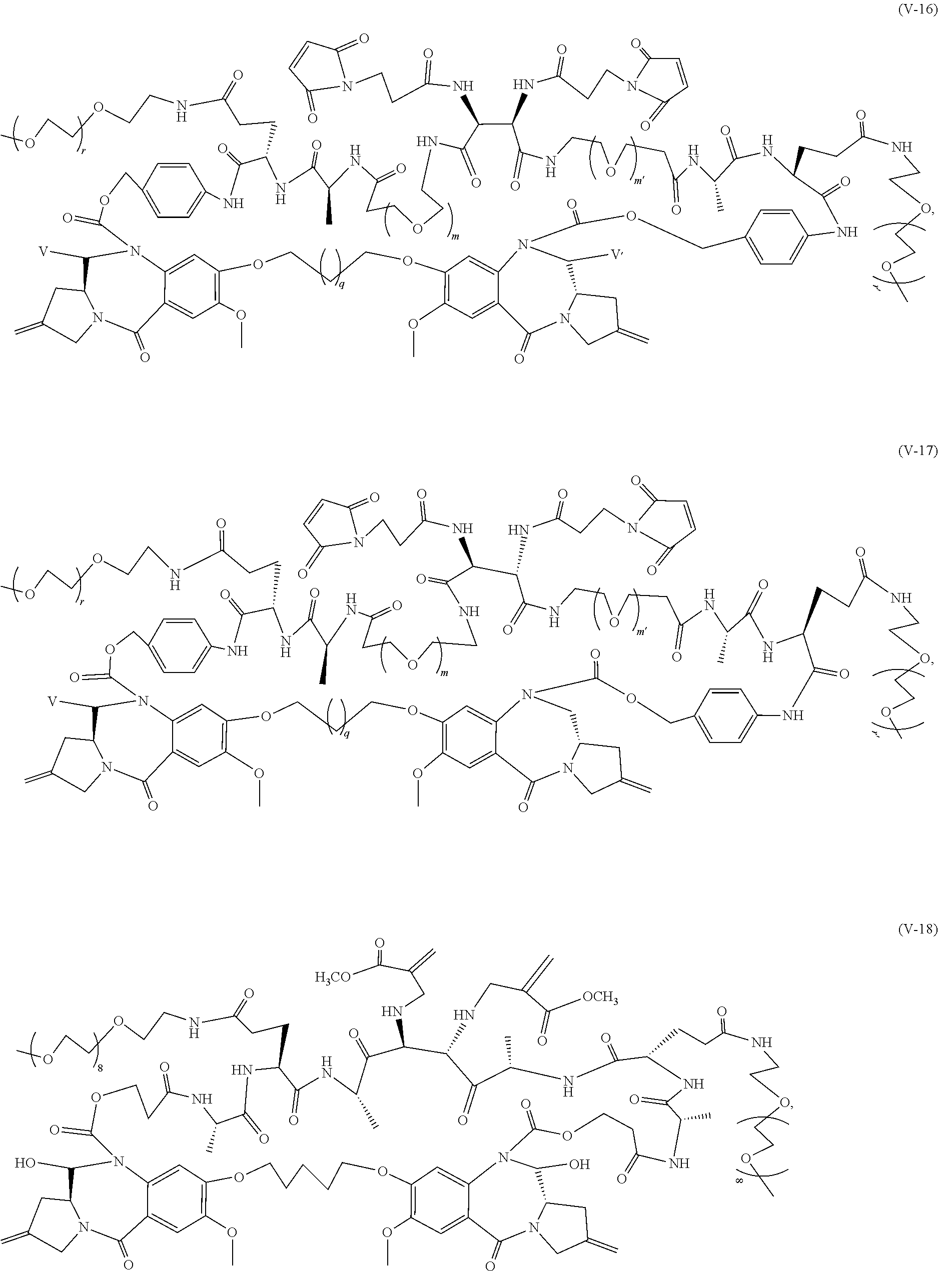 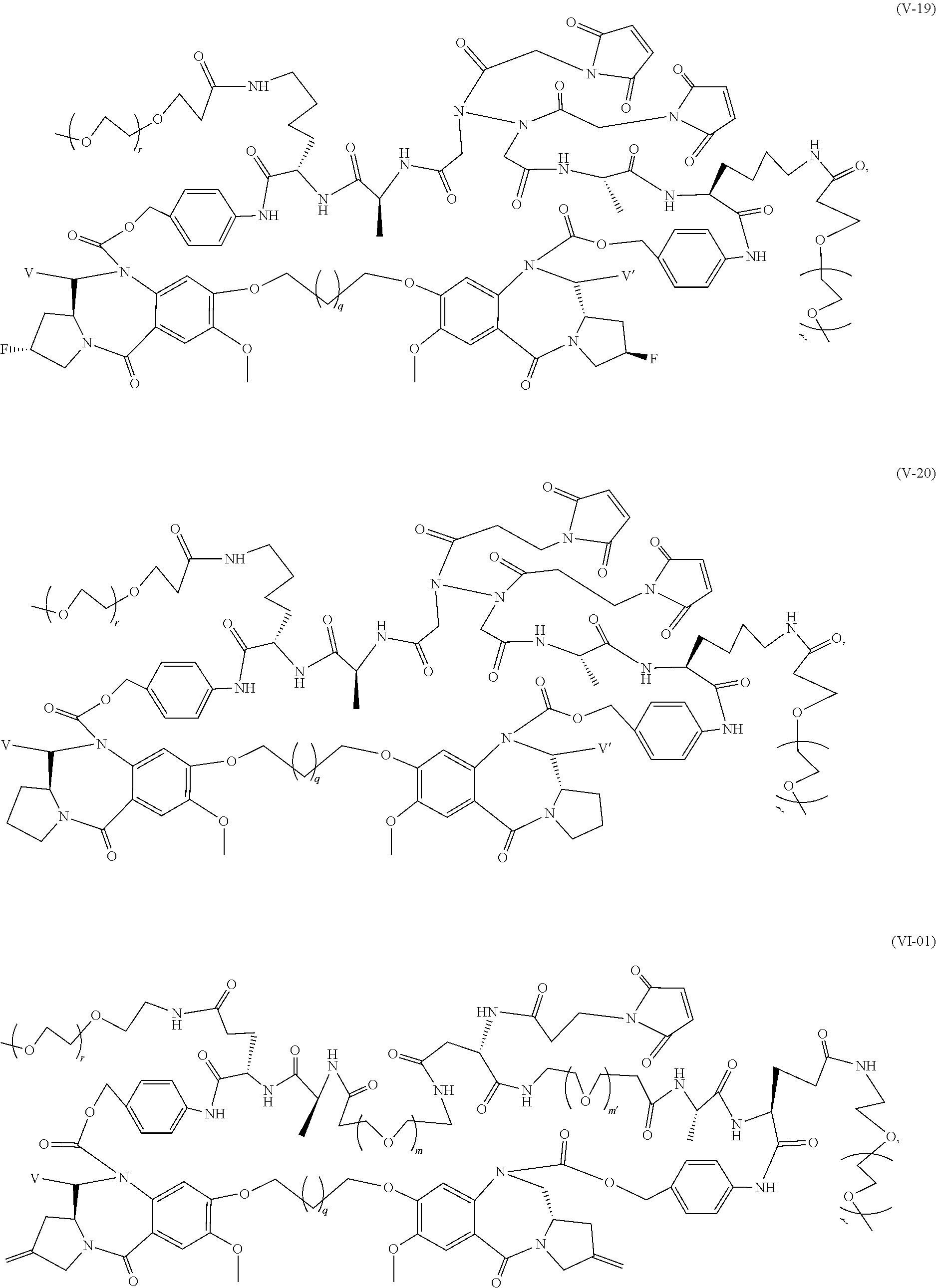 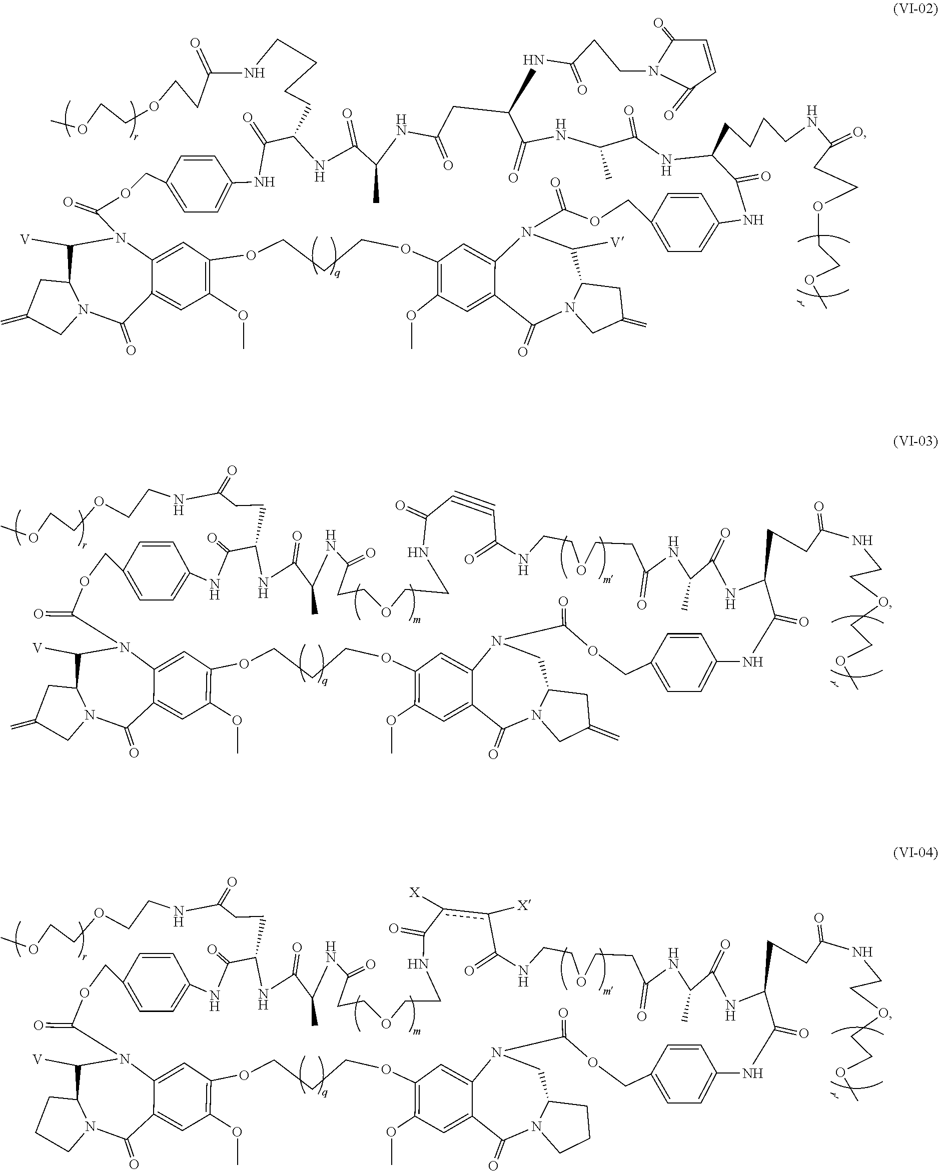 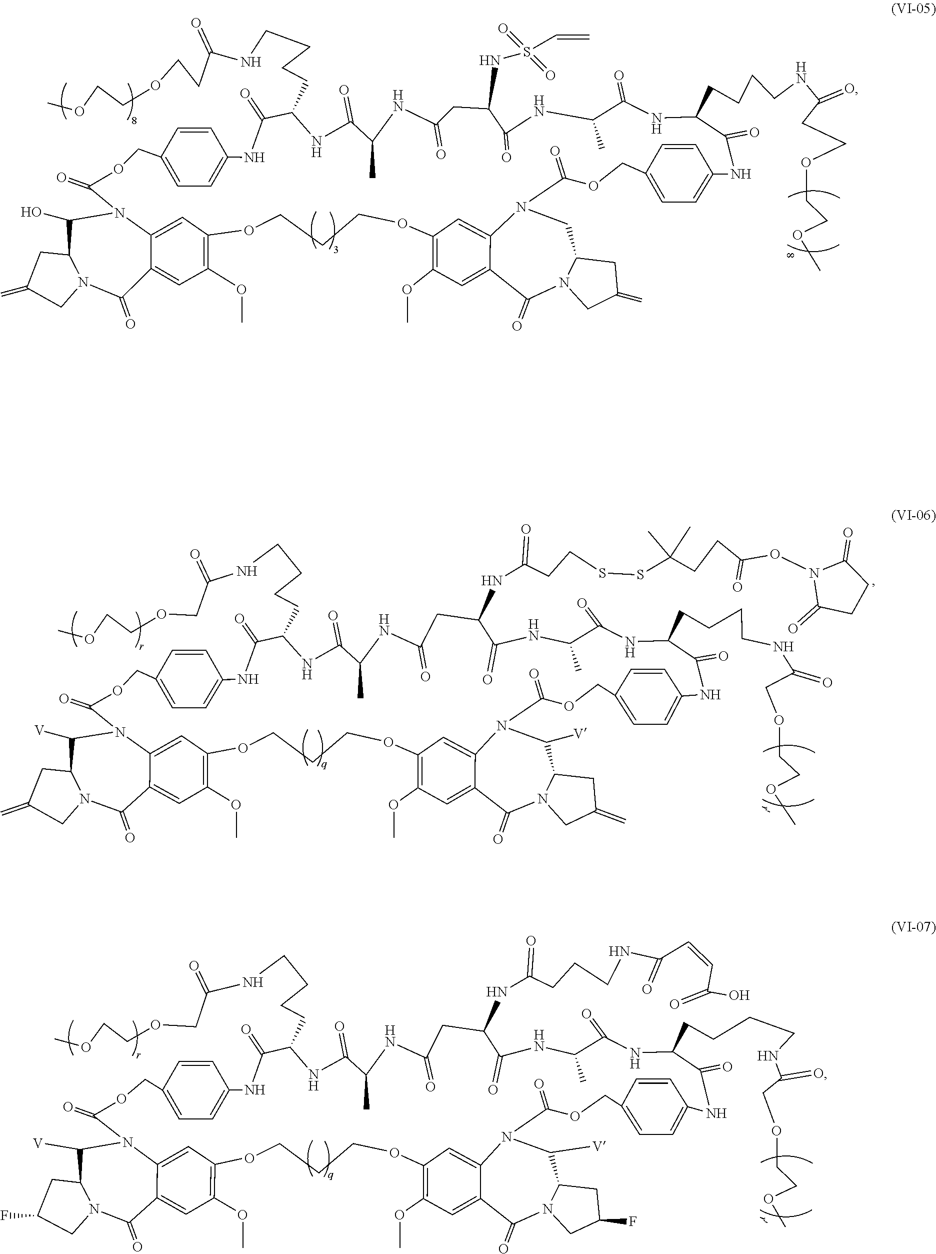 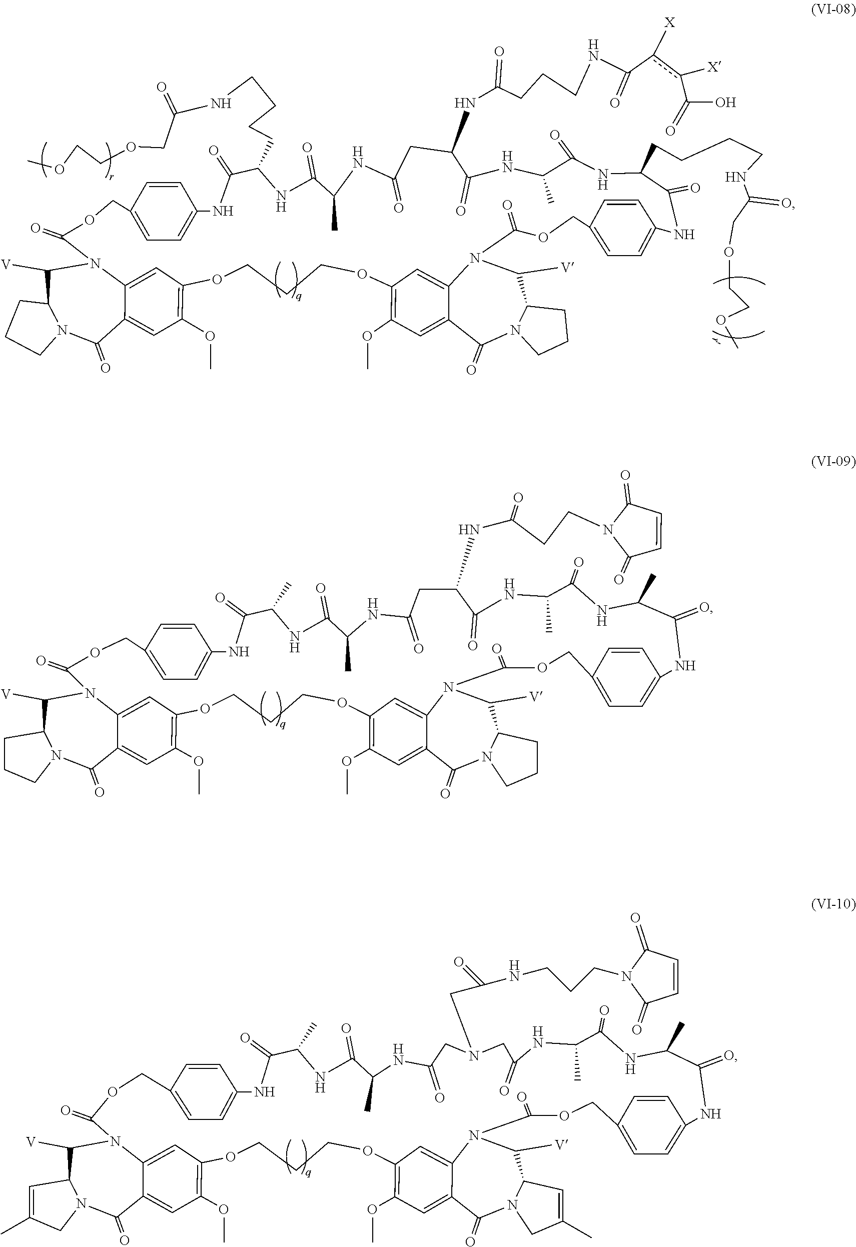 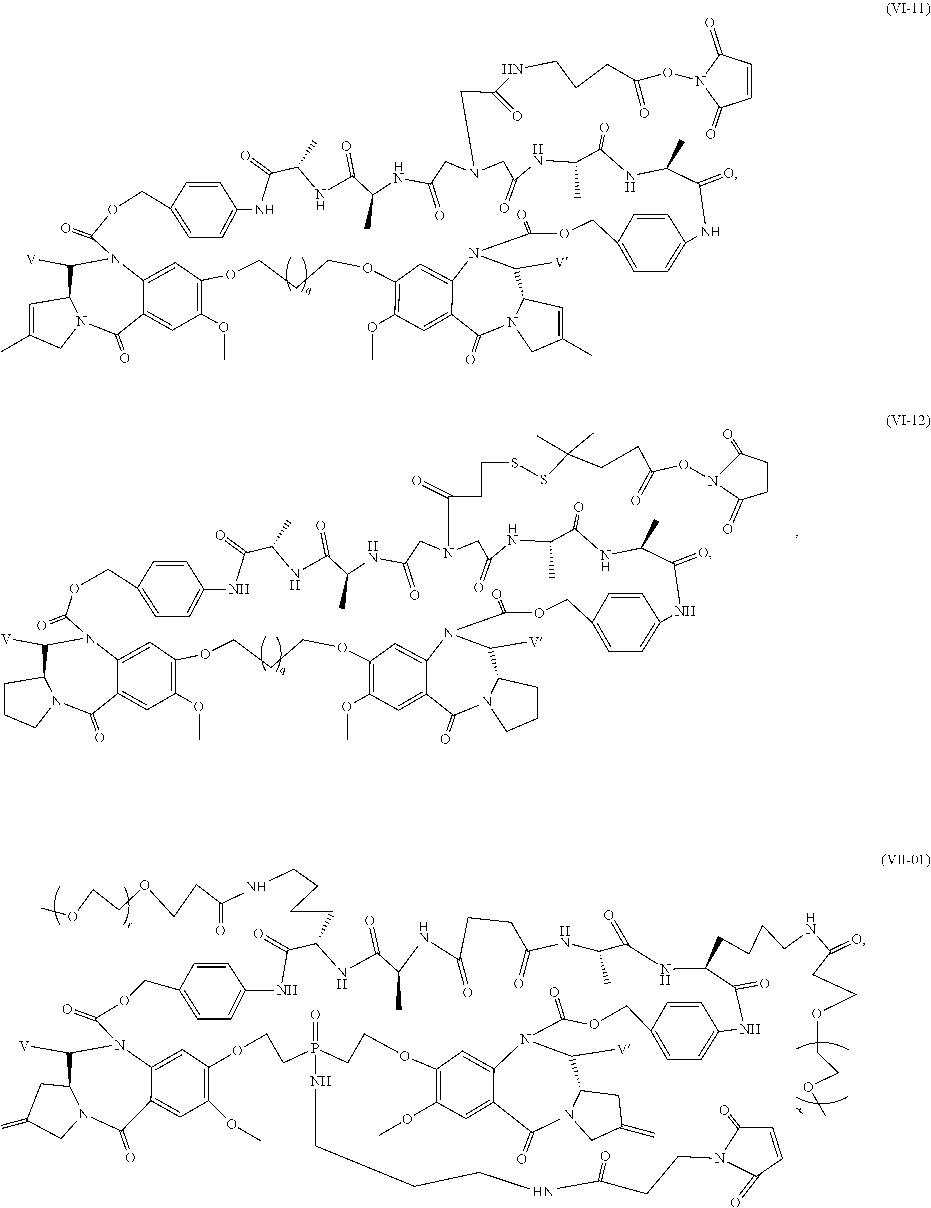 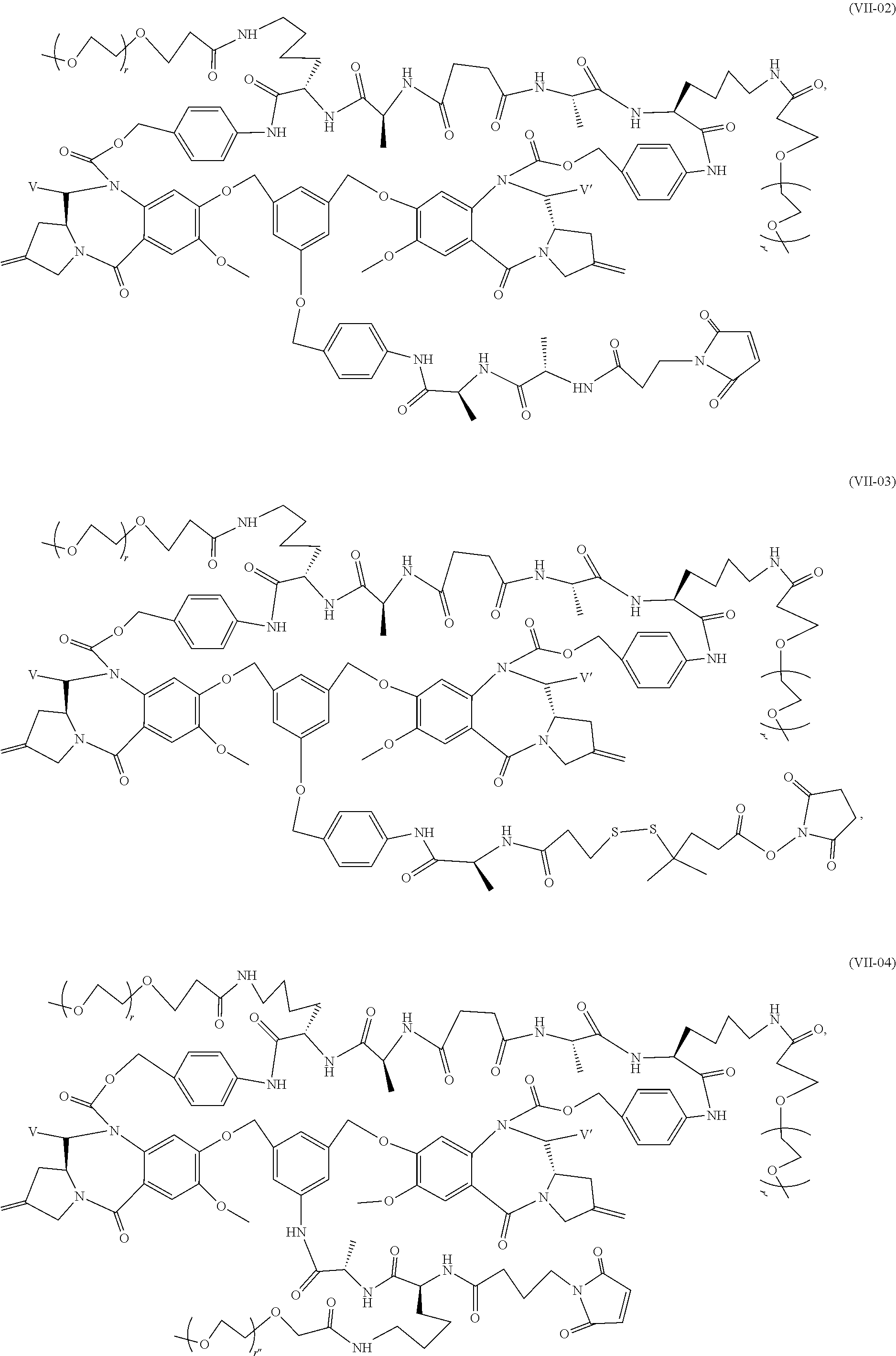 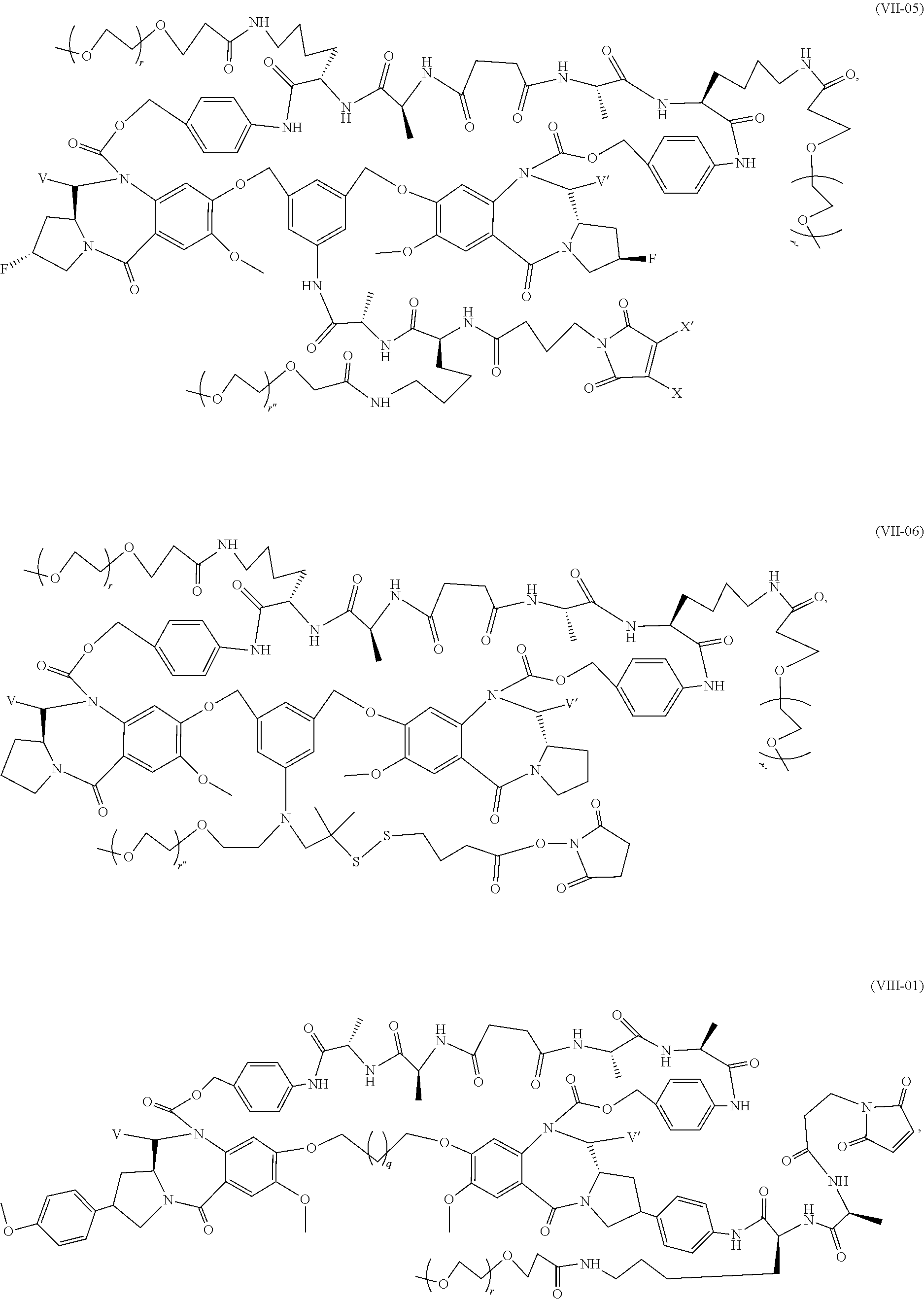 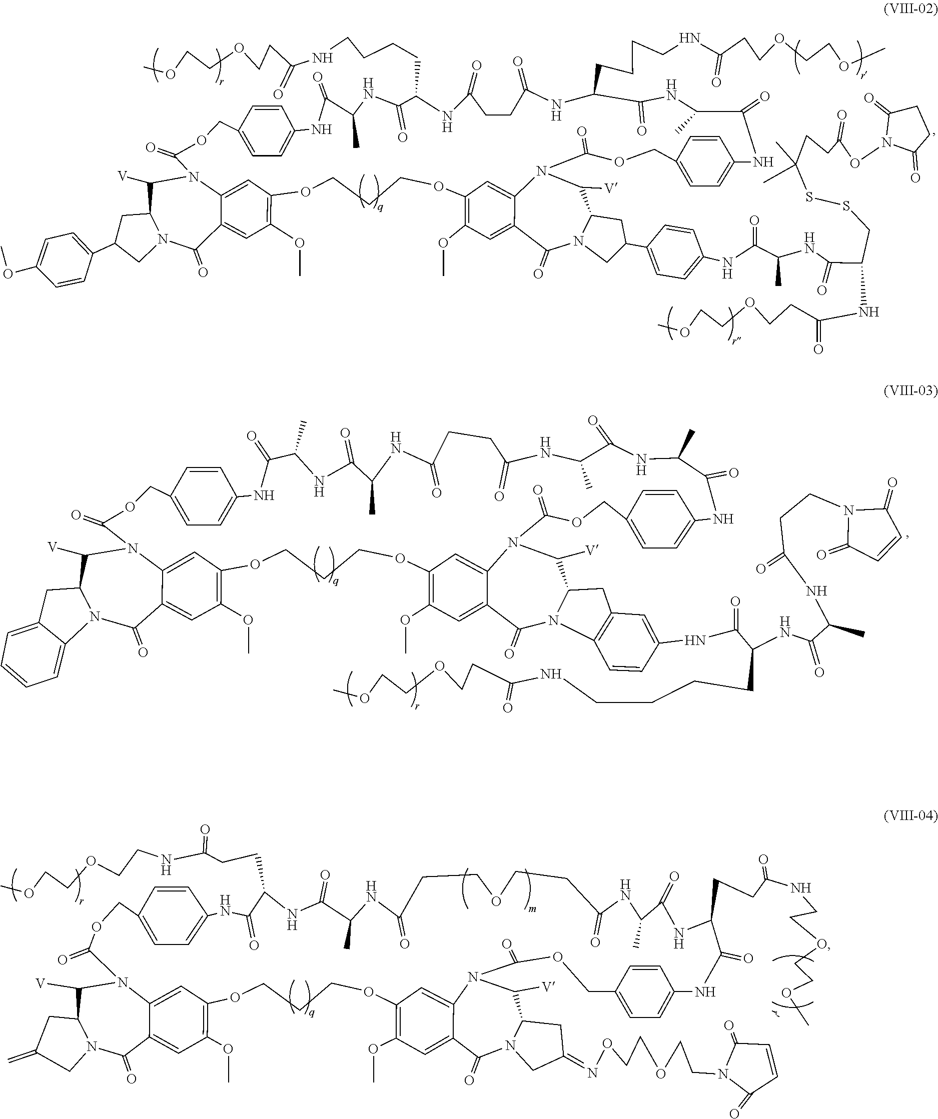 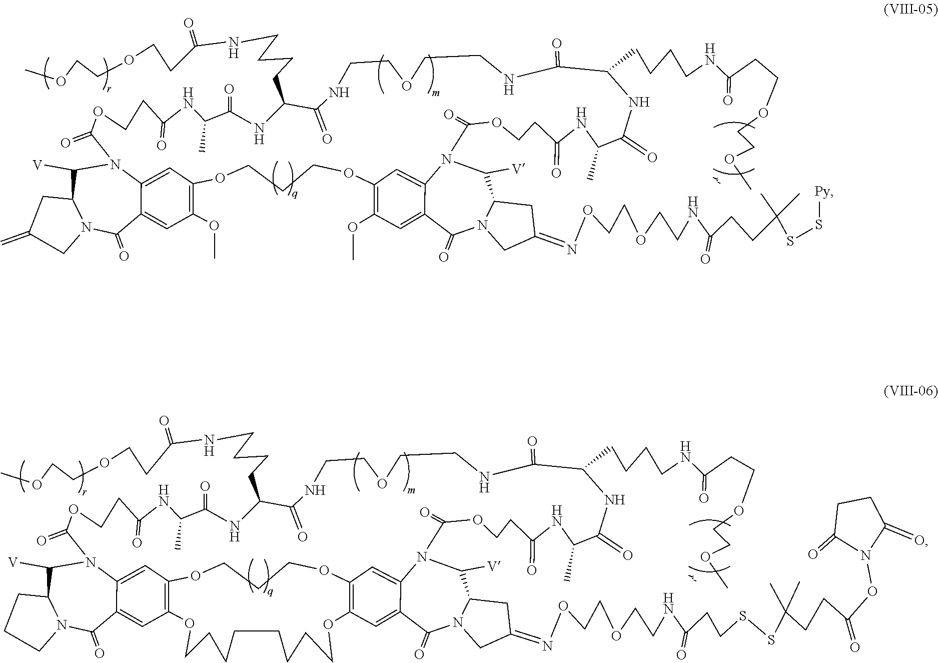
wherein U, U′, V, V′, n, n′, X, X′ and L are defined the same as in claim 6; R₆ and R₆′ are independently selected from C₁-C₆ alkyl, aryl, cyclic, cyclohetero, halogen, haloalkyl, alkoxy, haloalkoxy alkylamino, —NO₂, —CN or H; r, r₁, r₂ and r′ are independently 0-200.

8. The cross-linked PBD dimer derivative or its conjugate to a cell-binding molecule according to claim 1 having Formula 31, 45, 57, 59, 61, 67, 90, 96, 148, 162, 176, 178, 184, 188, 209, 214, 224, 232, 255, 268, 271, CC-3, CC-4, CC-5, CC-6, CC-7, CC-8, CC-10, CC-11, CC-12, CC-14, CC-15, CC-17, CC-18, CC-20, CC-21, CC-27, or CC-29, shown below: 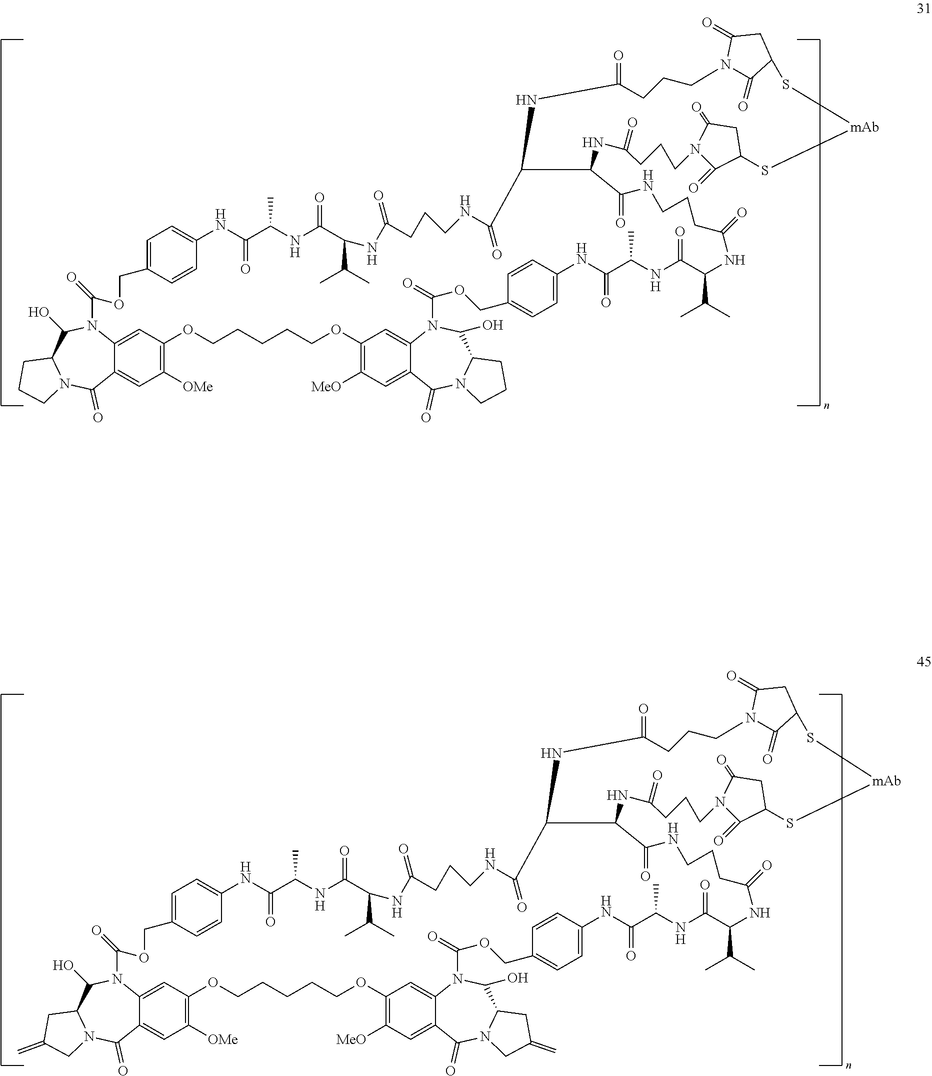 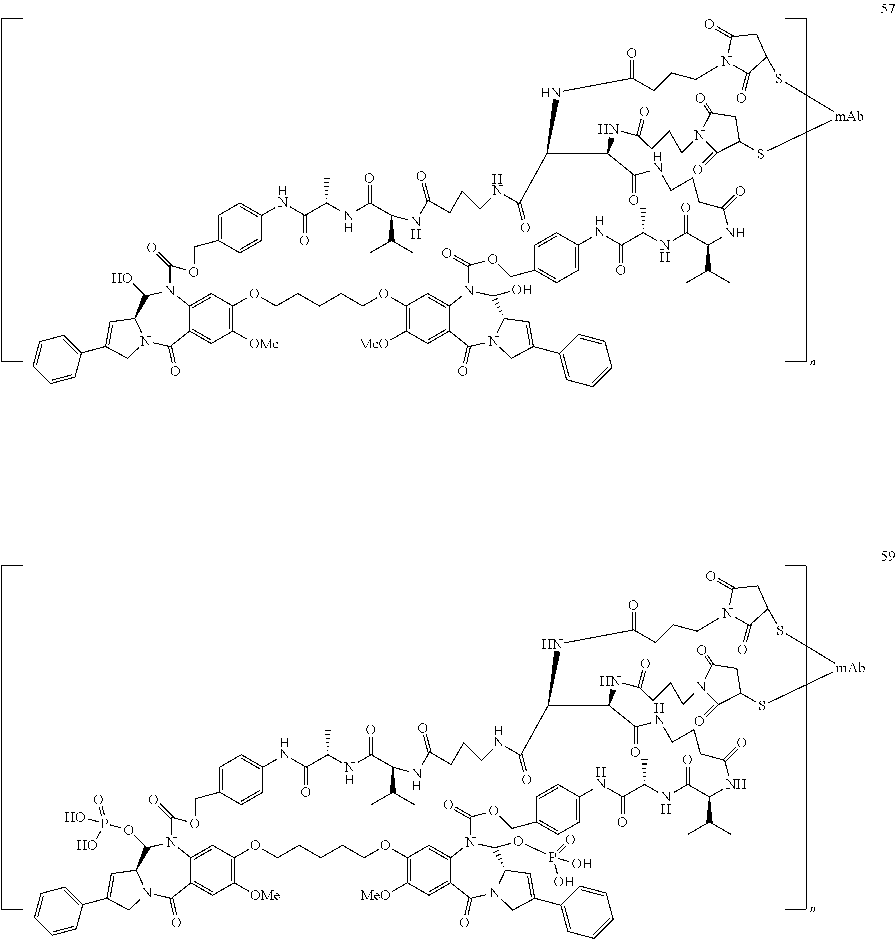 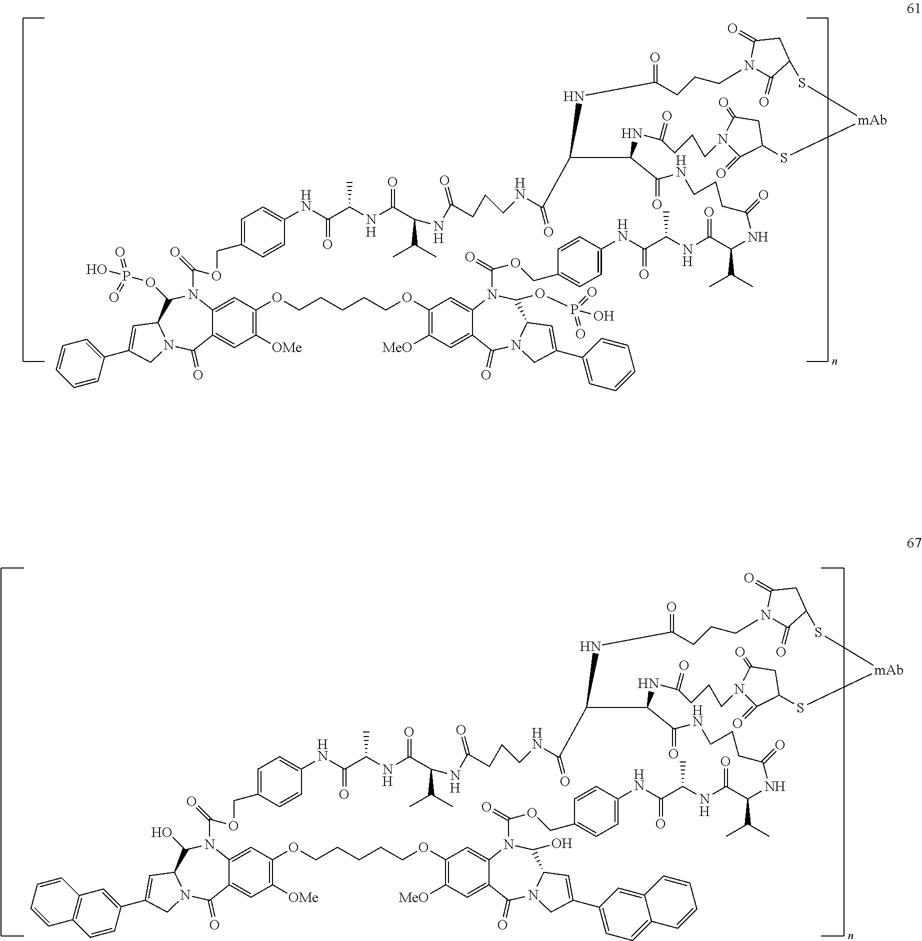 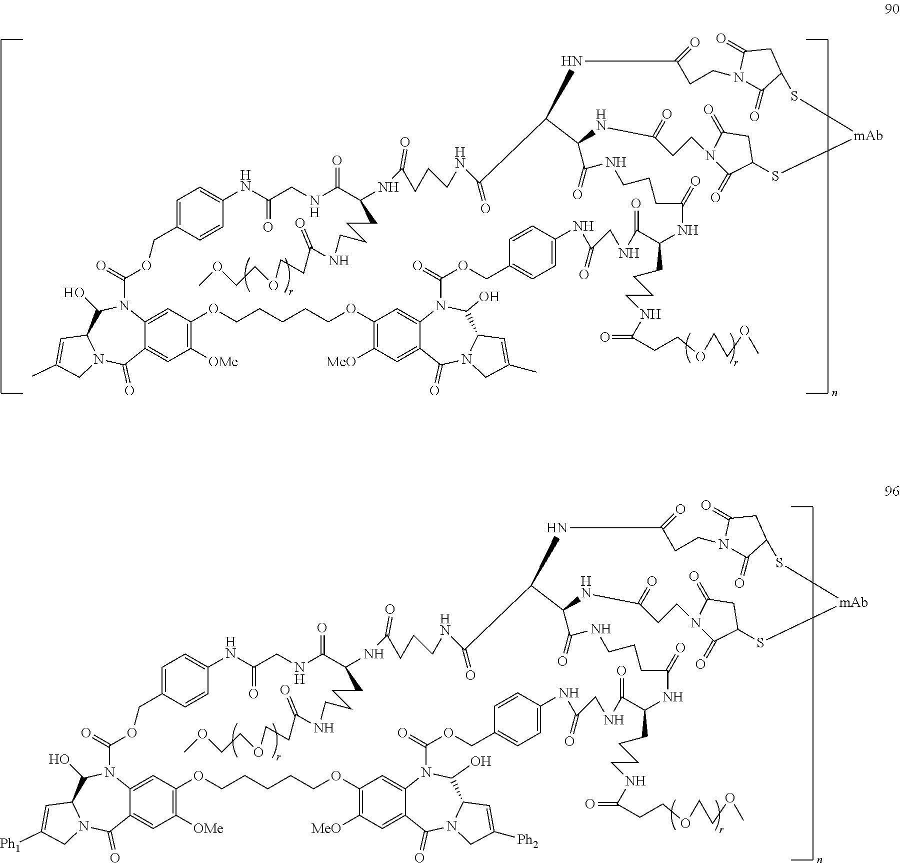 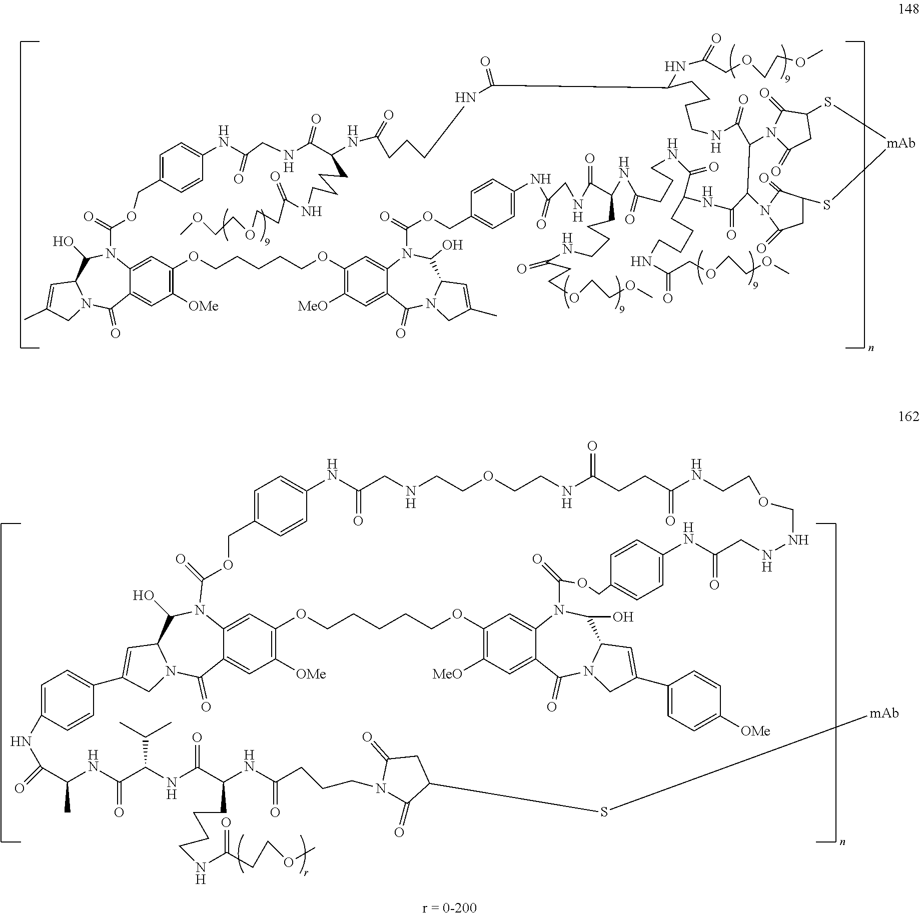 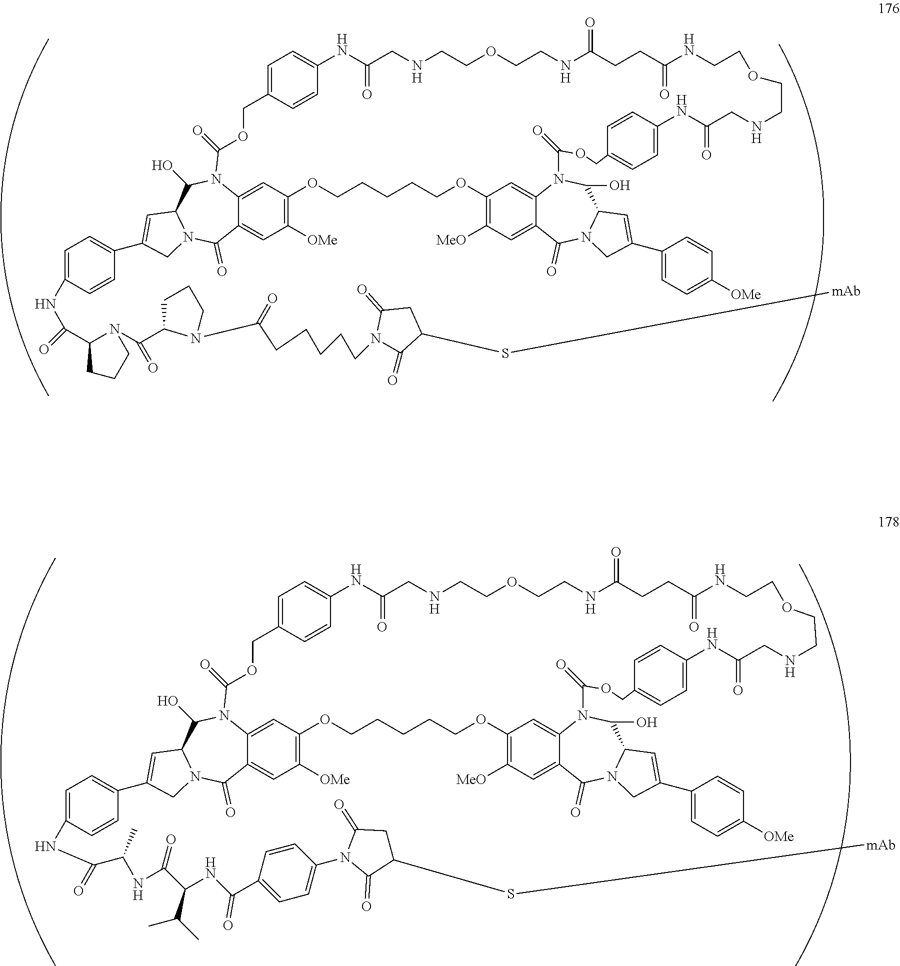 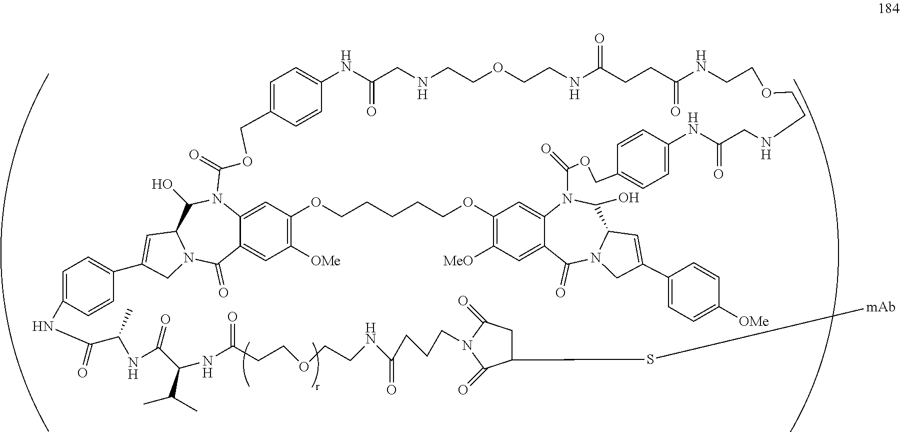 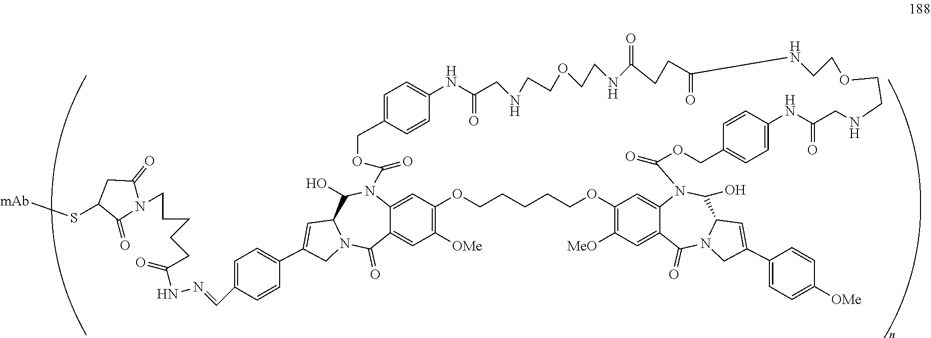 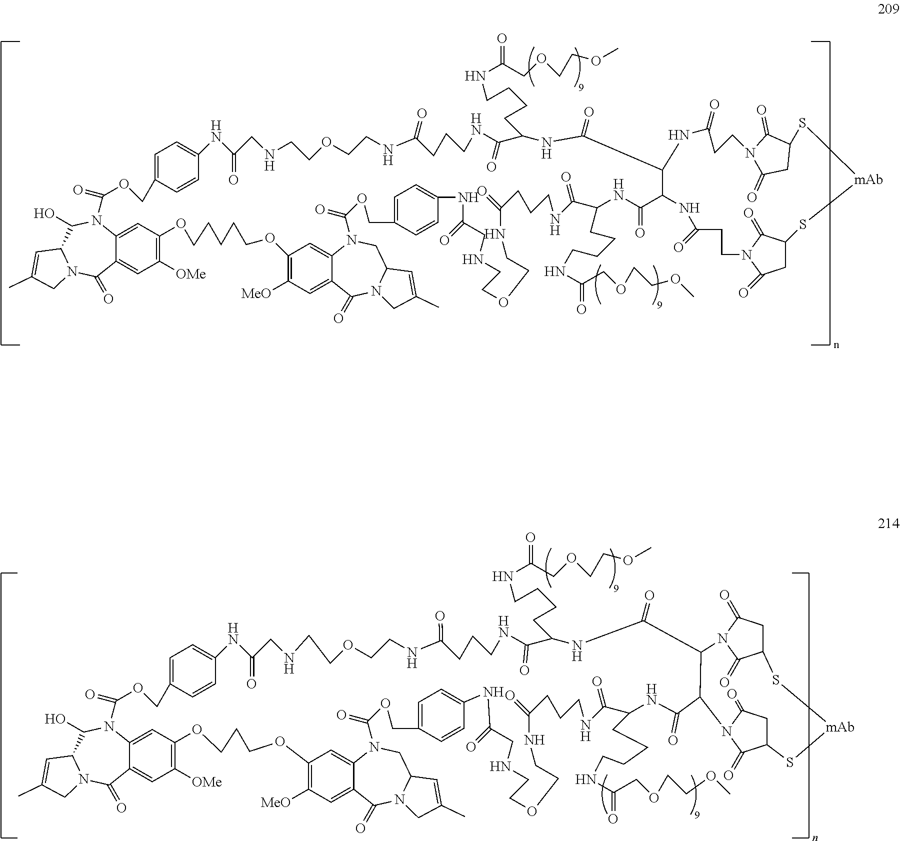 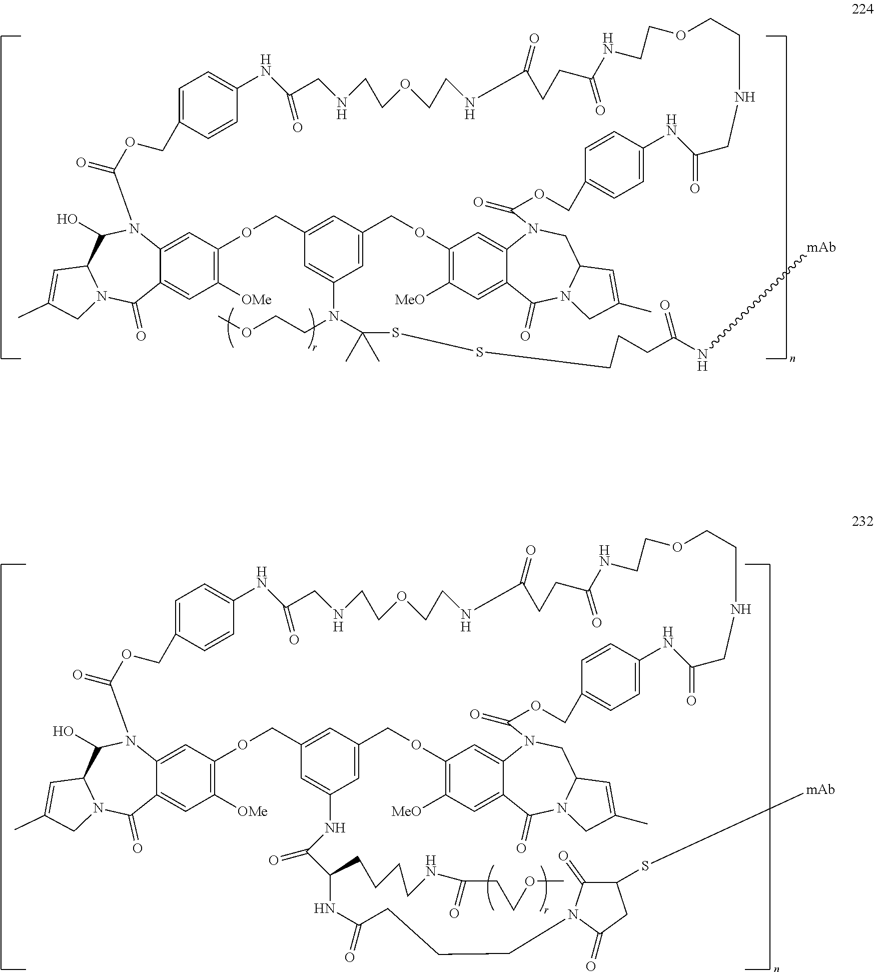 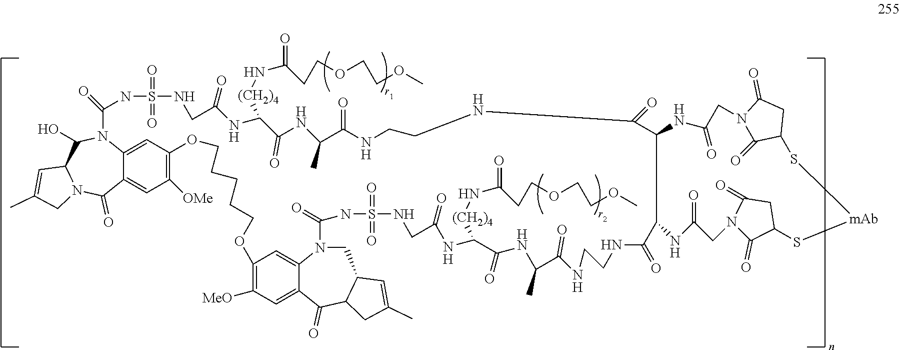 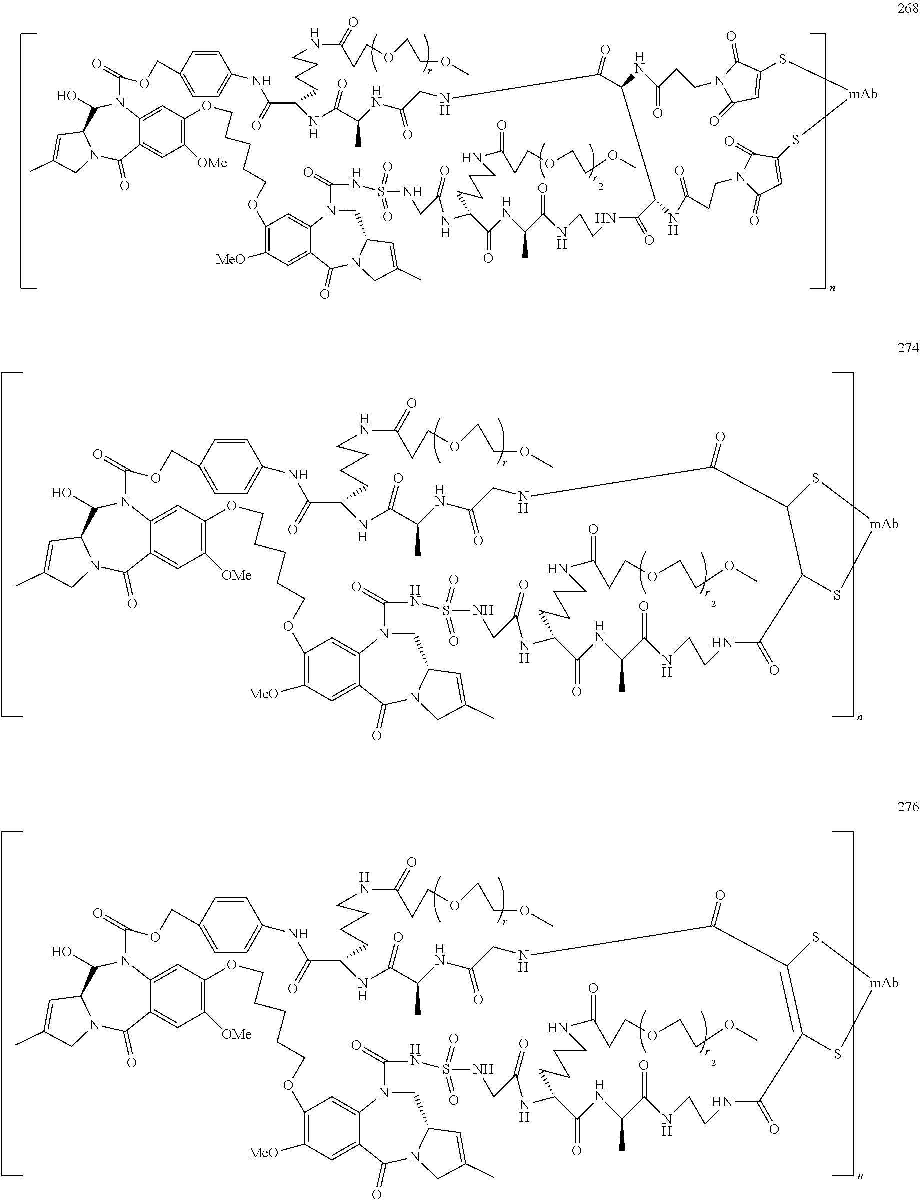 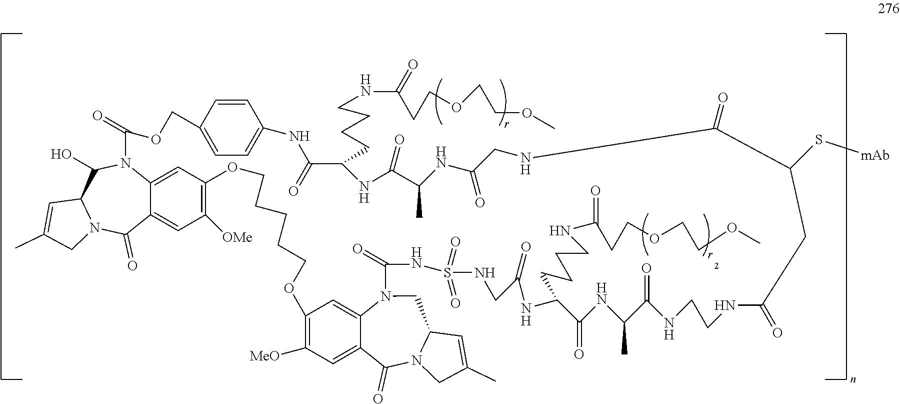 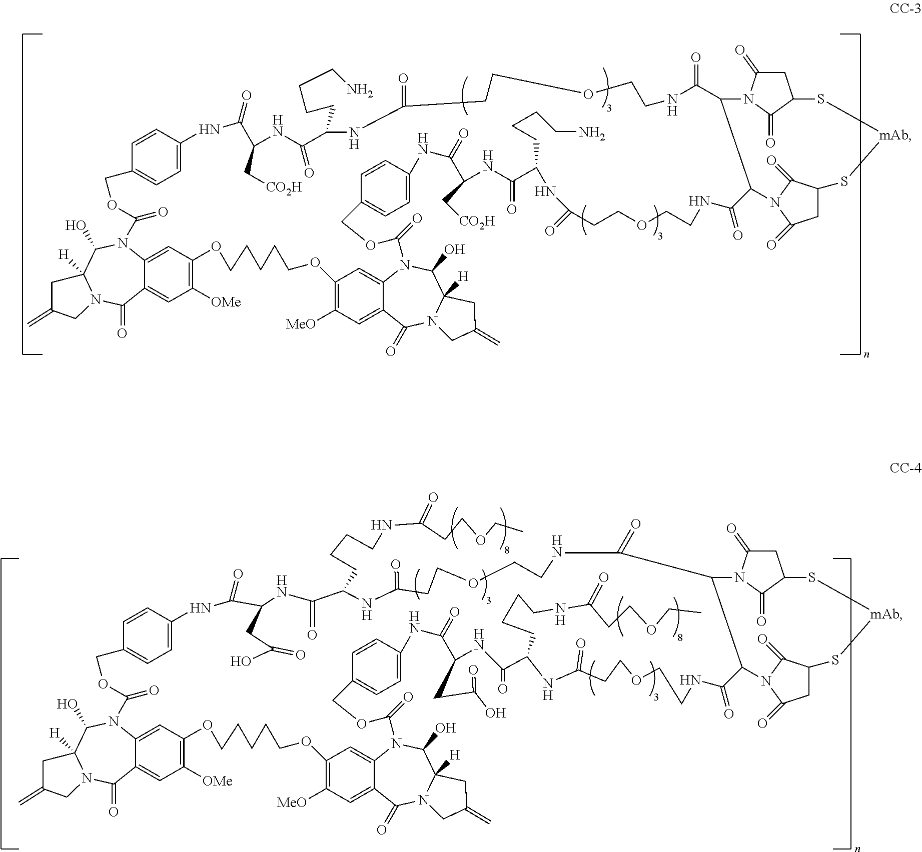 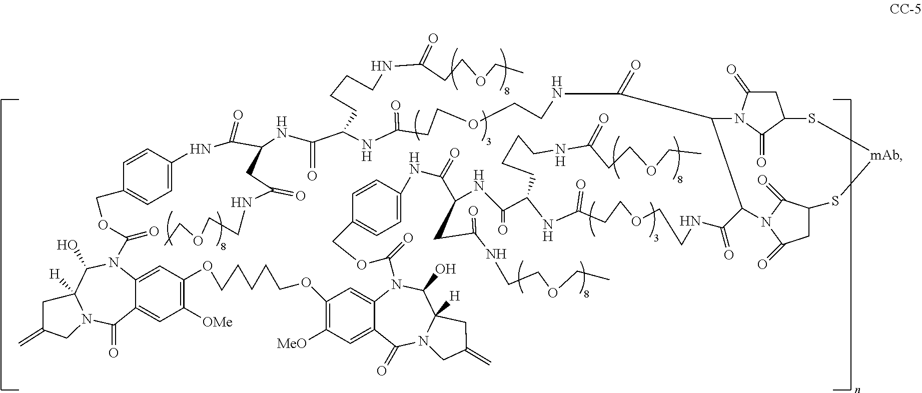 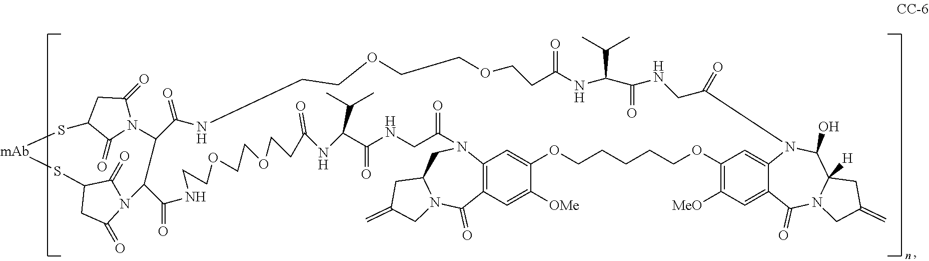 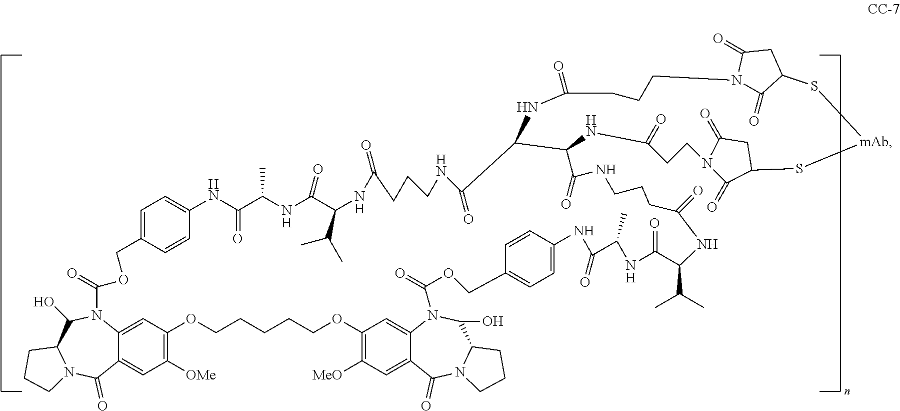 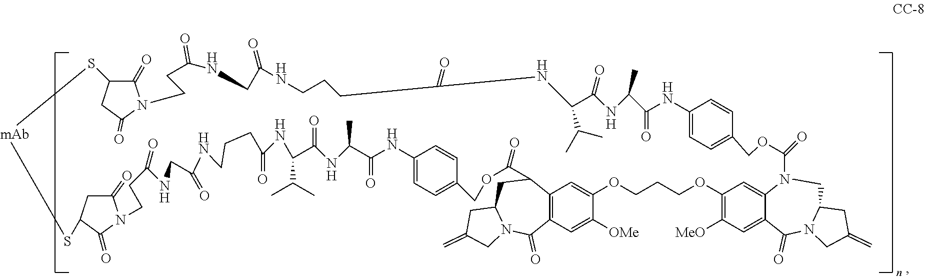 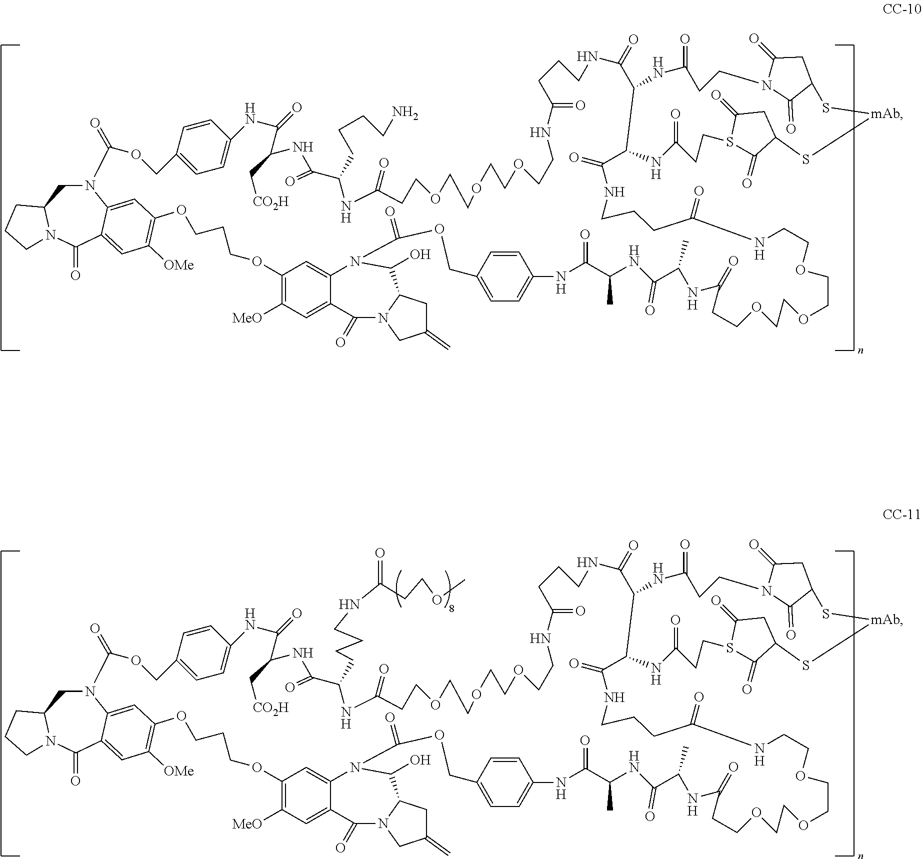 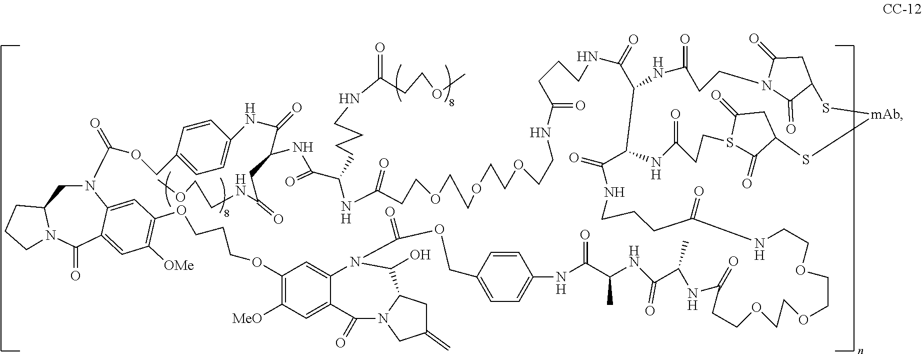 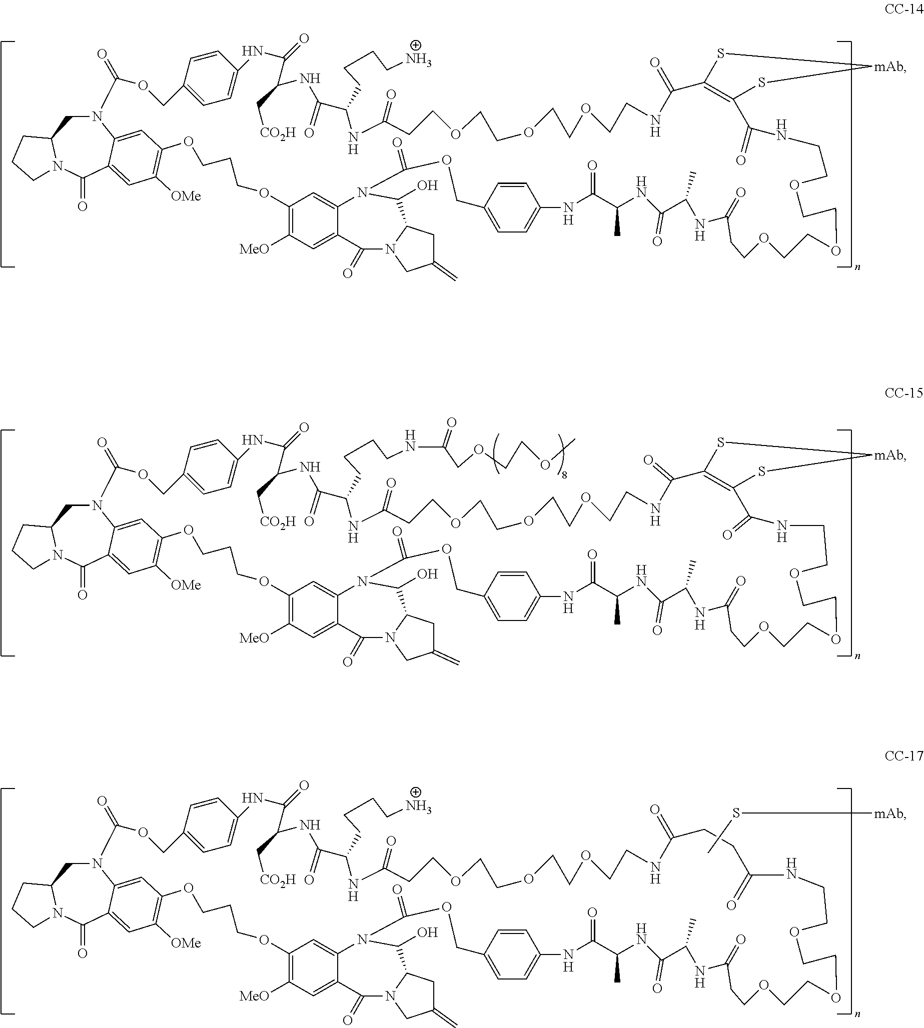 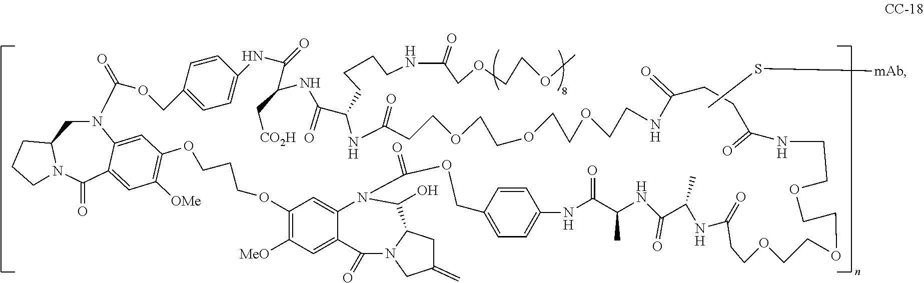 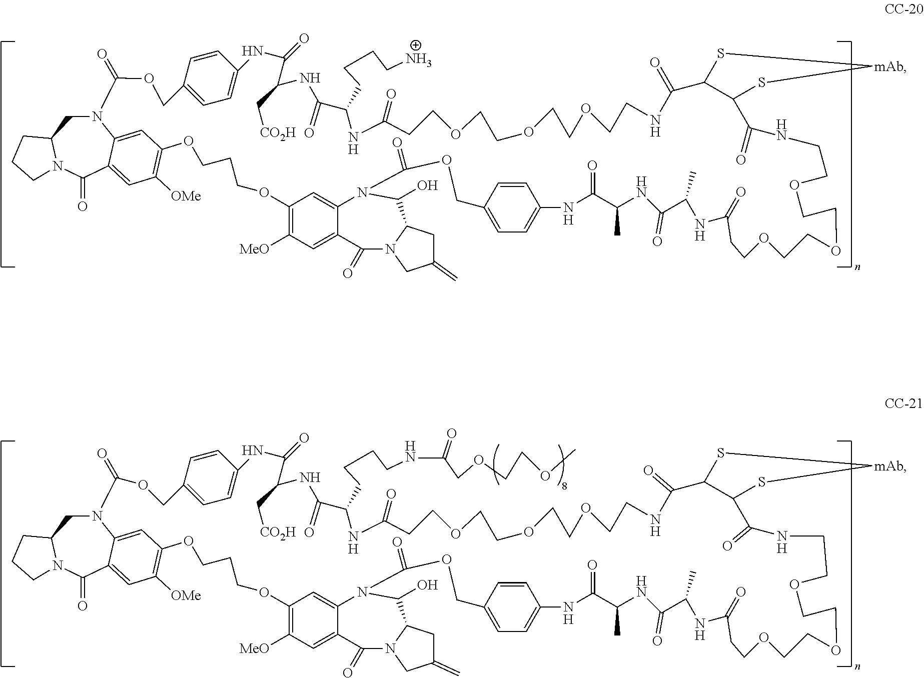 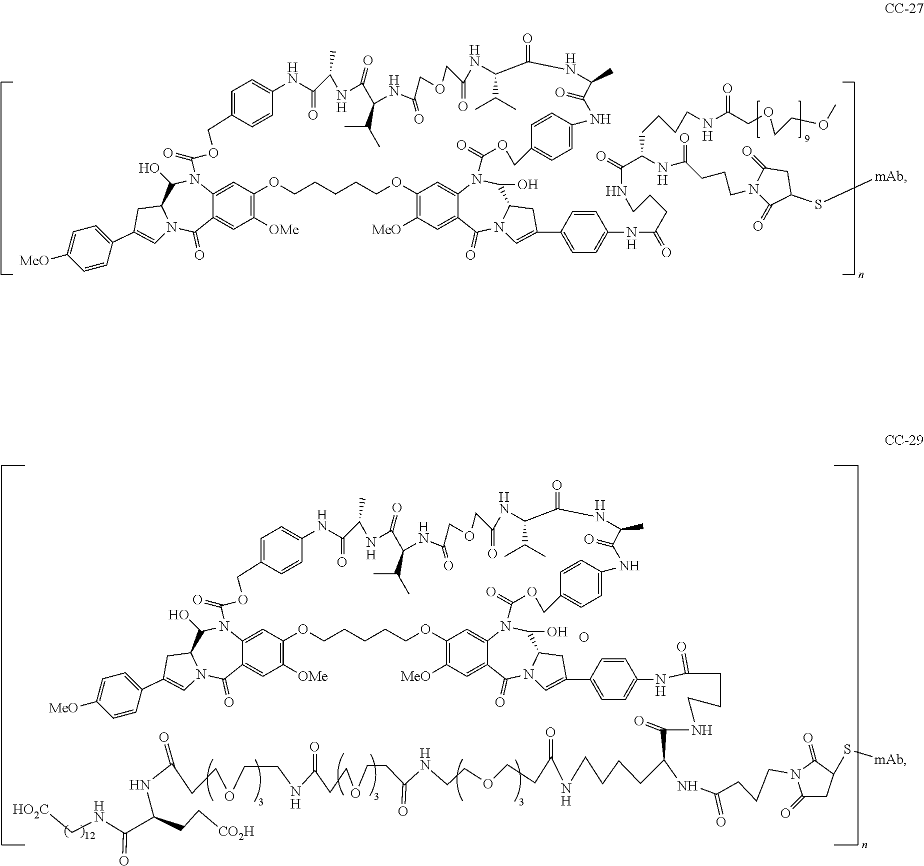
wherein r, r₁, r₂ and r′ are independently 0-200; n is 1-30.

9. The cross-linked PBD dimer derivative or its conjugate to a cell-binding molecule according to claim 6 having Formula 30, 44, 56, 58, 60, 66, 89, 95, 147, 161, 175, 177, 183, 187, 208, 213, 223, 231, 254, 267, 270, 273, 275, 277, C-3, C-4, C-5, C-6, C-7, C-8, C-10, C-11, C-12, C-14, C-15, C-17, C-18, C-20, C-21, C-27, or C-29 below: 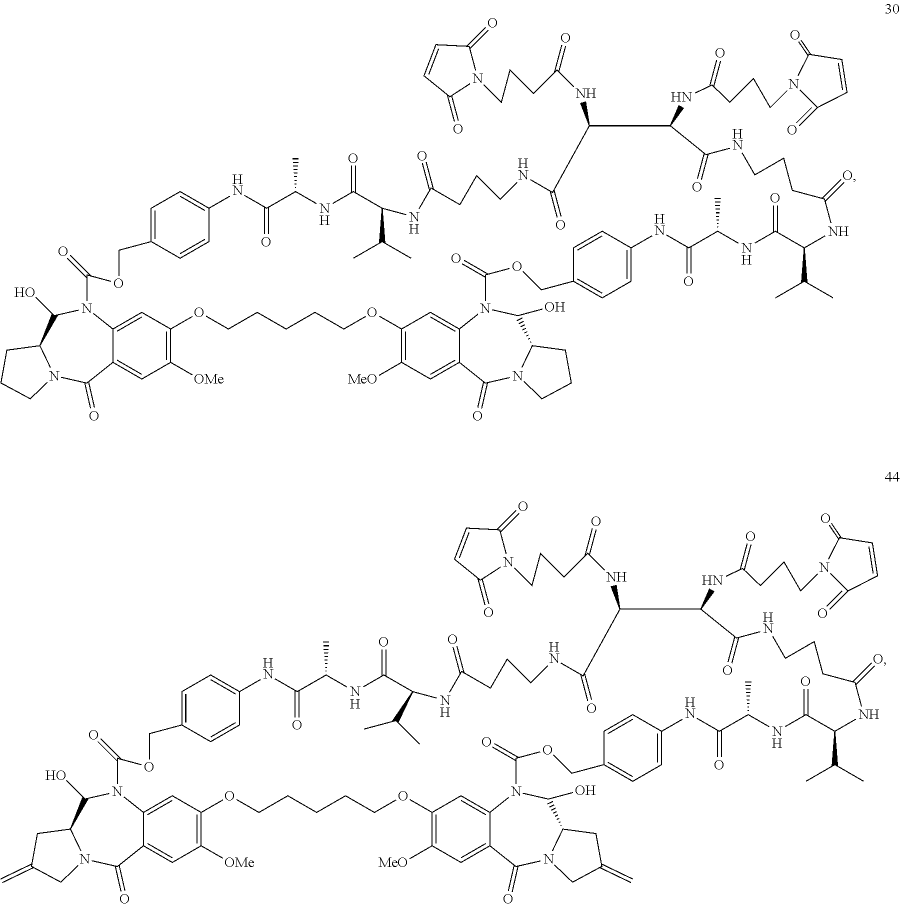 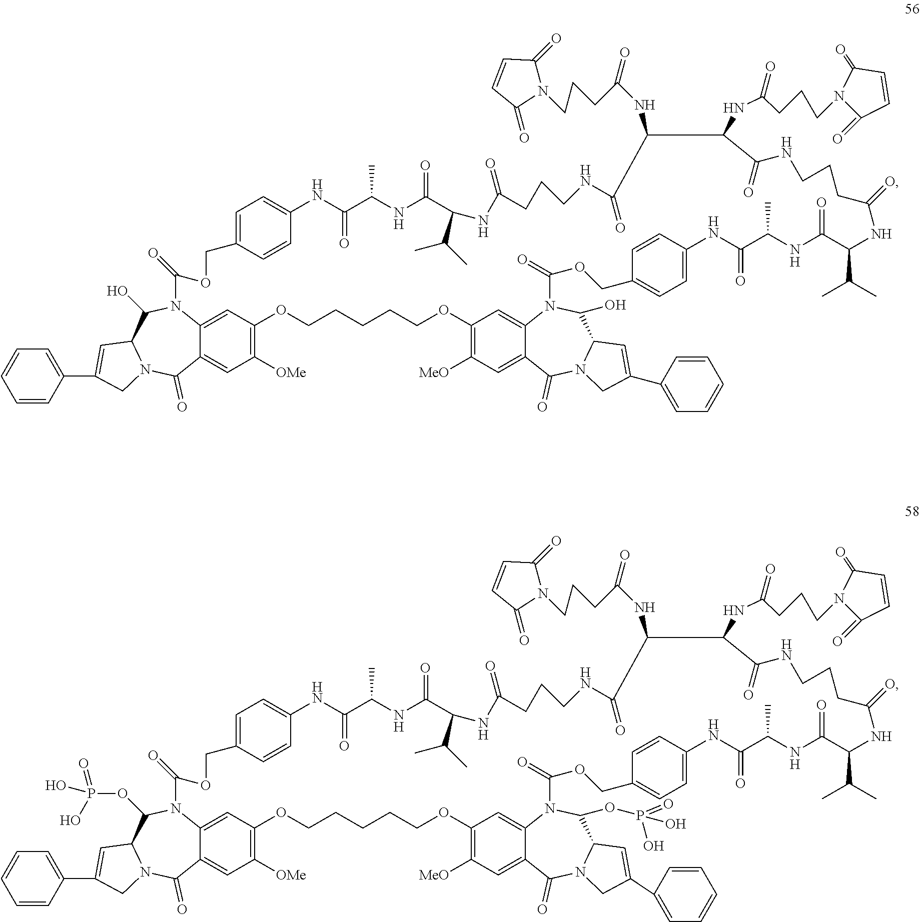 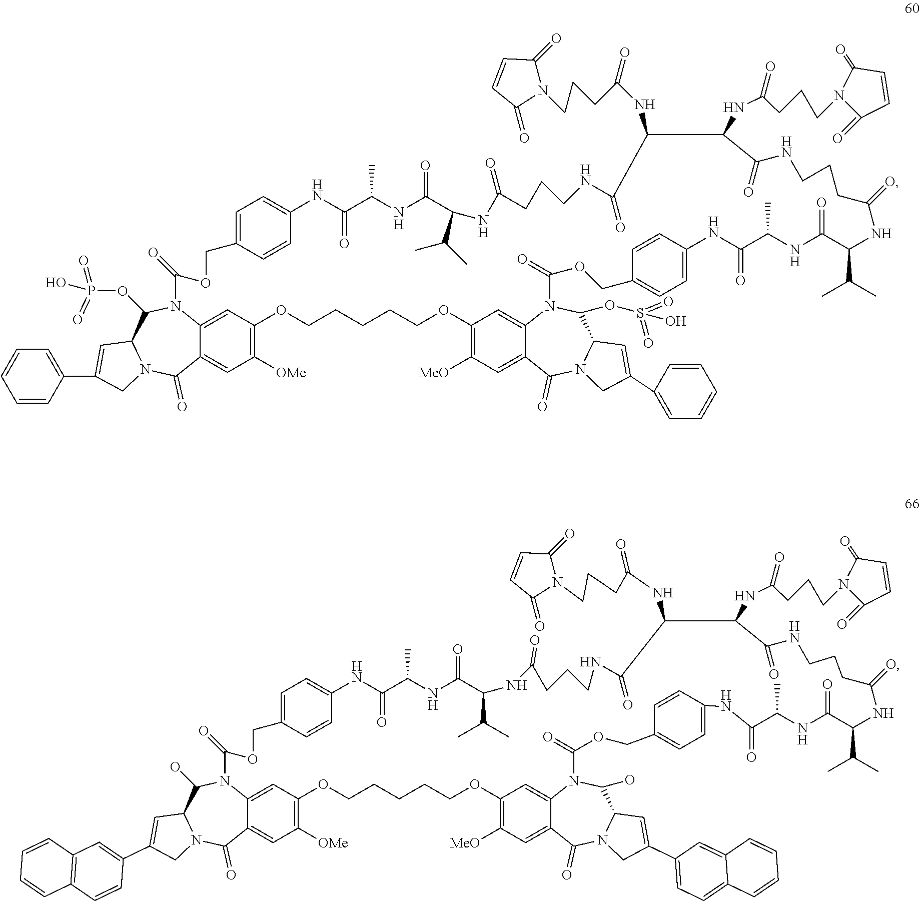 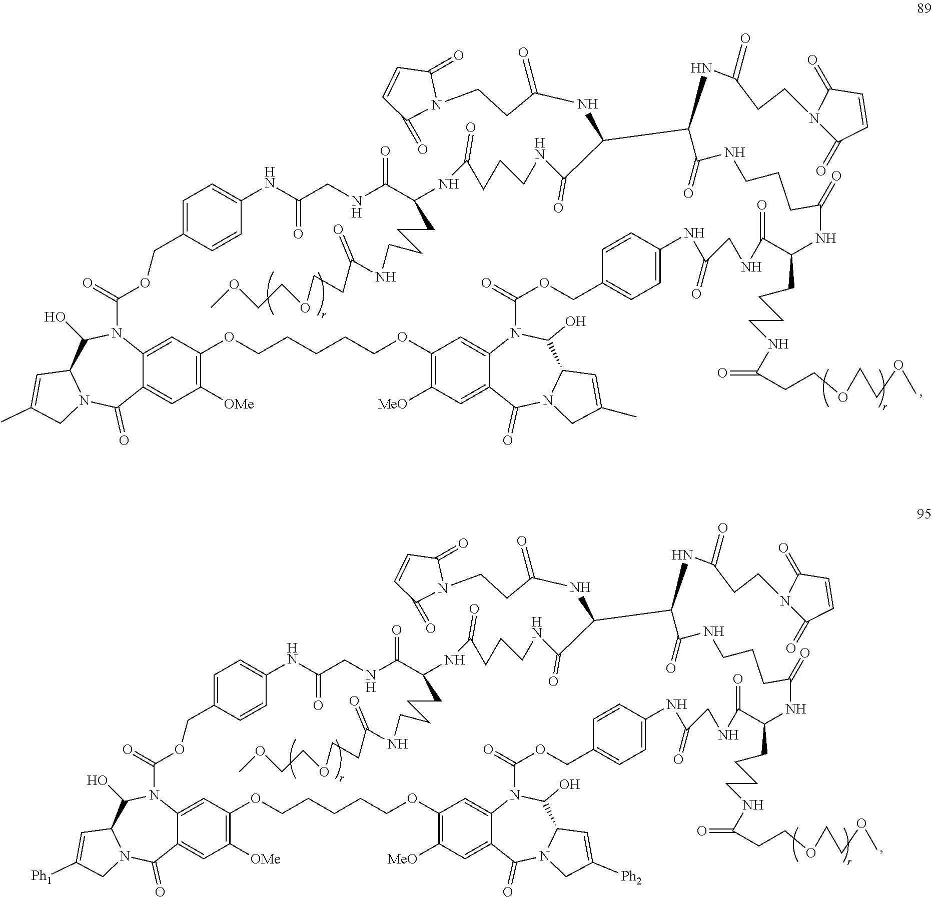 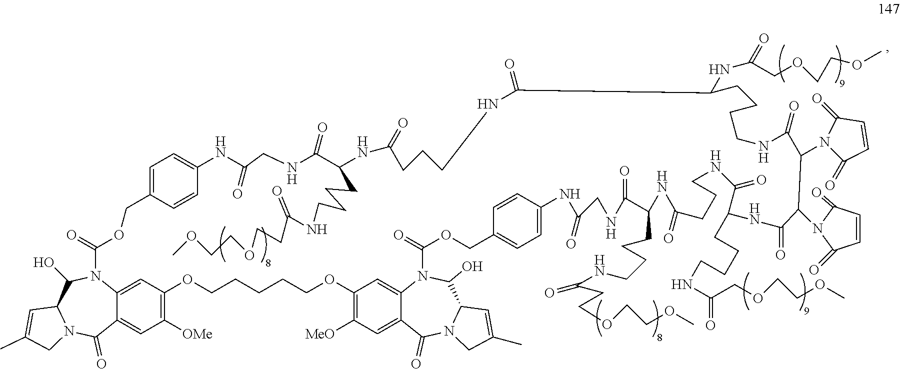 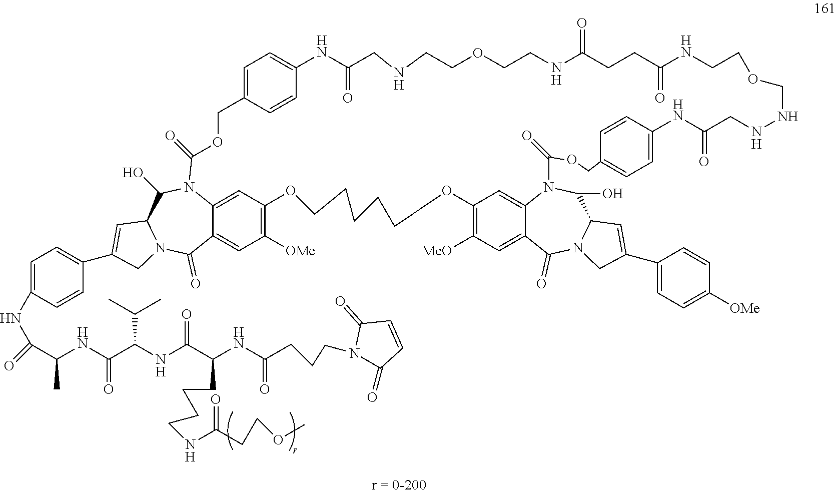 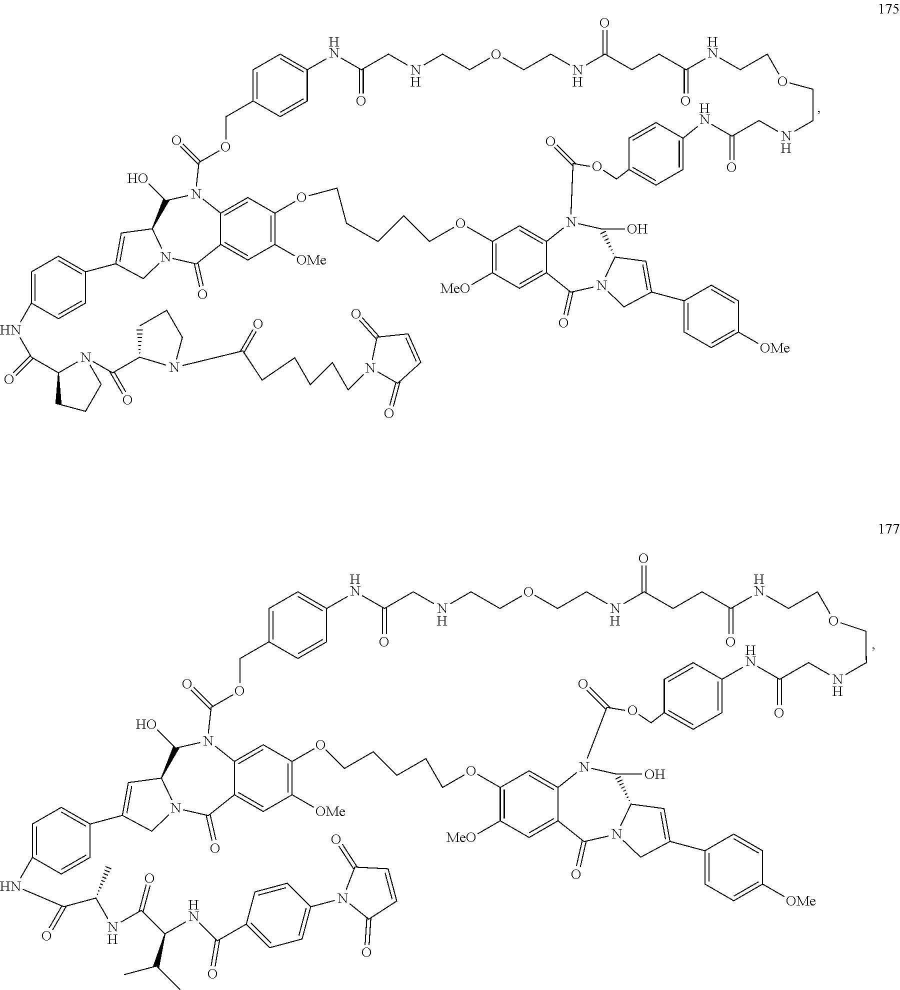 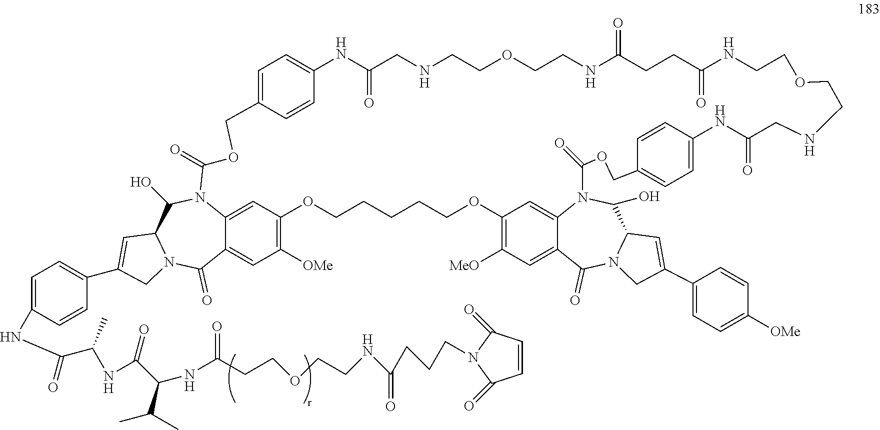 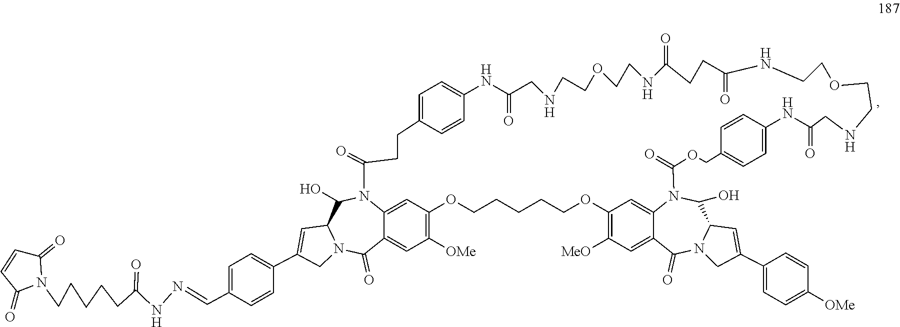 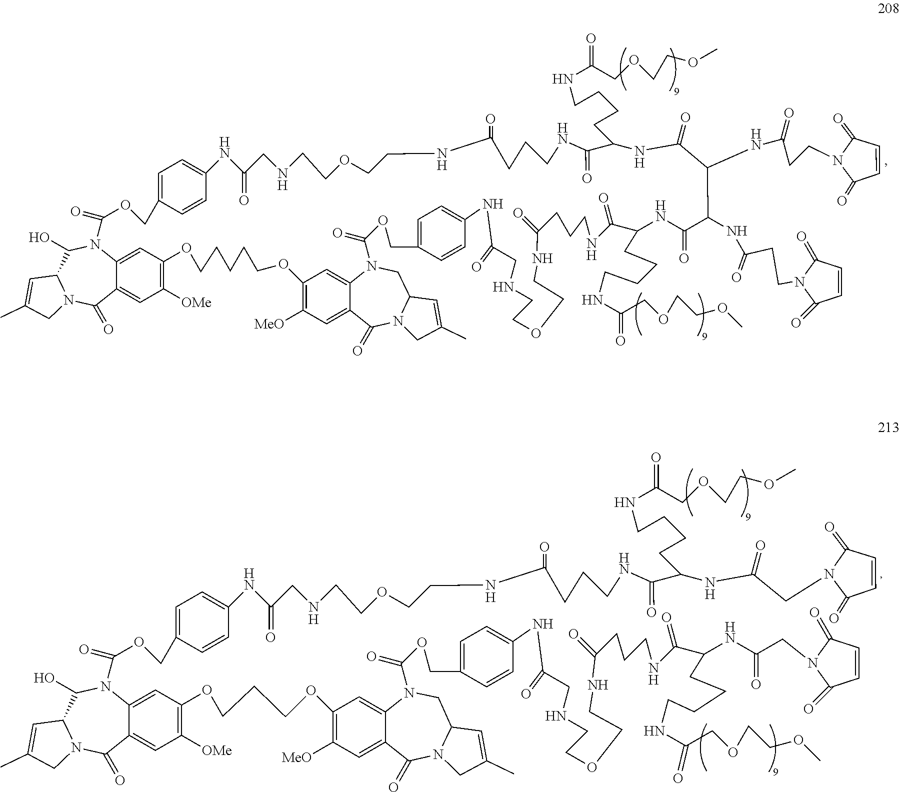 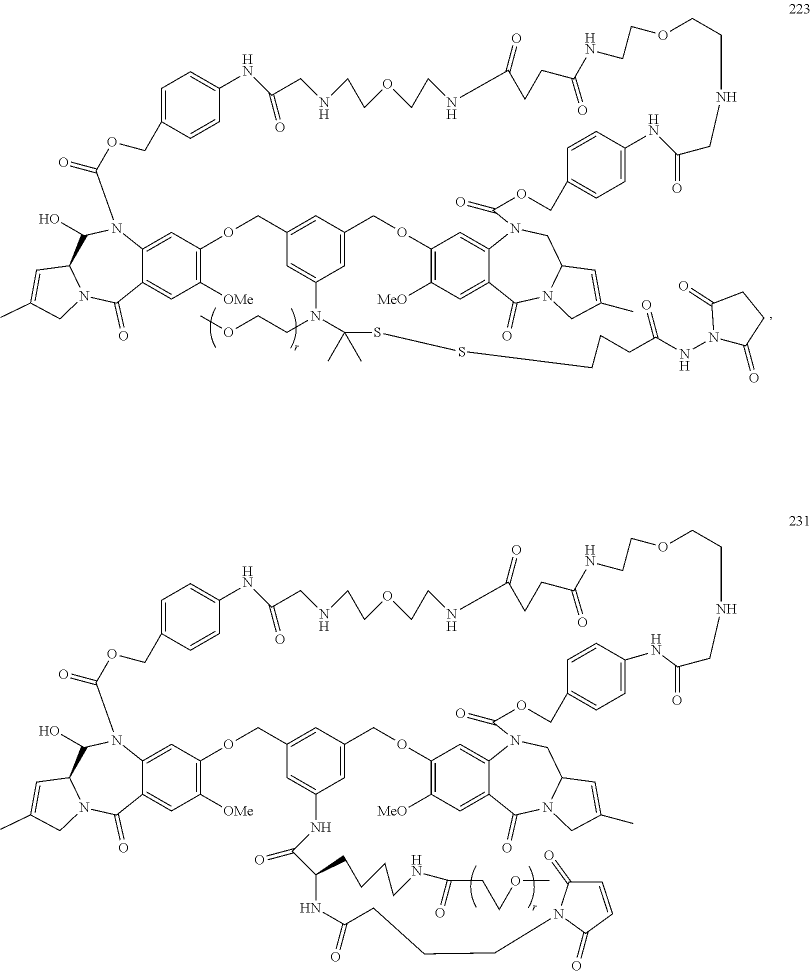 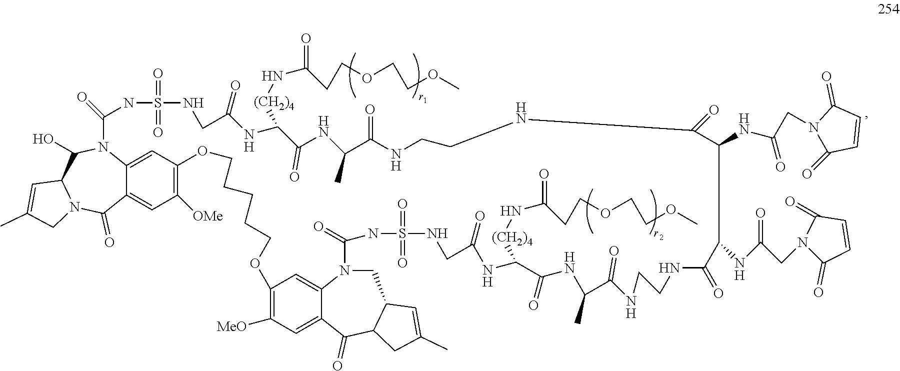 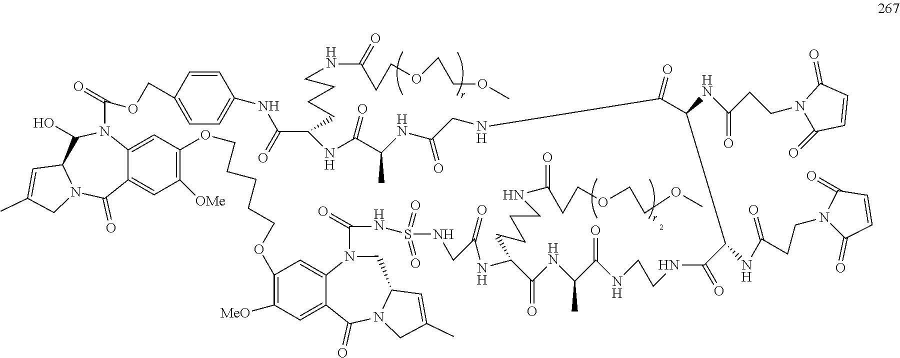 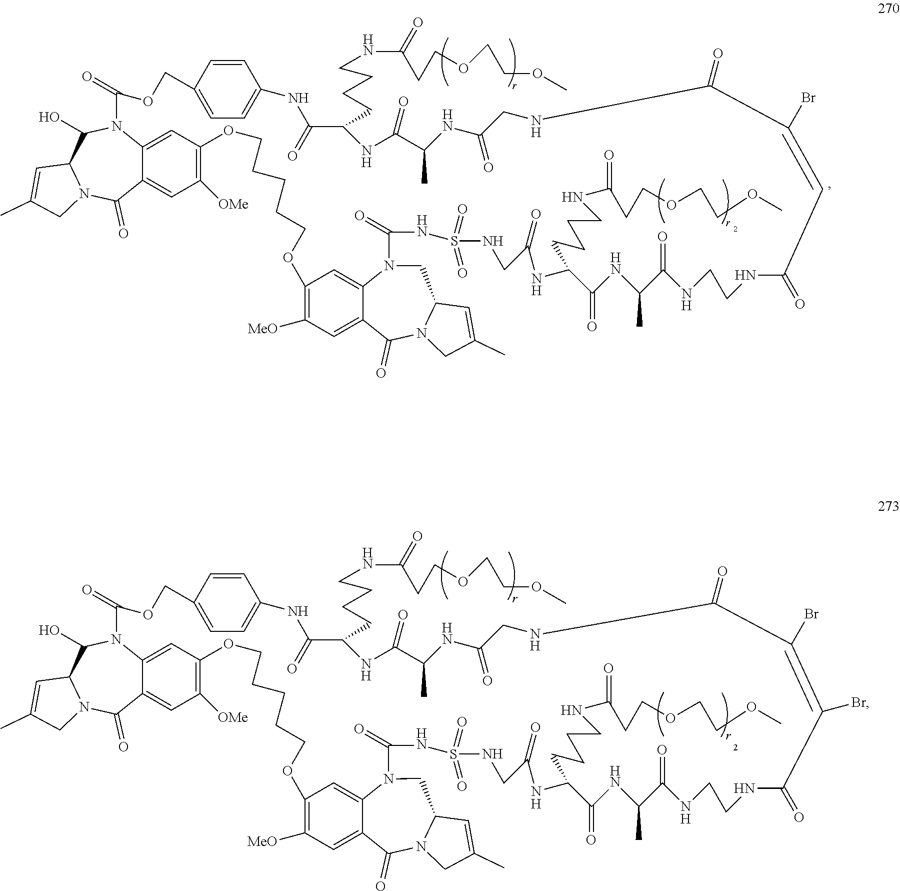 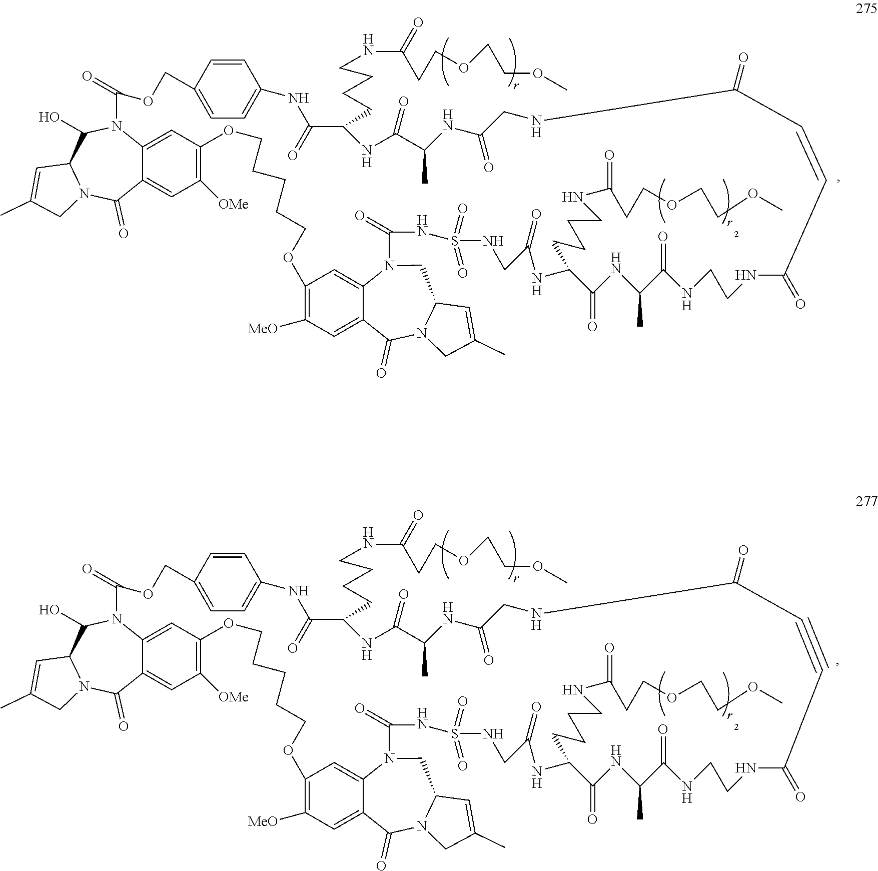 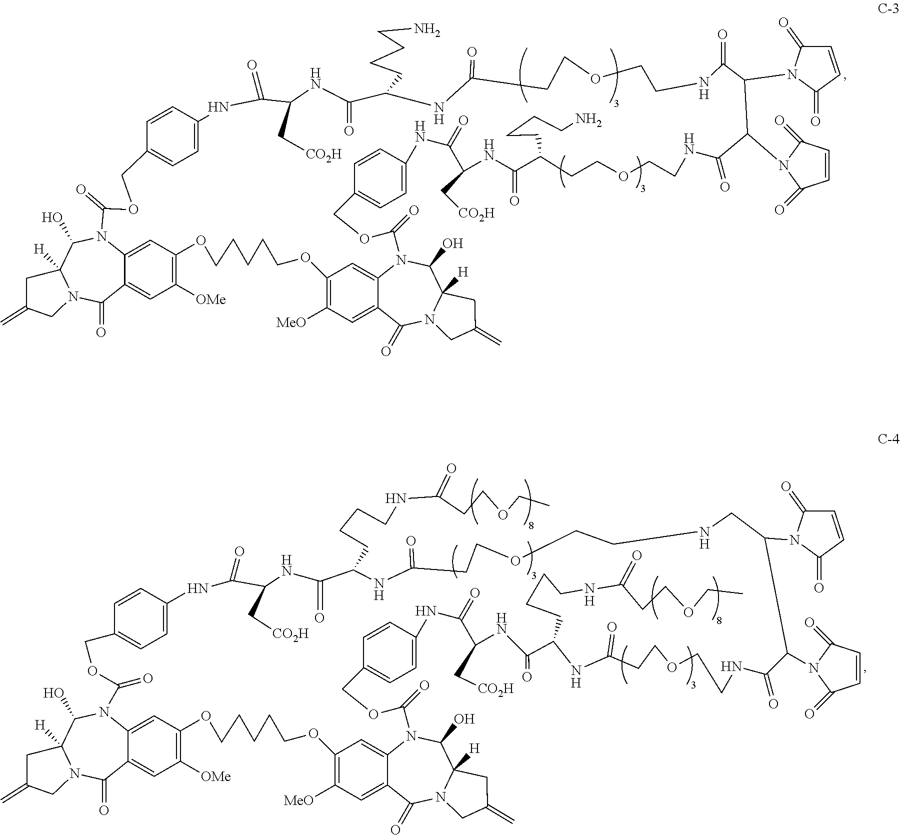 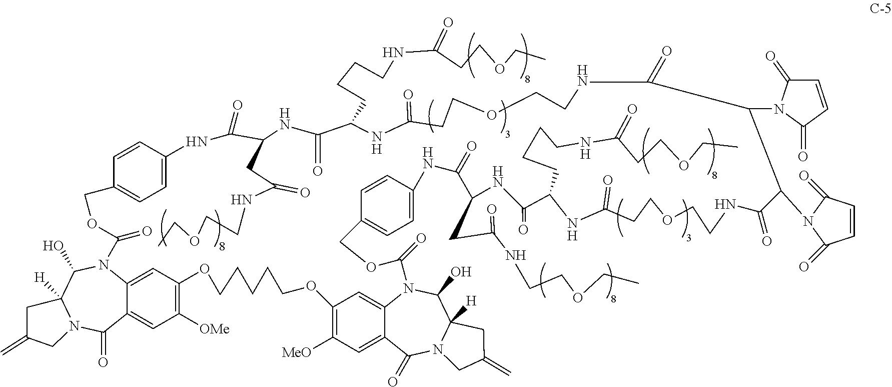 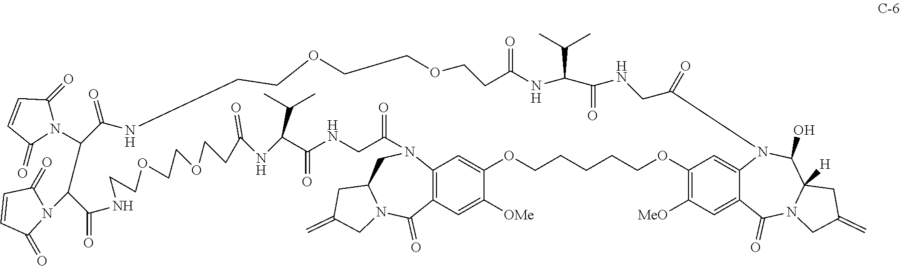 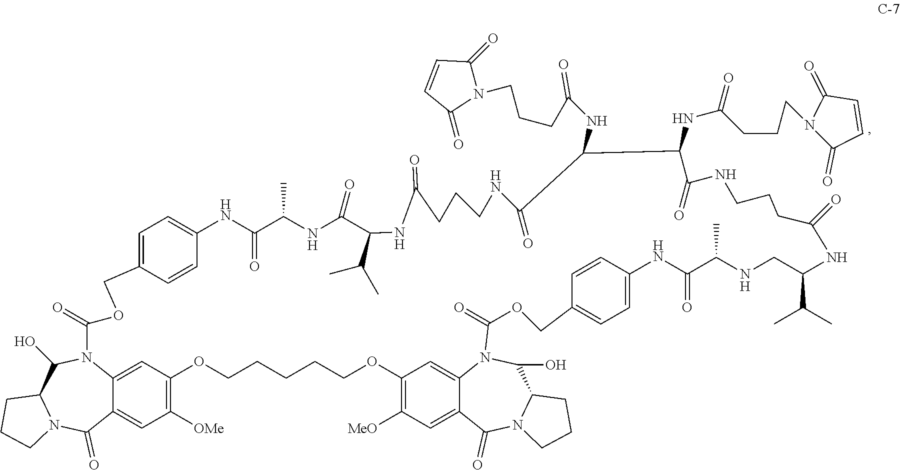 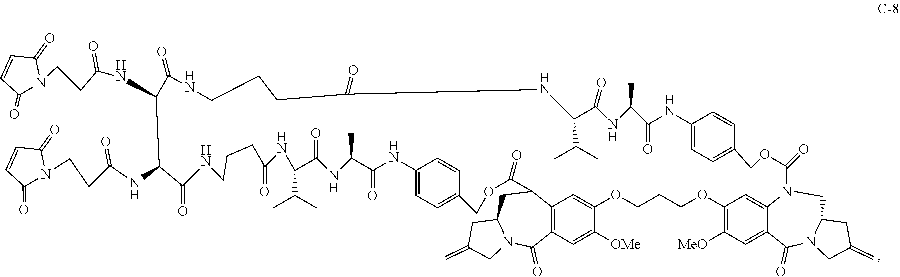 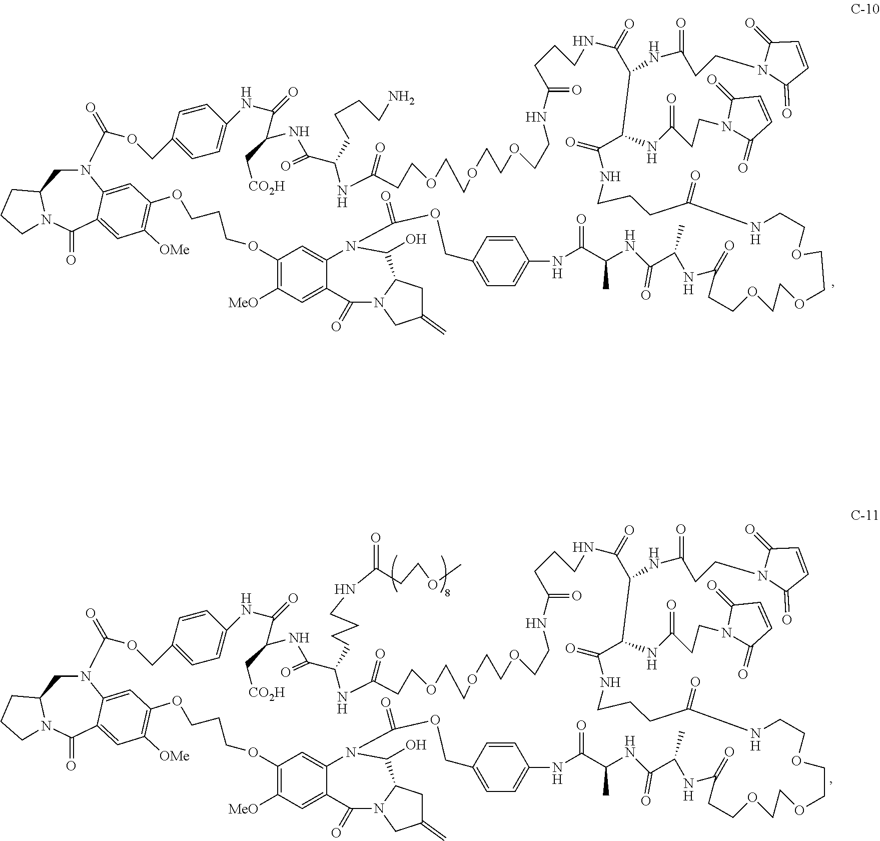 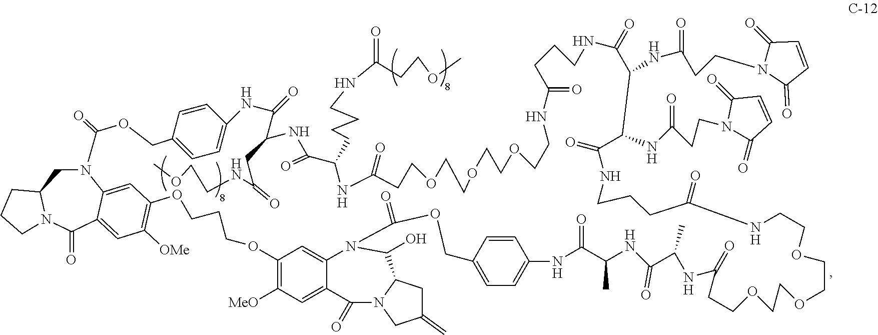 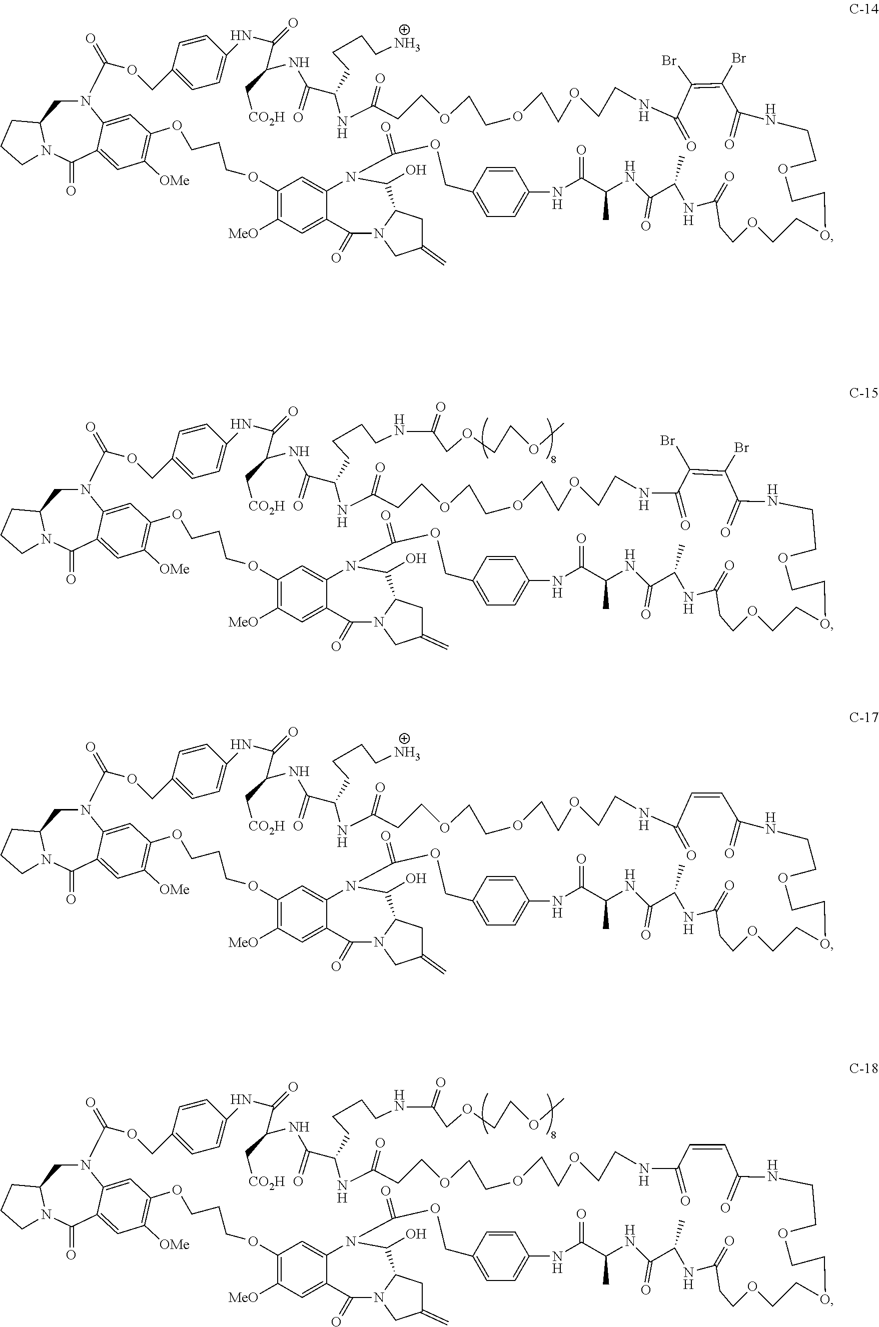 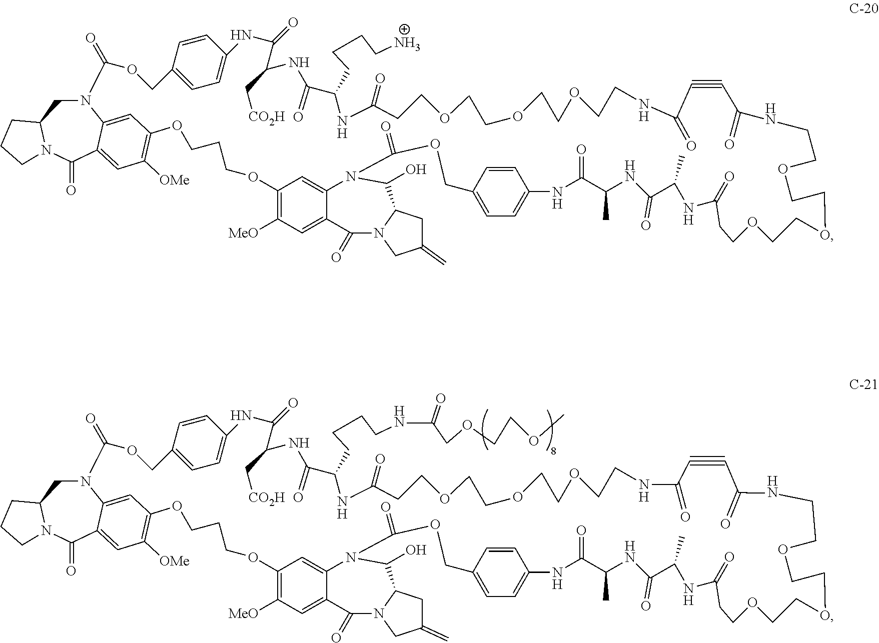 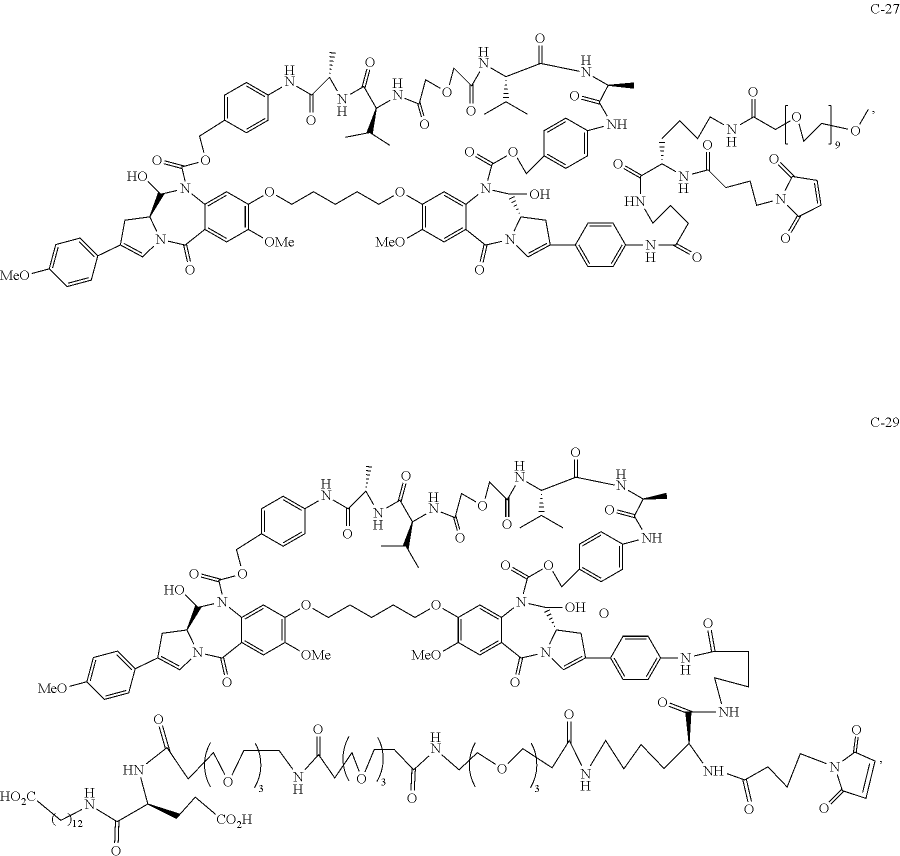
wherein r, r₁, r₂ and r′ are independently 0-200; n is 1-30.

10. The cross-linked PBD dimer derivative or its conjugate to a cell-binding molecule according to claim 1, wherein L₁ and L₂ are independently composed of:
(A): one or more components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)-cyclohexane-1 carboxylate (“MCC”), (4-acetyl)amino-benzoate (“SIAB”), ethyleneoxy (—CH₂CH₂O—) as one or more repeating units (“EO” or “PEO”), 4-thio-butyrate (SPDB), 4-thio-2-hydroxysulfonyl-butyrate (2-Sulfo-SPDB), or natural or unnatural peptides having 1-12 the same or different sequences of natural or unnatural amino acid unites;
(B): one or more of a self-immolative component, a peptidic unit, a hydrazone bond, a disulfide, an ester, an oxime, an amide, or a thioether bond; the self-immolative unit includes aromatic compounds that are electronically similar to the para-aminobenzyl-carbamoyl (PAB) groups, 2-aminoimidazol-5-methanol derivatives, heterocyclic PAB analogs, beta-glucuronides, ortho or para-aminobenzylacetals; or one of the following structures: 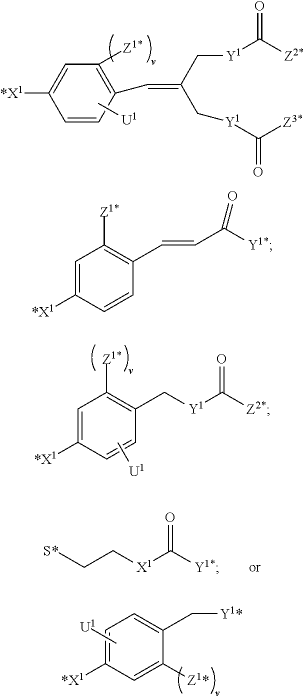
wherein the (*) atom is the point of attachment of another component; X¹, Y¹, Z² and Z³ are independently NH, O, or S; Z¹ is independently H, NHR₁, OR₁, SR₁, COX₁R₁, wherein X₁ and R₁ are defined above; v is O or 1; U¹ is independently H, OH, C₁-C₆ alkyl, (OCH₂CH₂)ₙ, F, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′), wherein R₅ and R₅′ are independently selected from H, C₁-C₈ alkyl; C₂-C₈ alkenyl, alkynyl, heteroalkyl, or amino acid; C₃-C₈ aryl, heterocyclic, carbocyclic, cycloalkyl, heterocycloalkyl, heteroaralkyl, alkylcarbonyl, or glycoside; or pharmaceutical cation salts;
(C): one or more of a non-self-immolative linker component containing one of the following structures: 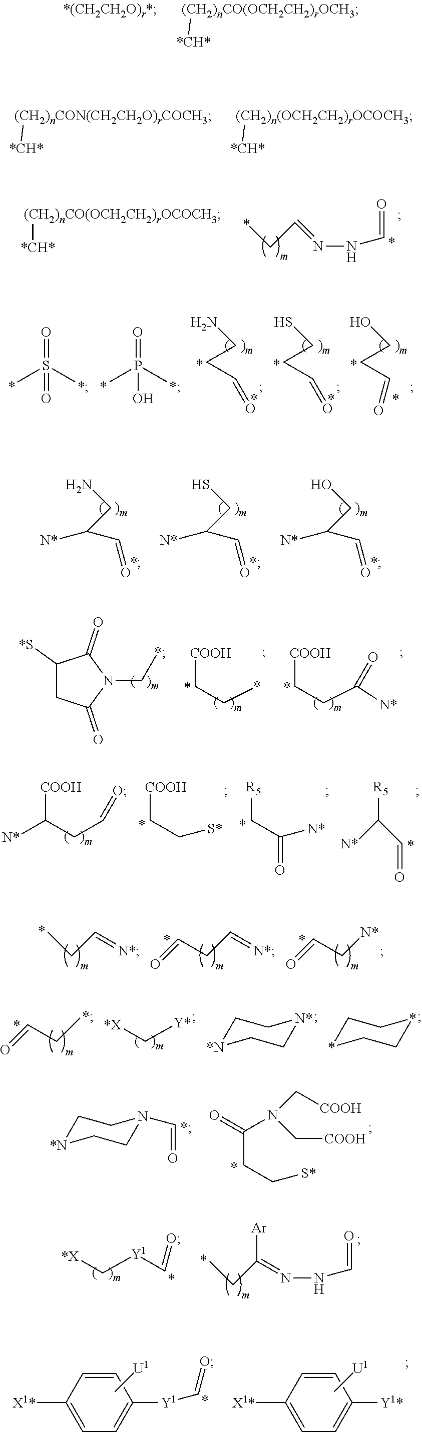 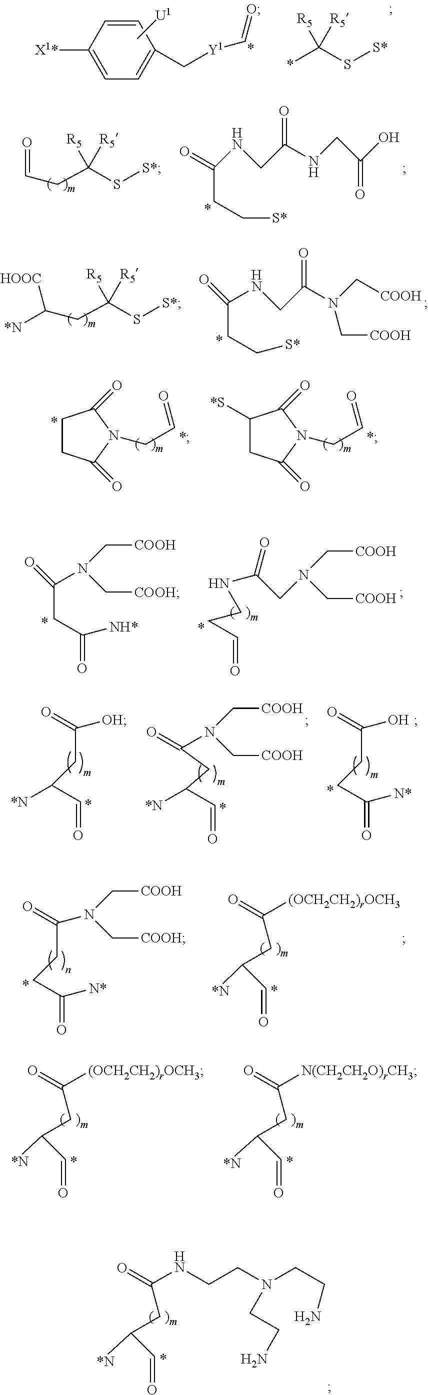 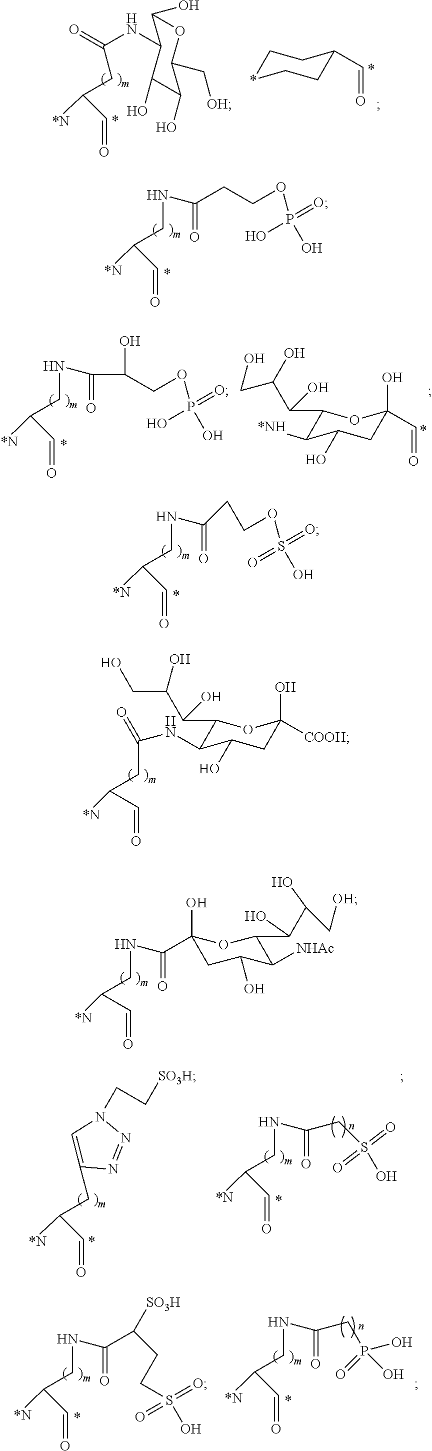
wherein the (*) atom is the site of attachment. X¹, Y¹, U¹, R₅, R₅′ are defined as above; r is 0-100; m and n are 0-20 independently;
(D): one or more of a releasable component that at least one bond can be broken under physiological conditions: a pH-labile, acid-labile, base-labile, oxidatively labile, metabolically labile, biochemically labile or enzyme-labile bond, which having one of the following structures: —(CR₅R₆)ₘ(Aa)ᵣ(CR₇R₈)ₙ(OCH₂CH₂)ₜ—, —(CR₅R₆)ₘ(CR₇R₈)ₙ(Aa)ᵣ(OCH₂CH₂)ₜ—, -(Aa)r-(CR₅R₆)ₘ(CR₇R₈)ₙ(OCH₂CH₂)ₜ—, —(CR₅R₆)ₘ(CR₇R₈)ₙ(OCH₂CH₂)ᵣ(Aa)ₜ-, -(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)ₜ(NR₁₁CO)(CR₉R₁₀)ₙ(OCH₂CH₂)ᵣ—, —(CR₅R₆)ₘ(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂), —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ—(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂), —(CR₅R₆)ₘ(CO)(Aa)ₜ(CR₉R₁₀)ₙ—(OCH₂CH₂)ᵣ—, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)ₜ(CR₇R₈)ᵣ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)ₜ-(CR₇R₈)ₙ—, —(CR₅R₆)—N-methylpiperazin-CO(Aa)ₜ-(CR₇R₈)ₙ—, —(CR₅R)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ₜ, —(CR₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)r (CR₇R₈)ₙ(OCH₂CH₂)ₜ—, —K(CR₅R₆)ₘ—(CR₇R₈)ₙ-(Aa)ᵣ(OCH₂CH₂)ₜ, —K(Aa)ᵣ(CR₅R₆)ₘ(CR₇R₈)ₙ(OCH₂CH₂)ₜ—, —K(CR₅R₆)ₘ(CR₇R₈)ₙ—(OCH₂—CH₂)ᵣ(Aa)ₜ-, —K(CR₅R₆)ₘ(CR₇═CR₈)(CR₉R₁₀)ₙ(Aa)ₜ(OCH₂CH₂)ᵣ—, —K(CR₅R₆)ₘ—(NR₁₁CO)(Aa)ₜ(CR₉R₁₀)ₙ(OCH₂CH₂)ᵣ, —K(CR₅R₆)ₘ(Aa)ₜ(NR₁₁CO)—(CR₉R₁₀)ₙ(OCH₂CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ—(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ-(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ—(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)ₜimidazolyl-CO—(CR₇R₈)ₙ—, —K(CR₅R₆)ₜmorpholinoCO-(Aa)ₜ(CR₇R₈)ₙ—, —K(CR₅R₆)ₜpiperazino-CO(Aa)ₜ(CR₇R₈)ₙ—, —K(CR₅R₆)ₜ—N-methylpiperazin-CO(Aa)ₜ(CR₇R₈)ₙ—, —K(CR₅R)ₘ(Aa)ₜphenyl, —K—(CR₅R₆)ₘ(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘ—N-methylpiperazino(Aa)ₜ-; wherein m, Aa, m, and n are described above; t and r are 0-100 independently; R₃, R₄, R₅, R₆, R₇, and R₈ are independently chosen from H; halide; C₁-C₈ alkyl; C₂-C₈ aryl, alkenyl, alkynyl, ether, ester, amine or amide, which optionally substituted by one or more halide, CN, NR₁R₂, CF₃, OR₁, Aryl, heterocycle, S(O)R₁, SO₂R₁,—CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B, Het (heterocyclic or heteroaromatic ring having C₃-C₈), or peptides containing 1-20 amino acids;
(E): one or more of a hydrophilic structure of following: 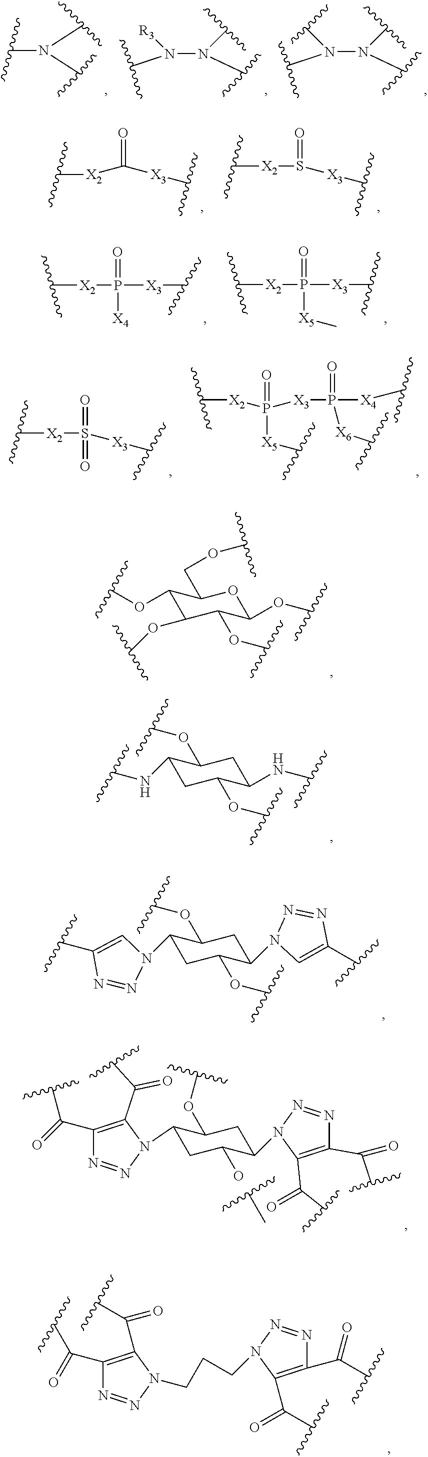 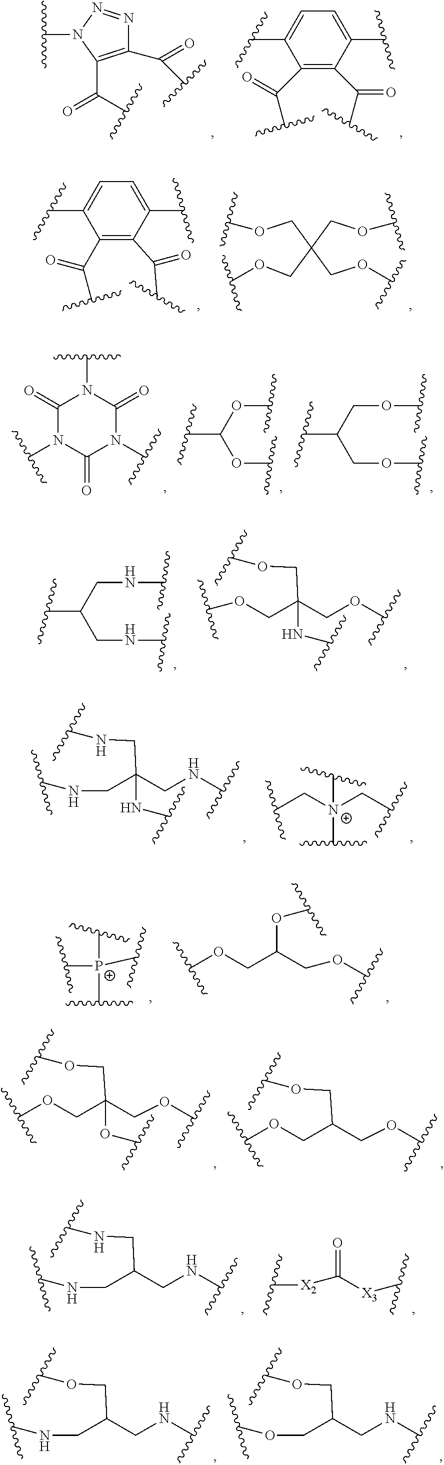
wherein [symbol] is the site of linkage; X₂, X₃, X₄, X₅ or X₆, are independently selected from NH; NHNH; N(R₃); N(R₃)N(R₃′); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or 1-8 of the same or different amino acids; wherein R₃ and R_{3′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ esters, ether, or amide; or polyethyleneoxy unit having formula (OCH₂CH₂)ₚ or (OCH₂CH(CH₃))ₚ, wherein p is an integer from 0 to about 5000, or combination above thereof;
(F): one or more following components linking to E₁ and/or E₂, of Formula (I), (Ia), (Ib), (Ic), (Ie): (III), (II), (III) or (IV); or when Q directly linking to L₁ and/or L₂ of Formula (I), (Ia), (Ib), (Ic), (Ie): (III), (II), (III) or (IV), wherein V₁, and/or V₂, are independently absent: 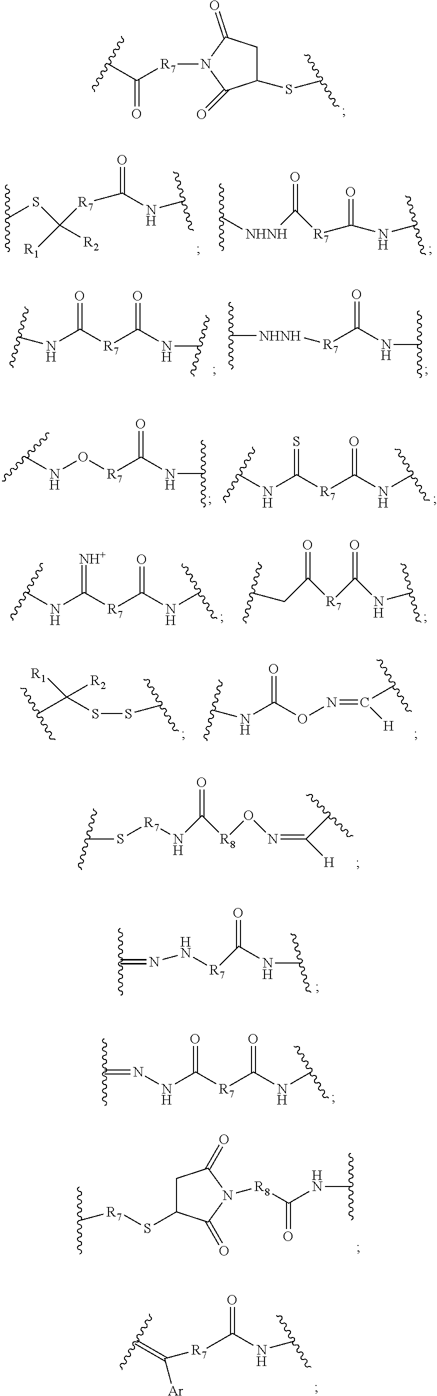 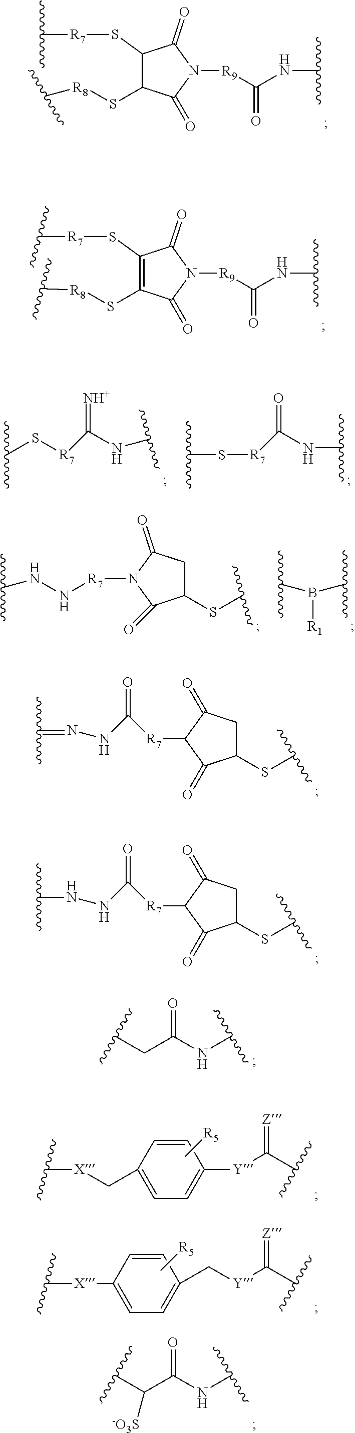 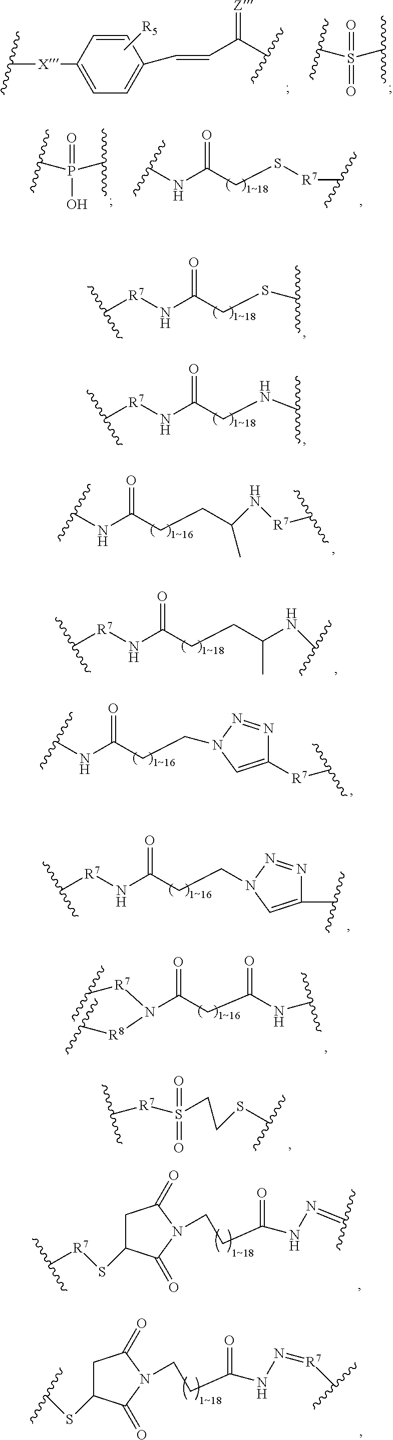 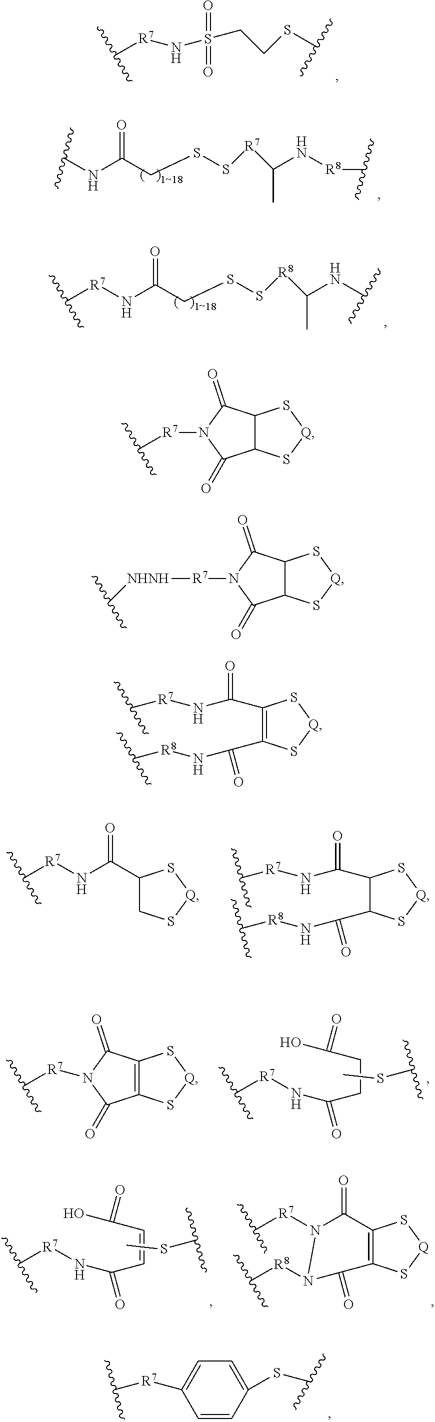 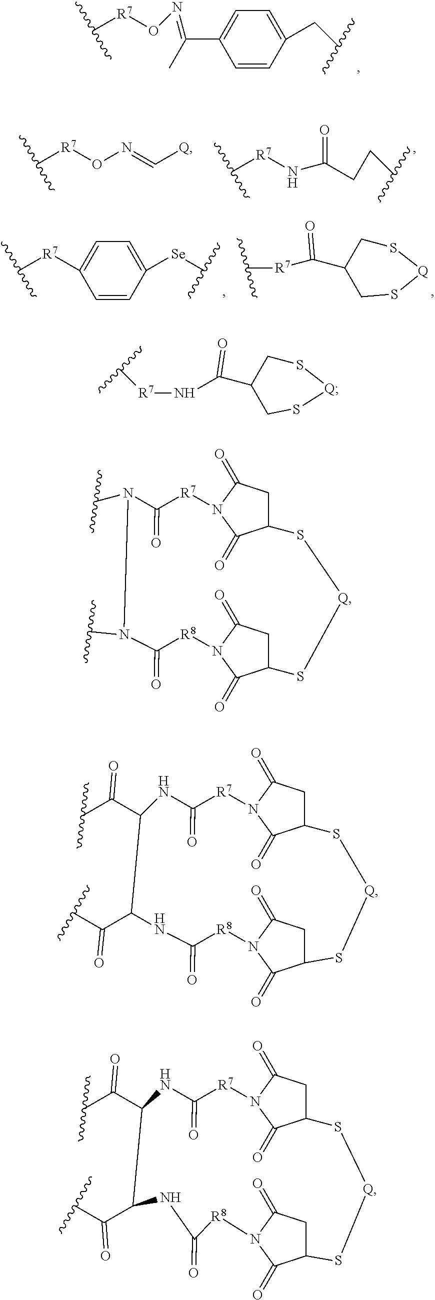 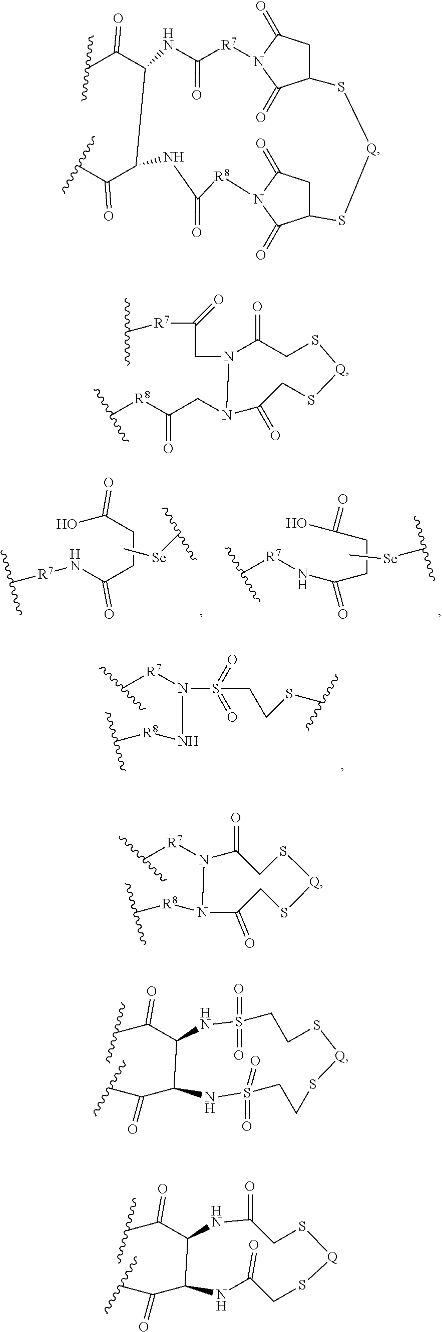
wherein R₇, R₈ and R₉ are independently selected from —C₁-C₈ alkylene-, —C₁-C₇ carbocyclo-, —O—(—C₁-C₈ alkyl)-, -arylene-, —C₁-C₈ alkylene-arylene-, -arylene, —C₁-C₈ alkylene-, —C₁-C₈ alkylene-(C₁-C₈ carbocyclo)-, —(C₃-C₇ carbocyclo)-C₁-C₈ alkylene-, —C₃-C₈ heterocyclo-, —C₁-C₈ alkylene-(C₃-C₈ heterocyclo)-, —(—C₃-C₈ heterocyclo)-C₁-C₉ alkylene-, —(CH₂CH₂O)ₖ—, —(CH(CH₃)CH₂O)ₖ—, and -(CH₂CH₂O)ₖ—CH₂—; k is an integer ranging from 1-30; X′″, Y′″ and Z′″ are independently selected from NH, O or S; Q, R₁ and R₂ are described above.

11. The cross-linked PBD dimer derivative or its conjugate to a cell-binding molecule according to claim 1, wherein the cell binding molecule is selected from:
(A): the group consisting of an antibody, a protein, probody, nanobody, a vitamin (including folate), peptides, a polymeric micelle, a liposome, a lipoprotein-based drug carrier, a nanoparticle drug carrier, a dendrimer, and a molecule or a particle said above coating or linking with a cell-binding ligand, or a combination of said above thereof;
(B): an antibody-like protein, a full-length antibody (polyclonal antibody, monoclonal antibody, antibody dimer, antibody multimer), multispecific antibody (selected from, bispecific antibody, trispecific antibody, or tetraspecific antibody); a single chain antibody, an antibody fragment that binds to the target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune proteins (SIP), a lymphokine, a hormone, a vitamin, a growth factor, a colony stimulating factor, a nutrient-transport molecule, large molecular weight proteins, fusion proteins, kinase inhibitors, gene-targeting agents, nanoparticles or polymers modified with antibodies or large molecular weight proteins;
(C): a cell-binding molecule or receptor agonist selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); -Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); -Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, -acts by targeting follicle stimulating hormone (FSH) and luteinizing hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2NaI-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2NaI-D-4-chloroPhe-D-3-(3-pyridyl)Ala-Ser-(N-Me)Tyr-D-Asn-Leu-isopropylLys-Pro-DAla-NH₂), Cetrorelix (Ac-D-2NaI-D-4-chloroPhe-D-3-(3-pyridyl)Ala-Ser-Tyr-D-Cit-Leu-Arg-Pro-D-Ala-NH₂), Degarelix (Ac-D-2NaI-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2NaI-D-4-chloroPhe-D-3-(3-pyridyl)Ala-Ser-Tyr-D-(N9, N10-diethyl)-homoArg-Leu-(N9, N10-diethyl)-homoArg-Pro-D-Aa-NH₂); Pattern Recognition Receptor (PRRs), selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of α_{V}β₁, α_{V}β₃, α_{V}β₅, α_{V}β₆, α₆β₄, α₇β₁, α_{L}β₂, α_{IIb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; -Nanobody (a derivative of VHH (camelid Ig)); Domain antibodies (dAb, a derivative of VH or VL domain); Bispecific T cell Engager (BiTE, a bispecific diabody); Dual Affinity ReTargeting (DART, a bispecific diabody); Tetravalent tandem antibodies (TandAb, a dimerized bispecific diabody); Anticalin (a derivative of Lipocalins); Adnectins (10th FN3 (Fibronectin)); Designed Ankyrin Repeat Proteins (DARPins); Avimers; EGF receptors, or VEGF receptors' agonists;
(D): a molecule of cell-binding molecule/ligand or a cell receptor agonist selected from the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), -LB15 (GnRH antagonist, Abarelix), LB16 (cobalamin, vitamin B12 analog), LB17 (cobalamin, vitamin B12 analog), LB18 (for αᵥβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor,), LB23 (for an androgen receptor), LB24 (Cilengitide/cyclo(-RGDfV-) for an αᵥ integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) or LB54 (Lapatinib analog) which are shown in the following structures: 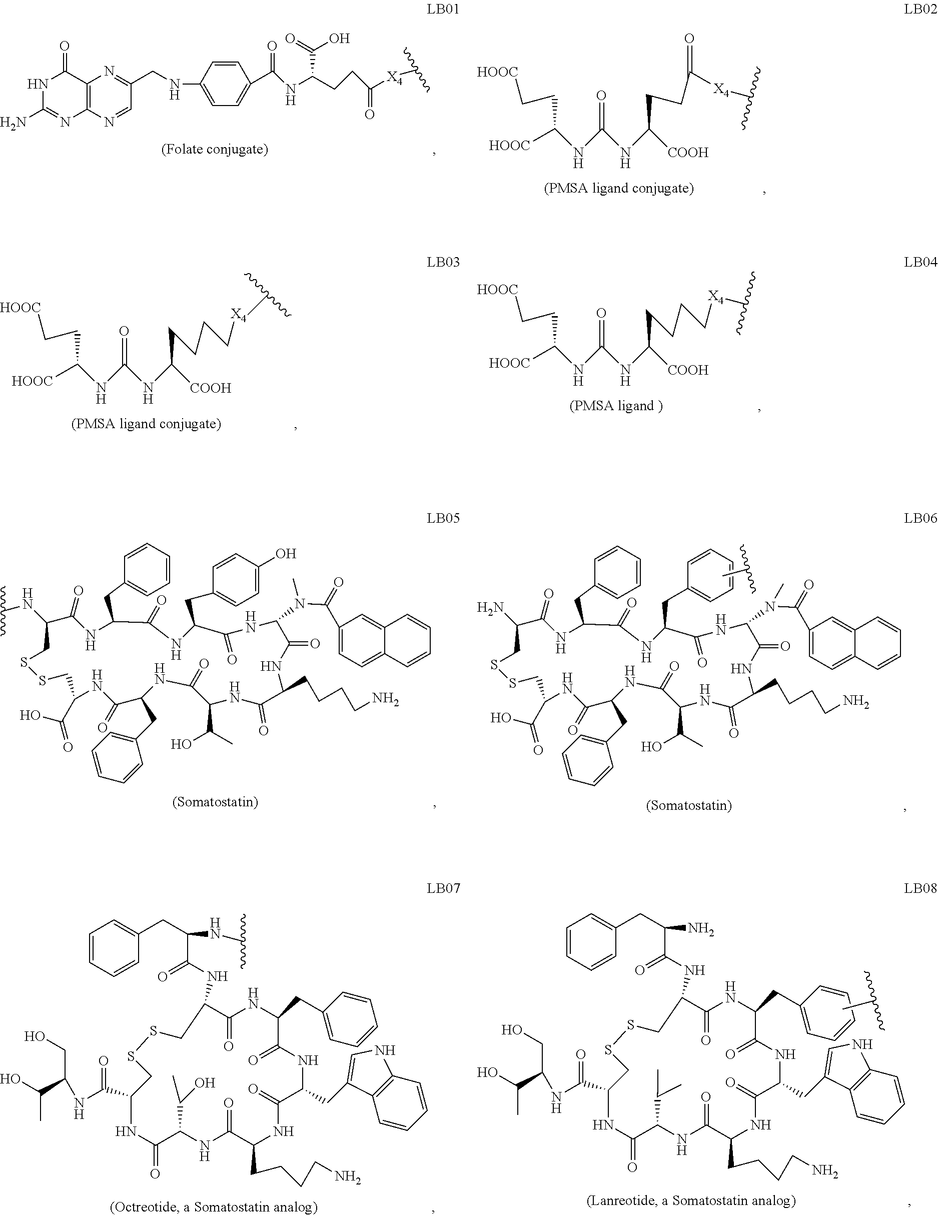 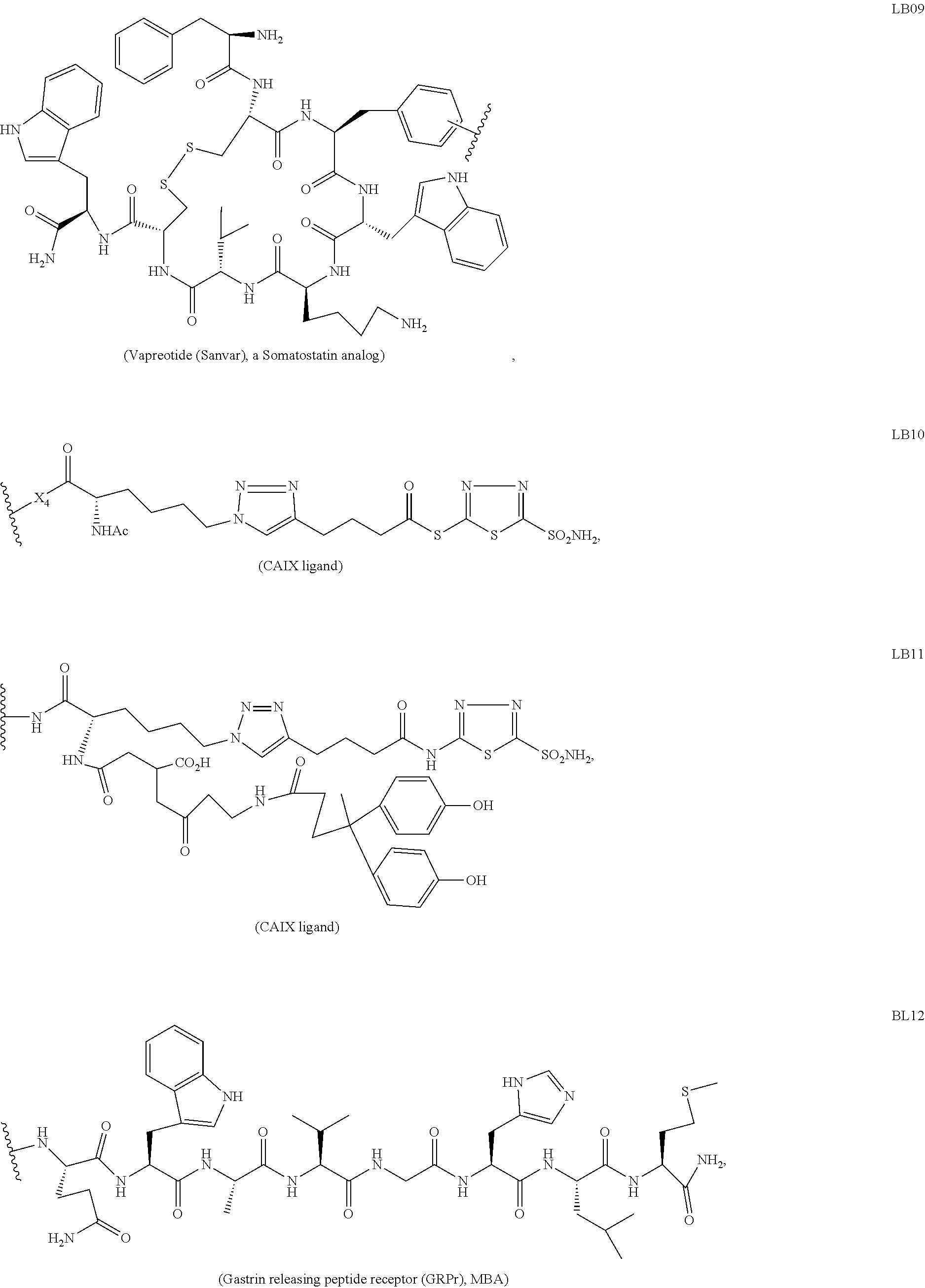 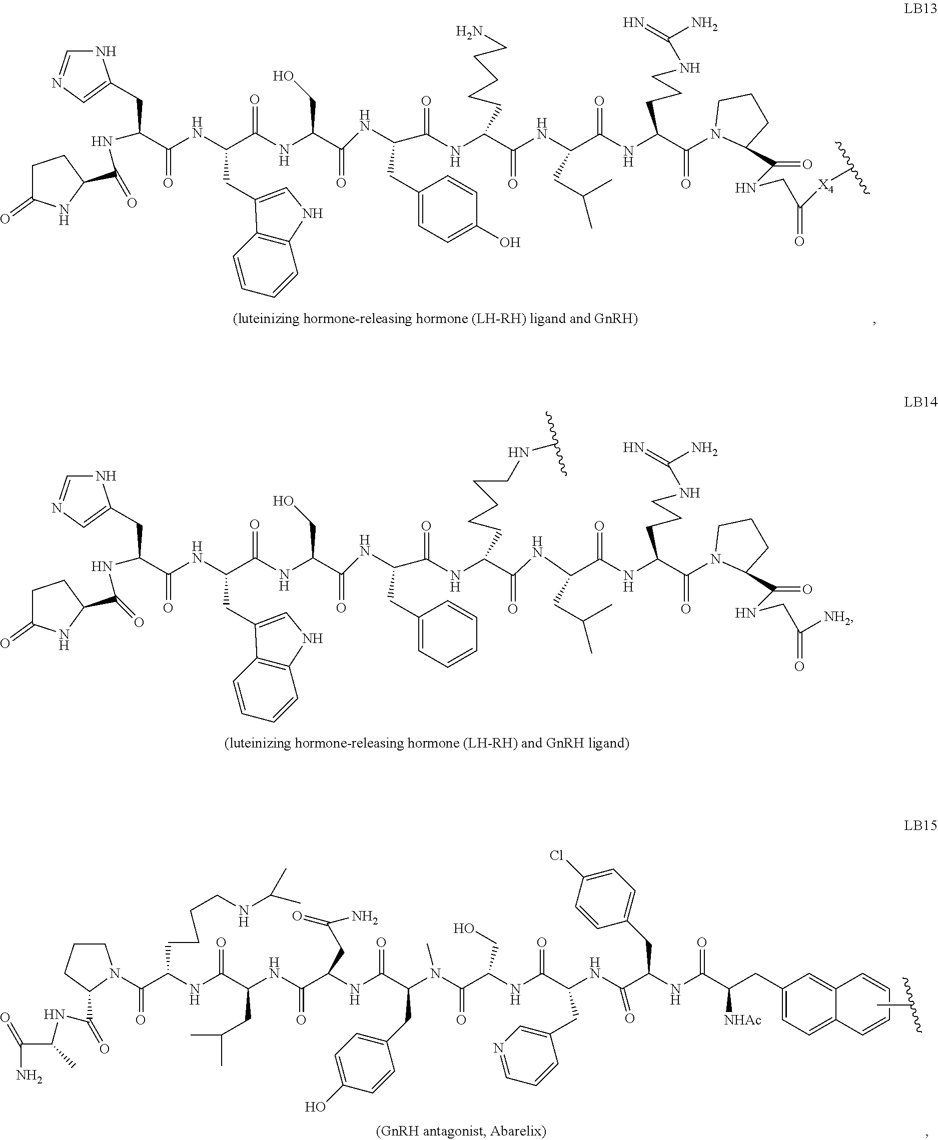 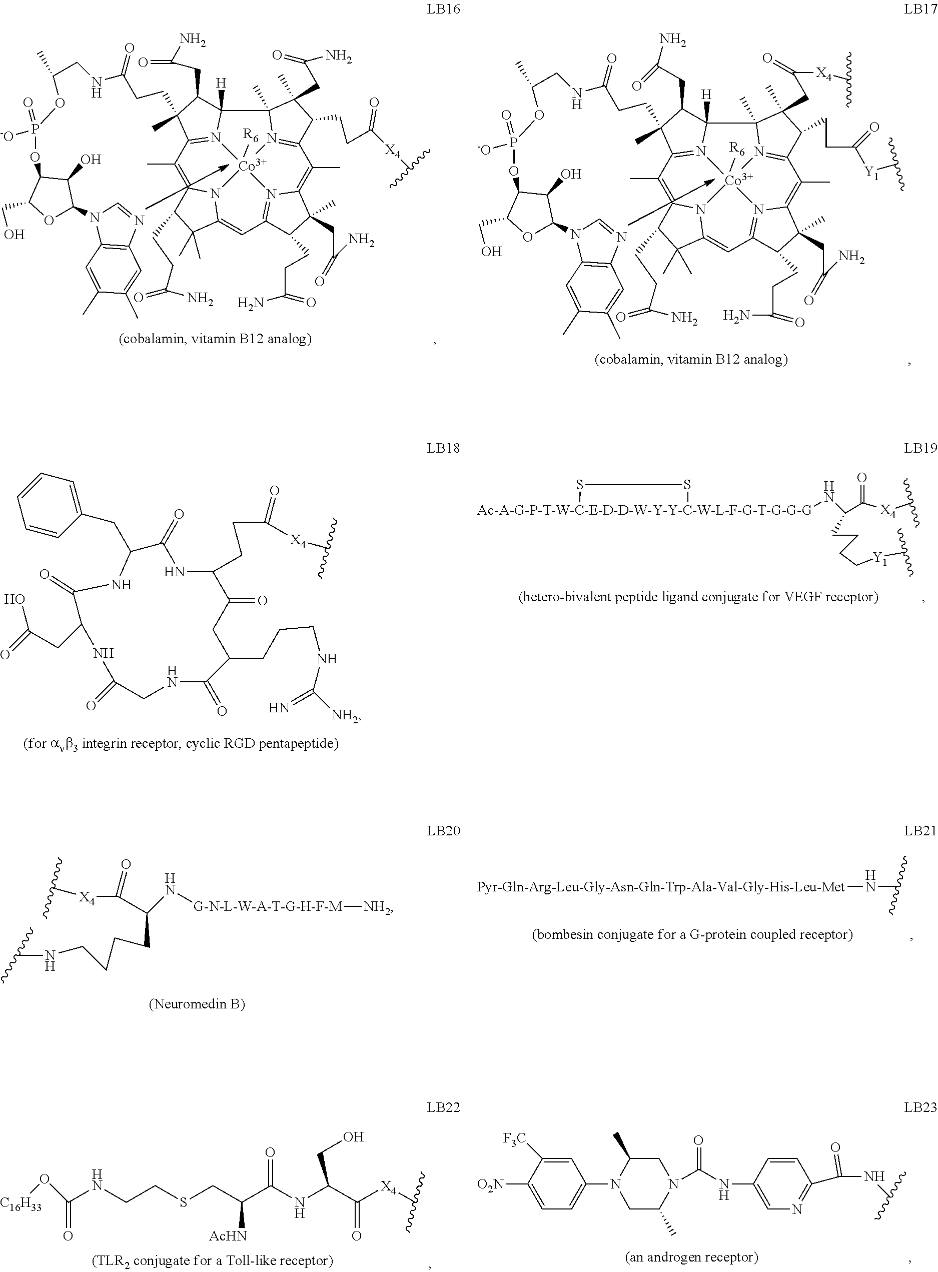 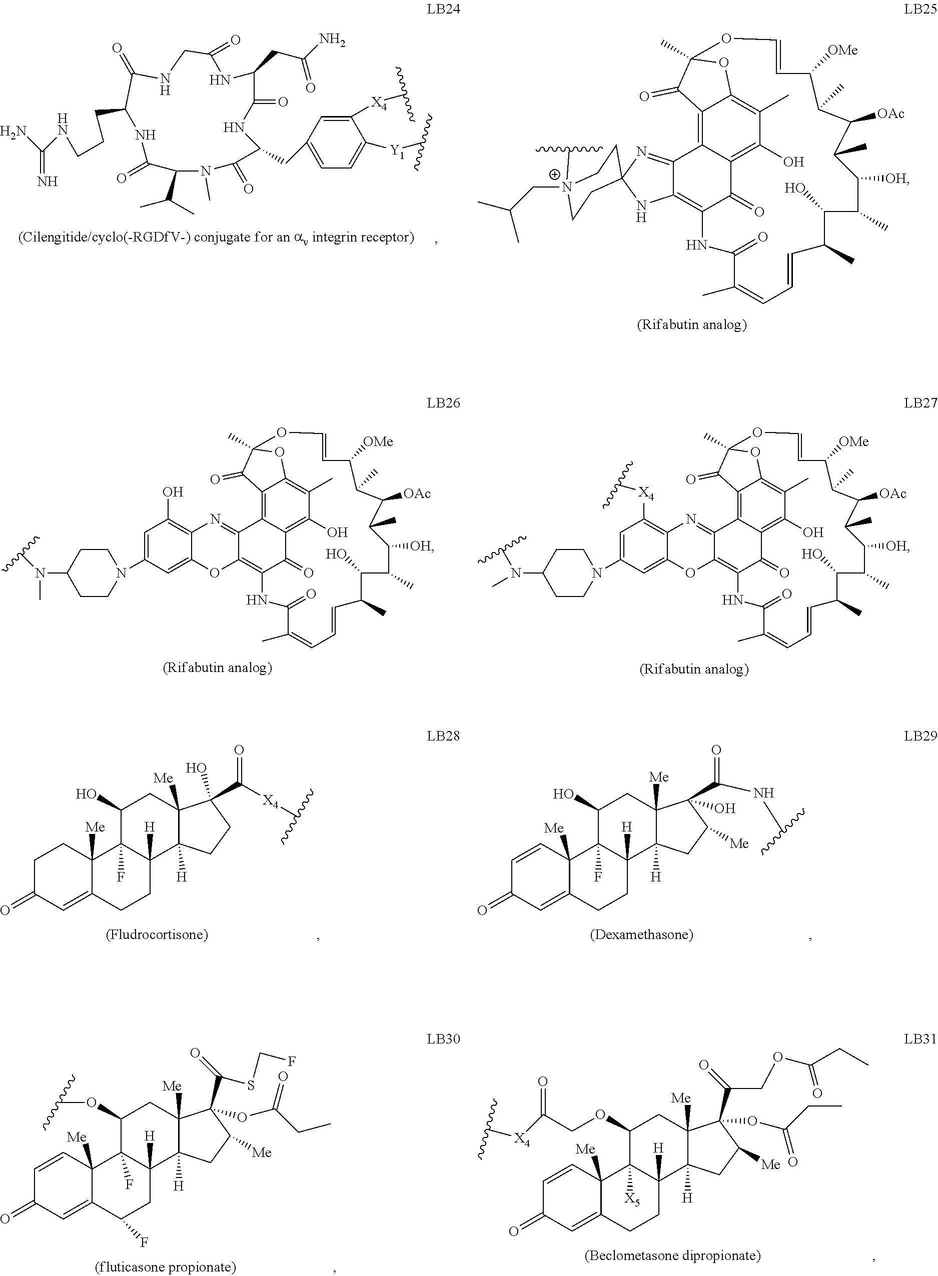 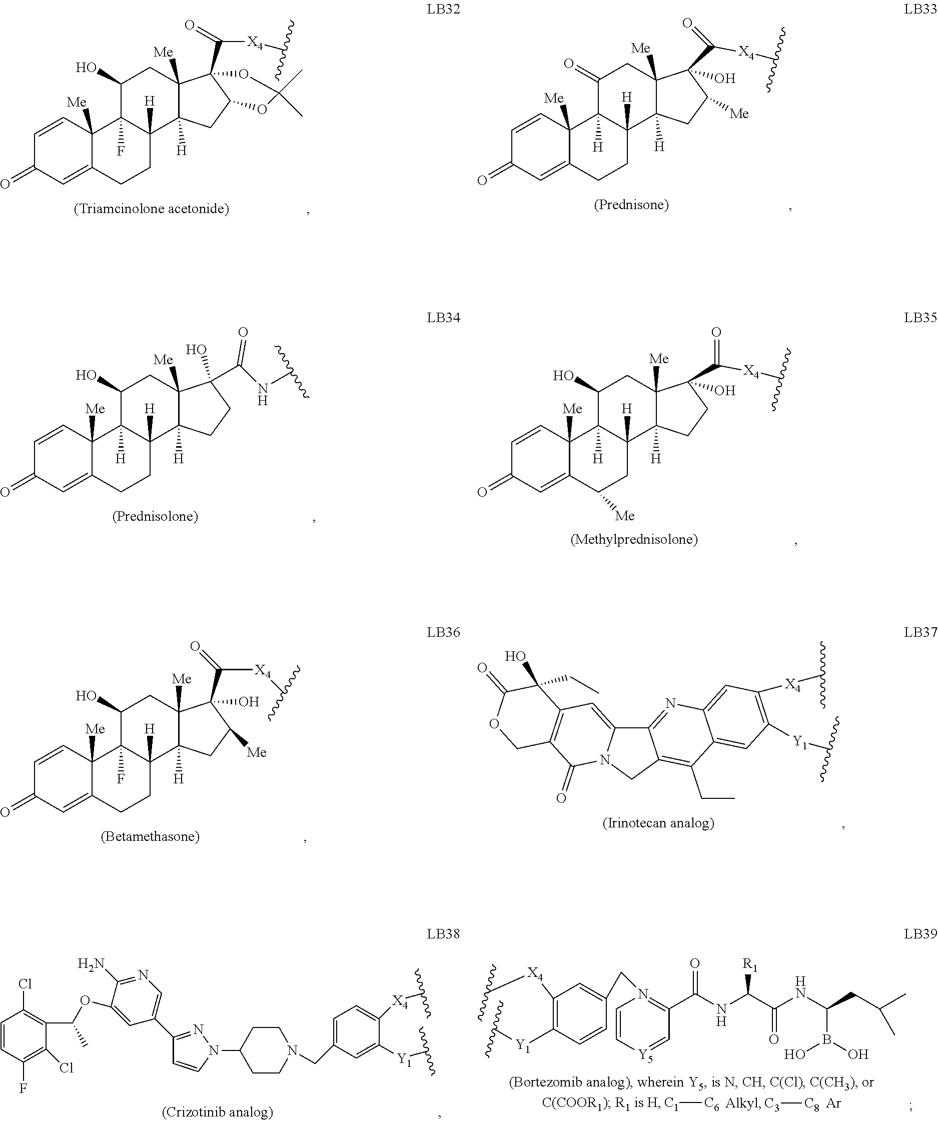 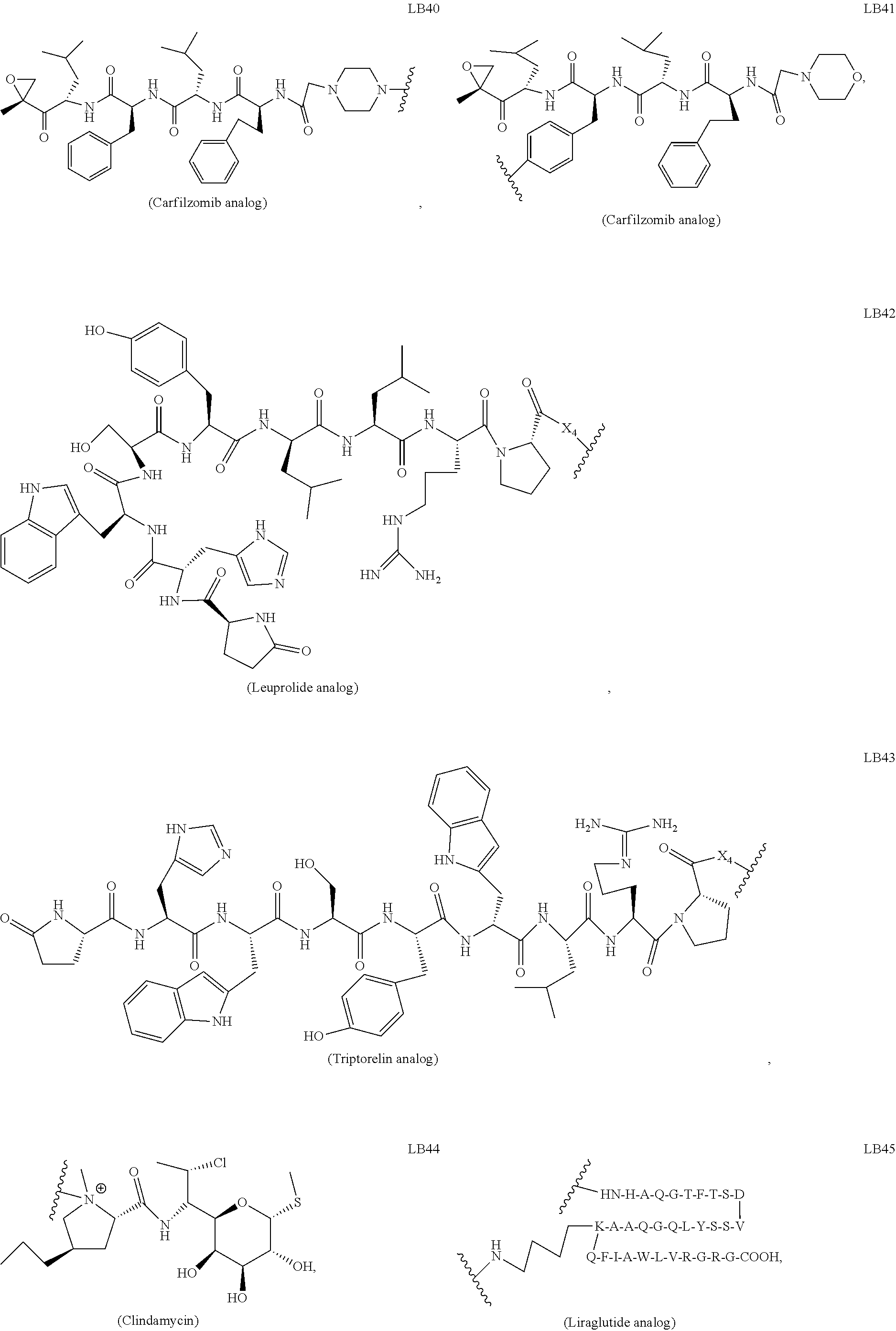 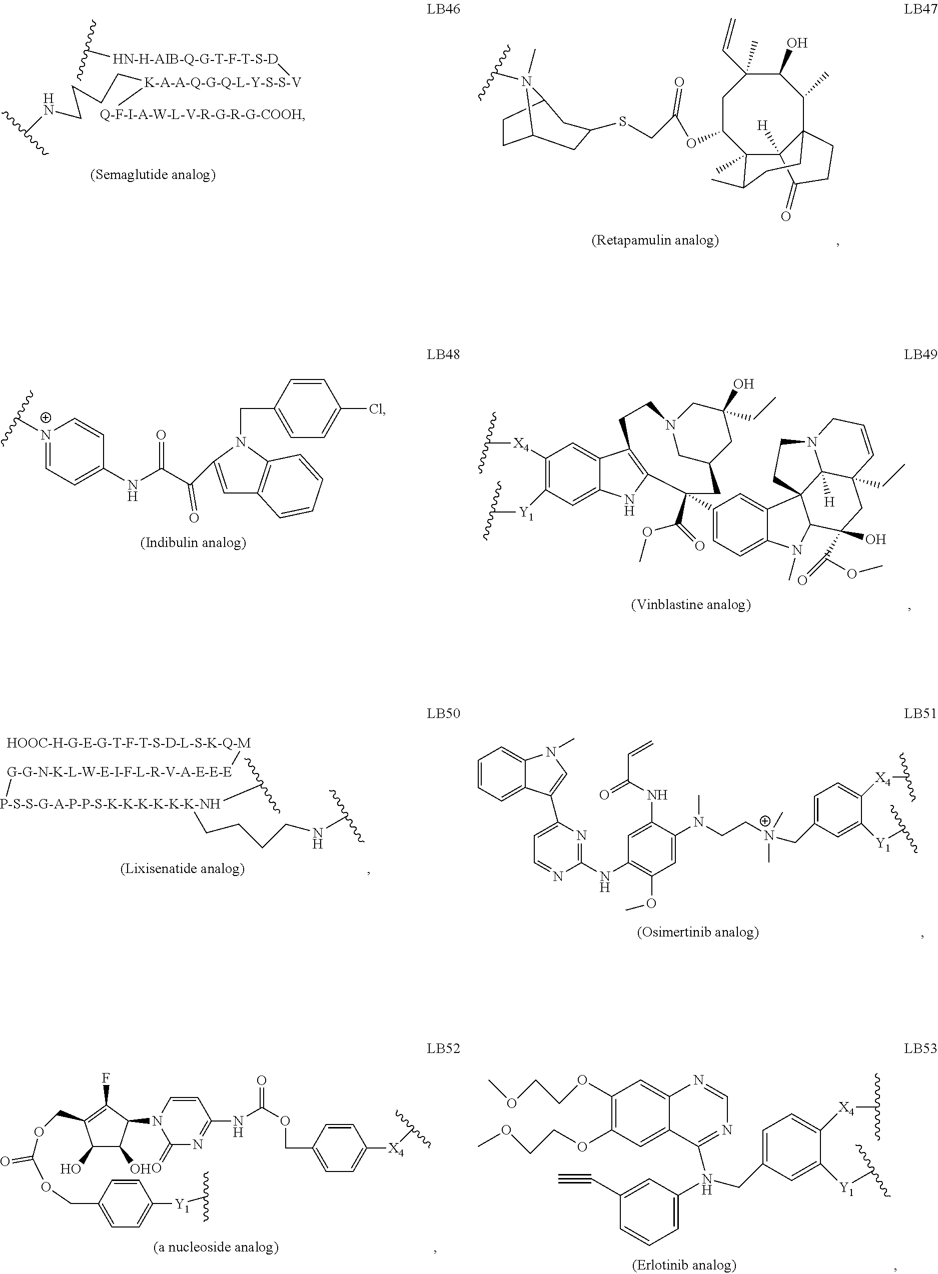 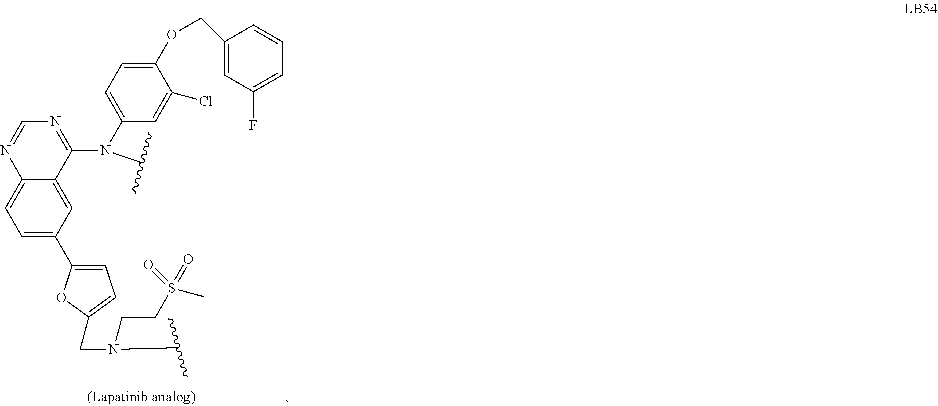
wherein [symbol] is the site to link the PBD dimer derivative through the linker L₁ or/and L₂; X₄, and Y₁ are -independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁; X₁ is H, CH₂, OH, O, C(O), C(O)NH, C(O)N(R₁), R₁, NHR₁, NR₁, C(O)R₁ or C(O)O; X₅ is H, CH₃, F, or Cl; M₁ and M₂ are independently H, Na, K, Ca, Mg, NH₄, N(R₁R₂R₃R₄); R₁, R₂, R₃ and R₄ are defined in claim 1.

12. The cross-linked PBD dimer derivative or its conjugate to a cell-binding molecule according to claim 1, wherein the cell binding molecule is capable of targeting against a tumor cell, a virus infected cell, a microorganism infected cell, a parasite infected cell, an autoimmune disease cell, an activated tumor cells, a myeloid cell, an activated T-cell, an affecting B cell, or a melanocyte, or any cells expressing any one of the following antigens or receptors: CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD12w, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202 (a, b), CD203, CD203c, CD204, CD205, CD206, CD207, CD208, CD209, CD210, CDw210a, CDw210b, CD211, CD212, CD213, CD213a1, CD213a2, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR1, CRGF, CRIPTO, CXCR5, LY64, TDGF1, 4-1BB, -APO2, ASLG659, BMPR1B, 4-1BB, 5AC, 5T4 (Trophoblastic glycoprotein, TPBG, 5T4, Wnt-Activated Inhibitory Factor 1 or WAIF1), Adenocarcinoma antigen, AGS-5, AGS-22M6, Activin receptor-like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C5, C242 antigen, CA125 (carbohydrate antigen 125. MUC16), CA-IX (or CAIX, carbonic anhydrase 9), CALLA, CanAg, Canis lupus familiaris IL31, Carbonic anhydrase IX, Cardiac myosin, CCL11 (C—C motif chemokine 11), CCR₄ (C—C chemokine receptor type 4), CCR₅, CD3E (epsilon), CEA (Carcinoembryonic antigen), CEACAM3, CEACAM5 (carcino-embryonic antigen), CFD (Factor D), Ch4D5, Cholecystokinin 2 (CCK2R), CLDN18 (Claudin-18), Clumping factor A, cMet, CRIPTO, FCSF1R (Colony stimulating factor 1 receptor), CSF2 (colony stimulating factor 2, Granulocyte-macrophage colony-stimulating factor (GM-CSF)), CSP4, CTLA4 (cytotoxic T-lymphocyte-associated protein 4), CTAA16.88 tumor antigen, CXCR4, C-X-C chemokine receptor type 4, cyclic ADP ribose hydrolase, Cyclin E1, CYP11, Cytomegalovirus, Cytomegalovirus glycoprotein B, Dabigatran, DLL3 (delta-like-ligand 3), -DLL4 (delta-like-ligand 4), DPP4 (Dipeptidyl-peptidase 4), DR5 (Death receptor 5), E. coli shiga toxin type-1, E. coli shiga toxin type-2, ED-B, EGFL7 (EGF-like domain-containing protein 7), EGFR, EGFRII, EGFRvIII, Endoglin, Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, -FOLR (folate receptor), Folate receptor alpha, Folate hydrolase, Fos-related antigen 1F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Trans-membrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), -Integrins (α4, α_{IIb}β₃, αvβ3, α₄β₇, α5β1, α6β4, α7β7, αIIβ3, α5β5, αvβ5), Interferon gamma-induced protein, ITGA2, ITGB2, KIR2D, Kappa Ig, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1), PDGF-Rα (Alpha-type platelet-derived growth factor receptor), PDGFR-p, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI)), Rhesus factor, RANKL, RhoC, Ras mutant, RGS5, ROBO4, Respiratory syncytial virus, RON, ROR1 Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF-13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

13. The cross-linked PBD dimer derivative or its conjugate to a cell-binding molecule according to claim 12, wherein the tumor cell is selected from the group consisting of -lymphoma cells, myeloma cells, renal cells, breast cancer cells, prostate cancer cells, ovarian cancer cells, colorectal cancer cells, gastric cancer cells, squamous cancer cells, small-cell lung cancer cells, none small-cell lung cancer cells, testicular cancer cells, malignant cells, or any cells that grow and divide at an unregulated, quickened pace to cause cancers.

14. A pharmaceutical composition comprising a therapeutically effective amount of the cross-linked PBD dimer derivative or its conjugate to a cell-binding molecule of claim 1, which is a conjugate compound, and a pharmaceutically acceptable salt, carrier, diluent, or excipient therefore, or a combination of the conjugates thereof, for treatment or prevention of a cancer, or an autoimmune disease, or an infectious disease.

15. The pharmaceutical composition according to claim 14, which is either in a liquid formula or in a formulated lyophilized solid, comprising by weight of: 0.01%-99% of one or more conjugates claim 14; 0.0%-20.0% of one or more polyols; 0.0%-2.0% of one or more surfactants; −0.0%-5.0% of one or more preservatives; 0.0%-30% of one or more amino acids; 0.0%-5.0% of one or more antioxidants; 0.0%-0.3% of one or more metal chelating agents; 0.0%-30.0% of one or more buffer salts for adjusting pH of the formulation to pH 4.5 to 8.5; and 0.0%-30.0% of one or more of isotonic agent for adjusting osmotic pressure between about 250 to 350 mOsm when reconstituted for administration to a patient;
wherein the polyol is selected from fructose, mannose, maltose, lactose, arabinose, xylose, ribose, rhamnose, galactose, glucose, sucrose, trehalose, sorbose, melezitose, raffinose, mannitol, xylitol, erythritol, maltitol, lactitol, erythritol, threitol, sorbitol, glycerol, or L-gluconate and its metallic salts);
wherein the surfactant is selected from polysorbate 20, polysorbate 40, polysorbate 65, polysorbate 80, polysorbate 81, or polysorbate 85, poloxamer, poly(ethylene oxide)-poly(propylene oxide), polyethylene-polypropylene, Triton; sodium dodecyl sulfate (SDS), sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; or isostearyl ethylimidonium ethosulfate; polyethyl glycol, polypropyl glycol, and copolymers of ethylene and propylene glycol;
wherein the preservative is selected from benzyl alcohol, octadecyldimethylbenzyl ammonium chloride, hexamethonium chloride, benzalkonium chloride, benzethonium chloride, phenol, butyl and benzyl alcohol, alkyl parabens, methyl or propyl paraben, catechol, resorcinol, cyclohexanol, 3-pentanol, or m-cresol;
wherein the amino acid is selected from arginine, cystine, glycine, lysine, histidine, ornithine, isoleucine, leucine, alanine, glycine glutamic acid or aspartic acid;
wherein the antioxidant is selected from ascorbic acid, glutathione, cystine or and methionine;
wherein the chelating agent is selected from EDTA or EGTA;
wherein the buffer salt is selected from sodium, potassium, ammonium, or trihydroxyethylamino salts of citric acid, ascorbic acid, gluconic acid, carbonic acid, tartaric acid, succinic acid, acetic acid or phthalic acid; Tris or tromethamine hydrochloride, phosphate or sulfate; arginine, glycine, glycylglycine, or histidine with anionic acetate, chloride, phosphate, sulfate, or succinate salts;
wherein the tonicity agent is selected from mannitol, sorbitol, sodium acetate, potassium chloride, sodium phosphate, potassium phosphate, trisodium citrate, or sodium chloride.

16. The pharmaceutical composition according to claim 14, which is packed in a vial, bottle, pre-filled syringe, or pre-filled auto-injector syringe, in a form of a liquid or lyophilized powder/solid.

17. The cross-linked PBD dimer derivative or its conjugate to a cell-binding molecule of claim 1, which is a conjugate having in vitro, in vivo or ex vivo cell killing activity.

18. The pharmaceutical composition according to claim 14, further comprising a chemotherapeutic agent, a radiation therapy agent, an immunotherapy agent, an autoimmune disorder agent, an anti-infectious agents or a conjugate for synergistically treatment or prevention of a cancer, or an autoimmune disease, or an infectious disease.

19. The pharmaceutical composition according to claim 18, wherein the chemotherapeutic agent is selected from:
(1). a). an alkylating agent: selected from nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: including busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine];
b). a plant alkaloid: selected from the group consisting of Vinca alkaloids: including vincristine, vinblastine, vindesine, vinorelbine, and navelbin; Taxoids: comprising paclitaxel, docetaxol and their analogs, Maytansinoids including DM1, DM2, DM3, DM4, DM5, DM6, DM7, maytansine, ansamitocins and their analogs, cryptophycins (including the group of cryptophycin 1 and cryptophycin 8); epothilones, eleutherobin, discodermolide, bryostatins, dolostatins, auristatins, tubulysins, cephalostatins; pancratistatin; a sarcodictyin; spongistatin;
c). a DNA Topoisomerase Inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). an antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (including mycophenolic acid, tiazofurin, ribavirin, EICAR); Ribonucleotide reductase Inhibitors: (including hydroxyurea, deferoxamine)]; [Pyrimidine analogs: Uracil analogs: (including ancitabine, azacitidine, 6-azauridine, capecitabine, carmofur, cytarabine, dideoxyuridine, doxifluridine, enocitabine, 5-Fluorouracil, floxuridine, ratitrexed); Cytosine analogs: (including cytarabine, cytosine arabinoside, fludarabine); Purine analogs: (including azathioprine, fludarabine, mercaptopurine, thiamiprine, thioguanine)]; folic acid replenisher, frolinic acid};
e). a hormonal therapy: selected from Receptor antagonists: [Anti-estrogen: (including megestrol, raloxifene, tamoxifen); LHRH agonists: (including goscrclin, leuprolide acetate); Anti-androgens: (including bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (including CB 1093, EB 1089 KH 1060, cholecalciferol, ergocalciferol); Photodynamic therapies: (including verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain)]};
f). a kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib, bafetinib, bosutinib, cabozantinib, vismodegib, iniparib, ruxolitinib, CYT387, axitinib, tivozanib, sorafenib, bevacizumab, cetuximab, Trastuzumab, Ranibizumab, Panitumumab, ispinesib;
g). a poly (ADP-ribose) polymerase (PARP) inhibitors selected from the group of olaparib, niraparib, iniparib, talazoparib, veliparib, CEP 9722 (Cephalon's), E7016 (Eisai's), BGB-290 (BeiGene's), or 3-aminobenzamide.
h). an antibiotic, selected from the group consisting of an enediyne antibiotic (selected from the group of calicheamicin, calicheamicin γ1, δ1, α1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i). a polyketide (acetogenin), bullatacin and bullatacinone; gemcitabine, epoxomicins andcarfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-4-phenylpyridinium ion, Cell cycle inhibitors (including staurosporine), Actinomycins (including Actinomycin D, dactinomycin), amanitins, Bleomycins (including bleomycin A2, bleomycin B2, peplomycin), Anthracyclines (including daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, Histone deacetylase inhibitors ((including Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, selected from the group of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2, 2′,2″-trichlorotriethylamine; trichothecenes (including the group of T-2 toxin, verrucarin A, roridin A and anguidine); urethane, siRNA, antisense drugs;
(2). an anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.
(3). an anti-infectious disease agents comprising:
a). aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). amphenicols: azidamfenicol, chloramphenicol, florfenicol, thiamphenicol;
c). ansamycins: geldanamycin, herbimycin;
d). carbapenems: biapenem, doripenem, ertapenem, imipenem, cilastatin, meropenem, panipenem;
e). cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (including cefoxitin, cefotetan, cefmetazole), oxacephem (flomoxef, latamoxef);
f). glycopeptides: bleomycin, vancomycin (including oritavancin, telavancin), teicoplanin (dalbavancin), ramoplanin;
g). glycylcyclines: tigecycline;
h). β-Lactamase inhibitors: penam (sulbactam, tazobactam), clavam (clavulanic acid);
i). lincosamides: clindamycin, lincomycin;
j). lipopeptides: daptomycin, A54145, calcium-dependent antibiotics (CDA);
k). macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l). monobactams: aztreonam, tigemonam;
m). oxazolidinones: linezolid;
n). penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). polypeptides: bacitracin, colistin, polymyxin B;
p). quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). streptogramins: pristinamycin, quinupristin/dalfopristin;
r). sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). steroid antibacterials: selected from fusidic acid;
t). tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). antibiotics selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, and uvaricin;
(4). anti-viral drugs comprising:
a). entry/fusion inhibitors: aplaviroc, maraviroc, vicriviroc, gp41 (enfuvirtide), PRO 140, CD4 (ibalizumab);
b). integrase inhibitors: raltegravir, elvitegravir, globoidnan A;
c). maturation inhibitors: bevirimat, vivecon;
d). neuraminidase inhibitors: oseltamivir, zanamivir, peramivir;
e). nucleosides & nucleotides: abacavir, aciclovir, adefovir, amdoxovir, apricitabine, brivudine, cidofovir, clevudine, dexelvucitabine, didanosine (ddl), elvucitabine, emtricitabine (FTC), entecavir, famciclovir, fluorouracil (5-FU), 3′-fluoro-substituted 2′, 3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of β-l-thymidine and β-l-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.
(5). pharmaceutically acceptable salts, acids, derivatives, hydrate or hydrated salt; or a crystalline structure; or an optical isomer, racemate, diastereomer or enantiomer of any of the above drugs.

20. The pharmaceutical composition according to claim 19, wherein the synergistic agents are selected from: Abatacept, abemaciclib, Abiraterone acetate, Abraxane, Acetaminophen/hydrocodone, Acalabrutinib, aducanumab, Adalimumab, ADXS31-142, ADXS-HER2, afatinib dimaleate, aldesleukin, alectinib, alemtuzumab, Alitretinoin, ado-trastuzumab emtansine, Amphetamine/dextroamphetamine, anastrozole, Aripiprazole, anthracyclines, Aripiprazole, Atazanavir, Atezolizumab, Atorvastatin, Avelumab, Axicabtagene ciloleucel, axitinib, belinostat, BCG Live, Bevacizumab, bexarotene, blinatumomab, Bortezomib, bosutinib, brentuximab vedotin, brigatinib, Budesonide, Budesonide/formoterol, Buprenorphine, Cabazitaxel, Cabozantinib, capmatinib, Capecitabine, carfilzomib, chimeric antigen receptor-engineered T (CAR-T) cells, Celecoxib, ceritinib, Cetuximab, Chidamide, Ciclosporin, Cinacalcet, crizotinib, Cobimetinib, Cosentyx, crizotinib, CTL019, Dabigatran, dabrafenib, dacarbazine, daclizumab, dacomotinib, daptomycin, Daratumumab, Darbepoetin alfa, Darunavir, dasatinib, denileukin diftitox, Denosumab, Depakote, Dexlansoprazole, Dexmethylphenidate, Dexamethasone, DigniCap Cooling System, Dinutuximab, Doxycycline, Duloxetine, Duvelisib, durvalumab, elotuzumab, Emtricibine/Rilpivirine/Tenofovir, disoproxil fumarate, Emtricitbine/tenofovir/efavirenz, Enoxaparin, ensartinib, Enzalutamide, Epoetin alfa, erlotinib, Esomeprazole, Eszopiclone, Etanercept, Everolimus, exemestane, everolimus, exenatide ER, Ezetimibe, Ezetimibe/simvastatin, Fenofibrate, Filgrastim, fingolimod, Fluticasone propionate, Fluticasone/salmeterol, fulvestrant, gazyva, gefitinib, Glatiramer, Goserelin acetate, Icotinib, Imatinib, Ibritumomab tiuxetan, ibrutinib, idelalisib, ifosfamide, Infliximab, imiquimod, ImmuCyst, Immuno BCG, iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144, lorlatinib, Memantine, Methylphenidate, Metoprolol, Mekinist, mericitabine/Rilpivirine/Tenofovir, Modafinil, Mometasone, Mycidac-C, Necitumumab, neratinib, Nilotinib, niraparib, Nivolumab, ofatumumab, obinutuzumab, olaparib, Olmesartan, Olmesartan/hydrochlorothiazide, Omalizumab, Omega-3 fatty acid ethyl esters, Oncorine, Oseltamivir, Osimertinib, Oxycodone, palbociclib, Palivizumab, panitumumab, panobinostat, pazopanib, pembrolizumab, PD-1 antibody, PD-L1 antibody, Pemetrexed, pertuzumab, Pneumococcal conjugate vaccine, pomalidomide, Pregabalin, ProscaVax, Propranolol, Quetiapine, Rabeprazole, radium 223 chloride, Raloxifene, Raltegravir, ramucirumab, Ranibizumab, regorafenib, ribociclib, Rituximab, Rivaroxaban, romidepsin, Rosuvastatin, ruxolitinib phosphate, Salbutamol, savolitinib, semaglutide, Sevelamer, Sildenafil, siltuximab, Sipuleucel-T, Sitagliptin, Sitagliptin/metformin, Solifenacin, solanezumab, Sonidegib, Sorafenib, Sunitinib, tacrolimus, tacrimus, Tadalafil, tamoxifen, Tafinlar, Talimogene laherparepvec, talazoparib, Telaprevir, talazoparib, Temozolomide, temsirolimus, Tenofovir/emtricitabine, tenofovir disoproxil fumarate, Testosterone gel, Thalidomide, TICE BCG, Tiotropium bromide, Tisagenlecleucel, toremifene, trametinib, Trastuzumab, Trabectedin (ecteinascidin 743), trametinib, tremelimumab, Trifluridine/tipiracil, Tretinoin, Uro-BCG, Ustekinumab, Valsartan, veliparib, vandetanib, vemurafenib, venetoclax, vorinostat, ziv-aflibercept, Zostavax, and their analogs, derivatives, pharmaceutically acceptable salts, carriers, diluents, or excipients thereof, or a combination above thereof.
